(19) United States
(12) Patent Application Publication
(10) Pub. No.: US 2024/0358735 A1
(43) Pub. Date: Oct. 31, 2024

(54) THERAPEUTIC COMPOSITIONS AND RELATED METHODS

(71) Applicants: BOYCE THOMPSON INSTITUTE FOR PLANT RESEARCH, INC., Ithaca, NY (US); CORNELL UNIVERSITY, Ithaca, NY (US)

(72) Inventors: Frank C. SCHROEDER, Ithaca, NY (US); Chester J. J. WROBEL, Ithaca, NY (US); Brian J. CURTIS, Ithaca, NY (US); Jingfang YU, Ithaca, NY (US); Pedro RODRIGUES, Ithaca, NY (US); Arnaud TAUFFENBERGER, Ithaca, NY (US); Bingsen ZHANG, Ithaca, NY (US); Aleksandra SKIRYCZ, Ithaca, NY (US); Venkatesh Periyakavanam THIRUMALAIKUMAR, Ithaca, NY (US)

(21) Appl. No.: 18/681,728

(22) PCT Filed: Aug. 26, 2022
(86) PCT No.: PCT/US22/41757

§ 371 Date: Feb. 6, 2024

Related U.S. Application Data
(60) Provisional application No. 63/237,811, filed on Aug. 27, 2021.

(51) Int. Cl. A61K 31/7076; A61K 31/7028; A61K 31/7068; A61K 31/7072; A61K 31/708; A61K 47/26; C07H 15/22; C07H 17/02

(52) U.S. Cl. CPC A61K 31/7076; A61K 31/7028; A61K 31/7068; A61K 31/7072; A61K 31/708; A61K 47/26; C07H 15/22; C07H 17/02

(57) ABSTRACT

The invention relates to modular glucosides (MOGLs), therapeutic compositions containing such MOGLs and methods of using the same.

### CROSS-REFERENCE TO RELATED APPLICATIONS

The present application claims priority to U.S. Provisional Application No. 63/237,811, filed Aug. 27, 2021, which is incorporated herein by reference in its entirety.

### GOVERNMENT SUPPORT

This invention was made with Government support under Grant Nos. R35GM131877 and U2CES030167 awarded by the National Institutes of Health. The United States Government has certain rights in the invention.

### BACKGROUND OF THE INVENTION

Secondary metabolites derived from plants, fungi and microbes are among the richest sources of therapeutically useful chemical compounds. For example, in the decade between 2000 and 2010, approximately 50% of all NCEs (new chemical entities) approved by the US FDA for use as human drugs were natural products or derivatives of natural products (J Nat Prod. 2012 Mar. 23; 75(3): 311-335).

Recent investigations by the inventors have demonstrated that nematodes are an unexpected and rich source of molecules with diverse biological activities. Meanwhile, as the underlying mechanisms of aging, and a wide range of human health disorders becomes better understood, the need for more selective and efficacious therapeutic and pharmaceutical treatments has never been greater.

The present invention addresses these and other related needs.

### FIELD OF THE INVENTION

This invention pertains to the field of small molecule therapeutics and provides therapeutic compositions and pharmacologically active analogs of compounds first identified in nematodes as well as methods of using the same therapeutically.

### SUMMARY OF THE INVENTION

Among other things, the present invention encompasses the inventors' discovery of a family of novel small molecule metabolites produced by nematodes including C. elegans. The inventors have made important additional observations regarding the production and function of these metabolites including: the biosynthetic processes by which the novel metabolites are produced (and the similarity of those biosynthetic pathways to those known to operate in other more complex animals including humans); the distribution of the new metabolites within the producing organisms' bodies; the levels of excretion (or lack thereof) of the metabolites into the producing organisms' environment; the different absolute and/or relative abundances of the metabolites among different species of producing organism; changes in such abundances at different life stages of the producing organisms; and the changing levels of absolute and/or relative production, accumulation or consumption of these metabolites in response to diverse metabolic and/or environmental stimuli. Based on these insights, the inventors have recognized that administering compositions containing the identified metabolites (or analogs thereof) provides a useful strategy to treat certain diseases and/or improve the health of animals including humans.

The nematode C. elegans has become an important model system for metabolomics and small molecule signaling in animals. These efforts have led to the identification of a large, structurally diverse library of signaling molecules derived from glycosides of the dideoxysugar ascarylose (FIG. 30a).¹⁻⁴ Ascarosides play a central role in the regulation of development and behavior in C. elegans and other nematodes and mediate interactions of nematodes with animals, plants, and microbiota.^{5,6} Examples include the dispersal signal osas #9 (1), in which N-succinylated octopamine is attached to the 4′-position of the ascarylose, the dauer pheromone component ascr #8 (2), incorporating a folate-derived p-aminobenzoic acid moiety, and uglas #11 (3), featuring an N³-glucosylated uric acid moiety (FIG. 30a). Several recent studies demonstrated that carboxylesterase (cest) homologs are responsible for the ester and amide bonds connecting other building blocks to the ascaroside scaffold (FIG. 30a).⁷⁻⁹ CEST enzymes belong to the α/β-hydrolase superfamily of serine hydrolases, which includes more than 200 other members in C. elegans and a similar number in mouse and humans, many of which have no characterized function.^{10,11}

The biosynthesis of most cest-dependent ascarosides further depends on the activity of Cel-GLO-1, a Rab GTPase that is required for the formation of lysosome-related organelles (LROs), cellular compartments similar to mammalian melanosomes.^{7,12} Recent comparative metabolomic studies of Cel-glo-1 mutants and wildtype C. elegans led to the discovery of a previously undescribed class of metabolites, a large library of over one hundred modular glucosides (MOGLs).⁷ The MOGLs are derived from combinatorial attachment of a wide range of metabolic building blocks to several different core scaffolds, e.g. indole glucoside (iglu #1 (4), iglu #2 (5)), anthranilic acid glucoside (angl #1 (6), angl #2 (7)), or tyramine glucoside (tyglu #3 (8), tyglu #1 (9), FIG. 30b).^{7,13,14} These scaffolds are decorated with one or two additional building blocks and usually bear a phosphate at the 3 position of the glucose, although smaller amounts of non-phosphorylated derivatives are also found.^{7,14} The biosynthesis of most MOGLs is abolished in Cel-glo-1 mutants, indicating that, like modular ascarosides, their biosynthesis requires the LROs. In contrast to ascarosides, which are excreted into the growth media, MOGLs are primarily retained in the worm body, suggesting that they serve intra-organismal functions.⁷

In the MOGLs, other building blocks are linked to the core scaffolds via ester bonds, suggesting that MOGL biosynthesis may also be mediated by cest homologs. Comparative metabolomic analysis of Cel-cest-4 mutants recently showed that Cel-CEST-4 is required for 6-O-attachment of anthranilic acid in two MOGLs, iglu #3 (10) and iglu #4 (11) (FIG. 30b); however, it remained unclear how enzymatic pathways could furnish a library of over a hundred MOGLs.⁷

Therefore, in one aspect, the present invention encompasses therapeutic compositions comprising a therapeutically effective amount of one or more such metabolites or derivatives or analogs of such metabolites. In certain embodiments, such therapeutic compositions comprise compounds known as Modular Glucosides or MOGLs—a family of small molecules newly identified in nematodes. MOGLs all contain a glucose moiety decorated with specific substituents present in a variety of substitution patterns. The substitution patterns are described herein with reference to the carbon atom of the glucose ring to which such substituents are attached. For reference, the numbering convention used herein to describe these glucose substitution patterns is shown below.

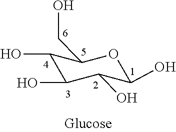 [SMILES: OCC1OC(O)C(O)C(O)C1O]

In most cases, the substituents described herein are attached via covalent bonds to one of the hydroxyl oxygen atoms of the glucose molecule (e.g. through ester, or ether linkages) however, for substituents attached at the 1-position (also referred to as the anomeric position), substituents may either be attached via the oxygen atom, or may be attached via another heteroatom covalently bound to the C1 position—an example of the latter would be an N-linked heterocycle attached to the 1-position.

In certain embodiments, the present invention provides therapeutic compositions comprising one or more MOGLs featuring a glucose molecule having a phosphate group (or a derivative of a phosphate group) at the 3-position of the glucose. In certain embodiments, such MOGLs have additional substitution at one or more of the 1-, 2-, and 6-positions. In certain embodiments, such MOGLs have a phosphate group (or a derivative of a phosphate group) at the 3-position of the glucose and a free —OH group at the 4-position.

In certain embodiments, the present invention provides therapeutic compositions comprising one or more MOGLs featuring a glucose molecule substituted at the 1-, 2-, 3-, and 6-positions, wherein:
the substituent at the 1-position is selected from the group consisting of a nucleobase, an N-linked heterocycle, an acyl- or ether-linked aromatic moiety, an optionally unsaturated acyl group, an ether-linked aromatic moiety substituted with an amine or aminoalkyl group;
the substituent at the 2-position comprises an optionally substituted aromatic, heteroaromatic, or aliphatic acyl group;
the substituent at the 3-position comprises a phosphate, diphosphate, triphosphate, or a derivative of any of these; and
the substituent at the 6-position comprises an acyl-linked amino acid, or an optionally substituted aromatic or aliphatic acyl group;

In certain embodiments, the provided compositions are further characterized in that the glucose derivative is not substituted at the 4-position.

In certain embodiments, such therapeutic compositions comprise MOGLs featuring a glucose derivative substituted at the 1-, 2-, and 3-positions, wherein:
the substituent at the 1-position is selected from the group consisting of a nucleobase, an N-linked heterocycle, an acyl- or ether-linked aromatic moiety, an optionally unsaturated acyl group, an ether-linked aromatic moiety substituted with an amine or aminoalkyl group;
the substituent at the 2-position comprises an optionally substituted aromatic, heteroaromatic, or aliphatic acyl group;
the substituent at the 3-position comprises a phosphate, diphosphate, triphosphate, or a derivative of any of these; and

In certain embodiments, the provided compositions are further characterized in that the glucose derivative is not substituted at the 4- or 6-position.

In certain embodiments, the present invention provides therapeutic compositions comprising MOGLs featuring a glucose derivative substituted at the 1-, 3-, and 6-positions, wherein:
the substituent at the 1-position is selected from the group consisting of a nucleobase, an N-linked heterocycle, an acyl- or ether-linked aromatic moiety, an optionally unsaturated acyl group, an ether-linked aromatic moiety substituted with an amine or aminoalkyl group;
the substituent at the 3-position comprises a phosphate, diphosphate, triphosphate, or a derivative of any of these; and
the substituent at the 6-position comprises an acyl-linked amino acid, or an optionally substituted aromatic or aliphatic acyl group;

In certain embodiments, the provided compositions are further characterized in that the glucose derivative is not substituted at the 2- or 4-position

In certain embodiments, the present invention provides therapeutic compositions comprising MOGLs featuring a glucose derivative substituted at the 1- and 3-positions, wherein:
the substituent at the 1-position is selected from the group consisting of a nucleobase, an N-linked heterocycle, an acyl- or ether-linked aromatic moiety, an optionally unsaturated acyl group, an ether-linked aromatic moiety substituted with an amine or aminoalkyl group; and
the substituent at the 3-position comprises a phosphate, diphosphate, triphosphate, or a derivative of any of these;

In certain embodiments, the provided compositions are further characterized in that the glucose derivative is not substituted at the 2-, 4- or 6-position.

In certain embodiments, the present invention provides therapeutic compositions comprising MOGLs featuring a glucose derivative substituted at the 1-, 2-, and 6-positions, wherein:
the substituent at the 1-position is selected from the group consisting of a nucleobase, an N-linked heterocycle, an acyl- or ether-linked aromatic moiety, an optionally unsaturated acyl group, an ether-linked aromatic moiety substituted with an amine or aminoalkyl group;
the substituent at the 2-position comprises an optionally substituted aromatic, heteroaromatic, or aliphatic acyl group;
the substituent at the 6-position comprises an acyl-linked amino acid, or an optionally substituted aromatic or aliphatic acyl group;

In certain embodiments, the provided compositions are further characterized in that the glucose derivative is not substituted at the 3- or 4-position.

In certain embodiments, the present invention provides therapeutic compositions comprising MOGLs featuring a glucose derivative substituted at the 1- and 2-, positions, wherein:
the substituent at the 1-position is selected from the group consisting of a nucleobase, an N-linked heterocycle, an acyl- or ether-linked aromatic moiety, an optionally unsaturated acyl group, an ether-linked aromatic moiety substituted with an amine or aminoalkyl group;
the substituent at the 2-position comprises an optionally substituted aromatic, heteroaromatic, or aliphatic acyl group;

In certain embodiments, the provided compositions are further characterized in that the glucose derivative is not substituted at the 3-, 4- or 6-position.

In certain embodiments, the present invention provides therapeutic compositions comprising MOGLs featuring a glucose derivative substituted at the 1- and 6-positions, wherein:
the substituent at the 1-position is selected from the group consisting of a nucleobase, an N-linked heterocycle, an acyl- or ether-linked aromatic moiety, an optionally unsaturated acyl group, an ether-linked aromatic moiety substituted with an amine or aminoalkyl group; and
the substituent at the 6-position comprises an acyl-linked amino acid, or an optionally substituted aromatic or aliphatic acyl group;

In certain embodiments, the provided compositions are further characterized in that the glucose derivative is not substituted at the 2-, 3- or 4-position.

In certain embodiments, the present invention provides therapeutic compositions comprising a gluconucleoside. In certain embodiments, the MOGL-containing therapeutic compositions described above are characterized in that the substituent at the 1-position comprises a nucleobase. In certain embodiments, the nucleobase is N-linked to the 1-position of the glucose scaffold. In certain embodiments, the N-linked nucleobase comprises a pyrimidine base. In certain embodiments, the N-linked nucleobase comprises a purine base. In certain embodiments, the N-linked nucleobase comprises a primary nucleobase. In certain embodiments, the N-linked nucleobase is other than a primary nucleobase, or is an analog or adduct of a primary nucleobase. In certain embodiments, the N-linked nucleobase is a methylated nucleobase. In certain embodiments, the N-linked nucleobase is selected from the group consisting of adenine, cytosine, guanine, thymine, and uracil. In certain embodiments, the nucleobase comprises guanine. In certain embodiments, the nucleobase comprises a methylated analog of guanine. In certain embodiments, the nucleobase comprises 6-O-methyl guanine.

In certain embodiments, the MOGL-containing therapeutic compositions described above are characterized in that the substituent at the 1-position comprises an optionally substituted N-linked heterocycle. In certain embodiments, the N-linked heterocycle comprises a 5- or 6-membered ring containing at least one nitrogen atom. In certain embodiments, the N-linked heterocycle contains one or more sites of unsaturation. In certain embodiments, the N-linked heterocycle comprises indole. In certain embodiments, the N-linked heterocycle comprises a substituted indole. In certain embodiments, the N-linked heterocycle comprises a hydroxy indole. In certain embodiments, the N-linked heterocycle comprises serotonin.

In certain embodiments, the MOGL-containing therapeutic compositions described above are characterized in that the substituent at the 1-position comprises an optionally unsaturated acyl group. In certain embodiments, the substituent at the 1-position comprises an alpha beta unsaturated acyl group. In certain embodiments, an acyl substituent at the 1-position comprises a C₃₋₈ aliphatic group with alpha beta unsaturation. In certain embodiments, the substituent at the 1-position comprises crotonate. In certain embodiments, the substituent at the 1-position comprises tiglate. In certain embodiments, the substituent at the 1-position comprises angelate. In certain embodiments, the substituent at the 1-position comprises valerate. In certain embodiments, the substituent at the 1-position comprises acrylate, methacrylate, or cinnamate. In certain embodiments, the substituent at the 1-position comprises 2-imidazoleacrylate. In certain embodiments, the substituent at the 1-position comprises urocanate.

In certain embodiments, the MOGL-containing therapeutic compositions described above are characterized in that the substituent at the 1-position comprises an acyl- or ether-linked aromatic moiety substituted with an amine or an aminoalkyl group. In certain embodiments, the provided compositions are characterized in that the substituent at the 1-position comprises an acyl-linked aromatic moiety substituted with an amine. In certain embodiments, the acyl-linked aromatic moiety comprises a phenyl ring. In certain embodiments, the acyl-linked aromatic moiety comprises a substituted benzoyl group. In certain embodiments, the acyl-linked aromatic moiety comprises an optionally substituted aminobenzoyl group. In certain embodiments, the acyl-linked aromatic moiety comprises anthranilic acid.

In certain embodiments, substituent at the 1-position comprises an ether-linked aromatic moiety substituted with an aminoalkyl group. In certain embodiments, the ether-linked aromatic moiety comprises a phenyl ring. In certain embodiments, substituent at the 1-position comprises a phenolic ether where the phenyl ring of the phenol is substituted with an aminoalkyl group. In certain embodiments, the ether-linked aromatic moiety comprises a phenol substituted with an optionally substituted 2-aminoethyl group. In certain embodiments, the ether-linked aromatic moiety comprises tyramine. In certain embodiments, the ether-linked aromatic moiety comprises octopamine. In certain embodiments, the substituent at the 1-position comprises O-linked serotonin. In certain embodiments, the substituent at the 1-position comprises O-linked N-acetylserotonin (normelatonin). In certain embodiments, the substituent at the 1-position comprises O-linked dopamine. In certain embodiments, the substituent at the 1-position comprises 3-O-linked dopamine. In certain embodiments, the substituent at the 1-position comprises 4-O-linked dopamine. In certain embodiments, the substituent at the 1-position comprises 3-O-linked norepinepherine. In certain embodiments, the substituent at the 1-position comprises 4-O-linked norepinepherine. In certain embodiments, the substituent at the 1-position comprises 3-O-linked epinepherine. In certain embodiments, the substituent at the 1-position comprises 4-O-linked epinepherine.

In certain embodiments, the MOGL-containing therapeutic compositions described above are characterized in that the substituent at the 2-position comprises an optionally substituted aromatic or heteroaromatic acyl group. In certain embodiments, the substituent at the 2-position comprises an optionally substituted benzoate. In certain embodiments, the optionally substituted benzoate is selected from the group consisting of benzoate, anthranilate, and p-hydroxybenzoate. In certain embodiments, the substituent at the 2-position comprises an optionally substituted heteroaromatic acyl group. In certain embodiments, the substituent at the 2-position comprises a heteroaromatic acyl group with a 6-membered heteroaromatic moiety. In certain embodiments, the substituent at the 2-position comprises a pyridine or pyrimidine carboxylate ester. In certain embodiments, the 2-substituent comprises nicotinate. In certain embodiments, the 2-substituent comprises picolinate. In certain embodiments, the 2-substituent comprises isonicotinate. In certain embodiments, the substituent at the 2-position comprises a heteroaromatic acyl group with a 5-membered heteroaromatic moiety. In certain embodiments, the substituent at the 2-position comprises the ester of a pyrrole or imidazole carboxylic acid. In certain embodiments, the substituent at the 2-position is pyrrole-2-carboxylate.

In certain embodiments, the MOGL-containing therapeutic compositions described above are characterized in that the substituent at the 2-position comprises an optionally substituted aliphatic acyl group. In certain embodiments, the acyl group at the 2-position comprises an optionally substituted aliphatic group. In certain embodiments, the acyl group at the 2-position comprises an optionally substituted C₁₋₄₀ aliphatic group, an optionally substituted C₂₋₂₄ aliphatic group, an optionally substituted C₁₂₋₂₄ aliphatic group, an optionally substituted C₂₋₁₈ aliphatic group, an optionally substituted C₂₋₁₂ aliphatic group, an optionally substituted C₂₋₈ aliphatic group, or an optionally substituted C₁₋₆ aliphatic group. In certain embodiments, the optionally substituted acyl group at the 2-position comprises a hydroxylated C₁₋₄₀ aliphatic group. In certain embodiments, the optionally substituted acyl group at the 2-position comprises an epoxidized C₁₋₄₀ aliphatic group. In certain embodiments, such optionally substituted aliphatic groups are saturated. In certain embodiments, such optionally substituted aliphatic groups have one or more sites of unsaturation. In certain embodiments, such unsaturated aliphatic groups have unsaturation adjacent to the carbonyl of the acyl linkage (e.g. they are alpha-beta unsaturated esters). In certain embodiments, an acyl substituent at the 2-position comprises a C₂₋₈ aliphatic group with alpha beta unsaturation. In certain embodiments, the substituent at the 2-position comprises crotonate. In certain embodiments, the substituent at the 2-position comprises tiglate.

In certain embodiments, the substituent at the 2-position comprises angelate. In certain embodiments, the substituent at the 2-position comprises acrylate, methacrylate, 3-methylcrotonate, isocrotonate, or optionally substituted cinnamate.

In certain embodiments, the MOGL-containing therapeutic compositions described above are characterized in that the substituent at the 3-position of the glucose comprises a phosphate—this may be a simple phosphate (e.g. —OPO₃H₂) or may comprise a di-, tri- or higher phosphate (e.g. —O—(P(O₃H)ₙ—H, where n is an integer greater than 1, e.g., 2, 3, 4, 5, 6, 7, 8, 9, or 10), or a phosphate derivative. In certain embodiments, the 3-substituent is phosphate. In certain embodiments, the 3-substituent is diphosphate. In certain embodiments, the 3-substituent is triphosphate. For the synthesis of di- and triphosphate MOGLs, the corresponding mono-phosphates can be synthesized and subsequently converted into diphosphates and triphosphates using, for example, the strategy outlined in Angewandte Chemie-International Edition, 2022, vol. 61, Issue 22 (May 23, 2022, E202201731).

In certain embodiments, the composition is provided in a form wherein the phosphate moiety at the 3-position is protonated. In certain embodiments, the composition is provided in a form wherein the phosphate moiety at the 3-position comprises a salt (e.g. where one or more of —H groups on the phosphate are replaced by a metal cation, organic ‘onium’ or inorganic ‘onium’ group).

In certain embodiments, the MOGL-containing therapeutic compositions described above are characterized in that the substituent at the 3-position of the glucose comprises a sulfate. In certain embodiments, the composition is provided in a form wherein the sulfate moiety at the 3-position comprises a salt (e.g. where one or more of —H groups on the sulfate are replaced by a metal cation, organic ‘onium’ or inorganic ‘onium’ group).

In certain embodiments, the MOGL-containing therapeutic compositions described above are characterized in that the substituent at the 6-position comprises an optionally substituted moiety selected from the group consisting of: an acyl-linked amino acid, an aromatic acyl group and an aliphatic acyl group. In certain embodiments, the substituent at the 6-position comprises an acyl linked amino acid. In certain embodiments, the amino acid is an alpha amino acid. In certain embodiments, the amino acid comprises a proteinogenic amino acid. In certain embodiments, the amino acid comprises one of the 20 encoded proteogenic amino acids. In certain embodiments, the amino acid is phenylalanine. In certain embodiments, the substituent at the 6-position comprises a peptide linked to the glucose via an ester bond.

In certain embodiments, the substituent at the 6-position comprises an aromatic acyl group. In certain embodiments, the substituent at the 6-position comprises an optionally substituted benzoate. In certain embodiments, the optionally substituted benzoate is selected from the group consisting of: benzoate, anthranilate, and p-hydroxybenzoate. In certain embodiments, the substituent at the 6-position comprises anthranilate.

In certain embodiments, the substituent at the 6-position comprises a heteroaromatic acyl group. In certain embodiments, the substituent at the 6-position comprises an optionally substituted heteroaromatic acyl group with a 6-membered heteroaromatic moiety. In certain embodiments, the substituent at the 6-position comprises a pyridine or pyrimidine carboxylate ester. In certain embodiments, the 6-substituent comprises nicotinate. In certain embodiments, the 6-substituent comprises picolinate. In certain embodiments, the 6-substituent comprises isonicotinate. In certain embodiments, the substituent at the 6-position comprises a heteroaromatic acyl group with a 5-membered heteroaromatic moiety. In certain embodiments, the substituent at the 6-position comprises the ester of a pyrrole or imidazole carboxylic acid. In certain embodiments, the substituent at the 6-position is pyrrole-2-carboxylate

In certain embodiments, the MOGL-containing therapeutic compositions described above are characterized in that the substituent at the 6-position comprises an optionally substituted aliphatic acyl group. In certain embodiments, the acyl group at the 6-position comprises an optionally substituted aliphatic group. In certain embodiments, the acyl group at the 6-position comprises an optionally substituted C₁₋₃₀ aliphatic group, an optionally substituted C₂₋₂₄ aliphatic group, an optionally substituted C₁₂₋₂₄ aliphatic group, an optionally substituted C₂₋₁₈ aliphatic group, an optionally substituted C₂₋₁₂ aliphatic group, an optionally substituted C₂₋₈ aliphatic group, or an optionally substituted C₁₋₆ aliphatic group. In certain embodiments, the acyl group at the 6-position comprises phenylacetate. In certain embodiments, such optionally substituted aliphatic groups are saturated. In certain embodiments, acyl groups at the 6-position have one or more sites of unsaturation. In certain embodiments, the 6-substituent comprises an unsaturated aliphatic group having unsaturation adjacent to the carbonyl of the acyl linkage (e.g. they are alpha-beta unsaturated esters). In certain embodiments, an acyl substituent at the 6-position comprises a C₃₋₈ aliphatic group with alpha beta unsaturation. In certain embodiments, the substituent at the 6-position comprises crotonate. In certain embodiments, the substituent at the 6-position comprises crotonate. In certain embodiments, the substituent at the 6-position comprises tiglate. In certain embodiments, the substituent at the 6-position comprises angelate. In certain embodiments, the substituent at the 6-position comprises acrylate, methacrylate, or cinnamate. In certain embodiments, the substituent at the 6-position comprises 2-imidazoleacrylate. In certain embodiments, the substituent at the 6-position comprises urocanate.

In certain embodiments, the present invention encompasses compositions of matter comprising a therapeutically effective amount of one or more compounds of Formula I:

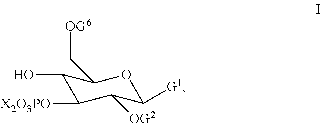

wherein:
G¹ is an optionally substituted moiety selected from the group consisting of: N-linked heterocycle, —OR¹⁰ and —OC(O)R¹¹;
G² is an optionally substituted aliphatic, aromatic, heteroaromatic, or heteroaliphatic acyl group;
X is, independently at each occurrence, selected from the group consisting of —H, an optionally substituted phosphate or polyphosphate moiety, M⁺, and Z⁺;
G⁶ is an optionally substituted aliphatic, aromatic, heteroaromatic, or heteroaliphatic acyl group,
where, R¹⁰ and R¹¹ are each independently selected from the group consisting of: optionally substituted C₁₋₃₂ aliphatic, optionally substituted C₁₋₃₂ heteroaliphatic, optionally substituted aryl, and optionally substituted heteroaryl,
M⁺ is any metal cation, and
Z⁺ is an organic ‘onium’ group comprising at least one nitrogen-, phosphorous-, or sulfur-based cation.

In certain embodiments, a compound of Formula I comprises any one or more of the modular glucosides encompassed by the formula:

For avoidance of the doubt, the depiction above represents the combinatorial range of unique molecules resulting from independently choosing any one of the depicted moieties for attachment to each of the indicated positions by replacement of a dashed line in the figure with a covalent bond.

In certain embodiments, the present invention encompasses compositions of matter comprising a therapeutically effective amount of one or more compounds of Formula I:

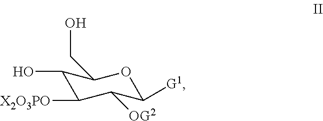

wherein each of G¹, G² and X is as defined above and in the genera and subgenera herein.

In certain embodiments, a compound of Formula II comprises any of the modular glucosides encompassed by the formula:

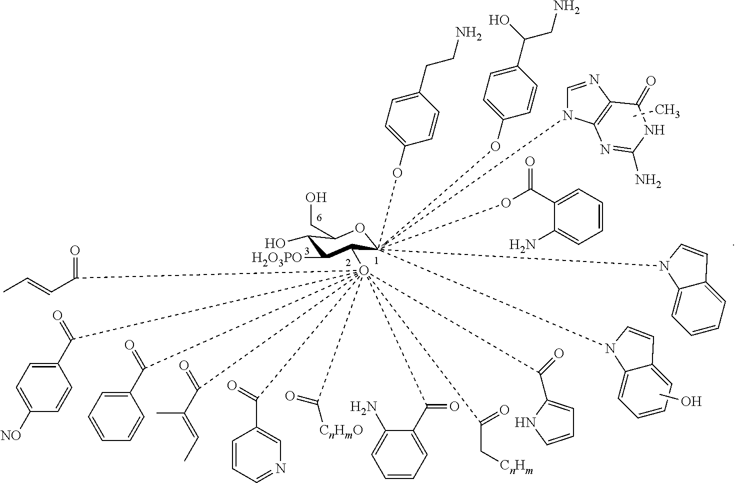

In certain embodiments, the present invention encompasses compositions of matter comprising a therapeutically effective amount of one or more compounds of Formula III:

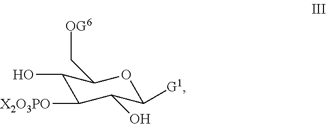

wherein each of G¹, X and G⁶ is as defined above and in the genera and subgenera herein.

In certain embodiments, a compound of Formula III comprises any of the modular glucosides encompassed by the formula:

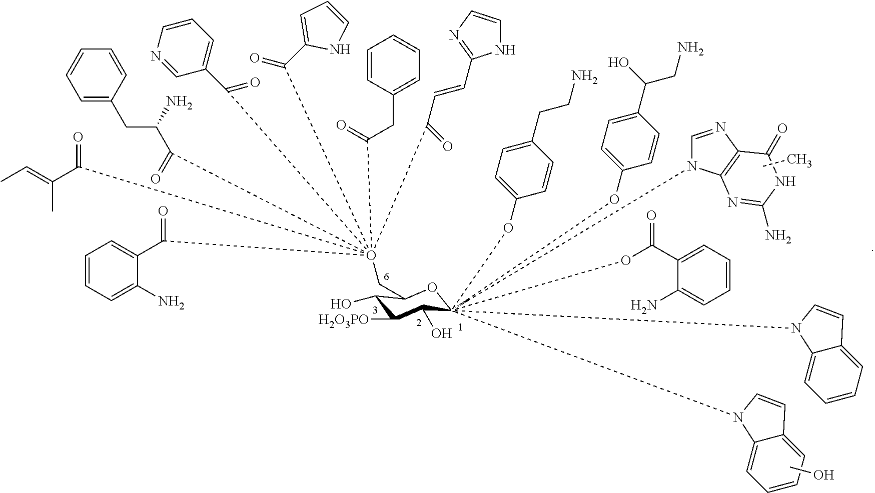

In certain embodiments, the present invention encompasses compositions of matter comprising a therapeutically effective amount of one or more compounds of Formula IV:

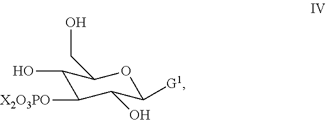

wherein each of G¹ and X is as defined above and in the genera and subgenera herein.

In certain embodiments, a compound of Formula IV comprises any of the modular glucosides encompassed by the formula:

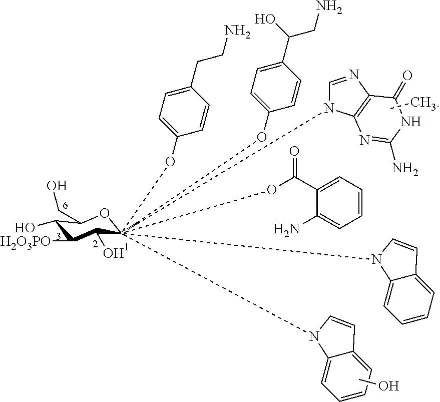

In certain embodiments, the present invention encompasses compositions of matter comprising a therapeutically effective amount of one or more compounds of Formula V:

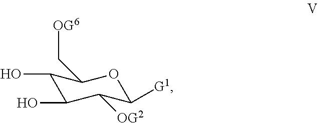

wherein each of G¹, G² and G⁶ is as defined above and in the genera and subgenera herein.

In certain embodiments, a compound of Formula V comprises any of the modular glucosides encompassed by the formula:

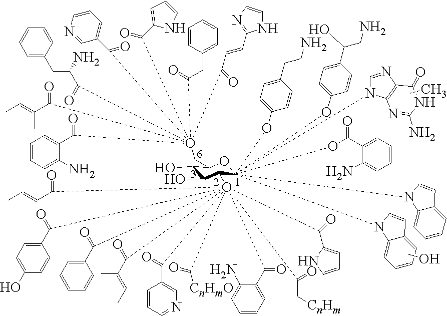

In certain embodiments, the present invention encompasses compositions of matter comprising a therapeutically effective amount of one or more compounds of Formula VI:

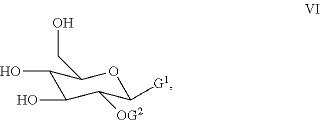

wherein each of G¹ and G² is as defined above and in the genera and subgenera herein.

In certain embodiments, a compound of Formula VI comprises any of the modular glucosides encompassed by the formula:

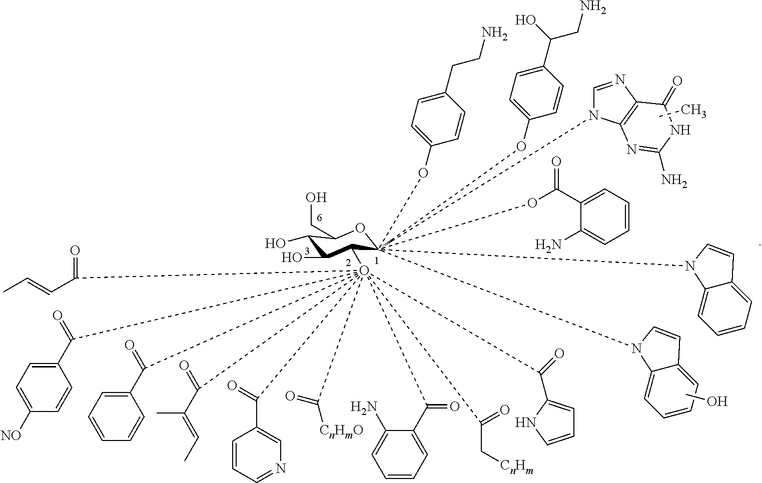

In certain embodiments, the present invention encompasses compositions of matter comprising a therapeutically effective amount of one or more compounds of Formula VII:

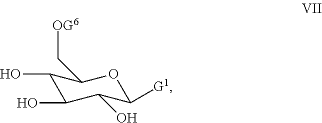

wherein each of G¹, and G⁶ is as defined above and in the genera and subgenera herein.

In certain embodiments, a compound of Formula VII comprises any of the modular glucosides encompassed by the formula:

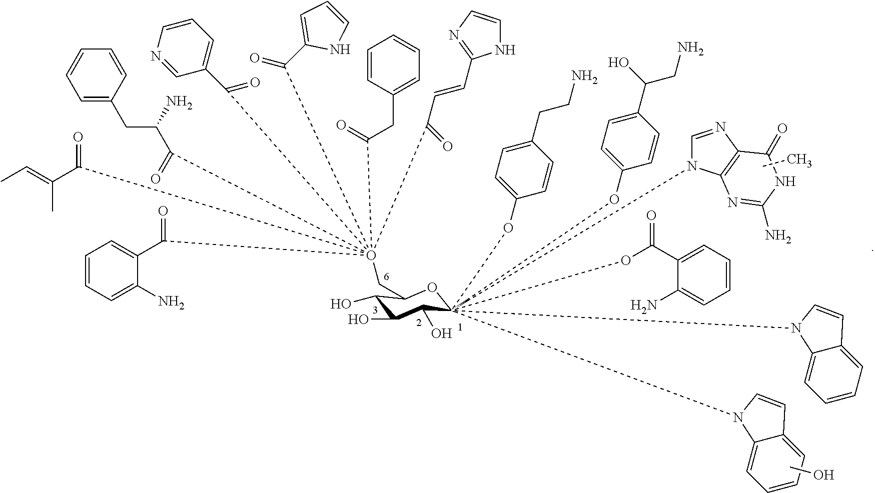

In another aspect, the present invention encompasses methods of improving the health of an animal or of treating or ameliorating a health disorder in an animal by administering to the animal an effective amount of any one or more of the therapeutic compositions described above. In certain embodiments, the method comprises administering such a composition to a mammal. In certain embodiments, the method comprises administering such a composition to a human.

In another aspect, the present invention comprises methods of making therapeutic compositions comprising formulating an effective amount of one or more purified or synthetically-produced MOGLs (or a pharmaceutically-acceptable salt, prodrug or derivative thereof) into a therapeutic composition. In certain embodiments, such therapeutic compositions are selected from the group consisting of: an injectible liquid, a tablet, a capsule, a pill, a solution or suspension for oral administration, a solid dosage form for suspension or dissolution into a drinkable- or injectible liquid, a dermal patch, an eye drop, a cream, an ointment, a gel, a powder, a spray, an inhalable composition, and a nasal spray.

### Definitions

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

Certain compounds of the present invention can comprise one or more asymmetric centers, and thus can exist in various stereoisomeric forms, e.g., enantiomers and/or diastereomers. Thus, inventive compounds and compositions thereof may be in the form of an individual enantiomer, diastereomer or geometric isomer, or may be in the form of a mixture of stereoisomers. Unless otherwise stated, all tautomeric forms of the compounds of the disclosure are within the scope of the invention. In certain embodiments, the compounds of the invention are enantiopure compounds. In certain other embodiments, mixtures of enantiomers or diastereomers are provided.

Furthermore, certain compounds, as described herein may have one or more double bonds that can exist as either a Z or E isomer, unless otherwise indicated. The invention additionally encompasses the compounds as individual isomers substantially free of other isomers and alternatively, as mixtures of various isomers, e.g., racemic mixtures of enantiomers.

In addition to the above-mentioned compounds per se, this invention also encompasses compositions comprising one or more compounds.

Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

As used herein, the term “isomers” includes any and all geometric isomers and stereoisomers. For example, “isomers” include cis- and trans-isomers, E- and Z-isomers, R- and S-enantiomers, diastereomers, (D)-isomers, (L)-isomers, racemic mixtures thereof, and other mixtures thereof, as falling within the scope of the invention. For instance, a compound may, in some embodiments, be provided substantially free of one or more corresponding stereoisomers, and may also be referred to as “stereochemically enriched.”

Where a particular enantiomer is preferred, it may, in some embodiments be provided substantially free of the opposite enantiomer, and may also be referred to as “optically enriched.” “Optically enriched,” as used herein, means that the compound is made up of a significantly greater proportion of one enantiomer. In certain embodiments the compound is made up of at least about 90% by weight of an enantiomer. In some embodiments the compound is made up of at least about 95%, 97%, 98%, 99%, 99.5%, 99.7%, 99.8%, or 99.9% by weight of an enantiomer. In some embodiments the enantiomeric excess of provided compounds is at least about 90%, 95%, 97%, 98%, 99%, 99.5%, 99.7%, 99.8%, or 99.9%. In some embodiments, enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, IN 1972).

The terms “halo” and “halogen” as used herein refer to an atom selected from fluorine (fluoro, —F), chlorine (chloro, —Cl), bromine (bromo, —Br), and iodine (iodo, —I).

The term “acyl” as used herein refers to a group having a formula —C(O)R where R is hydrogen or an optionally substituted aliphatic, carbocyclic, heteroaliphatic, aryl, heteroaryl, or heterocyclic group. In some embodiments, a carbon atom of R is attached to the carbonyl carbon of an acyl group.

The term “aliphatic” or “aliphatic group”, as used herein, denotes a hydrocarbon moiety that may be straight-chain (i.e., unbranched), branched, or cyclic (including fused, bridging, and spiro-fused polycyclic) and may be completely saturated or may contain one or more units of unsaturation, but which is not aromatic. Unless otherwise specified, aliphatic groups contain 1-30 carbon atoms. In certain embodiments, aliphatic groups contain 1-40 carbon atoms. In certain embodiments, aliphatic groups contain 1-24 carbon atoms. In certain embodiments, aliphatic groups contain 1-12 carbon atoms. In certain embodiments, aliphatic groups contain 1-8 carbon atoms. In certain embodiments, aliphatic groups contain 1-6 carbon atoms. In some embodiments, aliphatic groups contain 1-5 carbon atoms, in some embodiments, aliphatic groups contain 1-4 carbon atoms, in yet other embodiments aliphatic groups contain 1-3 carbon atoms, and in yet other embodiments aliphatic groups contain 1-2 carbon atoms. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “unsaturated”, as used herein, means that a moiety has one or more double or triple bonds.

The terms “cycloaliphatic”, used alone or as part of a larger moiety, refer to a saturated or partially unsaturated cyclic aliphatic monocyclic, bicyclic, or polycyclic ring systems, as described herein, having from 3 to 12 members, wherein the aliphatic ring system is optionally substituted as defined above and described herein. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, and cyclooctadienyl. In some embodiments, the cycloaliphatic group has 3-6 carbons. The terms “cycloaliphatic”, also include aliphatic rings that are fused to one or more aromatic or nonaromatic rings, such as decahydronaphthyl or tetrahydronaphthyl, where the radical or point of attachment is on the aliphatic ring. In some embodiments, a cycloaliphatic group is bicyclic. In some embodiments, a cycloaliphatic group is tricyclic. In some embodiments, a cycloaliphatic group is polycyclic.

In some embodiments, a cycloaliphatic group is 3- to 7-membered saturated or partially unsaturated monocyclic carbocyclyl. In some embodiments, a cycloaliphatic group is 4- to 12-membered saturated or partially unsaturated bicyclic carbocyclyl.

The term “alkyl,” as used herein, refers to saturated, straight- or branched-chain hydrocarbon radicals derived from an aliphatic moiety containing between one and six carbon atoms by removal of a single hydrogen atom. Unless otherwise specified, alkyl groups contain 1-12 carbon atoms. In certain embodiments, alkyl groups contain 1-8 carbon atoms. In certain embodiments, alkyl groups contain 1-6 carbon atoms. In some embodiments, alkyl groups contain 1-5 carbon atoms, in some embodiments, alkyl groups contain 1-4 carbon atoms, in yet other embodiments alkyl groups contain 1-3 carbon atoms, and in yet other embodiments alkyl groups contain 1-2 carbon atoms. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like.

The term “alkenyl,” as used herein, denotes a monovalent group derived from a straight- or branched-chain aliphatic moiety having at least one carbon-carbon double bond by the removal of a single hydrogen atom. Unless otherwise specified, alkenyl groups contain 2-12 carbon atoms. In certain embodiments, alkenyl groups contain 2-8 carbon atoms. In certain embodiments, alkenyl groups contain 2-6 carbon atoms. In some embodiments, alkenyl groups contain 2-5 carbon atoms, in some embodiments, alkenyl groups contain 2-4 carbon atoms, in yet other embodiments alkenyl groups contain 2-3 carbon atoms, and in yet other embodiments alkenyl groups contain 2 carbon atoms. Alkenyl groups include, for example, ethenyl, propenyl, butenyl, 1-methyl-2-buten-1-yl, and the like.

The term “alkynyl,” as used herein, refers to a monovalent group derived from a straight- or branched-chain aliphatic moiety having at least one carbon-carbon triple bond by the removal of a single hydrogen atom. Unless otherwise specified, alkynyl groups contain 2-12 carbon atoms. In certain embodiments, alkynyl groups contain 2-8 carbon atoms. In certain embodiments, alkynyl groups contain 2-6 carbon atoms. In some embodiments, alkynyl groups contain 2-5 carbon atoms, in some embodiments, alkynyl groups contain 2-4 carbon atoms, in yet other embodiments alkynyl groups contain 2-3 carbon atoms, and in yet other embodiments alkynyl groups contain 2 carbon atoms. Representative alkynyl groups include, but are not limited to, ethynyl, 2-propynyl (propargyl), 1-propynyl, and the like.

The term “carbocycle” and “carbocyclic ring” as used herein, refers to monocyclic and polycyclic moieties wherein the rings contain only carbon atoms. Unless otherwise specified, carbocycles may be saturated, partially unsaturated or aromatic, and contain 3 to 20 carbon atoms. Representative carbocyles include cyclopropane, cyclobutane, cyclopentane, cyclohexane, bicyclo[2,2,1]heptane, norbomene, phenyl, cyclohexene, naphthalene, spiro[4.5]decane.

The term “aryl” used alone or as part of a larger moiety as in “aralkyl”, “aralkoxy”, or “aryloxyalkyl”, refers to monocyclic and polycyclic ring systems having a total of five to 20 ring members, wherein at least one ring in the system is aromatic and wherein each ring in the system contains three to twelve ring members. The term “aryl” may be used interchangeably with the term “aryl ring”. In certain embodiments of the present invention, “aryl” refers to an aromatic ring system which includes, but is not limited to, phenyl, biphenyl, naphthyl, anthracyl and the like, which may bear one or more substituents. Also included within the scope of the term “aryl”, as it is used herein, is a group in which an aromatic ring is fused to one or more additional rings, such as benzofuranyl, indanyl, phthalimidyl, naphthimidyl, phenantriidinyl, or tetrahydronaphthyl, and the like. The term “aromatic” is not limited to only carbocyclic ring and also encompasses heteroaryl rings as well.

The term “heteroaliphatic,” as used herein, refers to aliphatic groups wherein one or more carbon atoms are independently replaced by one or more atoms selected from the group consisting of oxygen, sulfur, nitrogen, or phosphorus. In certain embodiments, one to six carbon atoms are independently replaced by one or more of oxygen, sulfur, nitrogen, or phosphorus.

Heteroaliphatic groups may be substituted or unsubstituted, branched or unbranched, cyclic or acyclic, and include saturated, unsaturated, or partially unsaturated groups. In some embodiments, a heteroaliphatic group is an aliphatic group having 1-32 (e.g., 1-24, 1-12, 1-8, or 1-6) carbons where 1-6 (e.g., 1-4, 1-3, or 1-2) carbons are independently replaced by a heteroatom selected from oxygen, sulfur, nitrogen, and phosphorus

The terms “heteroaryl” and “heteroar-”, used alone or as part of a larger moiety, e.g., “heteroaralkyl”, or “heteroaralkoxy”, refer to groups having 5 to 14 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, benzofuranyl and pteridinyl. The terms “heteroaryl” and “heteroar-”, as used herein, also include groups in which a heteroaromatic ring is fused to one or more aryl, cycloaliphatic, or saturated or partially unsaturated heterocyclyl rings. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted. In some embodiments, a heteroaryl ring is 5- to 6-membered monocyclic heteroaryl having 1-4 heteroatoms independently selected from nitrogen, oxygen, and sulfur. In some embodiments, a heteroaryl ring is 8- to 10-membered bicyclic heteroaryl having 1-4 heteroatoms independently selected from nitrogen, oxygen, and sulfur.

As used herein, the terms “heterocycle”, “heterocyclyl”, “heterocyclic radical”, and “heterocyclic ring” are used interchangeably and refer to a stable 5- to 7-membered monocyclic or 7-14-membered bicyclic heterocyclic moiety that is either saturated or partially unsaturated, or aromatic (i.e., heteroaryl), and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, pyrrolidonyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a saturated or partically unsaturated heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted. In some embodiments, a heterocylic ring is 3- to 7-membered saturated or partially unsaturated monocyclic heterocyclyl having 1-3 heteroatoms independently selected from nitrogen, oxygen, and sulfur. In some embodiments, a heterocylic ring is 7- to 10-membered saturated or partially unsaturated bicyclic heterocyclyl having 1-4 heteroatoms independently selected from nitrogen, oxygen, and sulfur.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

In some chemical structures herein, substituents are shown attached to a bond which crosses a bond in a ring of the depicted molecule. This means that one or more of the substituents may be attached to the ring at any available position (usually in place of a hydrogen atom of the parent ring structure). In cases where an atom of a ring so substituted has two substitutable positions, two groups may be present on the same ring atom. When more than one substituent is present, each is defined independently of the others, and each may have a different structure. In certain cases where the substituent shown crossing a bond of the ring is —R, this has the same meaning as if the ring were said to be “optionally substituted” as described in the preceding paragraph.

Suitable monovalent substituents on a substitutable carbon atom of an “optionally substituted” group are independently halogen; —(CH₂)₀₋₄R^{∘}; —(CH₂)₀₋₄OR^{∘}; —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)N(R^{∘})₂; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂: —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₈ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or polycyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₄C(O)N(R^{∘})₂; —(CH₂)₀₋₂SR●, —(CH₂)₀₋₂SH, —(CH₂)₀₋₂NH₂, —(CH₂)₀₋₂NHR^{●}, —(CH₂)₀₋₂NR^{●}2, —NO₂, —SiR^{●}₃, —OSiR^{●}₃, —C(O)SR^{●}, —(C₁₋₄ straight or branched alkylene)C(O)OR^{●}, or —SSR● wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃—O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{●} include halogen, —R^{●}, -(haloR^{●}), —OH, —OR^{●}, —O(haloR^{●}), —CN, —C(O)OH, —C(O)OR^{●}, —NH₂, —NHR●, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R, —S(O)₂NR₂, —C(S)NR₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of RT, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of RT are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “isolated” refers to a substance and/or entity that has been (1) separated from at least some of the components with which it was associated when initially produced (whether in nature and/or in an experimental setting) and/or otherwise previously associated, and/or (2) designed, produced, prepared, and/or manufactured by the hand of man. In some embodiments, a substance may be considered to be “isolated” if it is (or has been caused to be) free of or separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of other components (e.g., components with which it was previously associated). In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components (e.g., at least 91%, at least 92%, at least 93%, at least 94%, at least 95%, at least 96%, at least 97%, at least 98%, or at least 99% free of other components). Techniques useful to quantify isolation or purity are known in the art and include standard techniques such as nuclear magnetic resonance or high-performance liquid chromatography. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients.

To give but one example, in some embodiments, a chemical compound that occurs in nature is considered to be “isolated” when, a) by virtue of its origin or source of derivation is not associated with some or all of the components that accompany it in its native state in nature; b) it is substantially free of other chemical compounds, polypeptides, or nucleic acids of the same species from the species that produces it in nature; c) is expressed by or is otherwise in association with components from a cell or other expression system that is not of the species that produces it in nature. Thus, for instance, in some embodiments, a chemical compound that is chemically synthesized or is synthesized in a cellular system different from that which produces it in nature is considered to be an “isolated” compound. Alternatively or additionally, in some embodiments, a compound that has been subjected to one or more purification techniques may be considered to be an “isolated” compound to the extent that it has been separated from other components a) with which it is associated in nature; and/or b) with which it was associated when initially produced.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference.

In certain embodiments, the neutral forms of the compounds are regenerated by contacting the salt with a base or acid and isolating the parent compound in the conventional manner. In some embodiments, the parent form of the compound differs from the various salt forms in certain physical properties, such as solubility in polar solvents.

As used herein, the term “therapeutically effective amount” refers to an amount of a therapeutic agent that confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic agent effective to treat, ameliorate, or prevent a desired disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount is commonly administered in a dosing regimen that may comprise multiple unit doses. For any particular therapeutic agent, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular subject may depend upon a variety of factors including the disorder being treated and the severity of the disorder; the activity of the specific therapeutic agent employed; the specific composition employed; the age, body weight, general health, sex and diet of the subject; the time of administration, route of administration, and/or rate of excretion or metabolism of the specific therapeutic agent employed; the duration of the treatment; and like factors as is well known in the medical arts.

As used herein, the term “treatment” (also “treat” or “treating”) refers to any administration of a substance (e.g., provided compositions) that partially or completely alleviates, ameliorates, relives, inhibits, delays onset of, reduces severity of, and/or reduces incidence of one or more symptoms, features, and/or causes of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

### BRIEF DESCRIPTION OF THE DRAWINGS

FIG. 1 Shows the transmembrane domain prediction for CEST proteins (Cel-CEST-1.2 and Cbr-CEST-2).

FIG. 2 Shows the amino acid sequence alignments of Cel-CEST-1.1 Cel-CEST-1.2 and Cbr-CEST-2. Highlighted sequence shows deletion of Cel-CEST-1.2 mutant

FIG. 3 Shows Partial MS/MS molecular network for MS/MS data (ESI−) of C. elegans endo-metabolome, highlighting features that are strongly downregulated (dark gray) in Cel-cest-1.2 mutants compared to wildtype C. elegans (N₂). Features that did not (cluster single nodes) were omitted.

FIG. 4 Shows Ion chromatograms showing peaks for (a) uglas #11 (3), (b) iglu #4 (11), (c) iglu #3 (10), and (d) iglu #41 (S2) in wildtype (N₂) C. elegans and Cel-cest-1.2 mutants.

FIG. 5 Shows Relative abundances (peak area) of ascarosides relative to wildtype C. elegans (N₂) or C. briggsae (AF16) in Cel-cest-1.2 and Cbr-cest-2 mutants. Bars represent the mean with error bars representing standard deviation.

FIG. 6 Shows (a) MOGL biosynthesis is not significantly reduced in Cel-daf-22 or Cbr-daf-22 mutants compared to wildtype C. elegans (N₂) and wildtype C. briggsae (AF16), respectively. Shown are measured abundances of representative MOGLs in Cel-daf-22 and two different Cbr-daf-22 mutant strains relative to wildtype C. elegans (N₂) and wildtype C. briggsae (AF16). Since these mutant strains do not produce ascr #3,¹⁸ samples were normalized to the abundant iglu #2 (5). (b) MOGL biosynthesis is strongly reduced or abolished in Cel-glo-1 or Cbr-glo-1 mutants. Shown are measured abundances of representative MOGLs in Cel-glo-1 and two different Cbr-glo-1 mutants relative to wildtype C. elegans (N₂) and wildtype C. briggsae (AF16). Most of the shown MOGLs were not detected in any of the Cel-glo-1 or Cbr-glo-1 samples, except for small amounts of iglu #12, angl #401, and angl #36 (arrows) in Cel-glo-1. Bars represent means and error bars standard deviation.

FIG. 7 Shows Ion chromatograms of iglu #141 (S3) and ¹³C₅-labeled iglu #141 in wildtype (N₂) and Cel-daf-22 mutants. Worms were either fed unlabeled L-leucine (black traces) or ¹³C6-L-leucine (two foreground light gray traces).

FIG. 8 Shows (a,b) Ion chromatograms for isomeric mono-acylated MOGLs in wildtype C. elegans (N₂), wildtype C. briggsae (AF16), Cel-cest-1.2 mutants, Cbr-cest-2 mutants, and synthetic samples of the 2-O-acylated isomer, demonstrating selective abolishment of the earlier eluting 2-O-acylated isomer in the mutants. Also shown are MS/MS spectra of the 2-O-acylated isomer from natural and synthetic samples. (a) iglu #3 (10) and iglu #301 (S4); (b) iglu #121 (26) and iglu #12 (S5).

FIG. 9 Shows (c-d) Ion chromatograms for isomeric mono-acylated MOGLs in wildtype C. elegans (N₂), wildtype C. briggsae (AF16), Cel-cest-1.2 mutants, Cbr-cest-2 mutants, and synthetic samples of the 2-O-acylated isomer, demonstrating selective abolishment of the earlier eluting 2-O-acylated isomer in the mutants. Also shown are MS/MS spectra of the 2-O-acylated isomer from natural and synthetic samples. (c) iglu #4 (11) and iglu #401 (28); (d) iglu #10 (S6) and iglu #101 (26).

FIG. 10 Shows BLAST analysis dendrogram relating Cel-CEST-1.1 to homologous predicted proteins in C. briggsae, including Cel-CEST-1.2. Entries in gray represent genes investigated in the current study.

FIG. 11 Shows Ion chromatogram showing levels of C. briggsae-specific MOGL tyglas #9 (S7) in wildtype C. elegans (N₂), wildtype C. briggsae (AF16), Cel-cest-1.2 mutants, and Cbr-cest-2 mutants. The later eluting peak that is increased in Cbr-cest-2 mutants (right side) likely represents the corresponding 6-O-acylated isomer.

FIG. 12 Shows (a-d) Relative peak area of MOGLs at different C. elegans wildtype (N₂) life stages under fed conditions. Different life stages produce different blends of MOGLs. Dots represent the mean and error bars standard deviation.

FIG. 13 Shows (a-d) Relative peak area of MOGLs at different C. elegans wildtype (N₂) life stages under starvation conditions. Different life stages produce different blends of MOGLs, and MOGL blends differ from those produced under fed conditions (FIG. 12). Dots represent the mean and error bars standard deviation.

FIG. 14 Shows (a-d) Peak area in starved relative to fed C. elegans wildtype at different life stages, revealing stark upregulation of the production of many MOGLs under starved conditions. Dots represent the mean and error bars standard deviation.

FIG. 15 Shows C. elegans wildtype (N₂) and Cel-cest-1.2 mutants are phenotypically similar under fed conditions. Time of first egg lay (a),¹⁶ mean lifespan (b), percent death caused by internal hatching (“bagging”) (c), and survival curves (d) of wildtype (N₂) and Cel-cest-1.2 mutant worms under nutritionally replete conditions. Bars represent the mean and whiskers standard error.

FIG. 16 Shows Abundances of MOGLs derived from the indole glucoside or tyramine glucoside scaffolds in wildtype C. elegans fed Providencia alcalifaciens (Jub39) relative to wildtype C. elegans fed E. coli (OP50) diet. Bars represent mean and error bars standard deviation.

FIG. 18 Shows the ¹H NMR spectrum (600 MHz) of iglu #121 (25) in methanol-d₄. ¹H NMR spectrum (600 MHz) of iglu #121 (25) in methanol-d₄.

FIG. 19 Shows the HSQC spectrum (600 MHz) of iglu #121 (25) in methanol-d₄.

FIG. 20 Shows the HMBC spectrum (800 MHz) of iglu #121 (25) in methanol-d₄.

FIG. 21 Shows the dqfCOSY spectrum (600 MHz) of iglu #121 (25) in methanol-d₄.

FIG. 22 Shows the ¹H NMR spectrum (600 MHz) of iglu #401 (28) in methanol-d₄.

FIG. 23 Shows the HSQC spectrum (600 MHz) of iglu #401 (28) in methanol-d₄.

FIG. 24 Shows the HMBC spectrum (600 MHz) of iglu #401 (28) in methanol-d₄.

FIG. 25 Shows the dafCOSY spectrum (600 MHz) of iglu #401 (28) in methanol-d₄.

FIG. 26 Shows the ¹H NMR spectrum (600 MHz) of iglu #101 (26)) in methanol-d₄.

FIG. 27 Shows the HSQC spectrum (600 MHz) of iglu #101 (26)) in methanol-d₄.

FIG. 28 Shows the HMBC spectrum (600 MHz) of iglu #101 (26) in methanol-d₄.

FIG. 29 Shows the dqfCOSY spectrum (600 MHz) of iglu #101 (26) in methanol-d₄.

FIG. 30 Shows the modularity of C. elegans biosynthesis pathways and comparative metabolomics of Cel-cest-1.2 mutants. (a) Assembly of modular ascarosides via CEST enzymes attaching e.g. glucosyl uric acid (Cel-CEST-1.1), p-aminobenzoic acid (Cel-CEST-2.2), indole-3-carboxylic acid (Cel-CEST-3), succinylated octopamine (Cel-CEST-8), and ureidoisobutyric acid (Ppa-UAR-1). (b) Structures of MOGL scaffolds and example MOGLs iglu #3 (10) and iglu #4 (11). (c) Expression levels for Cel-cest-1.2 under fed and starvation conditions.¹⁵ (d) Representative ESI− total ion chromatograms (left) and volcano plot (right) of comparative analysis of the endo-metabolomes of wildtype and Cel-cest-1.2 mutants. (e) Example ESI+ MS/MS spectra, ESI− ion chromatograms, and putative structures of MOGLs from three main scaffold families, tyglu #32 (12), iglu #74 (13), and angl #34 (14). *Cel-cest-1.2-dependent isomer of angl #34 (14). (f) Schematic overview of Cel-cest-1.2 dependent metabolites. Points of attachment of the octopamine, methylguanine or hydroxyindole moieties are not known. New metabolites were named using SMIDs (see Methods and Table S4).

FIG. 31 Shows the characterization of Cel-cest-1.2-dependent metabolites. (a) Abundances of glucoside scaffolds in Cel-cest-1.2 mutants relative to wildtype C. elegans. (b) ESI− ion chromatograms for 2-O-acylated iglu #121 (25) and its 6-O-acylated isomer, iglu #12 (15), in Cel-cest-1.2 and wildtype C. elegans, showing abolishment specifically of the 2-O-acylated isomer. (c) Abundances of 2-O— (baseline gray circles) vs. 6-O— (black bars, white circles) mono-acylated MOGLs in Cel-cest-1.2 and Cbr-cest-2 mutants relative to wildtype C. elegans (N₂) or wildtype C. briggsae (AF16), respectively. Data and error bars represent the mean of 4 biological replicates and standard deviation. (d) Synthetic scheme of 2-O-acylated MOGLs iglu #101 (26), iglu #121 (25), and iglu #401 (28) from iglu #1 (4).

FIG. 32 Shows (a) BLAST analysis dendrogram relating Cel-CEST-1.2 to homologous predicted proteins in other Caenorhabditis species and P. pacificus. Entries Cel-CEST-1.2 and Cbr-CEST-2 were investigated in this study. Percentages represent percent identity with Cel-CEST-1.2. (b) Venn diagram showing representative modular glucosides unique to either C. briggsae (left) or C. elegans (right). (c-d) ESI+ ion chromatograms showing levels of C. briggsae specific, Cbr-CEST-2-dependent MOGLs, tyglu #701 (35, c) and tyglu #131 (37, d) in wildtype C. elegans, wildtype C. briggsae, Cel-cest-1.2 and Cbr-cest-2 mutants.

FIG. 33 Shows Cel-cest-1.2-dependent MOGLs are induced by starvation and Cel-cest-1.2 is required for starvation survival. (a) Quantitation of nicotinic acid- and pyrrolic acid-containing MOGLs in starved relative to fed L3-stage larvae. Inset shows Cel-cest-1.2 expression levels during development. (b) Relative abundances of iglu #42 (39), iglu #58 (40), tyglu #12 (41), and iglu #601 (42) in fed and starved larvae during development. Data points represent the means, and shaded regions standard deviations. (c) Schematic for bioassay, using plates without food. Cel-cest-1.2 mutants exhibit reduced starvation survival due to bagging (a “bursting” of the worm bodies due to internal hatching of larvae). Average time of starvation survival (left) and fraction alive (right) of wildtype C. elegans and Cel-cest-1.2 mutants. (d) Model for MOGL biosynthesis. Scaffolds are glucosylated by putative glucuronosyltransferases (UGTs) and further modified in a combinatorial fashion via CEST homologs that attach diverse building blocks from amino acid and fatty acid metabolism (cross-hashed circle, white circle, right-leaning diagonal line circle) within lysosome related organelles (LROs, lower rounded rectangular box).

FIG. 34 Shows the modularity of MOGL structures and differences in abundance of specific MOGLs in response to starvation for wild type and cest-1.2 mutants of C. elegans.

FIG. 35 Shows measured abundance of proteasome alpha subunits (“PAS”) and proteasome beta-subunits (“PBS”) as measured in proteome samples treated with sngl #1, sngl #2, N-acetylserotonin (NAS), or solvent control (Mock). The TPP data (top bar graph, protein abundance normalized to Mock, heating temperature 53° C.)) demonstrate changed abundance upon treatment with MOGLs sngl #1 and sngl #2. In the LiP-MS experiment (bottom bar graph), specific PAS- or PBS-derived peptides were only detected in the sngl #1- or sngl #2-treated samples, indicative of specific binding to PAS and PBS Error bars, S.D. P values were determined using two-tailed unpaired t-test (P>0.1 are not shown).

FIG. 36 Shows MOGLs sngl #1 and sngl #2, but not the related compound N-acetylserotonin, which lack the glucoside moiety, affect the thermal stability of a proteasome subunit example, PBS-1.

FIG. 37 Shows differential peptides (highlighted in black) in the proteasome subunits of AlphaFold-predicted structures in LiP-MS analyses.

FIG. 38 Shows lifespan curves for C. elegans wildtype and mutants lacking MOGL production via CEST-1.2 or CEST-2.1 on OP50 E. coli.

FIG. 39 Shows mutants lacking MOGL production via CEST-1.2 are sensitive to 300 uM juglone on K12 and tnaA diets. Percent survival of cest-1.2 mutants and wildtype C. elegans (N₂) on 300 uM jugolone fed (a) K12 diet (b) ΔtnaA diet.

### DETAILED DESCRIPTION

### I. Therapeutic Compositions

In one aspect, the present invention encompasses therapeutic compositions comprising a therapeutically effective amount of one or more Modular Glucosides (MOGLs). In certain embodiments, the provided therapeutic compositions comprise one or more MOGLs having a phosphate group (or a derivative of a phosphate group) at the 3-position of the glucose. In certain embodiments, such MOGLs have additional substitutents at one or more of the 1-, 2-, and 6-positions. In certain embodiments, the provided compositions comprise MOGLs having a phosphate group (or a derivative of a phosphate group) at the 3-position of the glucose and a free —OH group at the 4-position.

In certain embodiments, provided therapeutic compositions comprise an effective amount one or more MOGLs featuring a glucose molecule substituted at each of the 1-, 2-, 3-, and 6-positions, wherein:
the substituent at the 1-position is selected from the group consisting of a nucleobase, an N-linked heterocycle, an acyl- or ether-linked aromatic moiety an ether-linked aromatic moiety substituted with an amine or aminoalkyl group;
the substituent at the 2-position comprises an optionally substituted aromatic, heteroaromatic, or aliphatic acyl group;
the substituent at the 3-position comprises a phosphate, diphosphate, triphosphate, or a derivative of any of these; and the substituent at the 6-position comprises an acyl-linked amino acid, or an optionally substituted aromatic or aliphatic acyl group.

In certain embodiments, the provided compositions are further characterized in that the glucose derivative is not substituted at the 4-position.

In certain embodiments, such compositions comprise one or more molecules of Formula I:

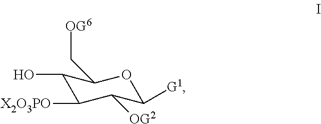

wherein:
G¹ is an optionally substituted moiety selected from the group consisting of: N-linked heterocycle, —OR¹⁰ and —OC(O)R¹¹;
G² is an optionally substituted aliphatic, aromatic, heteroaromatic, or heteroaliphatic acyl group;
X is, independently at each occurrence, selected from the group consisting of —H, an optionally substituted phosphate or polyphosphate moiety, M⁺, and Z⁺;
G⁶ is an optionally substituted aliphatic, aromatic, heteroaromatic, or heteroaliphatic acyl group; and
wherein, R¹⁰ and R¹¹ are each independently selected from the group consisting of: optionally substituted C₁₋₃₂ aliphatic, optionally substituted C₁₋₃₂ heteroaliphatic, optionally substituted aryl, and optionally substituted heteroaryl.

It will be appreciated that throughout the disclosure, references such as “aliphatic, aromatic, heteroaromatic, or aliphatic acyl group” and the like at the 2- and 6-positions of the glucose ring (e.g., G² and G⁶) has the meaning “aliphatic acyl, aromatic acyl, heteroaromatic acyl, or aliphatic acyl group.”

In certain embodiments, a compound of Formula I comprises any one or more of the modular glucosides encompassed by the formula:

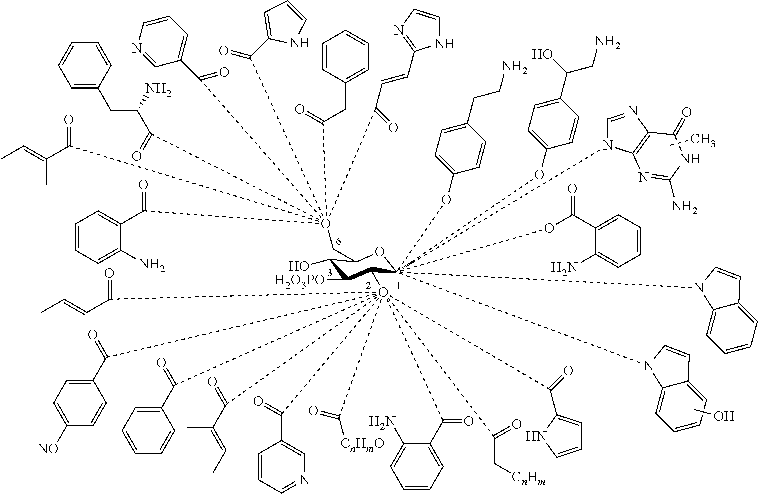

For avoidance of the doubt, the depiction above represents the combinatorial range of unique molecules resulting from independently choosing any one of the depicted moieties at each of the indicated positions by replacement of a dashed line in the figure with a covalent bond. The family of molecules represented by this depiction (and other similar depictions herein) includes all combinatorial permutations resulting from independent selection of each moiety at each position.

In certain embodiments, provided therapeutic compositions comprise MOGLs featuring a glucose derivative substituted at the 1-, 2-, and 3-positions, wherein:
the substituent at the 1-position is selected from the group consisting of a nucleobase, an N-linked heterocycle, an acyl- or ether-linked aromatic moiety an ether-linked aromatic moiety substituted with an amine or aminoalkyl group;
the substituent at the 2-position comprises an optionally substituted aromatic, heteroaromatic, or aliphatic acyl group; and
the substituent at the 3-position comprises a phosphate, diphosphate, triphosphate, or a derivative of any of these.
in certain embodiments, the provided compositions are further characterized in that the glucose derivative is not substituted at the 4- or 6-position.

In certain embodiments, such compositions comprise a therapeutically effective amount of one or more compounds of Formula II:

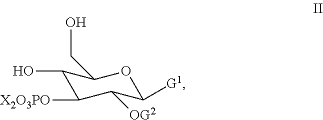

wherein each of G¹, G² and X is as defined above and in the genera and subgenera herein.

In certain embodiments, a compound of Formula II comprises any of the modular glucosides encompassed by the formula:

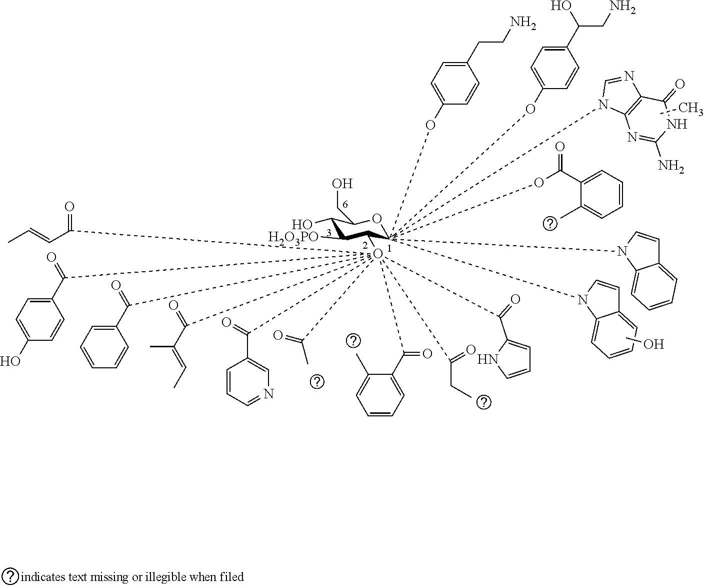

In certain embodiments, the present invention provides therapeutic compositions comprising MOGLs featuring a glucose derivative substituted at the 1-, 3-, and 6-positions, wherein:
the substituent at the 1-position is selected from the group consisting of a nucleobase, an N-linked heterocycle, an acyl- or ether-linked aromatic moiety an ether-linked aromatic moiety substituted with an amine or aminoalkyl group;
the substituent at the 3-position comprises a phosphate, diphosphate, triphosphate, or a derivative of any of these; and
the substituent at the 6-position comprises an acyl-linked amino acid, or an optionally substituted aromatic or aliphatic acyl group;

In certain embodiments, the provided compositions are further characterized in that the glucose derivative is not substituted at the 2- or 4-position

In certain embodiments, such therapeutic compositions comprise a therapeutically effective amount of one or more compounds of Formula III:

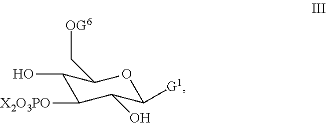

wherein each of G¹, G⁶ and X is as defined above and in the genera and subgenera herein.

In certain embodiments, a compound of Formula III comprises any of the modular glucosides encompassed by the formula:

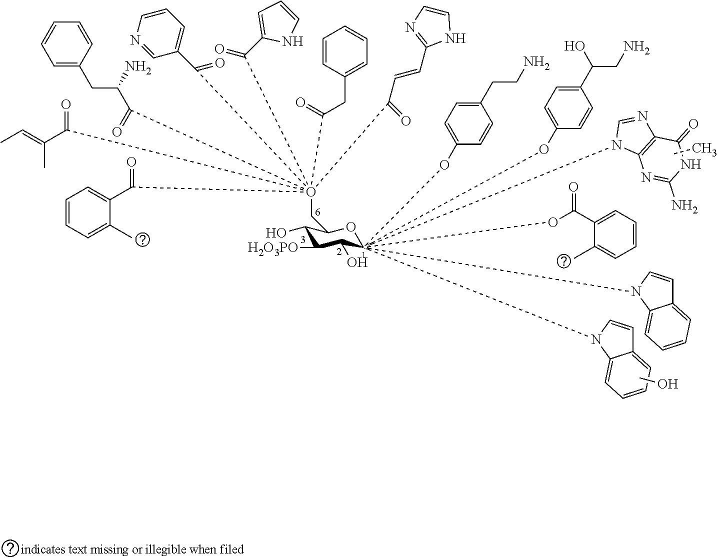

In certain embodiments, such therapeutic compositions comprise MOGLs featuring a glucose derivative substituted at the 1- and 3-positions, wherein:
the substituent at the 1-position is selected from the group consisting of a nucleobase, an N-linked heterocycle, an acyl- or ether-linked aromatic moiety an ether-linked aromatic moiety substituted with an amine or aminoalkyl group;
the substituent at the 3-position comprises a phosphate, diphosphate, triphosphate, or a derivative of any of these; and

In certain embodiments, the provided compositions are further characterized in that the glucose derivative is not substituted at the 2-, 4- or 6-position.

In certain embodiments, the present invention encompasses compositions of matter comprising a therapeutically effective amount of one or more compounds of Formula IV:

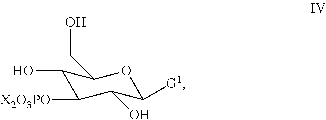

wherein each of G¹ and X is as defined above and in the genera and subgenera herein.

In certain embodiments, a compound of Formula IV comprises any of the modular glucosides encompassed by the formula:

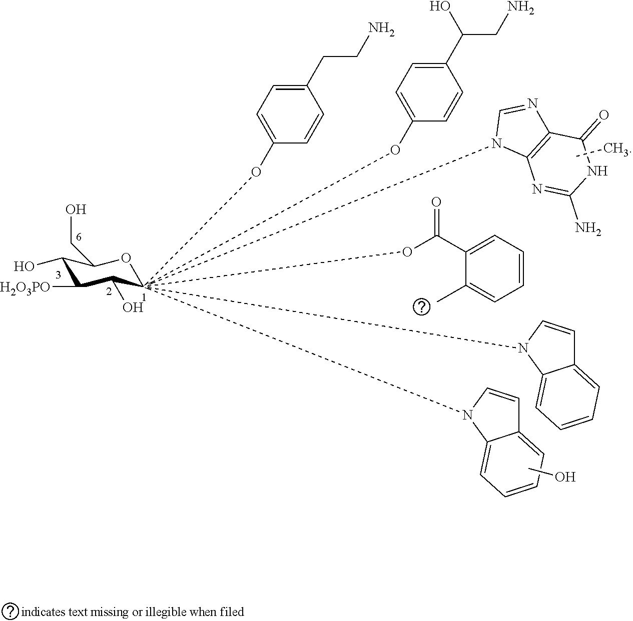

In certain embodiments, the present invention provides therapeutic compositions comprising MOGLs featuring a glucose derivative substituted at the 1-, 2-, and 6-positions, wherein:
the substituent at the 1-position is selected from the group consisting of a nucleobase, an N-linked heterocycle, an acyl- or ether-linked aromatic moiety an ether-linked aromatic moiety substituted with an amine or aminoalkyl group;
the substituent at the 2-position comprises an optionally substituted aromatic, heteroaromatic, or aliphatic acyl group;
the substituent at the 6-position comprises an acyl-linked amino acid, or an optionally substituted aromatic or aliphatic acyl group;

In certain embodiments, the provided compositions are further characterized in that the glucose derivative is not substituted at the 3- or 4-position.

In certain embodiments, such compositions comprise a therapeutically effective amount of one or more compounds of Formula V:

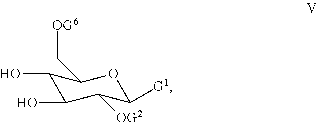

wherein each of G¹, G² and G⁶ is as defined above and in the genera and subgenera herein.

In certain embodiments, a compound of Formula V comprises any of the modular glucosides encompassed by the formula:

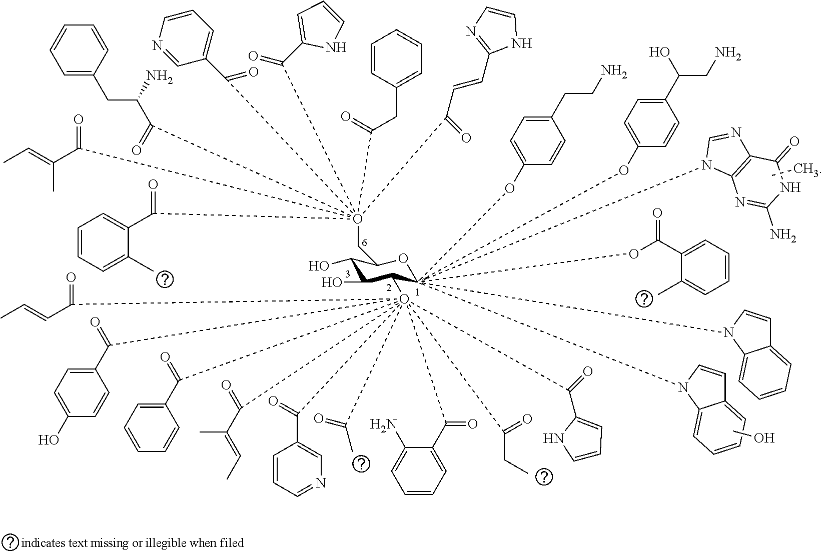

In certain embodiments, the present invention provides therapeutic compositions comprising MOGLs featuring a glucose derivative substituted at the 1- and 2-, positions, wherein:
the substituent at the 1-position is selected from the group consisting of a nucleobase, an N-linked heterocycle, an acyl- or ether-linked aromatic moiety an ether-linked aromatic moiety substituted with an amine or aminoalkyl group;
the substituent at the 2-position comprises an optionally substituted aromatic, heteroaromatic, or aliphatic acyl group;

In certain embodiments, the provided compositions are further characterized in that the glucose derivative is not substituted at the 3-, 4- or 6-position.

In certain embodiments, such compositions comprise a therapeutically effective amount of one or more compounds of Formula VI:

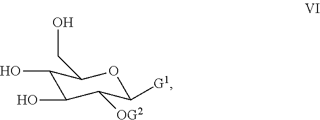

wherein each of G¹ and G² is as defined above and in the genera and subgenera herein.

In certain embodiments, a compound of Formula VI comprises any of the modular glucosides encompassed by the formula:

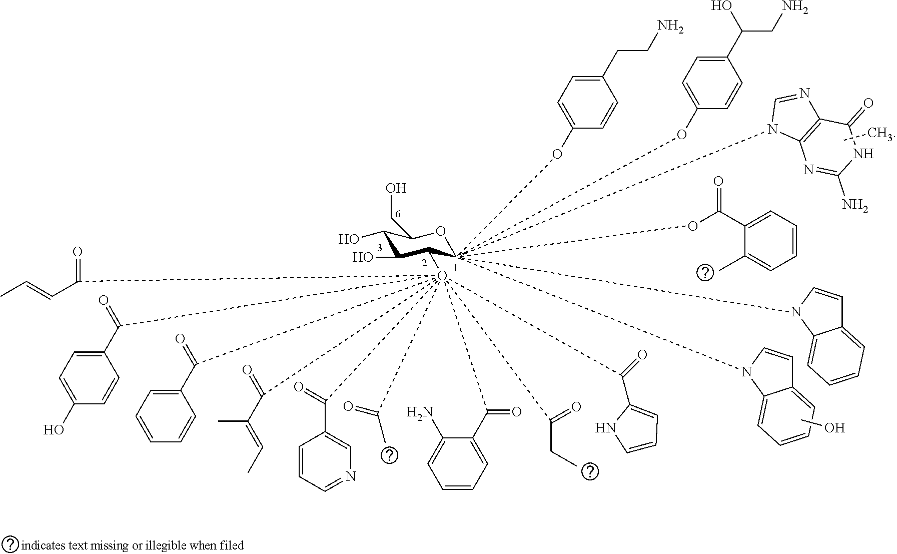

In certain embodiments, the present invention provides therapeutic compositions comprising MOGLs featuring a glucose derivative substituted at the 1- and 6-positions, wherein:
the substituent at the 1-position is selected from the group consisting of a nucleobase, an N-linked heterocycle, an acyl- or ether-linked aromatic moiety an ether-linked aromatic moiety substituted with an amine or aminoalkyl group;
the substituent at the 6-position comprises an acyl-linked amino acid, or an optionally substituted aromatic or aliphatic acyl group;

In certain embodiments, the provided compositions are further characterized in that the glucose derivative is not substituted at the 2-, 3- or 4-position.

In certain embodiments, such compositions comprise a therapeutically effective amount of one or more compounds of Formula VII:

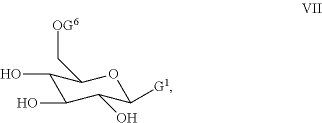

wherein each of G¹, and G⁶ is as defined above and in the genera and subgenera herein.

In certain embodiments, a compound of Formula VII comprises any of the modular glucosides encompassed by the formula:

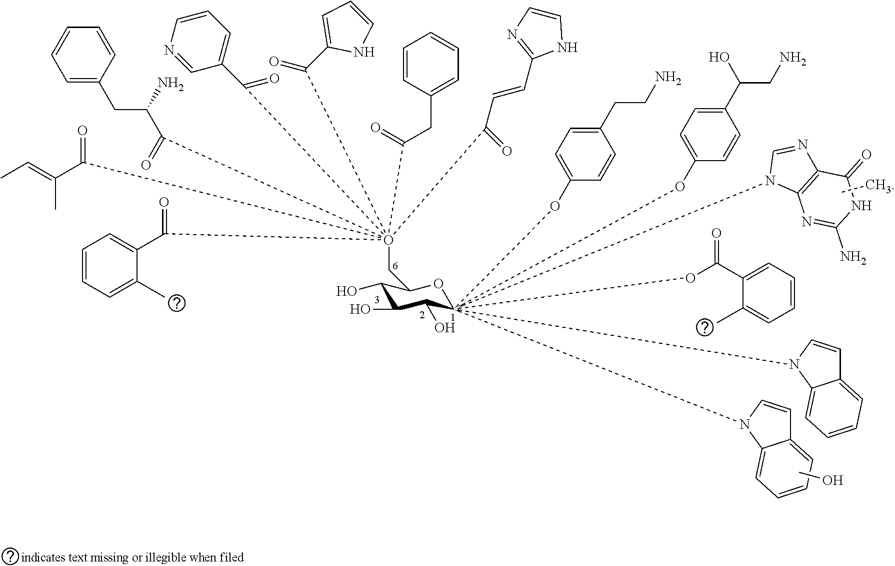

In certain embodiments, the MOGL-containing therapeutic compositions described above are characterized in that the substituent at the 1-position comprises a nucleobase. In certain embodiments, the nucleobase is N-linked to the 1-position of the glucose scaffold. In certain embodiments, the N-linked nucleobase comprises a pyrimidine base. In certain embodiments, the N-linked nucleobase comprises a purine base. In certain embodiments, the N-linked nucleobase comprises a primary nucleobase. In certain embodiments, the N-linked nucleobase is other than a primary nucleobase, or is an analog or adduct of a primary nucleobase. In certain embodiments, the N-linked nucleobase is a methylated nucleobase. In certain embodiments, the N-linked nucleobase is selected from the group consisting of adenine, cytosine, guanine, thymine, and uracil. In certain embodiments, the nucleobase comprises guanine. In certain embodiments, the nucleobase comprises a methylated analog of guanine. In certain embodiments, the nucleobase comprises 6-O-methyl guanine.

In certain embodiments, the MOGL-containing therapeutic compositions described above are characterized in that the substituent at the 1-position comprises an optionally substituted N-linked heterocycle. In certain embodiments, the N-linked heterocycle comprises a 5- or 6-membered ring containing at least one nitrogen atom. In certain embodiments, the N-linked heterocycle is optionally substituted 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur. In certain embodiments, the N-linked heterocycle contains one or more sites of unsaturation. In certain embodiments, the N-linked heterocycle comprises indole. In certain embodiments, the N-linked heterocycle comprises a substituted indole. In certain embodiments, the N-linked heterocycle comprises a hydroxy indole. In certain embodiments, the N-linked heterocycle comprises serotonin.

In certain embodiments, the MOGL-containing therapeutic compositions described above are characterized in that the substituent at the 1-position comprises an optionally unsaturated acyl group. In certain embodiments, the substituent at the 1-position comprises an alpha beta unsaturated acyl group. In certain embodiments, an acyl substituent at the 1-position comprises a C₃₋₈ aliphatic group with alpha beta unsaturation. In certain embodiments, the substituent at the 1-position comprises crotonate. In certain embodiments, the substituent at the 1-position comprises tiglate. In certain embodiments, the substituent at the 1-position comprises angelate. In certain embodiments, the substituent at the 1-position comprises valerate. In certain embodiments, the substituent at the 1-position comprises acrylate, methacrylate, or cinnamate. In certain embodiments, the substituent at the 1-position comprises 2-imidazoleacrylate. In certain embodiments, the substituent at the 1-position comprises urocanate.

In certain embodiments, the MOGL-containing therapeutic compositions described above are characterized in that the substituent at the 1-position comprises an acyl- or ether-linked aromatic moiety substituted with an amine or an aminoalkyl group. In certain embodiments, the provided compositions are characterized in that the substituent at the 1-position comprises an acyl-linked aromatic moiety substituted with an amine. In certain embodiments, the acyl-linked aromatic moiety comprises a phenyl ring. In certain embodiments, the acyl-linked aromatic moiety comprises a substituted benzoyl group. In certain embodiments, the acyl-linked aromatic moiety comprises an optionally substituted aminobenzoyl group. In certain embodiments, the acyl-linked aromatic moiety comprises anthranilic acid. In certain embodiments, substituent at the 1-position comprises an ether-linked aromatic moiety substituted with an aminoalkyl group. In certain embodiments, the ether-linked aromatic moiety comprises a phenyl ring. In certain embodiments, a substituent at the 1-position comprises a phenolic ether where the phenyl ring of the phenol is substituted with an aminoalkyl group. In certain embodiments, the ether-linked aromatic moiety comprises a phenol substituted with an optionally substituted 2-aminoethyl group. In certain embodiments, the ether-linked aromatic moiety comprises tyramine. In certain embodiments, the ether-linked aromatic moiety comprises octopamine. In certain embodiments the the substituent at the 1-position comprises O-linked serotonin. In certain embodiments the the substituent at the 1-position comprises O-linked N-acetylserotonin (normelatonin). In certain embodiments the the substituent at the 1-position comprises O-linked dopamine. In certain embodiments the the substituent at the 1-position comprises 3-O-linked dopamine. In certain embodiments the the substituent at the 1-position comprises 4-O-linked dopamine. In certain embodiments the the substituent at the 1-position comprises 3-O-linked norepinepherine. In certain embodiments the the substituent at the 1-position comprises 4-O-linked norepinepherine. In certain embodiments the the substituent at the 1-position comprises 3-O-linked epinepherine. In certain embodiments the the substituent at the 1-position comprises 4-O-linked epinepherine.

In some embodiments, G¹ in any of Formulae I, II, III, IV, V, VI, or VII above is selected from:

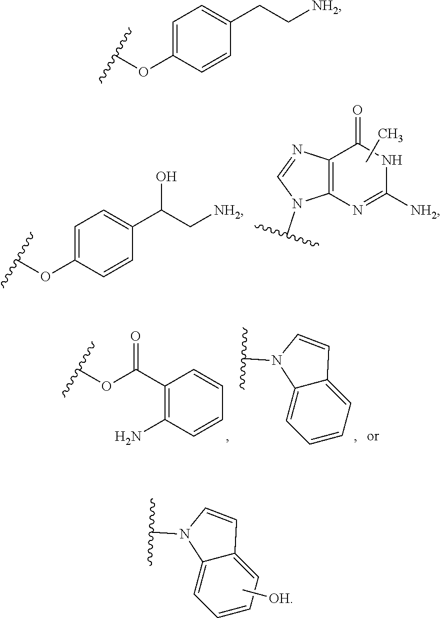

In some embodiments, G¹ in any of Formulae I, II, III, IV, V, VI, or VII above is selected from:

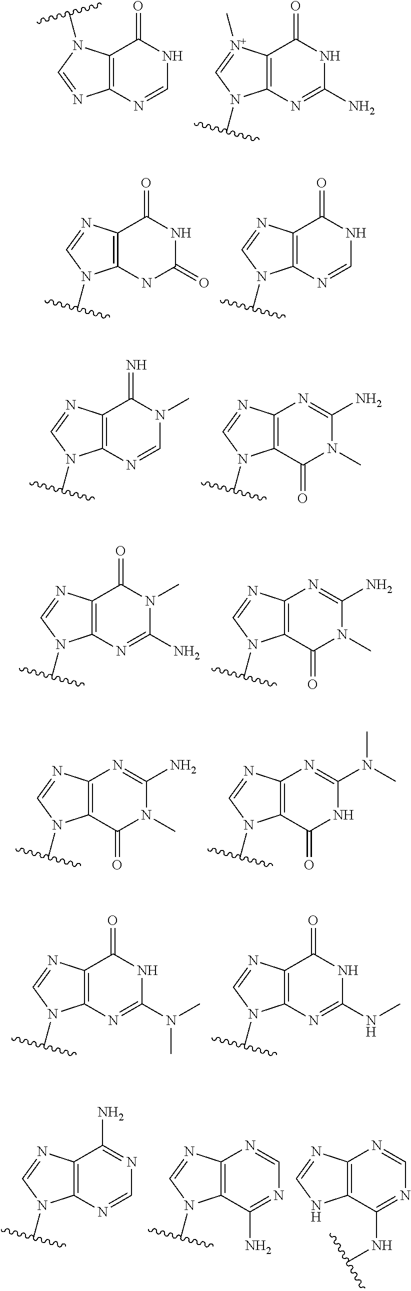

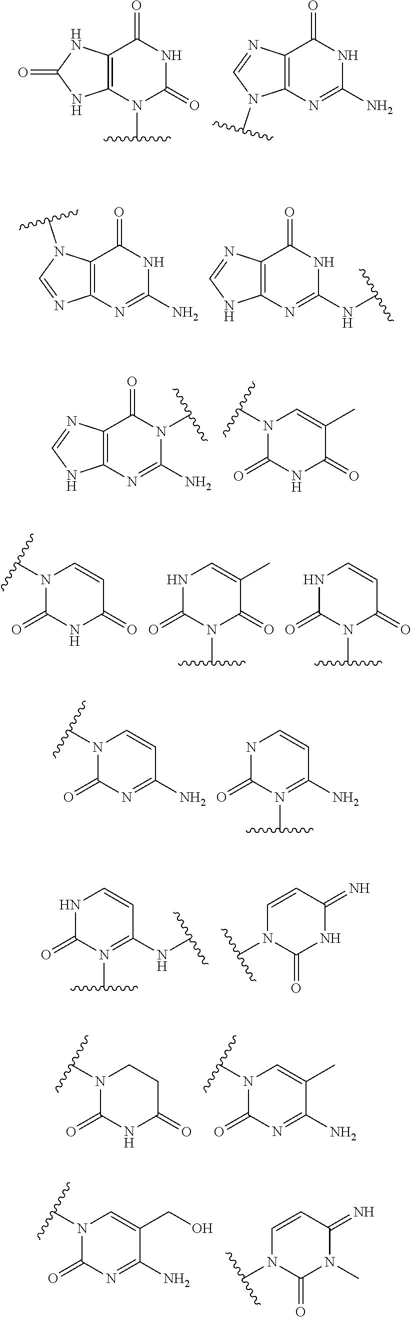

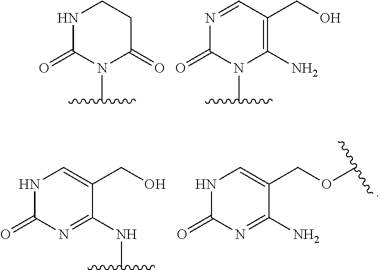

In some embodiments, G¹ in any of Formulae I, II, III, IV, V, VI, or VII above is selected from:

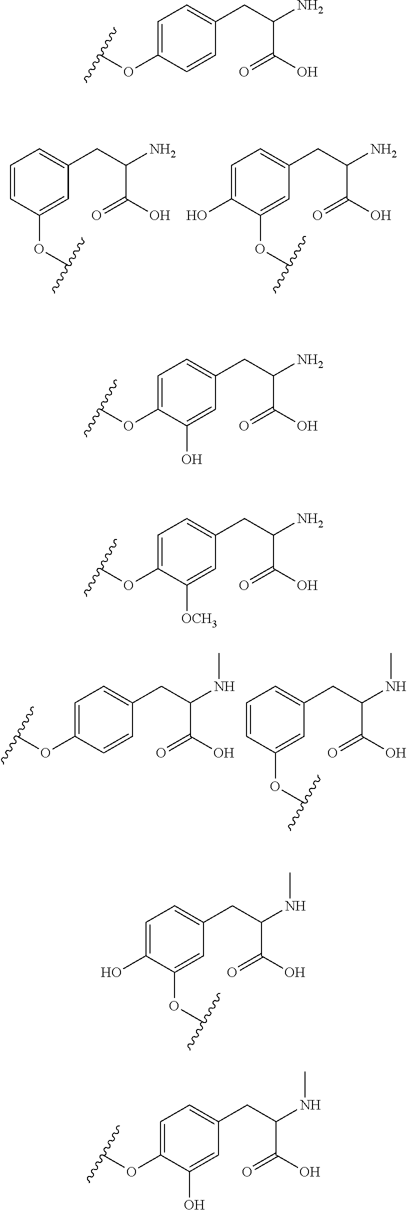

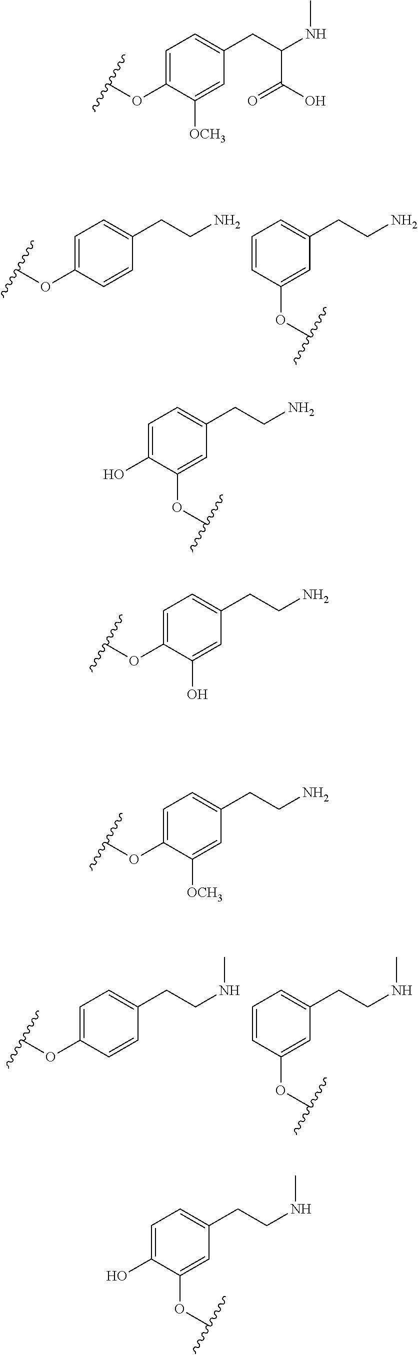

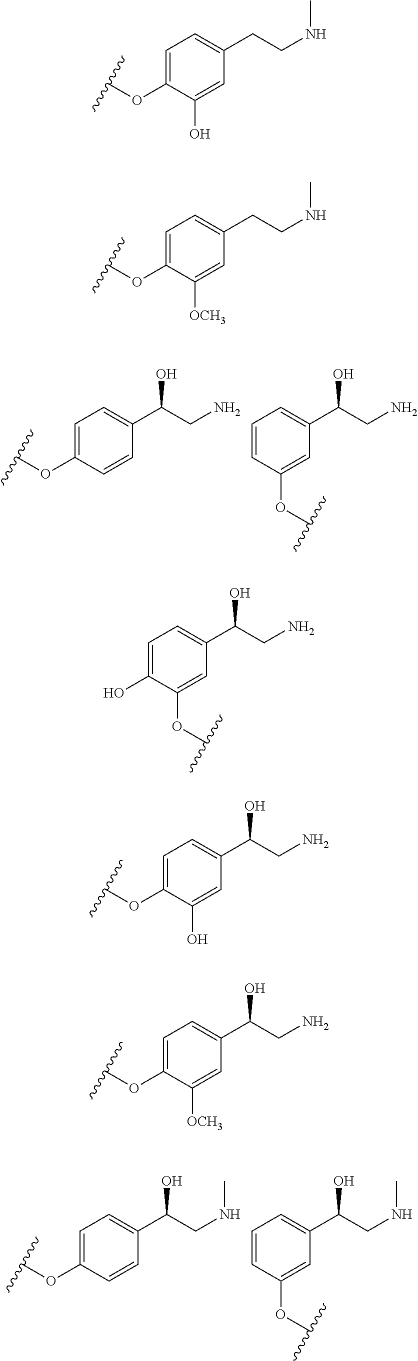

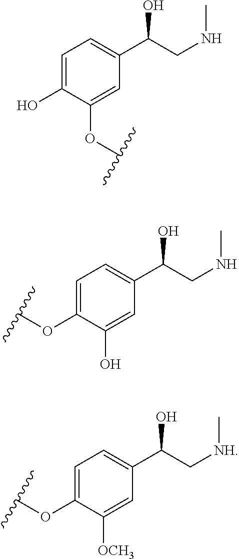 [SMILES: *Oc1cc([C@@H](O)CNC)ccc1O ; *Oc1ccc([C@@H](O)CNC)cc1O ; *Oc1ccc([C@@H](O)CNC)cc1OC]

In some embodiments, G¹ in any of Formulae I, II, III, IV, V, VI, or VII above is selected from:

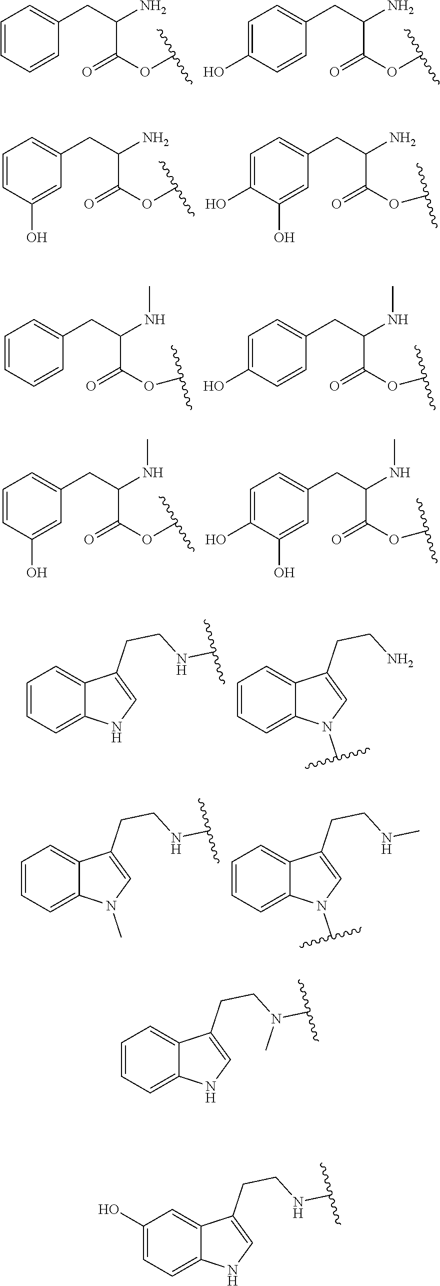

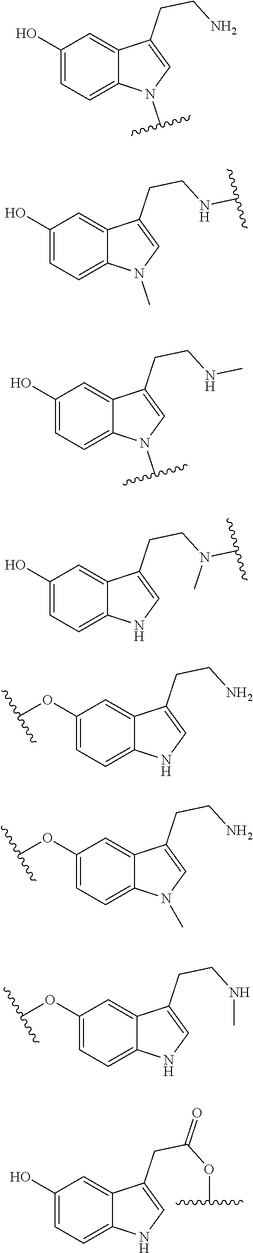

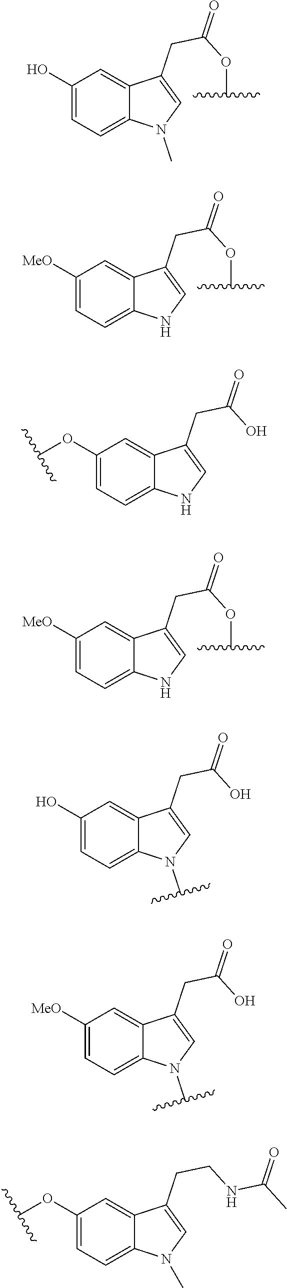

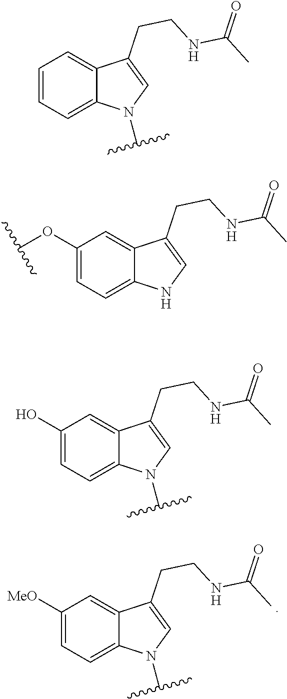 [SMILES: *n1cc(CCNC(C)=O)c2ccccc21 ; *Oc1ccc2[nH]cc(CCNC(C)=O)c2c1 ; *n1cc(CCNC(C)=O)c2cc(O)ccc21 ; *n1cc(CCNC(C)=O)c2cc(OC)ccc21]

In certain embodiments, therapeutic compositions of the present invention comprise a MOGL having a neurotransmitter (or a derivative or precursor of a neurotransmitter) linked to the 1-position of the glucose. In certain embodiments, such compositions comprise one or more compounds selected from the group:

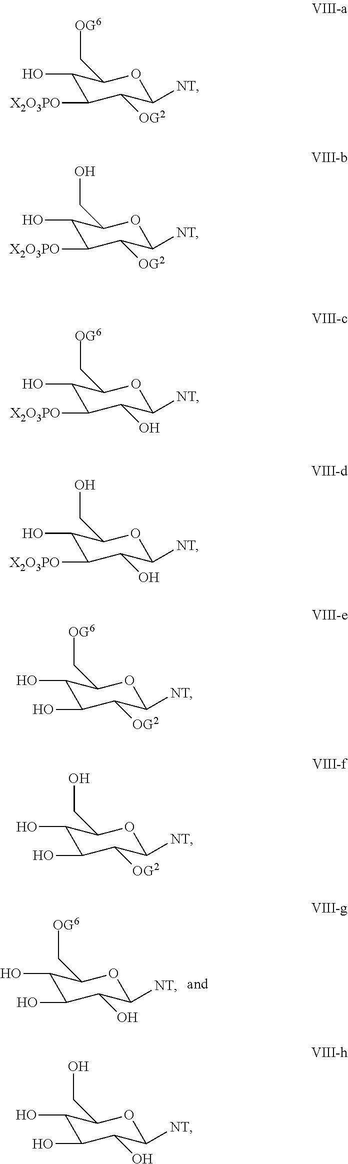

where each of G², G³, G⁶, and X is as defined above and in the genera and subgenera herein, and —NT comprises a neurotransmiltter, or a derivative or precursor of a neurotransmitter linked to the glucose through any suitable atom.

In certain embodiments, the neurotransmitter is linked to the glucose through a nitrogen or oxygen atom comprising part of the neurotransmitter structure. In some embodiments, the neurotransmitter is linked to the glucose through an atom from which a hydrogen is removed, with the resulting radical forming the point of attachment. In certain embodiments, the neurotransmitter is N-linked. In certain embodiments a neurotransmiltter is linked via a phenolic oxygen. In certain embodiments a neurotransmitter is linked via an acyl linkage.

In certain embodiments, the moiety —NT comprises a monoamine neurotransmitter or a derivative or precursor thereof. In certain embodiments, —NT comprises a catecholamine neurotransmitter or a derivative or precursor thereof. In certain embodiments —NT is selected from the group consisting of: dopamine, norepinepherine, epinepherine, histamine, and serotonin. In certain embodiments, —NT is selected from the group consisting of dopamine, norepinepherine, and epinepherine. In certain embodiments, —NT is selected from tryptamine, phenethylamine, N-methylphenethylamine, phenethanolamine, m-tyramine, p-tyramine, 3-methoxytyramine, N-methyltyramine, 3-indothyronamine, m-octopamine, p-octopamine, and synepherine.

In certain embodiments, the moiety —NT is selected from the group consisting of:

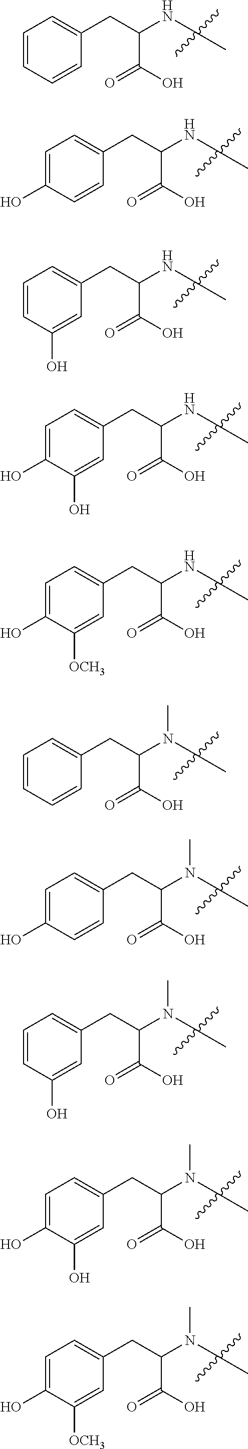

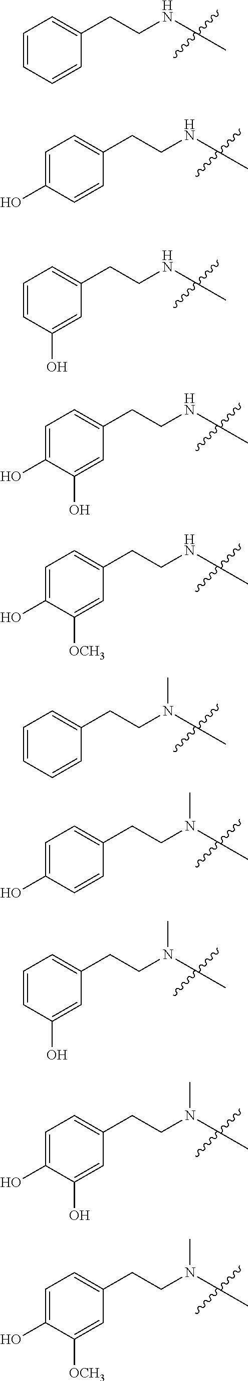

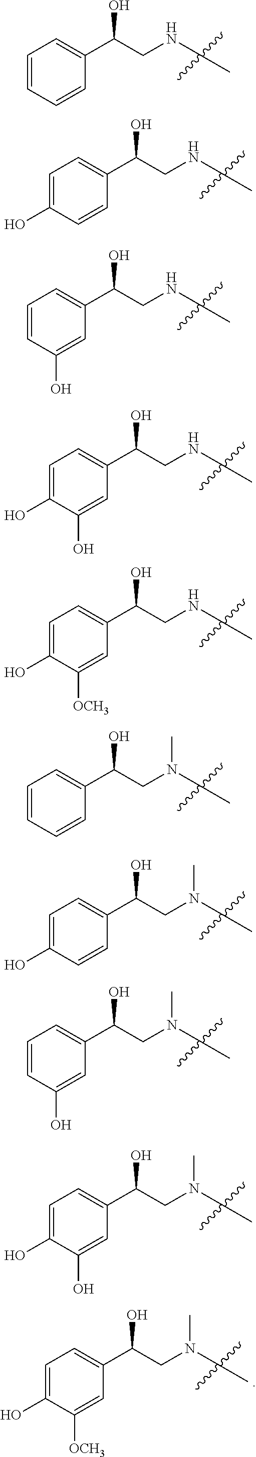

In certain embodiments, the moiety —NT is selected from the group consisting of

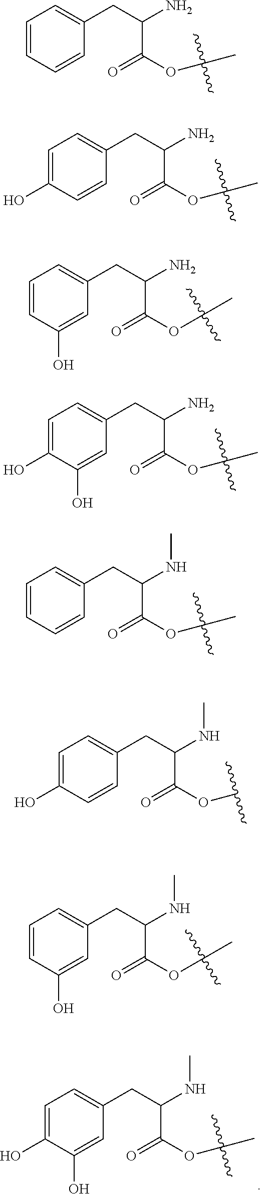

In certain embodiments, the moiety —NT is selected from the group consisting of

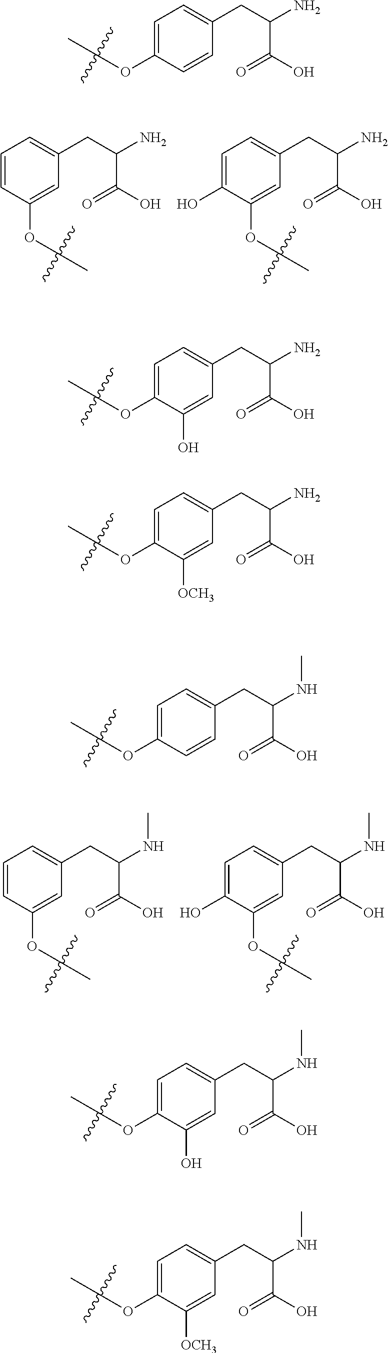

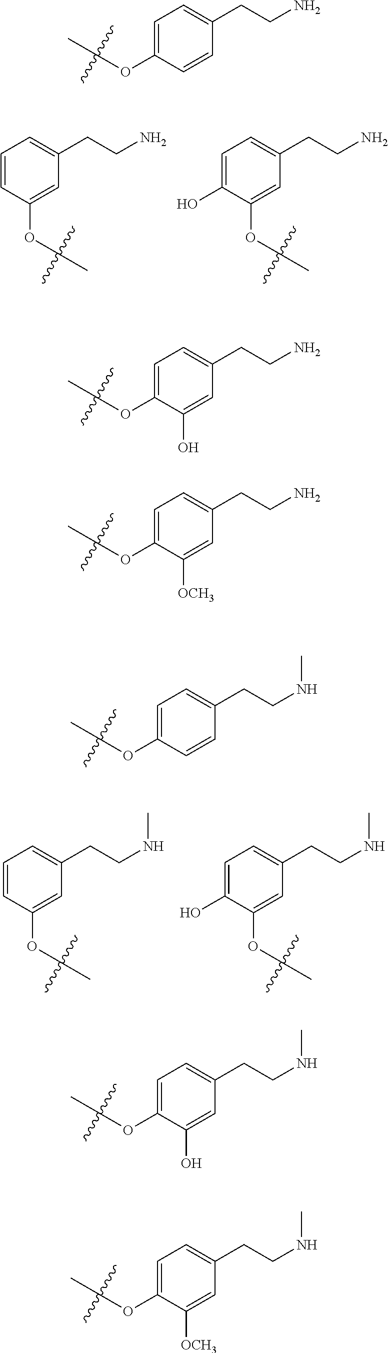

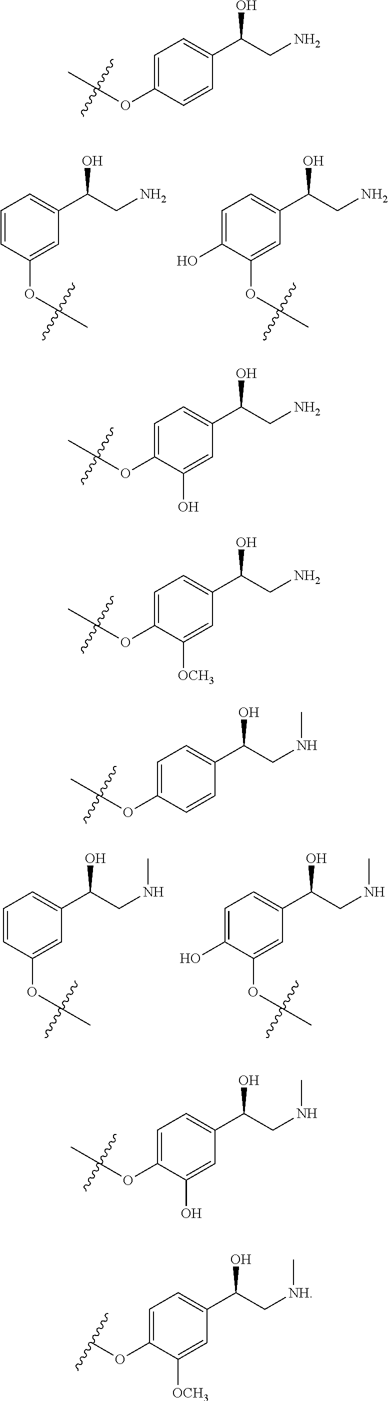

In certain embodiments, the moiety —NT is selected from the group consisting of

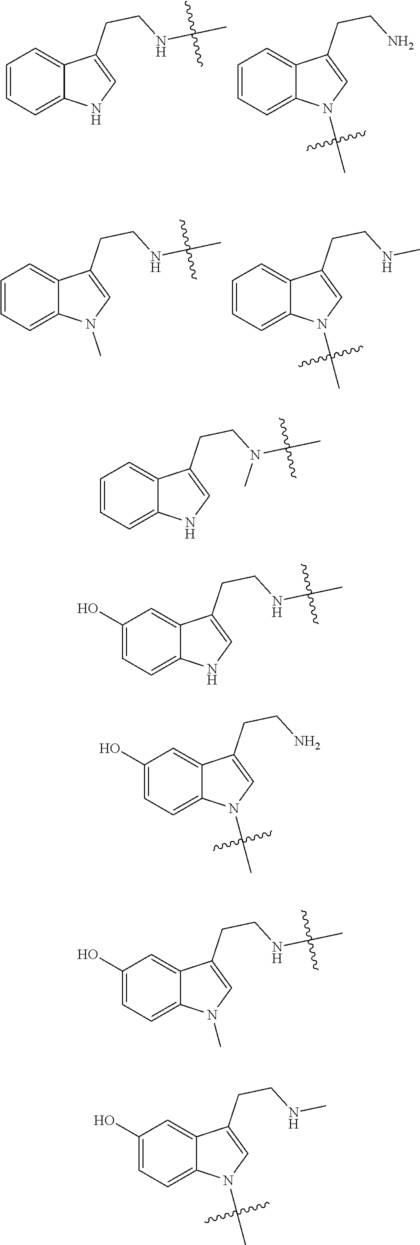

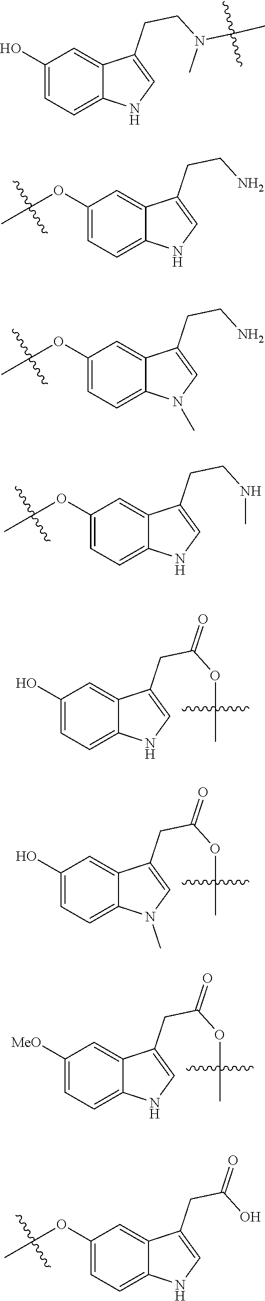

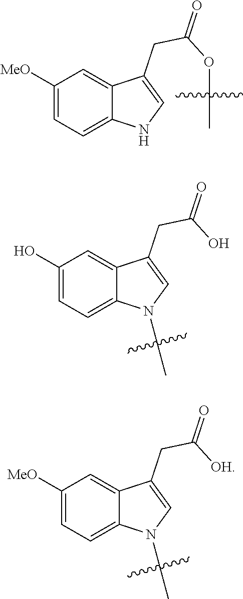 [SMILES: *OC(=O)Cc1c[nH]c2ccc(OC)cc12 ; *n1cc(CC(=O)O)c2cc(O)ccc21 ; *n1cc(CC(=O)O)c2cc(OC)ccc21]

In certain embodiments, therapeutic compositions of the present invention comprise a MOGL having a nucleobase (or a derivative or precursor of a nucleobase) linked to the 1-position of the glucose. In certain embodiments, such compositions comprise one or more compounds selected from the group:

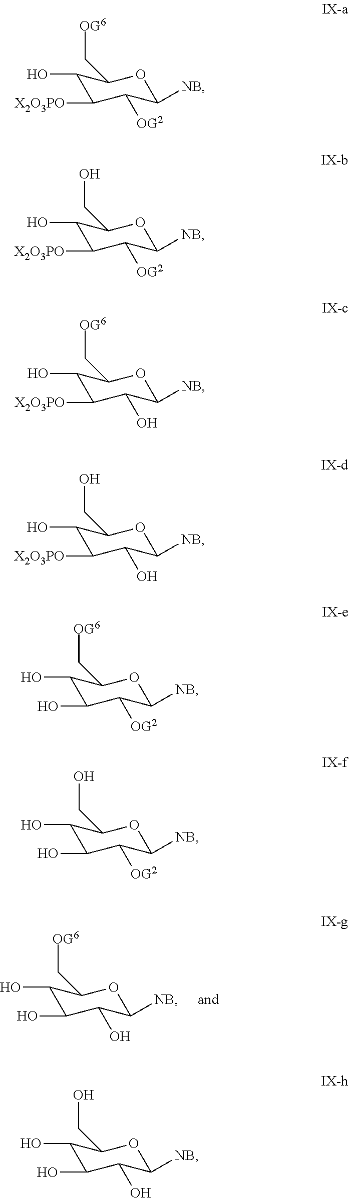

where each of G2, G3, G6, and X is as defined above and in the genera and subgenera herein, and
—NB comprises an aromatic moiety, a nucleobase, or a derivative or precursor of a nucleobase linked to the glucose through any suitable atom.

In certain embodiments, the nucleobase is linked to the glucose through a nitrogen or oxygen atom comprising part of the nucleobase structure. In some embodiments, the nucleobase is linked to the glucose through an atom from which a hydrogen is removed, with the resulting radical forming the point of attachment. In certain embodiments, the nucleobase is N-linked.

In certain embodiments, the moiety —NB is selected from the group consisting of:

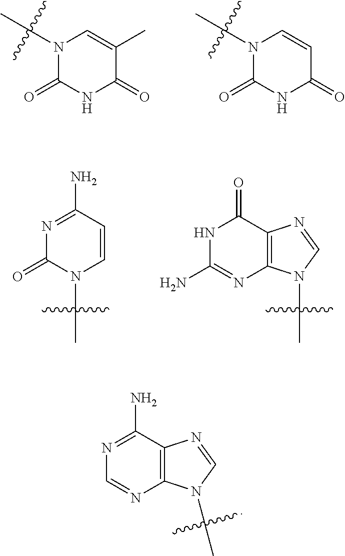

In certain embodiments, the moiety —NB is selected from the group consisting of:

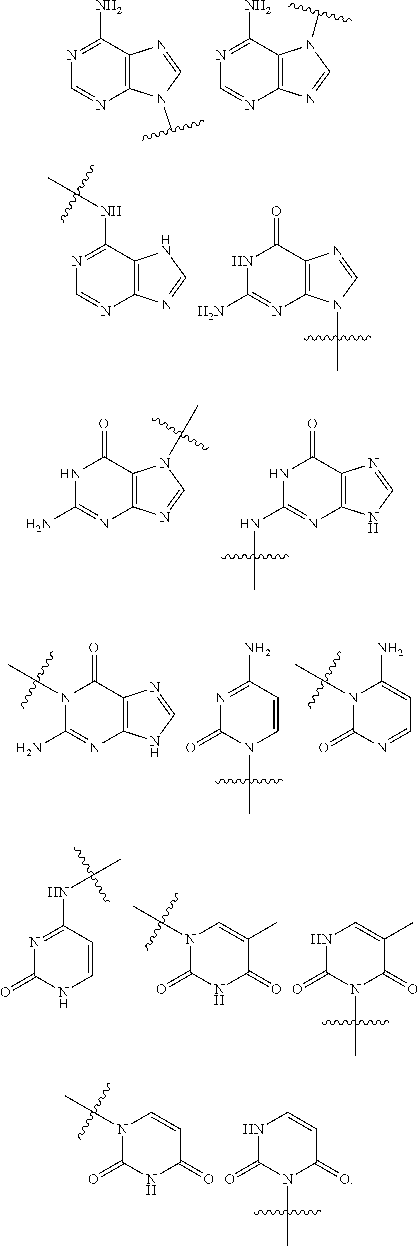

In certain embodiments, the moiety —NB is selected from the group consisting of:

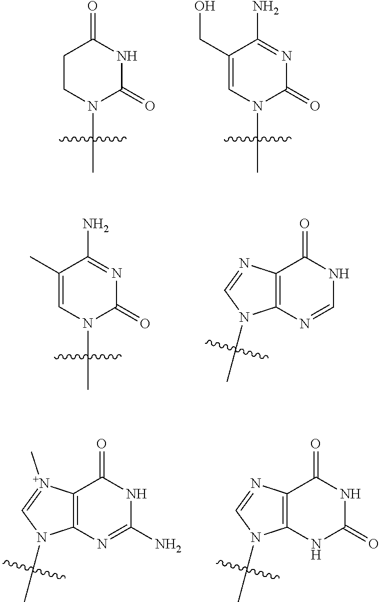

In certain embodiments, the moiety —NB is selected from the group consisting of:

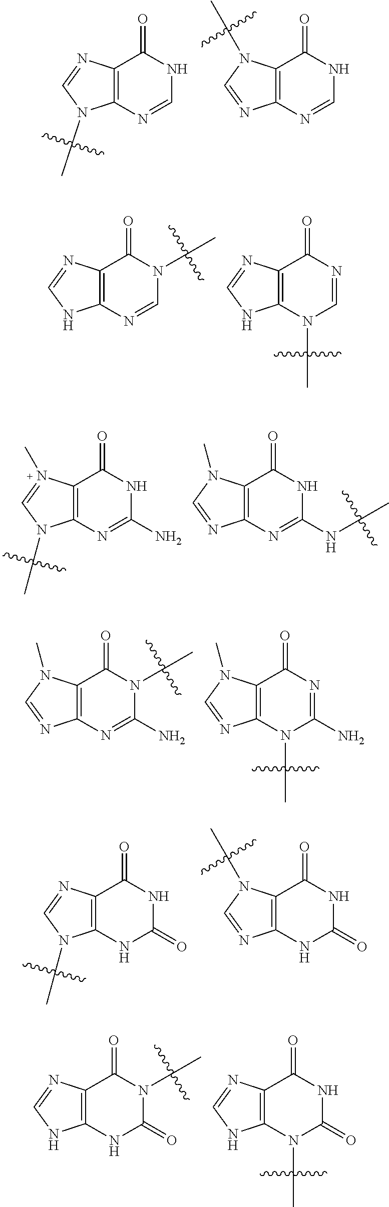

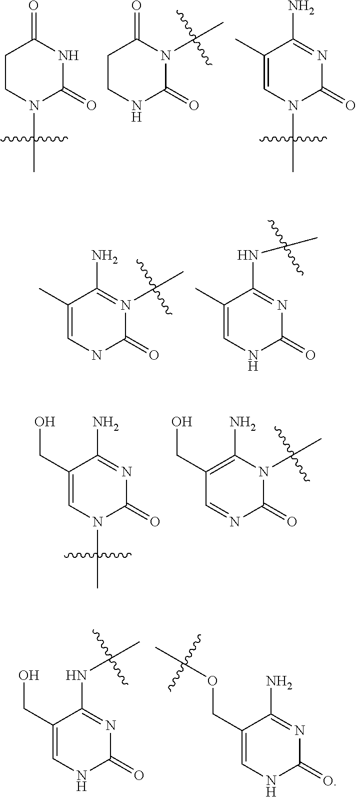

In certain embodiments, therapeutic compositions of the present invention comprise a MOGL having an alpha beta unsaturated acyl group linked to the 1-position of the glucose. In certain embodiments, such compositions comprise one or more compounds selected from the group:

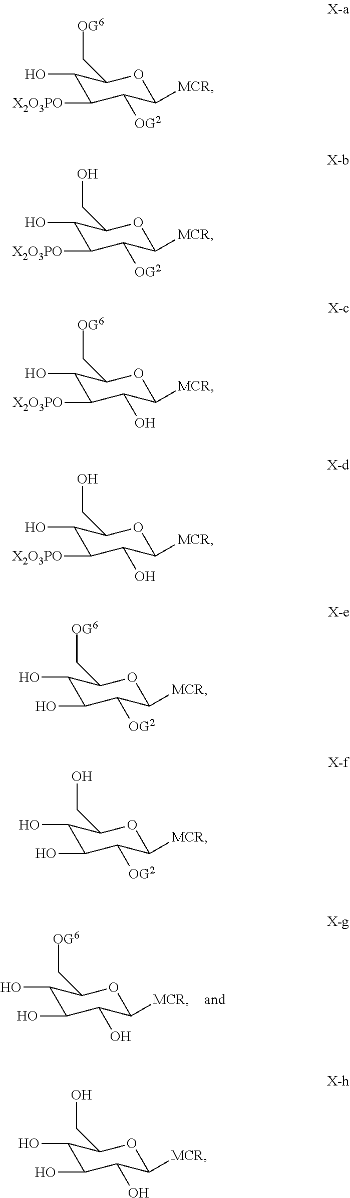

where each of G², G³, G⁶, and X is as defined above and in the genera and subgenera herein, and -MCR comprises a C₃₋₁₂ alpha beta unsaturated acyl group.

In certain embodiments, the moiety -MCR comprises a C₃₋₈ alpha beta unsaturated acyl group. In certain embodiments, the moiety -MCR comprises a C₄₋₈ alpha beta unsaturated acyl group. In certain embodiments, the moiety -MCR comprises an acyl group corresponding to an ester of acrylic acid, methylacrylic acid, crotonic acid, methyl crotonic acid, valeric acid, 3-methylcrotonic acid or tiglic acid. In certain embodiments, the substituent at the 1-position comprises crotonate. In certain embodiments, the moiety -MCR comprises crotonate. In certain embodiments, the moiety -MCR comprises tiglate. In certain embodiments, the moiety MCR comprises angelate. In certain embodiments, the moiety -MCR comprises valerate. In certain embodiments, the moiety -MCR comprises acrylate, methacrylate, or cinnamate. In certain embodiments, the moiety MCR comprises 2-imidazoleacrylate. In certain embodiments, the moiety -MCR comprises urocanate.

In certain embodiments, the MOGL-containing therapeutic compositions described above are characterized in that the substituent at the 2-position comprises an optionally substituted aromatic or heteroaromatic acyl group. In certain embodiments, the substituent at the 2-position comprises an optionally substituted benzoate. In certain embodiments the optionally substituted benzoate is selected from the group consisting of: benzoate, anthranilate, and p-hydroxybenzoate. In certain embodiments, the substituent at the 2-position comprises an optionally substituted heteroaromatic acyl group. In certain embodiments, the substituent at the 2-position comprises a heteroaromatic acyl group with a 6-membered heteroaromatic moiety. In certain embodiments, the substituent at the 2-position comprises a pyridine or pyrimidine carboxylate ester. In certain embodiments, the 2-substituent comprises nicotinate. In certain embodiments, the 2-substituent comprises picolinate. In certain embodiments, the 2-substituent comprises isonicotinate. In certain embodiments, the substituent at the 2-position comprises a heteroaromatic acyl group with a 5-membered heteroaromatic moiety. In certain embodiments, the substituent at the 2-position comprises the ester of a pyrrole or imidazole carboxylic acid. In certain embodiments, the substituent at the 2-position is pyrrole-2-carboxylate.

In certain embodiments, the MOGL-containing therapeutic compositions described above are characterized in that the substituent at the 2-position comprises an optionally substituted aliphatic acyl group. In certain embodiments, the acyl group at the 2-position comprises an optionally substituted aliphatic group. In certain embodiments, the acyl group at the 2-position comprises an optionally substituted C₁₋₄₀ aliphatic group, an optionally substituted C₂₋₂₄ aliphatic group, an optionally substituted C₁₂₋₂₄ aliphatic group, an optionally substituted C₂₋₁₈ aliphatic group, an optionally substituted C₂₋₁₂ aliphatic group, an optionally substituted C₂₋₈ aliphatic group, or an optionally substituted C₁₋₆ aliphatic group. In certain embodiments, the acyl group at the 2-position comprises a hydroxylated C₁₋₄₀ aliphatic group. In certain embodiments, the acyl group at the 2-position comprises an epoxidized substituted C₁₋₄₀ or C₂₋₄₀ aliphatic group. In certain embodiments, such optionally substituted aliphatic groups are saturated. In certain embodiments, optionally substituted aliphatic groups have one or more sites of unsaturation. In certain embodiments, such unsaturated aliphatic groups have unsaturation adjacent to the carbonyl of the acyl linkage (e.g. they are alpha-beta unsaturated esters). In certain embodiments, an acyl substituent at the 2-position comprises a C₂₋₈ aliphatic group with alpha beta unsaturation. In certain embodiments, the substituent at the 2-position comprises crotonate. In certain embodiments, the substituent at the 2-position comprises tiglate. In certain embodiments, the substituent at the 2-position comprises angelate. In certain embodiments, the substituent at the 2-position comprises acrylate, methacrylate, 3-methylcrotonate, isocrotonate, or optionally substituted cinnamate.

In some embodiments, the present invention provides a compound of Formulae XI-a, XI-b, XI-c, XI-d, XI-e, XI-f, or XI-g:

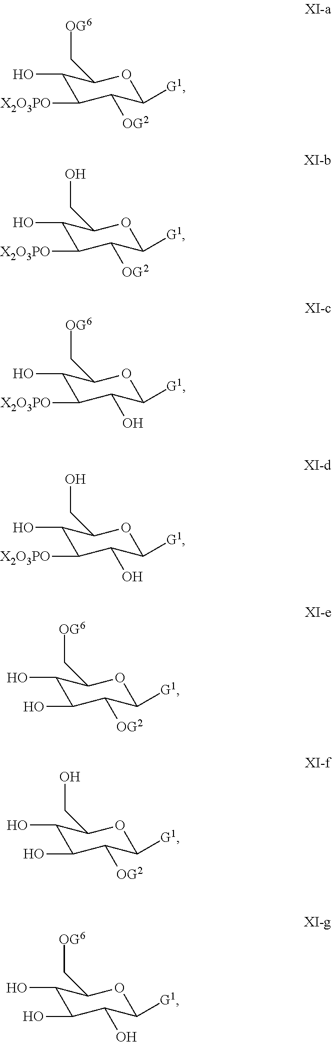

or a pharmaceutically acceptable salt thereof, wherein each of G², G⁶, and X is as defined above and described in classes and subclasses herein, both singly and in combination, and wherein:
G¹ is —NRⁿ¹Rⁿ², wherein Rⁿ¹ and Rⁿ² are each independently selected from the group consisting of hydrogen, optionally substituted C₁₋₂₀ aliphatic, optionally substituted C₁₋₂₀ acyl, optionally substituted aryl, and optionally substituted heterocyclic.

In some embodiments, Rⁿ¹ is hydrogen and Rⁿ² is optionally substituted C₁₋₁₂ aliphatic. In some embodiments, Rⁿ¹ is hydrogen and Rⁿ² is optionally substituted C₁₋₆ aliphatic. In some embodiments, Rⁿ¹ is methyl and Rⁿ² is optionally substituted C₁₋₆ aliphatic. In some embodiments, Rⁿ¹ is hydrogen and Rⁿ² is optionally substituted aryl. In some embodiments, Rⁿ¹ is hydrogen and Rⁿ² is optionally substituted aryl. In some embodiments, Rⁿ¹ is methyl and R² is optionally substituted aryl. In some embodiments, Rⁿ¹ is hydrogen and Rⁿ² is optionally substituted C₃₋₁₂ heterocyclic. In some embodiments, Rⁿ¹ is hydrogen and Rⁿ² is optionally substituted C₃₋₁₂ heterocyclic. In some embodiments, Rⁿ¹ is methyl and Rⁿ² is optionally substituted C₃₋₆ heterocyclic. In some embodiments, Rⁿ¹ is hydrogen and Rⁿ² is optionally substituted acyl. In some embodiments, Rⁿ¹ is hydrogen and Rⁿ² is optionally substituted acyl.

In some embodiments, Rⁿ¹ is methyl and Rⁿ² is optionally substituted acyl. In some embodiments, —NRⁿ¹Rⁿ² comprises a monoamine neurotransmitter or a derivative or precursor thereof. In certain embodiments, —NRⁿ¹Rⁿ² comprises a catecholamine neurotransmitter or a derivative or precursor thereof. In certain embodiments —NRⁿ¹Rⁿ² is selected from the group consisting of: dopamine, norepinepherine, epinepherine, histamine, and serotonin. In certain embodiments, —NRⁿ¹Rⁿ² is selected from the group consisting of dopamine, norepinepherine, and epinepherine. In certain embodiments, —NRⁿ¹Rⁿ² is selected from tryptamine, phenethylamine, N-methylphenethylamine, phenethanolamine, m-tyramine, p-tyramine, 3-methoxytyramine, N-methyltyramine, 3-indothyronamine, m-octopamine, p-octopamine, and synepherine.

In some embodiments, G¹ in any of the formulae above is selected from:

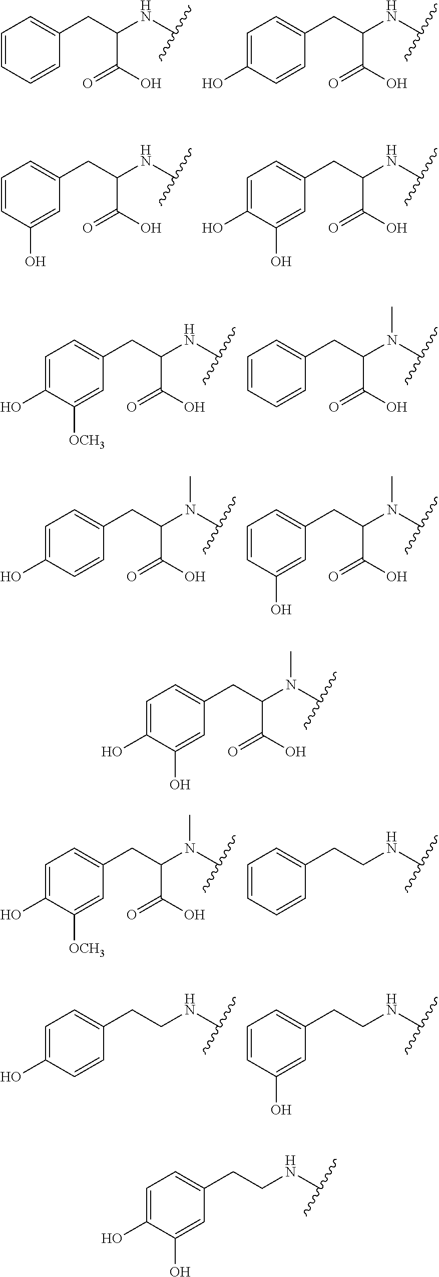

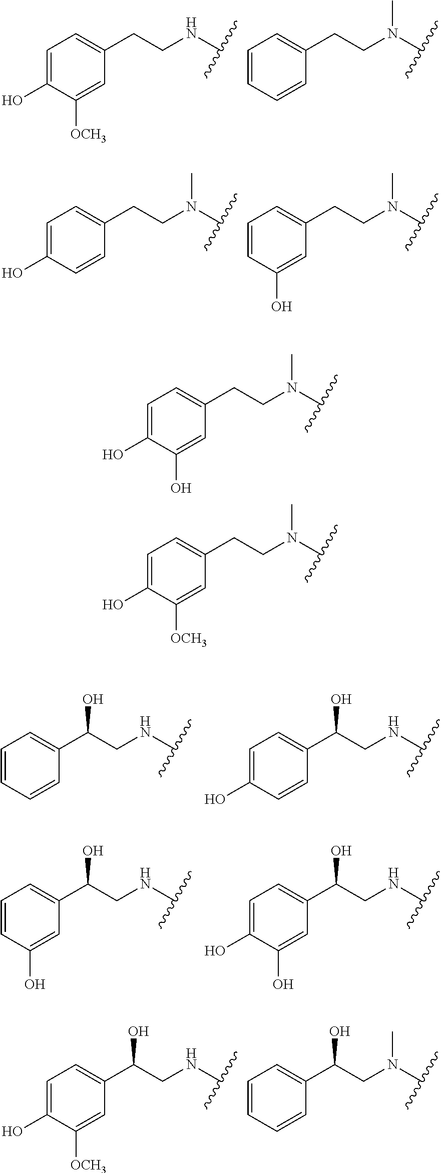

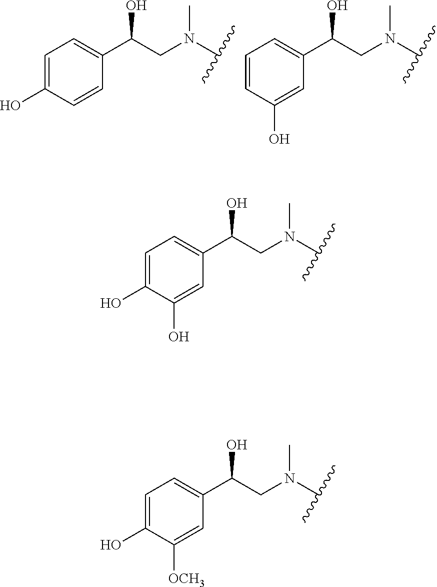

In some embodiments, G¹ in any of the formulae above is selected from:

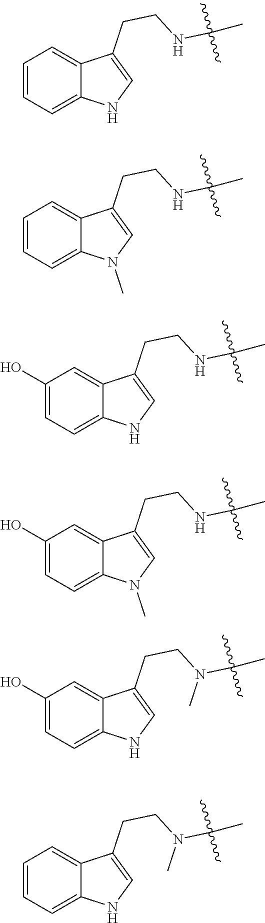

In some embodiments, G² in any of the formulae above is selected from:

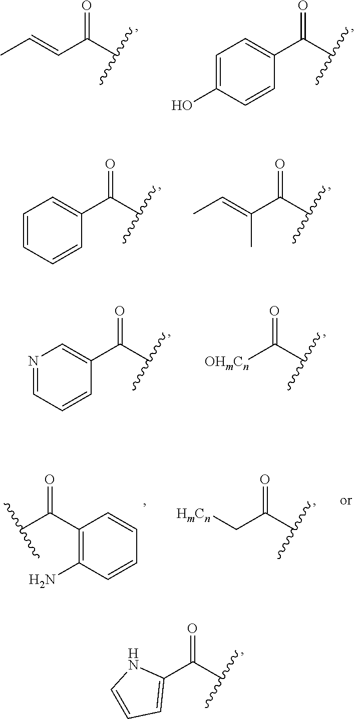

wherein n is 1-35 (e.g., 1-24, 1-18, 1-12, 1-8, or 1-6) and m is an integer dependent upon n to provide a stable saturated, unsaturated, or polyunsaturated aliphatic group.

In some embodiments, G² in any of the formulae above is selected from:

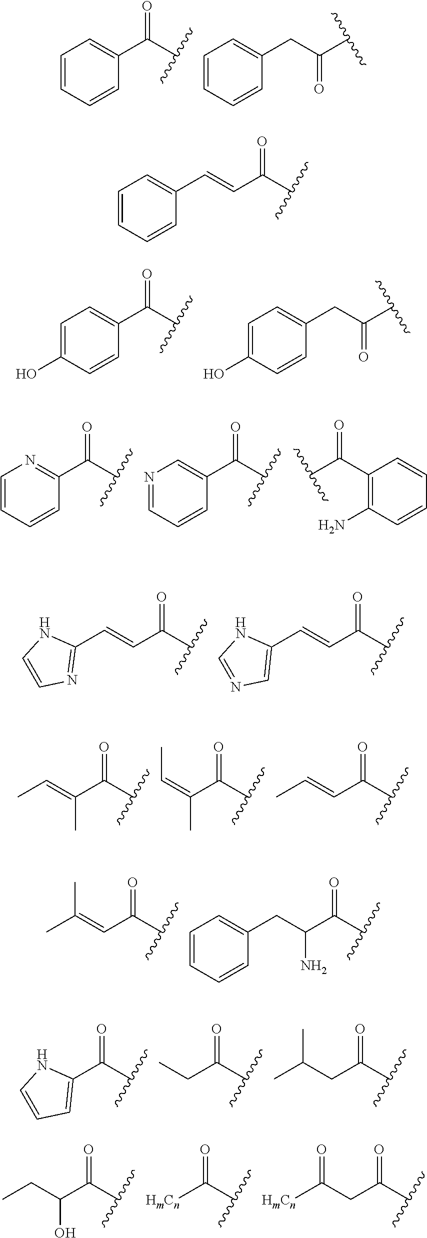

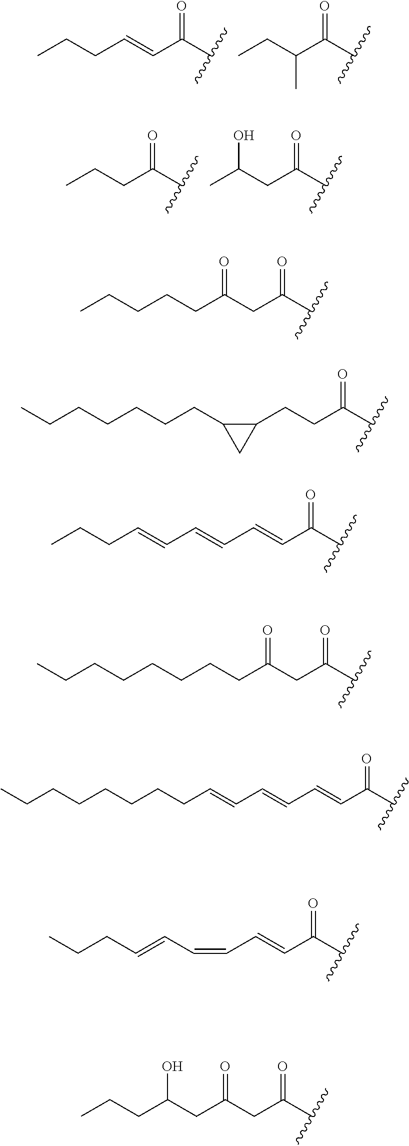

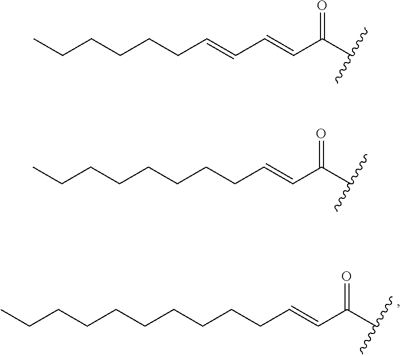

wherein n is 1-35 (e.g., 1-24, 1-18, 1-12, 1-8, or 1-6) and m is an integer dependent upon n to provide a stable saturated, unsaturated, or polyunsaturated aliphatic group.

In some embodiments, G² in any of the formulae above is selected from:

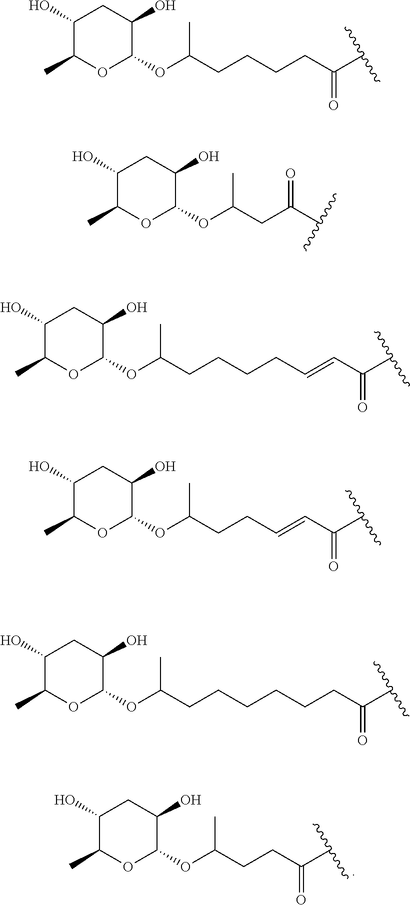 [SMILES: *C(=O)CCCCC(C)O[C@@H]1O[C@@H](C)[C@H](O)C[C@H]1O ; *C(=O)CC(C)O[C@@H]1O[C@@H](C)[C@H](O)C[C@H]1O ; *C(=O)/C=C/CCCCC(C)O[C@@H]1O[C@@H](C)[C@H](O)C[C@H]1O ; *C(=O)/C=C/CCC(C)O[C@@H]1O[C@@H](C)[C@H](O)C[C@H]1O ; *C(=O)CCCCCCC(C)O[C@@H]1O[C@@H](C)[C@H](O)C[C@H]1O ; *C(=O)CCC(C)O[C@@H]1O[C@@H](C)[C@H](O)C[C@H]1O]

In certain embodiments, the MOGL-containing therapeutic compositions described above are characterized in that the substituent at the 3-position of the glucose comprises a phosphate—this may be a simple phosphate (e.g. —OPO₃H₂) or may comprise a di-, tri- or higher phosphate (e.g. —O—(P(O₃H)ₙ—H, where n is an integer greater than 1), or a phosphate derivative such as a salt or an ester. In certain embodiments, the 3-substituent is phosphate. In certain embodiments, the 3-substituent is diphosphate. In certain embodiments, the 3-substituent is triphosphate. In certain embodiments, the composition is provided in a form wherein the phosphate moiety at the 3-position is protonated. In certain embodiments, the compositions is provided in a form wherein the phosphate moiety at the 3-position comprises a salt (e.g. where one or more of —H groups on the phosphate are replaced by a metal cation or organic or inorganic ‘onium’ group).

In certain embodiments, the MOGL-containing therapeutic compositions described above are characterized in that the substituent at the 6-position comprises an optionally substituted moiety selected from the group consisting of: an acyl-linked amino acid, an aromatic acyl group and an aliphatic acyl group. In certain embodiments, the substituent at the 6-position comprises an acyl linked amino acid. In certain embodiments, the amino acid is an alpha amino acid. In certain embodiments, the amino acid comprises a proteinogenic amino acid. In certain embodiments, the amino acid comprises one of the 20 encoded proteogenic amino acids. In certain embodiments, the amino acid is phenylalanine. In certain embodiments, the substituent at the 6-position comprises a peptide linked to the glucose via an ester bond.

In certain embodiments, the substituent at the 6-position comprises an aromatic acyl group. In certain embodiments, the substituent at the 6-position comprises an optionally substituted benzoate. In certain embodiments, the optionally substituted benzoate is selected from the group consisting of: benzoate, anthranilate, and p-hydroxybenzoate. In certain embodiments, the substituent at the 6-position comprises anthranilate.

In certain embodiments, the substituent at the 6-position comprises a heteroaromatic acyl group. In certain embodiments, the substituent at the 6-position comprises an optionally substituted heteroaromatic acyl group with a 6-membered heteroaromatic moiety. In certain embodiments, the substituent at the 6-position comprises a pyridine or pyrimidine carboxylate ester. In certain embodiments, the 6-substituent comprises nicotinate. In certain embodiments, the 6-substituent comprises picolinate. In certain embodiments, the 6-substituent comprises isonicotinate. In certain embodiments, the substituent at the 6-position comprises a heteroaromatic acyl group with a 5-membered heteroaromatic moiety. In certain embodiments, the substituent at the 6-position comprises the ester of a pyrrole or imidazole carboxylic acid. In certain embodiments, the substituent at the 6-position is pyrrole-2-carboxylate.

In certain embodiments, the MOGL-containing therapeutic compositions described above are characterized in that the substituent at the 6-position comprises an optionally substituted aliphatic acyl group. In certain embodiments, the acyl group at the 6-position comprises an optionally substituted aliphatic group. In certain embodiments, the acyl group at the 6-position comprises an optionally substituted C₁₋₃₀ aliphatic group, an optionally substituted C₂₋₂₄ aliphatic group, an optionally substituted C₁₂₋₂₄ aliphatic group, an optionally substituted C₂₋₁₈ aliphatic group, an optionally substituted C₂₋₁₂ aliphatic group, an optionally substituted C₂₋₈ aliphatic group, or an optionally substituted C₁₋₆ aliphatic group. In certain embodiments, the acyl group at the 6-position comprises phenylacetate. In certain embodiments, optionally substituted aliphatic groups are saturated. In certain embodiments, acyl groups at the 6-position have one or more sites of unsaturation. In certain embodiments, the 6-substituent comprises an unsaturated aliphatic group having unsaturation adjacent to the carbonyl of the acyl linkage (e.g. they are alpha-beta unsaturated esters). In certain embodiments, an acyl substituent at the 6-position comprises a C₂₋₈ aliphatic group with alpha beta unsaturation. In certain embodiments, the substituent at the 6-position comprises crotonate. In certain embodiments, the substituent at the 6-position comprises tiglate. In certain embodiments, the substituent at the 6-position comprises angelate. In certain embodiments, the substituent at the 6-position comprises acrylate, methacrylate, or cinnamate. In certain embodiments, the substituent at the 6-position comprises 2-imidazoleacrylate. In certain embodiments, the substituent at the 6-position comprises urocanate.

In some embodiments, G⁶ in any of the formulae above is selected from:

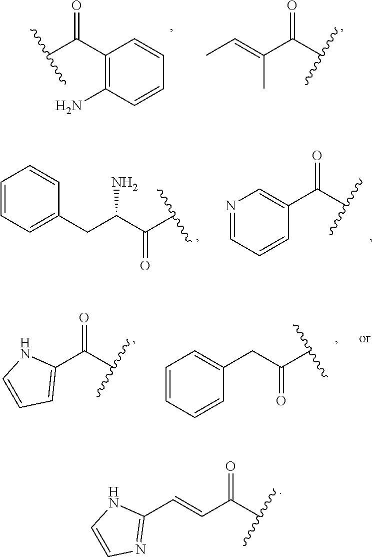

In some embodiments, G⁶ in any of the formulae above is selected from:

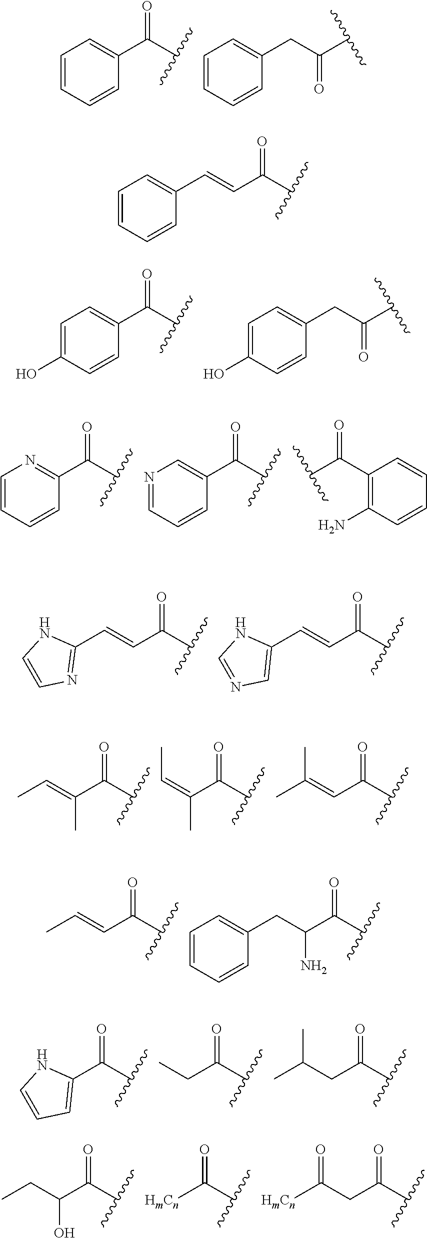

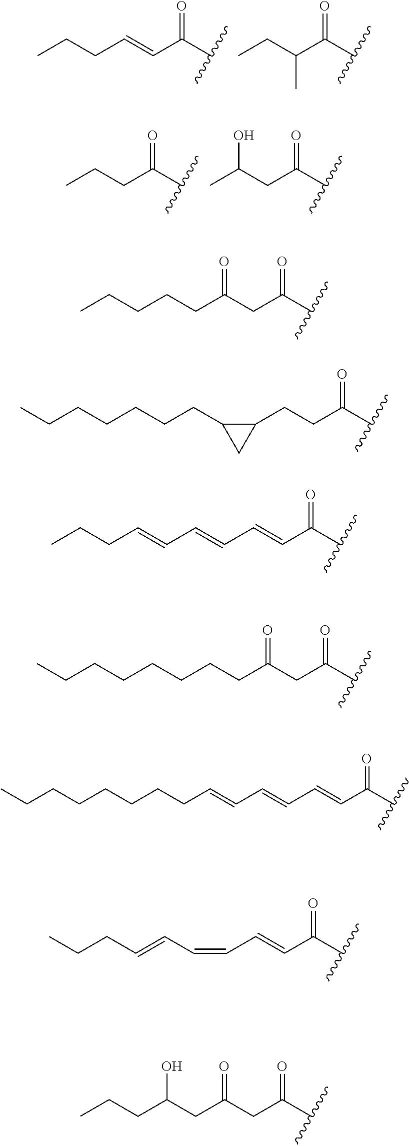

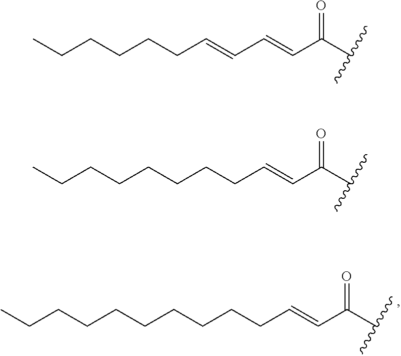

wherein n is 1-35 (e.g., 1-24, 1-18, 1-12, 1-8, or 1-6) and m is an integer dependent upon n to provide a stable saturated, unsaturated, or polyunsaturated aliphatic group.

In some embodiments, G⁶ in any of the formulae above is selected from:

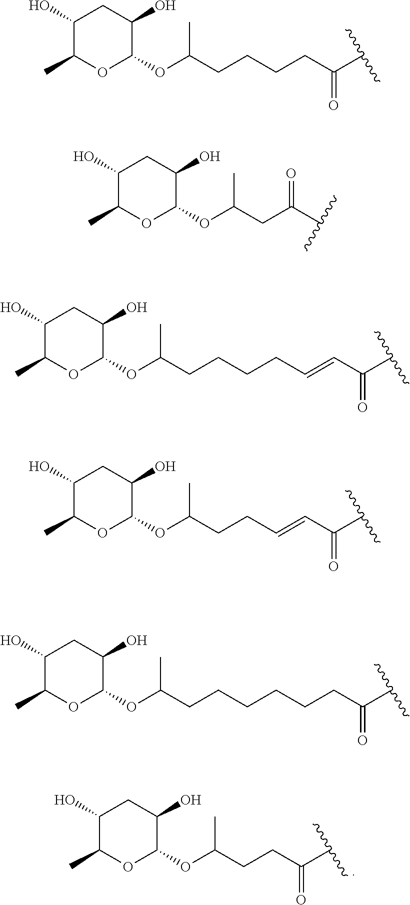 [SMILES: *C(=O)CCCCC(C)O[C@@H]1O[C@@H](C)[C@H](O)C[C@H]1O ; *C(=O)CC(C)O[C@@H]1O[C@@H](C)[C@H](O)C[C@H]1O ; *C(=O)/C=C/CCCCC(C)O[C@@H]1O[C@@H](C)[C@H](O)C[C@H]1O ; *C(=O)/C=C/CCC(C)O[C@@H]1O[C@@H](C)[C@H](O)C[C@H]1O ; *C(=O)CCCCCCC(C)O[C@@H]1O[C@@H](C)[C@H](O)C[C@H]1O ; *C(=O)CCC(C)O[C@@H]1O[C@@H](C)[C@H](O)C[C@H]1O]

In certain embodiments, the present invention encompasses compositions of matter comprising a therapeutically effective amount of one or more compounds of Formula I:

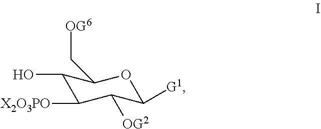

wherein:
G¹ is an optionally substituted moiety selected from the group consisting of: N-linked heterocycle, —OR¹⁰ and —OC(O)R¹¹;
G² is an optionally substituted aliphatic, aromatic, heteroaromatic, or heteroaliphatic acyl group;
X is, independently at each occurrence, selected from the group consisting of —H, an optionally substituted phosphate or polyphosphate moiety, M⁺, and Z⁺; and
G⁶ is an optionally substituted aliphatic, aromatic, heteroaromatic, or heteroaliphatic acyl group,
wherein, R¹⁰ and R¹¹ are each independently selected from the group consisting of: optionally substituted C₁₋₃₂ aliphatic, optionally substituted C₁₋₃₂ heteroaliphatic, optionally substituted aryl, and optionally substituted heteroaryl.

In certain embodiments, the present invention encompasses compositions of matter comprising a therapeutically effective amount of one or more compounds of Formula IV:

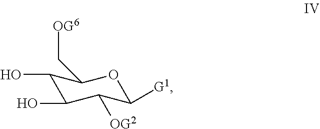

wherein each of G¹, G² and G⁶ is as defined above and in the genera and subgenera herein.

In certain embodiments, the present invention encompasses compositions of matter comprising a therapeutically effective amount of one or more compounds of Formula V:

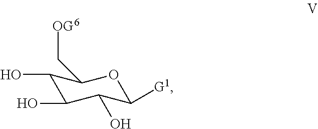

wherein each of G¹ and G⁶ is as defined above and in the genera and subgenera herein.

In certain embodiments, the present invention encompasses compositions of matter comprising a therapeutically effective amount of one or more compounds of Formula VI:

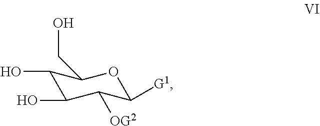

wherein each of G¹ and G² is as defined above and in the genera and subgenera herein.

In certain embodiments, the present invention encompasses compositions of matter comprising a therapeutically effective amount of one or more compounds of Formula VII:

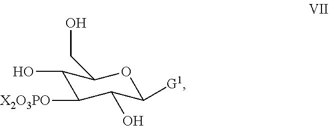

wherein each of G¹ and X is as defined above and in the genera and subgenera herein.

In another aspect, the present invention encompasses methods of improving the health of an animal or of treating or ameliorating a health disorder in an animal by administering to the animal an effective amount of any one or more of the therapeutic compositions described above. In certain embodiments, the method comprises administering such a composition to a mammal. In certain embodiments, the method comprises administering such a composition to a human.

In another aspect, the present invention comprises methods of making therapeutic compositions comprising formulating an effective amount of one or more purified or synthetically-produced MOGLs (or a pharmaceutically-acceptable salt, prodrug or derivative thereof) into a pharmaceutical composition selected from the group consisting of injectible liquid, tablet, capsule, pill, solution or suspension for oral administration, solid for suspension or dissolution into a drinkable or injectible liquid, dermal patch, eye drop, cream, ointment, gel, powder, spray, and inhalable.

In another aspect, the present invention provides pharmaceutical compositions containing MOGLs. In certain embodiments, the invention encompasses a pharmaceutical composition or a single unit dosage form of any of the compounds described above. In certain embodiments, pharmaceutical compositions and single unit dosage forms of the invention comprise a prophylactically or therapeutically effective amount of one or more of the MOGLs describe above, or their pro-drugs, and typically one or more pharmaceutically acceptable carriers or excipients. In a specific embodiment and in this context, the term “pharmaceutically acceptable” means approved by a regulatory agency of the Federal or a state government (or equivalent in other countries) or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Examples of suitable pharmaceutical carriers are described in “Remington's Pharmaceutical Sciences” by E. W. Martin.

Typical pharmaceutical compositions and dosage forms comprise one or more excipients.

Suitable excipients are well-known to those skilled in the art of pharmacy, and non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a patient and the specific active ingredients in the dosage form. The composition or single unit dosage form, if desired, can also contain minor amounts of wetting or emulsifying agents, or pH buffering agents.

Lactose-free compositions of the invention can comprise excipients that are well known in the art and are listed, for example, in the U.S. Pharmocopia (USP) SP (XXI)/NF (XVI). In general, lactose-free compositions comprise an active ingredient, a binder/filler, and a lubricant in pharmaceutically compatible and pharmaceutically acceptable amounts. Preferred lactose-free dosage forms comprise an active ingredient, microcrystalline cellulose, pre-gelatinized starch, and magnesium stearate.

This invention further encompasses anhydrous pharmaceutical compositions and dosage forms comprising active ingredients (e.g any of the MOGLs described above and herein), since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise any one or more MOGLs and one or more compounds that reduce the rate by which an active ingredient will decompose. Such compounds, herein referred to as “stabilizers,” include, but are not limited to, antioxidants such as ascorbic acid, pH buffers, or salt buffers.

The pharmaceutical compositions and single unit dosage forms can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Such compositions and dosage forms will contain a prophylactically or therapeutically effective amount of a prophylactic or therapeutic agent preferably in purified form, together with a suitable amount of carrier so as to provide the form for proper administration to the patient. The formulation should suit the mode of administration. In certain embodiments, the pharmaceutical compositions or single unit dosage forms are sterile and in suitable form for administration to a subject, preferably an animal subject, more preferably a mammalian subject, and most preferably a human subject.

A pharmaceutical composition of the invention is formulated to be compatible with its intended route of administration. Examples of routes of administration include, but are not limited to, parenteral, e.g., intravenous, intradermal, subcutaneous, oral (e.g., inhalation), intranasal, transdermal (topical), transmucosal, intra-tumoral, intra-synovial and rectal administration. In a specific embodiment, the composition is formulated in accordance with routine procedures as a pharmaceutical composition adapted for intravenous, subcutaneous, intramuscular, oral, intranasal or topical administration to human beings. In certain embodiments, a pharmaceutical composition is formulated in accordance with routine procedures for subcutaneous administration to human beings. Typically, compositions for intravenous administration are solutions in sterile isotonic aqueous buffer. Where necessary, the composition may also include a solubilizing agent and a local anesthetic such as lignocane to ease pain at the site of the injection. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form used in the acute treatment of inflammation or a related disorder may contain larger amounts of one or more of the active ingredients it comprises than a dosage form used in the chronic treatment of the same disease. Also, the therapeutically effective dosage form may vary among different types of cancer. Similarly, a parenteral dosage form may contain smaller amounts of one or more of the active ingredients it comprises than an oral dosage form used to treat the same disease or disorder. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration. Typical dosage forms of the invention comprise a compound of the invention, or a pharmaceutically acceptable salt thereof lie within the range of from about 1 mg to about 1000 mg per day, given as a single once-a-day dose in the morning but preferably as divided doses throughout the day taken with food.

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms of the invention are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. A specific binder is a mixture of microcrystalline cellulose and sodium carboxymethyl cellulose sold as AVICEL RC-581. Suitable anhydrous or low moisture excipients or additives include AVICEL-PH-103.TM. and Starch 1500 LM.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

Delayed Release Dosage Forms Active ingredients of the invention can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

### Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention.

### Transdermal, Topical & Mucosal Dosage Forms

Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16th and 18th eds., Mack Publishing, Easton Pa. (1980 & 1990).

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts of the active ingredients can be used to further adjust the properties of the resulting composition.

### II. Chemical Compositions of Matter

In another aspect, the present invention encompasses novel compositions of matter including compositions of novel molecules. While some of the MOGLs are naturally occurring molecules that have been detected in the bodies of nematodes and in some cases have been found in low concentrations in the media in which nematodes are cultured, pure samples of these molecules and in particular bulk samples of the pure MOGLs free from other biological materials are not found in nature. Additionally, many of the MOGLs described above have not been detected in nature, even with the aid of highly sensitive and selective analytical techniques such as HPLC-coupled high resolution mass spectroscopy. As such, many of the compounds described above constitute novel compositions of matter.

In certain embodiments, the present invention provides a pure sample of any of the MOGLs described above and in the genera and subgenera herein. In certain embodiments, the present invention provides samples comprising bulk quantities of such molecules in substantially pure form. In certain embodiments, the present invention provides novel compositions comprising mixtures of between two and ten different MOGLs.

In some embodiments, a provided compound is an isolated compound. In some embodiments, a provided compound is a pure compound (e.g., at least 91%, at least 92%, at least 93%, at least 94%, at least 95%, at least 96%, at least 97%, at least 98%, or at least 99% free of other components).

In some embodiments, a compound or composition described herein is provided outside of a C. elegans worm body. In some embodiments, a compound or composition described herein is provided free of C. elegans tissue or other biological materials typically contained within or excreted by C. elegans.

### III. Therapeutic Methods

In another aspect, the present invention encompasses methods of improving the health of an animal or of treating or ameliorating a health disorder in an animal by administering to the animal an effective amount of any one or more of the therapeutic compositions described above. In certain embodiments, the method comprises administering such a composition to a mammal. In certain embodiments, the method comprises administering such a composition to a human.

### A. MOGLs as Treatments to Improve Mood or Mental State, and to Treat Neurological Disorders

Without being bound by theory or thereby limiting the scope of the present invention, it is believed that MOGLs containing a neurotransmitter-like moiety, for example those derived from serotonin, N-acetyl serotonin, adrenaline, dopamine, tyramine, histidine, or octopamine as well as MOGLs derived from synthetic ligands of neurotransmiltter receptors, e.g. selective serotonin re-uptake inhibitors (SSRIs), have utility as therapeutics to cure or ameliorate neurological disease. Formation of the ester bonds in these neurotransmiltter-derived MOGLs (NeuroMOGs) via esterases (e.g. homologs of the carboxylesterase cest-1.2, such as mammalian cocaine esterase, CES2) is known to be reversible in living systems. Similarly, formation of glycosidic bonds such as those linking glucose to neurotransmitters in the NeuroMOGs is known to be reversible. Moreover, glycosides, including phosphorylated glycosides, are known to be readily transported through the vascular system. Therefore, NeuroMOGs produced in the gut, or NeuroMOG-based therapeutics taken up through the gut, skin, or other modes of administration offer an effective means to alter neurotransmitter-dependent physiological responses by taking advantage of endogenous transport and release mechanisms.

Treatment with NeuroMOGs can be used to improve the mental or emotional state of a patient or to treat anxiety disorders and depression (e.g. by regulating the levels of serotonin or SSRIs), tic disorders (by regulating adrenaline levels), learning disorders and cognitive decline (e.g. in Parkinson patients by elevating dopamine levels), behavioral disorders, and digestive disorders. Selection of the specific moieties attached to the glucose allows for targeting of the NeuroMOGs to specific tissues (e.g. as a result of different lipophilicities) and further enables control of the time scale at which active species (e.g, a neurotransmitters, SSRIs, or neurotransmitter glucoside) are released.

Therefore in certain embodiments, the present invention provides methods of improving the mental or emotional state of an animal (including humans) by administering a therapeutically effective amount of a MOGL comprising a neurotransmitter, or a neurotransmitter-like moiety covalenty linked to the 1-position of the present invention provides methods of treating, ameliorating or curing a neurological or emotional disorder of an animal (including humans) by administering a therapeutically effective amount of a MOGL comprising a neurotransmitter, or a neurotransmitter-like moiety covalenty linked to the 1-position of the MOGL. In certain embodiments, the neurological or emotional disorder comprises anxiety, depression, obsessive or compulsive disorders or behaviors, tics, bipolar disorder, schizophrenia, learning disorders, cognitive decline, behavioral disorders, learning disability, hyperactivity and the like.

In certain embodiments, such methods comprise administering an effective dose of a MOGL having a neurotransmitter (or a derivative or precursor of a neurotransmitter) linked to the 1-position of the glucose. In certain embodiments, such compositions comprise one or more compounds selected from the group:

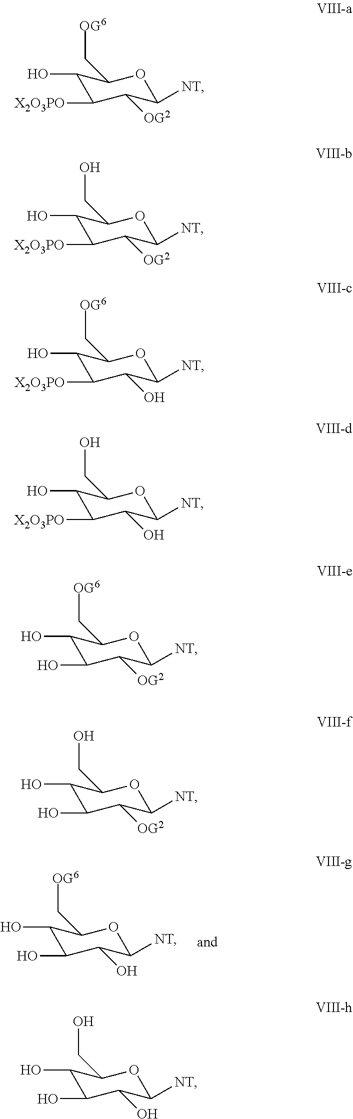

where each of G2, G3, G6, X, and —NT is as defined above and in the genera and subgenera herein.

### B. MOGLs as Kinase Modulators

Kinases play a central role for many types of human diseases. For example, (Cicenas J. Zalyte E, Bairoch A, Gaudet P. Kinases and Cancer. Cancers (Basel). 2018; 10(3):63. Published 2018 Mar. 1. doi:10.3390/cancers10030063) report that mutated kinases that are constitutively active are drivers of many types of cancers, e.g. the V600E mutation of BRAF colorectal cancer, melanoma, thyroid cancer, and non-small cell lung cancer. Other examples include driver mutations in KIT, EGFR, and FTL3. In addition to mutations, epigenetic changes can result in cancer-driving changes of kinase expression levels. As a result, kinase inhibitors and modulators have been a major focus of cancer research over the past 40 years, which has yielded important cancer drugs in current clinical use, e.g. imatinib (Gleevec), which can extend survival of chronic myelogenous leukemia patients often by a decade or more. Most kinases bind ATP or other nucleotides, and many synthetic kinase inhibitors act as ATP-competitive mechanism or otherwise interact with the nucleotide-binding domain, whereby additional interactions with nearby hydrophobic pockets often play an important role. See for example, Roskoski, Pharmacol. Res., 100:1-23 (2015).

Without being bound by theory, or thereby limiting the scope of the present invention, it is believed the MOGLs described above that comprise a nucleobase or other aromatic moiety, e.g. indole, 5-hydroxyindole, anthranilic acid, or nicotinic acid at the 1-position can play a role in regulating kinase activity and therefore have utility for the treatment of cancer and other kinase dependent disorders or diseases including, for example, hypertension, Parkinson's disease, and autoimmune diseases.

Members of this family of nucleotide-related MOGLs (NuMOGs), representative members of which were recently discovered in the model organism C. elegans, structurally mimic ATP and other nucleotides that kinases are known to bind to, and feature additional hydrophilic and hydrophobic moieties. The combination of polar (phosphate sugar) and less polar (acyl moieties) moieties in the structures of the NuMOGs can be used to tailor affinity and specificity to different kinases, which can be used to target disease-relevant kinases selectively. A subset of NuMOGs featuring one or two acyl groups may also serve as a precursor or pro-drugs of NuMOGs with fewer acyl groups, based on the finding that enzymes of the carboxylesterase family (e.g. CES2 in humans, a homolog of cest-1.2 in C. elegans) are able to hydrolyze ester bonds. The ability to tailor lipophilicity via additional acyl moieties facilitates design of NuMOGs or pro-drugs of NuMOGs that have desirable properties, such as high bioavalability in the gut or high tissue penetration. As inhibitors and modulators of kinase activity, NUMOGs can be used to treat cancer, but also offer new opportunities for the treatment of other diseases in which kinases are known to play an important role, including hypertension, Parkinson's disease, and autoimmune diseases. See for example, Roskoski, Pharmacol. Res., 100:1-23 (2015).

Therefore, in certain embodiments, the present invention provides methods of amelieorating or curing a kinase-dependent disease or disorder. In certain embodiments, such methods comprise administering to a patient a pharmaceutically effective dose of one or more MOGLs. In certain embodiments, the MOGL(s) administered are characterized in that they comprise a nucleobase or other aromatic moiety at the 1-position. In certain embodiments, such MOGLs are selected from the group consisting of:

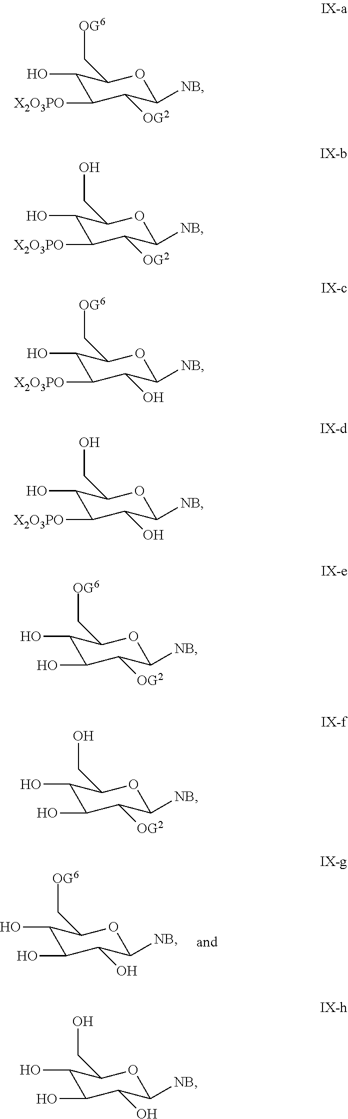

where each of G2, G3, G6, —NB, and X is as defined above and in the genera and subgenera herein.

### C. MOGLs as Therapies for Modulation of Nucleoside Metabolism

Upregulation of nucleoside metabolism is a hallmark of cancer, and correspondingly chemotherapeutics that target nucleoside biosynthesis and oligonucleotide production are important components of cancer treatments. Similar to cancerous cells, virally infected cells also increase nucleotide synthesis, for example by inhibiting the tumor suppressor p53, and enhanced nucleotide production is necessary for viral replication. Correspondingly, nucleotide metabolism is an important target of established treatments of cancer and viral diseases. See for example, Ariav et. al., Science Advances, 7(21):1-8 (May 19, 2021). In certain embodiments, the present invention relates to therapies for the treatment of disorders that result in or arise from changes to nucleotide synthesis including, but not limited to cancer and viral diseases.

In certain embodiments, such methods comprise treating an animal with a therapeutically effective amount of a MOGLs comprising a nucleoside or nucleoside derivative (e.g., adenine glucoside, 4-N-methylcytosine glucoside, guanosine, methylguanosine, or methyladenine).

Without being bound by theory or thereby limiting the scope of this invention, it is believed members of this family of nucleotide-related MOGLs (NuMOGs), structurally mimic canonical ribonucleotides and can interfere with production of ribonucleotides by inhibiting enzymes required for their biosynthesis. In addition, NuMOGs, due to their structural similarity with ribonucleotides, can interfere with assembly of oligonucleotides, e.g. RNA and DNA and thereby interfere with cell division (e.g. of tumor cells) or viral replication. These properties indicate that NuMOGs can be useful as anti-cancer drugs and antivirals. A subset of NuMOGs featuring one or two acyl groups may also serve as a precursor or pro-drugs of NuMOGs with fewer acyl groups, based on the finding that enzymes of the carboxylesterase family (e.g. CES2 in humans, a homolog of cest-1.2 in C. elegans) are able to hydrolyze ester bonds. The ability to tailor lipophilicity via additional acyl moieties facilitates design of NuMOGs or pro-drugs of NuMOGs that have desirable properties, such as high bioavailability in the gut or high tissue penetration.

Therefore, in certain embodiments, the present invention provides methods of amelieorating or curing a nucleotide synthesis-related disease or disorder. In certain embodiments, such methods comprise administering to a patient a pharmaceutically effective dose of one or more MOGLs. In certain embodiments, the MOGL(s) administered are characterized in that they comprise a a nucleobase or other aromatic moiety at the 1-position. In certain embodiments, such MOGLs are selected from the group consisting of:

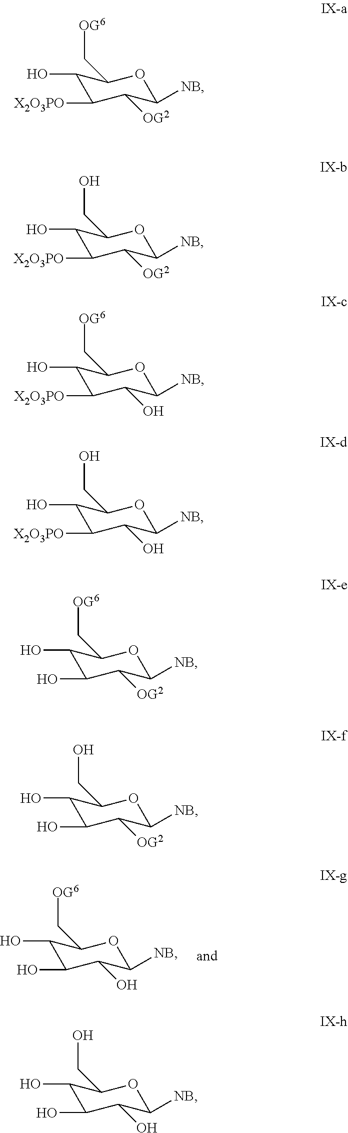

where each of G2, G3, G6, NB, and X is as defined above and in the genera and subgenera herein.

### D. MOGLs as Therapies to Regulate Nutrient Responses and Growth

Modular glucosides (MOGLs) derived from glucosides of methylcrotonate-related moieties (MeMOGs), which are naturally produced in a TOR- (Target Of Rapamycin-) dependent manner in some organisms (e.g. the model organism C. elegans) offer new opportunities for the treatment of important human disease. The TOR signaling network, see for example, Loewith and Hall, Genetics, 189(4):1177-1201 (2011), is a central regulator of nutrient-dependent signaling and growth, and the amino acid leucine and its downstream metabolite 3-methylcrotonate are known to play an important role in regulating TOR function. Our finding that MeMOG production is dependent on TOR indicates that MeMOGs offer new perspectives for modulating TOR. Modulating TOR activity, e.g. via the FDA-approved drug rapamycin, has been employed successfully in three major therapeutic areas: immunosuppression/organ transplantation, cancer, and coronary artery disease. Similarly, MeMOGs can be used (i) to suppress or otherwise modulate immune responses (e.g. in the context of organ rejection or autoimmune disorder), (ii) to suppress proliferation of tumor cells (in analogy to the action of rapamycin, which blocks cancer growth directly and further prevents the growth of new blood vessels (angiogenesis) that supply oxygen and nutrients to tumors), and (iii) prevent restenosis after angioplasty (again in analogy to rapamycin).

Therefore, in certain embodiments, the present invention provides methods of amelieorating or curing a disease or disorder responsive to regulation of TOR function. In certain embodiments, such methods comprise administering to a patient a pharmaceutically effective dose of one or more MOGLs. In certain embodiments, the MOGL(s) administered are characterized in that they comprise an alpha-beta unsaturated acyl moiety. In certain embodiments, such MOGLs comprise a crotonate or methyl crotonate moiety. In certain embodiments, such MOGLs are characterized in that a substituent at the 1-position independently comprises a C₃₋₈ acyl group with alpha beta unsaturation. In certain embodiments, the substituent at the 1-position comprises crotonate. In certain embodiments, the substituent at the 1-position comprises methylcrotonate. In certain embodiments, the substituent at the 1-position comprises tiglate. In certain embodiments, the substituent at the 1-position comprises angelate. In certain embodiments, the subsituent at the 1-position comprises acrylate, methacrylate, 3-methylcrotonate, or isocrotonate.

In certain embodiments, the present invention provides methods of amelieorating or curing a disease or disorder responsive to regulation of TOR function comprising administering to a patient a therapeutically effective dose of one or more compounds selected from the group:

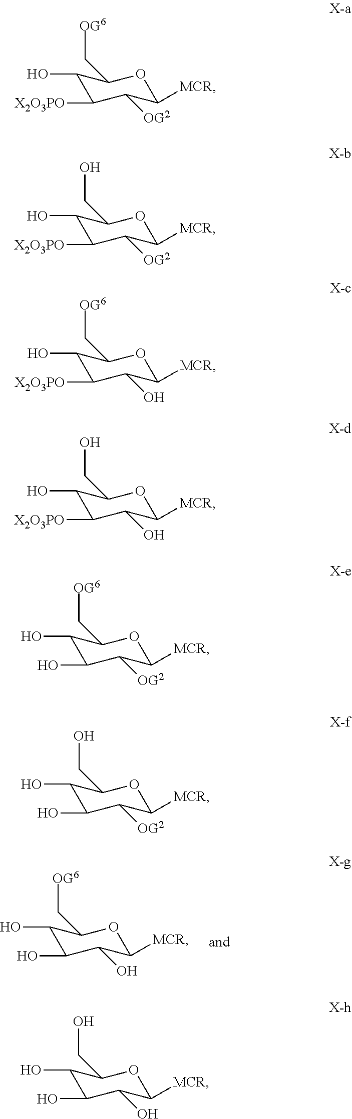

where each of G², G³, G⁶, X, and -MCR is as defined above and in the genera and subgenera herein, and

In certain embodiments, the method comprises treatment with an effective amount of a MeMOG based on alpha or beta-glycosides of 3-methylcrotonate, isobutyric acid, or isovaleric acid, optionally bearing a phosphate or phosphate derivative in position 3 of the sugar, as well as acyl groups selected from any variable substituent as defined above for G² or G⁶ (i.e., at the oxygens in positions 2 and 6 of the glucose). Selection of the specific moieties attached to the glucose allows for targeting of the MeMOGs to specific tissues (e.g. as a result of different lipophilicities) and further enables control of the time scale at which active species (e.g, a monoacylated 3-methylcrotonyl glucoside) are released.

### E. MOGLs as Proteasome Modulators

Without being bound by theory or thereby limiting the scope of the present invention, it is believed that MOGLs containing a neurotransmitter-like moiety (e.g., NeuroMOGs) have utility as modulators of the proteasome. Function of the proteasome (i.e., protein degradation) requires assembly of seven well-folded subunits to form a ring complex, and conformational changes in one or more of the subunits can be expected to significantly enhance or reduce activity of proteolysis. Resulting modulation of proteasome activity can provide important advantages for the treatment of human disease. Inhibition of proteasome function is one important strategy for the treatment of cancer. See for example, Irvine et. al., J Cell Commun. Signal, 5(2): 101-110 (2011); Rastogi and Mishra, Cell Div., 7:26, 1-10 (2012); Adams, Cancer Cell, 5(5): 417-421 (2004). Specific structural MOGLs could also increase proteasome function, which would offer new treatment opportunities for many aging-related diseases and neurodegenerative disorders that are derived from protein misfolding, including Alzheimer's and Parkinson's and Huntington's disease. See for example, Hodgson et. al. Translational Neurodegeneration 6:6, 1-13 (2017).

In certain embodiments, the present invention provides methods of treating a disease or disorder responsive to modulation of the proteasome, comprising administering to a patient in need thereof a therapeutically effective amount of a compound (e.g., MOGL) described herein. In some embodiments, a MOGL is a proteasome inhibitor. In some embodiments, a MOGL is a proteasome activator. In some embodiments, a compound is a MOGL having a neurotransmitter (or a derivative or precursor of a neurotransmitter) linked to the 1-position of the glucose. In certain embodiments, a compound is selected from:

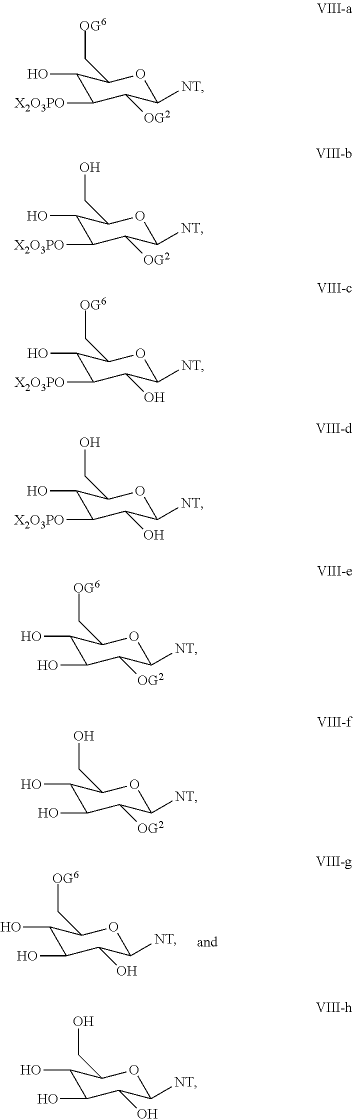

where each of G2, G3, G6, X, and —NT is as defined above and in the genera and subgenera herein.

The following numbered embodiments, while non-limiting, are exemplary of certain aspects of the disclosure:

1. A glucose derivative substituted at the 1-, 2-, 3-, and 6-positions, wherein:
the substituent at the 1-position is selected from the group consisting of a nucleobase, an N-linked heterocycle, an acyl- or ether-linked aromatic moiety, an optionally unsaturated acyl group, an ether-linked aromatic moiety substituted with an amine or aminoalkyl group;
the substituent at the 2-position comprises an optionally substituted aromatic, heteroaromatic, or aliphatic acyl group;
the substituent at the 3-position comprises a phosphate, diphosphate, triphosphate, a sulfate, or a derivative of any of these; and
the substituent at the 6-position comprises an acyl-linked amino acid, or an optionally substituted aromatic or aliphatic acyl group.

2. The glucose derivative of embodiment 1, characterized in that the glucose derivative is not substituted at the 4-position.

3. A glucose derivative substituted at the 1-, 2-, and 3-positions, wherein:
the substituent at the 1-position is selected from the group consisting of a nucleobase, an N-linked heterocycle, an acyl- or ether-linked aromatic moiety, an optionally unsaturated acyl group, an ether-linked aromatic moiety substituted with an amine or aminoalkyl group;
the substituent at the 2-position comprises an optionally substituted aromatic, heteroaromatic, or aliphatic acyl group; and
the substituent at the 3-position comprises a phosphate, diphosphate, triphosphate, a sulfate, or a derivative of any of these.

4. The glucose derivative of embodiment 3, characterized in that the glucose derivative is not substituted at the 4- or 6-position.

5. A glucose derivative substituted at the 1-, 3-, and 6-positions, wherein:
the substituent at the 1-position is selected from the group consisting of a nucleobase, an N-linked heterocycle, an acyl- or ether-linked aromatic moiety, an optionally unsaturated acyl group, an ether-linked aromatic moiety substituted with an amine or aminoalkyl group;
the substituent at the 3-position comprises a phosphate, diphosphate, triphosphate, a sulfate, or a derivative of any of these; and
the substituent at the 6-position comprises an acyl-linked amino acid, or an optionally substituted aromatic or aliphatic acyl group.

6. The glucose derivative of embodiment 5, characterized in that the glucose derivative is not substituted at the 2- or 4-position.

7. A glucose derivative substituted at the 1- and 3-positions, wherein:
the substituent at the 1-position is selected from the group consisting of a nucleobase, an N-linked heterocycle, an acyl- or ether-linked aromatic moiety, an optionally unsaturated acyl group, an ether-linked aromatic moiety substituted with an amine or aminoalkyl group; and
the substituent at the 3-position comprises a phosphate, diphosphate, triphosphate, a sulfate, or a derivative of any of these.

8. The glucose derivative of embodiment 7, characterized in that the glucose derivative is not substituted at the 2-, 4-, or 6-position.

9. A glucose derivative substituted at the 1-, 2-, and 6-positions, wherein:
the substituent at the 1-position is selected from the group consisting of a nucleobase, an N-linked heterocycle, an acyl- or ether-linked aromatic moiety, an optionally unsaturated acyl group, an ether-linked aromatic moiety substituted with an amine or aminoalkyl group;
the substituent at the 2-position comprises an optionally substituted aromatic, heteroaromatic, or aliphatic acyl group; and
the substituent at the 6-position comprises an acyl-linked amino acid, or an optionally substituted aromatic or aliphatic acyl group.

10. The glucose derivative of embodiment 9, characterized in that the glucose derivative is not substituted at the 3- or 4-position.

11. A glucose derivative substituted at the 1- and 2-, positions, wherein:
the substituent at the 1-position is selected from the group consisting of a nucleobase, an N-linked heterocycle, an acyl- or ether-linked aromatic moiety, an optionally unsaturated acyl group, an ether-linked aromatic moiety substituted with an amine or aminoalkyl group; and
the substituent at the 2-position comprises an optionally substituted aromatic, heteroaromatic, or aliphatic acyl group.

12. The glucose derivative of embodiment 11, characterized in that the glucose derivative is not substituted at the 3-, 4-, or 6-position.

13. A glucose derivative substituted at the 1- and 6-positions, wherein:
the substituent at the 1-position is selected from the group consisting of a nucleobase, an N-linked heterocycle, an acyl- or ether-linked aromatic moiety, an optionally unsaturated acyl group, an ether-linked aromatic moiety substituted with an amine or aminoalkyl group; and
the substituent at the 6-position comprises an acyl-linked amino acid, or an optionally substituted aromatic or aliphatic acyl group.

14. The glucose derivative of embodiment 13, characterized in that the glucose derivative is not substituted at the 2-, 3-, or 4-position.

15. The glucose derivative of any one of the preceding embodiments, wherein the N-linked heterocycle is a nucleobase (e.g., adenine, cytosine, guanine, thymine, or uracil).

16. The glucose derivative of any one of the preceding embodiments, wherein the substituent at the 1-position comprises an acyl- or ether-linked aromatic moiety substituted with an amine or an aminoalkyl group.

17. The glucose derivative of any one of the preceding embodiments, wherein the substituent at the 1-position comprises a phenolic ether where the phenyl ring of the phenol is substituted with an aminoalkyl group.

18. The glucose derivative of any one of the preceding embodiments, wherein the substituent at the 2-position comprises an optionally substituted benzoate.

19. The glucose derivative of any one of the preceding embodiments, wherein the substituent at the 2-position comprises the ester of a pyrrole or imidazole carboxylic acid.

20. The glucose derivative of any one of the preceding embodiments, wherein the substituent at the 2-position comprises an optionally substituted C₁₋₆ aliphatic group.

21. The glucose derivative of any one of the preceding embodiments, wherein the substituent at the 2-position comprises acrylate, methacrylate, 3-methylcrotonate, isocrotonate, or optionally substituted cinnamate.

22. The glucose derivative of any one of the preceding embodiments, wherein the substituent at the 3-position of the glucose comprises a phosphate (e.g. —OPO₃H₂).

23. The glucose derivative of any one of the preceding embodiments, wherein the substituent at the 6-position comprises an acyl linked amino acid (e.g., an alpha amino acid).

24. The glucose derivative of any one of the preceding embodiments, wherein the substituent at the 6-position comprises an optionally substituted aliphatic acyl group.

25. The glucose derivative of any one of the preceding embodiments, wherein an N-linked heterocyclic group is heteroaryl.

26. The glucose derivative of any one of the preceding embodiments, wherein each aromatic group is independently aryl.

27. A compound that is any one of the glucose derivatives of embodiments 1-26.

28. A compound of Formula I:

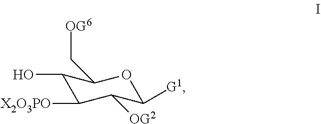

or a pharmaceutically acceptable salt thereof,
wherein:
G¹ is an optionally substituted moiety selected from the group consisting of: N-linked heterocycle, —OR¹⁰ and —OC(O)R¹¹;
G² is an optionally substituted aliphatic, aromatic, heteroaromatic, or heteroaliphatic acyl group;
X is, independently at each occurrence, selected from the group consisting of —H, an optionally substituted phosphate or polyphosphate moiety, M⁺, and Z⁺;
G⁶ is an optionally substituted aliphatic, aromatic, heteroaromatic, or heteroaliphatic acyl group,
R¹⁰ and R¹¹ are each independently selected from the group consisting of: optionally substituted C₁₋₃₂ aliphatic, optionally substituted C₁₋₃₂ heteroaliphatic, optionally substituted aryl, and optionally substituted heteroaryl;
M⁺ is any metal cation; and
Z⁺ is an organic or inorganic ‘onium’ group comprising at least one nitrogen-, phosphorous-, or sulfur-based cation.

29. A compound of Formula I:

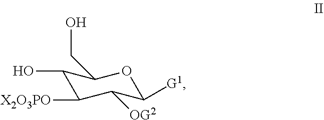

or a pharmaceutically acceptable salt thereof,
wherein:
G¹ is an optionally substituted moiety selected from the group consisting of: N-linked heterocycle, —OR¹⁰ and —OC(O)R¹¹;
G² is an optionally substituted aliphatic, aromatic, heteroaromatic, or heteroaliphatic acyl group;
R¹⁰ and R¹¹ are each independently selected from the group consisting of: optionally substituted C₁₋₃₂ aliphatic, optionally substituted C₁₋₃₂ heteroaliphatic, optionally substituted aryl, and optionally substituted heteroaryl;
X is, independently at each occurrence, selected from the group consisting of —H, an optionally substituted phosphate or polyphosphate moiety, M⁺, and Z⁺;
M⁺ is any metal cation; and
Z⁺ is an organic or inorganic ‘onium’ group comprising at least one nitrogen-, phosphorous-, or sulfur-based cation.

30. A compound of Formula III:

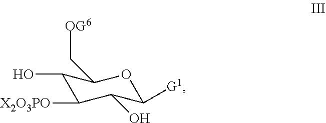

or a pharmaceutically acceptable salt thereof,
wherein:
G¹ is an optionally substituted moiety selected from the group consisting of: N-linked heterocycle, —OR¹⁰ and —OC(O)R¹¹;
X is, independently at each occurrence, selected from the group consisting of —H, an optionally substituted phosphate or polyphosphate moiety, M⁺, and Z⁺;
G⁶ is an optionally substituted aliphatic, aromatic, heteroaromatic, or heteroaliphatic acyl group; where, R¹⁰ and R¹¹ are each independently selected from the group consisting of: optionally substituted C₁₋₃₂ aliphatic, optionally substituted C₁₋₃₂ heteroaliphatic, optionally substituted aryl, and optionally substituted heteroaryl;
M⁺ is any metal cation; and
Z⁺ is an organic or inorganic ‘onium’ group comprising at least one nitrogen-, phosphorous-, or sulfur-based cation.

31. A compound of Formula IV:

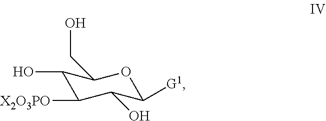

or a pharmaceutically acceptable salt thereof,
wherein:
G¹ is an optionally substituted moiety selected from the group consisting of: N-linked heterocycle, —OR¹⁰ and —OC(O)R¹¹;
X is, independently at each occurrence, selected from the group consisting of —H, an optionally substituted phosphate or polyphosphate moiety, M⁺, and Z⁺;
R¹⁰ and R¹¹ are each independently selected from the group consisting of optionally substituted C₁₋₃₂ aliphatic, optionally substituted C₁₋₃₂ heteroaliphatic, optionally substituted aryl, and optionally substituted heteroaryl;
M⁺ is any metal cation; and
Z⁺ is an organic or inorganic ‘onium’ group comprising at least one nitrogen-, phosphorous-, or sulfur-based cation.

32. A compound of Formula V:

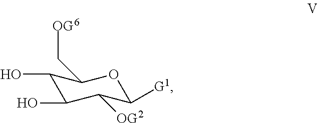

or a pharmaceutically acceptable salt thereof,
G¹ is an optionally substituted moiety selected from the group consisting of: N-linked heterocycle, —OR¹⁰ and —OC(O)R¹¹;
G² is an optionally substituted aliphatic, aromatic, heteroaromatic, or heteroaliphatic acyl group;
G⁶ is an optionally substituted aliphatic, aromatic, heteroaromatic, or heteroaliphatic acyl group; and
R¹⁰ and R¹¹ are each independently selected from the group consisting of optionally substituted C₁₋₃₂ aliphatic, optionally substituted C₁₋₃₂ heteroaliphatic, optionally substituted aryl, and optionally substituted heteroaryl.

33. A compound of Formula VI:

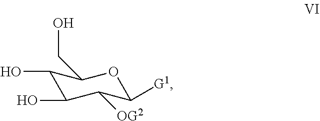

or a pharmaceutically acceptable salt thereof,
G¹ is an optionally substituted moiety selected from the group consisting of: N-linked heterocycle, —OR¹⁰ and —OC(O)R¹¹;
G² is an optionally substituted aliphatic, aromatic, heteroaromatic, or heteroaliphatic acyl group; and
R¹⁰ and R¹¹ are each independently selected from the group consisting of optionally substituted C₁₋₃₂ aliphatic, optionally substituted C₁₋₃₂ heteroaliphatic, optionally substituted aryl, and optionally substituted heteroaryl.

34. A compound of Formula VII:

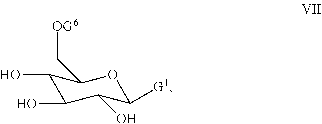

or a pharmaceutically acceptable salt thereof,
G¹ is an optionally substituted moiety selected from the group consisting of: N-linked heterocycle, —OR¹⁰ and —OC(O)R¹;
G⁶ is an optionally substituted aliphatic, aromatic, heteroaromatic, or heteroaliphatic acyl group; and
R¹⁰ and R¹¹ are each independently selected from the group consisting of optionally substituted C₁₋₃₂ aliphatic, optionally substituted C₁₋₃₂ heteroaliphatic, optionally substituted aryl, and optionally substituted heteroaryl.

35. A compound having the formulae:

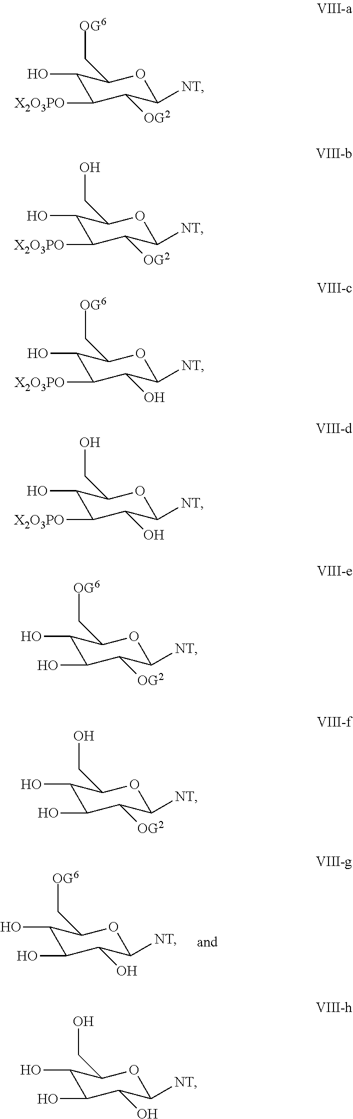

wherein G¹, G², and X are as defined in the preceding embodiments and
—NT comprises a neurotransmitter, or a derivative or precursor of a neurotransmitter linked to the glucose through any suitable atom.

36. The compound of embodiment 35, wherein the moiety —NT comprises a monoamine neurotransmitter or a derivative or precursor thereof.

37. The compound of embodiment 35, wherein —NT comprises a catecholamine neurotransmitter or a derivative or precursor thereof.

38. The compound of embodiment 35, wherein —NT is selected from the group consisting of: dopamine, norepinepherine, epinepherine, histamine, and serotonin.

39. The compound of embodiment 35, wherein —NT is selected from tryptamine, phenethylamine, N-methylphenethylamine, phenethanolamine, m-tyramine, p-tyramine, 3-methoxytyramine, N-methyltyramine, 3-indothyronamine, m-octopamine, p-octopamine, and synepherine.

40. The compound of embodiment 35, wherein —NT is selected from:

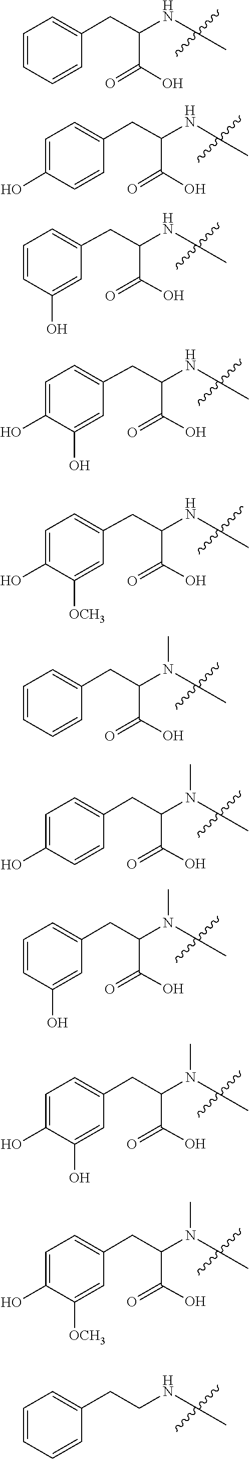

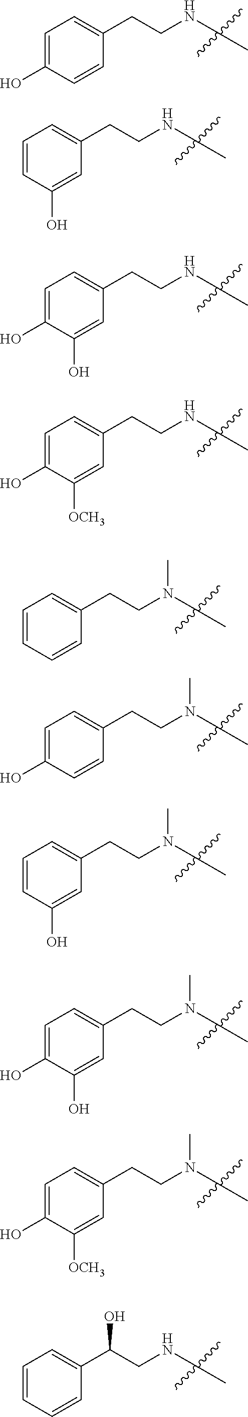

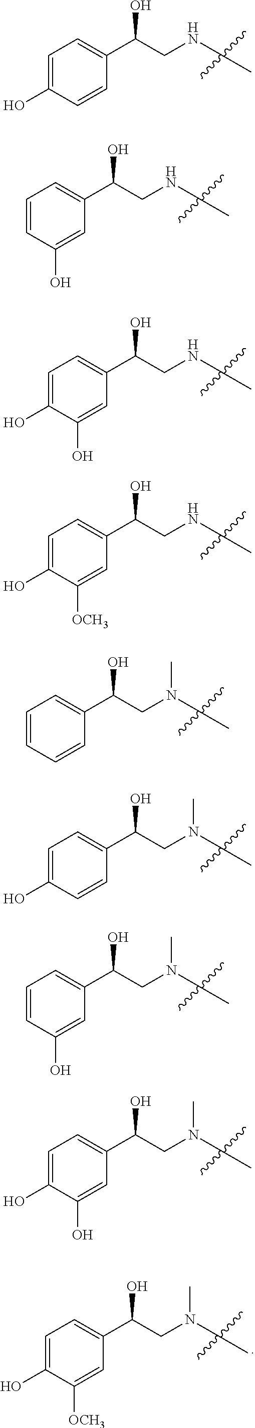

41. The compound of embodiment 35, wherein —NT is selected from:

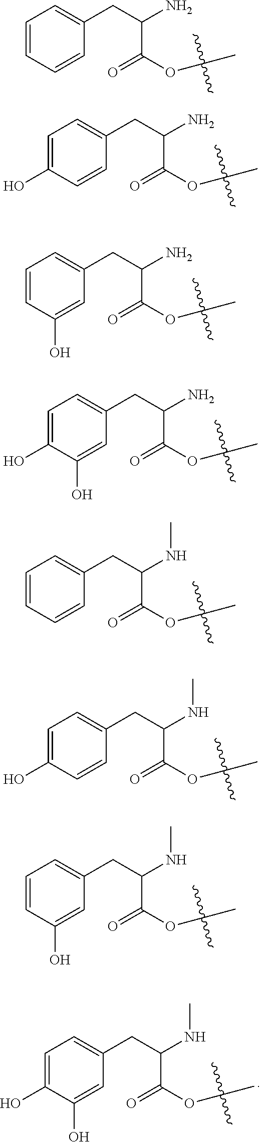

42. The compound of embodiment 35, wherein —NT is selected from:

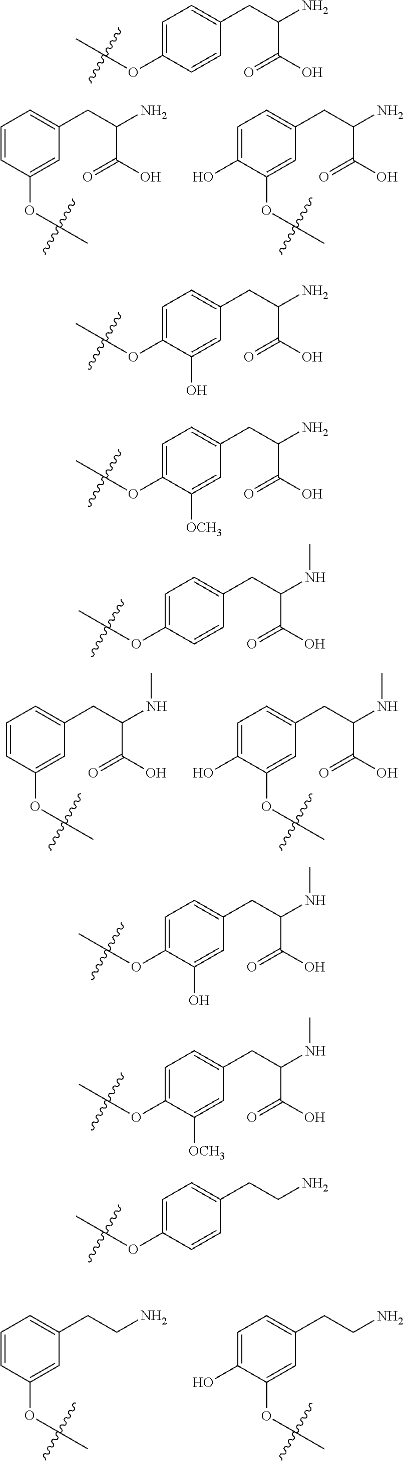

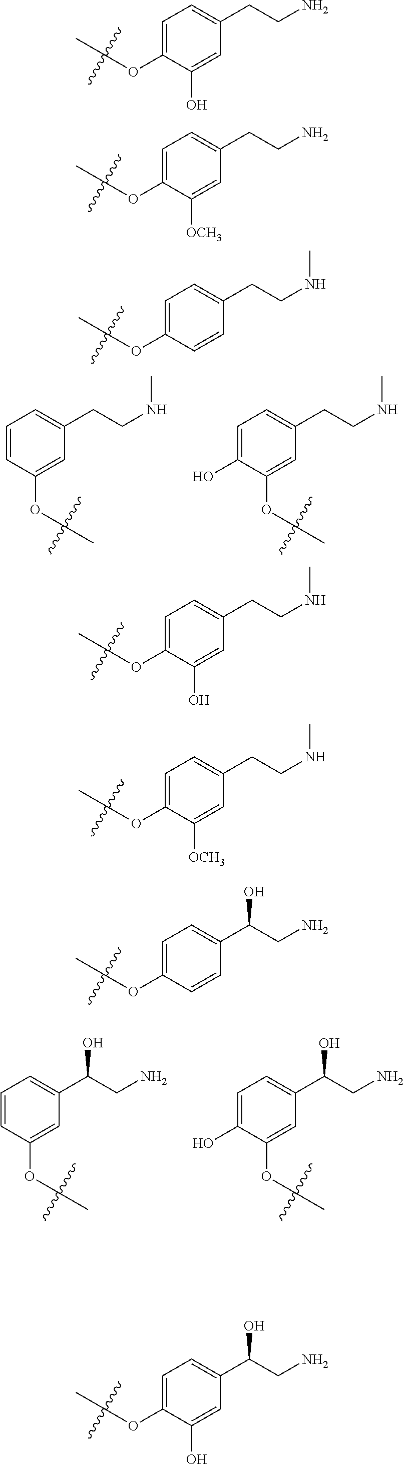

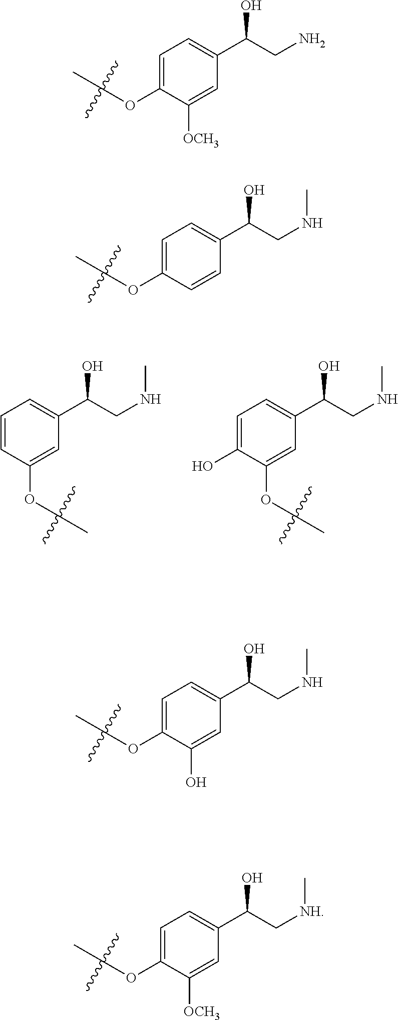 [SMILES: *Oc1ccc([C@@H](O)CN)cc1OC ; *Oc1ccc([C@@H](O)CNC)cc1 ; *Oc1cccc([C@@H](O)CNC)c1 ; *Oc1cc([C@@H](O)CNC)ccc1O ; *Oc1ccc([C@@H](O)CNC)cc1O ; *Oc1ccc([C@@H](O)CNC)cc1OC]

43. The compound of embodiment 35, wherein —NT is selected from:

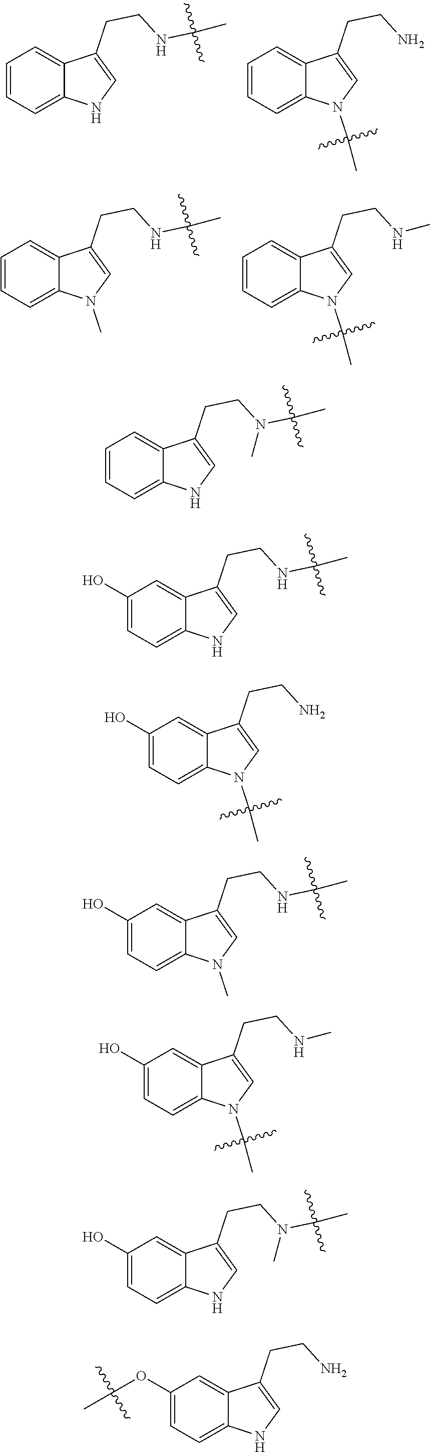

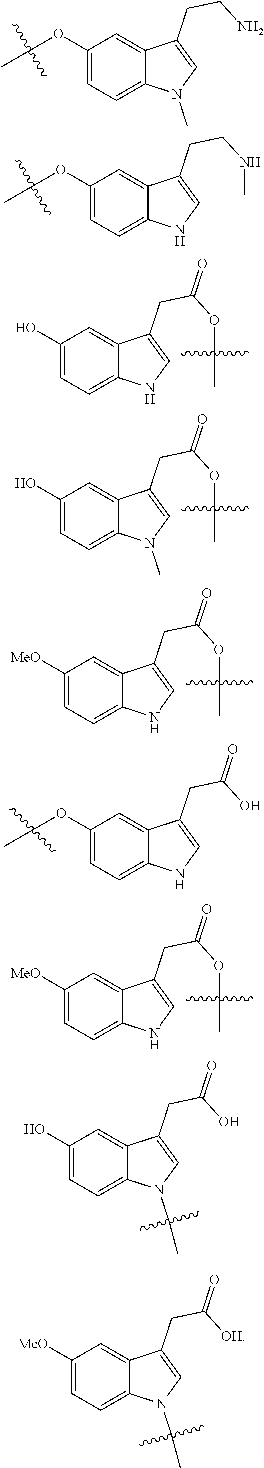

44. The compound having the formulae:

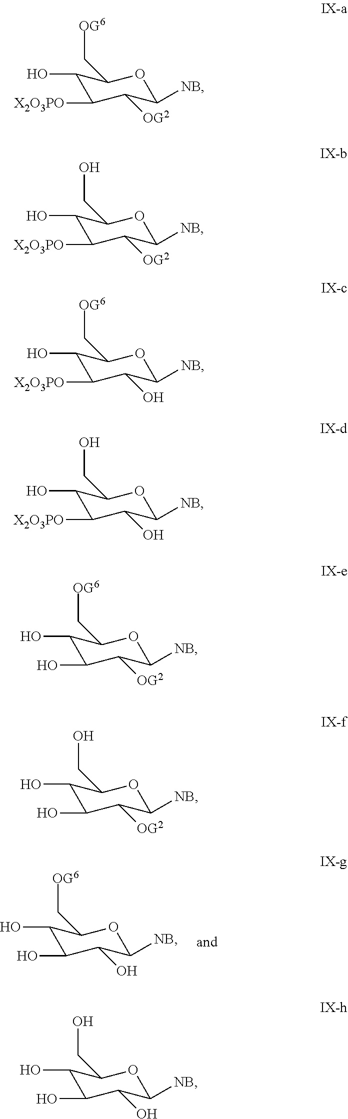

wherein G¹, G², and X are as defined in the preceding embodiments and
wherein —NB comprises an aromatic moiety, a nucleobase, or a derivative or precursor of a nucleobase linked to the glucose through any suitable atom.

45. The compound of embodiment 44, wherein —NB is selected from:

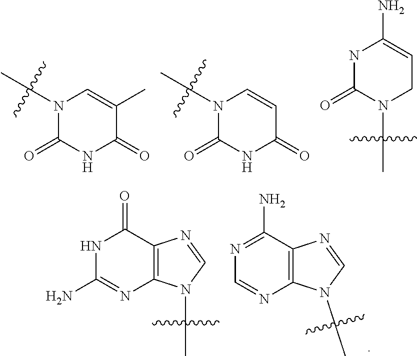

46. The compound of embodiment 44, wherein —NB is selected from:

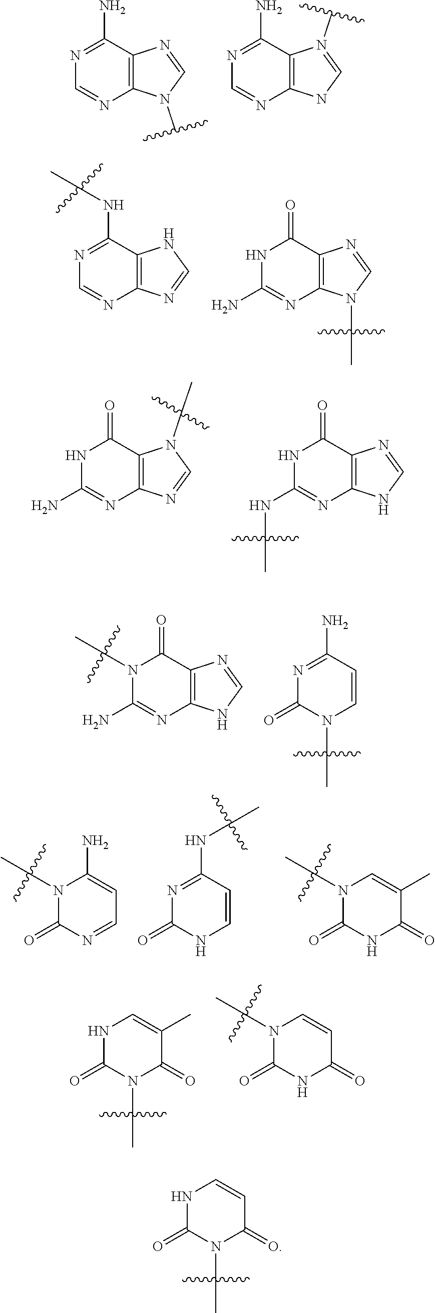

47. The compound of embodiment 44, wherein —NB is selected from:

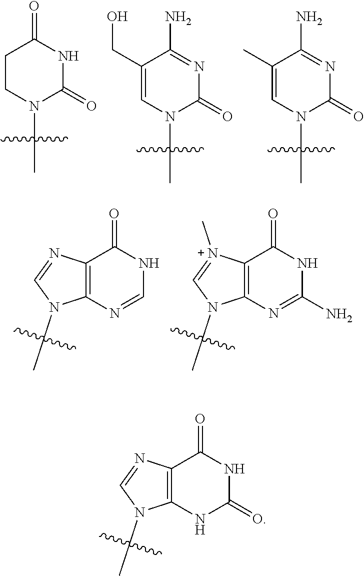

48. The compound of embodiment 44, wherein —NB is selected from:

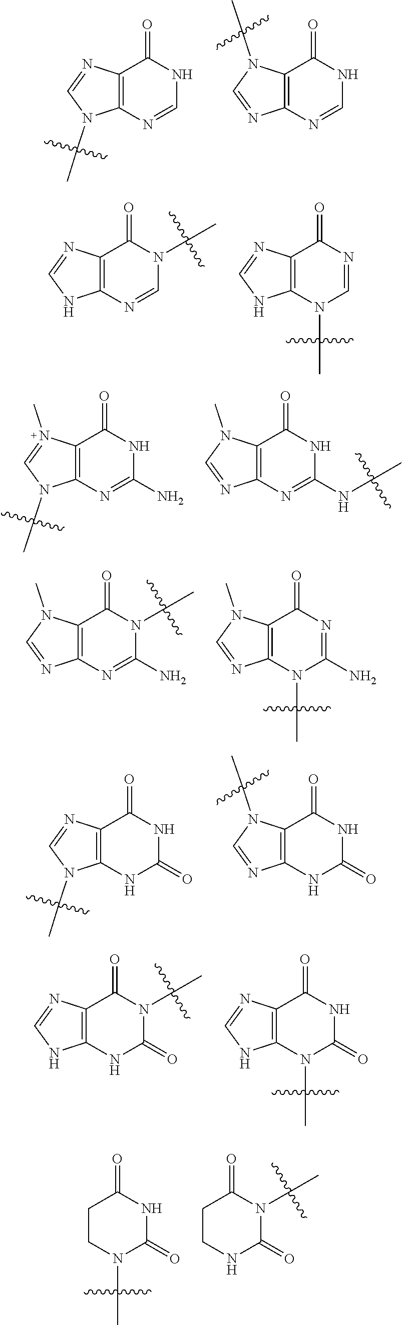

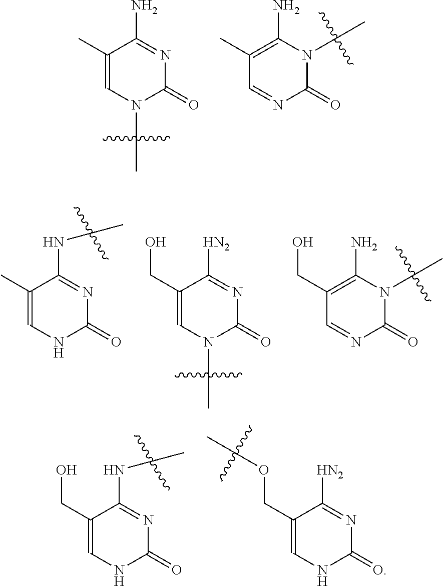

49. A compound having the formulae:

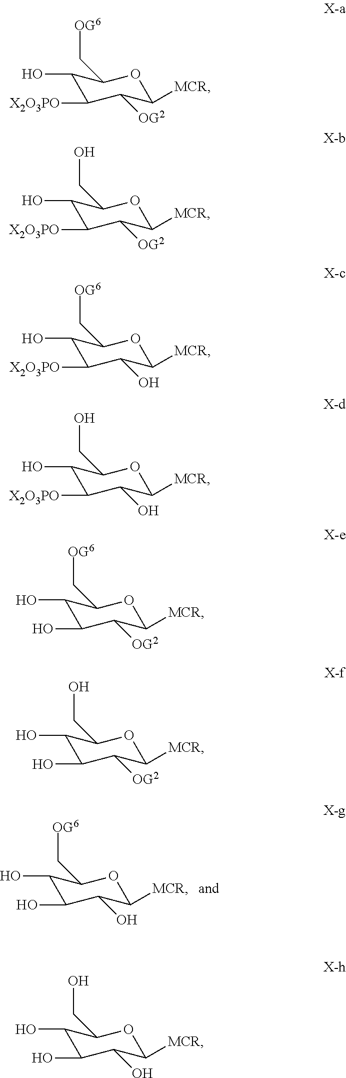

wherein G¹, G², and X are as defined in the preceding embodiments and
wherein -MCR comprises a C₃₋₁₂ alpha beta unsaturated acyl group.

50. The compound of embodiment 49, wherein the moiety -MCR comprises a C₃₋₈ alpha beta unsaturated acyl group.

51. The compound of embodiment 49, wherein the moiety -MCR comprises an acyl group corresponding to an ester of acrylic acid, methylacrylic acid, crotonic acid, methyl crotonic acid, valeric acid, 3-methylcrotonic acid or tiglic acid.

52. The compound of embodiment 49, wherein the moiety -MCR comprises acrylate, methacrylate, or cinnamate.

53. A compound of any one of the preceding embodiments, wherein G¹ is selected from:

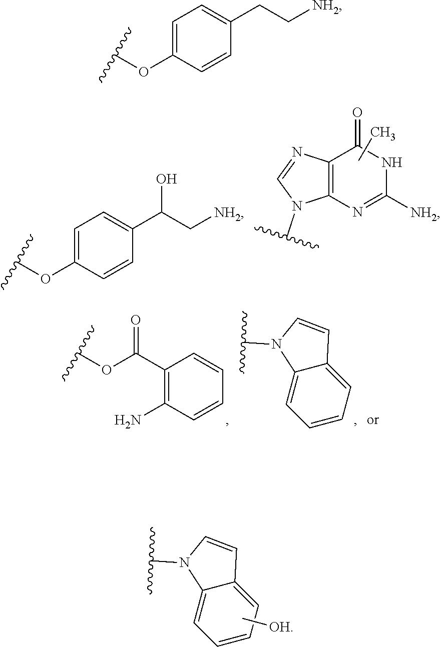

54. A compound of any one of the preceding embodiments, wherein G¹ is selected from:

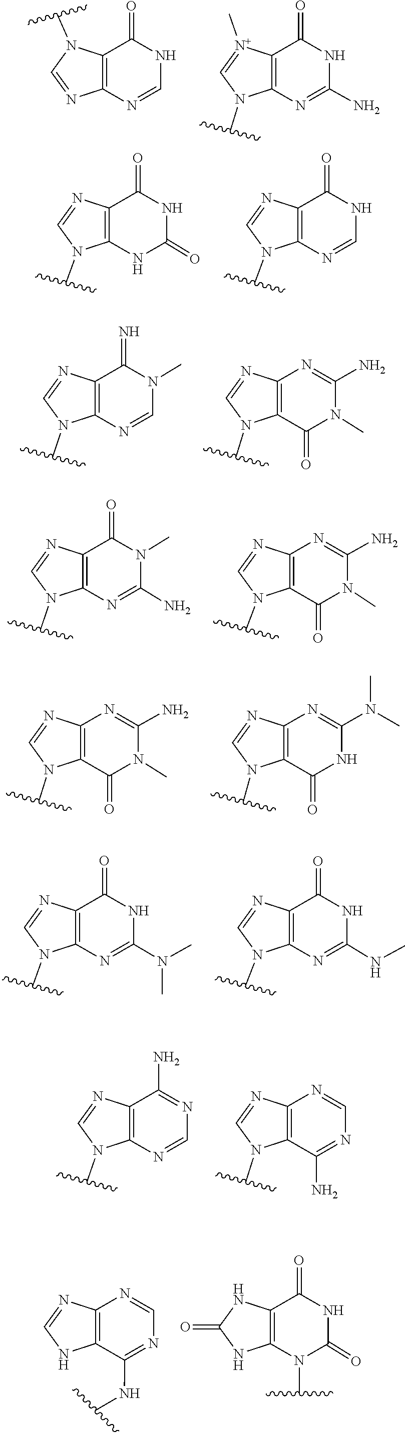

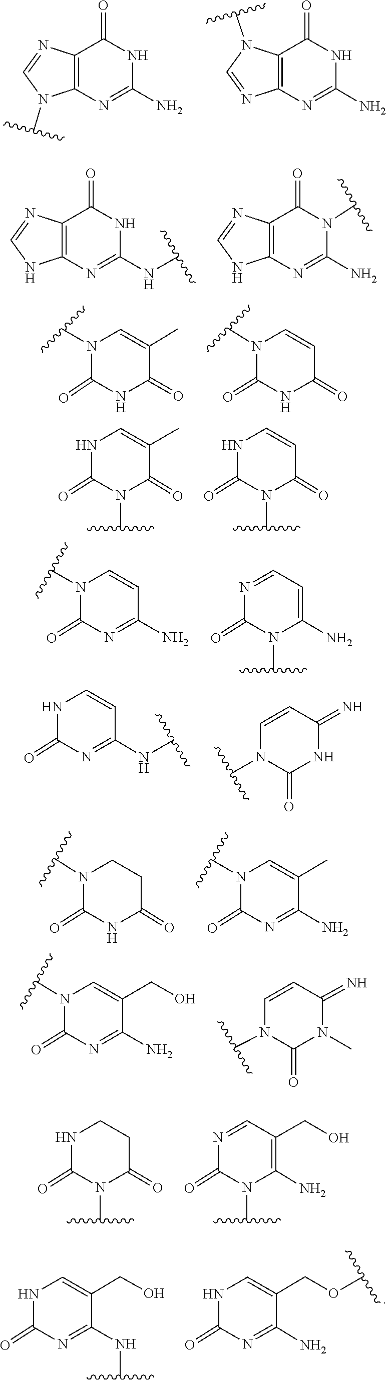

55. A compound of any one of the preceding embodiments, wherein G¹ is selected from:

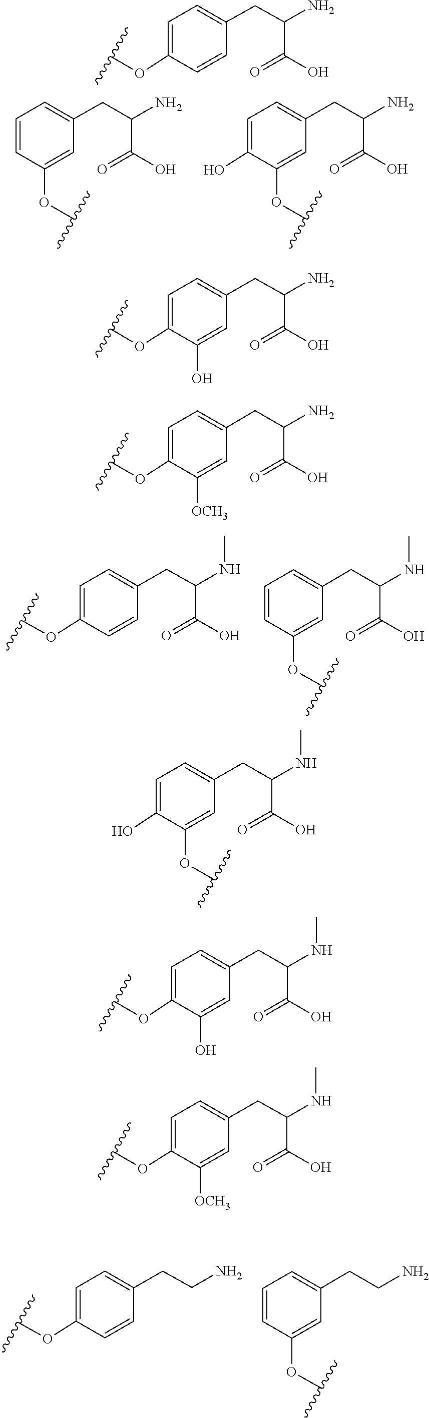

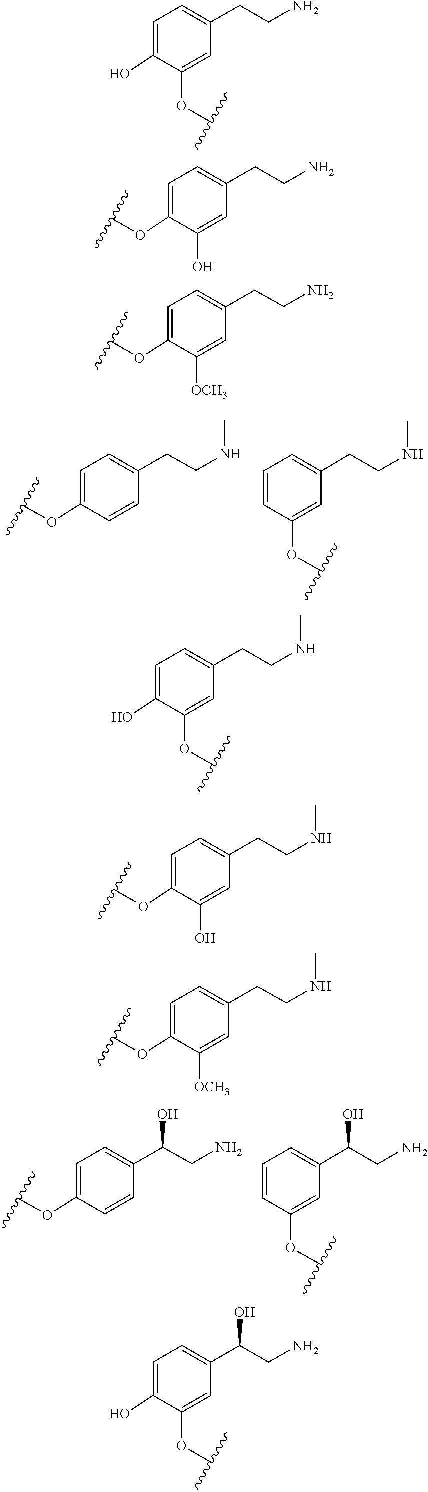

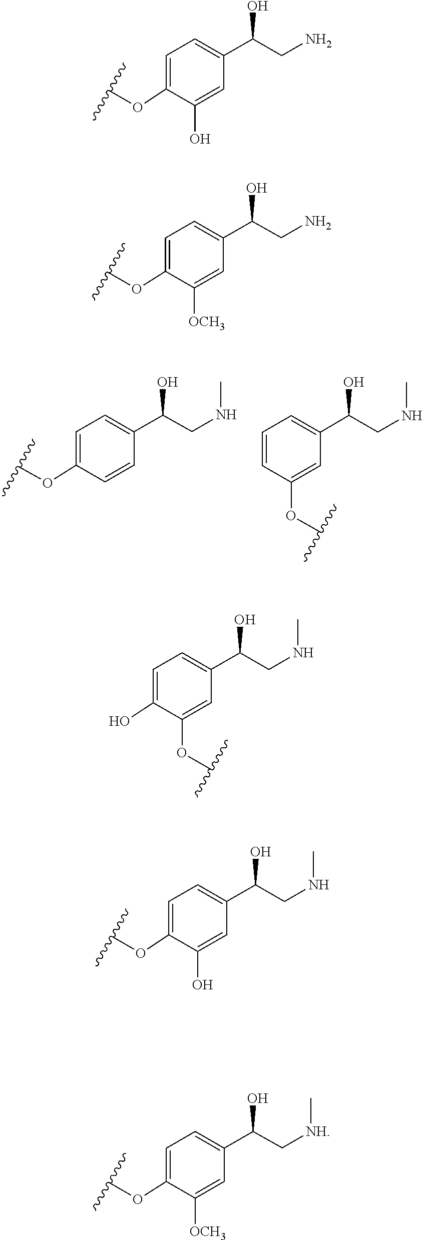

56. A compound of any one of the preceding embodiments, wherein G¹ or —NT is selected from:

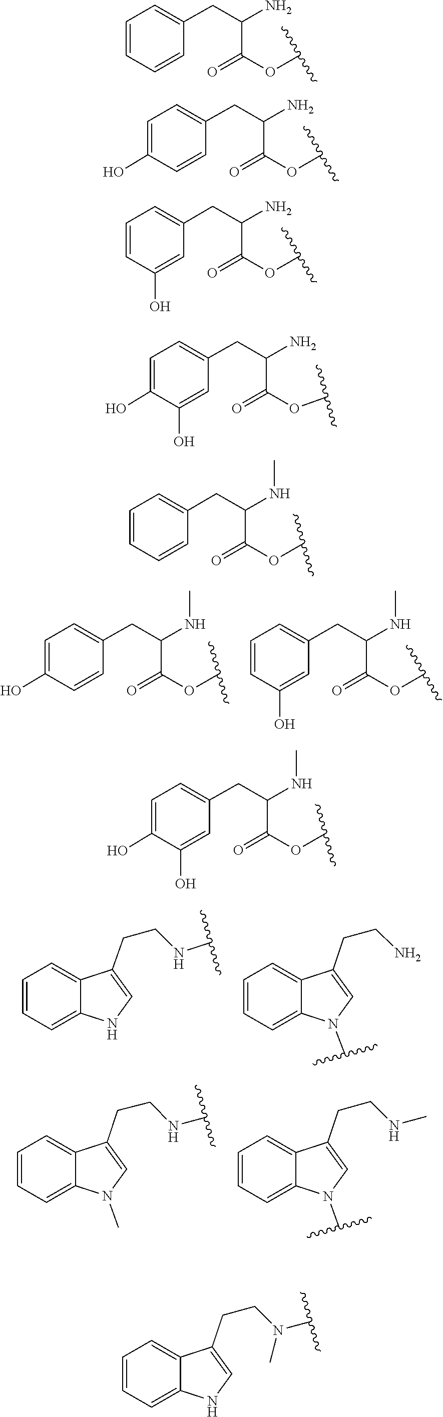

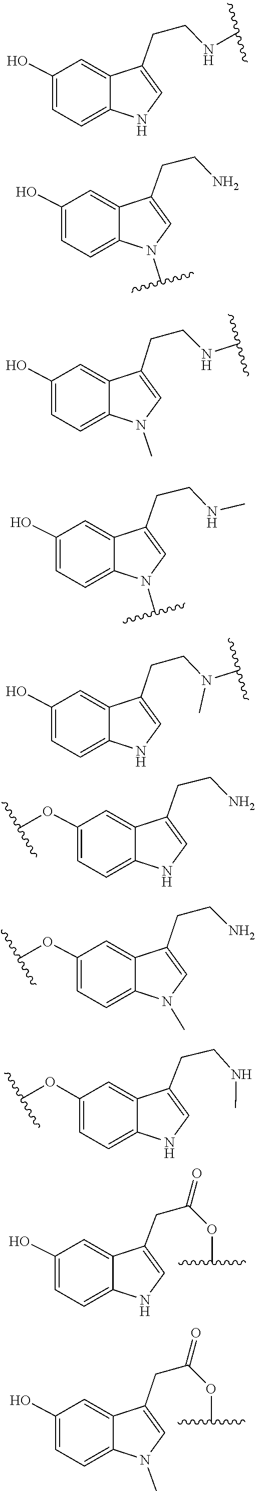

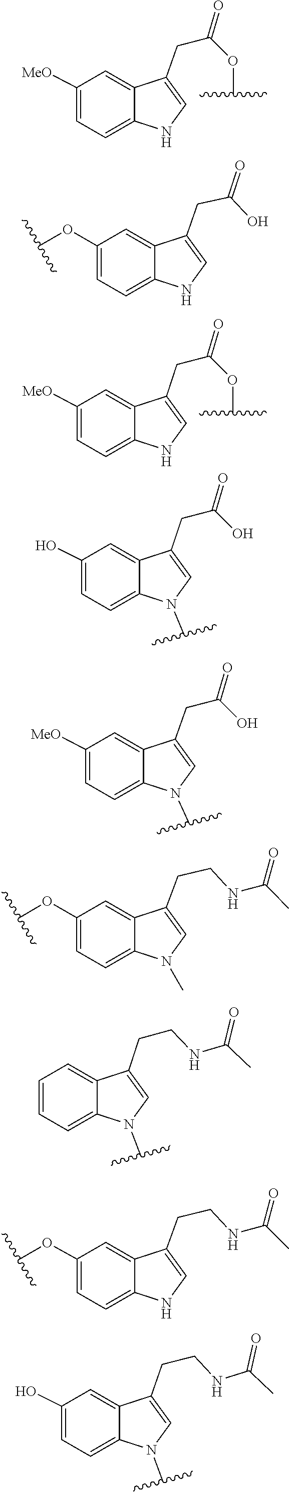

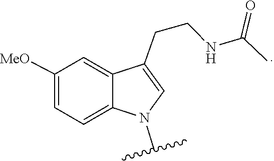 [SMILES: *n1cc(CCNC(C)=O)c2cc(OC)ccc21]

57. A compound of Formulae XI-a, XI-b, XI-c, XI-d, XI-e, XI-f, or XI-g:

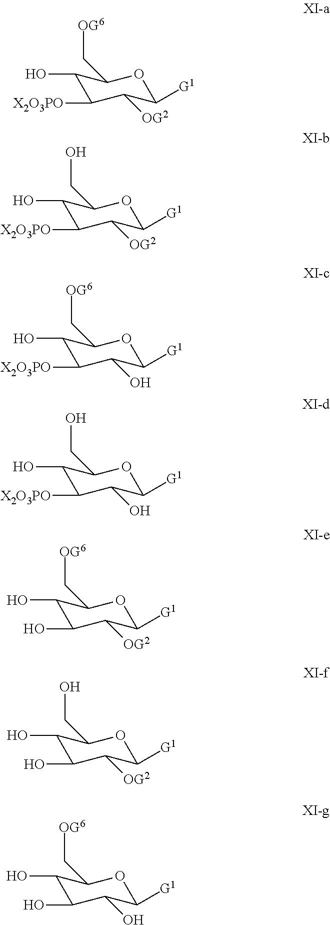

or a pharmaceutically acceptable salt thereof, wherein:
G¹ is —NRⁿ¹Rⁿ², wherein Rⁿ¹ and Rⁿ² are each independently selected from the group consisting of: hydrogen, optionally substituted C₁₋₂₀ aliphatic, optionally substituted C₁₋₂₀ acyl, optionally substituted aryl, and optionally substituted heterocyclic;
G² is an optionally substituted aliphatic, aromatic, heteroaromatic, or heteroaliphatic acyl group;
X is, independently at each occurrence, selected from the group consisting of —H, an optionally substituted phosphate or polyphosphate moiety, M⁺, and Z⁺;
G⁶ is an optionally substituted aliphatic, aromatic, heteroaromatic, or heteroaliphatic acyl group,
R¹⁰ and R¹¹ are each independently selected from the group consisting of: optionally substituted C₁₋₃₂ aliphatic, optionally substituted C₁₋₃₂ heteroaliphatic, optionally substituted aryl, and optionally substituted heteroaryl;
M⁺ is any metal cation; and
Z⁺ is an organic or inorganic ‘onium’ group comprising at least one nitrogen-, phosphorous-, or sulfur-based cation.

58. The compound of any one of the preceding embodiments, wherein Rⁿ¹ is hydrogen and Rⁿ² is optionally substituted C₁₋₁₂ aliphatic.

59. The compound of any one of the preceding embodiments, wherein Rⁿ¹ is hydrogen and Rⁿ² is optionally substituted C₁₋₆ aliphatic.

60. The compound of any one of the preceding embodiments, wherein Rⁿ¹ is methyl and Rⁿ² is optionally substituted C₁₋₆ aliphatic.

61. The compound of any one of the preceding embodiments, wherein Rⁿ¹ is hydrogen and Rⁿ² is optionally substituted aryl.

62. The compound of any one of the preceding embodiments, Rⁿ¹ is methyl and Rⁿ² is optionally substituted aryl.

63. The compound of any one of the preceding embodiments, wherein Rⁿ¹ is hydrogen and R² is optionally substituted C₃₋₁₂ heterocyclic.

64. The compound of any one of the preceding embodiments, wherein Rⁿ¹ is hydrogen and R² is optionally substituted C₃₋₁₂ heterocyclic.

65. The compound of any one of the preceding embodiments, wherein Rⁿ¹ is methyl and Rⁿ² is optionally substituted C₃₋₆ heterocyclic.

66. The compound of any one of the preceding embodiments, wherein Rⁿ¹ is hydrogen and Rⁿ² is optionally substituted acyl.

67. The compound of any one of the preceding embodiments, wherein Rⁿ¹ is hydrogen and Rⁿ² is optionally substituted acyl.

68. The compound of any one of the preceding embodiments, wherein Rⁿ¹ is methyl and Rⁿ² is optionally substituted acyl.

69. The compound of any one of the preceding embodiments, wherein —NRⁿ¹Rⁿ² comprises a monoamine neurotransmitter or a derivative or precursor thereof

70. The compound of any one of the preceding embodiments, wherein —NRⁿ¹Rⁿ² comprises a catecholamine neurotransmitter or a derivative or precursor thereof.

71. The compound of any one of the preceding embodiments, wherein —NRⁿ¹Rⁿ² is selected from the group consisting of: dopamine, norepinepherine, epinepherine, histamine, and serotonin.

72. The compound of any one of the preceding embodiments, wherein —NRⁿ¹Rⁿ² is selected from tryptamine, phenethylamine, N-methylphenethylamine, phenethanolamine, m-tyramine, p-tyramine, 3-methoxytyramine, N-methyltyramine, 3-indothyronamine, m-octopamine, p-octopamine, and synepherine.

73. The compound of any one of the preceding embodiments, wherein G¹ is selected from:

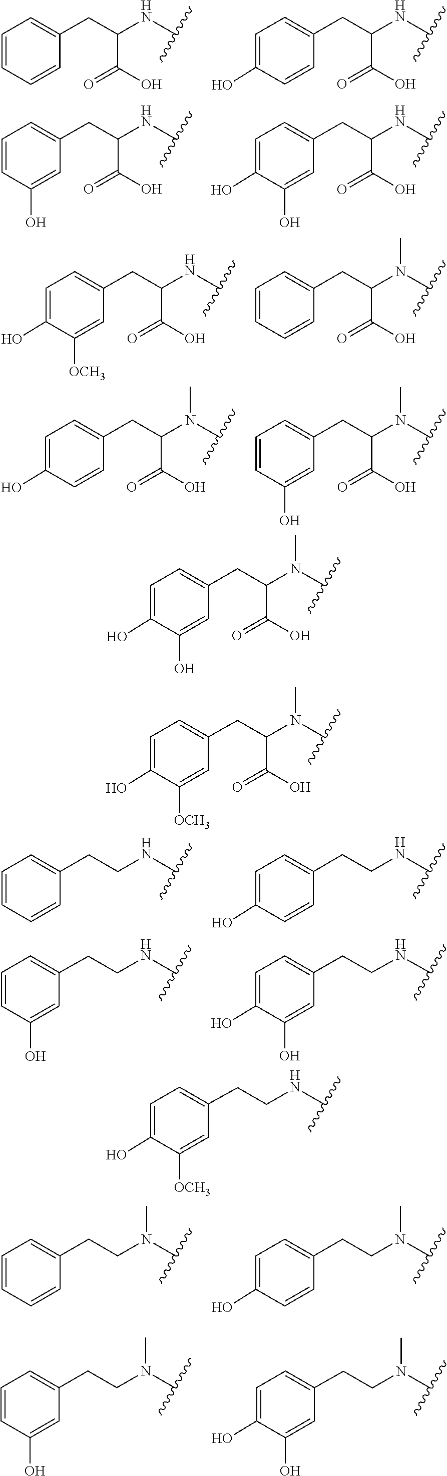

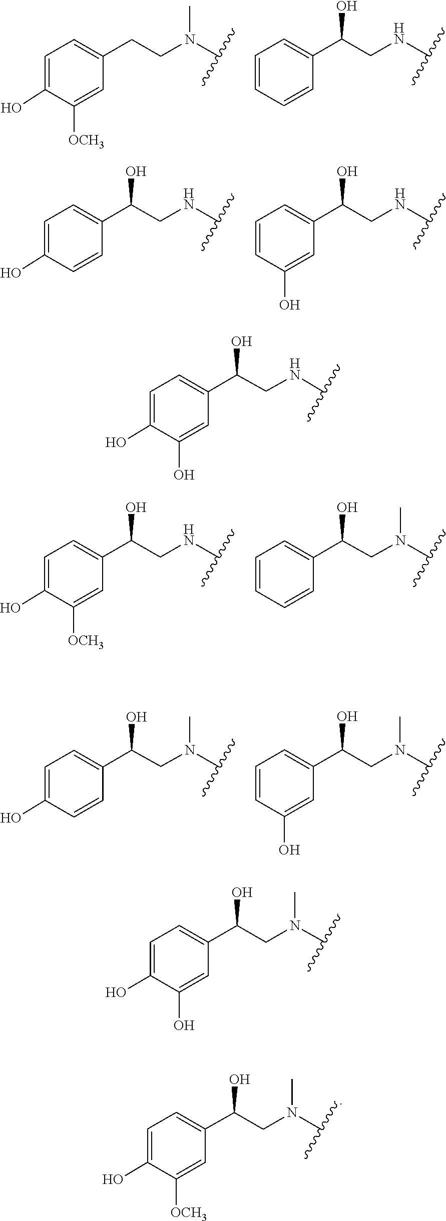

74. A compound of any one of the preceding embodiments, wherein G¹ or —NT is selected from:

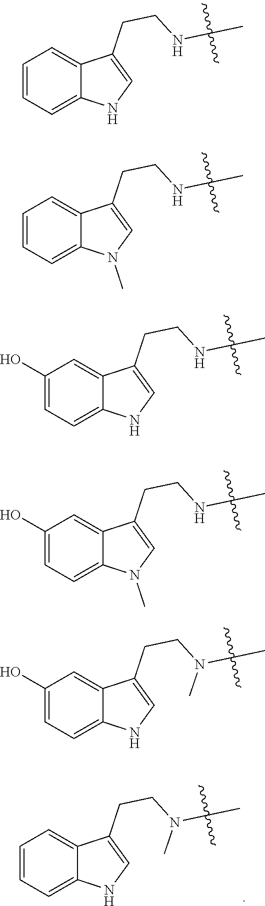

75. The compound of any one of the preceding embodiments, wherein G² is selected from:

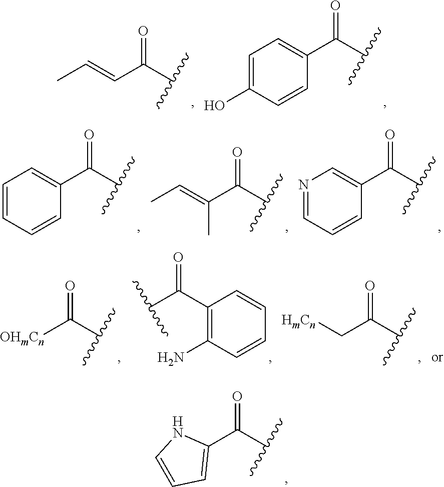

wherein n is 1-35 (e.g., 1-24, 1-18, 1-12, 1-8, or 1-6) and m is an integer dependent upon n to provide a stable saturated, unsaturated, or polyunsaturated aliphatic group.

76. The compound of any one of the preceding embodiments, wherein G² is selected from:

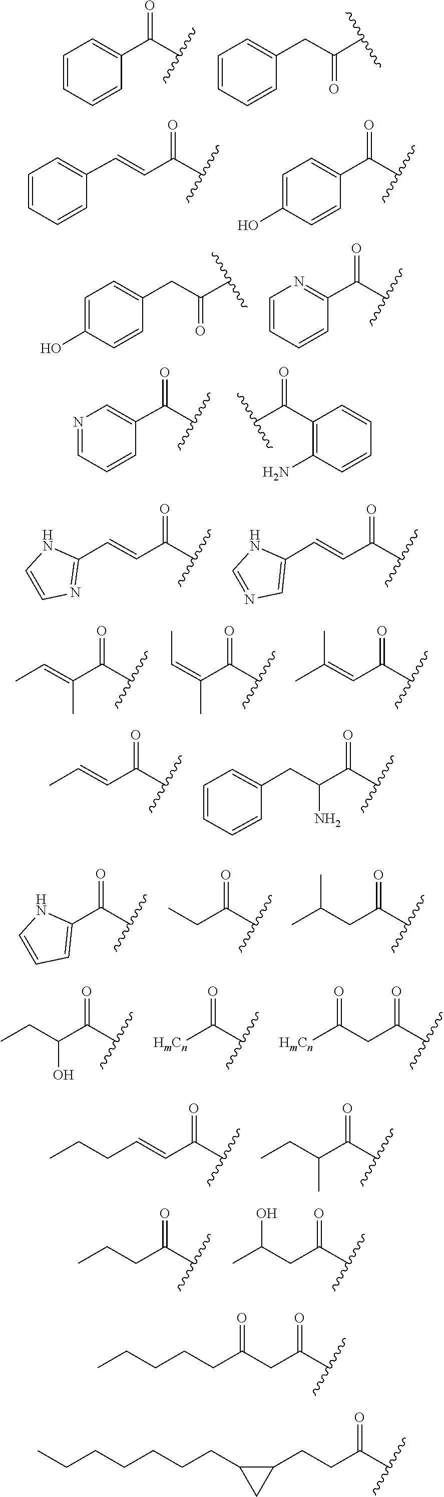

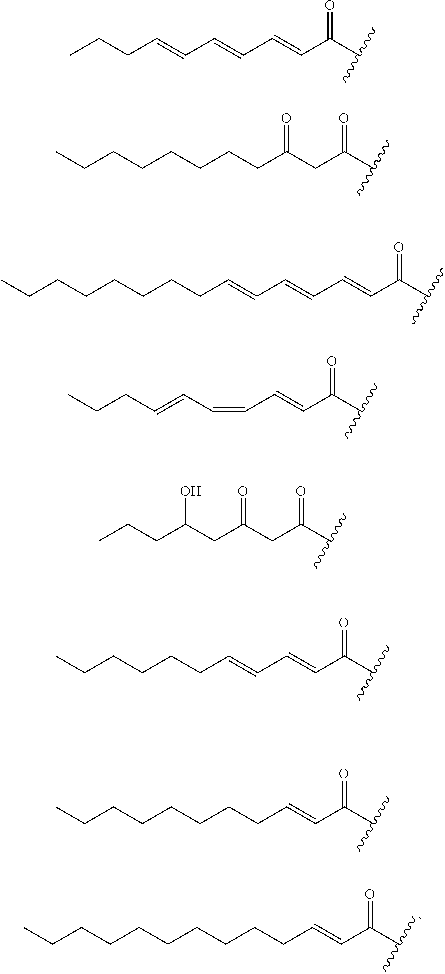

wherein n is 1-35 (e.g., 1-24, 1-18, 1-12, 1-8, or 1-6) and m is an integer dependent upon n to provide a stable saturated, unsaturated, or polyunsaturated aliphatic group.

77. The compound of any one of the preceding embodiments, wherein G² is selected from:

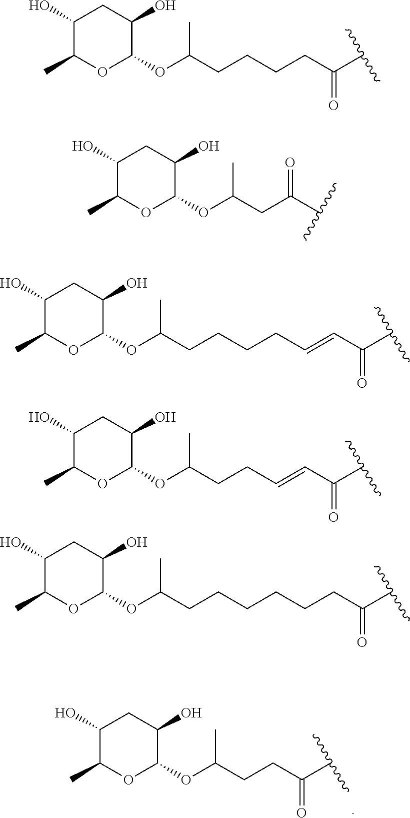 [SMILES: *C(=O)CCCCC(C)O[C@@H]1O[C@@H](C)[C@H](O)C[C@H]1O ; *C(=O)CC(C)O[C@@H]1O[C@@H](C)[C@H](O)C[C@H]1O ; *C(=O)/C=C/CCCCC(C)O[C@@H]1O[C@@H](C)[C@H](O)C[C@H]1O ; *C(=O)/C=C/CCC(C)O[C@@H]1O[C@@H](C)[C@H](O)C[C@H]1O ; *C(=O)CCCCCCC(C)O[C@@H]1O[C@@H](C)[C@H](O)C[C@H]1O ; *C(=O)CCC(C)O[C@@H]1O[C@@H](C)[C@H](O)C[C@H]1O]

78. The compound of any one of the preceding embodiments, wherein each X is hydrogen.

79. The compound of any one of the preceding embodiments, wherein one X is hydrogen and the other X is a phosphate or diphosphate.

80. The compound of any one of the preceding embodiments, wherein one X is hydrogen and the other X is M⁺.

81. The compound of any one of the preceding embodiments, wherein one X is hydrogen and the other X is Z⁺.

82. The compound of any one of the preceding embodiments, wherein G⁶ is selected from:

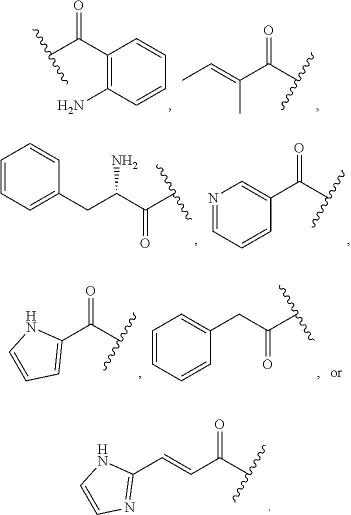

83. The compound of any one of the preceding embodiments, wherein G⁶ is selected from:

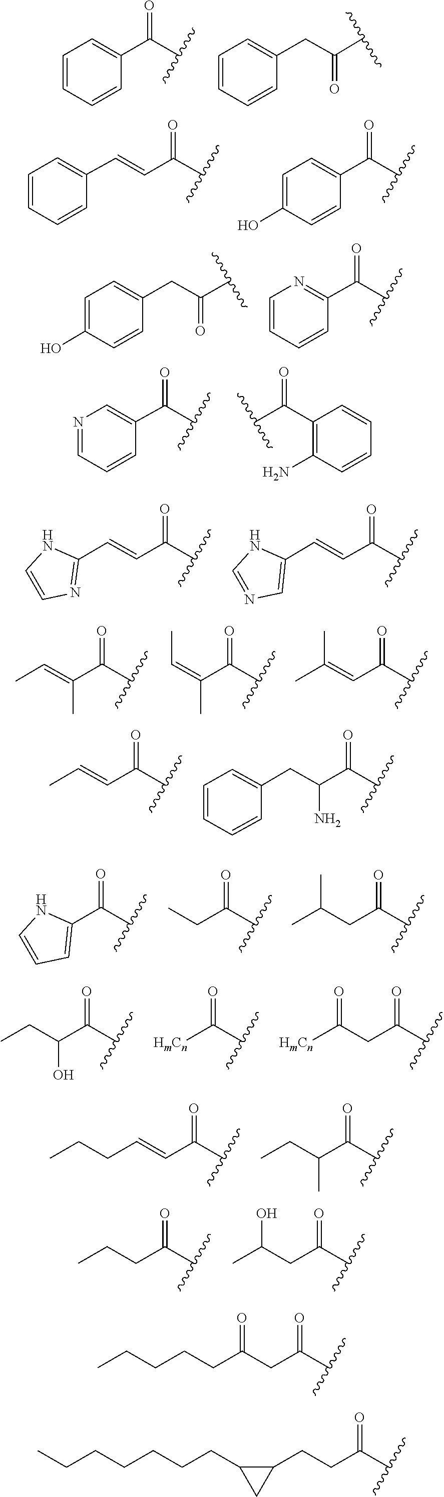

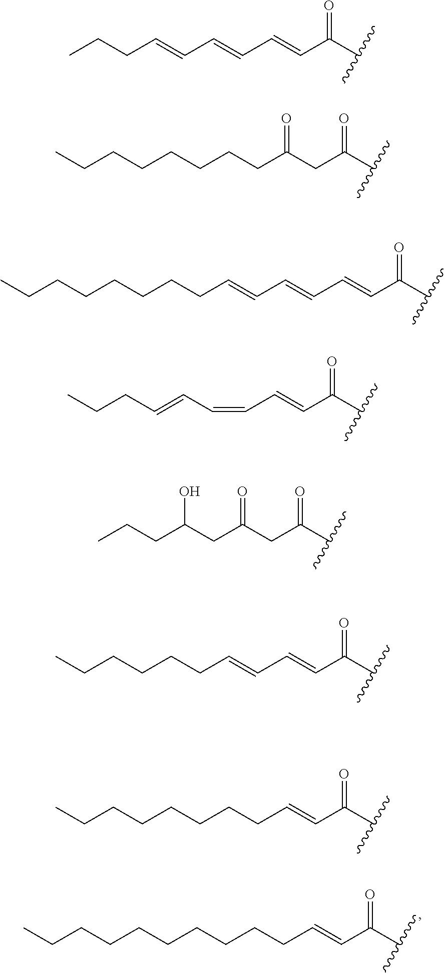

wherein n is 1-35 (e.g., 1-24, 1-18, 1-12, 1-8, or 1-6) and m is an integer dependent upon n to provide a stable saturated, unsaturated, or polyunsaturated aliphatic group.

84. The compound of any one of the preceding embodiments, wherein G⁶ is selected from:

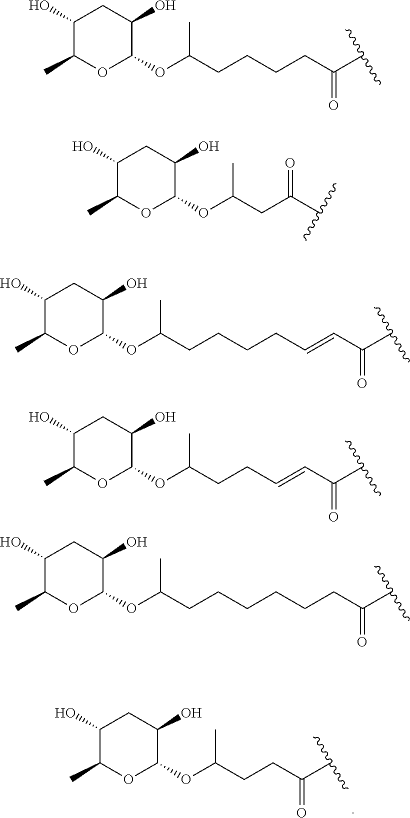 [SMILES: *C(=O)CCCCC(C)O[C@@H]1O[C@@H](C)[C@H](O)C[C@H]1O ; *C(=O)CC(C)O[C@@H]1O[C@@H](C)[C@H](O)C[C@H]1O ; *C(=O)/C=C/CCCCC(C)O[C@@H]1O[C@@H](C)[C@H](O)C[C@H]1O ; *C(=O)/C=C/CCC(C)O[C@@H]1O[C@@H](C)[C@H](O)C[C@H]1O ; *C(=O)CCCCCCC(C)O[C@@H]1O[C@@H](C)[C@H](O)C[C@H]1O ; *C(=O)CCC(C)O[C@@H]1O[C@@H](C)[C@H](O)C[C@H]1O]

85. The compound of any one of the preceding embodiments, wherein G² and G⁶ are hydrogen.

86. A compound of Formula A-1 or A-2:

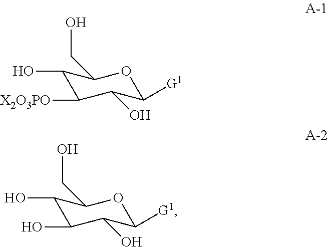

or a pharmaceutically acceptable salt thereof,
G¹ is an optionally substituted moiety selected from the group consisting of: N-linked heteroaryl, —OR¹⁰ and —OC(O)R¹¹;
X is, independently at each occurrence, selected from the group consisting of —H, an optionally substituted phosphate or polyphosphate moiety, M⁺, and Z⁺;
R¹⁰ and R¹¹ are each independently selected from the group consisting of: optionally substituted C₁₋₃₂ aliphatic, optionally substituted C₁₋₃₂ heteroaliphatic, optionally substituted aryl, and optionally substituted heteroaryl;
M⁺ is any metal cation; and
Z⁺ is an organic or inorganic ‘onium’ group comprising at least one nitrogen-, phosphorous-, or sulfur-based cation.

87. The compound of any one of the preceding embodiments, wherein X is hydrogen.

88. The compound of any one of the preceding embodiments, wherein G¹ is —OR¹⁰.

89. The compound of any one of the preceding embodiments, wherein R¹⁰ is optionally substituted aryl or optionally substituted heteroaryl.

90. The compound of any one of the preceding embodiments, wherein R¹⁰ is substituted with a group containing a nitrogen atom.

91. The compound of any one of the preceding embodiments, wherein R¹⁰ is substituted with an amino (—NH₂) group.

92. The compound of any one of the preceding embodiments, wherein R¹⁰ is optionally indole (e.g., indole substituted with —(CH₂)₀₋₄N(R^{∘})C(O)R^{∘}).

93. The compound of any one of the preceding embodiments, wherein R¹⁰ is aryl or heteroaryl substituted with —(CH₂)₀₋₄N(R^{∘})₂, —(CH₂)₀₋₄N(R^{∘})C(O)R^{∘}, or —(CH₂)₀₋₄C(O)N(R^{∘})₂.

94. The compound of any one of the previous embodiments, wherein G¹ is an N-linked nucleobase.

95. The compound of any one of the previous embodiments, wherein G¹ is selected from the group consisting of:

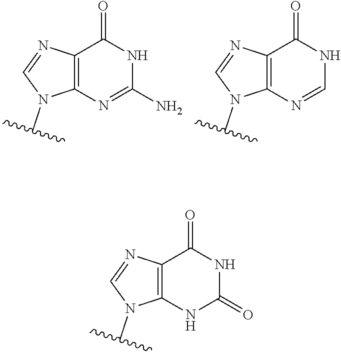 [SMILES: *n1cnc2c(=O)[nH]c(N)nc21 ; *n1cnc2c(=O)[nH]cnc21 ; *n1cnc2c(=O)[nH]c(=O)[nH]c21]

which may be optionally substituted as allowed by valency.

96. The compound of any one of the previous embodiments, wherein G¹ is:

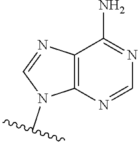

wherein the G¹ is substituted at any position allowed by valency.

97. The compound of any one of the previous embodiments, wherein G¹ is selected from the group consisting of:

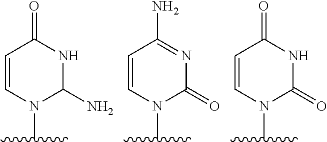

which may be optionally substituted as allowed by valency.

98. The compound of any one of the previous embodiments, wherein G¹ is other than unsubstituted adenine.

99. The compound of any one of the previous embodiments, wherein G¹ is —OR¹⁰.

100. The compound of any one of the previous embodiments, wherein G¹ is —OR¹⁰ and R¹⁰ is optionally substituted aryl or optionally substituted heteroaryl.

101. The compound of any one of the previous embodiments, wherein G¹ is —OR¹⁰ and R¹⁰ is optionally substituted phenyl, optionally substituted 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur.

102. The compound of any one of the previous embodiments, wherein R¹⁰ is substituted with a group containing a nitrogen atom.

103. The compound of any one of the previous embodiments, wherein R¹⁰ is substituted with an amino (—NH₂) group.

104. The compound of any one of the previous embodiments, wherein R¹⁰ is substituted with —(CH₂)₀₋₄N(R^{∘})₂, —(CH₂)₀₋₄N(R^{∘})C(O)R^{∘}, —(CH₂)₀₋₄C(O)N(R^{∘})₂.

105. The compound of any one of the previous embodiments, wherein G¹ is —OC(O)R¹¹.

106. The compound of any one of the previous embodiments, wherein G¹ is —OC(O)R¹¹ and R¹¹ is optionally substituted aryl or optionally substituted heteroaryl.

107. The compound of any one of the previous embodiments, wherein G¹ is —OC(O)R¹¹ and R¹¹ is optionally substituted phenyl, optionally substituted 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur.

108. The compound of any one of the previous embodiments, wherein R¹¹ is substituted with a group containing a nitrogen atom.

109. The compound of any one of the previous embodiments, wherein R¹¹ is substituted with an amino (—NH₂) group.

110. The compound of any one of the previous embodiments, wherein R¹¹ is substituted with —(CH₂)₀₋₄N(R^{∘})₂, —(CH₂)₀₋₄N(R^{∘})C(O)R^{∘}, —(CH₂)₀₋₄C(O)N(R^{∘})₂.

111. The compound of any one of the preceding embodiments, wherein R¹⁰ and R¹¹ do not comprise a nitro group.

112. The compound of any one of the preceding embodiments, wherein G¹ does not comprise a pyrrole or indole.

113. The compound of any one of the preceding embodiments, wherein G² and G⁶ are not acetyl or benzoyl.

114. The compound of any one of the preceding embodiments, wherein the compound 5 is other than one or more more of the following compounds:

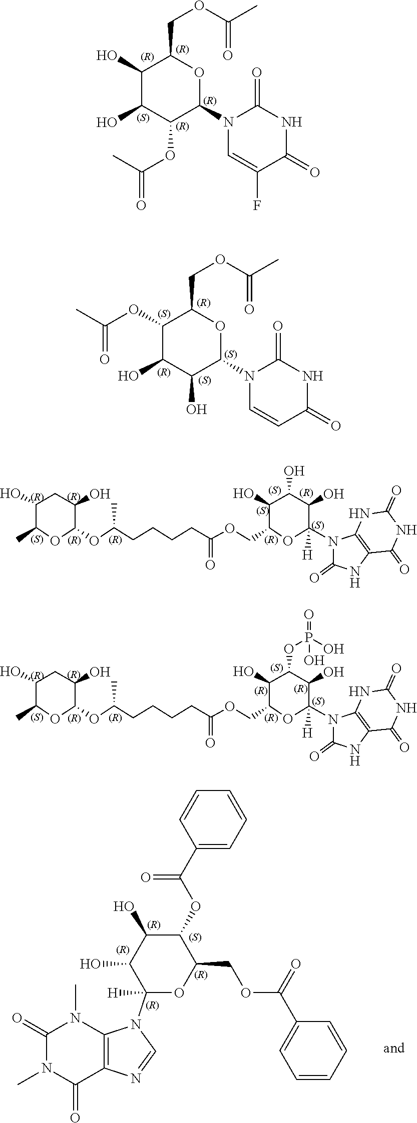

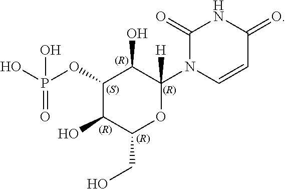

115. The compound of any one of the preceding embodiments, wherein the compound 5 is other than one of more of the following compounds:

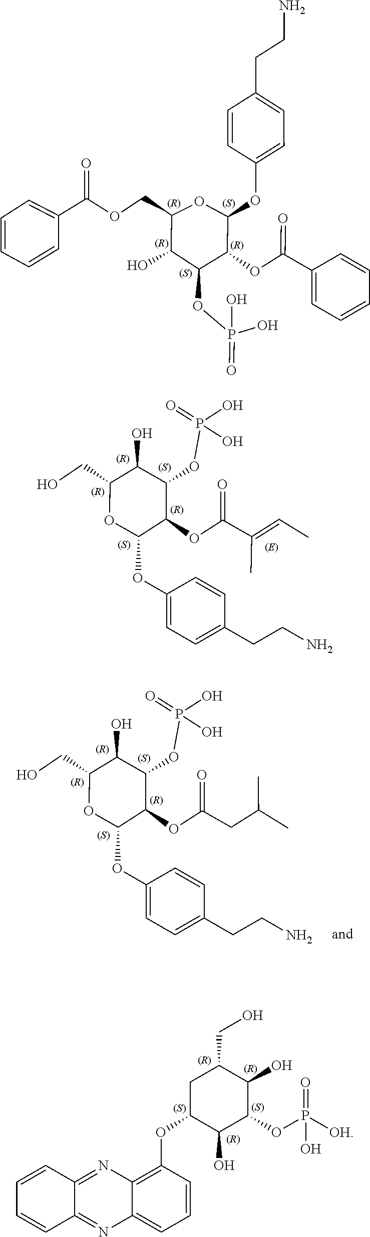

116. The compound of any one of the preceding embodiments, wherein the compound is other than one or more of the following compounds:

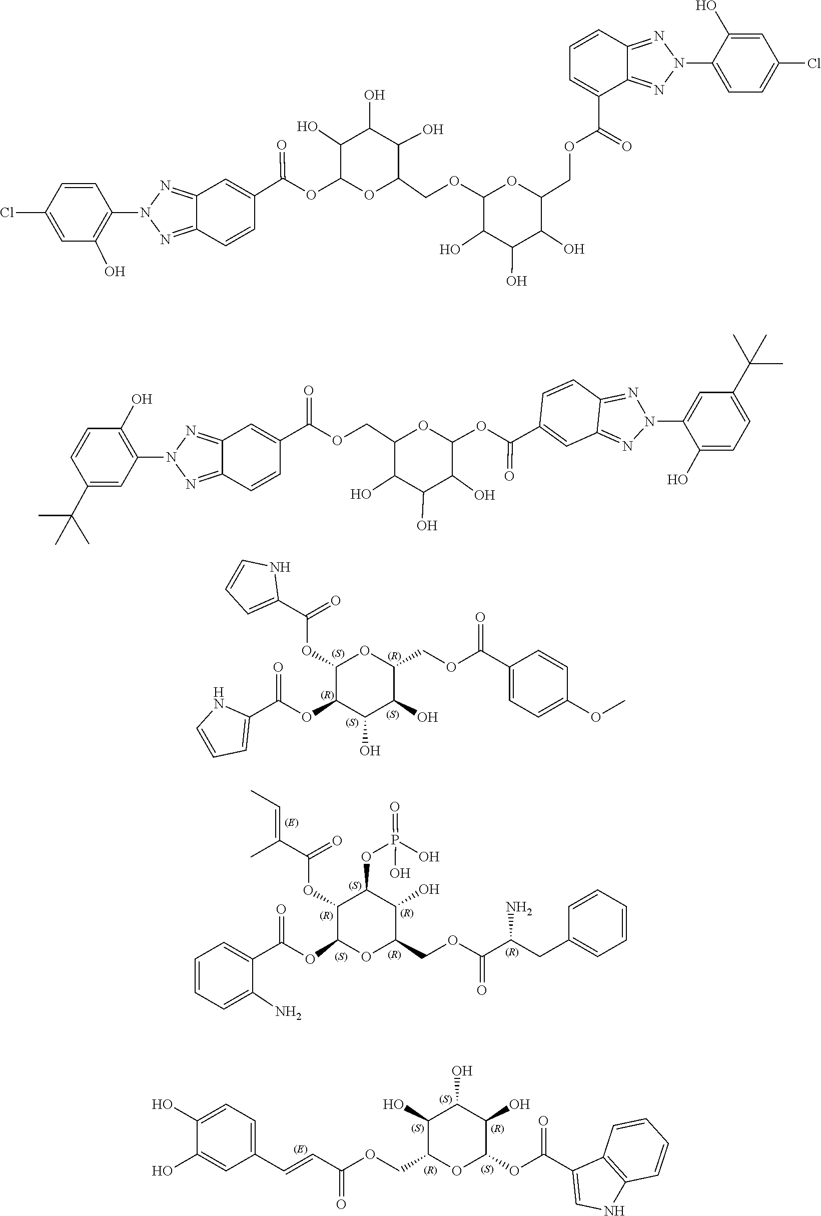

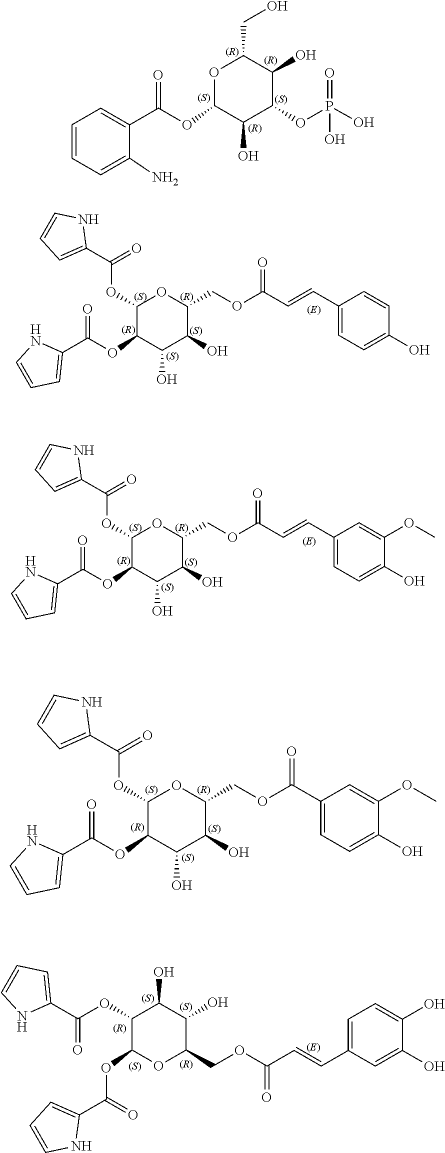

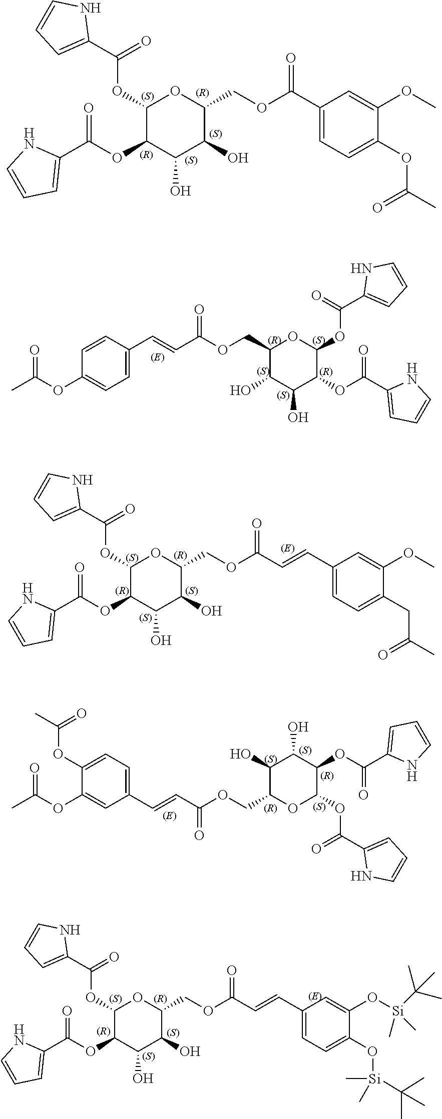

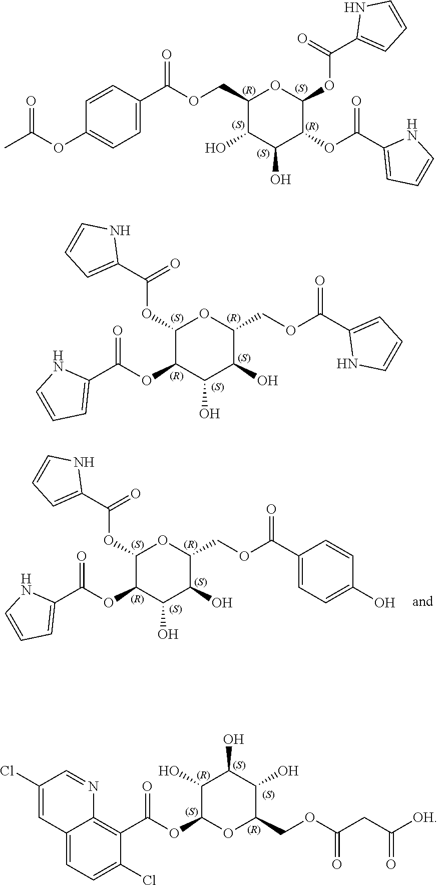

117. A compound of Table S4a, or a pharmaceutically acceptable salt thereof.

118, A compound of Table S4b, or a pharmaceutically acceptable salt thereof.

119. A compound of Table S5, or a pharmaceutically acceptable salt thereof.

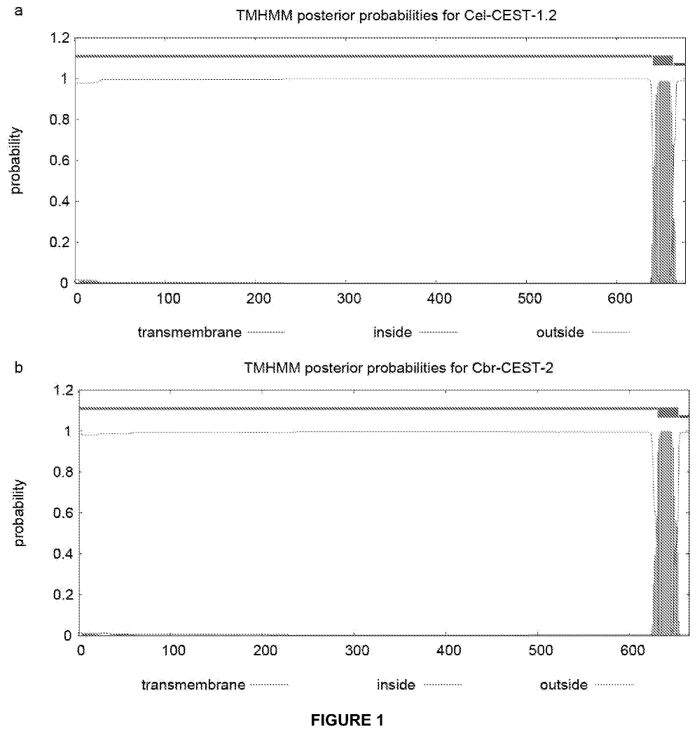

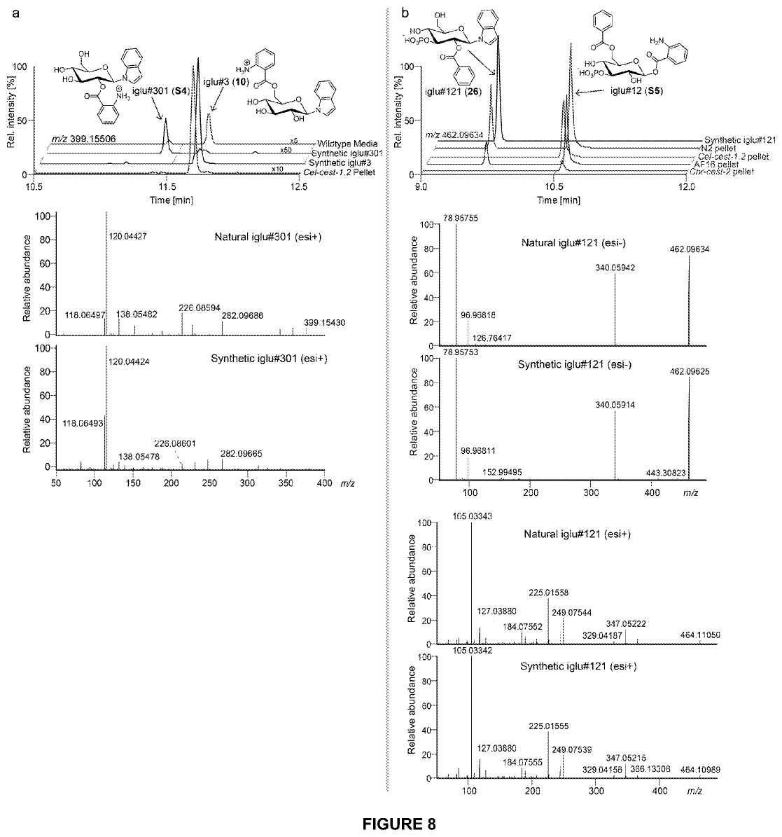

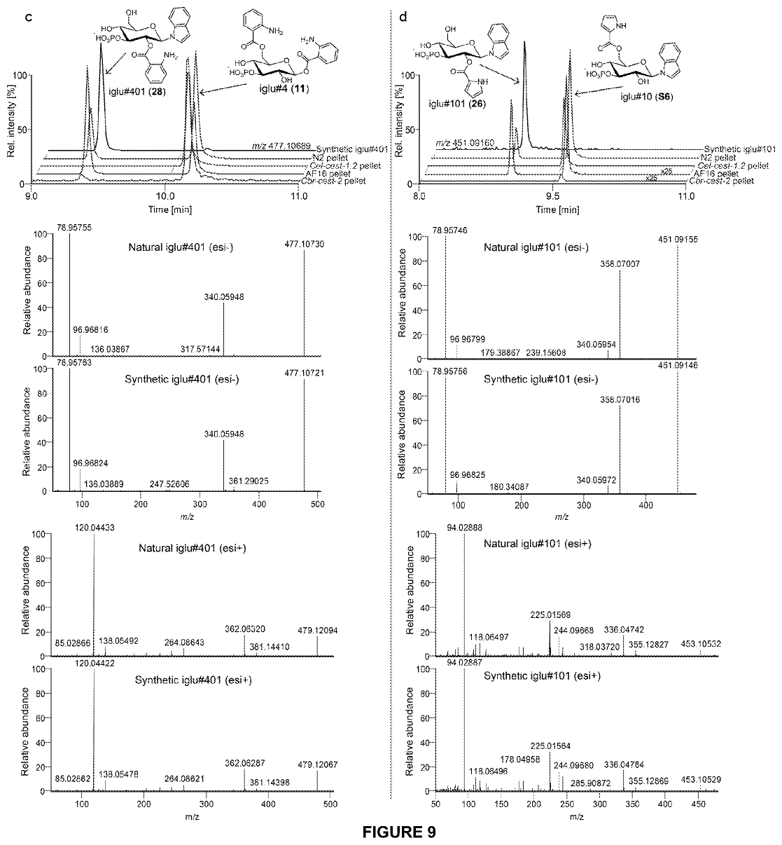

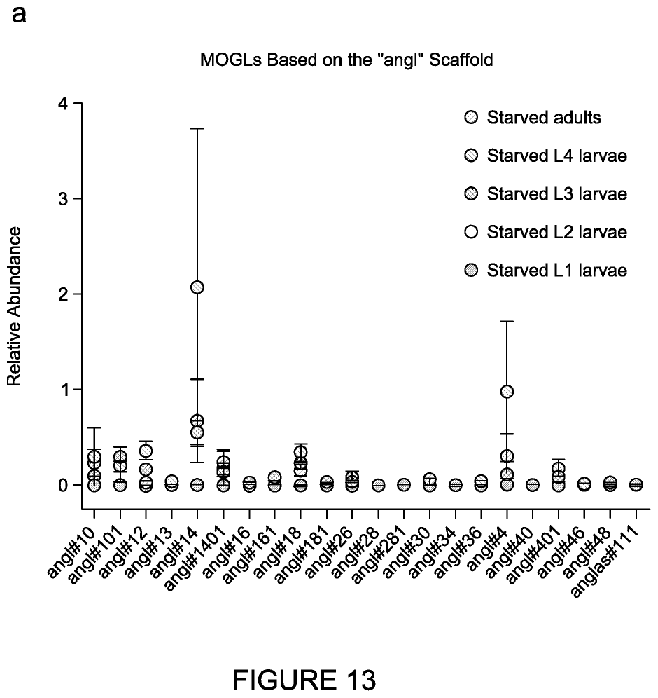

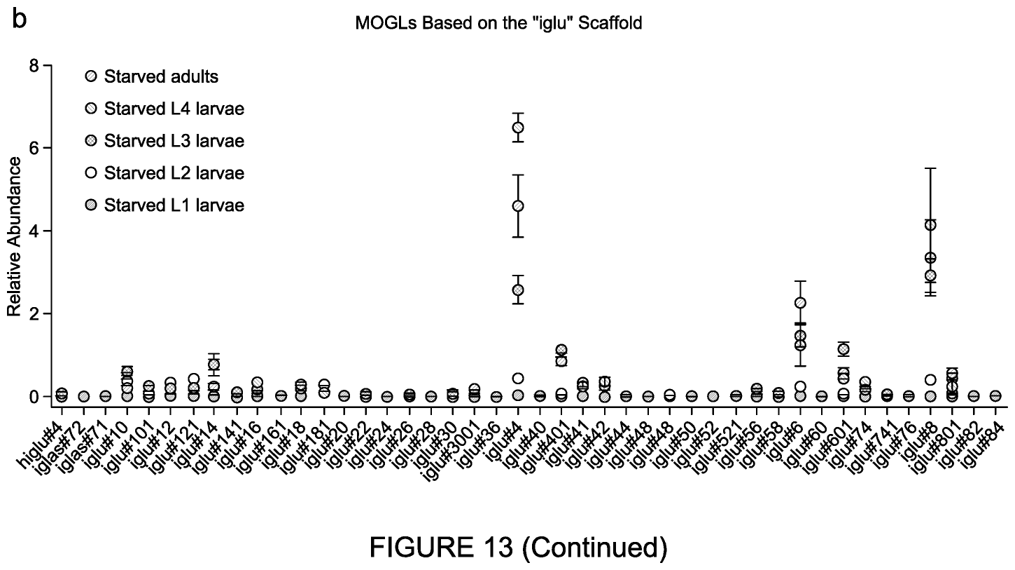

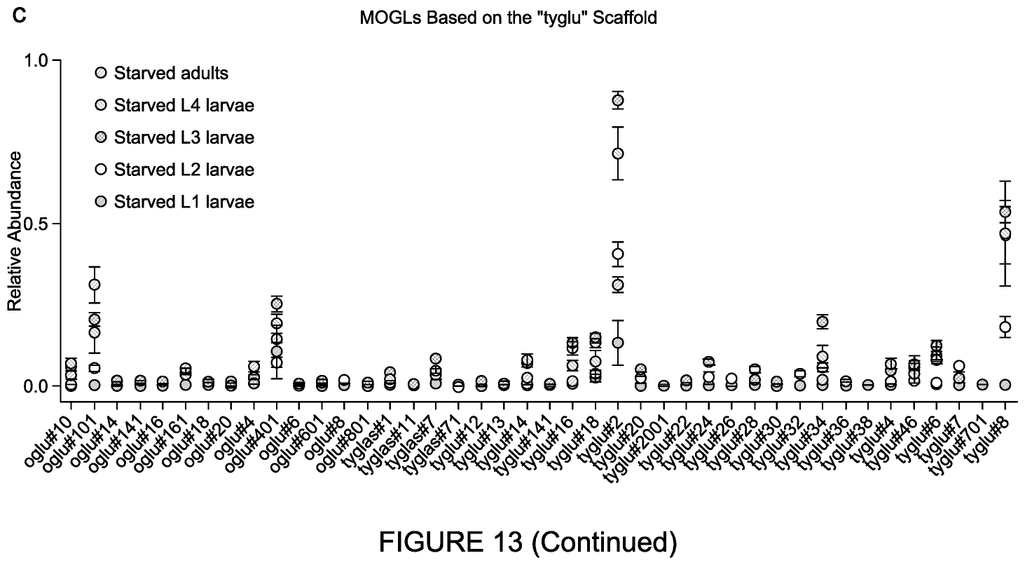

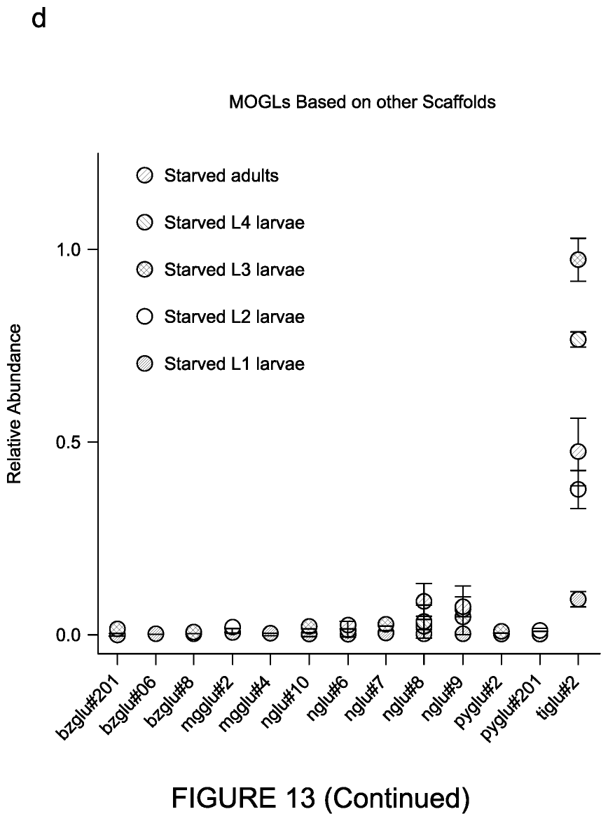

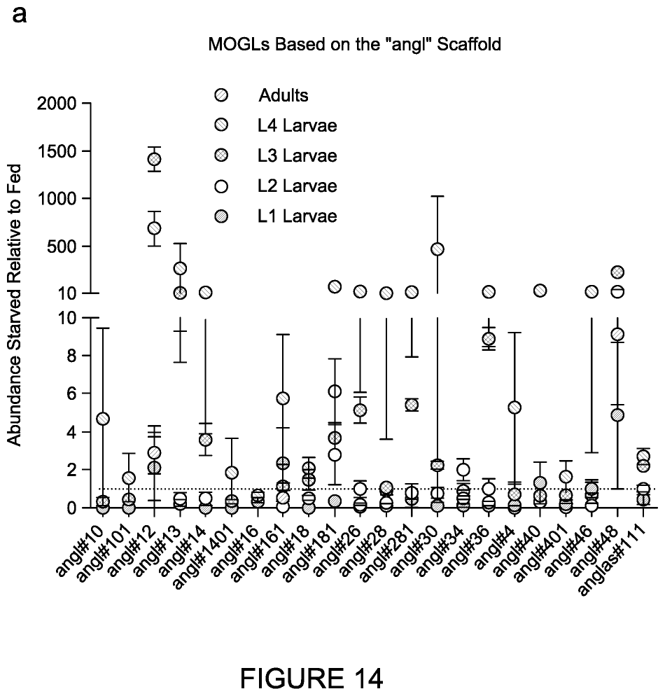

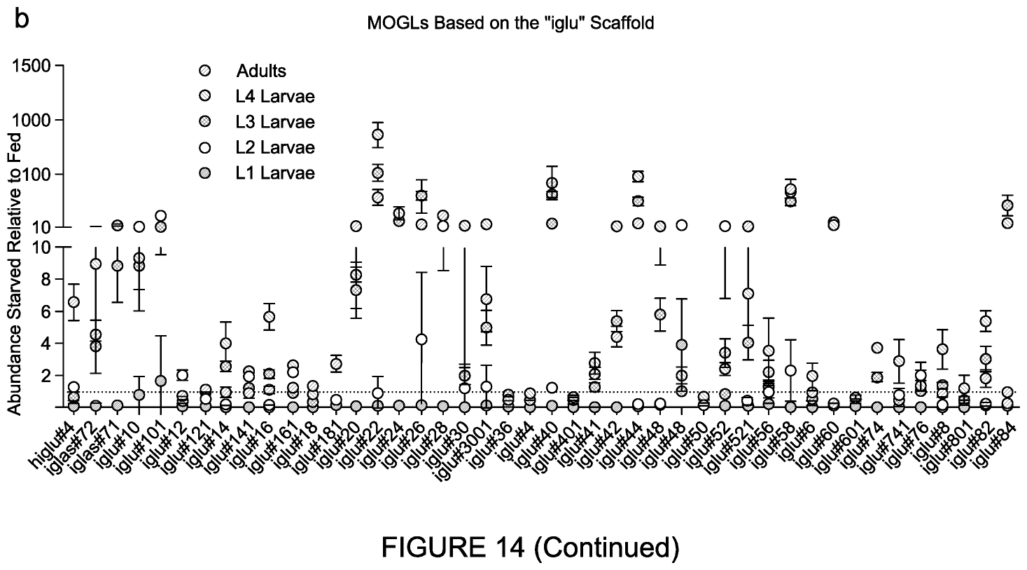

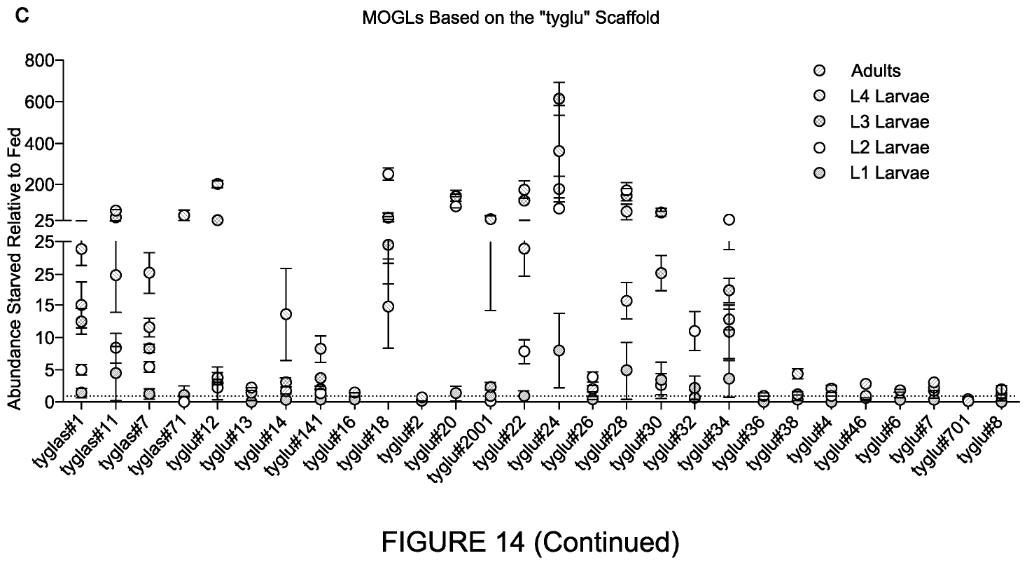

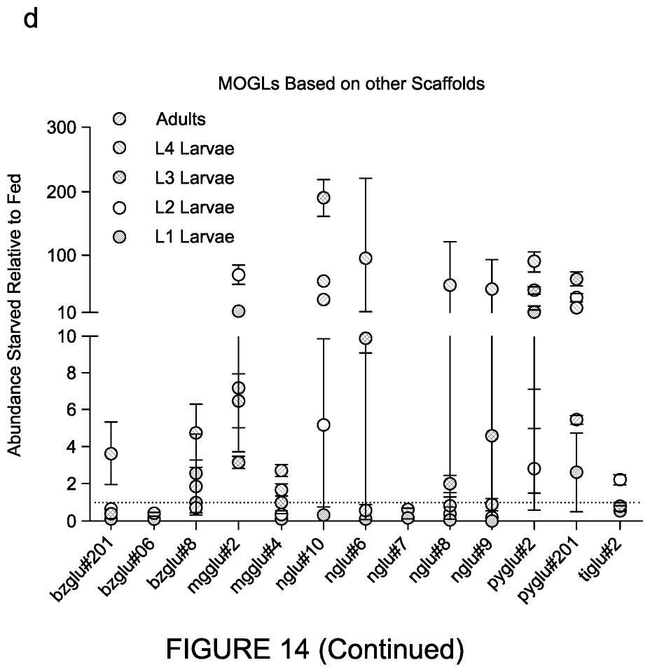

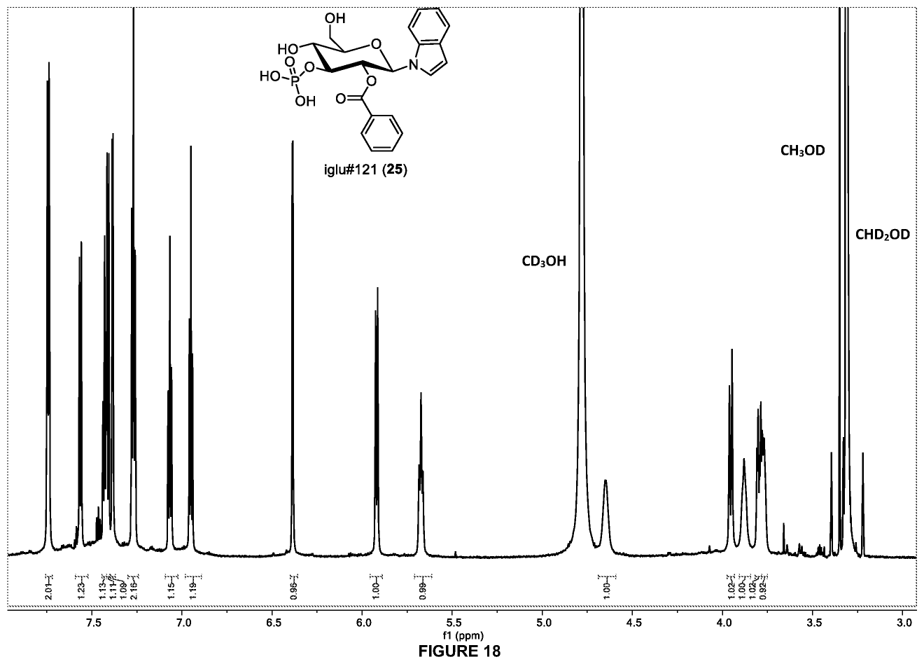

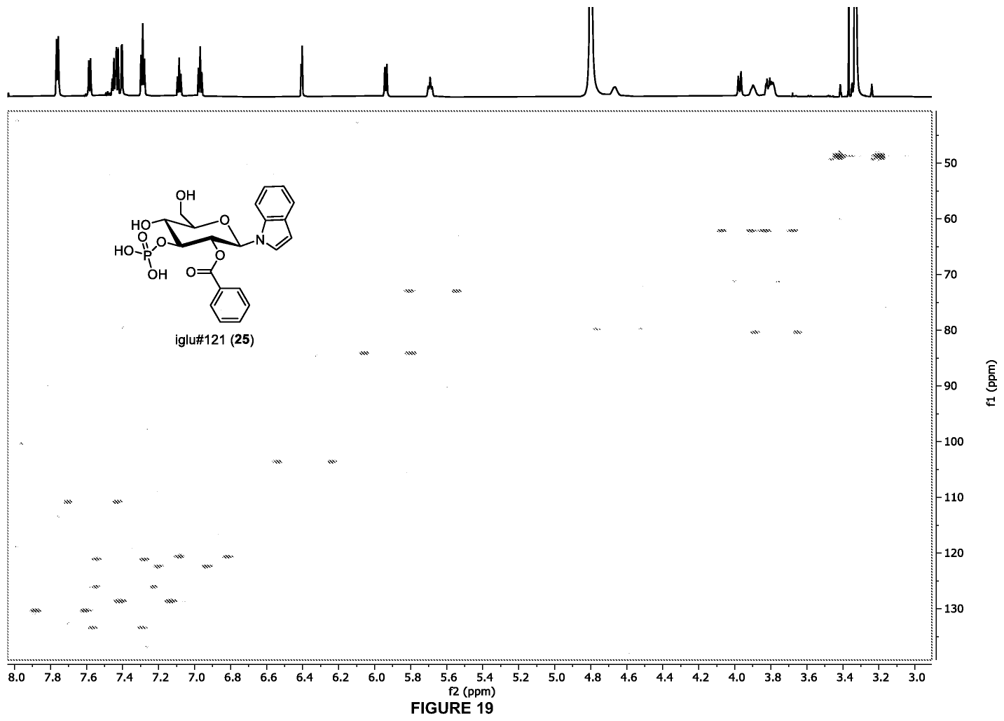

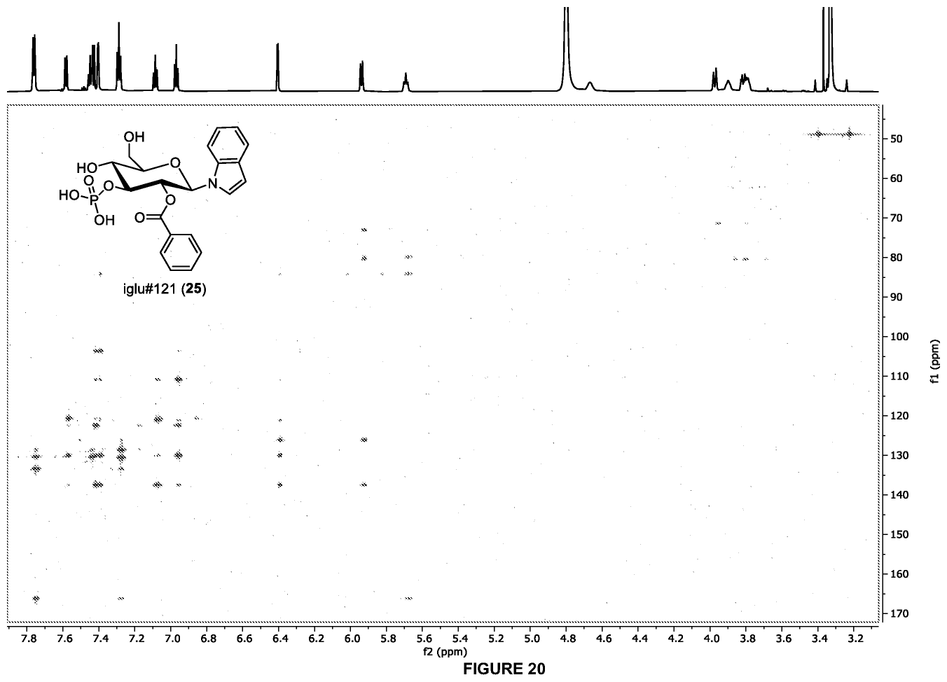

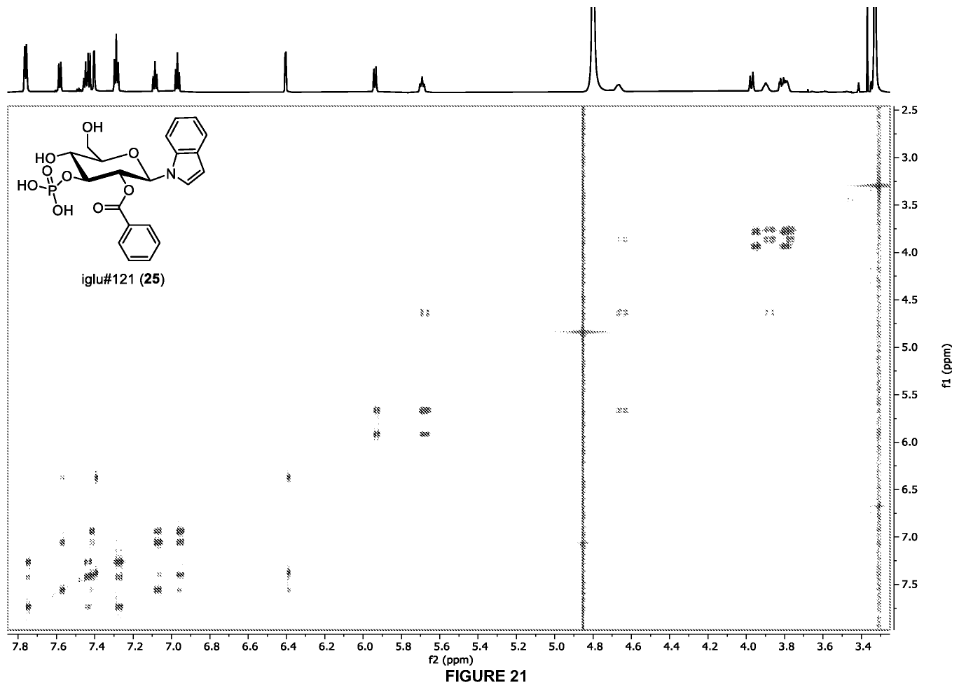

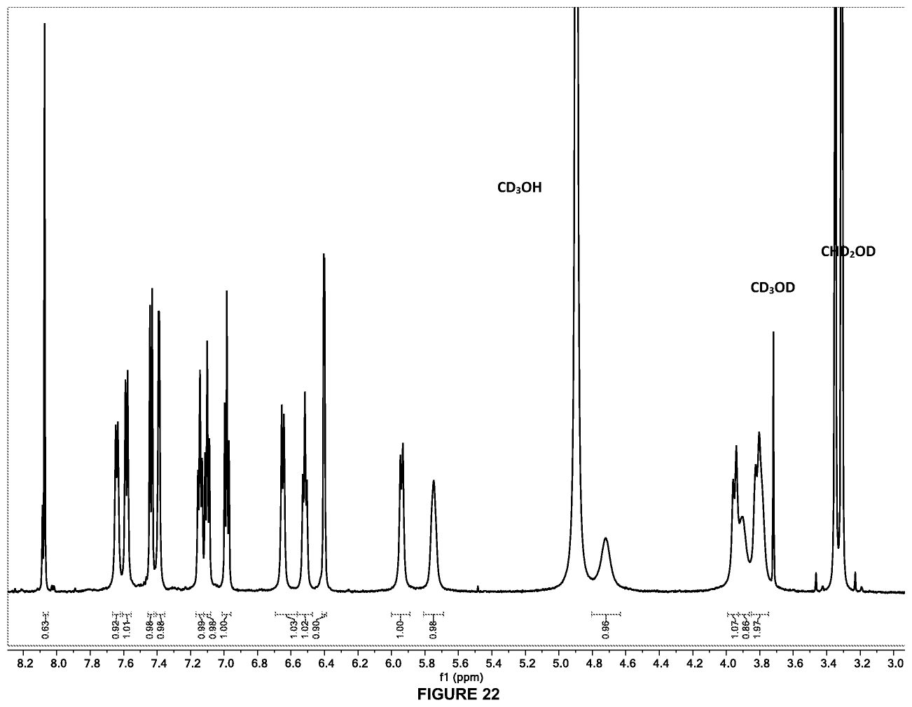

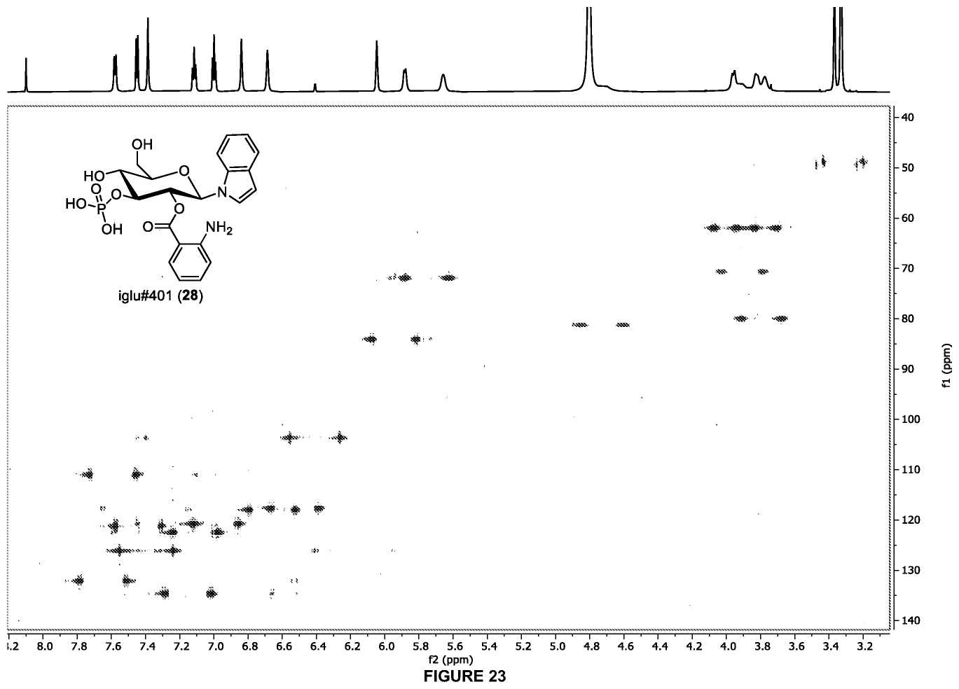

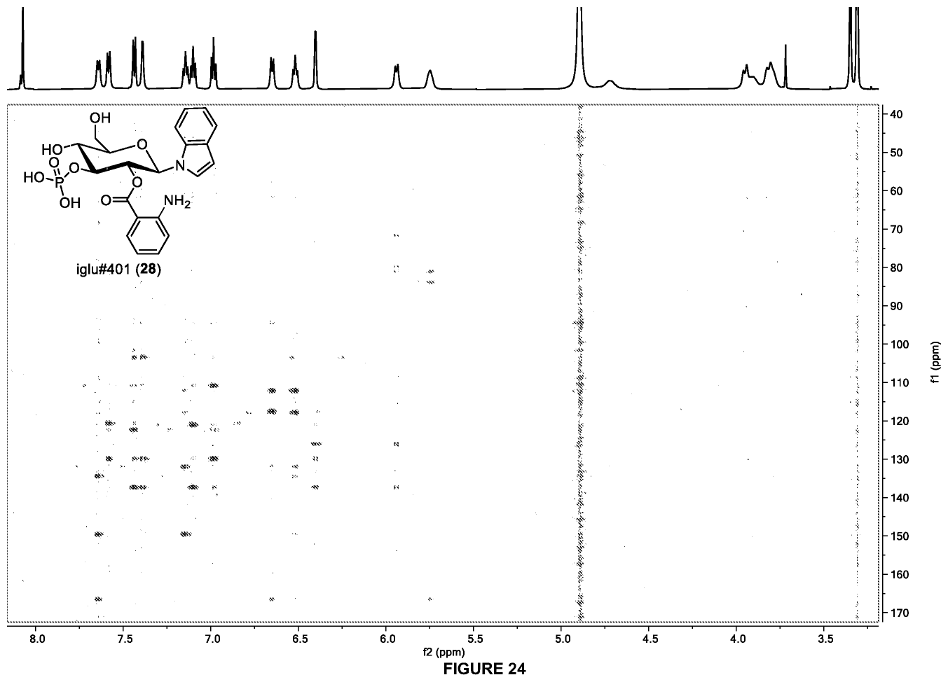

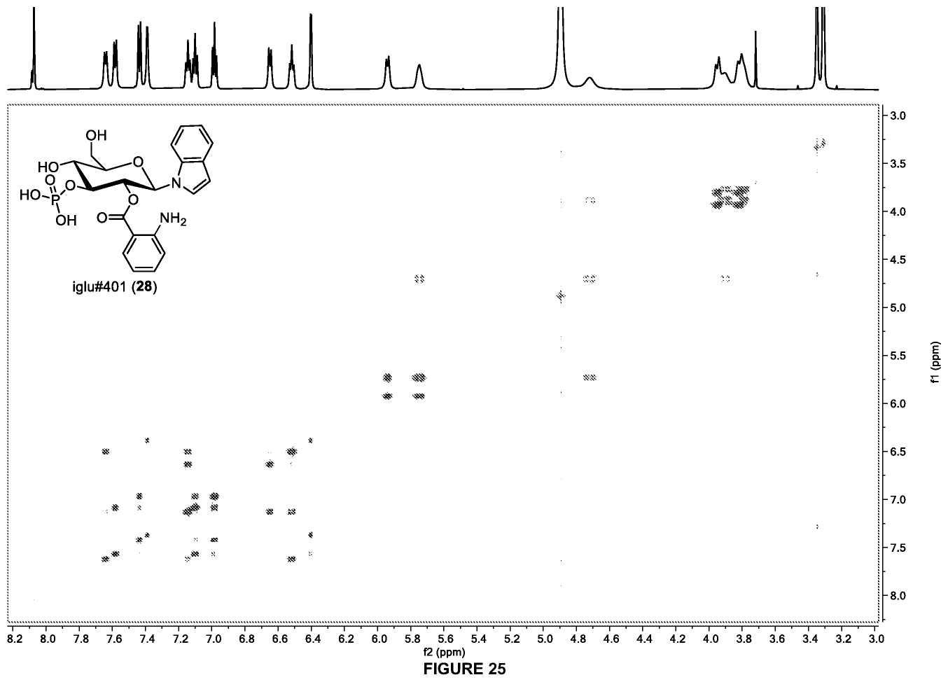

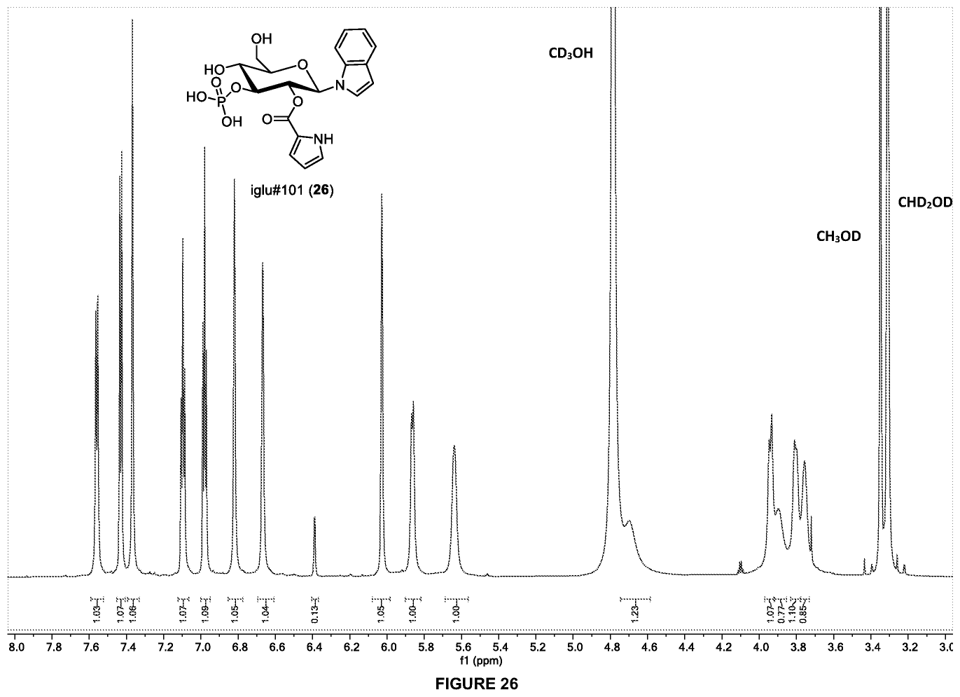

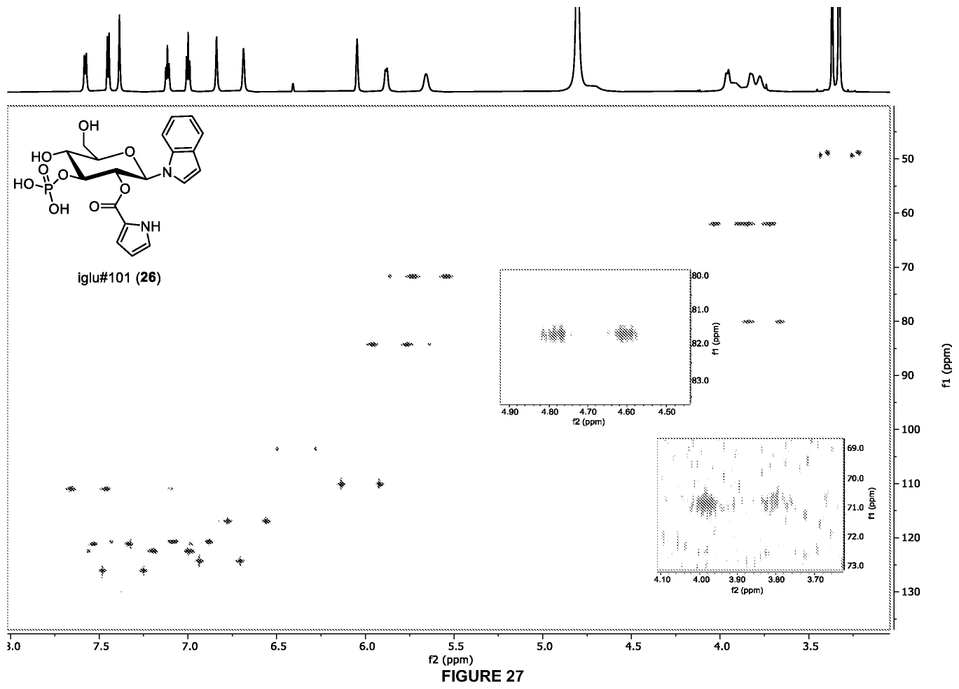

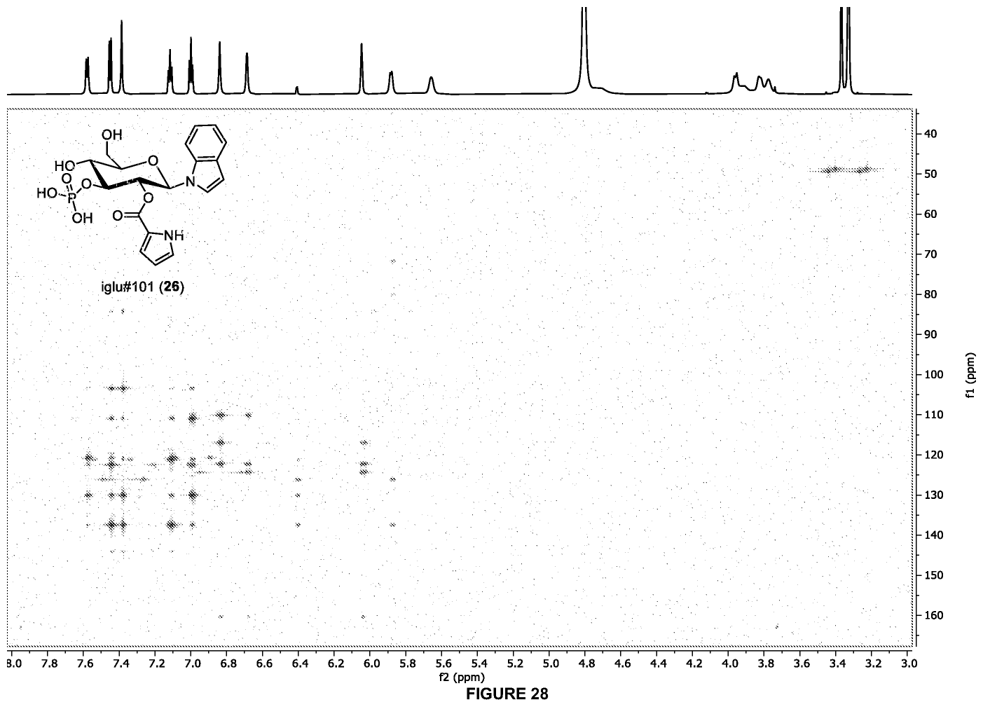

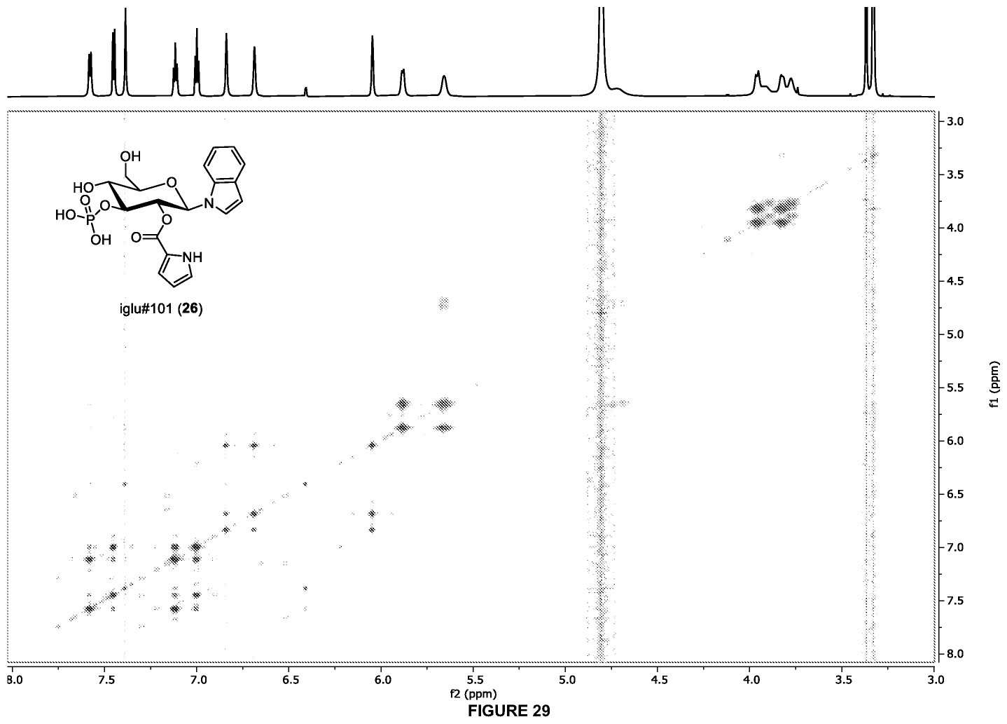

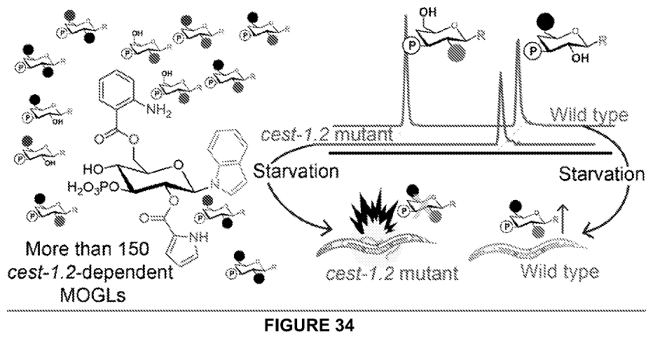

120. The compound of any one of the preceding embodiments, wherein the compound is a compound depicted in FIGS. 1-39, or a pharmaceutically acceptable salt thereof 121. The compound of any one of the preceding embodiments, wherein the compound is an isolated compound.

122. The compound of any one of the preceding embodiments, wherein the compound is a pure compound.

123. The compound of any one of the preceding embodiments, wherein the compound is provided outside of a C. elegans worm body.

124. The compound of any one of the preceding embodiments, wherein the compound is provided free of C. elegans tissue or other biological materials typically contained within or excreted by C. elegans.

125. A compound of any one of the preceding embodiments for use in medicine.

126. A therapeutic composition comprising a therapeutically effective amount of a compound of any one of the preceding embodiments.

127. A therapeutic composition for treating a disease or disorder, wherein the composition comprises one or more MOGLs of Formula I:

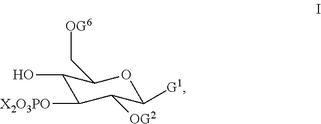

or a pharmaceutically acceptable salt thereof wherein:
G¹ is an optionally substituted moiety selected from the group consisting of: N-linked heterocycle (e.g., N-linked heteroaryl), —OR¹⁰ and —OC(O)R¹¹;
G² is an optionally substituted aliphatic, aromatic (e.g., aryl), heteroaromatic, or heteroaliphatic acyl group;
X is, independently at each occurrence, selected from the group consisting of —H, an optionally substituted phosphate or polyphosphate moiety, M⁺, and Z⁺;
M⁺ is any metal cation;
Z⁺ is an organic or inorganic ‘onium’ group comprising at least one nitrogen-, phosphorous-, or sulfur-based cation;
G⁶ is an optionally substituted aliphatic, aromatic, heteroaromatic, or heteroaliphatic acyl group; and
R¹⁰ and R¹¹ are each independently selected from the group consisting of: optionally substituted C₁₋₃₂ aliphatic, optionally substituted C₁₋₃₂ heteroaliphatic, optionally substituted aryl, and optionally substituted heteroaryl.

128. The therapeutic composition of embodiment 127, where the disease or disorder is a neurological disease,
and wherein the composition comprises one or more MOGLs selected from the group consisting of:

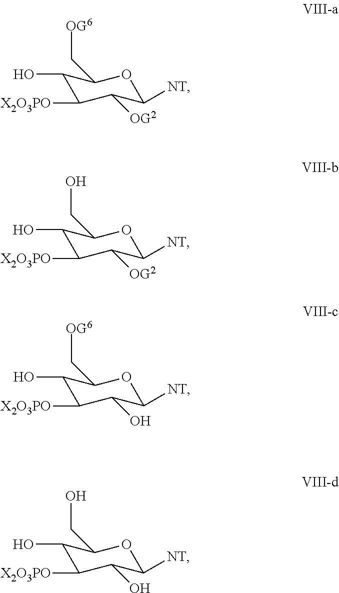

where the moiety —NT comprises a neurotransmitter, or a derivative or precursor of a neurotransmitter linked to the glucose through any suitable atom.

129. The therapeutic composition of embodiment 128, wherein the —NT comprises a monoamine neurotransmitter or a derivative or precursor thereof.

130. The therapeutic composition of embodiment 128, wherein the —NT is selected from the group consiting of: catecholamine neurotransmitters or derivatives or precursors thereof, dopamine, norepinepherine, epinepherine, histamine, serotonin, tryptamine, phenethylamine, N-methylphenethylamine, phenethanolamine, m-tyramine, p-tyramine, 3-methoxytyramine, N-methyltyramine, 3-indothyronamine, m-octopamine, p-octopamine, and synepherine.

131. The therapeutic composition of embodiment 127, where the disease or disorder is cancer, a kinase dependent disorder or disease such as hypertension, Parkinson's disease, and autoimmune disease, or a disorder that resulst in or arises from changes to nucleotide synthesis including, but not limited to cancer and viral diseases,
wherein the composition comprises one or more MOGLs selected from the group consisting of:

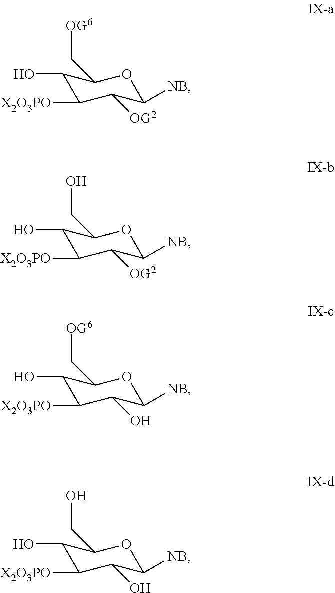

where —NB comprises an aromatic moiety, a nucleobase, or a derivative or precursor of a nucleobase linked to the glucose through any suitable atom.

132. The therapeutic composition of embodiment 131, wherein —NB comprises a nucleobase linked to the glucose through a nitrogen or oxygen atom comprising part of the nucleobase structure.

133. The therapeutic composition of embodiment 131, wherein —NB is selected from the group consisting of:

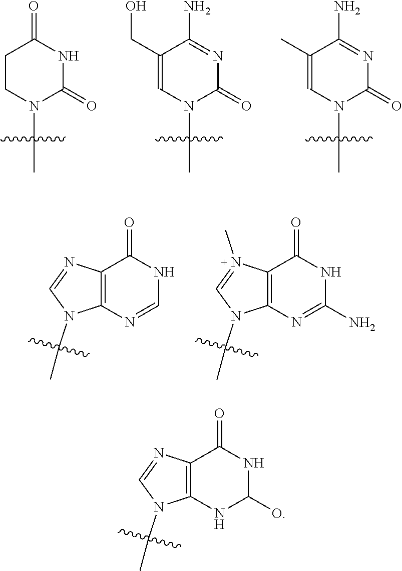

134. The therapeutic composition of embodiment 131, wherein —NB is selected from the group consisting of:

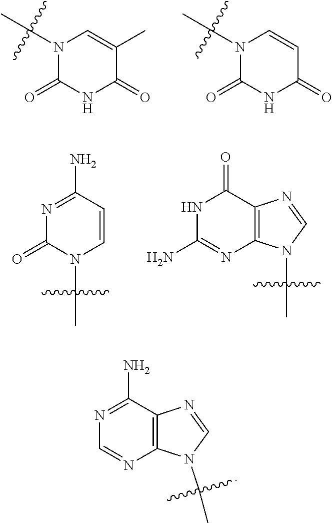

135. The therapeutic composition of embodiment 131, wherein —NB is selected from the group consisting of:

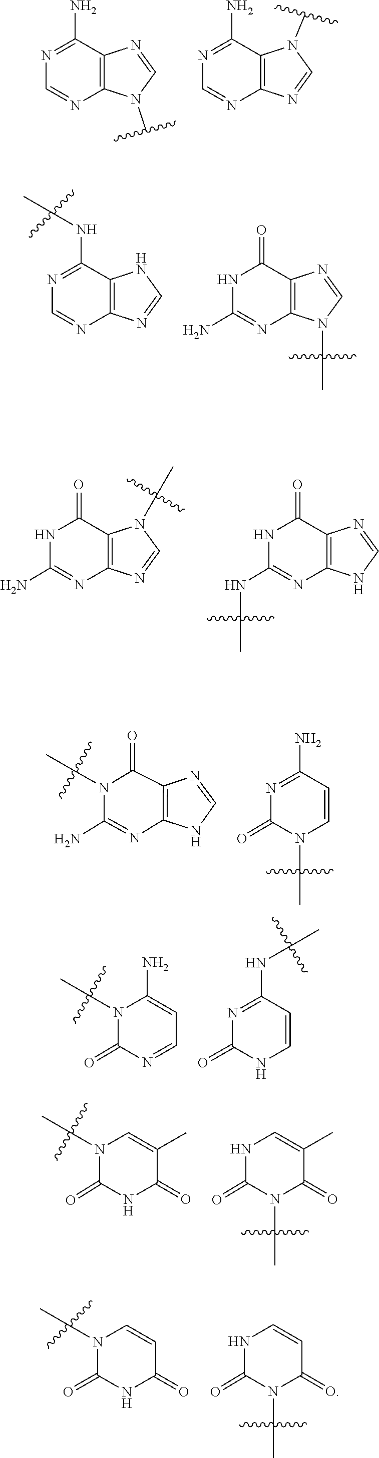

136. The therapeutic composition of embodiment 131, wherein —NB is selected from the group consisting of:

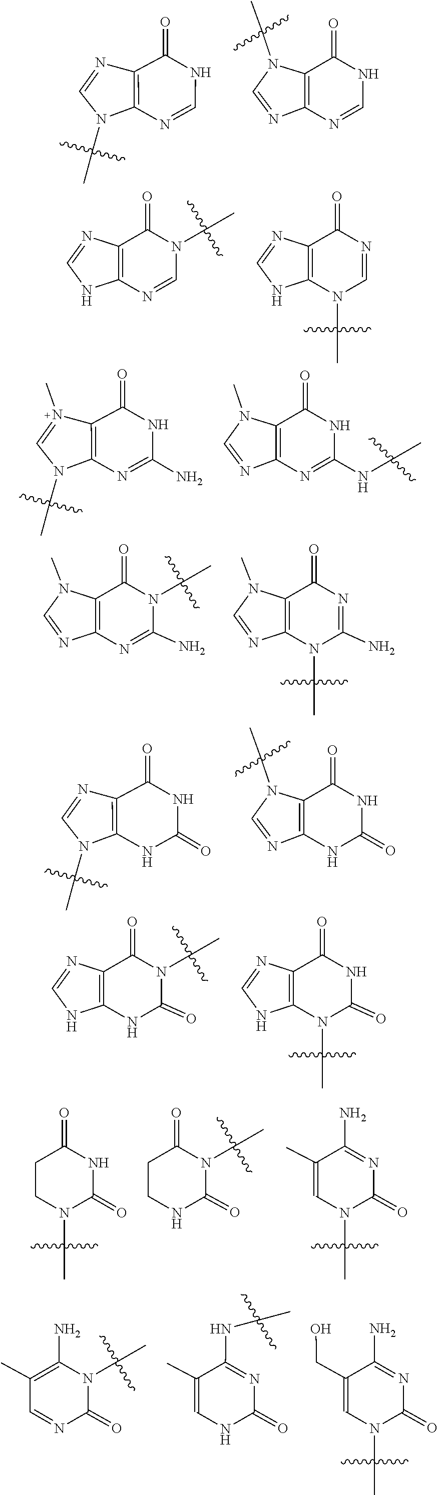

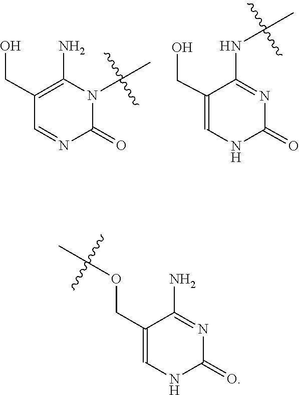 [SMILES: *n1c(N)c(CO)cnc1=O ; *Nc1nc(=O)[nH]cc1CO ; *OCc1c[nH]c(=O)nc1N]

137. The therapeutic composition of embodiment 127, where the disease or disorder is responsive to regulation of TOR function, and wherein the composition comprises one or more MOGLs selected from the group consisting of:

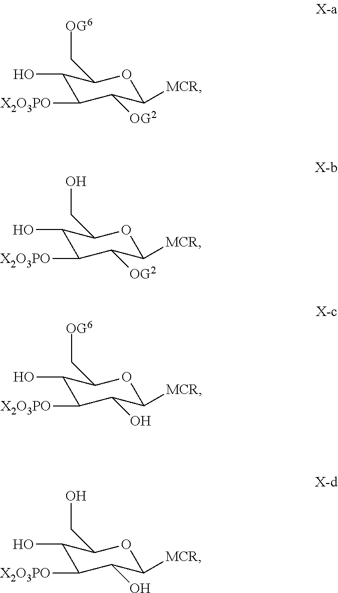

where -MCR—comprises a C₃₋₁₂ alpha beta unsaturated acyl group.

138. The therapeutic composition of embodiment 137, wherein -MCR comprises a C₃₋₈ alpha beta unsaturated acyl group, or wherein the moiety -MCR comprises a C₄₋₈ alpha beta unsaturated acyl group, or wherein the moiety -MCR comprises an acyl group corresponding to an ester of acrylic acid, methylacrylic acid, crotonic acid, methyl crotonic acid, valeric acid, 3-methylcrotonic acid, or tiglic acid.

139. The therapeutic composition of embodiment 137, wherein -MCR is selected from the group consisting of: crotonate, tiglate, valerate, acrylate, methacrylate, cinnamate, 2-imidazoleacrylate and urocanate.

140. A therapeutic composition for treatment of a disease or disorder responsive to regulation of proteasome function, wherein the composition comprises one or more MOGLs of Formula A-1:

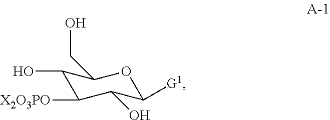

or a pharmaceutically acceptable salt thereof,
G¹ is an optionally substituted moiety selected from the group consisting of: N-linked heteroaryl, —OR¹⁰ and —OC(O)R¹¹;
X is, independently at each occurrence, selected from the group consisting of —H, an optionally substituted phosphate or polyphosphate moiety, M⁺, and Z⁺;
R¹⁰ and R¹¹ are each independently selected from the group consisting of: optionally substituted C₁₋₃₂ aliphatic, optionally substituted C₁₋₃₂ heteroaliphatic, optionally substituted aryl, and optionally substituted heteroaryl;
M⁺ is any metal cation; and
Z⁺ is an organic or inorganic ‘onium’ group comprising at least one nitrogen-, phosphorous-, or sulfur-based cation.

141. The compound or composition of any one of the preceding embodiments, wherein G² is an optionally substituted aliphatic acyl, optionally substituted aromatic acyl, optionally substituted heteroaromatic acyl, or optionally substituted heteroaliphatic acyl group.

142. The compound or composition of any one of the preceding embodiments, wherein G⁶ is an optionally substituted aliphatic acyl, optionally substituted aromatic acyl, optionally substituted heteroaromatic acyl, or optionally substituted heteroaliphatic acyl group.

143. The compound or composition of any one of the preceding embodiments, wherein each N-linked heterocycle is independently heteroaryl.

144. The compound or composition of any one of the preceding embodiments, wherein each aromatic is independently aryl (e.g., phenyl).

145. The compound or composition of any one of the preceding embodiments, wherein each heteroaliphatic is an independently an aliphatic group having 1-24 (e.g., 1-12, 1-8, or 1-6) carbons where 1-6 (e.g., 1-4, 1-3, or 1-2) carbons are independently replaced by a heteroatom selected from oxygen, sulfur, nitrogen, and phosphorus.

146. The compound or composition of any one of the preceding embodiments, wherein a heteroaryl ring is 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur.

147. The compound or composition of any one of the preceding embodiments, wherein a heterocylic ring is 3- to 7-membered saturated or partially unsaturated monocyclic heterocyclyl having 1-3 heteroatoms independently selected from nitrogen, oxygen, and sulfur, or 7- to 10-membered saturated or partially unsaturated bicyclic heterocyclyl having 1-4 heteroatoms independently selected from nitrogen, oxygen, and sulfur.

148. A pharmaceutical composition comprising a compound or composition of any one of the preceding embodiments and a pharmaceutically acceptable carrier or excipient.

149. A method of making a therapeutic composition comprising formulating an effective amount a compound or composition of any one of the preceding embodiments (or a pharmaceutically-acceptable salt, prodrug or derivative thereof) into a pharmaceutical composition selected from the group consisting of: injectible liquid, tablet, capsule, pill, solution or suspension for oral administration, solid for suspension or dissolution into a drinkable or injectible liquid, dermal patch, eye drop, cream, ointment, gel, powder, spray, and inhalable.

150. A method of making a therapeutic composition comprising formulating an effective amount of one or more purified or synthetically-produced MOGLs (or a pharmaceutically-acceptable salt, prodrug or derivative thereof) into a pharmaceutical composition selected from the group consisting of: injectible liquid, tablet, capsule, pill, solution or suspension for oral administration, solid for suspension or dissolution into a drinkable or injectible liquid, dermal patch, eye drop, cream, ointment, gel, powder, spray, and inhalable.

151. A method comprising administering to a mammal a therapeutically effective dose of one or more compounds of the preceding embodiments.

152. A method of improving the mental or emotional state of a patient comprising administering to a patient in need thereof a therapeutically effective amount of a compound of any one of the preceding embodiments.

153. A method of treating anxiety in a patient comprising administering to a patient in need thereof a therapeutically effective amount of a compound of any one of the preceding embodiments.

154. A method of treating depression in a patient comprising administering to a patient in need thereof a therapeutically effective amount of a compound of any one of the preceding embodiments.

155. A method of treating a neurological disorder in a patient comprising administering to a patient in need thereof a therapeutically effective amount of a compound of any one of the preceding embodiments.

156. The method of embodiment 155, wherein the neurological disorder is anxiety, depression, obsessive or compulsive disorders or behaviors, tics, bipolar disorder, schizophrenia, learning disorders, cognitive decline, behavioral disorders, learning disability, or hyperactivity.

157. A method of treating a kinase-dependent disease or disorder in a patient comprising administering to a patient in need thereof a therapeutically effective amount of a compound of any one of the preceding embodiments.

158. A method of treating diseases or disorders that result in or arise from changes to nucleotide synthesis comprising administering to a patient in need thereof a therapeutically effective amount of a compound of any one of the preceding embodiments.

159. The method of embodiment 158, wherein the disease or disorder is a cancer or a viral infection.

160. A method of treating a disease or disorder responsive to modulation of the proteasome, comprising administering to a patient in need thereof a therapeutically effective amount of a compound of any one of the preceding embodiments.

161. The method of embodiment 160, wherein the disease or disorder is cancer or neurodegenerative disease.

162. The method of embodiment 161, wherein the disease or disorder is Alzheimer's, Parkinson's, or Huntington's disease.

163. The method of any one of embodiments 160-162, wherein the compound is sngl #1, sngl #2, or a pharmaceutically acceptable salt thereof.

164. A method of treating or ameliorating a disease, disorder, or condition associated with a cellular or environmental stress response, comprising administering to a patient in need thereof a therapeutically effective amount of a compound of any one of the preceding embodiments.

165. The method of embodiment 164, wherein the stress response is oxidative stress response.

166. The method of embodiment 164 or 165, where the condition is shortened life span.

167. The method of embodiment 164 or 165, wherein the disease is cancer or a neurodegenerative disease.

168. The method of any one of embodiments 165-167, wherein the compound comprises an indole moiety at the 1-position (e.g., G¹).

169. A method treating a disease or disorder, comprising administering to a patient in need thereof a therapeutically effective amount of a composition comprising one or more MOGLs of Formula I:

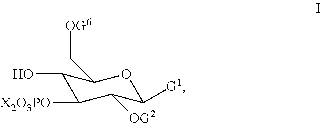

or a pharmaceutically acceptable salt thereof
wherein:
G¹ is an optionally substituted moiety selected from the group consisting of: N-linked heterocycle (e.g., N-linked heteroaryl), —OR¹⁰ and —OC(O)R¹¹;
G² is an optionally substituted aliphatic acyl, aromatic (e.g., aryl) acyl, heteroaromatic acyl, or heteroaliphatic acyl group;
X is, independently at each occurrence, selected from the group consisting of —H, an optionally substituted phosphate or polyphosphate moiety, M⁺, and Z⁺;
M⁺ is any metal cation;
Z⁺ is an organic or inorganic ‘onium’ group comprising at least one nitrogen-, phosphorous-, or sulfur-based cation;
G⁶ is an optionally substituted aliphatic acyl, aromatic (e.g., aryl) acyl, heteroaromatic acyl, or heteroaliphatic acyl group; and
R¹⁰ and R¹¹ are each independently selected from the group consisting of optionally substituted C₁₋₃₂ aliphatic, optionally substituted C₁₋₃₂ heteroaliphatic, optionally substituted aryl, and optionally substituted heteroaryl.

170. The method of embodiment 169, where the disease or disorder is a neurological disease, and wherein the composition comprises one or more MOGLs selected from the group consisting of:

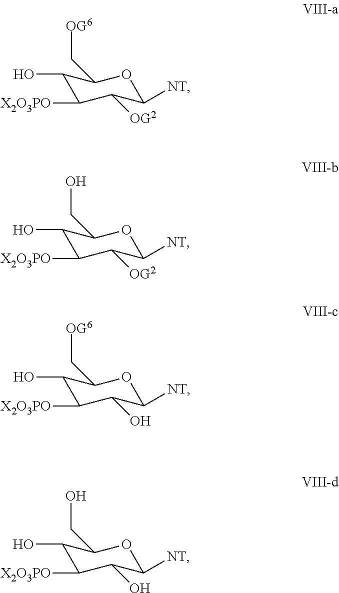

where the moiety —NT comprises a neurotransmitter, or a derivative or precursor of a neurotransmitter linked to the glucose through any suitable atom.

171. The method of embodiment 170, wherein the —NT comprises a monoamine neurotransmitter or a derivative or precursor thereof.

172. The method of embodiment 170, wherein the —NT is selected from the group consiting of catecholamine neurotransmitters or derivatives or precursors thereof, dopamine, norepinepherine, epinepherine, histamine, serotonin, tryptamine, phenethylamine, N-methylphenethylamine, phenethanolamine, m-tyramine, p-tyramine, 3-methoxytyramine, N-methyltyramine, 3-indothyronamine, m-octopamine, p-octopamine, and synepherine.

173. The method of embodiment 169, where the disease or disorder is cancer, a kinase dependent disorder or disease such as hypertension, Parkinson's disease, and autoimmune disease, or a disorder that resulst in or arises from changes to nucleotide synthesis including, but not limited to cancer and viral diseases,
wherein the composition comprises one or more MOGLs selected from the group consisting of

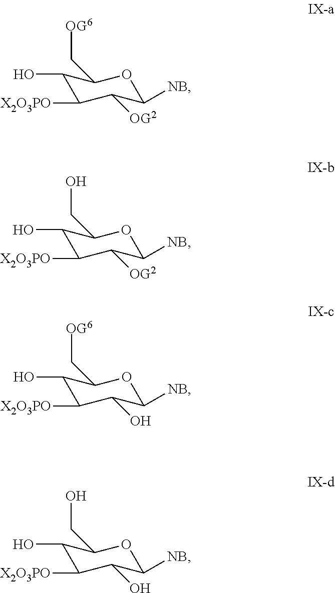

where —NB comprises an aromatic moiety, a nucleobase, or a derivative or precursor of a nucleobase linked to the glucose through any suitable atom.

174. The method of embodiment 173, wherein —NB comprises a nucleobase linked to the glucose through a nitrogen or oxygen atom comprising part of the nucleobase structure.

175. The method of embodiment 173, wherein —NB is selected from the group consisting of:

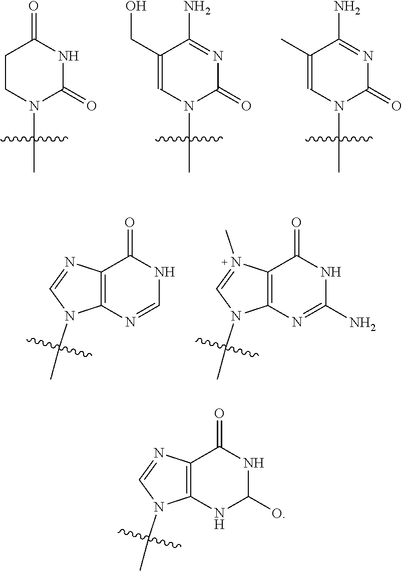

176. The method of embodiment 173, wherein —NB is selected from the group consisting of:

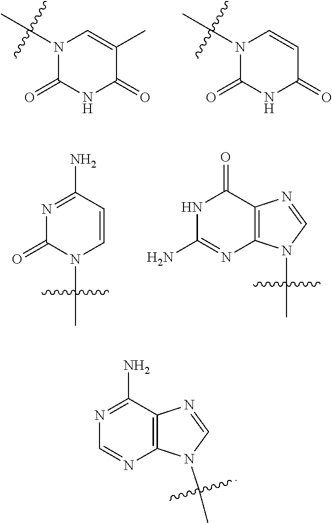

177. The method of embodiment 173, wherein —NB is selected from the group consisting of:

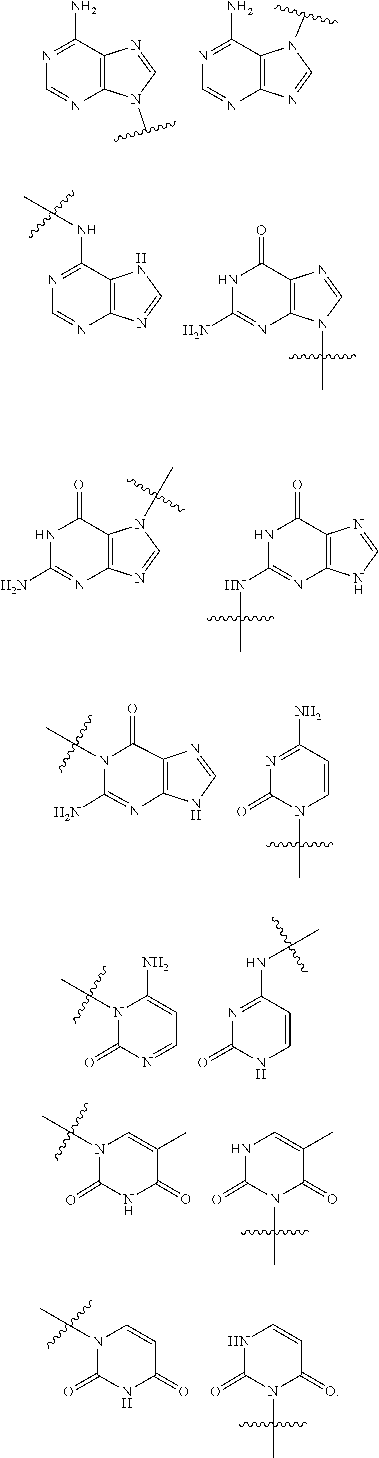

178. The method of embodiment 173, wherein —NB is selected from the group consisting of:

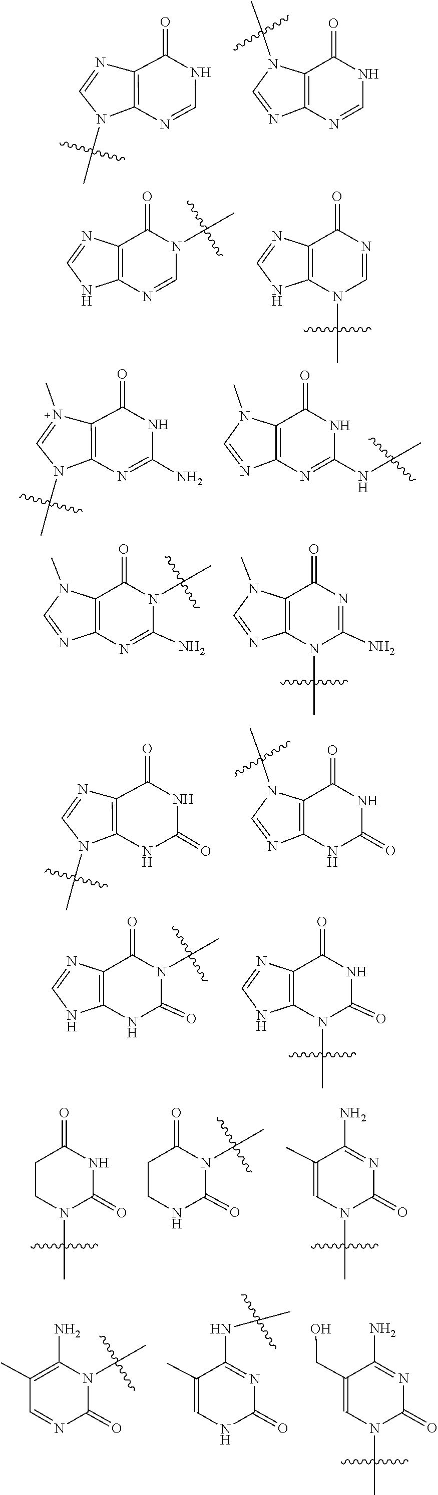

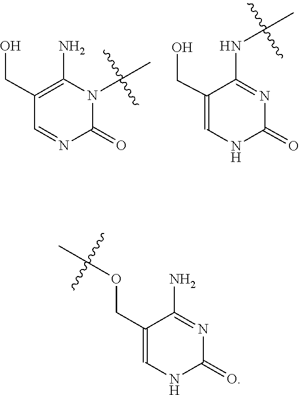 [SMILES: *n1c(N)c(CO)cnc1=O ; *Nc1nc(=O)[nH]cc1CO ; *OCc1c[nH]c(=O)nc1N]

179. The method of claim 169, where the disease or disorder is responsive to regulation of TOR function, and wherein the composition comprises one or more MOGLs selected from the group consisting of:

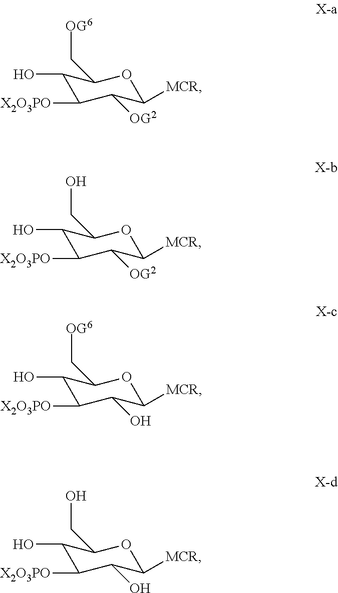

where -MCR—comprises a C₃₋₁₂ alpha beta unsaturated acyl group.

180. The method of claim 179, wherein -MCR comprises a C₃₋₈ alpha beta unsaturated acyl group, or wherein the moiety -MCR comprises a C₄₋₈ aloha beta unsaturated acyl group, or wherein the moiety -MCR comprises an acyl group corresponding to an ester of acrylic acid, methylacrylic acid, crotonic acid, methyl crotonic acid, valeric acid, 3-methylcrotonic acid, or tiglic acid.

181. The method of claim 179, wherein -MCR is selected from the group consisting of crotonate, tiglate, valerate, acrylate, methacrylate, cinnamate, 2-imidazoleacrylate and urocanate.

182. A method for treating a disease or disorder responsive to regulation of proteasome function, comprising administering to a patient in need thereof a therapeutically effective amount of a composition comprising one or more MOGLs of Formula A-1:

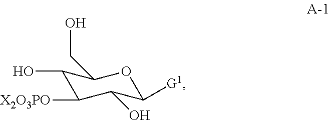

or a pharmaceutically acceptable salt thereof,
G¹ is an optionally substituted moiety selected from the group consisting of: N-linked heteroaryl, —OR¹⁰ and —OC(O)R¹¹;
X is, independently at each occurrence, selected from the group consisting of —H, an optionally substituted phosphate or polyphosphate moiety, M⁺, and Z⁺;
R¹⁰ and R¹¹ are each independently selected from the group consisting of optionally substituted C₁₋₃₂ aliphatic, optionally substituted C₁₋₃₂ heteroaliphatic, optionally substituted aryl, and optionally substituted heteroaryl;
M⁺ is any metal cation; and
Z⁺ is an organic or inorganic ‘onium’ group comprising at least one nitrogen-, phosphorous-, or sulfur-based cation.

183. A method comprising administering to a mammal a composition comprising a therapeutically effective amount of one or more MOGLs of Formula I:

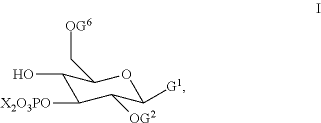

or a pharmaceutically acceptable salt thereof
wherein:
G¹ is an optionally substituted moiety selected from the group consisting of: N-linked heterocycle (e.g., N-linked heteroaryl), —OR¹⁰ and —OC(O)R¹¹;
G² is an optionally substituted aliphatic, aromatic (e.g., aryl), heteroaromatic, or heteroaliphatic acyl group;
X is, independently at each occurrence, selected from the group consisting of —H, an optionally substituted phosphate or polyphosphate moiety, M⁺, and Z⁺;
G⁶ is an optionally substituted aliphatic, aromatic (e.g., aryl), heteroaromatic, or heteroaliphatic acyl group; and
wherein, R¹⁰ and R¹¹ are each independently selected from the group consisting of: optionally substituted C₁₋₃₂ aliphatic, optionally substituted C₁₋₃₂ heteroaliphatic, optionally substituted aryl, and optionally substituted heteroaryl.

184. The method of claim 183, where the disease or disorder is cancer or another other kinase dependent disorder or disease such as hypertension, Parkinson's disease, and autoimmune disease, or a disorder that results in or arises from changes to nucleotide synthesis including cancer and viral diseases,
wherein the one or more MOGLs is selected from the group consisting of:

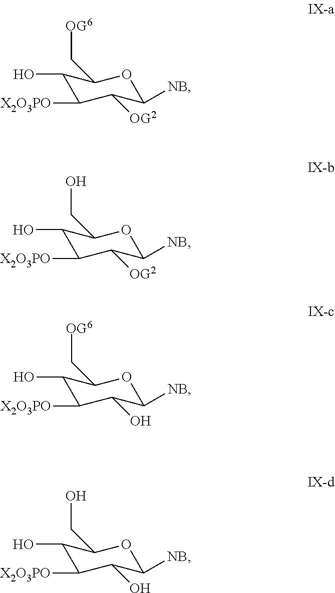

where —NB comprises an aromatic moiety, a nucleobase, or a derivative or precursor of a nucleobase linked to the glucose through any suitable atom.

185. The method of claim 183, where the disease or disorder is a neurological disease, and wherein the one or more MOGLs selected from the group consisting of:

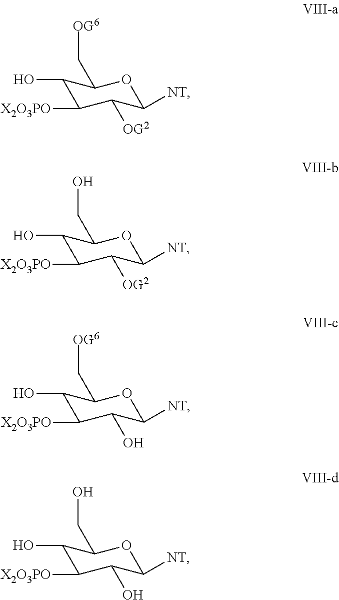

where the moiety —NT comprises a neurotransmitter, or a derivative or precursor of a neurotransmitter linked to the glucose through any suitable atom.

186. The method of claim 183, where the disease or disorder is one responsive to regulation of TOR function, and wherein the composition comprises one or more MOGLs selected from the group consisting of:

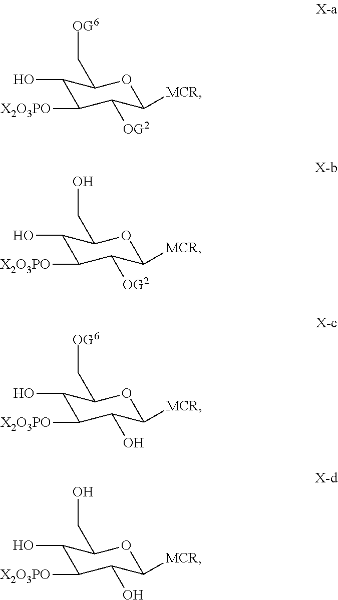

where -MCR—comprises a C₃₋₁₂ alpha beta unsaturated acyl group.

187. A method comprising administering to a mammal a composition comprising a therapeutically effective amount of one or more MOGLs of Formula A-1 or A-2:

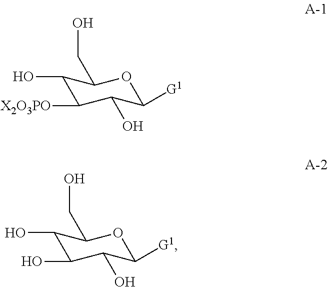

or a pharmaceutically acceptable salt thereof,
G¹ is an optionally substituted moiety selected from the group consisting of: N-linked heteroaryl, —OR¹⁰ and —OC(O)R¹¹;
X is, independently at each occurrence, selected from the group consisting of —H, an optionally substituted phosphate or polyphosphate moiety, M⁺, and Z⁺;
R¹⁰ and R¹¹ are each independently selected from the group consisting of: optionally substituted C₁₋₃₂ aliphatic, optionally substituted C₁₋₃₂ heteroaliphatic, optionally substituted aryl, and optionally substituted heteroaryl;
M⁺ is any metal cation; and
Z⁺ is an organic or inorganic ‘onium’ group comprising at least one nitrogen-, phosphorous-, or sulfur-based cation.

188. The method of any one of the preceding claims, wherein the composition is a pharmaceutical composition comprising a pharmaceutically acceptable excipient or carrier.

### EXAMPLES

The following Examples are useful to confirm aspects of the disclosure described above and to exemplify certain embodiments of the disclosure.

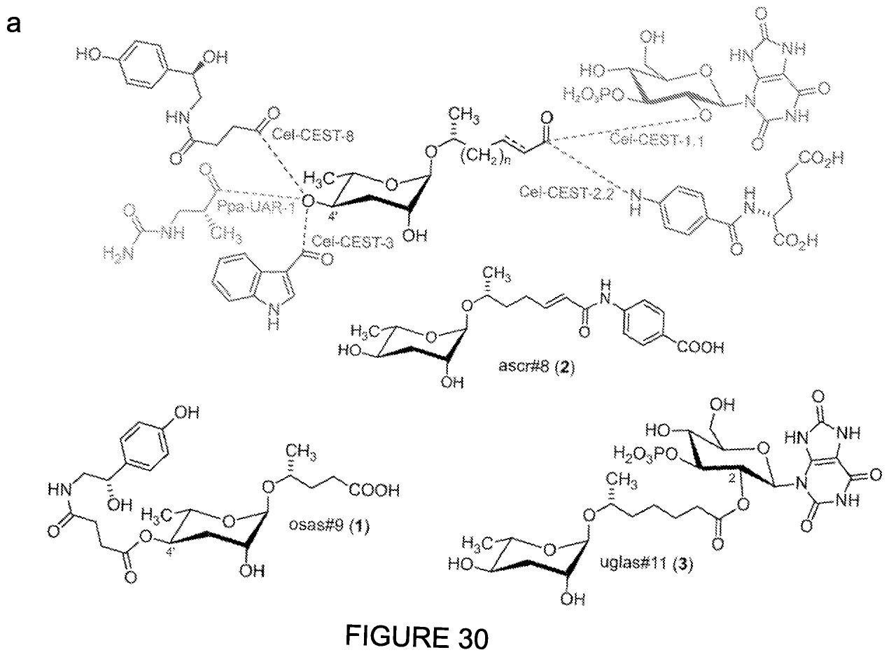

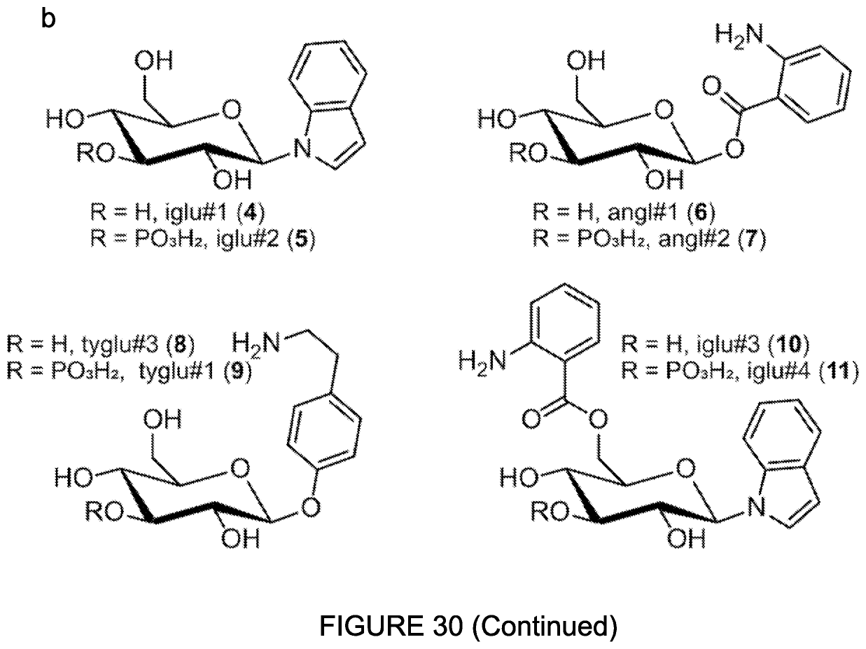

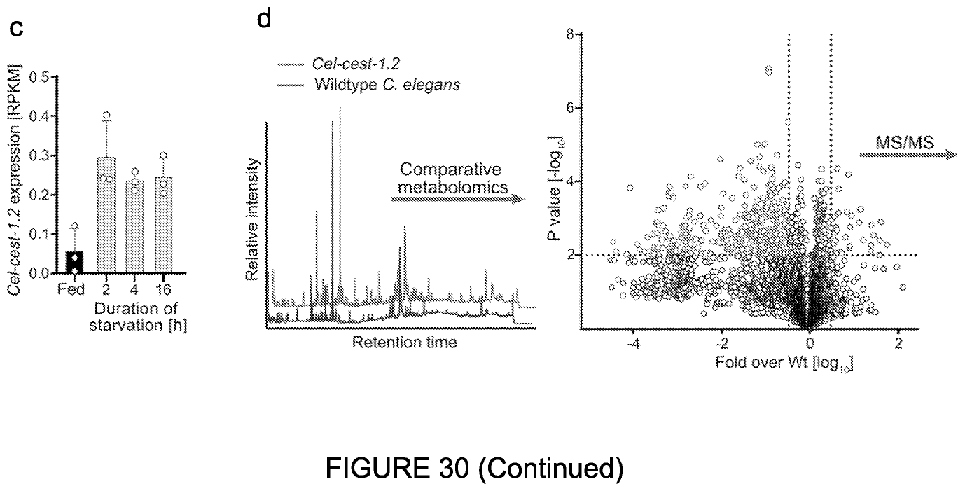

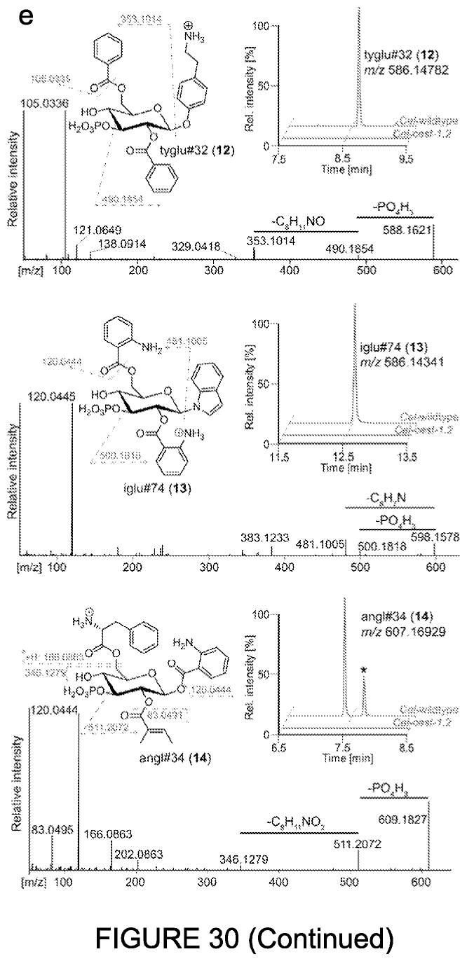

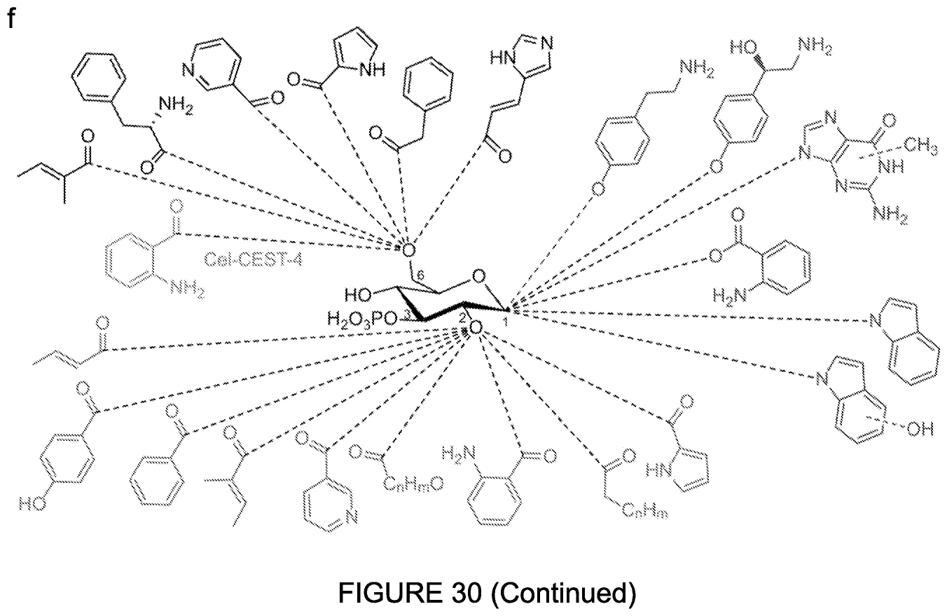

Cel-CEST-1.2 contributes to biosynthesis of >150 MOGLs. Following the initial discovery of certain MOGLs,^{7,14,16} we noted that their production is greatly increased under starvation conditions. Surveying published transcriptomic datasets for starvation-induced cest-homologs, we noted that Cel-cest-1.2 expression is rapidly induced 4-5-fold by starvation (FIG. 30c).¹⁵ Cel-cest-1.2 is a close paralog of Cel-cest-1.1, which we had recently shown to be required for attachment of the ascaroside side chain to the 2-position of the gluconucleoside moiety in uglas #11 (3) (FIG. 30a). Therefore, we hypothesized that Cel-cest-1.2, may be required for the production of 2-O-acylated MOGLs. Like Cel-CEST-1.1, Cel-CEST-1.2 features a conserved C-terminal transmembrane domain and is predicted to be expressed primarily in the intestine (FIG. 1).¹⁷

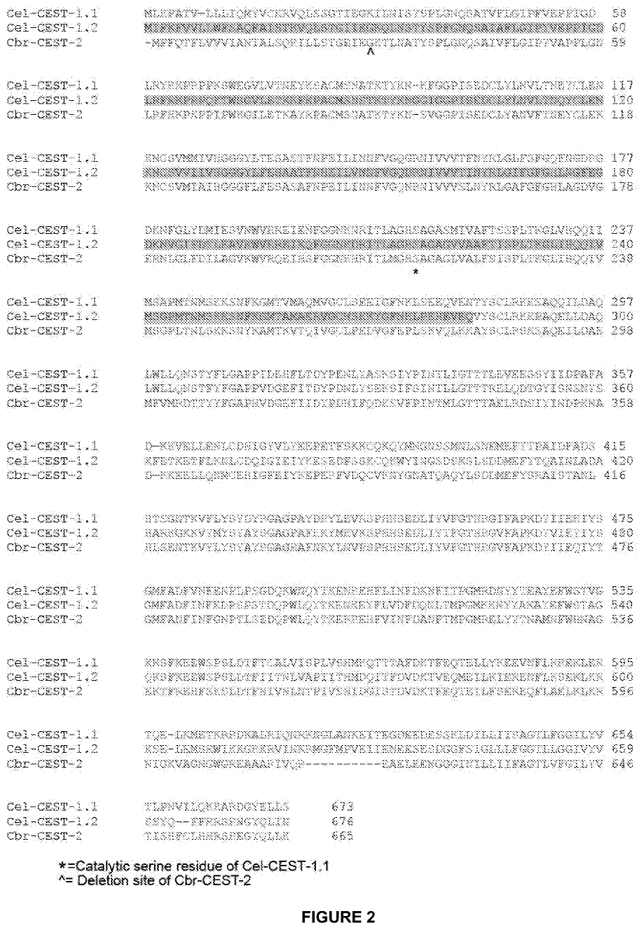

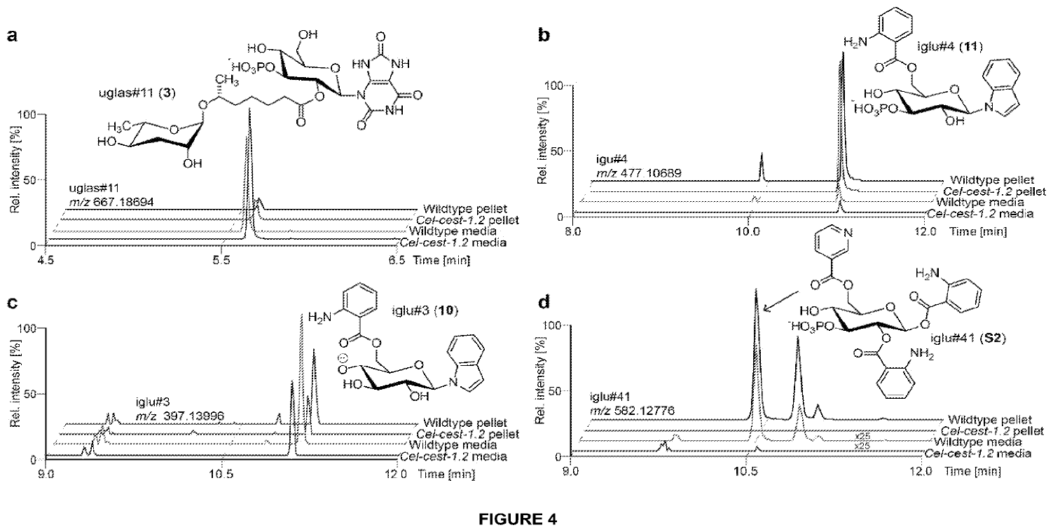

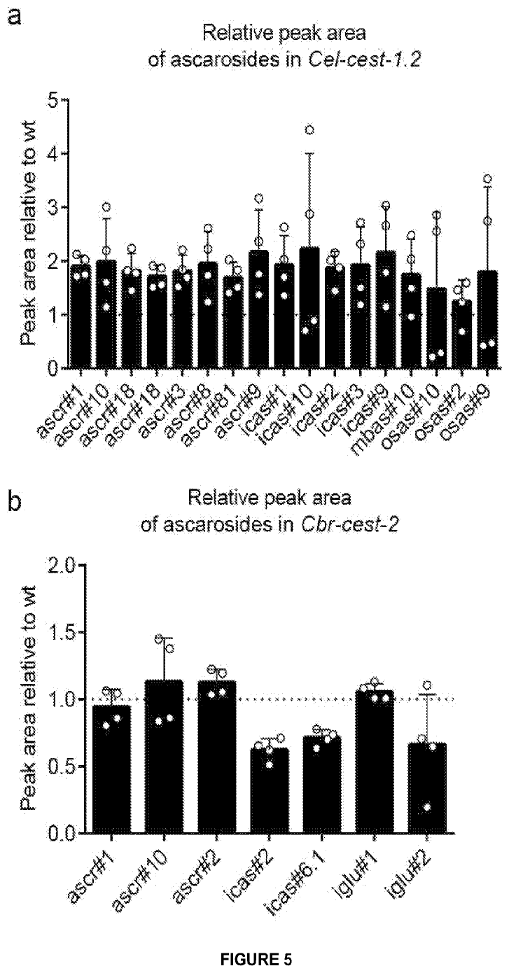

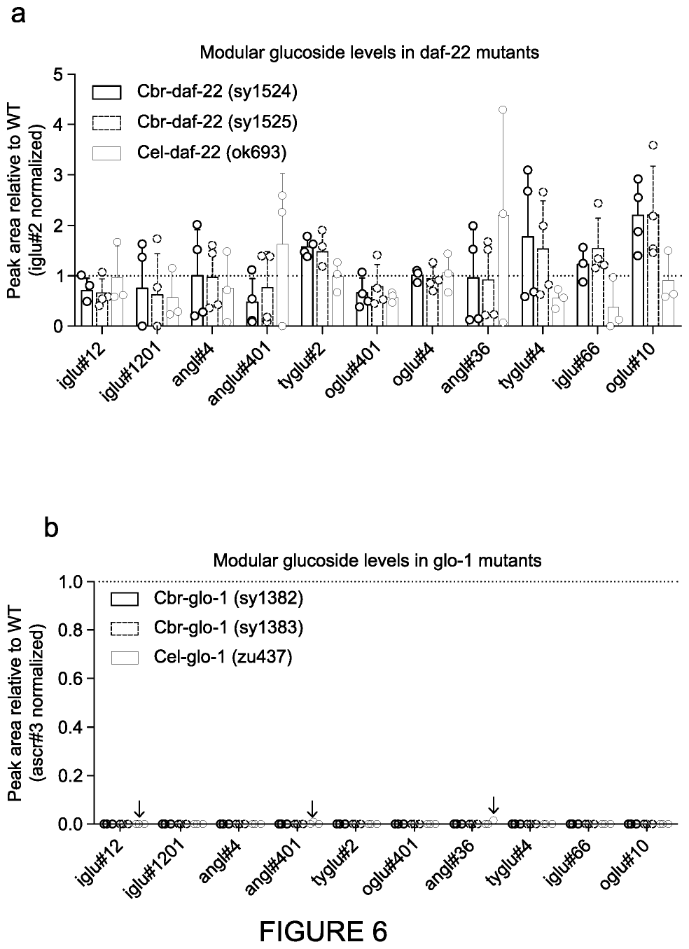

To investigate the biosynthetic role of Cel-cest-1.2, we obtained a mutant lacking the first 1500 bp of the coding sequence, including the serine at the putative active site (FIG. 2). Using HPLC-HRMS followed by comparative analysis utilizing the Metaboseek platform, we analyzed the endo-metabolome (compounds extractable from the worm bodies) and exo-metabolome (compounds secreted into the media) of Cel-cest-1.2 mutants for compounds whose production was more than 50-fold reduced compared to C. elegans wildtype (FIG. 30d, Table S4).¹⁸ These analyses revealed that Cel-cest-1.2 deletion has a dramatic impact on the C. elegans metabolome, as we detected >150 distinct metabolites whose production were strongly reduced or abolished in Cel-cest-1.2 mutants (FIG. 30d, Table S4). Most of the Cel-cest-1.2-dependent compounds were detected in the endo-metabolome, whereas comparatively few differences were observed in the exo-metabolomes. MS/MS fragmentation indicated that most of the detected Cel-cest-1.2-dependent metabolites are based on the recently described MOGL scaffolds and are further modified with a wide variety of acyl moieties, primarily derived from amino acid and fatty acid metabolism (FIGS. 30e, f, 3, Table S4). In contrast, production of the metabolites previously shown to be Cel-cest-1.1-dependent (e.g. uglas #11, 3) or Cel-cest-4 dependent (e.g. iglu #4, 11) was not affected in the Cel-cest-1.2 mutant (FIG. 4). Similarly, abundances of ascarosides were largely unchanged in Cel-cest-1.2 mutants (FIG. 5). Conversely, none of the Cel-cest-1.2-dependent compounds were abolished in mutants of Cel-daf-22, which codes for a peroxisomal 3-ketoacylthiolase required for ascaroside biosynthesis (FIG. 6a).^{19,20} consistent with previous results for the role of LROs in MOGL biosynthesis, production of Cel-cest-1.2-dependent compounds was also strongly reduced or abolished in LRO-defective Cel-glo-1 mutants (FIG. 6b).

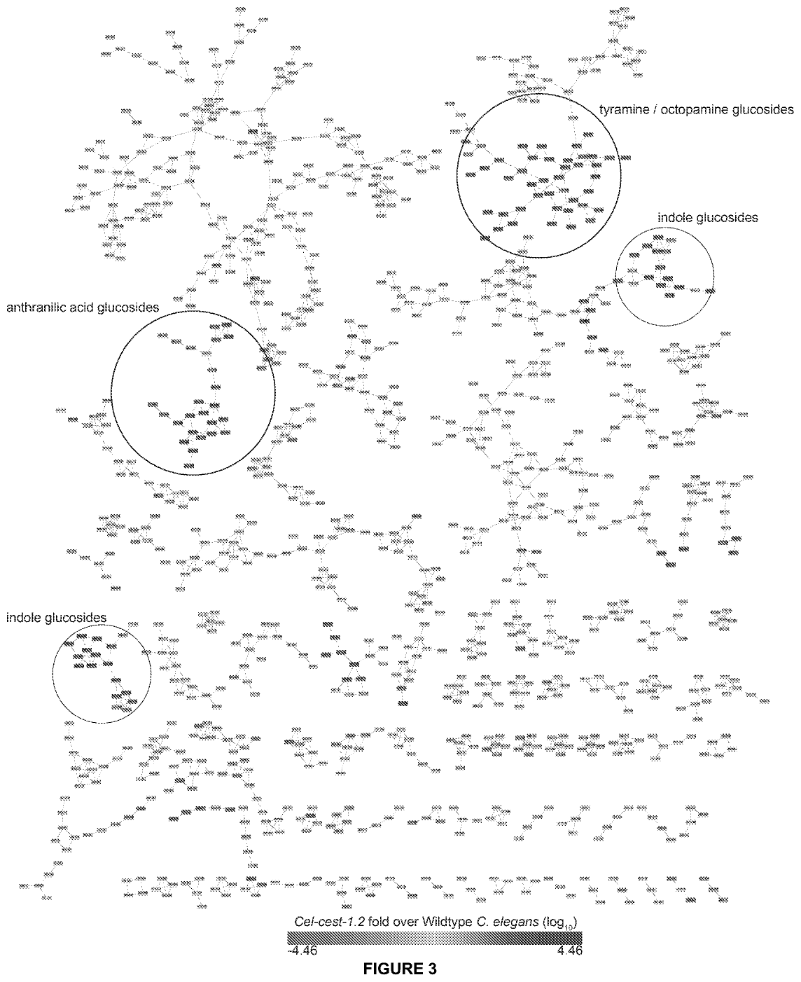

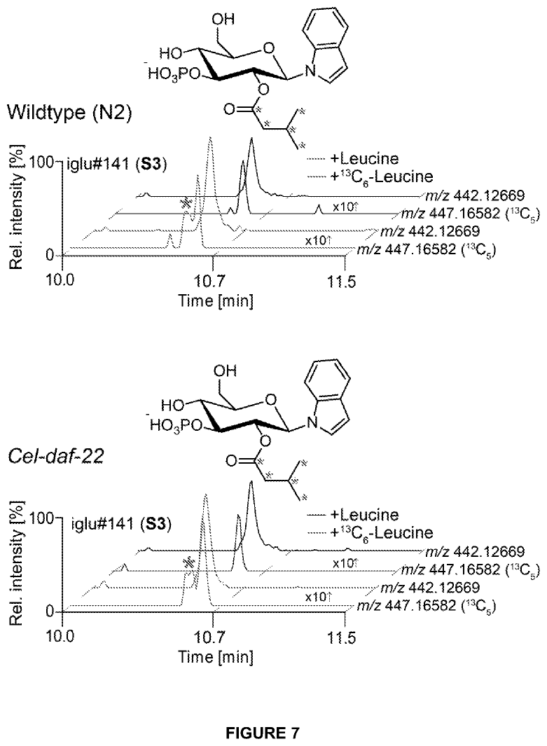

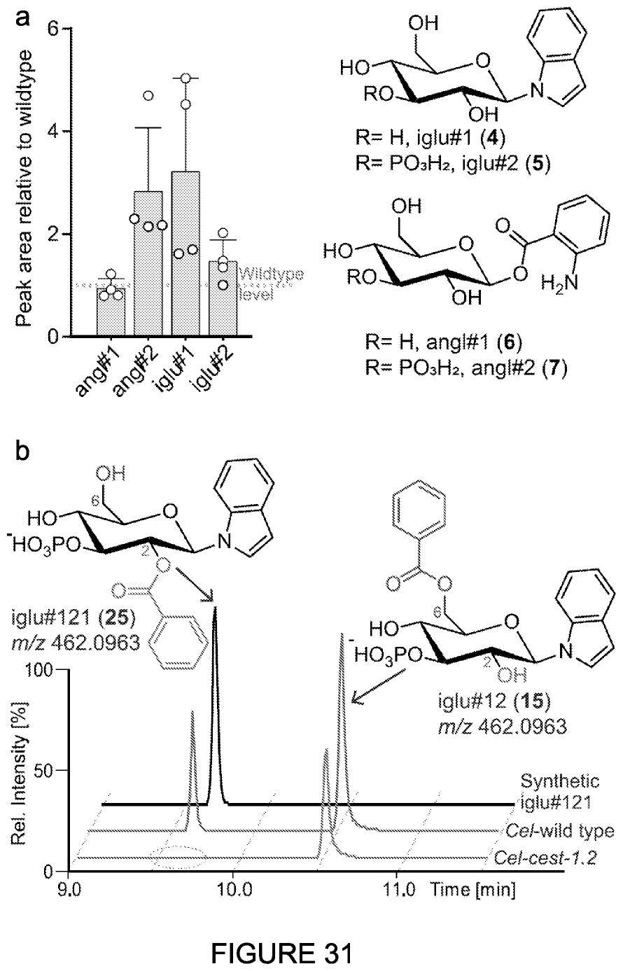

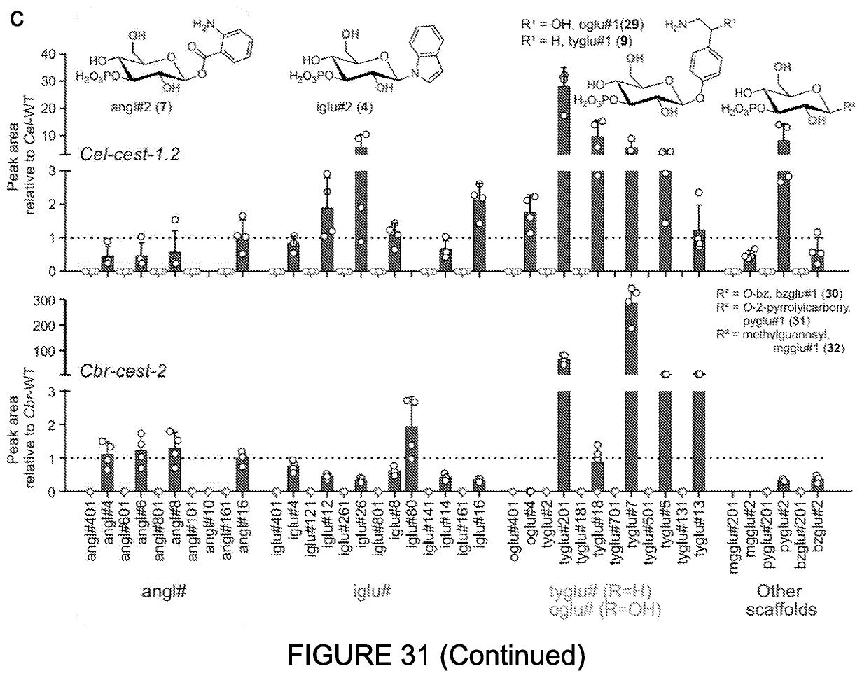

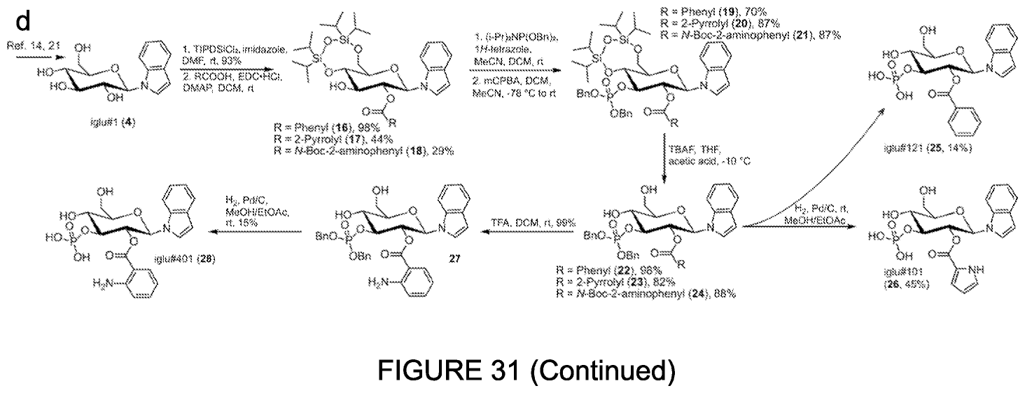

Next, we categorized the large number of Cel-cest-1.2-dependent metabolites based on their MS/MS fragmentation patterns, which enabled putative assignment to families of MOGLs based on several different scaffolds, e.g. N- or O-glucosylated indole and anthranilic acid (FIGS. 30e and 3, Table S4). Importantly, biosynthesis of the unmodified parent scaffolds, e.g. iglu #1 (4) or angl #2 (7), is not abolished in Cel-cest-1.2 mutants (FIG. 31a). Instead, abundances of these parent scaffolds are slightly increased relative to wildtype C. elegans, suggesting that they may accumulate as shunt metabolites. Detailed analysis of the MS/MS fragmentation patterns further suggested that all Cel-cest-1.2-dependent metabolites are derived from attachment of one or two of 16 different acyl moieties to the parent scaffolds (FIG. 30f, Table S4), some of which we had previously shown to be incorporated into MOGLs.⁷ Metabolomic analysis of wildtype C. elegans supplemented with isotope labeled L-[U-¹³C₆]-leucine and L-[3,3-D₂]-tyrosine supported the assignment of isovaleryl as well as tyramine and octopamine moieties in the identified MOGLs (FIG. 7, Table S4).^{7,22} CEST-1.2 is specifically required for 2-O-acylation. Based on the previous examples, we proposed that Cel-cest-1.2-dependent MOGLs are 3-O-phosphorylated and feature 2-O— and/or 6-O acylation (FIG. 30e, S4d).^{7,14} Importantly, almost all mono-acylated MOGLs were represented by two isomers with near-identical MS/MS fragmentation patterns but distinct HPLC retention times. Of these, only the earlier eluting isomer was abolished in Cel-cest-1.2 mutants, whereas abundance of the later eluting isomers was generally unchanged or increased (FIGS. 31b, c, 8).

These results suggested that Cel-CEST-1.2 may be required for site-selective acylation of the parent glucoside scaffolds. To determine whether Cel-CEST-1.2 is responsible for 2- or 6-O-acylation, we selected the 2-O-acylated variants of three mono-acylated MOGLs for total synthesis via established methods (FIG. 31d).^{7,23} To selectively synthesize 2-O-acylated MOGLs, scaffold iglu #1 (4), was 4,6-di-O-protected using 1,3-dichloro-1,1,3,3-tetraisopropyldi-siloxane. Esterification with different carboxylic acids gratuitously yielded primarily the 2-O-acylated derivative, which was 3-O-phosphorylated and subsequently deprotected to furnish the target MOGLs (FIG. 31d). Synthetic samples of the 2-O-acylated iglu #121 (25), iglu #101 (26), and iglu #401 (28) matched HPLC retention times and MS/MS spectra of the corresponding natural compounds (FIG. 31b, 8), confirming their structures. In all cases, these Cel-cest-1.2-dependent, 2-O-acylated glucosides have earlier HPLC retention time than their putative 6-O-acylated isomers, consistent with the previously reported retention time patterns of acylated uric acid glucosides.²³ Since Cel-cest-1.2 mutants are defective specifically in the production of the earlier eluting isomer of mono-acylated MOGLs, these observations indicate that Cel-CEST-1.2 is specifically required for 2-O-acylation of MOGLs (Table S4).

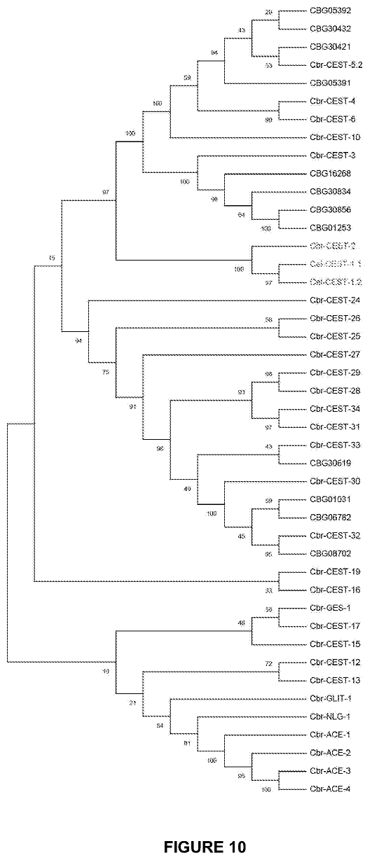

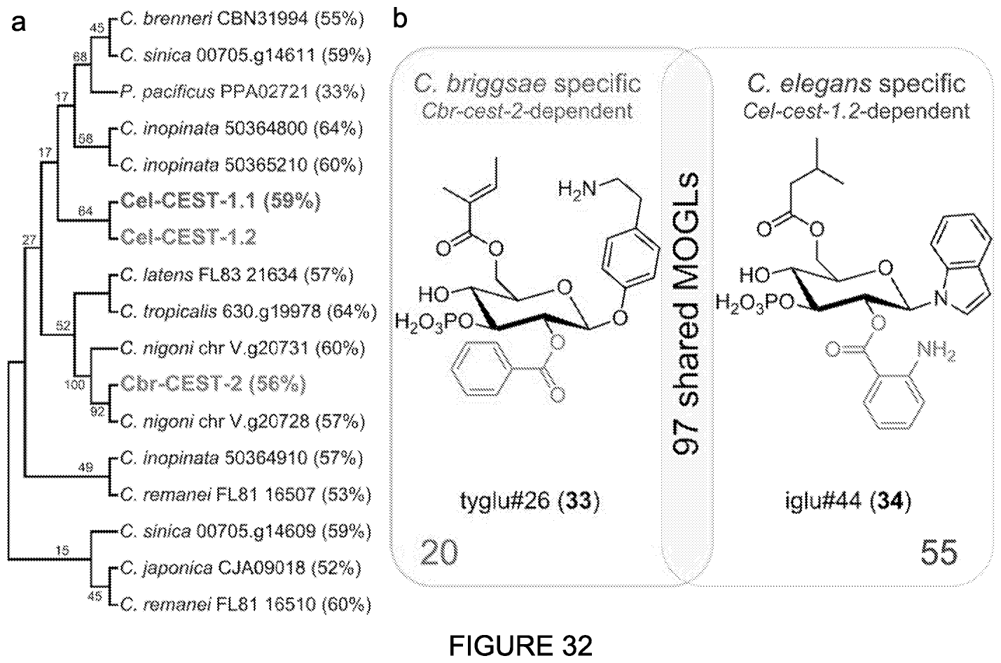

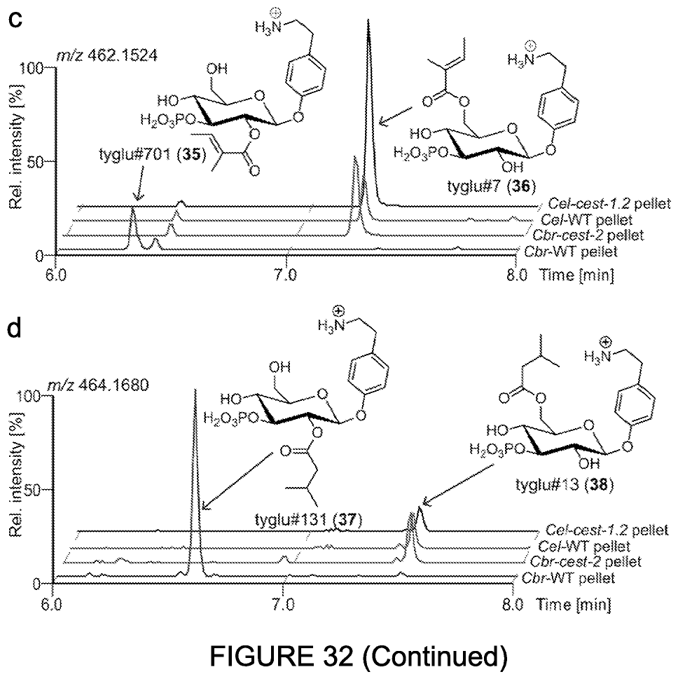

Cbr-CEST-2 is the functional ortholog of Cel-CEST-1.2. Cel-CEST-1.2 appears to be well conserved across the genus Caenorhabditis and possibly other nematode genera, e.g. Pristionchus (FIG. 32a). We recently showed that MOGLs are also produced by C. briggsae, a species closely related to C. elegans, and that MOGL biosynthesis in C. briggsae also requires the LROs.⁷ Similar to C. elegans, the C. briggsae genome encodes a large family of carboxylesterase homologs, including Cbr-CEST-2, which has the highest sequence similarity to Cel-CEST-1.2 (FIG. 10).²⁴ Therefore, we hypothesized that the production of a subset of MOGLs, including any Cel-cest-1.2-dependent compounds also produced by C. briggsae, may require Cbr-CEST-2. Like Cel-CEST-1.2, Cbr-CEST-2 includes a C-terminal transmembrane domain and the conserved active site serine (FIGS. 1, 2).

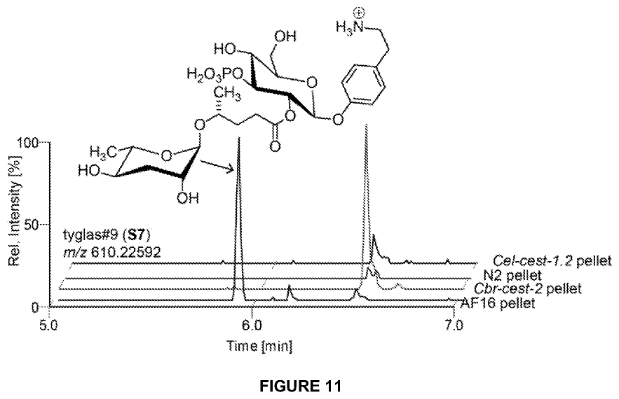

Using CRISPR/Cas9, we generated two Cbr-cest-2 null mutant strains and compared their endo- and exo-metabolomes with C. briggsae wildtype via HPLC-HRMS-based comparative metabolomics, as above. We found that Cbr-cest-2 mutants are defective in the production of >150 different MOGLs, including 97 MOGLs also produced by C. elegans, all of which are Cel-cest-1.2-dependent (FIG. 32a, b, Table S4). These data suggest that, like Cel-CEST-1.2, Cbr-CEST-2 is specifically required for 2-O-acylation in MOGL biosynthesis (FIG. 32b). We further detected several Cbr-cest-2-dependent MOGLs that are specific to C. briggsae. For example, Cbr-cest-2 mutants are defective in the biosynthesis of ascaroside-containing tyramine glucosides (e.g. tyglas #9, S7), which are not produced in C. elegans (FIG. 11). Similarly, C. briggsae produce two isomers of tigloyl or isovaleroyl-modified tyglu glucosides, of which only the earlier eluting peak is Cbr-cest-2-dependent (tyglu #701 35, tyglu #131 37) (FIG. 32c, d), whereas C. elegans only produce the later eluting isomer, which is Cel-cest-1.2-independent and thus likely represent the 6-O-acylated variant (FIG. 32c, d). Taken together, these findings indicate that Cel-CEST-1.2 and Cbr-CEST-2 represent functional orthologs with highly similar substrate ranges and are required for 2-O-acylation of a range of scaffold glucosides.

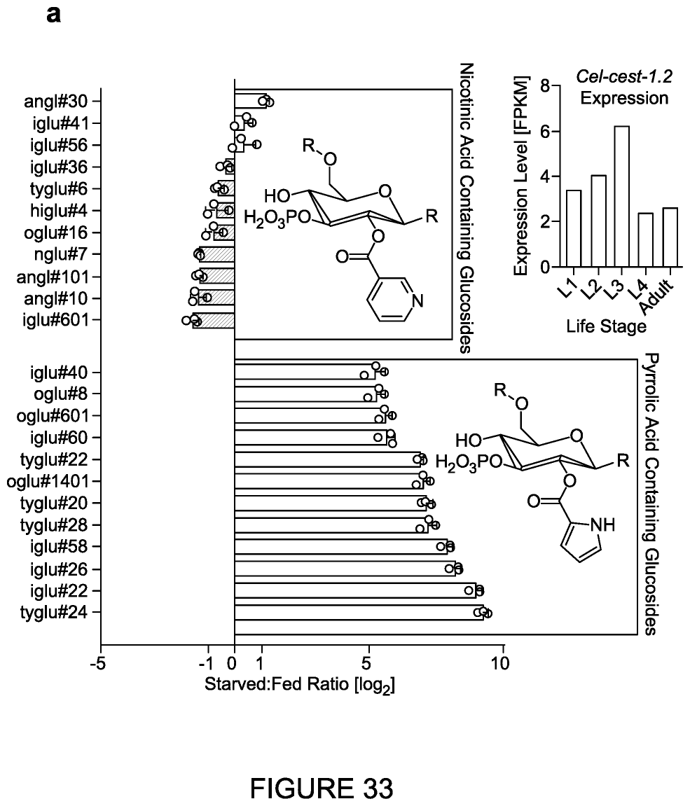

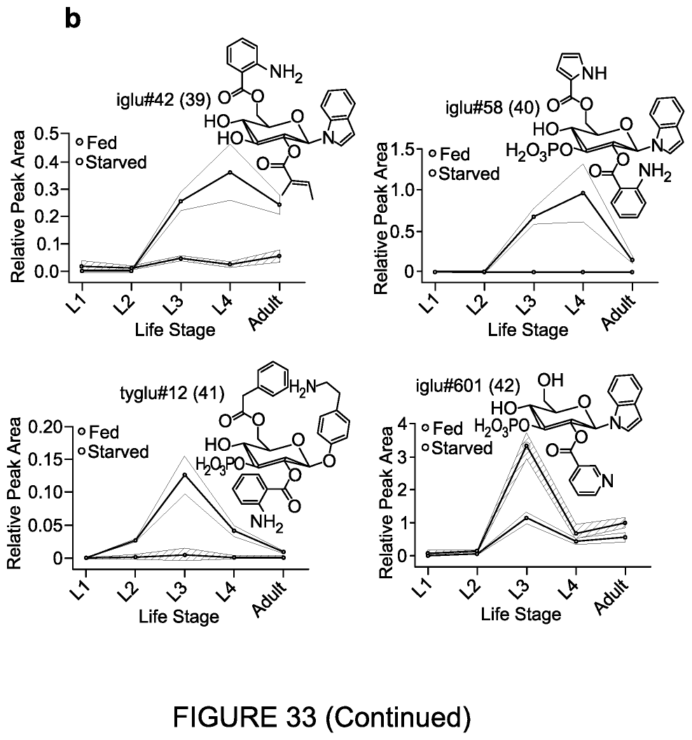

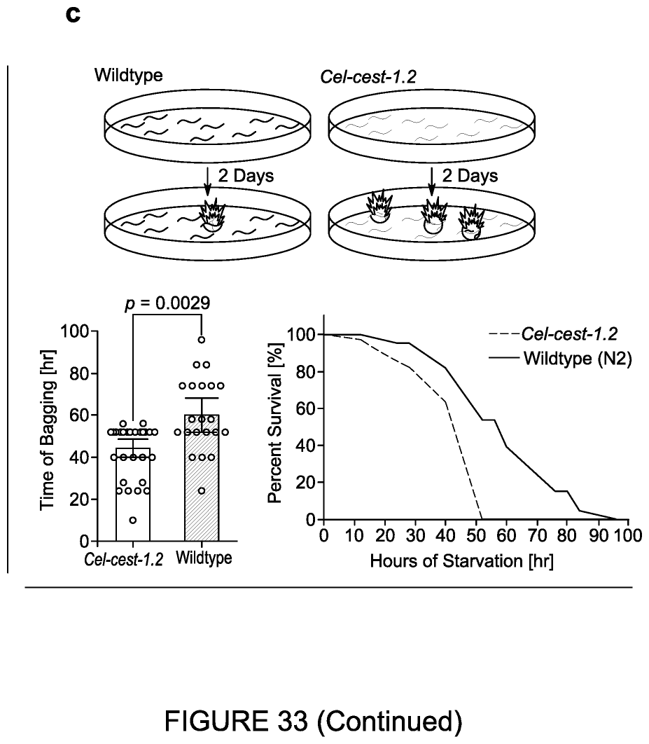

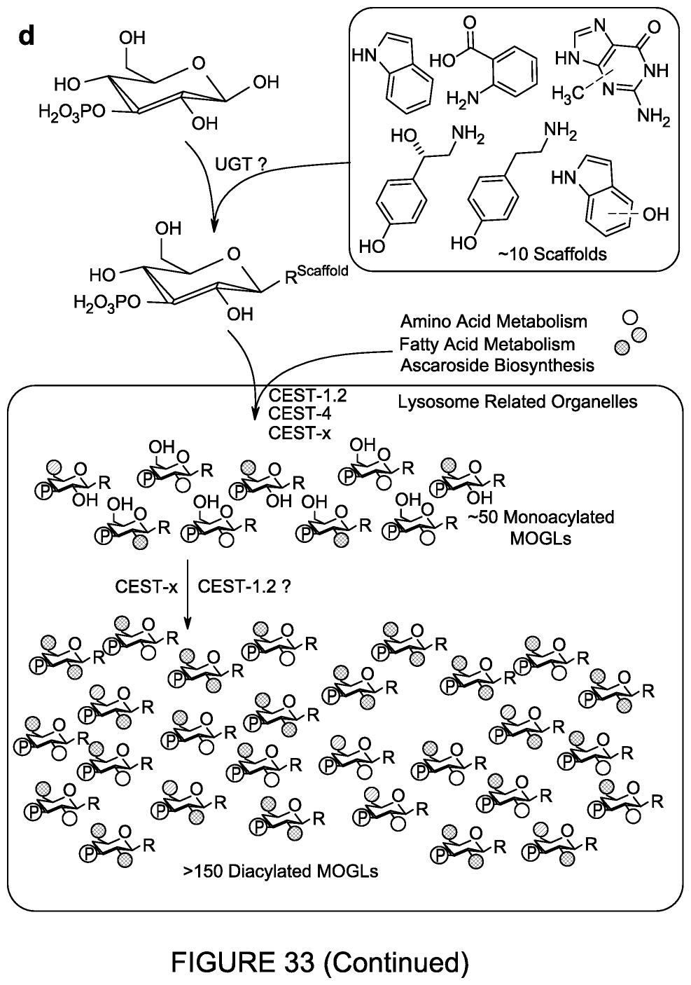

Lifestage- and starvation-dependent roles of Cel-CEST-1.2. Biosynthesis of small molecules in C. elegans is often strongly dependent on developmental stage and nutritional state.²⁵⁻²⁷ Previous transcriptomic analysis showed that Cel-cest-1.2 expression peaks at the third larval stage (L3) and is induced by starvation (FIGS. 30c, 33a).¹⁷ To investigate the effect of developmental stage and starvation on the production of Cel-cest-1.2-dependent MOGLs, we obtained endo-metabolome samples from all four larval stages as well as gravid adults, under nutrient-replete conditions and after 24 hr of starvation, followed by targeted analysis via HPLC-HRMS.

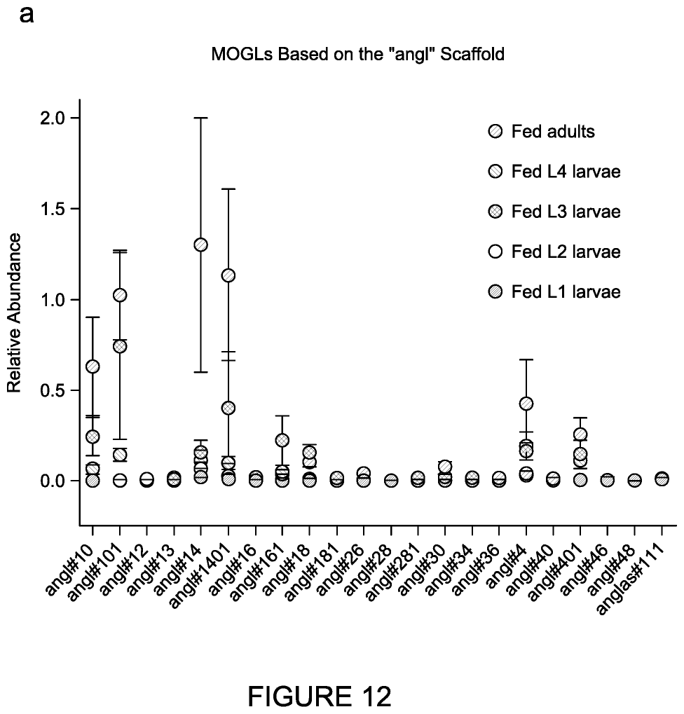

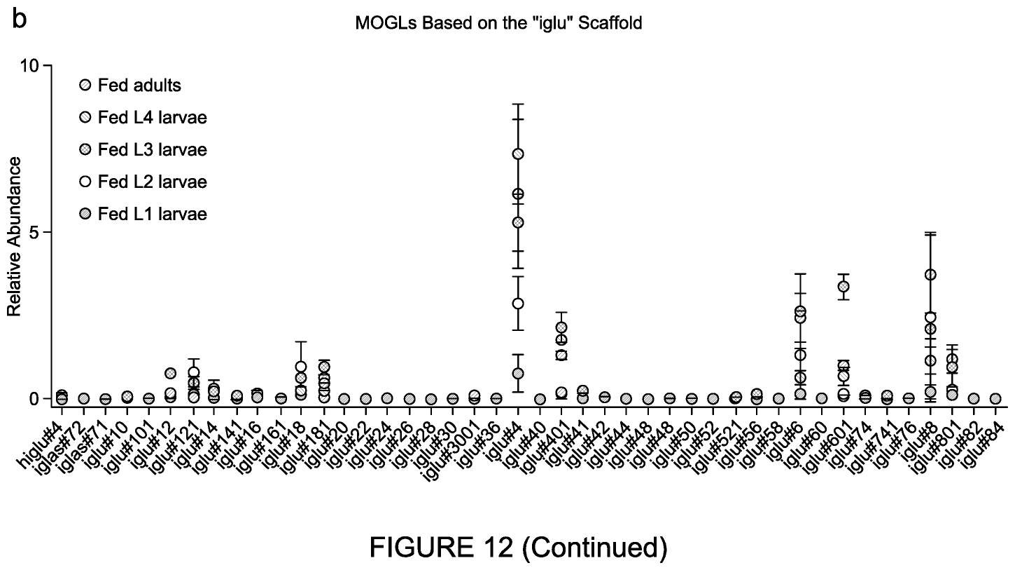

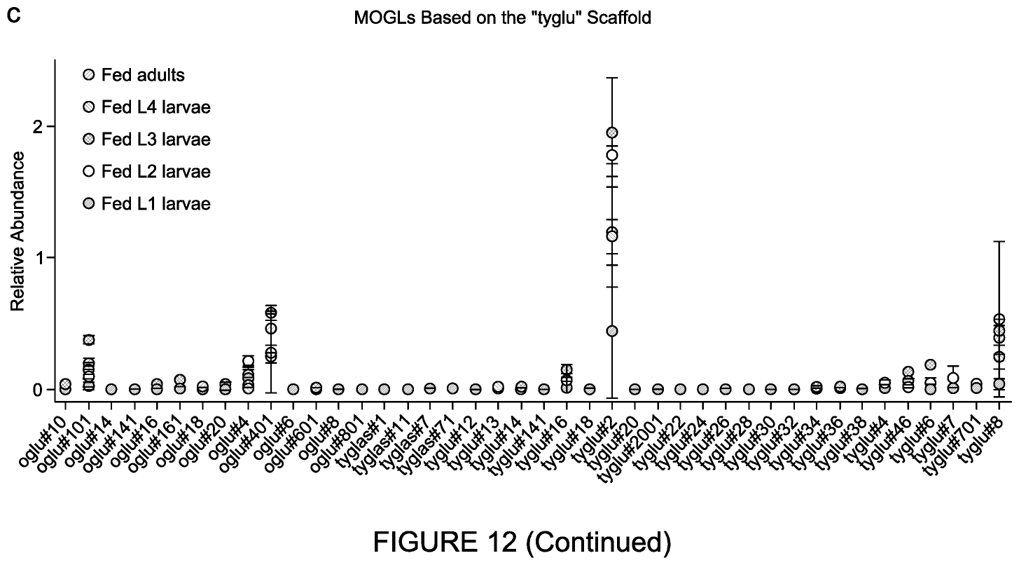

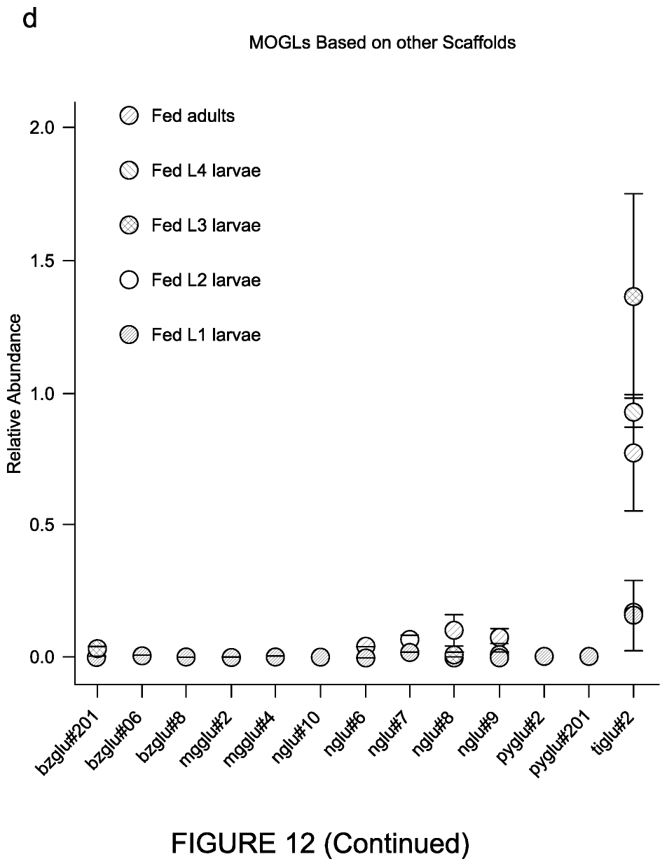

Biosynthesis of most Cel-cest-1.2-dependent MOGLs was strongly induced by starvation. Pyrrolic acid-containing MOGLs were most strongly upregulated (e.g. iglu #58 (40)), whereas MOGLs incorporating nicotinic acid were not increased or even slightly downregulated (e.g. iglu #601 (42)) FIG. 33a-b, 12-14). These trends were observed consistently across different glucose scaffolds (FIG. 33a, 12-14). In contrast, abundances of the unmodified scaffolds (e.g. iglu #2 (5)) were reduced during starvation, possibly due to lack of dietary input or because scaffold pools get depleted as a result of increased production of acylated MOGLs via Cel-CEST-1.2 and related CEST enzymes under these conditions (FIG. 12-14,). In addition, production of Cel-cest-1.2-dependent MOGLs was found to be strongly life stage-specific. Reflecting the expression pattern of Cel-cest-1.2 during development, Cel-cest-1.2-dependent metabolites were generally most abundant at the L3 larval stage; however, several compounds (e.g. iglu #42 (39) and iglu #58 (40)) showed alternate patterns with maximal production e.g. at the L4 larval stage (FIG. 33b, 12-14). Production of most Cel-cest-1.2-dependent MOGLs was increased by starvation in most tested developmental stages, except the L1 larval stage, where starvation seemed to have little effect.

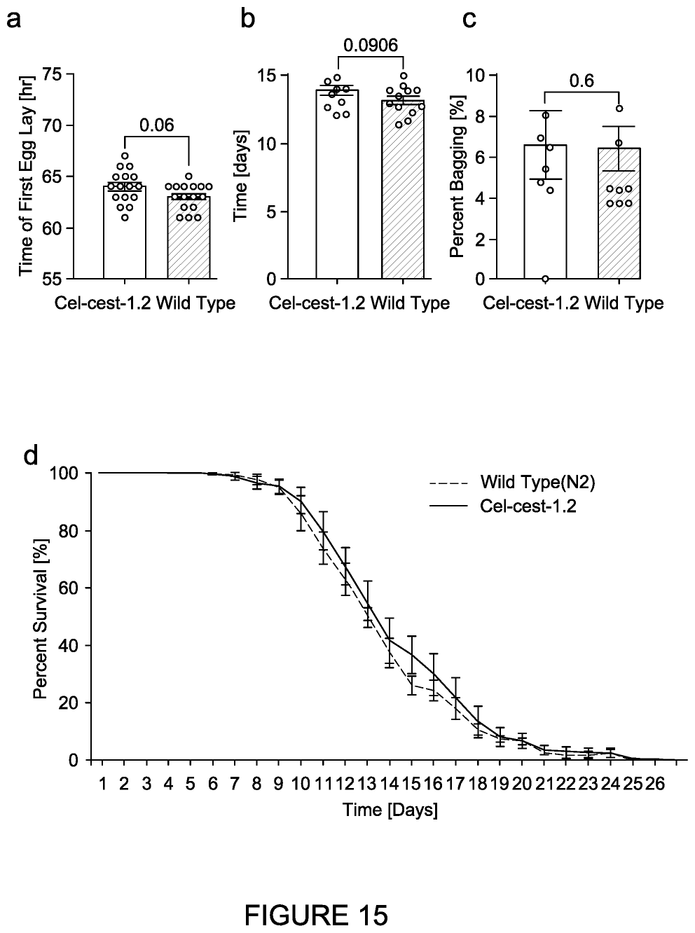

C. elegans is an important model for how starvation and dietary restriction affect lifespan in animals,²⁸⁻³¹ and small molecules have been shown to play a major role in the underlying mechanisms.³² Because MOGL biosynthesis is strongly upregulated during starvation, we tested whether Cel-CEST-1.2 is required for starvation survival (FIG. 33c). We found that lifespan of starved Cel-cest-1.2 adults was significantly reduced compared to wildtype (FIG. 4c), whereas there were no significant differences in development or lifespan under food-replete conditions (FIG. 15). Reduced lifespan during starvation of Cel-cest-1.2 animals was exclusively due to internal hatching of larvae, a matricide phenotype that results in bursting of the worm body, known as “bagging”³²⁻³⁵

These results demonstrate that Cel-CEST-1.2 and Cbr-CEST-2 are required for 2-O-acylation in the biosynthetic pathways of >150 different MOGLs. The product ranges in the two nematode species largely overlap, and differences may be due primarily to differences in available substrate pools. Despite the very large number of Cel-CEST-1.2/Cbr-CEST-2-dependent metabolites, their biosynthetic roles appear to be specific to 2-O-acylation, since every significant metabolic feature strongly downregulated or abolished in Cel-cest-1.2 or Cbr-cest-2 mutants, as detected in our comparative metabolomic analysis, could be assigned to a 2-O-acylated glucoside. Members of the α/β hydrolase family are known to exhibit broad substrate promiscuity,³⁶ for example, the human Cel-CEST-1.2 homolog, carboxylesterase 2 (CES2) is capable of cleaving a diverse range of xenobiotics.³⁷

In conjunction with the previous finding that Cel-cest-4 is specifically required for 6-O-attachment of anthranilate in indole glucosides (e.g. iglu #4 (11) in FIG. 30b), our results for Cel-cest-1.2 or Cbr-cest-2 mutants allow proposing a combinatorial model for MOGL biosynthesis (FIG. 33d). Following assembly of the glucoside scaffolds from indole, neurotransmitters (e.g. tyramine, octopamine) and other building blocks via UDP-glucuronosyltransferases, a wide range of acyl moieties are attached to the 2-position of glucose via Cel-cest-1.2 or the 6-position via Cel-cest-4 and additional homologs. Attachment of a second acyl moiety to produce diacylated MOGLs likely involves additional CEST-homologs.

Whereas none of the abundant diacylated MOGLs are strictly cest-4-dependent,⁷ production of a large number of diacylated MOGLs is fully abolished in Cel-cest-1.2 mutants, suggesting that Cel-CEST-1.2 is primarily responsible for 2-O-acylation, whereas there must be additional homologs mediating 6-O-acylation, in addition to Cel-CEST-4, which compared to Cel-CEST-1.2, appears to have a much narrower substrate scope. Attempts to recapitulate the biosynthetic activities of CESTs in vitro have been unsuccessful so far, likely due to the presence of the C-terminal transmembrane domain which may cause improper folding under in vitro conditions.^{7,9,38}

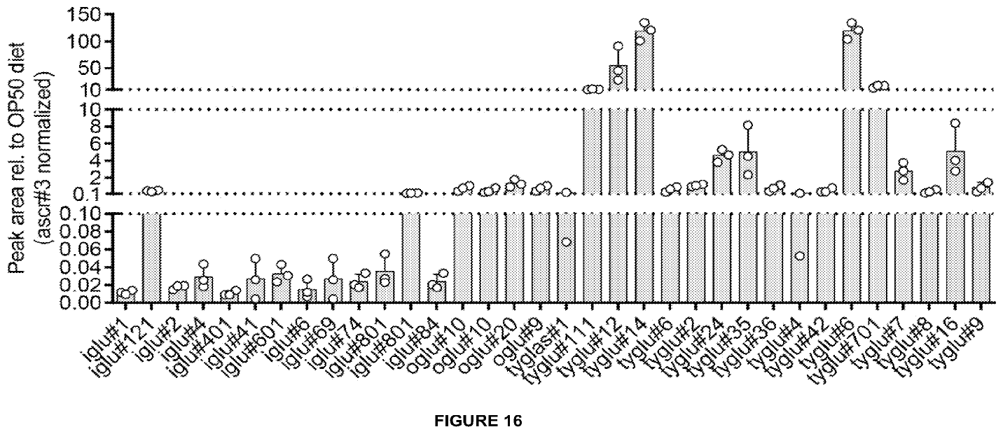

Our results further demonstrate that MOGL biosynthesis is highly regulated during development and depends on nutritional conditions. Different compound profiles at different life stages likely result in part from regulation of cest-expression, but may also reflect changes in substrate pools. For example, starvation is generally associated with increased protein turnover, which may result in an increase in amino acid degradation-derived building blocks, e.g. pyrrolic acid from proline or isovaleric and tiglic acid from leucine and isoleucine, respectively.^{39,40} Further, the relatives abundance of MOGLs may also depend on bacterial metabolism.²² For example, most bacteria occurring naturally with C. elegans produce much smaller amounts of indole than E. coli OP50.⁴¹ Correspondingly, we observed that C. elegans fed Providencia alcalifaciens JUb39, a bacterial species found with C. elegans in the wild, produce less indole-derived MOGLs compared to OP50-fed worms, whereas production of tyramine-derived MOGLs is increased, consistent with increased tyramine production in C. elegans fed Jub39 bacteria (FIG. 16).^{22,42}

Notably, MOGLs are mostly retained in the worm body and not excreted, suggesting that they serve specific intra-organismal function(s), paralleling the role of ascarosides in inter-organismal signaling. Their highly context-specific production further supports the hypothesis that MOGLs may serve diverse biological functions. Our finding that Cel-cest-1.2 plays an important role for starvation survival and is conserved across other species provides a starting point for elucidating the role of MOGLs in C. elegans and other nematodes.

### REFERENCES

(1) Schroeder, F. C. Modular Assembly of Primary Metabolic Building Blocks: A Chemical Language in C. Elegans. Chem. Biol. 2015, 22 (1), 7-16. doi.org/10.1016/j.chembiol.2014.10.012.
(2) Butcher, R. A. Decoding Chemical Communication in Nematodes. Nat. Prod. Rep. 2017, 34 (5), 472-477. doi.org/10.1039/C₇NP00007C.
(3) Butcher, R. A.; Fujita, M.; Schroeder, F. C.; Clardy, J. Small-Molecule Pheromones That Control Dauer Development in Caenorhabditis Elegans. Nat. Chem. Biol. 2007, 3 (7), 420-422. doi.org/10.1038/nchembio.2007.3.
(4) Jeong, P. Y.; Jung, M.; Yim, Y. H.; Kim, H.; Park, M.; Hong, E.; Lee, W.; Kim, Y. H.; Kim, K.; Paik, Y. K. Chemical Structure and Biological Activity of the Caenorhabditis Elegans Dauer-Inducing Pheromone. Nature 2005, 433 (7025), 541-545. doi.org/10.1038/nature03201.
(5) Yu, Y.; Zhang, Y. K.; Manohar, M.; Artyukhin, A. B.; Kumari, A.; Tenjo-Castano, F. J.; Nguyen, H.; Routray, P.; Choe, A.; Klessig, D. F.; Schroeder, F. C. Nematode Signaling Molecules Are Extensively Metabolized by Animals, Plants, and Microorganisms. ACS Chem. Biol. 2021, 16 (6), acschembio.1c00217. doi.org/10.1021/acschembio.lc00217.
(6) Manosalva, P.; Manohar, M.; von Reuss, S. H.; Chen, S.; Koch, A.; Kaplan, F.; Choe, A.; Micikas, R. J.; Wang, X.; Kogel, K.-H.; Sternberg, P. W.; Williamson, V. M.; Schroeder, F. C.; Klessig, D. F. Conserved Nematode Signalling Molecules Elicit Plant Defenses and Pathogen Resistance. Nat. Commun. 2015, 6 (1), 7795. doi.org/10.1038/ncomms8795.
(7) Le, H. H.; Wrobel, C. J. J.; Cohen, S. M.; Yu, J.; Park, H.; Helf, M. J.; Curtis, B. J.; Kruempel, J. C.; Rodrigues, P. R.; Hu, P. J.; Sternberg, P. W.; Schroeder, F. C. Modular Metabolite Assembly in Caenorhabditis Elegans Depends on Carboxylesterases and Formation of Lysosome-Related Organelles. Elife 2020, 9, 1-42. doi.org/10.7554/eLife.61886.
(8) Falcke, J. M.; Bose, N.; Artyukhin, A. B.; Rödelsperger, C.; Markov, G. V.; Yim, J. J.; Grimm, D.; Claassen, M. H.; Panda, O.; Baccile, J. A.; Zhang, Y. K.; Le, H. H.; Jolic, D.; Schroeder, F. C.; Sommer, R. J. Linking Genomic and Metabolomic Natural Variation Uncovers Nematode Pheromone Biosynthesis. Cell Chem. Biol. 2018, 25 (6), 787-796.e12. doi.org/10.1016/J.CHEMBIOL.2018.04.004.
(9) Faghih, N.; Bhar, S.; Zhou, Y.; Dar, A. R.; Mai, K.; Bailey, L.; Basso, K. B.; Butcher, R. A. A Large Family of Enzymes Responsible for the Modular Architecture of Nematode Pheromones. J. Am. Chem. Soc. 2020, 142 (32), jacs.0c04223. doi.org/10.1021/jacs.0c04223.
(10) Chen, A. L.; Lum, K. M.; Lara-Gonzalez, P.; Ogasawara, D.; Cognetta, A. B.; To, A.; Parsons, W. H.; Simon, G. M.; Desai, A.; Petrascheck, M.; Bar-Peled, L.; Cravatt, B. F. Pharmacological Convergence Reveals a Lipid Pathway That Regulates C. Elegans Lifespan. Nat. Chem. Biol. 2019, 15 (5), 453-462. doi.org/10.1038/s41589-019-0243-4.
(11) Long, J. Z.; Cravatt, B. F. The Metabolic Serine Hydrolases and Their Functions in Mammalian Physiology and Disease. Chemical Reviews. NIH Public Access Oct. 12, 2011, pp 6022-6063. doi.org/10.1021/cr200075y.
(12) Hermann, G. J.; Schroeder, L. K.; Hieb, C. A.; Kershner, A. M.; Rabbitts, B. M.; Fonarev, P.; Grant, B. D.; Priess, J. R. Genetic Analysis of Lysosomal Trafficking in Caenorhabditis Elegans. Mol. Biol. Cell 2005, 16 (7), 3273-3288. doi.org/10.1091/mbc.E05-01-0060.
(13) O'Donnell, M. P.; Fox, B. W.; Chao, P. H.; Schroeder, F. C.; Sengupta, P. A Neurotransmitter Produced by Gut Bacteria Modulates Host Sensory Behaviour. Nature 2020, 583 (7816), 415-420. doi.org/10.1038/s41586-020-2395-5.
(14) Stupp, G. S.; von Reuss, S. H.; Izrayelit, Y.; Ajredini, R.; Schroeder, F. C.; Edison, A. S. Chemical Detoxification of Small Molecules by Caenorhabditis Elegans. ACS Chem. Biol. 2013, 8 (2), 309-313. doi.org/10.1021/cb300520u.
(15) Hastings, J.; Mains, A.; Virk, B.; Rodriguez, N.; Murdoch, S.; Pearce, J.; Bergmann, S.; Le Novbre, N.; Casanueva, O. Multi-Omics and Genome-Scale Modeling Reveal a Metabolic Shift During C. Elegans Aging. Front. Mol. Biosci. 2019, 6, 2. doi.org/10.3389/fmolb.2019.00002.
(16) Coburn, C.; Allman, E.; Mahanti, P.; Benedetto, A.; Cabreiro, F.; Pincus, Z.; Matthijssens, F.; Araiz, C.; Mandel, A.; Vlachos, M.; Edwards, S. A.; Fischer, G.; Davidson, A.; Pryor, R. E.; Stevens, A.; Slack, F. J.; Tavernarakis, N.; Braeckman, B. P.; Schroeder, F. C.; Nehrke, K.; Gems, D. Anthranilate Fluorescence Marks a Calcium-Propagated Necrotic Wave That Promotes Organismal Death in C. Elegans. PLoS Biol. 2013, 11 (7). doi.org/10.1371/joumal.pbio.1001613.
(17) Harris, T. W.; Arnaboldi, V.; Cain, S.; Chan, J.; Chen, W. J.; Cho, J.; Davis, P.; Gao, S.; Grove, C. A.; Kishore, R.; Lee, R. Y. N.; Muller, H.-M.; Nakamura, C.; Nuin, P.; Paulini, M.; Raciti, D.; Rodgers, F. H.; Russell, M.; Schindelman, G.; Auken, K. V; Wang, Q.; Williams, G.; Wright, A. J.; Yook, K.; Howe, K. L.; Schedl, T.; Stein, L.; Sternberg, P. W. WormBase: A Modem Model Organism Information Resource. Nucleic Acids Res. 2020, 48 (D1), D762-D767. doi.org/10.1093/NAR/GKZ920.
(18) Tautenhahn, R.; Patti, G. J.; Rinehart, D.; Siuzdak, G. XCMS Online: A Web-Based Platform to Process Untargeted Metabolomic Data. Anal. Chem. 2012, 84 (11), 5035-5039. doi.org/10.1021/ac300698c.
(19) Artyukhin, A. B.; Zhang, Y. K.; Akagi, A. E.; Panda, O.; Sternberg, P. W.; Schroeder, F. C. Metabolomic “Dark Matter” Dependent on Peroxisomal β-Oxidation in Caenorhabditis Elegans. J. Am. Chem. Soc. 2018, 140 (8), 2841-2852. doi.org/10.1021/jacs.7b11811.
(20) von Reuss, S. H.; Bose, N.; Srinivasan, J.; Yim, J. J.; Judkins, J. C.; Sternberg, P. W.; Schroeder, F. C. Comparative Metabolomics Reveals Biogenesis of Ascarosides, a Modular Library of Small-Molecule Signals in C. Elegans. J. Am. Chem. Soc. 2012, 134 (3), 1817-1824. doi.org/10.1021/ja210202y.
(21) Messaoudi, S.; Sancelme, M.; Polard-Housset, V.; Aboab, B.; Moreau, P.; Prudhomme, M. Synthesis and Biological Evaluation of Oxindoles and Benzimidazolinones Derivatives. Eur. J. Med. Chem. 2004, 39 (5), 453-458. doi.org/10.1016/j.ejmech.2004.01.001.
(22) O'Donnell, M. P.; Fox, B. W.; Chao, P.-H.; Schroeder, F. C.; Sengupta, P. A Neurotransmitter Produced by Gut Bacteria Modulates Host Sensory Behaviour. Nature 2020, 1-6. doi.org/10.1038/s41586-020-2395-5.
(23) Curtis, B. J.; Kim, L. J.; Wrobel, C. J. J.; Eagan, J. M.; Smith, R. A.; Burch, J. E.; Le, H. H.;
Artyukhin, A. B.; Nelson, H. M.; Schroeder, F. C. Identification of Uric Acid Gluconucleoside-Ascaroside Conjugates in Caenorhabditis Elegans by Combining Synthesis and MicroED. Org. Lett. 2020, 22 (17). doi.org/10.1021/acs.orglett.0c02038.
(24) Kanzaki, N.; Tsai, I. J.; Tanaka, R.; Hunt, V. L.; Liu, D.; Tsuyama, K.; Maeda, Y.; Namai, S.; Kumagai, R.; Tracey, A.; Holroyd, N.; Doyle, S. R.; Woodruff, G. C.; Murase, K.; Kitazume, H.; Chai, C.; Akagi, A.; Panda, O.; Ke, H. M.; Schroeder, F. C.; Wang, J.; Berriman, M.; Sternberg, P. W.; Sugimoto, A.; Kikuchi, T. Biology and Genome of a Newly Discovered Sibling Species of Caenorhabditis Elegans. Nat. Commun. 2018, 9 (1), 1-12. doi.org/10.1038/s41467-018-05712-5.
(25) Artyukhin, A. B.; Yim, J. J.; Srinivasan, J.; Izrayelit, Y.; Bose, N.; von Reuss, S. H.; Jo, Y.; Jordan, J. M.; Baugh, L. R.; Cheong, M.; Sternberg, P. W.; Avery, L.; Schroeder, F. C. Succinylated Octopamine Ascarosides and a New Pathway of Biogenic Amine Metabolism in Caenorhabditis Elegans. J. Biol. Chem. 2013, 288 (26), 18778-18783. doi.org/10.1074/jbc.C_{113.477000.}
(26) Ludewig, A. H.; Artyukhin, A. B.; Aprison, E. Z.; Rodrigues, P. R.; Pulido, D. C.; Burkhardt, R. N.; Panda, O.; Zhang, Y. K.; Gudibanda, P.; Ruvinsky, I.; Schroeder, F. C. An Excreted Small Molecule Promotes C. Elegans Reproductive Development and Aging. Nat. Chem. Biol. 2019, 15 (8), 838-845. doi.org/10.1038/s41589-019-0321-7.
(27) Hoki, J. S.; Le, H. H.; Mellott, K. E.; Zhang, Y. K.; Fox, B. W.; Rodrigues, P. R.; Yu, Y.; Helf, M. J.; Baccile, J. A.; Schroeder, F. C. Deep Interrogation of Metabolism Using a Pathway-Targeted Click-Chemistry Approach. J. Am. Chem. Soc. 2020, 142 (43), 18449-18459. doi.org/10.1021/jacs.0c06877.
(28) Lee, G. D.; Wilson, M. A.; Zhu, M.; Wolkow, C. A.; De Cabo, R.; Ingram, D. K.; Zou, S. Dietary Deprivation Extends Lifespan in Caenorhabditis Elegans. Aging Cell 2006, 5 (6), 515-524. doi.org/10.1111/j.1474-9726.2006.00241.x.
(29) Lakowski, B.; Hekimi, S. The Genetics of Caloric Restriction in Caenorhabditis Elegans. Proc. Natl. Acad. Sci. U.S.A 1998, 95 (22), 13091-13096. doi.org/10.1073/pnas.95.22.13091.
(30) Houthoofd, K.; Braeckman, B. P.; Lenaerts, I.; Brys, K.; De Vreese, A.; Van Eygen, S.; Vanfleteren, J. R. Axenic Growth Up-Regulates Mass-Specific Metabolic Rate, Stress Resistance, and Extends Life Span in Caenorhabditis Elegans. Exp. Gerontol. 2002, 37 (12), 1371-1378. doi.org/10.1016/S0531-5565(02)00173-0.
(31) Houthoofd, K.; Braeckman, B. P.; Johnson, T. E.; Vanfleteren, J. R. Life Extension via Dietary Restriction Is Independent of the Ins/IGF-1 Signalling Pathway in Caenorhabditis Elegans Exp. Gerontol. 2003, 38 (9), 947-954. doi.org/10.1016/S0531-5565(03)00161-X.
(32) Baugh, L. R.; Hu, P. J. Starvation Responses throughout the Caenorhabditis Elegans Life Cycle. Genetics. Genetics Society of America Dec. 1, 2020, pp 837-878. doi.org/10.1534/genetics.120.303565.
(33) Chen, J.; Caswell-Chen, E. P. Facultative Vivipary Is a Life-History Trait in Caenorhabditis Elegans. J. Nematol. 2004, 36 (2), 107-113.
(34) Seidel, H. S.; Kimble, J. The Oogenic Germline Starvation Response in C. Elegans. PLoS One 2011, 6 (12), 28074. doi.org/10.1371/journal.pone.0028074.
(35) Angelo, G.; Van Gilst, M. R. Starvation Protects Germline Stem Cells and Extends Reproductive Longevity in C. Elegans. Science (80-.). 2009, 326 (5955), 954-958. doi.org/10.1126/science.1178343.
(36) Martinez-Martinez, M.; Coscolin, C.; Santiago, G.; Chow, J.; Stogios, P. J.; Bargiela, R.; Gertler, C.; Navarro-Fernindez, J.; Bollinger, A.; Thies, S.; Méndez-Garcia, C.; Popovic, A.; Brown, G.; Chernikova, T. N.; Garcia-Moyano, A.; Bjerga, G. E. K.; Pérez-Garcia, P.; Hai, T.; Del Pozo, M. V.; Stokke, R.; Steen, I. H.; Cui, H.; Xu, X.; Nocek, B. P.; Alcaide, M.; Distaso, M.; Mesa, V.; Peliez, A. I.; Sinchez, J.; Buchholz, P. C. F.; Pleiss, J.; Fernindez-Guerra, A.; Glöckner, F. O.; Golyshina, O. V.; Yakimov, M. M.; Savchenko, A.; Jaeger, K. E.; Yakunin, A. F.; Streit, W. R.; Golyshin, P. N.; Guallar, V.; Ferrer, M. Determinants and Prediction of Esterase Substrate Promiscuity Patterns. ACS Chem. Biol. 2018, 13 (1), 225-234. doi.org/10.1021/acschembio.7b00996.
(37) Imai, T.; Taketani, M.; Shii, M.; Hosokawa, M.; Chiba, K. Substrate Specificity of Carboxylesterase Isozymes and Their Contribution to Hydrolase Activity in Human Liver and Small Intestine. Drug Metab. Dispos. 2006, 34 (10), 1734-1741. doi.org/10.1 124/dmd.106.009381.
(38) Falcke, J. M.; Bose, N.; Artyukhin, A. B.; Rödelsperger, C.; Markov, G. V.; Yim, J. J.; Grimm, D.; Claassen, M. H.; Panda, O.; Baccile, J. A.; Zhang, Y. K.; Le, H. H.; Jolic, D.; Schroeder, F. C.; Sommer, R. J. Linking Genomic and Metabolomic Natural Variation Uncovers Nematode Pheromone Biosynthesis. Cell Chem. Biol. 2018, 25 (6), 787-796.e12. doi.org/10.1016/j.chembiol.2018.04.004.
(39) Jones, C. B.; Ott, E. M.; Keener, J. M.; Curtiss, M.; Sandrin, V.; Babst, M. Regulation of Membrane Protein Degradation by Starvation-Response Pathways. Traffic 2012, 13 (3), 468-482. doi.org/10.1111/j.1600-0854.2011.01314.x.
(40) Gretzmeier, C.; Eiselein, S.; Johnson, G. R.; Engelke, R.; Nowag, H.; Zarei, M.; Kuttner, V.; Becker, A. C.; Rigbolt, K. T. G.; Hoyer-Hansen, M.; Andersen, J. S.; Mnnz, C.; Murphy, R. F.; Dengjel, J. Degradation of Protein Translation Machinery by Amino Acid Starvation-Induced Macroautophagy. Autophagy 2017, 13 (6), 1064-1075. doi.org/10.1080/15548627.2016.1274485.
(41) Lee, J. H.; Lee, J. Indole as an Intercellular Signal in Microbial Communities. FEMS Microbiology Reviews. 2010. doi.org/10.1111/j.1574-6976.2009.00204.x.
(42) Samuel, B. S.; Rowedder, H.; Braendle, C.; Fdlix, M.-A.; Ruvkun, G. Caenorhabditis Elegans Responses to Bacteria from Its Natural Habitats. Proc. Natl. Acad. Sci. 2016, 113 (27), E3941-E3949. doi.org/10.1073/PNAS.1607183113.

### A. Materials and Methods

Nematode and bacterial strains. Unless indicated otherwise, worms were maintained on Nematode Growth Medium (NGM) 6 cm diameter Petri dish plates with E. coli OP50 (www.wormbook.org/methods).¹ Nematode strains used in this study are listed below:

| Genotype | Strain | Allele | Source |
|---|---|---|---|
| Cel-cest-1.2 | PHX3928 | syb3928 | SunyBiotech |
| CBG04745 (Cbr-cest-2) | PS9060 | sy1616 | This work |
| CBG04745 (Cbr-cest-2) | PS8061 | sy1617 | This work |
| C. elegans wildtype | N2 | | Caenorhabditis Genetics Center (CGC) |
| C. briggsae wildtype | AF16 | | Caenorhabditis Genetics Center (CGC) |
| Cel-glo-1 | DH10 | zu437 | Caenorhabditis Genetics Center (CGC) |
| Cbr-glo-1 | PS8515 | sy1382 | Le, 2020² |
| Cbr-glo-1 | PS8516 | sy1383 | Le, 2020² |
| Cel-daf-22 | RB859 | ok693 | Caenorhabditis Genetics Center (CGC) |
| Cbr-daf-22 | PS8777 | sy1524 | Cohen et. al., 2021 (in prep) |
| Cbr-daf-22 | PS8778 | sy1525 | Cohen et. al., 2021 (in prep) |

Metabolite nomenclature. All newly detected metabolites for which a structure could be proposed were named using SMIDs. SMIDs (Small Molecule IDentifiers) have been introduced as a search-compatible naming system for metabolites newly identified from C. elegans and other nematodes. The SMID database (www.smid-db.org) is an electronic resource maintained in collaboration with WormBase (www.wormbase.org). A complete list of SMIDs can be found at www.smid-db.org/browse.

Amino acid sequence alignment. Alignments of Cel-CEST-1.1 with Cel-CEST-1.2 and Cbr-CEST-2 were done using T-Coffee Multiple Sequence alignment.³ Protein sequences are from WormBase. Amino acids were colored based on chemical properties: AVFPMILW=red (small+hydrophobic), DE=blue (acidic), RHK=magenta (basic), STYHCNGQ=green (hydroxyl+sulfhydryl+amine+glycine).

C. briggsae phylogenetic tree. The protein sequence of Cel-CEST-1.1 was submitted to an NCBI BLASTp search (restricted to species C. briggsae, conditional compositional BLOSUM62, gap open cost: 11, gap extension cost: 1, word size: 6).⁴ The top 36 BLAST hits by E-value and only the best scoring transcript variant was kept for each protein sequence hit. These 42 hits along with the 8 C. elegans esterase strains were then imported into MEGAX and aligned using MUSCLE⁵ (settings: gap open penalty: −2.9, gap extend 0, hydrophobicity multiplier 1.2, max. iterations 8, clustering method for all iterations: UPGMB, minimal diagonal length: 24). The evolutionary history was inferred using the Neighbor-Joining method.⁶ The optimal tree is shown. The percentage of replicate trees in which the associated taxa clustered together in the bootstrap test (200 replicates) are shown next to the branches.⁷ The evolutionary distances were computed using the JTT matrix-based method⁸ and are in the units of the number of amino acid substitutions per site. This analysis involved 44 amino acid sequences. All ambiguous positions were removed for each sequence pair (pairwise deletion option). There were a total of 1248 positions in the final dataset. Evolutionary analyses were conducted in MEGA X.⁹

Caenorhabditis Cel-CEST-1.1 homologs tree. The protein sequence of Cel-CEST-1.1 was submitted to an NCBI BLASTp search (restricted to various Caenorhabditis species, conditional compositional BLOSUM62, gap open coast:11, gap extension cost: 1, word size: 6).⁴ Hits with Bit-score above ˜300 were kept for each species. These 17 sequences were then imported into MEGAX¹⁰ and aligned using MUSCLE⁵ (settings: gap open penalty: −2.9, gap extend 0, hydrophobicity multiplier 1.2, max. iterations 8, clustering method for all iterations: UPGMB, minimal diagonal length: 24). The evolutionary history was inferred using the Neighbor-Joining method.⁶ The bootstrap consensus tree inferred from 200 replicates is taken to represent the evolutionary history of the taxa analyzed.⁷ Branches corresponding to partitions reproduced in less than 50% bootstrap replicates are collapsed. The percentage of replicate trees in which the associated taxa clustered together in the bootstrap test (200 replicates) are shown next to the branches.⁷ The evolutionary distances were computed using the JTT matrix-based method and are in the units of the number of amino acid substitutions per site.⁸ This analysis involved 17 amino acid sequences. All ambiguous positions were removed for each sequence pair (pairwise deletion option). There were a total of 1803 positions in the final dataset. Evolutionary analyses were conducted in MEGA X.⁹

C. briggsae CRISPR mutagenesis for generation of Cbr-cest-2 null mutants. The Cbr-cest-2 mutants PS9060 and PS9061 were both created using the briggsae-adaptation of the STOP-IN cassette method as described previously.n^{11,12} Both strains were made via insertion of the STOP-IN cassette into the middle of the first exon using the guide CATTACTCATACAAGCTGGA.

Nematode cultures. Cultures were started by chunking C. elegans or C. briggsae onto 10 cm NGM plates (each seeded with 800 μL of OP50 E. coli grown to stationary phase in Lennox Broth) and incubated at 22° C. Once most food was consumed, each plate was washed with 25 mL of S-complete medium into a 125 mL Erlenmeyer flask, and 1 mL of OP50 E. coli was added (E. coli cultures were grown to stationary phase in Lennox Broth, pelleted and resuspended at 1 g wet mass per 1 mL M9 buffer), shaking at 220 RPM and 22° C. After 70 hr, cultures were centrifuged at 1000 g for 1 min. After discarding supernatant, 24 mL H₂O was added along with 6 mL bleach, 900 μL 10 M NaOH, and the mixture was shaken for 3 min to prepare eggs. Eggs were centrifuged at 1000 g, the supernatant was removed, and the egg pellet was washed with 25 mL M9 buffer twice and then suspended in a final volume of 5 mL M9 buffer in a 50 mL centrifuge tube. Eggs were counted and placed on a rocker and allowed to hatch as L1 larvae for 24 hr at 22° C. 70,000 L1 larvae were seeded in 25 mL cultures of S-complete with 1 mL of OP50 and incubated at 220 RPM and 22° C. in a 125 mL Erlenmeyer flask. After 72 hr, worms were spun down at 1000 g for 5 min, and media was separated from worm body pellet. Separated media and worm pellet were flash frozen over liquid nitrogen and then lyophilized. Two to four biological replicates were grown for each strain. Mutants were grown with parallel wildtype controls, and biological replicates were started on different days.

Nematode cultures with Providencia Jub39.¹³ Approximately 10,000 mixed stage C. elegans wildtype (N₂) animals were reared on either E. coli OP50 or Providencia alcalifaciens JUb39 at a density of 2,000 animals per 10 cm NGM plate. Animals were collected in 15 mL conical tubes by serially washing the plates with M9 buffer. Animals were washed three times with 10 mL M9 before transfer to 1.5 mL microfuge tubes, then snap frozen in liquid nitrogen. Samples were lyophilized for 18-24 hr using a VirTis BenchTop 4K Freeze Dryer. After the addition of two stainless steel grinding balls and 1 mL of 80% methanol, samples were sonicated for 5 min (2 sec on/off pulse cycle at 90 A) using a Qsonica Q700 Ultrasonic Processor with a water bath cup hom adaptor (Model 431C2). Following sonication, microfuge tubes were centrifuged at 10,000 g for 5 min in an Eppendorf 5417R centrifuge. 800 μL of the resulting supernatant was transferred to a clean 4 mL glass vial, and 800 μL of fresh methanol added to the sample. The sample was sonicated and centrifuged as described, and the resulting supernatant was transferred to the same receiver vial and concentrated to dryness in an SC250EXP Speedvac Concentrator coupled to an RVT5105 Refrigerated Vapor Trap (Thermo Scientific). The resulting powder was suspended in 120 μL of 100% methanol, followed by vigorous vortex and brief sonication. This solution was transferred to a clean microfuge tube and subjected to centrifugation at 20,000 g for 10 min in an Eppendorf 5417R centrifuge to remove precipitate. The resulting supernatant was transferred to an HPLC vial and analyzed by HPLC-MS.

Metabolite extraction. Lyophilized pellet or media samples were crushed and homogenized by shaking with 2.5 mm steel balls at 1300 RPM for 3 min in 30 s pulses while chilled with liquid nitrogen (SPEX sample prep miniG 1600). Powdered media and pellet samples were extracted with 10 mL methanol in 50 mL centrifuge tubes, rocking overnight at 22° C. Extractions were pelleted at 5000 g for 10 min at 4° C., and supernatants were transferred to 20 mL glass scintillation vials. Samples were then dried in a SpeedVac (Thermo Fisher Scientific) vacuum concentrator. Dried materials were resuspended in 1 mL methanol and vortexed for 1 min. Samples were pelleted at 10,000 g for 5 min at 22° C., and supernatants were transferred to 2 mL HPLC vials and dried in a SpeedVac vacuum concentrator. Samples were resuspended in 100 μL of methanol, transferred into 1.7 mL Eppendorf tubes, and centrifuged at 18,000 g for 20 min at 4° C. Clarified extracts were transferred to HPLC vials and stored at −20° C. until analysis.

Preparation of endo-metabolome samples from staged starved and fed cultures. 40,000 synchronized L1 larvae were added to 125 mL Erlenmeyer flasks containing 30 mL of S-complete medium. Worms were fed with 4 mL of concentrated OP50 and incubated at 20° C. with shaking at 160 RPM for: 12 hr (L1), 24 hr (L2), 32 hr (L3), 40 hr (L4) and 58 hr (gravid adults). For preparation of starved samples, each of the stages was starved for 24 hr after reaching their desired developmental stage in S-complete without OP50. After incubation for the desired time, liquid cultures were centrifuged (1000 g, 22° C., 1 min) and supernatants were collected. Supernatant was separated from intact OP50 by centrifuging (3000 g, 22° C., 5 min), and the resulting supernatants (exo-metabolome) were lyophilized. Lyophilized samples were homogenized with a dounce homogenizer in 10 mL methanol and extracted on a stirring plate (22° C., 12 hr). The resulting suspension was centrifuged (4000 g, 22° C., 5 min) to remove any precipitate before carefully transferred to HPLC vials. Three biological replicates were started on different days.

Mass spectrometric analysis. High resolution LC-MS analysis was performed on a Thermo Fisher Scientific Vanquish Horizon UHPLC System coupled with a Thermo Q Exactive hybrid quadrupole-orbitrap high-resolution mass spectrometer equipped with a HESI ion source. 1 μL of extract was injected and separated using a water-acetonitrile gradient on a Thermo Scientific Hypersil GOLD C18 column (150 mm×2.1 mm 1.9 um particle size 175 Å pore size, Thermo Scientific) and maintained at 40° C. Solvents were all purchased from Fisher Scientific as HPLC grade. Solvent A: 0.1% formic acid in water; solvent B: 0.1% formic acid in acetonitrile. A/B gradient started at 1% B for 3 min, then from 1% to 100% B over 20 min, 100% for 5 min, then down to 1% B for 3 min. Mass spectrometer parameters: 3.5 kV spray voltage, 380° C. capillary temperature, 300° C. probe heater temperature, 60 sheath flow rate, 20 auxiliary flow rate, 2.0 spare gas; S-lens RF level 50.0, resolution 240,000, m/z range 150-1000, AGC target 3e6. Instrument was calibrated with positive and negative ion calibration solutions (Thermo Fisher) Pierce LTQ Velos ESI pos/neg calibration solutions. Peak areas were determined using Xcalibur 2.3 QualBrowser version 2.3.26 (Thermo Scientific) using a 5 ppm window around the m/z of interest. HPLC-MS peak areas were normalized to the measured abundance of ascr #3 (www.smid-db.org/detail/ascr %233) in each sample for all graphs in this manuscript, except for FIG. 6a, where iglu #2 (5) was used to normalized peak areas, and FIG. 6, which reports the non-normalized measurements for select ascarosides as well as for the indole scaffolds iglu #1 (4) and iglu #2 (5).

Feature detection and characterization. LC-MS RAW files from each sample were converted to mzXML (centroid mode) using MSConvert (ProteoWizard), followed by analysis using the XCMS¹⁴ analysis feature in Metaboseek (metaboseek.com). Peak detection was carried out with the centWave algorithm¹⁵ values set as: 4 ppm, 320 peakwidth, 3 snthresh, 3100 prefilter, FALSE fitgauss, 1 integrate, TRUE firstBaselineCheck, 0 noise, wMean mzCenterFun, −0.005 mzdiff XCMS feature grouping values were set as: 0.2 minfrac, 2 bw, 0.002 mzwid, 500 max, 1 minsamp, FALSE usegroup. Metaboseek peak filling values set as: 5 ppm_m, 5 rtw, TRUE rtrange. Resulting tables were then processed with the Metaboseek Data Explorer. Molecular features were filtered for each particular null mutant against all other mutants. Filter values were set as: 10 to max minFoldOverCtrl, 15000 to max meanInt, 120 to 1500 rt, 0.95 to max Peak Quality as calculated by Metaboseek. Features were then manually curated by removing isotopic and adducted redundancies. Remaining masses were put on the inclusion list for MS/MS (ddMS2) characterization. Positive and negative mode data were processed separately. In both cases we checked if a feature had a corresponding peak in the opposite ionization mode, since fragmentation spectra in different modes often provide complementary structural information. To acquire MS/MS spectra, we ran a top-10 data dependent MS2 method on a Thermo QExactive-HF mass spectrometer with MS1 resolution 60,000, AGC target 1×10\hat{ }6, maximum IT (injection time) 50 ms, MS/MS resolution 45,000, AGC target 5×10\hat{ }5, maximum IT 80 ms, isolation window 1.0 m/z, stepped NCE (normalized collision energy) 25, 50, dynamic exclusion 3 s.

Starvation survival assay. 20-30 gravid adults were placed on 6 cm NGM plates seeded with 75 μL OP50 bacteria grown overnight in LB media (ad libitum, AL plates) and allowed to lay eggs for 2 hr. 15-20 single embryos were isolated onto fresh 3.5 cm AL plates and grown for 60 hr, before starting egg laying. Single worms were transferred to 3.5 cm NGM plates without peptone and without bacteria (starvation plates) for 2 hr to get rid of remaining OP50 bacteria. They were then transferred to fresh starvation plates and monitored for the timepoint of first egg laying. From 70 hr on, worms were monitored for death caused by internal hatching events (bagging/exploding phenotype) and for rarely occurring death events not caused by internal hatching. Worms that crawled off the agar were censored from the analysis. The assay was repeated three times.

Developmental assay. Developmental timing in wildtype (N2) and Cel-cest-1.2 mutant worms grown up under high density (HD) conditions was measured as previously described by determining the time point of first egg laying.¹⁶ Briefly, around 40 gravid young adults were allowed to lay eggs for 1 hr on NGM plates seeded with OP50 E. coli bacteria. 25 Single eggs were then transferred to a fresh plate. After 59 hr animals were scored for the timepoint of first egg laying using a Leica S6E stereo microscope.

¹³C₆-Leu isotope tracing experiment. Approximately 60,000 synchronized N₂ (wildtype C. elegans) and Cel-daf-22 mutant L1 larvae were seeded in 125 mL Erlenmeyer flasks containing 20 mL S-Complete medium. Worms were fed with 3 mg/mL freeze-dried OP50 powder (InVivoBiosystems, formerly NemaMetrix Inc., cat. #OP-50-31772) and supplemented with either L-Leucine (Sigma-Aldrich cat. #L8000) or ¹³C₆-L-Leucine (Cambridge Isotope Laboratories cat. #CLM-2262-H—PK) at a final concentration of 2 mM. Worms were incubated at 20° C. with shaking at 180 RPM for approx. 70 hr, at which time the population was a mixture of young and gravid adults, determined by microscopic inspection. Liquid cultures were centrifuged (500 g, 22° C., 1 min), and the resulting supernatant was snap frozen. Worm pellet was washed three times with M9 before snap freezing in liquid nitrogen. Frozen samples were lyophilized and extracted as above (Metabolite extraction).

It will appreciated that certain compounds of Tables S4a and S4b observed in C. elegans and C. briggsae have been chemically synthesized in order to confirm structural assignments. Such syntheses are described in the ensuing examples. The skilled person will recognize that individual compounds not explicity described synthetically below can be made using methods similar to those described, substituting appropriate starting materials or intermediates to arrive at the desired compound.

### B. Synthetic Procedures

General synthetic procedures. Unless noted otherwise, all chemicals and reagents were purchased from Sigma-Aldrich. All oxygen and moisture-sensitive reactions were carried out under argon atmosphere in flame-dried glassware. Solutions and solvents sensitive to moisture and oxygen were transferred via standard syringe and cannula techniques. All commercial reagents were purchased as reagent grade and, unless otherwise stated, were purchased from Sigma-Aldrich and used without any further purification. Boc-2-Abz-OH was purchased from Chem-impex. Acetic acid (AcOH), acetonitrile (ACN), dichloromethane (DCM), ethyl acetate (EtOAc), formic acid, hexanes and methanol (MeOH) used for chromatography and as a reagent or solvent were purchased from Fisher Scientific. Thin-layer chromatography (TLC) was performed using J. T. Baker Silica Gel IB2F plates. Flash chromatography was performed using Teledyne Isco CombiFlash systems and Teledyne Isco RediSep Rf silica and C18 columns. All deuterated solvents were purchased from Cambridge Isotopes. Nuclear Magnetic Resonance (NMR) spectra were recorded on Bruker INOVA 500 (500 MHz) and Varian INOVA 600 (600 MHz) spectrometers at Cornell University's NMR facility and Bruker AVANCE III HD 800 MHz (800 MHz) or Bruker AVANCE III HD 600 MHz (600 MHz) at SUNY ESF's NMR facility. ¹H NMR chemical shifts are reported in ppm (δ) relative to residual solvent peaks (7.26 ppm for chloroform-d, 3.31 ppm for methanol-d₄, 2.50 for DMSO-d₆). NMR-spectroscopic data are reported as follows: chemical shift, multiplicity (s=singlet, d=doublet, t=triplet, q=quartet, m=multiplet, br=broad), coupling constants (Hz), and integration and often tabulated including 2D NMR data. ¹³C NMR chemical shifts are reported in ppm (δ) relative to residual solvent peaks (77.16 ppm for chloroform-d, 49.00 ppm for methanol-d₄, 39.52 for DMSO-d₆). All NMR data processing was done using MNOVA 14.2.1 (mestrelab.com).

### Abbreviations

HPLC-HRMS, high performance liquid chromatography-high resolution mass spectrometry; MOGL, modular glucoside; MS/MS, tandem mass spectrometry; LRO, lysosome related organelle; UGT, uridine diphosphoglucuronosyltransferase; UDP, uridine 5′-diphosphate; CEST, carboxylesterase; ESI-, electrospray ionization negative mode; ESI+, electrospray ionization positive mode; mCPBA, 3-chloroperoxybenzoic acid.

iglu #1 (4) was synthesized as described previously.¹⁷

iglu #3 (10) was synthesized as described previously.²

Iglu #301 (31) was synthesized as described previously.²

### Example 1. Step 1

### (6aR,8R,9R,10R,10aS)-8-(1H-indol-1-yl)-2,2,4,4-tetraisopropylhexahydropyrano[3,2-f][1,3,5,2,4]trioxadisilocine-9,10-diol (S1)

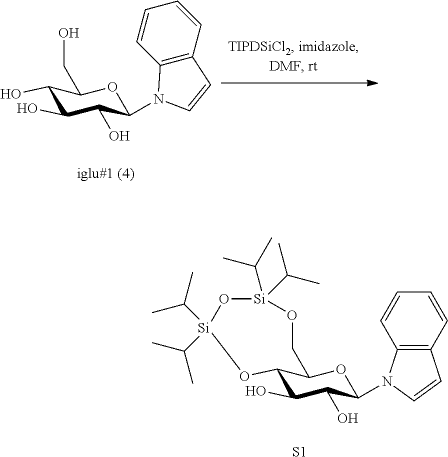

To 2.5 mL of DMF was added iglu #1 (4, 144.6 mg, 0.518 mmol, 1.0 equiv.) and imidazole (155.0 mg, 2.28 mmol, 4.4 equiv.). The stirred mixture was cooled to 0° C. before adding 1,3-dichloro-1,1,3,3-tetraisopropyldisiloxane (215 μL, 0.673 mmol, 1.3 equiv.). The reaction mixture was stirred at room temperature for 30 min, diluted with DCM, and then quenched with water. The organics were washed with sat. aq. NaHCO₃, dried with Na₂SO₄, and concentrated in vacuo. Flash column chromatography on silica using a gradient of 0-20% MeOH in DCM afforded S1 (250.5 mg, 93%) as an orange oil. ¹H NMR (500 MHz, chloroform-d): δ (ppm) 7.60 (d, J=7.8 Hz, 1H), 7.50 (d, J=8.2 Hz, 1H), 7.24 (d, J=3.5 Hz, 1H), 7.19 (ddd, J=1.2, 7.6, 8.6 Hz, 1H), 7.12 (ddd, J=0.9, 7.5, 8.0 Hz, 1H), 5.36 (d, J=8.9 Hz, 1H), 4.13 (dd, J=2.0, 12.7 Hz, 1H), 4.04 (t, J=7.7 Hz, 1H), 4.02 (t, J=8.2 Hz, 1H), 3.97 (dd, J=1.6, 12.7 Hz, 1H), 3.80 (t, J=9.0 Hz, 1H), 3.47 (dt, J=1.6, 9.0 Hz, 1H), 1.17-1.03 (m, 28H).

### Example 1. Step 2

### (6aR,8R,9R,10R,10aS)-10-hydroxy-8-(1H-indol-1-yl)-2,2,4,4-tetraisopropyl-hexahydropyrano[3,2-f][1,3,5,2,4]trioxadisilocin-9-yl benzoate (16)

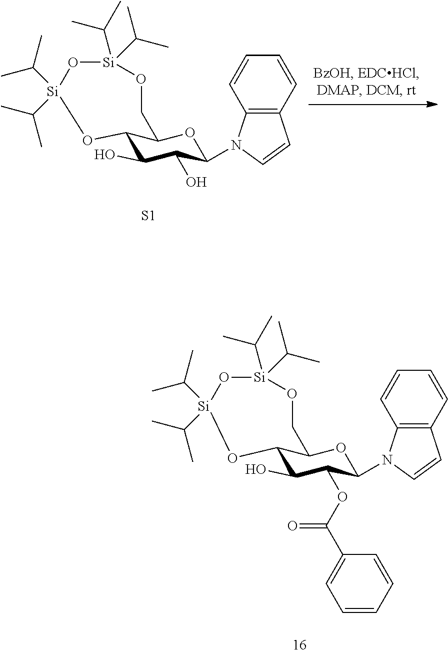

To a stirred solution of benzoic acid (14.4 mg, 0.118 mmol, 1.0 equiv.) in DCM, EDC·HCl (45.2 mg, 0.236 mmol, 2.0 equiv.) was added. The mixture was stirred at room temperature for 40 min, and S1 (73.8 mg, 0.142 mmol, 1.2 equiv.) and DMAP (36.0 mg, 0.295 mmol, 2.5 equiv.) were added. The reaction mixture was stirred at room temperature for 2.5 hours. The reaction mixture was concentrated in vacuo followed by flash column chromatography on silica using a gradient of 5-80% EtOAc in hexanes affording 16 (72.3 mg, 98%). ¹H NMR (500 MHz, chloroform-d): δ (ppm) 7.77 (dd, J=1.2, 8.5 Hz, 2H), 7.52 (d, J=7.8 Hz, 1H), 7.49-7.44 (m, 2H), 7.32-7.28 (m, 3H), 7.18 (ddd, J=1.0, 7.7, 8.3 Hz, 1H), 7.07 (ddd, J=0.8, 7.6, 8.3 Hz, 1H), 6.46 (d, J=3.4 Hz, 1H), 5.72 (d, J=9.2 Hz, 1H), 5.66 (t, J=9.0 Hz, 1H), 4.17 (dd, J=2.1, 12.6 Hz, 1H), 4.14 (t, J=9.0 Hz, 1H), 4.08 (t, J=9.0 Hz, 1H), 4.02 (dd, J=1.2, 12.6 Hz, 1H), 3.55 (dt, J=1.6, 9.2 Hz, 1H), 1.19-1.03 (m, 28H).

### Example 1. Step 3

### (6aR,8R,9R,10R,10aR)-10-((bis(benzyloxy)phosphoryl)oxy)-8-(1H-indol-1-yl)-2,2,4,4-tetraisopropylhexahydropyrano[3,2-f][1,3,5,2,4]trioxadisilocin-9-yl benzoate (19)

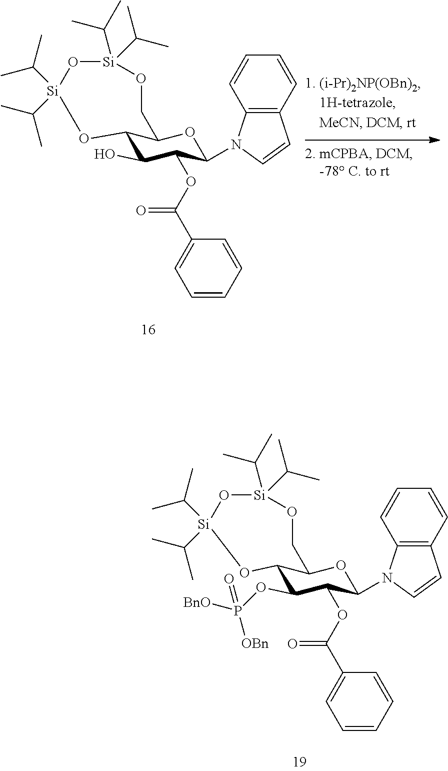

To a solution of 16 (32.5 mg, 0.052 mmol, 1.0 equiv.) in 0.8 mL DCM was added dibenzyl N,N-diisopropylphosphoramidite (105 μL, 0.312 mmol, 6.0 equiv.) and 1H-tetrazole (0.45 M in ACN, 693 μL, 0.312 mmol, 6.0 equiv.). The reaction mixture was stirred at room temperature for 30 min. Then the solution was cooled to −78° C. under argon before adding 3-chloroperoxybenzoic acid (mCPBA, ˜77%, 81.6 mg, 0.364 mmol, 7.0 equiv.). The solution was stirred at room temperature for 2 hr. The mixture was diluted with DCM and washed with sat. aq. NaHCO₃, dried with Na₂SO₄, and concentrated in vacuo. Flash column chromatography on silica using a gradient of 0-90% EtOAc in hexanes afforded 19 (32.2 mg, 70%) as a roughly 1:1 mixture with excess dibenzyl diisopropylphosphoramidite. ¹H NMR (500 MHz, chloroform-d): δ (ppm) 7.77 (dd, J=1.3, 8.3 Hz, 2H), 7.49 (d, J=7.7 Hz, 1H), 7.47-7.40 (m, 2H), 7.27 (m, 2H), 7.38-7.28 (m, 9H, with impurity), 7.21-7.14 (m, 3H, with impurity), 7.23 (d, J=3.4 Hz, 1H), 7.14 (m, 1H), 7.03 (ddd, J=0.8, 7.4, 8.3 Hz, 1H), 6.92 (m, 2H), 6.44 (d, J=3.4 Hz, 1H), 5.80 (t, J=9.1 Hz, 1H), 5.66 (d, J=9.1 Hz, 1H), 4.92 (d, J=9.1 Hz, 1H), 4.86 (dd, J=6.8, 11.7 Hz, 1H), 4.78-4.69 (m, 2H), 4.51 (dd, J=9.7, 11.8 Hz, 1H), 4.32 (t, J=9.3 Hz, 1H), 4.19 (dd, J=1.9, 12.7 Hz, 1H), 4.03 (dd, J=1.2, 12.7 Hz, 1H), 1.18 (d, J=7.1 Hz, 3H), 1.17 (d, J=7.8 Hz, 3H), 1.10-0.99 (m, 19H), 0.94 (d, J=6.9 Hz, 3H).

### Example 1. Step 4

### (2R,3R,4S,5R,6R)-4-((bis(benzyloxy)phosphoryl)oxy)-5-hydroxy-6-(hydroxymethyl)-2-(1H-indol-1-yl)tetrahydro-2H-pyran-3-yl benzoate (22)

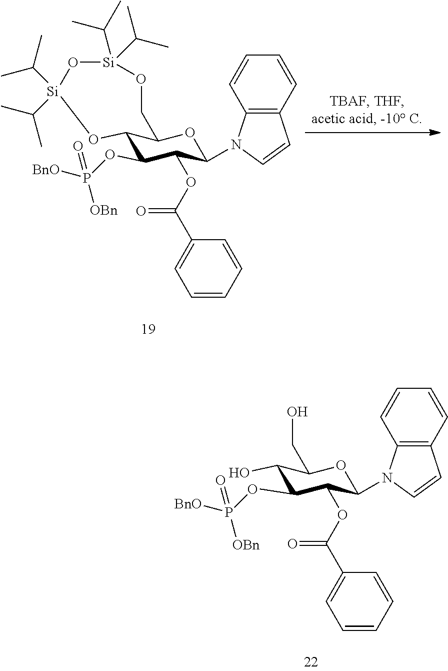

To a solution of 19 (32.2 mg, 0.0364 mmol, 1.0 equiv.) in 1 mL THF was added acetic acid (6 μL, 0.109 mmol, 3.0 equiv.), and the mixture was cooled to −10° C. Tetrabutylammonium fluoride (1M in THF, 109 μL, 0.109 mmol, 3.0 eq) was added, and the solution was stirred for 10 min. Subsequently, acetic acid (15 μL, 0.262 mmol, 7.2 equiv.) was added, and the reaction mixture was concentrated in vacuo. Flash column chromatography on silica using a gradient of 0-40% MeOH in DCM afforded 22 (23.1 mg, 99%). Product contained 45% of impurity dibenzyl diisopropylphosphoramidite. ¹H NMR (500 MHz, methanol-d₄): δ (ppm) 7.70 (dd, J=1.2, 8.3 Hz, 2H), 7.60 (d, J=8.4 Hz, 1H), 7.43 (m, 1H), 7.41 (br, 1H), 7.24 (m, 2H), 7.10 (m, 1H), 6.97 (m, 1H), 6.94 (m, 2H), 6.40 (d, J=3.3 Hz, 1H), 6.03 (d, J=9.2 Hz, 1H), 6.44 (t, J=9.2 Hz, 1H), 5.07-4.92 (m, 5H), 4.00-3.95 (m, 2H), 3.88-3.90 (m, 2H).

### Example 1. Step 5

### (2R,3R,4S,5R,6R)-5-hydroxy-6-(hydroxymethyl)-2-(1H-indol-1-yl)-4-(phosphonooxy)tetrahydro-2H-pyran-3-yl benzoate (iglu #121, 25)

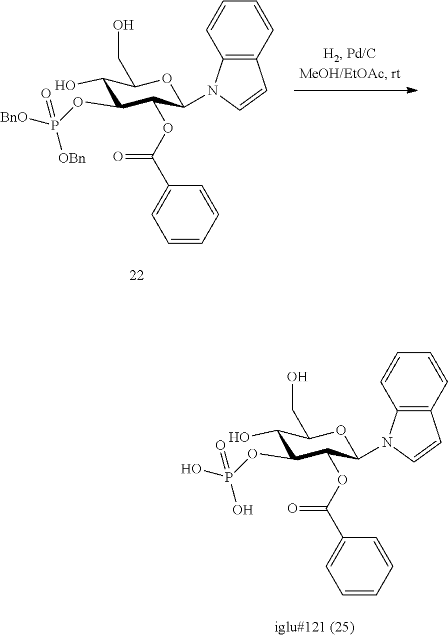

To a mixture of 1:1 MeOH/EtOAc (v/v, 2 mL) and 22 (23.1 mg, 0.0359 mmol, 1.0 equiv.) was added Pd/C (10% w/w) (20 mg). The reaction mixture was purged with argon for 2 min, then H₂ gas was bubbled through for 45 min at room temperature, and the reaction vessel was again purged with argon for 2 min. The reaction mixture was filtered through Celite and concentrated in vacuo. The crude mixture was purified by reversed-phase flash chromatography with a C18 column using a gradient of 0-60% ACN in H₂O (with 0.1% formic acid), which afforded iglu #121 (25, 2.4 mg, 14%) as clear oil. See Table S1 for NMR spectroscopic data of iglu #121 (25).

### Example 2. Step 1

### (6aR,8R,9R,10R,10aS)-10-hydroxy-8-(1H-indol-1-yl)-2,2,4,4-tetraisopropyl-hexahydropyrano[3,2-f][1,3,5,2,4]trioxadisilocin-9-yl 1H-pyrrole-2-carboxylate (17)

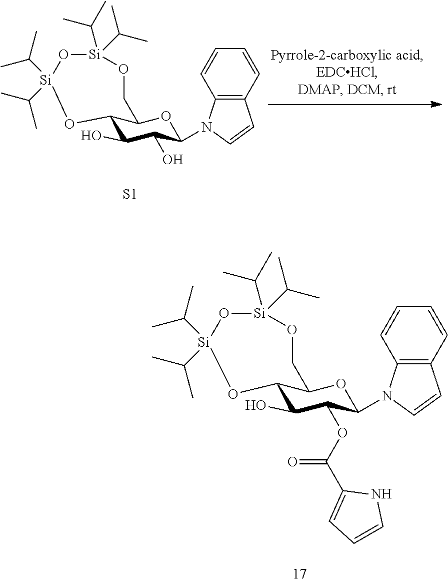

To a stirred solution of pyrrole-2-carboxylic acid (13.4 mg, 0.121 mmol, 1.0 equiv.) in DCM, EDC·HCl (46.0 mg, 0.240 mmol, 2.0 equiv.) was added. The mixture was stirred at room temperature for 30 min, and S1 (75.2 mg, 0.144 mmol, 1.2 equiv.) and DMAP (36.7 mg, 0.30 mmol, 2.5 equiv.) were added. The reaction mixture was stirred at room temperature for 4 hours. The reaction mixture was concentrated in vacuo followed by flash column chromatography on silica using a gradient of 0-80% EtOAc in hexanes, affording 17 (38.7 mg, 44%). ¹H NMR (500 MHz, chloroform-d): δ (ppm) 8.92 (s, 1H), 7.52 (d, J=7.8 Hz, 1H), 7.47 (d, J=8.3 Hz, 1H), 7.26 (d, J=3.4 Hz, 1H), 7.19 (ddd, J=1.0, 7.2, 9.3 Hz, 1H), 7.08 (ddd, J=0.8, 7.0, 8.6 Hz, 1H), 6.76 (m, 1H), 6.72 (m, 1H), 6.45 (d, J=3.4 Hz, 1H), 6.11 (m, 1H), 5.65 (d, J=9.2 Hz, 1H), 5.49 (t, J=9.2 Hz, 1H), 4.15 (dd, J=2.0, 12.7 Hz, 1H), 4.09 (dd, J=3.4, 8.7 Hz, 1H), 4.03-3.98 (m, 2H), 3.52 (dt, J=1.6, 9.2 Hz, 1H), 1.17-1.01 (m, 28H).

### Example 2. Step 2

### (6aR,8R,9R,10R,1aR)-10-((bis(benzyloxy)phosphoryl)oxy)-8-(1H-indol-1-yl)-2,2,4,4-tetraisopropylhexahydropyrano[3,2-f][1,3,5,2,4]trioxadisilocin-9-yl 1H-pyrrole-2-carboxylate (20)

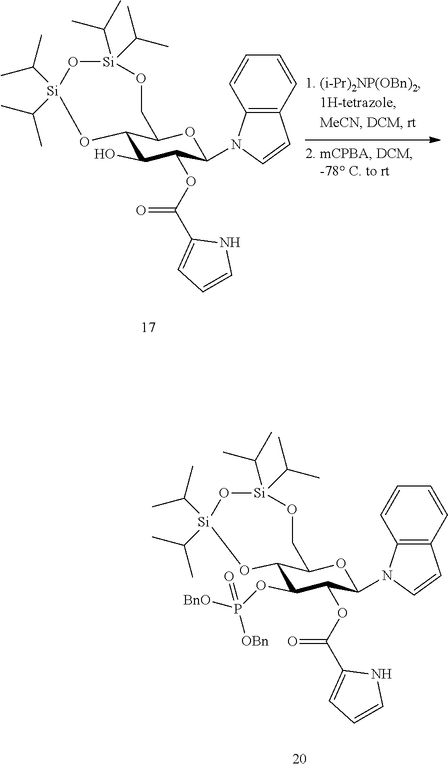

To a solution of 17 (38.7 mg, 0.063 mmol, 1.0 equiv.) in 1 mL DCM was added dibenzyl N,N-diisopropylphosphoramidite (64 μL, 0.189 mmol, 3.0 equiv.) and 1H-tetrazole (0.45 M in ACN, 420 μL, 0.189 mmol, 3.0 equiv.). The reaction mixture was stirred at room temperature for 30 min. Then the solution was cooled to −78° C. under argon before added 3-chloroperoxybenzoic acid (mCPBA, ˜77%, 44.0 mg, 0.196 mmol, 3.1 equiv.). The solution was stirred to up room temperature over a 2-hr period. The mixture was diluted with DCM and washed with sat. aq. NaHCO₃, dried with Na₂SO₄, and concentrated in vacuo. Flash column chromatography on silica using a gradient of 0-80% EtOAc in hexanes afforded 20 (47.8 mg, 87%). ¹H NMR (500 MHz, chloroform-d): δ (ppm) 9.68 (s, 1H), 7.53 (dt, J=1.0, 7.7 Hz, 1H), 7.45 (d, J=8.3 Hz, 1H), 7.37-7.32 (m, 3H), 7.27 (d, J=3.4 Hz, 1H), 7.24-7.20 (m, 4H), 7.13 (ddd, J=1.1, 6.9, 7.9 Hz, 1H), 7.02 (ddd, J=0.9, 6.9, 7.9 Hz, 1H), 6.92 (dd, J=1.4, 7.7 Hz, 1H), 6.78 (m, 1H), 6.48 (d, J=3.4 Hz, 1H), 6.11 (m, 1H), 5.63 (t, J=9.0 Hz, 1H), 5.58 (d, J=8.9 Hz, 1H), 5.02 (m, 1H), 4.97 (dd, J=4.8, 12.0 Hz, 1H), 4.95-4.88 (m, 2H), 4.68 (dd, J=7.2, 11.7 Hz, 1H), 4.53 (dd, J=8.4, 11.7 Hz, 1H), 4.29 (t, J=9.4 Hz, 1H), 4.17 (dd, J=1.9, 12.7 Hz, 1H), 4.01 (dd, J=1.2, 12.7 Hz, 1H), 3.50 (m, 1H), 1.24 (d, J=6.7 Hz, 3H), 1.18 (d, J=6.9 Hz, 3H), 1.11-0.93 (m, 22H).

### Example 2. Step 3

### (2R,3R,4S,5R,6R)-4-((bis(benzyloxy)phosphoryl)oxy)-5-hydroxy-6-(hydroxymethyl)-2-(1H-indol-1-yl)tetrahydro-2H-pyran-3-yl 1H-pyrrole-2-carboxylate (23)

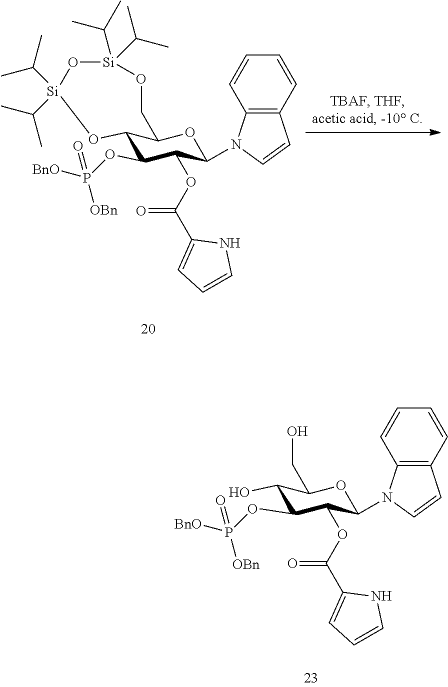

To a solution of 20 (47.8 mg, 0.0547 mmol, 1.0 equiv.) in 1 mL THF was added acetic acid (9.4 μL, 0.164 mmol, 3.0 equiv.) and cooled to −10° C. The solution was added tetrabutylammonium fluoride (1M in THF, 164 μL, 0.164 mmol, 3.0 equiv.) and stirred for 1.5 hr. The reaction mixture was added acetic acid (10 μL, 0.175 mmol, 3.2 equiv.) and concentrated in vacuo. Flash column chromatography on silica using a gradient of 0-40% MeOH in DCM afforded 23 (28.4 mg, 82%). ¹H NMR (500 MHz, methanol-d₄): δ (ppm) 7.59 (d, J=8.4 Hz, 1H), 7.44 (d, J=7.8 Hz, 1H), 7.40 (d, J=3.4 Hz, 1H), 7.31-7.19 (m, 7H), 7.12 (ddd, J=0.9, 7.1, 8.1 Hz, 1H), 6.98 (m, 2H), 6.85 (m, 1H), 6.73 (dd, J=1.5, 3.9 Hz, 1H), 6.41 (d, J=3.4 Hz, 1H), 6.06 (dd, J=2.5, 3.7 Hz, 1H), 5.94 (d, J=9.2 Hz, 1H), 5.77 (t, J=9.3 Hz, 1H), 5.06 (dd, J=7.3, 11.8 Hz, 1H), 4.97 (dd, J=8.3, 11.8 Hz, 1H), 4.89 (q, J=8.9 Hz, 1H), 4.76 (dd, J=7.3, 11.8 Hz, 1H), 4.63 (dd, J=8.3, 11.8 Hz, 1H), 3.98-3.91 (m, 2H), 3.82 (dd, J=5.4, 12.0 Hz, 1H), 3.78 (ddd, J=1.8, 5.3, 9.7 Hz, 1H).

### Example 2. Step 4

### (2R,3R,4S,5R,6R)-5-hydroxy-6-(hydroxymethyl)-2-(1H-indol-1-yl)-4-(phosphonooxy)tetrahydro-2H-pyran-3-yl 1H-pyrrole-2-carboxylate (26)

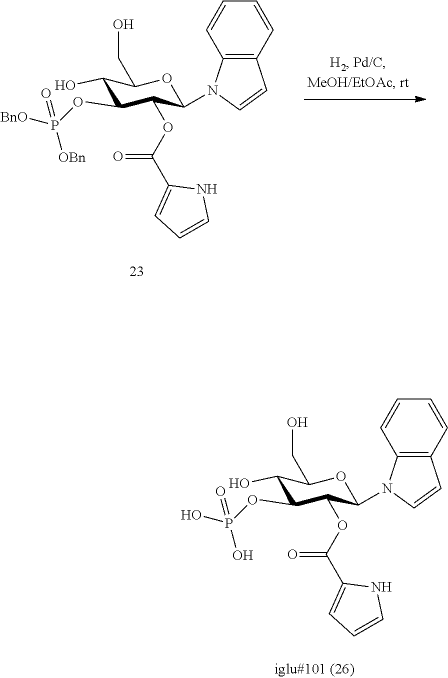

To a 1:1 mixture of MeOH/EtOAc (v/v, 2 mL) 23 (28.4 mg, 0.0449 mmol, 1.0 equiv.) and Pd/C (10% w/w) (23 mg) were added. The reaction mixture was purged with argon for 2 min, subjected to H₂ for 1 hr, at room temperature, and again purged with argon for 2 min. The reaction mixture was filtered through Celite and concentrated in vacuo. The residue was purified by reversed-phase flash chromatography with a C18 column using a gradient of 0-60% ACN in H₂O (with 0.1% formic acid), which afforded iglu #101 (26, 9.2 mg, 45%) as a clear oil. See Table S3 for NMR spectroscopic data of iglu #101 (26).

### Example 3. Step 1

### (6aR,8R,9R,10R,10aS)-10-hydroxy-8-(1H-indol-1-yl)-2,2,4,4-tetraisopropyl-hexahydropyrano[3,2-f][1,3,5,2,4]trioxadisilocin-9-yl 2-((tert-butoxycarbonyl)-amino)benzoate (18)

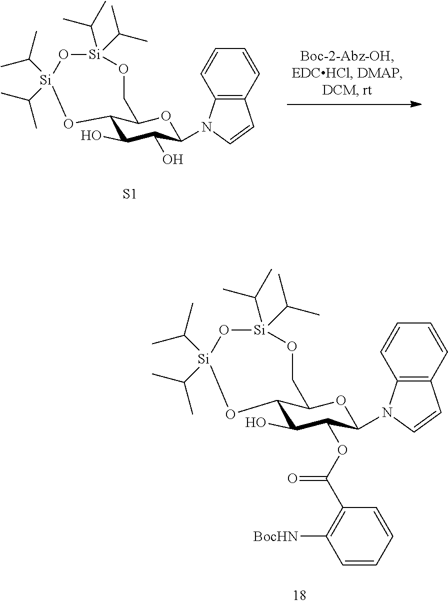

To a stirred solution of Boc-2-Abz-OH (24.0 mg, 0.101 mmol, 1.0 equiv.) in DCM, EDC·HCl (38.7 mg, 0.202 mmol, 2.0 equiv.) was added. The mixture was stirred at room temperature for 30 min, and S1 (63.0 mg, 0.121 mmol, 1.2 equiv.) and DMAP (30.8 mg, 0.252 mmol, 2.5 equiv.) were added. The reaction mixture was stirred at room temperature for 4 hr. The reaction mixture was concentrated in vacuo followed by flash column chromatography on silica using a gradient of 5-90% EtOAc in hexanes, which afforded 18 (21.6 mg, 29%). ¹H NMR (500 MHz, chloroform-d): δ (ppm) 9.76 (s, 1H), 8.33 (dd, J=0.8, 8.6 Hz, 1H), 7.69 (dd, J=1.6, 8.2 Hz, 1H), 7.47 (d, J=8.3 Hz, 1H), 7.41 (ddd, J=1.3, 1.6, 7.9 Hz, 1H), 7.25 (d, 1H), 7.18 (ddd, J=1.0, 1.0, 7.7 Hz, 1H), 7.07 (ddd, J=0.8, 0.8, 7.5 Hz, 1H), 6.84 (ddd, J=1.0, 1.0, 7.7 Hz, 1H), 6.47 (d, J=3.4 Hz, 1H), 5.71-5.64 (m, 2H), 4.16 (dd, J=1.9, 12.6 Hz, 1H), 4.14-4.09 (m, 2H), 4.03 (dd, J=1.1, 12.6 Hz, 1H), 3.55 (m, 1H), 1.49 (s, 9H), 1.18-1.02 (m, 28H).

### Example 3. Step 2

### (6aR,8R,9R,10R,10aR)-10-((bis(benzyloxy)phosphoryl)oxy)-8-(1H-indol-1-yl)-2,2,4,4-tetraisopropylhexahydropyrano[3,2-f][1,3,5,2,4]trioxadisilocin-9-yl 2-((tert-butoxycarbonyl)amino)benzoate (21)

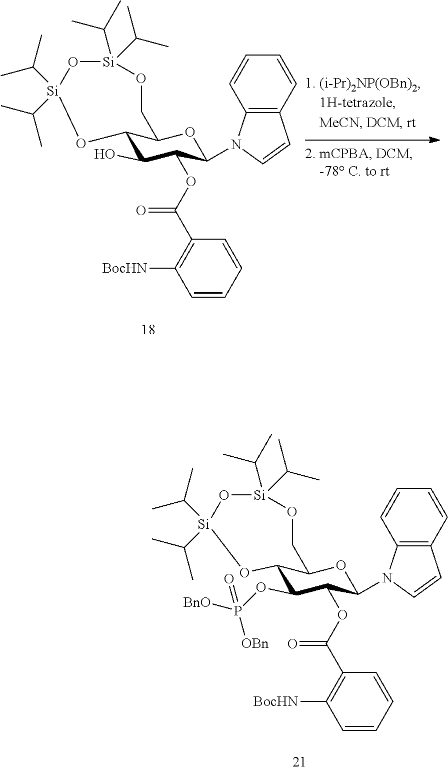

To a solution of 18 (21.6 mg, 0.0292 mmol, 1.0 equiv.) in 0.8 mL DCM was added dibenzyl N,N-diisopropylphosphoramidite (29 μL, 0.0875 mmol, 3.0 equiv.) and 1H-tetrazole (0.45 M in ACN, 194 μL, 0.0875 mmol, 3.0 equiv.). The reaction mixture was stirred at room temperature for 45 min. Then the solution was cooled to −78° C. under argon before adding 3-chloroperoxybenzoic acid (mCPBA, ˜77%, 20 mg, 0.364 mmol, 3.0 equiv.). The solution was stirred at room temperature for 2-hr. The mixture was diluted with DCM and washed with sat. aq. NaHCO₃, dried with Na₂SO₄, and concentrated in vacuo. Flash column chromatography on silica using a gradient of 5-90% EtOAc in hexanes afforded 21 (25.5 mg, 87%). ¹H NMR (500 MHz, chloroform-d): δ (ppm) 9.48 (s, 1H), 8.25 (d, J=8.4 Hz, 1H), 7.85 (dd, J=1.1, 8.0 Hz, 1H), 7.50 (d, J=8.0 Hz, 1H), 7.48 (d, J=8.5 Hz, 1H), 7.39 (m, 1H), 7.36-7.33 (m, 2H), 7.25-7.09 (m, 8H), 7.05 (ddd, J=0.7, 7.6, 7.8 Hz, 1H), 6.95 (m, 2H), 6.88 (ddd, J=0.7, 7.6, 8.4 Hz, 1H), 6.43 (d, J=3.3 Hz, 1H), 5.75 (d, J=9.1 Hz, 1H), 5.61 (d, J=9.0 Hz, 1H), 4.90-4.82 (m, 2H), 4.77 (dd, J=7.2, 11.6 Hz, 1H), 4.69 (dd, J=8.3, 11.6 Hz, 1H), 4.46 (t, J=11.2 Hz, 1H), 4.31 (t, J=9.2 Hz, 1H), 4.20 (dd, J=1.3, 12.7 Hz, 1H), 4.05 (dd, J=1.0, 12.7 Hz, 1H), 3.53 (d, J=9.3 Hz, 1H), 1.41 (s, 9H), 1.32-1.26 (m, 7H), 1.19 (d, J=6.8 Hz, 3H), 1.17 (d, J=6.8 Hz, 3H), 1.04-1.00 (m, 6H), 0.95 (d, J=6.8 Hz, 3H), 0.89 (d, J=6.8 Hz, 3H), 0.88 (d, J=7.1 Hz, 3H). ¹³C NMR (125 MHz, chloroform-d): 171.3, 167.3, 152.7, 142.6, 136.4, 135.5, 135.4, 135.0, 134.97, 134.92, 131.4, 129.3, 128.3, 128.2, 128.1, 127.41, 127.38, 125.2, 122.6, 121.4, 121.2, 120.8, 118.5, 113.9, 109.8, 104.4, 80.4, 79.9, 69.38, 69.34, 68.13, 68.09, 68.06, 59.2, 31.7, 28.4, 22.8, 21.2, 18.3, 17.43, 17.42, 17.39, 17.2, 17.0, 14.3, 14.2, 13.5, 13.3, 12.9, 12.6.

### Example 3. Step 3

### (2R,3R,4S,5R,6R)-4-((bis(benzyloxy)phosphoryl)oxy)-5-hydroxy-6-(hydroxymethyl)-2-(1H-indol-1-yl)tetrahydro-2H-pyran-3-yl 2-((tert-butoxycarbonyl)amino)benzoate (24)

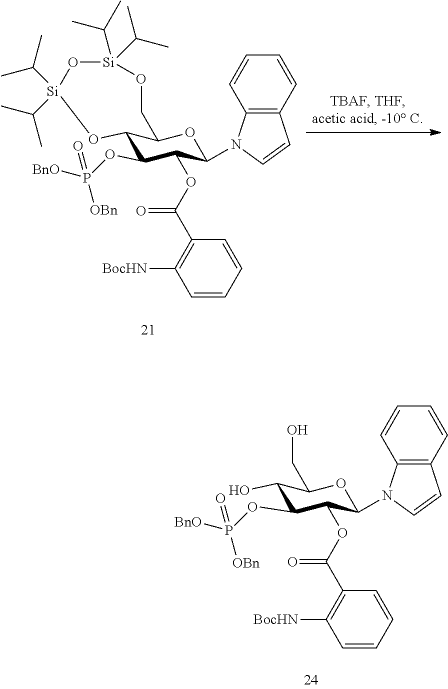

To a solution of 21 (25.5 mg, 0.0255 mmol, 1.0 equiv.) in 1 mL THF was added acetic acid (4.4 μL, 0.0765 mmol, 3.0 equiv.), and the mixture was cooled to −10° C. To the solution was added tetrabutylammonium fluoride (1M in THF, 77 μL, 0.0765 mmol, 3.0 eq) and the resulting mixture stirred for 1.4 hr. Subsequently, acetic acid (10 μL, 0.175 mmol, 6.8 equiv.) was added and the mixture concentrated in vacuo. Flash column chromatography on silica using a gradient of 5-20% MeOH in DCM afforded 24 (17.1 mg, 88%), containing about 20% of dibenzyl diiylphosphoramidite as an impurity. ¹H NMR (600 MHz, chloroform-d): δ (ppm) 9.64 (s, 1H), 8.33 (dd, J=1.1, 8.6 Hz, 1H), 7.71 (dd, J=1.6, 8.1 Hz, 1H), 7.54 (dt, J=0.9, 7.9 Hz, 1H), 7.43-7.39 (m, 2H), 7.36-7.34 (m, 2H), 7.30 (d, J=2.2 Hz, 1H), 7.30-7.27 (m, 2H), 7.24 (m, 1H), 7.21 (m, 1H), 7.14 (m, 2H), 7.10 (ddd, J=0.9, 7.5, 8.0 Hz, 1H), 7.03 (m, 2H), 6.80 (dt, J=1.1, 7.6 Hz, 1H), 6.51 (d, J=3.4 Hz, 1H), 5.80 (t, J=9.3 Hz, 1H), 5.68 (d, J=9.3 Hz, 1H), 4.96 (dd, J=8.2, 11.7 Hz, 1H), 4.83 (dd, J=7.9, 11.7 Hz, 1H), 4.80 (d, J=8.7 Hz, 1H), 4.68 (dt, J=7.2, 9.0 Hz, 1H), 4.02-3.94 (m, 2H), 3.88 (dd, J=5.1, 12.1 Hz, 1H), 3.72 (ddd, J=3.3, 5.1, 9.6 Hz, 1H), 1.44 (s, 9H). ¹³C NMR (125 MHz, chloroform-d): 175.4, 170.0, 152.5, 142.4, 136.3, 135.24, 135.19, 135.0, 134.93, 134.87, 130.7, 129.2, 128.69, 128.68, 128.62, 128.58, 127.8, 124.7, 122.5, 121.3, 121.1, 120.8, 118.6, 112.9, 109.8, 104.4, 83.1, 82.3, 82.2, 80.7, 78.6, 70.85, 70.81, 70.16, 70.11, 70.08, 70.04, 69.79, 69.78, 61.9, 50.6, 28.3.

### Example 3. Step 4

### (2R,3R,4S,5R,6R)-4-((bis(benzyloxy)phosphoryl)oxy)-5-hydroxy-6-(hydroxymethyl)-2-(1H-indol-1-yl)tetrahydro-2H-pyran-3-yl 2-aminobenzoate (27)

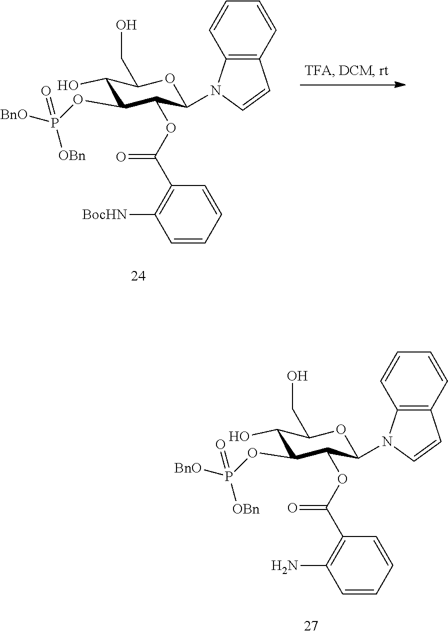

To a solution of 24 (17.1 mg, 0.0226 mmol, 1.0 equiv.) in 1.5 mL DCM was added TFA (0.1 mL, 1.31 mmol, 58 equiv.). The reaction mixture was stirred at room temperature for 20 min and then concentrated in vacuo. Flash column chromatography on silica using a gradient of 0-20% MeOH in DCM afforded 27 (14.7 mg, 99%), containing 27% of dibenzyl diisopropylphosphoramidite as impurity. ¹H NMR (600 MHz, DMSO-d₄): δ (ppm) 7.72 (d, J=8.4 Hz, 1H), 7.61 (dd, J=1.4, 8.2 Hz, 1H), 7.48 (d, J=3.4 Hz, 1H), 7.45 (d, J=7.8 Hz, 1H), 7.39-7.19 (m, 14H, with impurity), 7.13 (dt, J=1.7, 7.1 Hz, 1H), 7.00 (t, J=7.6 Hz, 1H), 6.97 (m, 2H), 6.61 (d, J=8.4 Hz, 1H), 6.43 (d, J=3.3 Hz, 1H), 6.37 (dt, J=1.0, 7.5 Hz, 1H), 6.20 (d, J=9.2 Hz, 1H), 5.90 (br, 1H), 5.76 (d, J=9.2 Hz, 1H), 4.94 (m, 2H), 4.70 (dd, J=7.1, 12.0 Hz, 1H), 4.56 (dd, J=8.0, 12.0 Hz, 1H), 3.85-3.73 (m, 3H), 3.60 (dd, J=5.6, 12.3 Hz, 1H).

### Example 3. Step 5

### (2R,3R,4S,5R,6R)-5-hydroxy-6-(hydroxymethyl)-2-(1H-indol-1-yl)-4-(phosphonooxy)tetrahydro-2H-pyran-3-yl 2-aminobenzoate (iglu #401, 28)

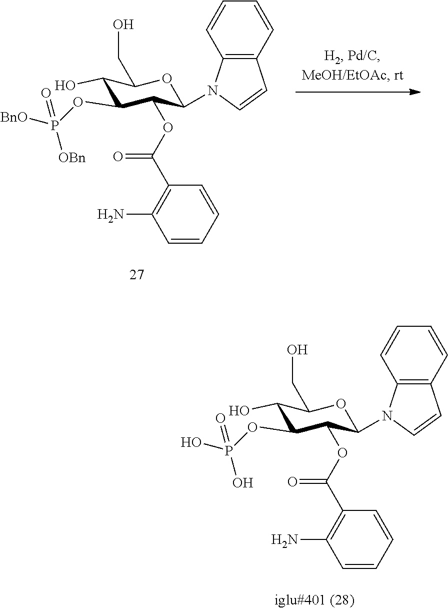

To a 1:1 mixture of MeOH/EtOAc (v/v, 2 mL) 27 (14.7 mg, 0.0223 mmol, 1.0 equiv.) and Pd/C (10% w/w) (14 mg) were added. The reaction mixture was purged with argon for 2 min, subjected to H₂ for 1 hr at room temperature, and again purged with argon for 2 min. The reaction mixture was filtered through Celite and concentrated in vacuo. The residue was purified by reversed-phase flash chromatography with a C18 column using a gradient of 0-60% ACN in H₂O (with 0.1% formic acid), which afforded iglu #401 (28, 1.6 mg, 15%) as a clear oil. See Table S2 for NMR spectroscopic data of iglu #401 (28).

### Synthesis of Selected Neurotransmitter-Derived MOGLs

### Reagents and General Procedures

All oxygen and moisture-sensitive reactions were carried out under argon atmosphere in flame-dried glassware. Solutions and solvents sensitive to moisture and oxygen were transferred via standard syringe and cannula techniques. Trimethylsilyl trifluoromethanesulfonate (TMSOTf) was transferred to a Schlenk flask prior to use and stored at −20° C. Methanolic ammonia (7N) was purchased from Acros Organics. All commercial reagents were purchased as reagent grade and, unless otherwise stated, were purchased from Sigma-Aldrich and used without any further purification. Acetic acid (AcOH), acetonitrile (ACN), dichloromethane (DCM), ethylacetate (EtOAc), N,N-dimethylformamide (DMF), tetrahydrofuran (THF), dimethylsulfoxide (DMSO), formic acid, hexanes, and methanol (MeOH) used for chromatography and as a reagent or solvent were purchased from ThermoFisher Scientific. Acetyl chloride (1-¹³C, 99%) was purchased from Cambridge Isotope Laboratories, N-acetylserotonin (NAS) was obtained from Biosynth International, Boc-2-aminobenzoic acid (Boc-2-Abz-OH) was from Chem-Impex International, and trifluoroacetic acid (TFA) was from Tokyo Chemical Industry, fluoxetine hydrochloride was from Spectrum Chemical. Dichloromethane (DCM), and N,N-dimethylformamide (DMF) were dried with 3 Å molecular sieves prior to use. Thin-layer chromatography (TLC) was performed using J. T. Baker Silica Gel IB2F plates. Flash chromatography was performed using Teledyne IscoCombiFlash systems and Teledyne Isco RediSep Rf silica and C₁₈ reverse phase columns. All deuterated solvents were purchased from Cambridge Isotopes. Abbreviations used: triethylamine (TEA), 2,3-dichloro-5,6-dicyano-1,4-benzoquinone (DDQ), trichloroacetonitrile (CCl₃CN), 1,8-diazabicyclo[5.4.0]undec-7-ene (DBU), trifluoromethanesulfonate (TMSOTf), N-ethyl-N′-(3-dimethylaminopropyl)carbodiimide hydrochloride (EDC·HCl), 4-dimethylaminopyridine (DMAP), 1,3-dichloro-1,1,3,3-tetraisopropyldisiloxane(TIPDSiCl₂), 3-chloroperoxybenzoic acid (m-CPBA). Nuclear Magnetic Resonance (NMR) spectra were recorded on Bruker INOVA 500 (500 MHz) and Varian INOVA 600 (600 MHz) spectrometers at Cornell University's NMR facility and Bruker AVANCE III HD 800 MHz (800 MHz) or Bruker AVANCE III HD 600 MHz (600 MHz) at SUNY ESF's NMR facility. ¹H NMR chemical shifts arereported in ppm (δ) relative to residual solvent peaks (7.26 ppm for chloroform-d, 3.31 ppm for methanol-d₄, 2.05 ppm for acetone-d₆). NMR-spectroscopic data are reported as follows: chemical shift, multiplicity (s=singlet, d=doublet, t=triplet, q=quartet, m=multiplet, br=broad), coupling constants (Hz), and integration and often tabulated including 2D NMR data. ¹³CNMR chemical shifts are reported in ppm (δ) relative to residual solvent peaks (77.16 ppm for chloroform-d, 49.00 ppm for methanol-d₄, 29.9 ppm for acetone-d₆). All NMR data processing was done using MestreLab MNOVA version 14.2.1-27684 (mestrelab.com).

### High-Performance Liquid Chromatography-Mass Spectrometry (HPLC-MS)

Several methods for chromatographic separation were utilized due to varying polarity of metabolites of interest. High resolution LC-MS analysis was performed on a Thermo Fisher Scientific Vanquish Horizon UHPLC System coupled with a Thermo Q Exactive HF hybrid quadropole-orbitrap high resolution mass spectrometer quipped with a HESI ion source. 1 μL of synthetic and natural endo- and exo-metabolome extracts (C. elegans N₂, C. briggsae AF-16, C. elegans him-5, and C. elegans fem-3 (gf)) were injected and separated according to the methods provided below:

Method A—water-acetonitrile gradient on a Hypersil GOLD C18 column (150 mm×2.1 mm 1.9 um particle size 175 Å pore size, Thermo Scientific) and maintained at 40° C. Solvent A: 0.1% formic acid in water; solvent B: 0.1% formic acid in acetonitrile. A/B gradient started at 1% B for 3 min, then from 1% to 99% B over 17 min, 99% B for 5 min, then rapidly down to 1% B over 0.5 min and held for 2.5 min to equilibrate the column.

Method B—water-acetonitrile gradient on a Hypersil GOLD C18 column (150 mm×2.1 mm 1.9 um particle size 175 Å pore size, Thermo Scientific) and maintained at 40° C. Solvent A: 0.1% formic acid in water; solvent B: 0.1% formic acid in acetonitrile. A/B gradient started at 1% B for 3 min, then from 1% to 35% B over 37 min, then from 35% to 100% B over 15 min, held at 100% B for 2 min, then rapidly down to 1% B over 0.5 min, and held for 2.5 min to equilibrate the column.

Method C—water-acetonitrile gradient on a Zorbax HILIC Plus column (150 mm×2.1 mm 1.8 um particle size 95 Å pore size, Agilent) and maintained at 40° C. Solvent A: 0.1% formic acid in water; solvent B: 0.1% formic acid in acetonitrile. A/B gradient started at 95% B for 4 min, then from 95% to 55% B over 15 min, then rapidly down to 5% B and held for 3 min, then back to 95% B and equilibrated for 3 min.

Method D—water-acetonitrile gradient on a XBridge Amide column (150 mm×2.1 mm 3.5 um particle size 130 Å pore size, Waters) and maintained at 40° C. Solvent A: 90% acetonitrile and 10% water prepared with 0.4% (v/v) of 25% ammonia in water solution combined with 0.1% (v/v) formic acid, solvent B: 30% acetonitrile and 70% water prepared with 0.4% (v/v) of 25% ammonia in water solution combined with 0.1% (v/v) formic acid. A/B gradient started at 1% B for 3 min, then from 1% to 60% B over 17 min, then from 60% to 100% B over 6 min and held for 1.5 min, then back to 1% B over 0.5 min and equilibrated for 2 min.

Method E—water-acetonitrile gradient on a XBridge Amide column (150 mm×2.1 mm 3.5 um particle size 130 Å pore size, Waters) and maintained at 40° C. Solvent A: 90% acetonitrile and 10% water prepared with 0.4% (v/v) of 25% ammonia in water solution combined with 0.1% (v/v) formic acid, solvent B: 30% acetonitrile and 70% water prepared with 0.4% (v/v) of 25% ammonia in water solution combined with 0.1% (v/v) formic acid. A/B gradient started at 1% B for 3 min, then from 1% to 35% B over 37 min, then from 35% to 100% B over 15 min and held for 2 min, then back to 1% B over 0.5 min and equilibrated for 2.5 min.

Mass spectrometer parameters: 3.5 kV spray voltage, 380° C. capillary temperature, 300° C. probe heater temperature, 60 sheath flow rate, 20 auxiliary flow 15 rate, 1 spare gas; S-lens RF level 50.0, resolution 240,000, m/z range 100-1200 m/z, AGC target 3e6. Instrument was calibrated with positive and negative ion calibration solutions (Thermo-Fisher) Pierce LTQ Velos ESI pos/neg calibration solutions. Peak areas were determined using Xcalibur 2.3 QualBrowser version 2.3.26 (Thermo Scientific) using a 5-10 ppm window around the m/z of interest.

### Example 4

### 4-((2-(5-hydroxy-1H-indol-3-yl)ethyl)amino)-4-oxobutanoic acid (14)

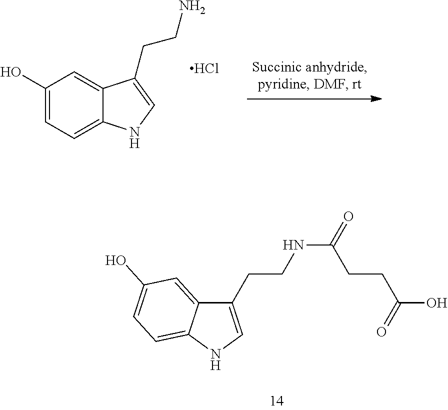 [SMILES: NCCc1c[nH]c2ccc(O)cc12 ; Cl ; O=C(O)CCC(=O)NCCc1c[nH]c2ccc(O)cc12]

To a solution of serotonin hydrochloride (128.1 mg, 0.602 mmol, 1.0 equiv.) in DMF (6 mL) was added succinic anhydride (78.3 mg, 0.783 mmol, 1.3 equiv.) and pyridine (0.6 mL). The mixture was stirred at room temperature for 24 hours and concentrated in vacuo. Flash column chromatography on silica using a gradient of 0-50% MeOH in DCM afforded 14 (165.4 mg, 99%) as clear oil. 1H NMR (500 MHz, methanol-d₄): δ (ppm) 7.16 (d, J=8.6 Hz, 1H), 6.98 (s, 1H), 6.96 (d, J=2.3 Hz, 1H), 6.69 (dd, J=2.3, 8.6 Hz, 1H), 3.42 (t, J=7.2 Hz, 2H), 2.83 (t, J=7.2 Hz, 2H), 2.57 (t, J=7.0 Hz, 2H), 2.43 (t, J=7.0 Hz, 2H). ¹³C NMR (125 MHz, methanol-d₄): δ (ppm) 176.3, 174.3, 151.0, 132.9, 129.3, 124.3, 112.7, 112.4, 112.3, 103.5, 41.3, 31.5. 30.2, 26.1. HRMS (ESI) m/z calcd for C₁₄H₁₆N₂O₄ [M−H]⁻ 275.1037, found 275.1043.

### Example 5

### N-(2-(5-hydroxy-1H-indol-3-yl)ethyl)acetamide-1-¹³C (28)

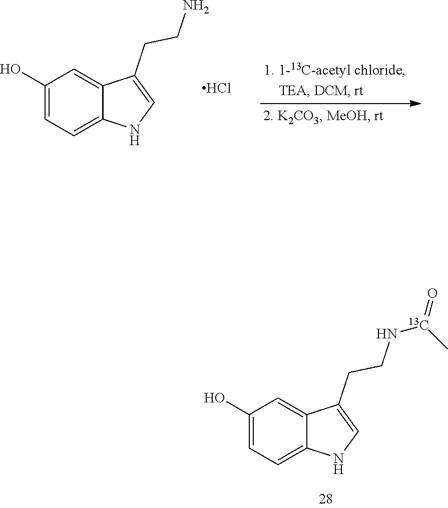 [SMILES: NCCc1c[nH]c2ccc(O)cc12 ; Cl ; C[13C](=O)NCCc1c[nH]c2ccc(O)cc12]

To a suspension of serotonin hydrochloride (132 mg, 0.621 mmol, 1.0 equiv.) in DCM (5 mL) was added TEA (433 μL, 3.10 mmol, 5.0 equiv.). The stirred mixture was cooled to 0° C. before 1-¹³C-acetyl chloride (93 μL, 1.30 mmol, 2.1 equiv.) was added. The mixture was slowly warmed to room temperature and stirred for 24 hours. The reaction mixture was then diluted with DCM, the organics were washed with water, dried with Na₂SO₄, and concentrated in vacuo. Crude intermediates were dissolved in MeOH (10 mL), and K₂CO₃ (85.8 mg, 0.621 mmol, 1.0 equiv.) was added. The reaction was stirred at room temperature for 2 hours and concentrated to 2 mL in vacuo. The residue was diluted with water and extracted with EtOAc twice. The organics were separated, washed with brine, and dried with Na₂SO₄. Flash column chromatography on silica using a gradient of 0-50% MeOH in DCM afforded 28 (98.0 mg, 72%) as light-yellow oil. ¹H NMR (600 MHz, methanol-d₄): δ (ppm) 7.15 (dd, J=0.6, 8.6 Hz, 1H), 6.99 (s, 1H), 6.93 (dd, J=0.6, 2.4 Hz, 1H), 6.66 (dd, J=2.4, 8.6 Hz, 1H), 3.42 (ddd, J=3.7, 7.3, 8.2 Hz, 2H), 2.85 (dt, J=0.6, 7.3 Hz, 2H), 1.91 (d, J=6.1 Hz, 3H). ¹³C NMR (125 MHz, methanol-d₄): δ (ppm) 175.9 (¹²C), 173.4 (¹³C), 151.1, 133.1, 129.5, 124.2, 112.6, 112.4, 103.5, 41.4, 26.2, 22.6 (d, J=50.3 Hz). HRMS (ESI) m/z calcd for C₁₁¹³CH₁₄N₂O₂ [M+H]⁺ 220.1161, found 220.1160.

### Example 6. Steps 1 and 2

### N-(2-(5-(((2R,3R,4S,5R,6R)-3,4,5-tris(benzyloxy)-6-((benzyloxy)methyl)tetrahydro-2H-pyran-2-yl)oxy)-1H-indol-3-yl)ethyl)acetamide (45)

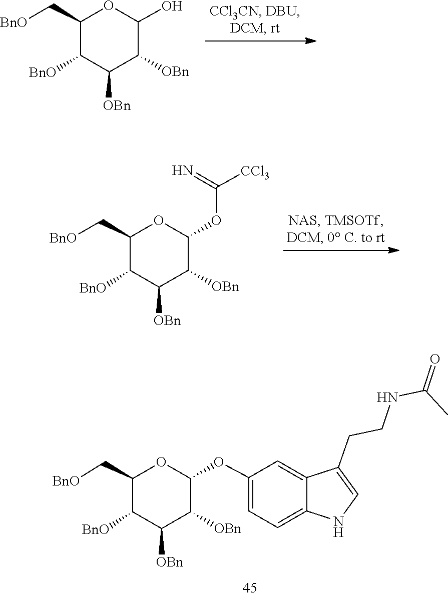

To a solution of 2,3,4,6-tetra-O-benzyl-D-glucopyranose (412 mg, 0.761 mmol, 1.0 equiv.) in DCM (2 mL) was added trichloroacetonitrile (152 μL, 1.52 mmol, 2.0 equiv.) and DBU (21 μL, 0.152 mmol, 0.2 equiv.) under argon. The mixture was stirred at room temperature for 1.5 hours and concentrated in vacuo. Flash column chromatography on silica using a gradient of 25% ethyl acetate in hexanes afforded intermediate 44 (502.4 mg, 97%) as clear oil. A well-stirred solution of 44 (502.4 mg, 0.745 mmol, 2.0 equiv.) and N-acetylserotonin (806 mg, 0.368 mmol, 1.0 equiv.) in DCM (4 mL) and DMF (0.8 mL) was cooled to 0° C., followed by addition of TMSOTf (66 μL, 0.368 mmol, 1.0 equiv.), and the solution was allowed to warm to room temperature within 30 minutes. After stirring at 45° C. for 18 hours, the mixture was concentrated in vacuo. Flash column chromatography on silica using a gradient of 0-15% MeOH in DCM afforded 45 (59.7 mg, 22%) as clear oil. ¹H NMR (500 MHz, chloroform-d): δ (ppm) 7.41-7.26 (m, 20H), 7.17-7.14 (m, 2H), 7.04-7.01 (m, 2H), 5.50 (d, J=3.4 Hz, 1H), 5.44 (m, 1H), 5.08 (d, J=10.8 Hz, 1H), 4.90 (d, J=11.0 Hz, 1H), 4.88 (d, J=10.9 Hz, 1H), 4.81 (d, J=12.0 Hz, 1H), 4.72 (d, J=12.0 Hz, 1H), 4.57 (d, J=11.9 Hz, 1H), 4.50 (d, J=10.8 Hz, 1H), 4.41 (d, J=12.0 Hz, 1H), 4.25 (t, J=9.2 Hz, 1H), 4.03 (m, 1H), 3.78-3.71 (m, 3H), 3.62 (dd, J=1.9, 10.8 Hz, 1H), 3.53 (dt, J=6.2, 6.6 Hz, 2H), 2.87 (t, J=6.6 Hz, 2H), 1.90 (s, 3H). ¹³C NMR (125 MHz, chloroform-d): δ (ppm) 170.1, 151.2, 139.0, 138.4, 138.2, 138.0, 132.7, 128.62, 128.58, 128.54, 128.48, 128.19, 128.13, 128.05, 128.02, 127.87, 127.82, 127.78, 123.1, 114.3, 113.2, 111.9, 105.8, 96.8, 82.2, 80.0, 77.8, 76.0, 75.3, 75.5, 10.8, 68.7, 39.6, 25.4, 23.5.

### Example 6. Step 3

### N-(2-(5-(((2R,3R,4S,5S,6R)-3,4,5-trihydroxy-6-(hydroxymethyl)tetrahydro-2H-pyran-2-yl)oxy)-1H-indol-3-yl)ethyl)acetamide (36)

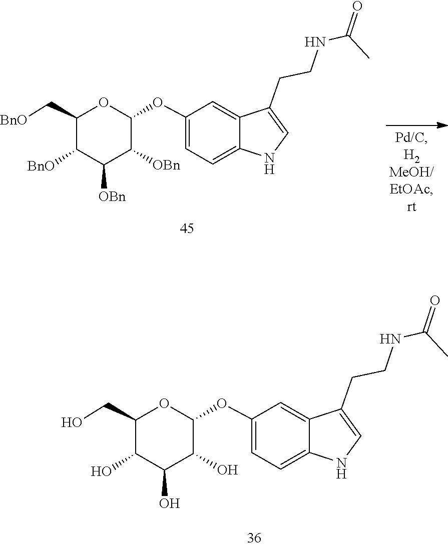 [SMILES: CC(=O)NCCc1c[nH]c2ccc(O[C@H]3O[C@H](COCc4ccccc4)[C@@H](OCc4ccccc4)[C@H](OCc4ccccc4)[C@H]3OCc3ccccc3)cc12 ; CC(=O)NCCc1c[nH]c2ccc(O[C@H]3O[C@H](CO)[C@@H](O)[C@H](O)[C@H]3O)cc12]

To a solution of 45 (59.2 mg, 0.080 mmol, 1.0 equiv.) in a mixture of MeOH and EtOAc (3 mL, v/v=1:1) was added Pd/C (10% w/w, 38 mg). The stirred reaction mixture was purged with argon for 5 minutes, flushed with hydrogen and then subjected to a hydrogen atmosphere for 2 hours at room temperature, and again purged with argon for 5 minutes. The mixture was filtered through Celite and concentrated in vacuo, affording 36 as clear oil (29.8 mg, 98%). HRMS (ESI) m/z calcd for C₁₈H₂₄N₂O₇ [M+Na]⁺ 403.1476, found 403.1486.

### Example 7. Step 1. Synthesis of sngl #101 (37)

### N-(2-(5-hydroxyindolin-3-yl)ethyl)acetamide (46)

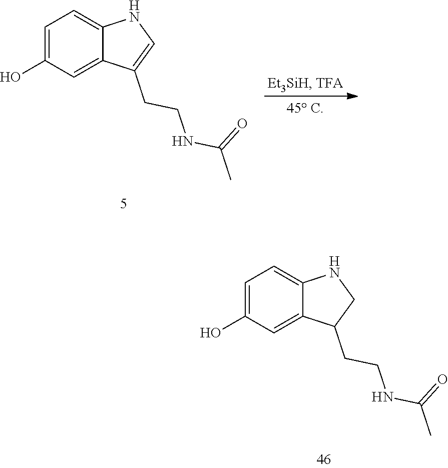 [SMILES: CC(=O)NCCc1c[nH]c2ccc(O)cc12 ; CC(=O)NCCC1CNc2ccc(O)cc21]

To a solution of N-acetylserotonin (210.2 mg, 0.963 mmol, 1.0 equiv.) in TFA (4 mL) was added triethylsilane (185 μL, 1.15 mmol, 1.2 equiv.). The mixture was stirred at 45° C. for 4 hours and concentrated in vacuo. Flash column chromatography on silica using a gradient of 0-40% MeOH in DCM afforded 46 (209.0 mg, 99%). ¹H NMR (500 MHz, methanol-d₄): δ (ppm) 7.17 (d, J=8.6 Hz, 1H), 6.79 (d, J=2.2 Hz, 1H), 6.74 (dd, J=2.2, 8.6 Hz, 1H), 3.95-3.88 (m, 1H), 3.62-3.55 (m, 1H), 3.49-3.42 (m, 2H), 3.29-3.20 (m, 2H), 2.02-1.94 (m, 1H), 1.88 (s, 3H), 1.72-1.63 (m, 1H). ¹³C NMR (125 MHz, methanol-d₄): δ (ppm) 173.5 (br), 160.2, 141.3, 128.5, 120.1, 116.6, 112.7, 52.5, 40.6, 38.0, 34.5, 22.5. HRMS (ESI) m/z calcd for C₁₂H₁₆N₂O [M+H]⁺ 221.1284, found 221.1272.

### Example 7. Step 2

### (2R,3R,4S,5R,6R)-2-(3-(2-acetamidoethyl)-5-acetoxyindolin-1-yl)-6-(acetoxymethyl)tetrahydro-2H-pyran-3,4,5-triyl triacetate (47)

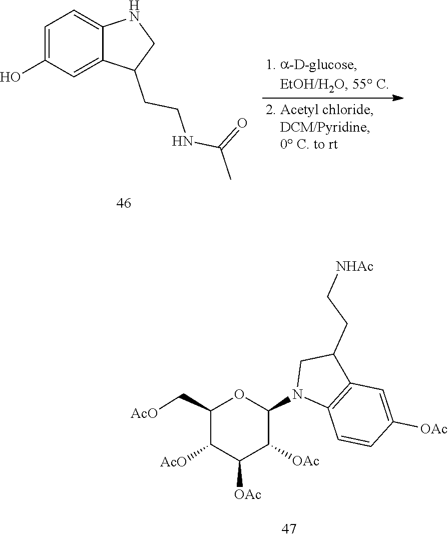 [SMILES: CC(=O)NCCC1CNc2ccc(O)cc21 ; CC(=O)NCCC1CN([C@@H]2O[C@H](COC(C)=O)[C@@H](OC(C)=O)[C@H](OC(C)=O)[C@H]2OC(C)=O)c2ccc(OC(C)=O)cc21]

To a solution of 46 (209 mg, 0.953 mmol, 1.0 equiv.) in TFA (1.5 mL) was added α-D-glucose (867 mg, 4.82 mmol, 5.0 equiv.). The mixture was refluxed for 2 hours and concentrated in vacuo. The crude intermediate was redissolved in pyridine (15 mL) and acetic anhydride (8 mL, 86.7 mmol, 90 equiv.) was added. The resulting mixture was stirred at room temperature for 1 hour and then diluted with water and extracted with DCM:MeOH (v/v=95:5) for three times. The combined organics were washed with sat. aq. NaHCO₃ and brine and dried with Na₂SO₄. Flash column chromatography on silica using a gradient of 0-30% isopropanol in toluene afforded 47 (mixture of diastereomers, 19.5 mg, 3.8%) as yellow oil. ¹H NMR (600 MHz, chloroform-d): δ (ppm) 6.86-6.78 (m, 2H), 6.52 (d, J=8.4 Hz, 0.5H), 6.50 (d, J=8.5 Hz, 0.5H), 5.67 (m, 0.5H), 5.57 (m, 0.5H), 5.33 (dt, J=6.7, 9.4 Hz, 1H), 5.23 (dt, J=8.2, 9.2 Hz, 1H), 5.07 (td, J=3.3, 9.7 Hz, 1H), 4.91 (d, J=10.0 Hz, 1H), 4.25 (ddd, J=5.0, 10.9, 12.4 Hz, 1H), 4.04 (ddd, J=2.4, 12.3, 17.5 Hz, 1H), 3.77-3.71 (m, 2H), 3.34-3.28 (m, 3H), 3.21 (m, 1H), 2.35 (s, 3H), 2.04 (d, J=1.7 Hz, 3H), 2.03 (d, J=1.7 Hz, 3H), 2.01-1.98 (6H), 1.94 (d, J=11.8 Hz, 3H), 1.76-1.62 (m, 2H). HRMS (ESI) m/z calcd for C₂₈H₃₆N₂O₁₂ [M+H]⁺ 593.2341, found 593.2299.

### Example 7. Step 3

### (2R,3R,4S,5R,6R)-2-(3-(2-acetamidoethyl)-5-acetoxy-1H-indol-1-yl)-6-(acetoxymethyl)tetrahydro-2H-pyran-3,4,5-triyl triacetate (48)

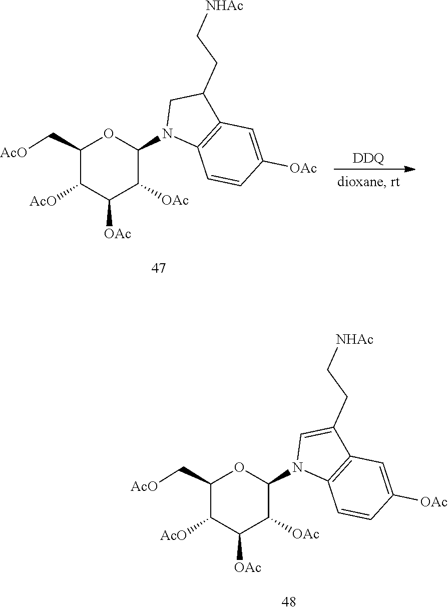

To a solution of 47 (19.5 mg, 0.0324 mmol, 1.0 equiv.) in 1,4-dioxane (1 mL) was added DDQ (8.8 mg, 0.039 mmol, 1.2 equiv.), and the mixture was stirred at room temperature. After 1.5 hours, the reaction mixture was cooled to 0° C. ice bath, diluted with sat. aq. NaHCO₃, and extracted with EtOAc for three times. Combined organics were washed with brine, dried with Na₂SO₄, and then concentrated in vacuo. Flash column chromatography on silica using 100% DCM afforded 48 (15.2 mg, 79%). ¹H NMR (600 MHz, chloroform-d): δ (ppm) 7.31 (d, J=8.9 Hz, 1H), 7.23 (d, J=2.1 Hz, 1H), 7.15 (s, 1H), 6.98 (dd, J=2.1, 8.9 Hz, 1H), 5.91 (m, 1H), 5.53 (d, J=9.0 Hz, 1H), 5.46 (t, J=9.5 Hz, 1H), 5.35 (t, J=9.4 Hz, 1H), 5.25 (t, J=9.8 Hz, 1H), 4.32 (dd, J=5.0, 12.6 Hz, 1H), 4.16 (dd, J=2.1, 12.6 Hz, 1H), 4.11 (q, J=7.2 Hz, 1H), 4.01 (ddd, J=2.2, 5.0, 10.2 Hz, 1H), 3.67 (m, 1H), 3.42 (m, 1H), 2.93 (m, 1H), 2.81 (m, 1H), 2.31 (s, 3H), 2.084 (s, 3H), 2.078 (s, 3H), 2.02 (s, 3H), 1.94 (s, 3H), 1.55 (s, 3H). ¹³C NMR (125 MHz, chloroform-d): δ (ppm) 170.7, 170.6, 170.4, 170.1, 169.6, 169.2, 144.9, 134.6, 128.8, 123.6, 117.1, 115.7, 111.8, 109.8, 83.0, 75.0, 72.8, 71.5, 68.3, 62.0, 51.0, 39.1, 23.3, 21.3, 20.9, 20.73, 20.70, 20.2. HRMS (ESI) m/z calcd for C28H34N₂O₁₂ [M+H]⁺ 591.2184, found 591.2151.

### Example 7. Step 4

### N-(2-(5-hydroxy-1-((2R,3R,4S,5S,6R)-3,4,5-trihydroxy-6-(hydroxymethyl)tetrahydro-2H-pyran-2-yl)-1H-indol-3-yl)ethyl)acetamide (sngl #101, 37)

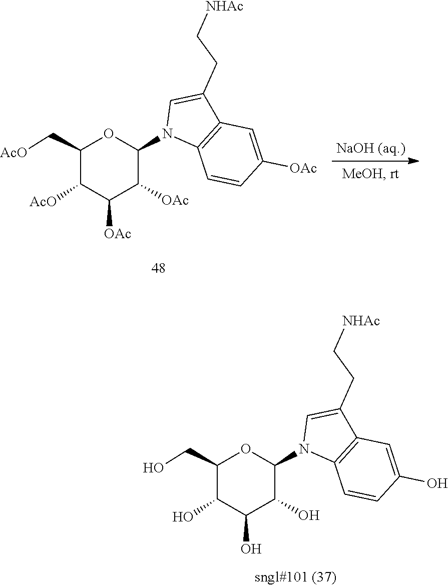 [SMILES: CC(=O)NCCc1cn([C@@H]2O[C@H](COC(C)=O)[C@@H](OC(C)=O)[C@H](OC(C)=O)[C@H]2OC(C)=O)c2ccc(OC(C)=O)cc12 ; CC(=O)NCCc1cn([C@@H]2O[C@H](CO)[C@@H](O)[C@H](O)[C@H]2O)c2ccc(O)cc12]

To a solution of 48 (15.2 mg, 0.0257 mmol, 1.0 equiv.) in MeOH (1.5 mL) was added 8% NaOH (0.3 mL). The mixture was stirred at room temperature for 25 min. and concentrated in vacuo. Flash column chromatography on silica using a gradient of 0-90% MeOH in DCM afforded 37 as clear oil (5.7 mg, 58%). HRMS (ESI) m/z calcd for C₁₈H₂₄N₂O₇ [M+Na]⁺ 403.1476, found 403.1471.

### Example 8. Step 1

### (2R,3R,4S,5S,6R)-2-fluoro-6-(hydroxymethyl)tetrahydro-2H-pyran-3,4,5-triol (38). 38 was prepared as previously described 4,5

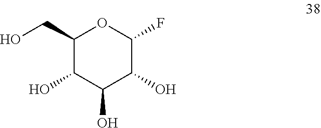 [SMILES: OC[C@H]1O[C@H](F)[C@H](O)[C@@H](O)[C@@H]1O]

### Example 8. Step 2

### N-(2-(5-(((2S,3R,4S,5S,6R)-3,4,5-trihydroxy-6-(hydroxymethyl)tetrahydro-2H-pyran-2-yl)oxy)-1H-indol-3-yl)ethyl)acetamide (sngl #1, 29)

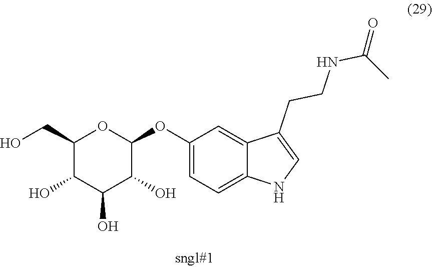 [SMILES: CC(=O)NCCc1c[nH]c2ccc(O[C@@H]3O[C@H](CO)[C@@H](O)[C@H](O)[C@H]3O)cc12]

To a 20 mL glass vial containing 38 (1.52 g, 8.35 mmol, 3 equiv.), N-acetylserotonin (607 mg, 2.78 mmol, 1.0 equiv.) and Ca(OH)₂ (618 mg, 8.35 mmol, 3 equiv.) was added water (3 mL). The reaction mixture was stirred vigorously for 35 minutes. The crude mixture was purified by reversed-phase flash chromatography with a C18 column using a gradient of 0-40% MeOH in H₂O, which afforded sngl #1 (29, 779.0 mg, 74%) as a white solid. HRMS (ESI) m/z calcd for C₁₈H₂₄N₂ NaO₇⁺ [M+Na]⁺ 403.1476, found 403.1485.

### Example 8. Step 3

### ((2R,3S,4S,5R,6S)-6-((3-(2-acetamidoethyl)-1H-indol-5-yl)oxy)-3,4,5-trihydroxytetrahydro-2H-pyran-2-yl)methyl 2-aminobenzoate (sngl #3, 31)

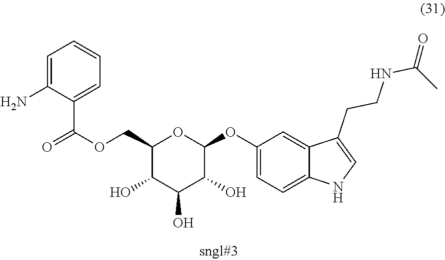 [SMILES: CC(=O)NCCc1c[nH]c2ccc(O[C@@H]3O[C@H](COC(=O)c4ccccc4N)[C@@H](O)[C@H](O)[C@H]3O)cc12]

To a mixture of DCM/DMF (3 mL, v/v=1:2) was added Boc-2-aminobenzoic acid (15.4 mg, 0.065 mmol, 1.2 equiv.) and EDC·HCl (31.2 mg, 0.163 mmol, 3.0 equiv.). The mixture was stirred at room temperature for 30 minutes, and DMAP (26.5 mg, 0.217 mmol, 4.0 equiv.) and sngl #1 (29, 20.6 mg, 0.0542 mmol, 1.0 equiv.) were added. After 5 days, the reaction mixture was concentrated in vacuo. Flash column chromatography on silica using a gradient of 0-30% MeOH in DCM afforded intermediate 49 (4.1 mg, 13%).

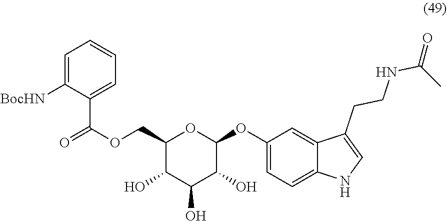 [SMILES: CC(=O)NCCc1c[nH]c2ccc(O[C@@H]3O[C@H](COC(=O)c4ccccc4NC(=O)OC(C)(C)C)[C@@H](O)[C@H](O)[C@H]3O)cc12]

Intermediate 49 was redissolved in DCM (1 mL), followed by slow addition of TFA (0.1 mL). The reaction mixture was stirred at room temperature for 1.5 hours and concentrated in vacuo. Preparative HPLC provided a pure sample of sngl #3 (31, 0.3 mg, 1.1%). HRMS (ESI) m/z calcd for C₂₅H₂₉N₃O₈ [M+H]⁺ 500.2027, found 500.2005.

### Example 8. Step 1

### N-(2-(5-(((6aR,8S,9R,10R,10aS)-9,10-dihydroxy-2,2,4,4-tetraisopropylhexahydropyrano[3,2-f][1,3,5,2,4]trioxadisilocin-8-yl)oxy)-1H-indol-3-yl)ethyl)acetamide (50)

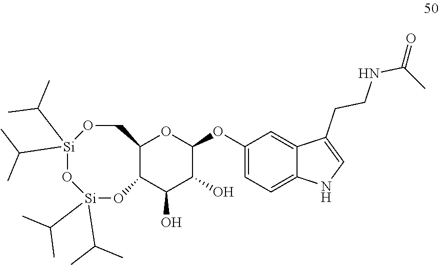 [SMILES: CC(=O)NCCc1c[nH]c2ccc(O[C@@H]3O[C@@H]4CO[Si](C(C)C)(C(C)C)O[Si](C(C)C)(C(C)C)O[C@H]4[C@H](O)[C@H]3O)cc12]

To a solution of sngl #1 (29, 194 mg, 0.511 mmol, 1.0 equiv.) in DMF was added imidazole (152 mg, 1.84 mmol, 4.4 equiv.) was cooled to 0° C. before TIPDSiCl₂ (228 μL, 0.713 mmol, 1.4 equiv.) was added. The reaction mixture was allowed to warm to room temperature over 1.5 hours and stirred for another 30 minutes. The mixture was then diluted with DCM and quenched with water. The organics were washed with sat. aq. NaHCO₃, dried with Na₂SO₄, and concentrated in vacuo. Flash column chromatography on silica using a gradient of 0-10% MeOH in DCM afforded 50 as a white solid (227.6 mg, 72%). ¹H NMR (500 MHz, chloroform-d): δ (ppm) 8.16 (s, 1H), 7.25-7.20 (m, 2H), 7.04-6.95 (m, 2H), 4.89 (d, J=7.3 Hz, 1H), 4.13 (d, J=11.9 Hz, 1H), 4.01 (d, J=12.5 Hz, 1H), 3.93 (t, J=8.9 Hz, 1H), 3.75-3.64 (m. 2H), 3.52 (m, 2H), 3.36 (m, 1H), 3.88 (m, 2H), 1.94 (s, 3H), 1.10-0.99 (m, 28H). HRMS (ESI) m/z calcd for C₃₀H₅₀N₂O₈Si₂, [M+H]⁺ 623.3178, found 623.3157.

### Example 8. Step 2

### (6aR,8S,9R,10R,10aS)-8-((3-(2-acetamidoethyl)-1H-indol-5-yl)oxy)-10-hydroxy-2,2,4,4-tetraisopropylhexahydropyrano[3,2-f][1,3,5,2,4]trioxadisilocin-9-yl benzyl carbonate (51)

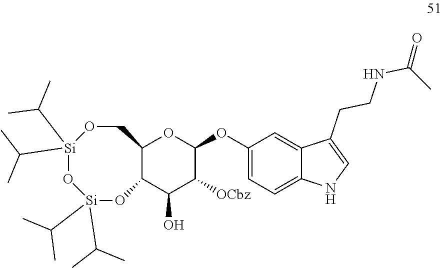

To a solution of 50 (227 mg, 0.365 mmol, 1.0 equiv.) in DCM was added DMAP (147 mg, 1.20 mmol, 3.3 equiv.) and DMF (50 μL). The mixture was cooled to 0° C. before added benzyl chloroformate (233 μL, 1.64 mmol, 4.5 equiv.). The reaction mixture was allowed to warm to room temperature within 30 minutes and stirred for another 1.3 hours. The mixture was diluted with DCM and then quenched with water. The aqueous layer was separated and extracted with DCM for three times. The combined organics were washed with sat. aq. NaHCO₃ and brine, dried with Na₂SO₄, and concentrated in vacuo. Flash column chromatography of the residue on silica using a gradient of 0-20% isopropanol in toluene afforded 51 as a white solid (196.1 mg, 66%). 1H NMR (600 MHz, chloroform-d): δ (ppm) 8.22 (s, 1H), 7.40-7.37 (m, 2H), 7.36-7.31 (m, 3H), 7.19-7.16 (m, 2H), 6.99 (s, 1H), 6.82 (dd, J=2.2, 8.7 Hz, 1H), 5.64 (m, 1H), 5.26 (d, J=12.1 Hz, 1H), 5.21 (d, J=12.1 Hz, 1H), 4.97 (d, J=8.0 Hz, 1H), 4.93 (dd, J=8.7, 9.3 Hz, 1H), 4.12 (dd, J=1.9, 12.7 Hz, 1H), 4.05 (dd, J=1.2, 12.7 Hz, 1H), 3.98 (t, J=1.2, 9.3 Hz, 1H), 3.81 (t, J=1.2, 9.1 Hz, 1H), 3.51-3.47 (m, 2H), 3.34 (dt, J=1.2, 9.4 Hz, 1H), 2.83 (t, J=6.6 Hz, 2H), 1.88 (s, 3H), 1.14-1.01 (m, 28H). ¹³C NMR (125 MHz, chloroform-d): δ (ppm) 170.5, 155.1, 151.7, 135.2, 133.1, 128.74, 128.68, 128.48, 128.35, 127.8, 126.4, 114.5, 113.0, 111.8, 106.6, 101.5, 77.9, 76.7, 75.2, 70.2, 69.7, 60.9, 39.8, 25.2, 23.3, 17.57, 17.47, 17.43, 17.37, 17.33, 17.31, 17.25, 13.7, 13.3, 12.7, 12.6. HRMS (ESI) m/z calcd for C₃₈H₅₆N₂O₁₀Si₂ [M+H]⁺ 757.3546, found 757.3517.

### Example 8. Step 3

### (6aR,8S,9R,10R,10aR)-8-((3-(2-acetamidoethyl)-1H-indol-5-yl)oxy)-10-((bis(benzyloxy)phosphoryl)oxy)-2,2,4,4-tetraisopropylhexahydropyrano[3,2-f][1,3,5,2,4]trioxadisilocin-9-yl benzyl carbonate (52)

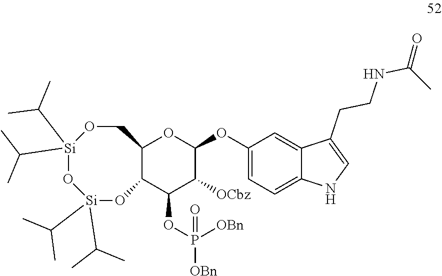

To a solution of 51 (145.8 mg, 0.188 mmol, 1.0 equiv.) in DCM was added dibenzyl N,N-diisopropylphosphoramidite (221 μL, 0.659 mmol, 3.5 equiv.) and 1H-tetrazole (0.45 M in ACN, 1.5 mL, 0.659 mmol, 3.5 equiv.). The reaction mixture was stirred at room temperature for 1 hour. The solution was cooled to −78° C. under argon, and m-CPBA (≤77%, 143.0 mg, 0.638 mmol, 3.4 equiv.) in DCM (1.5 mL) was added slowly to the reaction mixture. The solution was stirred at −78° C. for 0.5 hour, and slowly warmed to room temperature and reacted for another 1 hour, then was diluted with DCM and washed with 10% Na₂SO₄ twice, sat. aq. NaHCO₃, and brine, dried with Na₂SO₄, and concentrated in vacuo. Flash column chromatography on silica using a gradient of 5-100% EtOAc in hexanes afforded 52 as a white solid (141.3 mg, 74%). ¹H NMR (500 MHz, chloroform-d): δ (ppm) 8.31 (s, 1H), 7.36-7.27 (m, 15H), 7.22-7.18 (m, 2H), 7.04 (d, J=2.2 Hz, 1H), 6.78 (dd, J=2.2, 8.7 Hz, 1H), 5.53 (m, 1H), 5.23 (d, J=12.2 Hz, 1H), 5.13 (dd, J=8.0, 9.4 Hz, 1H), 5.08-4.91 (m, 6H), 4.61 (dt, J=8.6, 8.9 Hz, 1H), 4.20-4.14 (m, 2H), 4.09 (d, J=12.6 Hz, 1H), 3.59-3.47 (m, 2H), 3.33 (dt, J=1.7, 9.4 Hz, 1H), 2.87 (t, J=6.6 Hz, 2H), 1.92 (s, 3H), 1.16-0.99 (m, 28H). ¹³C NMR (125 MHz, chloroform-d): 170.2, 154.6, 151.7, 136.14, 136.08, 135.90, 135.85, 135.3, 133.2, 128.60, 128.57, 128.53, 128.49, 128.36, 128.12, 128.06, 127.8, 123.3, 114.6, 113.1, 111.8, 106.8, 101.6, 80.3 (d, J=6.5 Hz), 76.6 (d, J=4.6 Hz), 70.0, 69.6 (t, J=5.9 Hz), 68.7 (d, J=5.2 Hz), 60.9, 39.8, 25.3, 23.5, 17.54, 17.50, 17.46, 17.41, 17.36, 17.34, 17.28, 17.11, 13.35, 13.26, 12.97, 12.95. HRMS (ESI) m/z calcd for C₅₂H₆₉N₂O₁₃PSi₂ [M+H]⁺ 1017.4149, found 1017.4105.

### Example 8. Step 4

### (2S,3R,4S,5R,6R)-2-((3-(2-acetamidoethyl)-1H-indol-5-yl)oxy)-4-((bis(benzyloxy)phosphoryl)oxy)-5-hydroxy-6-(hydroxymethyl)tetrahydro-2H-pyran-3-yl benzyl carbonate (53)

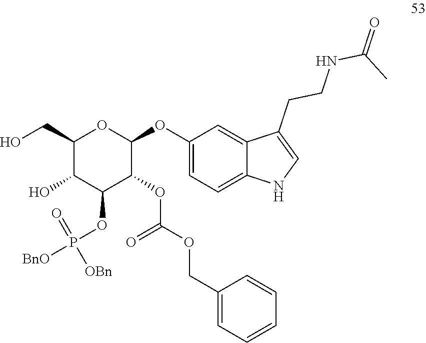 [SMILES: CC(=O)NCCc1c[nH]c2ccc(O[C@@H]3O[C@H](CO)[C@@H](O)[C@H](OP(=O)(OCc4ccccc4)OCc4ccccc4)[C@H]3OC(=O)OCc3ccccc3)cc12]

To a solution of 52 (141.3 mg, 0.139 mmol, 1.0 equiv.) in THF (6 mL) was added acetic acid (24 μL, 0.417 mmol, 3.0 equiv.). The solution was cooled to −10° C. before tetrabutylammonium fluoride solution (1M in THF, 417 μL, 00.417 mmol, 3.0 equiv.) was added. The reaction mixture was stirred for 1.5 hours in cold and concentrated in vacuo. Flash column chromatography on silica using a gradient of 0-15% MeOH in DCM afforded 53 as a white solid (92.3 mg, 86%). ¹H NMR (500 MHz, chloroform-d): δ (ppm) 8.32 (s, 1H), 7.36-7.21 (m, 15H), 7.14 (d, J=8.7 Hz, 1H), 6.96 (d, J=2.1 Hz, 1Hd), 6.71 (dd, J=2.1, 8.7 Hz, 1H), 5.82 (m, 1H), 5.12 (d, J=12.2 Hz, 1H), 5.10-4.94 (m, 7H), 4.49 (dt, J=7.2, 8.9 Hz, 1H), 3.99 (dd, J=2.8, 12.2 Hz, 1H), 3.84-3.74 (m, 2H), 3.55-3.46 (m, 2H), 3.40 (m, 1H), 2.91-2.77 (m, 2H), 1.89 (s, 3H). ¹³C NMR (125 MHz, chloroform-d): δ (ppm) 171.1, 154.5, 151.2, 135.0, 133.1, 128.81, 128.75, 128.72, 128.71, 128.69, 128.66, 123.4, 114.2, 113.1, 111.8, 106.9, 100.6, 81.7 (d, J=5.6 Hz), 76.17, 76.0 (d, J=6.2 Hz), 70.4, 70.25 (d, J=6.0 Hz), 70.17, 70.13 (d, J=6.0 Hz), 62.3, 40.3, 25.4, 23.4. HRMS (ESI) m/z calcd for C₄₀H₄₃N₂O₁₂P [M−H]⁻ 773.2481, found 773.2488.

### Example 8. Step 5

### (2S,3R,4S,5R,6R)-2-((3-(2-acetamidoethyl)-1H-indol-5-yl)oxy)-3,5-dihydroxy-6-(hydroxymethyl)tetrahydro-2H-pyran-4-yl dihydrogen phosphate (30)

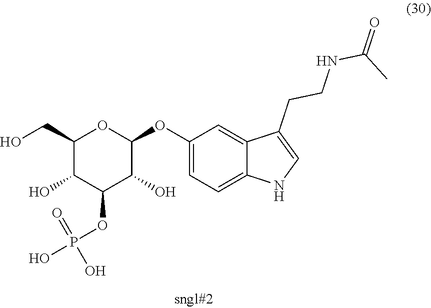 [SMILES: CC(=O)NCCc1c[nH]c2ccc(O[C@@H]3O[C@H](CO)[C@@H](O)[C@H](OP(=O)(O)O)[C@H]3O)cc12]

To a solution of 53 (26.9 mg, 0.0347 mmol, 1.0 equiv.) in a mixture of MeOH and EtOAc (2 mL, v/v=1:1) was added Pd/C (10% w/w, 18 mg). The reaction mixture was purged with argon for 5 minutes, flushed with hydrogen, and then subjected to hydrogen atmosphere for 1.5 hours at room temperature, and subsequently again purged with argon for 5 minutes. The mixture was filtered through Celite and concentrated in vacuo. The crude mixture was purified by reversed-phase flash chromatography with a C18 column using a gradient of 0-10% ACN in H₂O with 0.1% formic acid, which afforded sngl #2 as a clear oil (30, 9.3 mg, 58%). HRMS (ESI) m/z calcd for C₁₈H₂₅N₂O₁₀P [M−H]⁻ 459.1174, found 459.1185.

### Example 9. Step 1

((2R,3R,4S,5R,6S)-6-((3-(2-acetamidoethyl)-1H-indol-5-yl)oxy)-5-(((benzyloxy)carbonyl)oxy)-4-((bis(benzyloxy)phosphoryl)oxy)-3-hydroxytetrahydro-2H-pyran-2-yl)methyl 2-((tert-butoxycarbonyl)amino)benzoate (54)

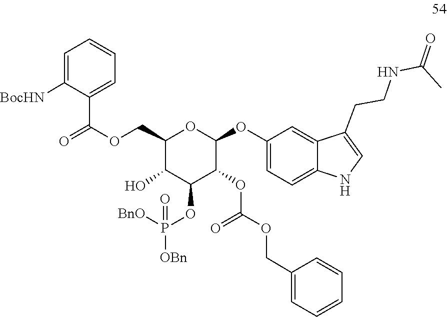 [SMILES: CC(=O)NCCc1c[nH]c2ccc(O[C@@H]3O[C@H](COC(=O)c4ccccc4NC(=O)OC(C)(C)C)[C@@H](O)[C@H](OP(=O)(OCc4ccccc4)OCc4ccccc4)[C@H]3OC(=O)OCc3ccccc3)cc12]

To a mixture of dry DCM/DMF (2 mL, v/v=100:1) was added Boc-2-aminobenzoic acid (70.7 mg, 0.298 mmol, 2.5 equiv.) and EDC·HCl (68.4 mg, 0.444 mmol, 3.0 equiv.). The mixture was stirred at room temperature for 25 minutes, and DMAP (58.2 mg, 0.476 mmol, 4.0 equiv.) and 53 (92.3 mg, 0.119 mmol, 1.0 equiv.) were added. After 25 hours, the reaction mixture was concentrated in vacuo. Flash column chromatography on silica using a gradient of 0-12% MeOH in DCM afforded 54 as a white solid (72.0 mg, 61%). ¹H NMR (600 MHz, chloroform-d): δ (ppm) 8.42 (d, J=8.4 Hz, 1H), 7.97 (dd, J=1.1, 8.0 Hz, 1H), 7.49 (dd, J=1.1, 7.8 Hz, 1H), 7.33-7.15 (m, 16H), 7.09 (d, J=8.7 Hz, 1H), 7.00 (d, J=1.1 Hz, 1H), 6.91 (t, J=7.8 Hz, 1H), 6.82 (dd, J=2.0, 8.7 Hz, 1H), 5.61 (m, 1H), 5.15-4.95 (m, 8H), 4.71 (dd, J=2.0, 12.0 Hz, 1H), 4.56 (dd, J=6.1, 12.0 Hz, 1H), 4.49 (m, 1H), 3.84 (t, J=9.4 Hz, 1H), 3.77 (m, 1H), 3.49-3.44 (m, 2H), 2.75 (t, J=6.9 Hz, 2H), 1.90 (s, 3H), 1.50 (s, 9H). HRMS (ESI) m/z calcd for C₅₂H₅₆N₃O₁₅P [M+H]⁺ 994.3522, found 994.3489.

### Example 9. Step 2

### ((2R,3R,4S,5R,6S)-6-((3-(2-acetamidoethyl)-1H-indol-5-yl)oxy)-5-(((benzyloxy)carbonyl)oxy)-4-((bis(benzyloxy)phosphoryl)oxy)-3-hydroxytetrahydro-2H-pyran-2-yl)methyl 2-aminobenzoate (55)

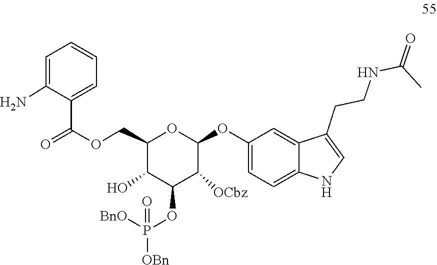

To a solution of 54 (72.0 mg, 72.5 μmol, 1.0 equiv.) in DCM (2 mL) was added TFA (200 μL). The yellow mixture was stirred at room temperature for 1 hour and turned purple. The reaction mixture was then concentrated in vacuo. Flash column chromatography on silica using a gradient of 0-10% MeOH in DCM afforded 55 (54.9 mg, 85%). 1H NMR (600 MHz, acetone-d₆): δ (ppm) 7.89 (dd, J=1.5, 8.1 Hz, 1H), 7.40-7.24 (m, 17H), 7.22 (d, J=8.5 Hz, 1H), 7.15 (s, 1H), 6.84 (dd, J=2.3, 8.7 Hz, 1H), 6.80 (dd, J=0.6, 8.3 Hz, 1H), 6.56 (ddd, J=1.1, 7.1, 8.3 Hz, 1H), 5.32 (d, J=8.1 Hz, 1H), 5.24 (d, J=12.2 Hz, 1H), 5.17-5.03 (m, 6H), 4.84 (m, 1H), 4.75 (dd, J=1.2, 12.2 Hz, 1H), 4.53 (dd, J=5.6, 11.8 Hz, 1H), 4.10 (m, 1H), 4.03 (t, J=9.1 Hz, 1H), 3.50-3.39 (m, 2H), 2.83 (t, J=7.2 Hz, 2H), 1.87 (s, 3H). ¹³C NMR (125 MHz, acetone-d₆): δ (ppm) 168.4, 155.4, 152.4, 152.0, 137.1, 136.5, 132.0, 129.34, 129.32, 129.26, 129.18, 129.13, 129.07, 129.04, 128.84, 128.81, 128.68, 124.44, 117.3, 116.1, 113.8, 113.5, 112.5, 110.4, 106.8, 101.1, 81.6 (d, J=5.8 Hz), 77.1 (d, J=4.6 Hz), 74.6, 70.54, 70.46, 70.3 (d, J=5.5 Hz), 70.2 (d, J=5.5 Hz), 63.7, 40.4, 26.3, 23.0. HRMS (ESI) m/z calcd for C₄₇H₅₆N₃₀₁₅P [M+H]⁺ 894.2998, found 894.2957.

### Example 9. Step 3

### ((2R,3R,4S,5R,6S)-6-((3-(2-acetamidoethyl)-1H-indol-5-yl)oxy)-3,5-dihydroxy-4-(phosphonooxy)tetrahydro-2H-pyran-2-yl)methyl 2-aminobenzoate (sngl #4, 32)

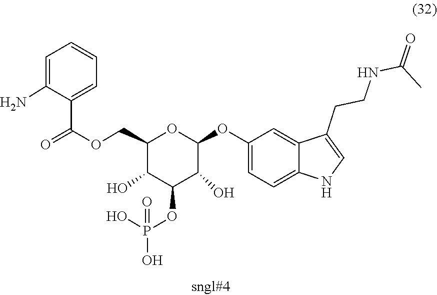 [SMILES: CC(=O)NCCc1c[nH]c2ccc(O[C@@H]3O[C@H](COC(=O)c4ccccc4N)[C@@H](O)[C@H](OP(=O)(O)O)[C@H]3O)cc12]

To a solution of 55 (54.9 mg, 61.4 μmol, 1.0 equiv.) in a mixture of MeOH and EtOAc (2.5 mL, v/v=2:3) was added Pd/C (10% w/w) (32 mg). The reaction mixture was purged with argon for 5 minutes, flushed with hydrogen, and then subjected to hydrogen atmosphere for 3 hours at room temperature, and again purged with argon for 5 minutes. The mixture was filtered through Celite and concentrated in vacuo, affording sngl #4 (32, 33.4 mg, 94%). HRMS (ESI) m/z calcd for C33H36N₃₀₁₃P, [M−H]⁻ 578.1545, found 578.1554.

### Synthesis of Glucosyladenine Derivatives

### Example 10. Step 1. N⁹—(β-glucopyranosyl)-N⁶-methyladenine (BC-2, maglu #3)

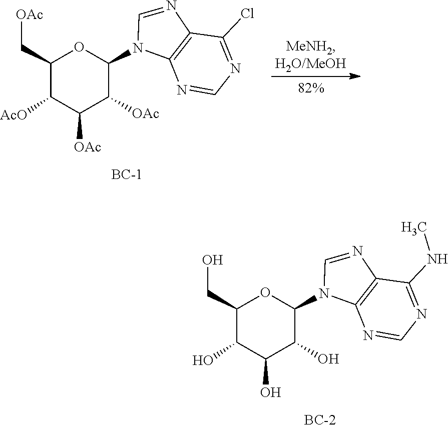

To BC-1 (503 mg, 0.62 mmol, 1.00 equiv.) in a high-pressure flask was added 15 mL of MeNH₂ (40% in H₂O) and 2 mL MeOH. The flask was sealed and heated to 100° C., at which the solution was stirred for 2 hr. The resulting solution was allowed to cool to room temp, at which a precipitate slowly formed, filtered, and washed with cold methanol/water, affording BC-2 (maglu #3, 266 mg, 82%) as a white solid. ¹H NMR (600 MHz, DMSO-d₆): δ 8.30 (s, 1H), 8.23 (br s, 1H), 7.70 (br s, 1H), 5.40 (d, J=9.4 Hz, 1H), 5.31 (d, J=5.8 Hz, 1H), 5.28 (d, J=4.6 Hz, 1H), 5.14 (d, J=5.4 Hz, 1H), 4.59 (t, J=5.9 Hz, 1H), 3.99 (td, J=9.1, 5.8 Hz, 1H), 3.70 (ddd, J=11.7, 5.7, 1.7 Hz, 1H), 3.43 (dt, J=11.9, 6.1 Hz, 1H), 3.41-3.34 (m, 2H), 3.24 (td, J=9.2, 5.6 Hz, 1H), 2.95 (br s, 3H). ¹³C NMR (126 MHz, DMSO-d₆): δ 155.0, 152.6, 139.4, 82.8, 80.0, 77.3, 71.3, 69.8, 60.9, 29.7. HRMS (ESI) m/z: [M+H]⁺ calcd for C₁₂H₁₈N₅O₅ 312.1302; found 312.1290.

### Example 11. Step 1. N⁹—(β-glucopyranosyl)adenine (BC-3)

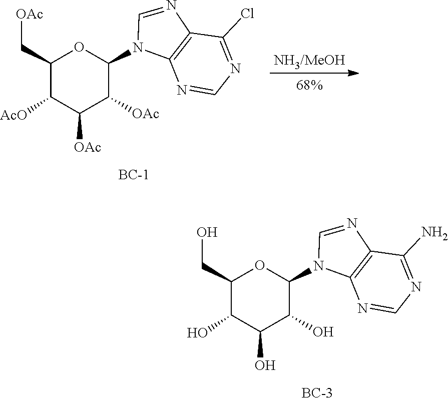

To BC-1 (1.00 g, 2.06 mmol, 1.00 equiv.) in a high-pressure flask was added 5 mL of MeOH and methanolic ammonia (7N, 29 mL, 206 mmol, 100 equiv.). The flask was sealed and heated to 100° C., at which the resulting yellow solution was stirred for 8 hr. The solution was transferred to a round-bottom flask and concentrated to dryness in vacuo. The reaction crude was then re-dissolved in MeOH upon heating, silica gel (11 g) was added, and the mixture was concentrated to dryness in vacuo (for dry-loading). Flash column chromatography on silica using a gradient of 30-60% MeOH in DCM was performed, affording BC-3 (420 mg, 68%) as an off-white power. ¹H NMR (600 MHz, DMSO-d₆): δ 8.31 (s, 1H), 8.14 (s, 1H), 7.23 (s, 2H), 5.39 (d, J=9.4 Hz, 1H), 5.30 (d, J=5.8 Hz, 1H), 5.25 (d, J=4.7 Hz, 1H), 5.12 (d, J=5.6 Hz, 1H), 4.57 (t, J=5.9 Hz, 1H), 3.99 (td, J=9.1, 5.8 Hz, 1H), 3.70 (ddd, J=11.7, 5.7, 1.7 Hz, 1H), 3.43 (dt, J=11.9, 6.1 Hz, 1H), 3.41-3.34 (m, 2H), 3.24 (td, J=9.2, 5.6 Hz, 1H). ¹³C NMR (126 MHz, DMSO-d₆): δ 156.0, 152.6, 149.8, 139.7, 118.7, 82.8, 80.0, 77.3, 71.2, 69.8, 60.9. HRMS (ESI) m/z: [M+H]⁺ calcd for C₁₁H₁₆N₅O₅ 298.1146; found 298.1136.

### Example 12. Step 1. N⁹—(β-glucopyranosyl)-N¹-methyladenine (BC-4, maglu #1)

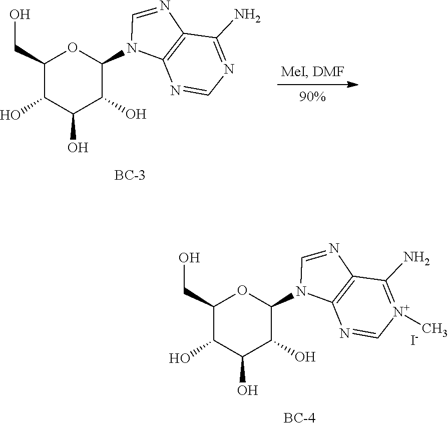

A solution of BC-3 (15 mg, 0.050 mmol, 1.00 equiv.) and Mel (12 μL, 0.193 mmol, 3.85 equiv.) in DMF (0.5 mL) was stirred for 48 hr at 40° C. The resulting yellow solution was concentrated to dryness in vacuo. Flash column chromatography on C18 using 100% H₂O (w/ 0.1% acetic acid) afforded maglu #1 (BC-4, 20 mg, 90%) as a white solid. maglu #1 was compared to the corresponding peak in C. elegans wildtype (N₂) endo-metabolome samples by HILIC-HRMS (Method C) and MS² (see Figure Sla and S5 for co-elution and MS² data, respectively). 1H NMR (500 MHz, methanol-d₄): 8.56 (s, 1H), 8.55 (s, 1H), 5.63 (d, J=9.3 Hz, 1H), 4.02 (t, J=9.0 Hz, 1H), 3.91 (s, 3H), 3.88 (d, J=12.1 Hz, 1H), 3.73 (dd, J=12.1, 5.3 Hz, 1H), 3.62-3.56 (m, 2H), 3.53 (t, J=9.1 Hz). ¹³C NMR (126 MHz, methanol-d₄): δ 152.7, 149.1, 148.7, 144.3, 120.3, 85.2, 81.3, 78.5, 73.7, 70.9, 62.3, 38.3. HRMS (ESI) m/z: [M+H]⁺ calcd for C₁₂H₁₈N₅O₅ 312.1302; found 312.1294.

### Example 13. Step 1. 6′-O, 4′-O-TIPDSi-N⁹—(β-glucopyranosyl)adenine (BC-5)

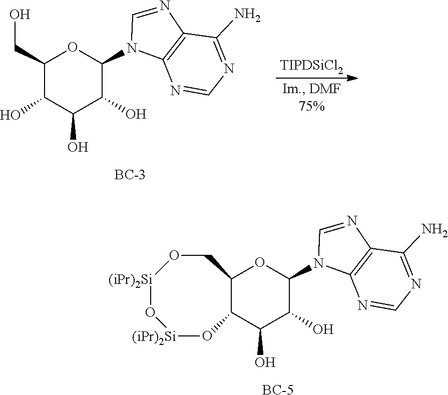

To a solution of BC-3 (350 mg, 1.18 mmol, 1.00 equiv.) in DMF (7 mL) at 0° C. was added TIPDSiCl₂ (560 μL, 1.75 mmol, 1.48 equiv.) and imidazole (362 mg, 5.32 mmol, 4.51 equiv.). The reaction mixture was stirred for 15 min at 0° C. and then diluted with DCM, followed by addition of H₂O. Organics were extracted 2× with DCM, combined, and then basified using sat. aq. NaHCO₃. The organic layer was collected and remaining organics were extracted 3× with a 2:1 mixture of DCM:EtOAc. Combined organics were dried using MgSO₄, filtered, and concentrated in vacuo. The reaction crude was then dissolved in a DCM/MeOH mixture, silica gel (2 g) was added, and the mixture was concentrated to dryness (for dry-loading). Flash column chromatography on silica using a gradient of 2.5-30% MeOH in DCM was performed, affording BC-5 (475 mg, 75%) as a white solid. ¹H NMR (500 MHz, methanol-d₄): δ 8.29 (s, 1H), 8.21 (s, 1H), 5.58 (d, J=9.4 Hz, 1H), 4.18 (dd, J=12.7, 2.2 Hz, 1H), 4.04 (t, J=9.2 Hz, 1H), 3.95 (t, J=9.1 Hz, 1H), 3.91 (dd, J=12.7, 0.8 Hz, 1H), 3.65 (t, J=9.0 Hz, 1H), 3.55 (dt, J=9.4, 1.8 Hz, 1H), 1.27-0.98 (m, 28H). ¹³C NMR (126 MHz, methanol-d₄): δ 157.4, 154.0, 151.2, 140.8, 120.0, 84.8, 80.8, 78.3, 73.7, 70.4, 62.1, 18.0, 17.8, 17.7, 17.6, 14.9, 14.5, 14.0, 13.8.

### Example 13. Step 2. Compounds BC-6 and BC-7

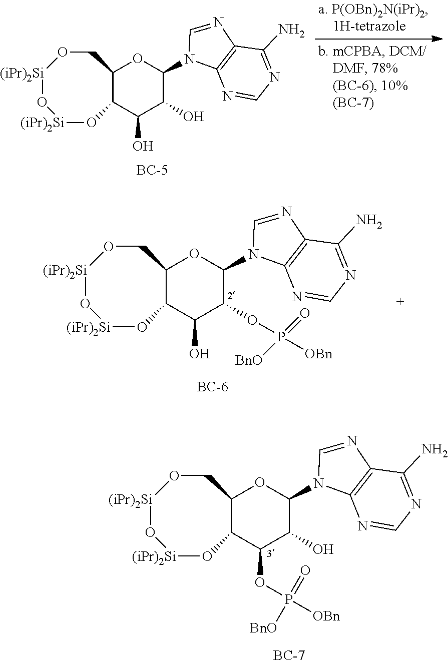

To a solution of BC-5 (260 mg, 0.48 mmol, 1.00 equiv.) in 1:1 DCM:DMF (16 mL) at 40° C. was added dibenzyl N,N-diisopropylphosphoramidite (0.58 mL, 1.73 mmol, 3.60 equiv.), and 1H-tetrazole (0.45 M in ACN, 3.20 mL, 1.44 mmol, 3.00 equiv.). The reaction mixture was stirred at 40° C. for 1 hr and then cooled to −78° C. after which mCPBA (77% max, 300 mg, 1.34 mmol, 2.79 equiv.) was added. The resulting mixture was stirred at −78° C. for 10 min. and was then quenched with the addition of sat. aq. NaHCO₃ (3 mL) after which H₂O (10 mL) and DCM (50 mL) were added. The organic layer was washed 1× with sat. aq. NaHCO₃ (10 mL total) and collected and the aqueous layer was extracted 2× with DCM (20 mL each). Combined organics were dried with MgSO₄, filtered, and concentrated in vacuo. Flash column chromatography on silica using a gradient of 2.5-25% MeOH in DCM was performed, affording separable BC-6 (301 mg, 78%) and BC-7 (40 mg, 10%) as white solids. 2′-O isomer (BC-6): ¹H NMR (600 MHz, methanol-d₄): δ 8.30 (s, 1H), 8.14 (s, 1H), 7.33-7.22 (m, 6H), 7.24-7.17 (m, 2H), 7.00 -6.95 (m, 2H), 5.87 (d, J=9.3 Hz, 1H), 4.90 (dd, J=11.8, 7.5 Hz, 1H), 4.95-4.88 (m, 1H) 4.81 (dd, J=11.7, 8.5 Hz, 1H), 4.53-4.42 (m, 2H), 4.18 (dd, J=12.9, 2.1 Hz, 1H), 4.03 (t, J=9.2 Hz, 1H), 3.94 (dd, J=13.0, 1.2 Hz, 1H), 3.92 (t, J=9.0 Hz, 1H), 3.62 (dt, J=9.4, 1.8 Hz, 1H), 1.31-0.95 (m, 28H). 2′-O isomer (BC-6): ¹³C NMR (126 MHz, methanol-d₄): δ 157.4, 154.1, 151.1, 140.9, 137.0 (d, J=7.3 Hz), 136.5 (d, J=7.3 Hz), 129.6, 129.5, 129.0, 128.6, 120.0, 82.9, 81.0, 80.1, 76.7 (d, J=2.7 Hz), 70.9 (d, J=6.0 Hz), 70.6, 70.4 (d, J=5.9 Hz), 61.9, 18.0, 17.8, 17.7, 17.6, 14.8, 14.5, 14.1, 13.8. 3′-O isomer (BC-7): ¹H NMR (600 MHz, methanol-d₄): δ 8.31 (s, 1H), 8.22 (s, 1H), 7.38-7.30 (m, 10H), 5.65 (d, J=9.2 Hz, 1H), 5.14-5.07 (m, 2H), 5.07-5.00 (m, 2H), 4.55 (q, J=8.5 Hz, 1H), 4.43 (t, J=9.1 Hz, 1H), 4.18 (t, J=9.1 Hz, 1H), 4.15 (dd, J=12.8, 2.0 Hz, 1H), 3.94 (dd, J=12.8, 1.8 Hz, 1H), 3.59 (dt, J=9.4, 1.9 Hz, 1H), 1.14-0.86 (m, 28H). 3′-O isomer (BC-7): ¹³C NMR (126 MHz, methanol-d₄): δ 157.4, 154.0, 151.1, 141.1, 137.4 (d, J=7.0 Hz), 137.1 (d, J=7.2 Hz), 129.7, 129.6, 129.3, 120.1, 86.00, 85.4 (d, J=6.6 Hz), 85.0, 80.3, 72.2, 71.1 (d, J=5.6 Hz), 70.8 (d, J=5.3 Hz), 69.8 (d, J=5.4 Hz), 62.0, 18.0, 17.9, 17.8, 17.7, 17.5, 14.4, 14.3, 14.1.

Note: Some variability between experiments regarding amount of 1H-tetrazole and phosphoramidite needed. It is important to monitor conversion of sugar starting material to prevent bis-phosphorylation. Developed TLC plate (12:1 DCM:MeOH) was visualized using p-anisaldehyde stain, where 2′-O phosphate BC-6 stained brown and 3′-O phosphate BC-7 blue.

### Example 13. Step 3. Compounds BC-8 and BC-9

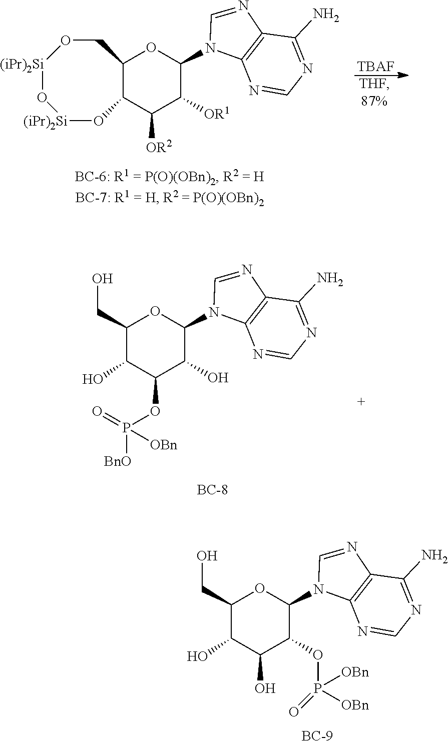

To a 10:1 solution of BC-6 and BC-7 (154 mg, 0.192 mmol, 1.00 equiv.), respectively, in THF (4 mL) at 0° C. was added TBAF (1M in THF, 480 μL, 0.48 mmol, 2.50 equiv.). After 15 min., AcOH (60 uL) was added, and the resulting solution was concentrated in vacuo. Flash column chromatography on silica using a gradient of 10-40% MeOH in DCM was performed, affording BC-8 (73 mg, 0.131 mmol, 68%) and BC-9 (20 mg, 0.036 mmol, 19%) which were able to be mostly separated after subsequent purification. 2′-O isomer (BC-9): ¹H NMR (500 MHz, methanol-d₄): δ 8.38 (s, 1H), 8.15 (s, 1H), 7.34-7.15 (m, 8H), 6.98-6.92 (m, 8H), 5.86 (d, J=9.3 Hz, 1H), 4.97-4.88 (m, 2H), 4.83 (dd, J=12.0, 8.5 Hz, 1H), 4.48 (dd, J=11.8, 6.8 Hz, 1H), 4.43 (dd, J=11.8, 8.5 Hz, 1H), 3.90 (dd, J=12.3, 1.5, 1H), 3.83 (t, J=8.8 Hz, 1H), 3.76 (dd, J=12.3, 5.0 Hz, 1H), 3.68-3.62 (m, 2H). 2′-O isomer (BC-9): ¹³C NMR (126 MHz, methanol-d₄): δ 157.4, 154.1, 150.9, 141.4, 137.0 (d, J=7.2 Hz), 136.6 (d, J=7.4 Hz) 129.5, 128.9, 128.5, 120.0, 82.8, 81.4, 79.7, 77.3 (d, J=2.6 Hz), 71.1, 70.9 (d, J=5.8 Hz), 70.3 (d, J=6.0 Hz), 62.2. 3′-O isomer (BC-8): ¹H NMR (500 MHz, methanol-d₄): δ 8.32 (s, 1H), 8.22 (s, 1H), 7.40-7.24 (m, 10H), 5.65 (d, J=9.3 Hz, 1H), 5.16 (d, J=7.5 Hz, 2H), 5.07-5.00 (m, 2H), 4.54 (q, J=8.8 Hz, 1H), 4.40 (t, J=9.2 Hz, 1H), 3.91 (dd, J=12.2, 2.1, 1H), 3.87 (t, J=9.0 Hz, 1H), 3.80 (dd, J=12.2, 5.1 Hz, 1H), 3.67 (ddd, J=9.8, 5.0, 2.1 Hz, 1H). 3′-O isomer (BC-8): ¹³C NMR (126 MHz, methanol-d₄): δ 157.4, 153.9, 150.8, 141.6, 137.5 (d, J=4.2 Hz), 137.4 (d, J=4.2 Hz), 129.6, 129.5, 129.0, 120.2, 85.9, 85.2, 80.7, 71.9 (d, J=3.2 Hz), 70.8 (d, J=5.8 Hz), 69.7 (d, J=3.2 Hz), 62.1. Note: Developed TLC plate (5:1 DCM:MeOH) was visualized using p-anisaldehyde stain, where 2′-O phosphate BC-9 stained brown and 3′-O phosphate BC-8 blue.

### Example 13. Step 4. N⁹—(β-glucopyranosyl)-(3′-O-phospho)-N¹-methyladenine (maglu #2, BC-10)

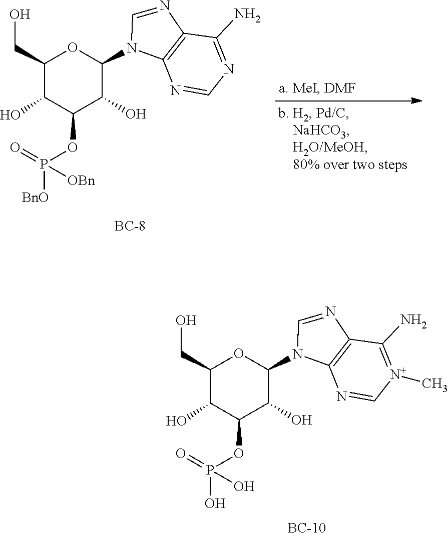 [SMILES: Nc1ncnc2c1ncn2[C@@H]1O[C@H](CO)[C@@H](O)[C@H](OP(=O)(OCc2ccccc2)OCc2ccccc2)[C@H]1O ; "BC-8" ; C[n+]1cnc2c(ncn2[C@@H]2O[C@H](CO)[C@@H](O)[C@H](OP(=O)(O)O)[C@H]2O)c1N ; "BC-10"]

A solution of BC-8 (12 mg, 0.021 mmol) and Mel (5.2 μL, 0.083 mmol, 3.97 equiv.) in DMF (1 mL) was stirred for 24 hr at 40° C. The resulting yellow solution was then dissolved in 1.5 mL MeOH/1.0 mL H₂O. NaHCO₃ (7.5 mg, 0.089 mmol, 4.25 equiv.) and Pd/C (12 mg) were added, the suspension was sparged with Ar for 5 min. then switched to H₂, through which the suspension was rapidly stirred under for 2 hr. After sparging with Ar, AcOH (20 μL) was added and the reaction mixture was filtered through celite. The collected filtrate was concentrated to dryness in vacuo. Flash column chromatography on C18 using 100% H₂O (w/ 0.1% formic acid) afforded maglu #2 (BC-10, 6.8 mg, 80% over two steps) as a white solid. maglu #2 was compared to the corresponding peak in C. elegans wildtype (N₂) endo-metabolome samples by HILIC-HRMS (Method D) and MS² (see Figure S2b and S7 for co-elution and MS² data, respectively). HRMS (ESI) m/z: [M+H]⁺ calcd for C12H19N₅OsP+392.0966; found 392.0953. Note: Addition of NaHCO₃ was required as non-basified solutions led to only mono-debenzylation.^{reference} AcOH was added to prevent any partial Dimroth rearrangement of samples during the concentration step as well during evaluation of sample purity w/crude NMR.

### Example 14. Step 1. Compound BC-11

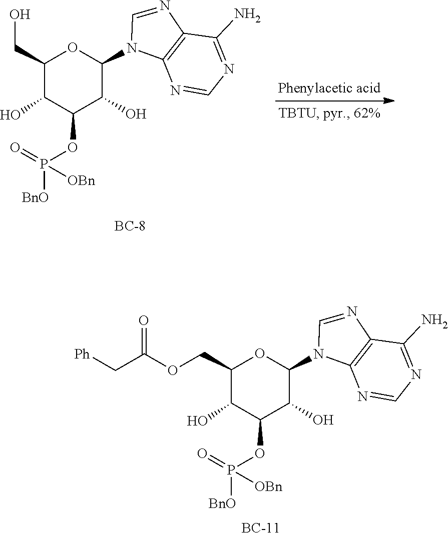

Phenylacetic acid (19 mg, 0.141 mmol, 2.82 equiv.) and TBTU (45 mg, 0.141 mmol, 2.82 equiv.) were added to a solution of BC-8 (28 mg, 0.050 mmol, 1.00 equiv.) in 0.7 mL dry pyridine. The resulting mixture was stirred for 3 hr at room temp. and then concentrated to dryness in vacuo. Flash column chromatography on silica using a gradient of 2.5-30% MeOH in DCM was performed, affording BC-11 (21 mg, 62%) as a colorless oil, with some fractions containing co-eluting HOBt. ¹H NMR (600 MHz, methanol-d₄): δ 8.21 (s, 1H), 8.15 (s, 1H), 7.38-7.26 (m, 1OH), 7.22-7.12 (m, 5H), 5.63 (d, J=9.3 Hz, 1H), 5.17-5.07 (m, 4H), 4.58-4.49 (m, 2H), 4.35 (t, J=9.2 Hz, 1H), 4.32 (dd, J=12.4, 5.9 Hz), 3.88 (ddd, J=10.0, 5.8, 2.2 Hz, 1H), 3.79 (t, J=9.5 Hz, 1H), 3.63 (dd, J=15.0 Hz, 1H), 3.59 (d, J=15.0 Hz, 1H). ¹³C NMR (126 MHz, methanol-d₄): δ 173.2, 157.5, 154.0, 151.0, 150.1, 141.3, 137.5 (d, J=2.8 Hz, 1H), 137.4 (d, J=2.8 Hz, 1H), 135.6, 130.4, 129.6, 129.5, 129.4, 129.0, 128.0, 125.6, 120.3, 85.6 (d, J=6.7 Hz, 1H), 84.8, 77.9, 71.9 (d, J=3.8 Hz, 1H), 70.8 (d, J=5.8 Hz, 1H), 70.1 (d, J=3.8 Hz, 1H), 64.4, 41.8.

### Example 14. Step 2. maglu #11 (BC-12)

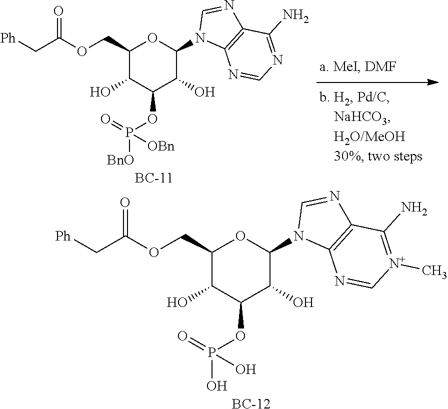

A solution of BC-11 (6.0 mg, 8.88 μmol, 1.00 equiv.) and Mel (2.2 μL, 35.6 μmol, 4.01 equiv.) in dry DMF (0.3 mL) was stirred for 24 hr at 40° C. The resulting yellow solution (a 2:1 mixture of mono and bis-benzylated products, respectively) was concentrated in vacuo and then dissolved in 0.5 mL MeOH and 75 μL H₂O. To the solution was added NaHCO₃ (2.5 mg, 30.0 μmol, 3.38 equiv.) in 22.5 μL H₂O and Pd/C (17 mg, 10% w/w). The suspension was sparged with Ar for 5 min. then switched to H₂, through which the suspension was rapidly stirred under for 1.5 hr. After sparging with Ar for 5 min., AcOH (30 μL) was added and the reaction mixture was then filtered through celite using MeOH/H₂O. The collected filtrate was concentrated to dryness in vacuo. Purification by preparative HPLC (see Methods) afforded maglu #11 (BC-12, 1.4 mg, 30% over two steps) as a white solid. maglu #3 was found to be identical to the corresponding peak on C18 in C. elegans wildtype (N₂),fem-3 (gf), him-5 endo-metabolome samples by HPLC-HRMS (Method A) and MS² (see FIG. S3b and S9 for co-elution and MS² data, respectively). Bis-benzylated and mono-benzylated species—HRMS (ESI) m/z: [M+H]⁺ calcd for C₃₄H₃₇N₅O₉P⁺ 690.2323 found 690.2310 and [M+H]⁺ calcd for C₂₇H₃₁N₅O₉P⁺ 600.1854 found 600.1840 for bis-benzylated and mono-benzylated species, respectively. maglu #3 (BC-12)—HRMS (ESI) m/z: [M+H]⁺ calcd for C₂₀H₂₅N₅O₉P⁺ 510.1384; found 510.1398. A bis-methylated species (c.a. 25%) was also observed. [M+H]⁺ calcd for C₂₁H₂₇N₅O₉P⁺ 524.1541; found 524.1556. This impurity was removed via preparative HPLC (see Methods).

### Synthesis of Glucosyl Guanine Derivatives

### Example 15. Step 1. Compound BC-14

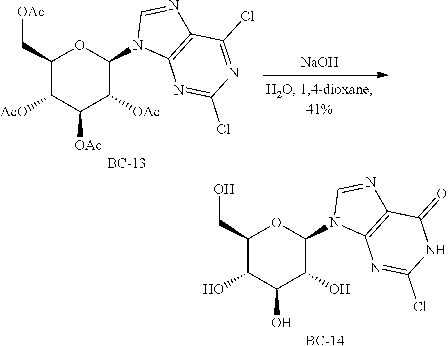

Compound BC-14 was synthesized according to a previously reported procedure. A suspension containing BC-13 (2.1 g, 4.05 mmol, 1.00 equiv.), NaOH (1.62 g, 40.5 mmol, 10 equiv.), H₂O (50 mL), and 1,4-dioxane (20 mL) was heated at 100° C. for 4 hr. The resulting dark red solution was neutralized by the addition of AcOH and then concentrated to dryness in vacuo. Flash column chromatography on C18 using a gradient of 0-50% ACN in H₂O (w/ 0.1% AcOH) afforded BC-14 (560 mg, 41%) as a yellow solid. ¹H NMR (600 MHz, DMSO-d₆): δ 8.30 (s, 1H), 5.37 (d, J=4.1 Hz, 1H), 5.30 (d, J=9.3 Hz, 1H), 5.30-5.23 (m, 1H), 5.20-5.08 (m, 1H), 4.65-4.51 (m, 1H), 3.89-3.83 (m, 1H), 3.70 (d, J=11.4 Hz, 1H), 3.46-3.34 (m, 3H), 3.24 (t, J=9.1 Hz, 1H). Reference: MODERNA THERAPEUTICS INC—WO2017/66793, 2017, A1

### Example 15. Step 2. N⁹—(β-glucopyranosyl)-N²-methylguanine (BC-15, mgglu #3)

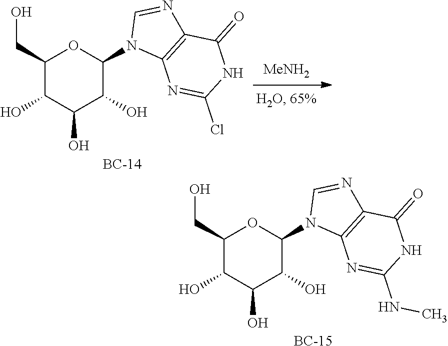

A solution containing BC-14 (330 mg, 0.99 mmol, 1.00 equiv.) and MeNH₂ (12 mL, 40% in H₂O, 154 mmol, 156 equiv.) was heated at 100° C. in a sealed container for 15 hr. The resulting solution was acidified to pH ˜5 w/ AcOH, transferred to a round-bottom flask, and then concentrated to dryness in vacuo. Flash column chromatography on C18 using a gradient of 0%-30% ACN in H₂O (w/ 0.1% AcOH) afforded BC-15 (mgglu #3, 210 mg, 65%) as an off-white solid. mgglu #3 was found to be identical to the corresponding peak using HILIC-MS (Method C) in C. elegans wildtype (N₂) samples by HPLC-HRMS (see FIG. S1b for co-elution data). ¹H NMR (600 MHz, DMSO-d₆): δ 10.67 (br s, 1H), 7.83 (s, 1H), 6.36-6.32 (m, 1H), 5.32 (d, J=4.8 Hz, 1H), 5.26-5.19 (m, 1H), 5.20 (d, J=9.2 Hz, 1H), 5.08 (d, J=4.3 Hz, 1H), 4.59 (t, J=5.8 Hz, 1H), 3.83 (td, J=9.3, 3.8 Hz, 1H), 3.70 (dd, J=11.9, 4.1 Hz, 1H), 3.46-3.40 (m, 1H), 3.39-3.31 (m, 2H), 3.23-3.18 (m, 1H), 2.82 (d, J=4.7 Hz, 3H). ¹³C (126 MHz, DMSO-d₆): δ 157.0, 153.4, 151.3, 135.7, 116.3, 82.2, 80.0, 77.3, 71.4, 69.8, 61.0, 27.5.

HRMS (ESI) m/z: [M+Na]⁺ calcd for C₁₂H₁₇N₅O₆Na⁺ 350.1071; found 350.1053.

### Example 16. Step 1. N⁹—(β-glucopyranosyl)-N², N²-dimethylguanine (BC-16, dmgglu #3)

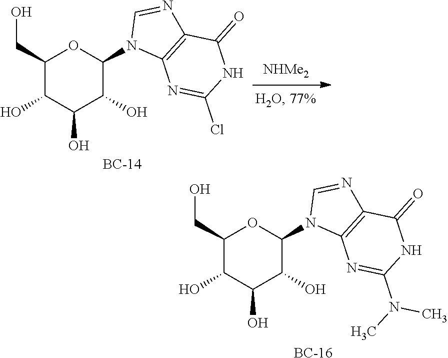

To BC-14 (60 mg, 0.181 mmol, 1.00 equiv.) in a high-pressure flask was added 40% NHMe2 in H₂O (2.5 mL). The flask was sealed and heated to 100° C., at which the resulting solution was stirred for 14 hr. The solution was transferred to a round-bottom flask and concentrated to dryness in vacuo. Flash column chromatography on C18 using a gradient of 0-100% ACN (w/0.1% formic acid) in H₂O (w/ 0.1% formic acid), followed by additional purification with flash column chromatography on silica using a gradient of 20-60% MeOH in DCM afforded BC-16 (48 mg, 77%) as a white solid. dmgglu #3 (BC-16) was compared to isomer peaks using HILIC-MS (Method C) in C. elegans and C. briggsae wildtype samples by HILIC-HRMS (see Figure S1c). ¹H NMR (500 MHz, DMSO-d₆): δ 10.68 (br s, 1H), 7.85 (s, 1H), 5.53-5.23 (m, 2H), 5.20 (d, J=9.2 Hz, 1H), 5.18 (br s, 1H), 4.61 (br s, 1H), 3.85 (t, J=9.1 Hz, 1H), 3.69 (d, J=12.0, 1H), 3.42 (dd, J=12.0, 6.1 Hz, 1H), 3.36-3.30 (m, 3H), 3.20 (t, J=9.2 Hz, 1H), 3.07 (s, 6H). ¹³C NMR (126 MHz, DMSO-d₆): 157.5, 153.0, 151.1, 136.3, 115.6, 82.3, 79.9, 77.2, 71.3, 69.8, 60.9, 37.6. HRMS (ESI) m/z: [M+Na]⁺ calcd for C₁₃H₁₉N₅O₆Na⁺ 364.1227; found 364.1218.

### Example 17. Step 1. Compound BC-18

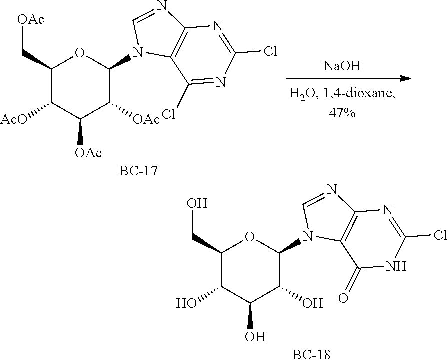

A solution containing BC-17 (515 mg, 0.99 mmol, 1.00 equiv.), NaOH (400 mg, 10.0 mmol, 10.10 equiv.), H₂O (13 mL) and 1,4-dioxane (5 mL) was heated at 100° C. for 4 hr. The dark red solution was cooled to 0° C., acidified to pH=4 by the addition of AcOH, and concentrated to dryness in vacuo. Flash column chromatography on C18 using a gradient of 0%-50% ACN in H₂O (w/ 0.1% formic acid) afforded BC-18 (157 mg, 47%) as a light brown solid. 1H NMR (600 MHz, DMSO-d₆): δ 8.47 (s, 1H), 5.68 (d, J=9.4 Hz, 1H), 5.38 (br s, 1H), 5.28 (br s, 1H), 5.11 (br s, 1H), 4.58-4.50 (m, 1H), 3.84 (d, J=9.2 Hz, 1H), 3.69 (d, J=11.7 Hz, 1H), 3.47-3.42 (m, 1H), 3.37-3.30 (m, 3H), 3.26 (dd, J=9.7, 3.5 Hz, 1H). ¹³C NMR (126 MHz, DMSO-d₆): 163.1, 156.7, 154.5, 143.4, 113.8, 85.0, 80.1, 77.0, 72.0, 69.5, 60.9. HRMS (ESI) m/z: [M+Na]⁺ calcd for C₁₁H₁₃ClN₄O₆Na⁺ 355.0416; found 355.0410. The compound was primarily detected as its in-source fragment: [M+H]⁺ calcd for C₅H₄ClN₄O⁺171.0068; found 171.0066.

### Example 17. Step 2. N⁷-(β-glucopyranosyl)-N²,N²-dimethylguanine, dmgglu #1 (BC-19)

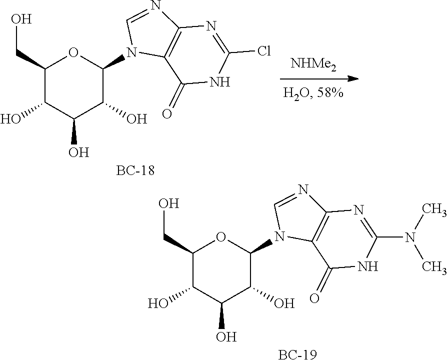

To BC-18 (51 mg, 0.154 mmol, 1.00 equiv.) in a high-pressure flask was added 40% NHMe2 in H₂O (5.1 mL). The flask was sealed and heated to 100° C., at which the resulting solution was stirred for 19 hr. The solution was cooled to room temp., transferred to a round-bottom flask, neutralized with AcOH, and concentrated to dryness in vacuo. Flash column chromatography on C18 using a gradient of 0-100% ACN in H₂O (w/ 0.1% formic acid) afforded dmgglu #1 (BC-19, 30 mg, 58%) as an off-white solid. dmgglu #3 was found to be identical to the corresponding peak using HILIC-MS (Method C) in C. elegans wildtype (N₂) samples by HPLC-HRMS (see Figure Slc for co-elution data). ¹H NMR (500 MHz, DMSO-d₆): 10.84 (br s, 1H), 8.20 (s, 1H), 5.57 (d, J=9.3 Hz, 1H), 5.34 (br s, 1H), 5.25 (br s, 1H), 5.09 (br s, 1H), 4.53 (t, J=6.1 Hz, 1H), 3.84 (t, J=9.2 Hz, 1H), 3.68 (d, J=12.0 Hz, 1H), 3.47-3.28 (m, 3H), 3.24 (t, J=9.3 Hz, 1H), 3.03 (s, 6H). ¹³C NMR (126 MHz, DMSO-d₆): δ 159.5, 154.9, 152.7, 142.3, 107.5, 84.7, 79.8, 77.2, 71.8, 69.5, 60.9, 37.9. HRMS (ESI) m/z: [M+Na]⁺ calcd for C₁₃H₁₉N₅O₆Na⁺ 364.1227; found 364.1217. A large fraction of the sample was detected as the in-source fragment: [M+H]⁺ calcd for C₇H₁₀N₅O⁺ 180.0880; found 180.0877.

### Example 18. Step 1. mgglu #1 (BC-21) and mgglu #5 (BC-22)

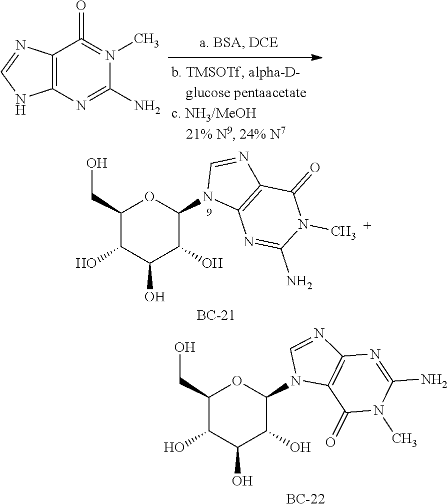

Under Ar, a suspension of N¹-methylguanine (BC-20, 750 mg, 4.54 mmol, 1.36 equiv.), N,O-bis(trimethylsilyl)acetamide (2.4 mL, 9.84 mmol, 3.02 equiv.) and DCE (20 mL) was refluxed for 1 hr until the solution was homogeneous. After cooling to room temp., TMSOTf (1.35 mL, 7.48 mmol, 2.29 equiv.) and alpha-D-glucose pentaacetate (1.27 g, 3.26 mmol, 1.00 equiv.) were added and the resulting solution was refluxed for 36 hr. The resulting orange solution was then concentrated to dryness in vacuo, followed by the addition of NH₃/MeOH (7N, 38 mL, 266 mmol, 81 equiv.). The resulting solution was stirred for 7 hr at room temp. and concentrated to dryness in vacuo. Flash column chromatography on C18 using a gradient of 0%-100% ACN in H₂O (w/ 0.1% AcOH) was first performed, of which fractions containing products BC-21 and BC-22 and 1-methylguanine (BC-20) were collected (elution at ˜10% ACN). The dried mixture was dissolved in MeOH and filtered, followed by concentration in vacuo with 15 g of silica gel for dry-loading. Flash column chromatography on silica using a gradient of 35%-100% MeOH in DCM was then performed, which afforded BC-22 (256 mg, 24%) and BC-21 (219 mg, 21%), of which could be mostly separated with subsequent chromatography. mgglu #1 (BC-21) and mgglu #5 (BC-22) were compared to isomer peaks (m/z=350.1071) using HILIC-MS in C. elegans wildtype (N₂) samples by HILIC-HRMS (Method C) and MS² (see Figure Slb and S6 for co-elution and MS² data, respectively). 1H NMR (N⁹-isomer, BC-21) (600 MHz, D₂O): δ 7.98 (s, 1H), 5.47 (d, J=9.4 Hz, 1H), 4.16 (t, J=9.0 Hz, 1H), 3.93 (dd, J=12.4, 1.7 Hz, 1H), 3.82 (dd, J=12.3, 5.0 Hz, 1H), 3.74 (dd, J=9.7, 1.8 Hz, 1H), 3.71 (t, J=8.9 Hz, 1H), 3.66 (t, J=9.3 Hz, 1H), 3.41 (s, 3H). ¹³C NMR (N⁹-isomer, BC-21) (126 MHz, D₂O): δ 159.2, 155.4, 150.1, 138.6, 116.4, 83.4, 79.3, 76.9, 71.8, 69.6, 61.0, 29.2. HRMS (ESI) m/z: [M+Na]⁺ calcd for C12H17N₅O₆Na⁺ 350.1071; found 350.1060. ¹H NMR (N⁷-isomer, BC-22) (600 MHz, D₂O): δ 8.29 (s, 1H), 5.79 (d, J=9.2 Hz, 1H), 4.18 (t, J=9.0 Hz, 1H), 3.94 (dd, J=12.4, 1.7 Hz, 1H), 3.80 (dd, J=12.4, 5.5 Hz, 1H), 3.74 (dd, J=9.5, 1.7 Hz, 1H), 3.71 (t, J=8.9 Hz, 1H), 3.66 (t, J=9.3 Hz, 1H), 3.47 (s, 3H). ¹³C NMR (N⁷-isomer, BC-22) (126 MHz, D₂O): δ 158.1, 155.9, 155.2, 144.6, 108.2, 85.7, 79.3, 76.8, 72.7, 69.7, 61.2, 29.1. HRMS (ESI) m/z: [M+Na]⁺ calcd for C₁₂H₁₇N₅O₆Na⁺ 350.1071; found 350.1053.

### Example 18. Step 2. 6′-O, 4′-O-TIPDSi-N⁷—(β-glucopyranosyl)-N¹-methylguanine (BC-23)

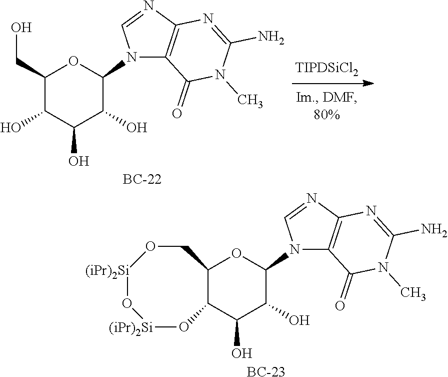

To a solution of BC-22 (120 mg, 0.367 mmol, 1.00 equiv.) in DMF (2 mL) at 0° C. was added TIPDSiCl₂ (175 μL, 0.504 mmol, 1.50 equiv.) and imidazole (104 mg, 1.53 mmol, 4.55 equiv.). The reaction mixture was stirred for 15 min at 0° C. and then diluted with DCM, followed by addition of H₂O. Organics were extracted 4× with DCM, combined, and then basified using sat. aq. NaHCO₃. Organics were then extracted from the aq. layer 3× with DCM, dried using MgSO₄, filtered, and concentrated in vacuo. Flash column chromatography on silica using a gradient of 5-50% MeOH in DCM was performed, affording BC-23 (168 mg, 80%) as a white solid. 1H NMR (500 MHz, methanol-d₄): δ 8.17 (s, 1H), 5.79 (d, J=9.4 Hz, 1H), 4.16 (dd, J=12.8, 2.1 Hz, 1H), 3.99 (t, J=9.0 Hz, 1H), 3.95 (t, J=9.0 Hz, 1H), 3.90 (dd, J=12.8, 1.0 Hz, 1H), 3.60 (t, J=9.0 Hz, 1H), 3.49 (dt, J=9.5, 1.6 Hz, 1H), 3.45 (s, 3H), 1.25-0.96 (m, 28H). ¹³C NMR (126 MHz, methanol-d₄): δ 158.8, 155.8, 143.3, 136.3, 109.1, 86.7, 80.7, 78.4, 74.0, 70.4, 62. 1, 28.7, 18.0, 17.8, 17.7, 17.6, 14.9, 14.5, 14.0, 13.8.

### Example 18. Step 3. Compounds BC-24 and BC-25

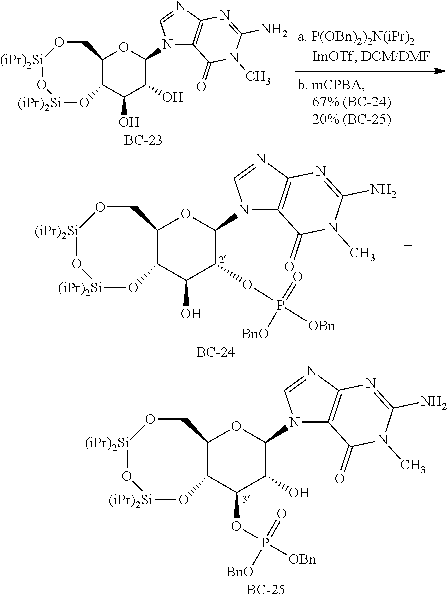

To BC-23 (153 mg, 0.27 mmol, 1.00 equiv.) in DCM (2 mL) and DMF (1 mL) was added dibenzyl N,N-diisopropylphosphoramidite (0.36 mL, 1.07 mmol, 3.96 equiv.), and ImOTf (277 mg, 1.27 mmol, 4.70 equiv.) incrementally over a 2 hr period. Note: this was done to ensure minimization of bis-phosphitylation products. The reaction mixture was then cooled to −78° C. after which mCPBA (77% max, 165 mg, 0.74 mmol, 2.73 equiv.) was added. The resulting mixture was stirred at −78° C. for 20 min. and was then quenched with the addition of sat. aq. NaHCO₃ (10 mL) followed by addition of DCM (20 mL). The organic layer was collected and the aqueous layer was extracted an additional 2× with DCM (20 mL each). Combined organics were dried with MgSO₄, filtered, and concentrated in vacuo. Flash column chromatography on silica using a gradient of 5-40% MeOH in DCM was performed, affording separable BC-24 (149 mg, 67%) and BC-25 (44 mg, 20%) as white solids. 2′-O isomer (BC-24): ¹H NMR (500 MHz, methanol-d₄): δ 8.21 (br s, 1H), 7.36-7.13 (m, 8H), 7.14-6.97 (m, 2H), 6.24-5.63 (m, 1H), 5.04-4.89 (m, 2H), 4.78-4.50 (m, 3H), 4.16 (d, J=12.6 Hz, 1H), 4.11-3.95 (m, 1H), 3.91 (d, J=12.6 Hz, 1H), 3.88-3.77 (m, 1H), 3.59-3.47 (m, 1H), 3.25 (br s, 3H), 1.30-0.93 (m, 28H). 2′-O isomer (BC-24): ¹³C NMR (126 MHz, methanol-d₄): δ 156.8, 137.1, 136.8, 129.5, 129.4, 128.9, 109.1, 80.8, 76.9, 70.9, 70.4, 61.9, 28.6, 18.1, 17.8, 17.7, 17.6, 14.8, 14.5, 14.0, 13.8. 3′-O isomer (BC-25): 1H NMR (500 MHz, methanol-d₄): δ 8.19 (s, 1H), 7.39-7.28 (m, 10H), 5.77 (d, J=8.5 Hz, 1H), 5.16-5.06 (m, 2H), 5.08-4.96 (m, 2H), 4.54-4.40 (m, 2H), 4.20 (t, J=9.0 Hz, 1H), 4.14 (dd, J=12.8, 1.4 Hz, 1H), 3.93 (dd, J=12.7, 1.7 Hz, 1H), 3.52 (d, J=9.2 Hz, 1H), 3.46 (s, 3H), 1.19-0.82 (m, 28H). 3′-O isomer (BC-25): ¹³C NMR (126 MHz, methanol-d₄): δ 159.0, 155.9, 155.6, 144.0, 137.4 (J=7.0 Hz), 137.2 (d, J=7.4 Hz), 129.7, 129.6, 129.3, 129.0, 109.0, 87.0, 85.6 (d, J=6.8 Hz), 80.1, 72.7, 71.1 (d, J=5.6 Hz), 70.8 (d, J=5.3 Hz), 69.6 (d, J=5.6 Hz), 62.0, 28.7, 18.0, 17.9, 17.8, 17.7, 17.5, 14.4, 14.3, 14.2, 14.1. Note: 2′-O dibenzyl phosphate isomer (BC-24) exhibited extreme line broadening for several signals.

### Example 18. Step 4. Compounds BC-26 and BC-27

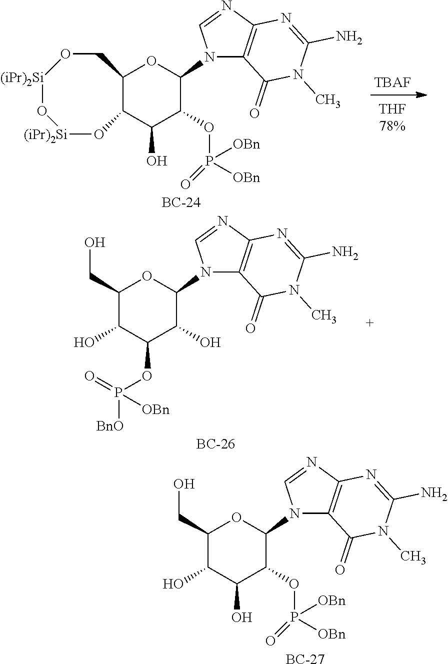

To a solution of BC-24 (90 mg, 0.108 mmol, 1.00 equiv.) in THF (3 mL) at 0° C. was added TBAF (1M in THF, 275 μL, 0.27 mmol, 2.50 equiv.). After 15 min., AcOH (75 uL) was added, and the resulting solution was concentrated in vacuo. Flash column chromatography on silica using a gradient of 5-40% MeOH in DCM was performed, affording BC-26 (37.5 mg, 0.036 mmol, 59%) and BC-27 (11.5 mg, 8 mmol, 18%) of which were mostly separable. 3′-O isomer (BC-26): ¹H NMR (500 MHz, methanol-d₄): δ 8.23 (s, 1H), 7.39-7.21 (m, 1OH), 5.78 (d, J=9.1 Hz, 1H), 5.15 (d, J=7.5 Hz, 2H), 5.13-5.10 (m, 2H), 4.49 (td, J=9.1, 8.1 Hz, 1H), 4.36 (t, J=9.2 Hz, 1H), 3.90 (dd, J=12.2, 2.3 Hz, 1H), 3.84 (t, J=9.5 Hz, 1H), 3.77 (dd, J=12.2, 5.2 Hz, 1H), 3.63 (ddd, J=9.9, 5.2, 2.3 Hz, 1H), 3.47 (s, 3H). 3′-O isomer (BC-26): ¹³C NMR (126 MHz, methanol-d₄): δ 159.3, 155.9, 155.8, 144.4, 137.5 (d, J=3.5 Hz), 137.4 (d, J=3.5 Hz), 129.6, 129.5, 129.1, 129.0, 109.0, 86.8, 86.0 (d, J=6.9 Hz), 80.5, 72.7 (d, J=3.5 Hz), 70.8 (d, J=5.8 Hz), 69.7 (d, J=3.2 Hz), 62.2, 28.8. 2′-O isomer (BC-27): ¹H NMR (600 MHz, methanol-d₄): δ 8.28 (s, 1H), 7.40-6.97 (m, 10H), 6.17-5.86 (m, 1H), 5.00-4.93 (m, 1H), 4.67 (dd, J=12.3, 6.7 Hz, 1H), 4.65-4.52 (m, 1H), 3.89 (dd, J=12.2, 1.9 Hz, 1H), 3.78 (t, J=9.0 Hz, 1H), 3.73 (dd, J=12.2, 5.6 Hz, 1H), 3.64 (t, J=9.5 Hz, 1H), 3.63 (ddd, J=9.6, 5.6, 2.0 Hz, 1H), 3.27 (s, 3H).

### Example 18. Step 5. Compound BC-26

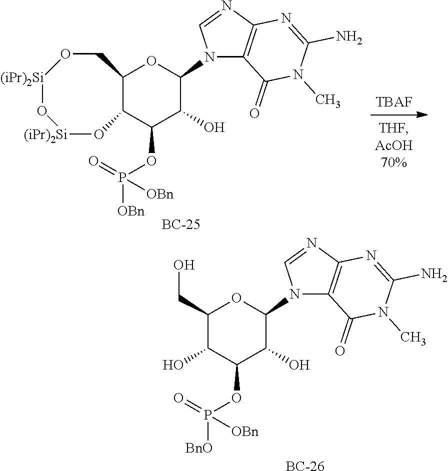

To a solution of BC-25 (65 mg, 0.078 mmol, 1.00 equiv.) in THF (3 mL) containing AcOH (20 uL) at 0° C. was added TBAF (1M in THF, 200 μL, 0.20 mmol, 2.56 equiv.). The solution was slowly warmed to RT over a 4 hr period, then additional AcOH (40 uL) was added, and the reaction mixture was concentrated in vacuo. Flash column chromatography on silica using a 20 gradient of 15-50% MeOH in DCM was performed, afforded BC-26 (32 mg, 70%).

### Example 18. Step 6. mgglu #6 (BC-28)

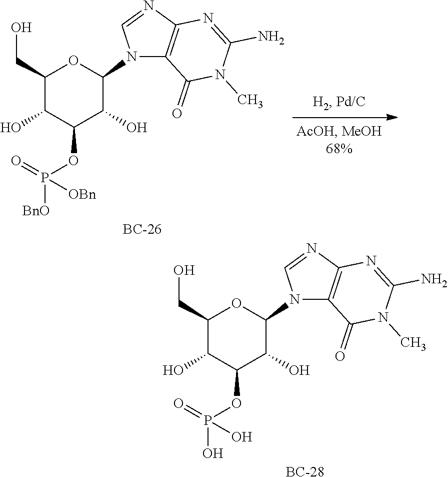 [SMILES: Cn1c(N)nc2ncn([C@@H]3O[C@H](CO)[C@@H](O)[C@H](OP(=O)(OCc4ccccc4)OCc4ccccc4)[C@H]3O)c2c1=O ; "BC-26" ; Cn1c(N)nc2ncn([C@@H]3O[C@H](CO)[C@@H](O)[C@H](OP(=O)(O)O)[C@H]3O)c2c1=O ; "BC-28"]

A suspension containing BC-26 (17 mg, 0.020 mmol, 1.00 equiv.), Pd/C (35 mg, 10% w/w), AcOH (300 μL) and MeOH (3 mL) was sparged with Ar for 5 min. then switched to H₂, through which the suspension was rapidly stirred under for 4 hr. After sparging with Ar, the reaction mixture was filtered through celite, washed with MeOH/H₂O, and the collected filtrate was concentrated to dryness in vacuo affording mgglu #6 (BC-28, 8 mg, 68%) at 92% purity. mgglu #6 was found to be identical to the major isomer peak in C. elegans wildtype (N₂) endo-metabolome samples by HILIC-HRMS (see Figures S2c and S8 for co-elution and MS² data, respectively). Chromatographic Method E was used. mgglu #6-HRMS (ESI) m/z: [M+H]⁺ calcd for C₁₂H₁₉N₅O₉P⁺ 408.0915; found 408.0914.

### Example 19. Step 1. Compound BC-29

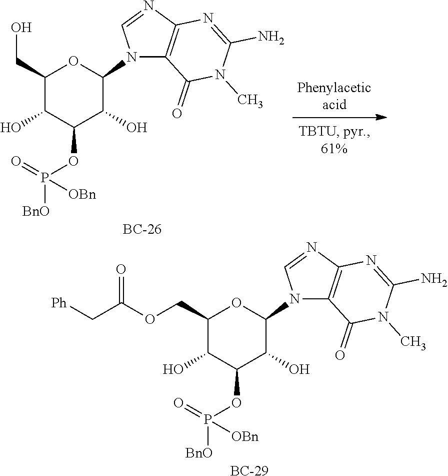

Phenylacetic acid (8 mg, 0.059 mmol, 4.21 equiv.) and TBTU (19 mg, 0.059 mmol, 4.21 equiv.) were added to a solution of BC-26 (8.2 mg, 0.014 mmol, 1.00 equiv.) in 1 mL dry pyridine. The resulting mixture was stirred for 4 hr at room temp., MeOH (1 mL) was added, transferred to a round-bottom flask, and then concentrated to dryness in vacuo. Flash column chromatography on silica using a gradient of 2.5-40% MeOH in DCM was performed, affording BC-29 (6.0 mg, 61%) as a white solid. Note: co-eluting HOBt was separated by subsequent chromatography. ¹H NMR (500 MHz, methanol-d₄): δ 8.10 (s, 1H), 7.40-7.15 (m, 15H), 5.73 (d, J=9.1 Hz, 1H), 5.17-5.08 (m, 4H), 4.54 (dd, J=12.1, 1.8 Hz, 1H), 4.47 (q, J=8.7 Hz, 1H), 4.37 (t, J=9.1 Hz, 1H), 4.29 (dd, J=12.1, 4.9 Hz, 1H), 3.84-3.76 (m, 2H), 3.67 (d, J=15.3 Hz, 1H), 3.63 (d, J=15.3 Hz, 1H), 3.44 (s, 3H). ¹³C NMR (126 MHz, methanol-d₄): δ 173.2, 159.3, 155.9, 155.7, 144.2, 137.5 (d, J=2.6 Hz), 137.4 (d, J=2.5 Hz), 135.6, 130.3, 129.6, 129.5, 129.4, 129.0, 128.0, 108.9, 86.7, 85.7, 85.6 (d, J=7.0 Hz), 77.7, 72.4 (d, J=3.2 Hz), 70.8 (d, J=5.9 Hz), 70.0 (d, J=3.4 Hz), 64.4, 41.8, 28.8.

### Example 19. Step 2. mgglu #51 (BC-30)

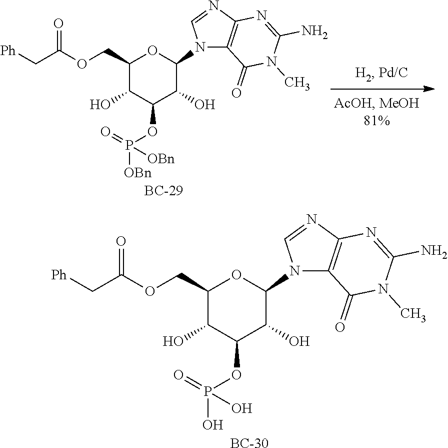

A suspension containing BC-29 (5 mg, 0.0071 mmol, 1.00 equiv.), Pd/C (6.6 mg, 10% w/w), AcOH (47 μL) and MeOH (2 mL) was sparged with Ar for 5 min. then switched to H₂, through which the suspension was rapidly stirred under for 1.5 hr. After sparging with Ar, the reaction mixture was filtered through celite using MeOH/H₂O and the collected filtrate was concentrated to dryness in vacuo, affording mgglu #51 (BC-30, 3.0 mg, 81%) which was deemed pure enough for no further purification steps. mgglu #51 was found to be identical to the major isomer peak on C₁₈ in C. elegans wildtype (N₂),fem-3 (gf), and him-5 endo-metabolome samples by HPLC-HRMS (Method B) and MS² (see Figures S3c and S10 for co-elution and MS² data, respectively). mgglu #51—HRMS (ESI) m/z: [M+H]⁺ calcd for C₂₀H₂₅N₅O₁₀P⁺ 526.1333; found 526.1332.

### Example 20. Step 1. Compound BC-31

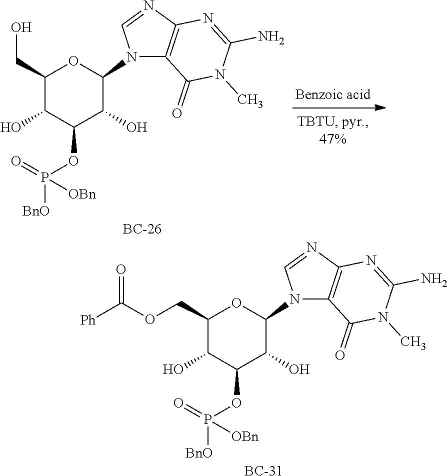

Benzoic acid (22 mg, 0.18 mmol, 9.49 equiv.) and TBTU (55 mg, 0.14 mmol, 7.64 equiv.) were added to a solution of BC-26 (11 mg, 0.019 mmol, 1.00 equiv.) in 1.5 mL dry pyridine. The resulting mixture was stirred for 10 hr at room temp., MeOH (2 mL) was added, transferred to a round-bottom flask, then concentrated to dryness in vacuo. Flash column chromatography on silica using a gradient of 5-40% MeOH in DCM was performed, affording BC-31 (6.0 mg, 47%) as a white solid. Note: co-eluting HOBt was separated by subsequent chromatography. ¹H NMR (500 MHz, methanol-d₄): δ 8.19 (s, 1H), 8.06 (d, J=7.2 Hz, 2H), 7.60 (t, J=7.4 Hz, 1H), 7.47 (t, J=7.8 Hz, 2H), 7.39-7.19 (m, 10H), 5.79-5.69 (m, 1H), 5.16 (d, J=7.4 Hz, 2H), 5.14-5.10 (m, 2H), 4.70 (dd, J=12.1, 1.7 Hz, 1H), 4.62-4.46 (m, 3H), 4.03-3.91 (m, 2H), 3.40 (s, 3H). ¹³C NMR (126 MHz, methanol-d₄): δ 167.8, 159.4, 155.9, 155.5, 144.4, 137.5 (d, J=3.1 Hz), 137.4 (d, J=3.3 Hz), 134.3, 131.3, 130.8, 129.6, 129.50, 129.4, 129.0, 108.9, 87.1, 85.7 (d, J=7.1 Hz), 77.8, 72.2 (d, J=3.5 Hz), 70.8 (d, J=6.0 Hz), 69.9 (d, J=3.4 Hz), 64.6, 28.8.

### Example 20. Step 2. mgglu #52 (BC-32)

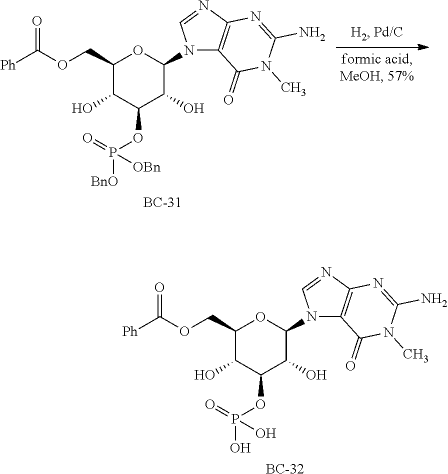

A suspension containing BC-31 (6.0 mg, 0.0087 mmol, 1.00 equiv.), Pd/C (14 mg, 10% w/w), formic acid (50 μL) and MeOH (2 mL) was sparged with Ar for 5 min. then switched to H₂, through which the suspension was rapidly stirred under for 3 hr. After sparging with Ar, the reaction mixture was filtered through celite using MeOH/H₂O and the collected filtrate was concentrated to dryness in vacuo, affording mgglu #52 (BC-32, 2.5 mg, 57%) which was deemed pure enough for no further purification steps. mgglu #52 was found to be identical to the major isomer peak on C18 in C. elegans wildtype (N₂) by HPLC-HRMS (Method A) and MS² (see Figures S4 and S12 for co-elution and MS² data, respectively). mgglu #52—HRMS (ESI) m/z: [M+H]⁺ calcd for C₁₉H₂₂N₅O₁₀P⁺ 512.1177; found 512.1158.

### Example 21. Step 1. 6′-O, 4′-O-TIPDSi-N⁹—(β-glucopyranosyl)-N¹-methylguanine (BC-33)

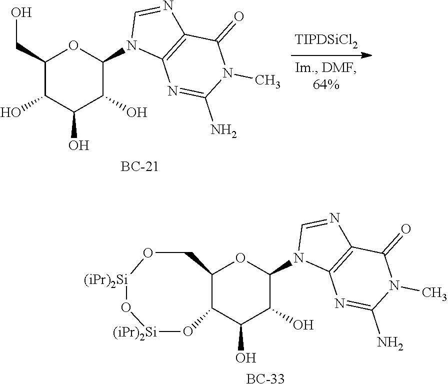

To a solution of BC-21 (70 mg, 0.214 mmol, 1.00 equiv.) in DMF (2 mL) at 0° C. was added TIPDSiCl₂ (120 μL, 0.376 mmol, 1.75 equiv.) and imidazole (66 mg, 0.970 mmol, 4.53 equiv.). The reaction mixture was stirred for 45 min at 0° C. and then diluted with DCM, followed by addition of H₂O. Organics were extracted 3× with DCM, combined, and then basified using sat. aq. NaHCO₃. Organics were then extracted from the aq. layer 3× with DCM, dried using MgSO₄, filtered, and concentrated in vacuo. Flash column chromatography on silica using a gradient of 5-40% MeOH in DCM was performed, affording BC-33 (77 mg, 64%) as a white solid. ¹H NMR (500 MHz, methanol-d₄): δ 7.86 (s, 1H), 5.40 (d, J=9.4 Hz, 1H), 4.16 (dd, J=12.7, 2.2 Hz, 1H), 3.98-3.88 (m, 3H), 3.59 (t, J=9.0 Hz, 1H), 3.47 (dt, J=9.5, 1.5 Hz, 1H), 3.46 (s, 3H), 1.25-0.96 (m, 28H). ¹³C NMR (126 MHz, methanol-d₄): δ 158.9, 156.2, 151.4, 137.6, 116.8, 84.3, 80.8, 78.3, 73.7, 70.4, 62.1, 28.8, 18.0, 17.8, 17.8, 17.8, 17.7, 17.7, 17.6, 14.9, 14.5, 14.0, 13.8.

### Example 21. Step 2. Compound BC-34

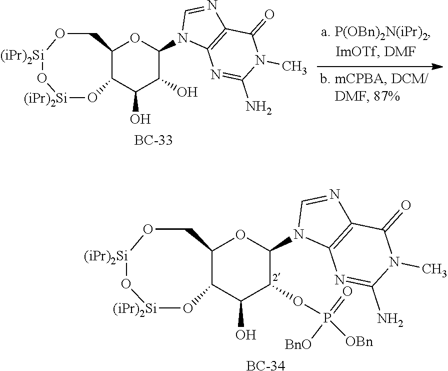

To a suspension of BC-33 (74 mg, 0.130 mmol, 1.00 equiv.) and DCM (1 mL)/DMF (3 mL) at room temp. was added dibenzyl N,N-diisopropylphosphoramidite (0.13 mL, 0.39 mmol, 3.00 equiv.), and ImOTf (85 mg, 0.39 mmol, 3.00 equiv.). The reaction mixture was stirred at room temp. for 15 min. at which a homogenous solution formed and then cooled to −78° C. after which 1 mL of DCM and mCPBA (77% max, 87 mg, 0.39 mmol, 3.00 equiv.) were added. The resulting mixture was stirred at −78° C. for 10 min. and was then quenched with the addition of sat. aq. NaHCO₃ (3 mL) followed by the addition of H₂O (10 mL) and DCM (20 mL). The organic layer was collected and the aqueous layer was extracted 3× with DCM (20 mL each). Combined organics were dried with MgSO₄, filtered, and concentrated in vacuo. Flash column chromatography on silica using a gradient of 2.5-20% MeOH in DCM was performed, affording BC-34 (94 mg, 87%). ¹H NMR (600 MHz, methanol-d₄): δ 7.88 (s, 1H), 7.33-7.20 (m, 8H), 7.08-7.02 (m, 2H), 5.68 (d, J=9.3 Hz, 1H), 4.93 (dd, J=11.5, 7.5 Hz, 1H), 4.87 (dd, J=11.1, 8.3 Hz, 1H), 4.68 (dd, J=11.7, 6.2 Hz, 1H), 4.58 (dd, J=11.7, 8.3 Hz, 1H), 4.17 (dd, J=12.8, 2.1 Hz, 1H), 3.99 (t, J=9.2 Hz, 1H), 3.93 (dd, J=12.9, 1.5 Hz, 1H), 3.87 (t, J=9.0 Hz, 1H), 3.54 (dt, J=9.5, 1.5 Hz, 1H), 3.26 (s, 3H), 1.27-0.97 (m, 28H). ¹³C NMR (126 MHz, methanol-d₄): δ 158.6, 156.1, 151.3, 137.6, 137.1 (d, J=7.1 Hz), 136.7 (d, J=7.1 Hz), 129.5, 129.4, 128.9, 128.3, 116.8, 82.3, 80.9, 80.1, 76.9, 70.9 (d, J=5.9 Hz), 70.6, 70.3 (d, J=5.3 Hz), 61.9, 28.8, 18.0, 17.8, 17.7, 17.6, 14.8, 14.5, 14.1, 13.8.

### Example 21. Step 3. Compounds BC-35 and BC-36

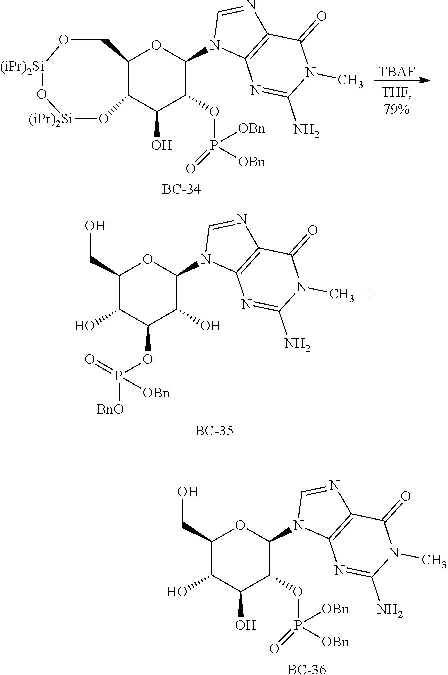

To a solution of BC-34 (96 mg, 0.116 mmol, 1.00 equiv.) in THF (4 mL) at 0° C. was added TBAF (1M in THF, 300 μL, 0.30 mmol, 2.59 equiv.). After 10 min., AcOH (100 μL) was added, and the resulting solution was concentrated in vacuo. Due to poor solubility of the resulting 3′-O product in DCM/MeOH, the crude was dissolved in a mixture of ACN and minimal H₂O and dry-loaded with 2 gram silica. Flash column chromatography on silica using a gradient of 5-40% MeOH in DCM was performed, affording pure 3′-O isomer (BC-35, 34 mg, 50%) and several mixed fractions containing 4 mg of 3′-O isomer (6%) and 16 mg of 2′-O isomer (BC-36, 23%). 2′-O isomer (BC-36): ¹H NMR (500 MHz, methanol-d₄): δ 7.99 (s, 1H), 7.38-7.20 (m, 8H), 7.10-6.99 (m, 2H), 5.69 (d, J=9.3 Hz, 1H), 4.97 (dd, J=11.8, 7.4 Hz, 1H), 4.92 (dd, J=11.9, 8.2 Hz, 1H), 4.66 (dd, J=11.9, 6.2 Hz, 1H), 4.53 (dd, J=11.9, 7.9 Hz, 1H), 3.89 (dd, J=12.4, 1.8 Hz, 1H), 3.80 (d, J=8.7 Hz, 1H), 3.74 (dd, J=12.1, 4.8 Hz, 1H), 3.65-3.51 (m, 2H), 3.26 (s, 3H). 2′-O isomer (BC-36): ¹³C NMR (126 MHz, methanol-d₄): δ 158.6, 156.2, 151.3, 137.1 (d, J=7.3 Hz), 136.7 (d, J=7.8 Hz), 129.6, 129.5, 129.3, 128.9, 128.1, 82.0, 81.3, 79.8, 77.3, 71.2, 70.9 (d, J=5.9 Hz, 1H), 70.21 (d, J=5.8 Hz, 1H), 62.2, 28.8. 3′-O isomer (BC-35): ¹H NMR (500 MHz, DMSO-d₆): δ 7.94 (s, 1H), 7.43-7.28 (m, 8H), 7.09 (s, 2H), 5.87 (d, J=6.6 Hz, 1H), 5.60 (d, J=7.3 Hz, 1H), 5.30 (d, J=9.3 Hz, 1H), 5.12 (d, J=6.9 Hz, 2H), 5.10-5.03 (m, 2H), 4.74 (t, J=5.9 Hz, 1H), 4.39 (q, J=8.9 Hz, 1H), 4.23 (td, J=9.2, 6.5 Hz, 1H), 3.72 (dd, J=11.2, 5.7 Hz, 1H), 3.60-3.47 (m, 2H), 3.43 (ddd, J=11.2, 5.8, 1.8 Hz, 1H), 3.32 (s, 3H). 3′-O isomer (BC-35): ¹³C NMR (126 MHz, DMSO-d₆): δ 156.4, 154.3, 149.7, 136.5 (d, J=3.6 Hz), 136.4 (d, J=3.8 Hz), 136.1, 128.4, 128.2, 128.1, 127.7, 115.5, 84.3 (d, J=6.7 Hz), 81.9, 79.4, 69.9, 68.5, 68.4, 68.3, 60.4, 28.1.

### Example 21. Step 4. mgglu #2 (BC-37)

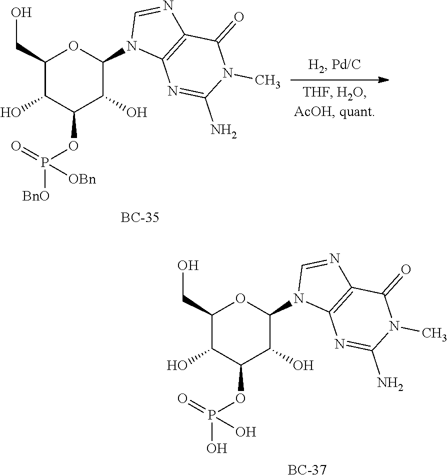 [SMILES: Cn1c(N)nc2c(ncn2[C@@H]2O[C@H](CO)[C@@H](O)[C@H](OP(=O)(OCc3ccccc3)OCc3ccccc3)[C@H]2O)c1=O ; "BC-35" ; Cn1c(N)nc2c(ncn2[C@@H]2O[C@H](CO)[C@@H](O)[C@H](OP(=O)(O)O)[C@H]2O)c1=O ; "BC-37"]

A suspension containing BC-35 (8 mg, mmol, 1.00 equiv.), Pd/C (13 mg, 10% w/w), AcOH (100 μL) H₂O/THF (4 mL, 1:1) was sparged with Ar for 5 min. then switched to H₂, through which the suspension was rapidly stirred under for hr. After sparging with Ar, the reaction mixture was filtered through celite using MeOH/H₂O and the collected filtrate was concentrated to dryness in vacuo affording mgglu #2 (BC-37, 5.5 mg, quant). mgglu #X was compared to the corresponding peak in C. elegans wildtype (N₂) endo-metabolome samples by HILIC-HRMS (see Figure S2c for co-elution data). Chromatographic Method E was used.

### Example 22. Step 1. Compound BC-38

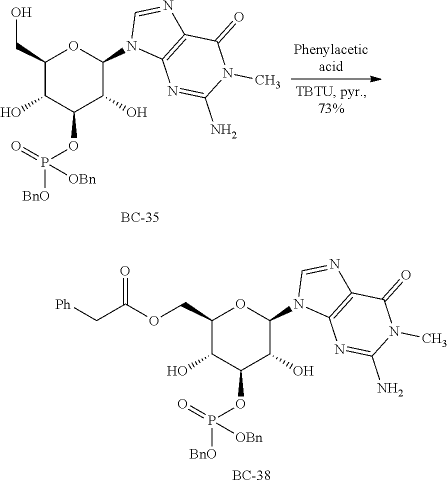

Phenylacetic acid (9.0 mg, 0.066 mmol, 2.64 equiv.) and TBTU (21 mg, 0.065 mmol, 2.60 equiv.) were added to a solution of BC-35 (15 mg, 0.025 mmol, 1.00 equiv.) in 1 mL dry pyridine. The resulting mixture was stirred for 4 hr at room temp., MeOH (1 mL) and DCM (2 mL) was added, the solution was transferred to a round-bottom flask, and then concentrated to dryness in vacuo ensuring all pyridine was removed. Flash column chromatography on silica using a gradient of 2.5-40% MeOH in DCM was performed, affording BC-38 (12.8 mg, 73%) as a white solid. ¹H NMR (500 MHz, methanol-d₄): δ 7.77 (s, 1H), 7.40-7.16 (m, 15H), 5.44 (d, J=9.4 Hz, 1H), 5.17-5.08 (m, 4H), 4.50 (dd, J=12.0, 1.9 Hz, 1H), 4.45 (q, J=8.7 Hz, 1H), 4.29 (dd, J=12.0, 5.6 Hz, 1H), 4.24 (t, J=9.3 Hz, 1H), 3.78 (ddd, J=10.0, 5.6, 2.2 Hz, 1H), 3.71 (t, J=9.4 Hz, 1H), 3.66 (d, J=15.2 Hz, 1H), 3.62 (d, J=15.2 Hz, 1H), 3.46 (s, 3H). ¹³C NMR (126 MHz, methanol-d₄): δ 173.2, 158.9, 156.2, 151.4, 137.8, 137.5 (d, J=2.6 Hz), 137.4 (d, J=2.6 Hz), 135.6, 130.4, 129.6, 129.5, 129.0, 128.0, 116.9, 85.7 (d, J=6.9 Hz), 85.6, 83.9, 77.8, 71.9 (d, J=3.6 Hz), 70.8, 70.1 (d, J=3.6 Hz), 64.4, 41.8, 28.8.

### Example 22. Step 2. mgglu #11 (BC-39)

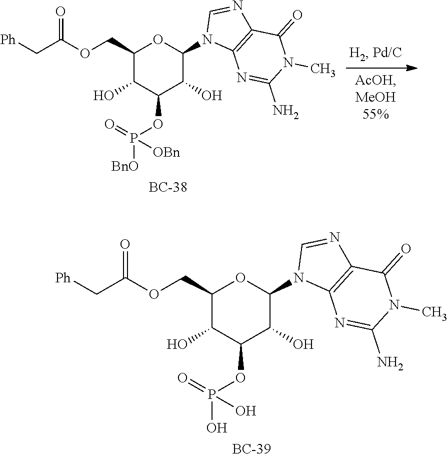

A suspension containing BC-38 (12.5 mg, 0.018 mmol, 1.00 equiv.), Pd/C (17 mg, 10% w/w), AcOH (117 μL) and MeOH (2.5 mL) was sparged with Ar for 5 min. then switched to H₂, through which the suspension was rapidly stirred under for 1.5 hr. After sparging with Ar, the reaction mixture was filtered through celite using MeOH/H₂O and the collected filtrate was concentrated to dryness in vacuo. The dried solution was loaded onto celite in H₂O and flash column chromatography on C18 using a gradient of 0-100% ACN (w/ 0.1% formic acid) in H₂O (w/ 0.1% formic acid) was performed, affording mgglu #11 (BC-39, 5.0 mg, 55%) of which was found to be identical to a minor isomer peak on C18 in C. elegans wildtype (N2) and fem-3 (OE) and him-5 endo-metabolome samples by HPLC-HRMS (see Figure S3c for co-elution data). Chromatographic Method B was used.

### Example 23. Step 1. 6′-O, 4′-O-TIPDSi-N⁹—(β-glucopyranosyl)-N²-methylguanine (BC-40)

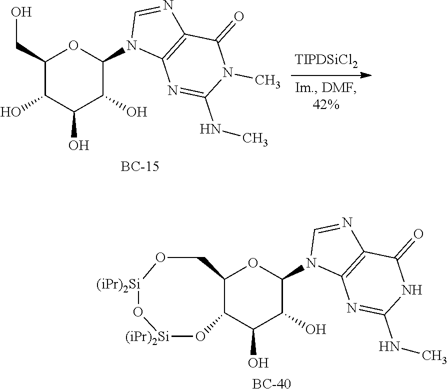

To a solution of BC-15 (95 mg, 0.290 mmol, 1.00 equiv.) in DMF (2 mL) at 0° C. was added TIPDSiCl₂ (140 μL, 0.435 mmol, 1.50 equiv.) and imidazole (93 mg, 1.36 mmol, 4.70 equiv.). The reaction mixture was stirred for 45 min at 0° C. and then diluted with DCM, followed by addition of H₂O. Organics were extracted 3× with DCM, combined, and then basified using sat. aq. NaHCO₃. Organics were then extracted from the aq. layer 3× with DCM, dried using Na₂SO₄, filtered, and concentrated in vacuo. Flash column chromatography on silica using a gradient of 5-40% MeOH in DCM was performed, affording BC-40 (70 mg, 42%) as a white solid. 1H NMR (600 MHz, methanol-d₄): δ 7.84 (s, 1H), 5.42 (d, J=9.3 Hz, 1H), 4.17 (dd, J=12.6, 2.1 Hz, 1H), 4.11 (t, J=9.2 Hz, 1H), 3.97-3.88 (m, 2H), 3.61 (t, J=9.0 Hz, 1H), 3.49 (d, J=9.4 Hz, 1H), 2.94 (s, 3H), 1.30-0.90 (m, 28H). ¹³C NMR (126 MHz, methanol-d₄): δ 159.5, 154.8, 153.5, 138.1, 117.4, 85.2, 80.8, 78.3, 73.36, 70.4, 62.1, 28.2, 18.0, 17.8, 17.8, 17.7, 17.6, 14.9, 14.5, 13.9, 13.8.

### Example 23. Step 2. Compound BC-41

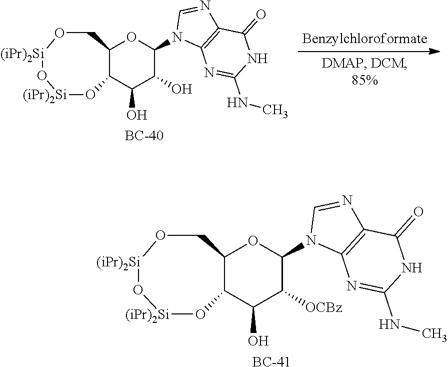

Benzylchloroformate (75 μL, 0.526 mmol, 4.28 equiv.) and DMAP (52.5 mg, 0.430 mmol, 3.41 equiv.) were added portion wise to a solution of BC-40 (72 mg, 0.126 mmol, 1.00 equiv.) in 4 mL DCM at 0° C. over a 45 min period. The resulting solution was stirred up to room temp. and stirred at that temp. for 15 min. The reaction mixture was then diluted with DCM and quenched with the addition of sat. aq. NaHCO₃. The organic layer was collected and additional organics were extracted 3× with DCM. The combined organics were dried using Na₂SO₄, filtered, and concentrated in vacuo. Flash column chromatography on silica using a gradient of 3-30% MeOH in DCM afforded BC-41 (73 mg, 85%) as a white solid. ¹H NMR (600 MHz, methanol-d₄): δ 7.81 (s, 1H), 7.30-7.23 (m, 3H), 7.11-7.05 (m, 2H), 5.65 (d, J=9.4 Hz, 1H), 5.22 (br m, 1H), 5.05 (d, J=12.3 Hz, 1H), 4.91 (d, J=12.3 Hz, 1H), 4.18 (dd, J=12.8, 2.3 Hz, 1H), 4.01 (t, J=9.3 Hz, 1H), 3.95 (d, J=11.4 Hz, 1H), 3.86 (t, J=9.1 Hz, 1H), 3.57 (dt, J=9.5, 1.4 Hz, 1H), 2.89 (s, 3H), 1.24-1.00 (m, 28H). ¹³C NMR (126 MHz, methanol-d₄): δ 159.3, 155.7, 154.8, 153.1, 137.8, 136.7, 129.6, 129.5, 128.8, 117.1, 80.9, 78.6, 75.7, 70.7, 70.4, 62.0, 28.2, 18.0, 17.8, 17.7, 17.5, 14.8, 14.5, 14.0, 13.8.

### Example 23. Step 3. Compound BC-42

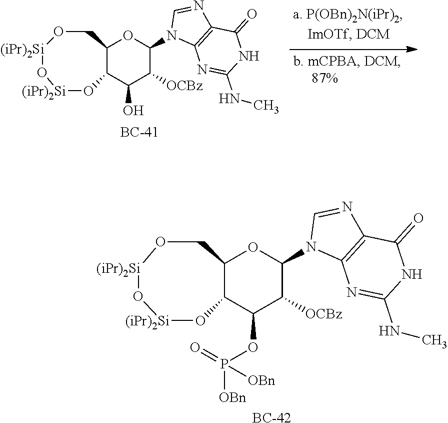

To an inhomogeneous solution of BC-41 (73 mg, 0.104 mmol, 1.00 equiv.) in DCM (3 mL) was added dibenzyl N,N-diisopropylphosphoramidite (70 μL, 0.208 mmol, 2.00 equiv.), and ImOTf (45 mg, 0.208 mmol, 2.00 equiv.). After 1 hr, an additional portion of dibenzyl N,N-diisopropylphosphoramidite (27.5 μL, 0.082 mmol, 0.78 equiv.), and ImOTf (11 mg, 0.050 mmol, 2.48 equiv.) were added and stirred for another hr. The resulting solution was cooled to 78° C. after which mCPBA (77% max, 95 mg, 0.425 mmol, 4.09 equiv.) was added. The resulting mixture was stirred up to 0° C. over a 1 hr period and was then diluted in DCM and quenched with the addition of sat. aq. NaHCO₃ (10 mL). The organic layer was collected and the aqueous layer was extracted 3× with DCM (15 mL each). Combined organics were dried with Na₂SO₄, filtered, and concentrated in vacuo. Flash column chromatography on silica using a gradient of 3-30% MeOH in DCM was performed, affording BC-42 (88 mg, 87%) as a white solid. ¹H NMR (600 MHz, methanol-d₄): δ 7.86 (s, 1H), 7.38-7.30 (m, 1OH), 7.24-7.19 (m, 3H), 7.02-6.97 (m, 2H), 5.75 (d, J=8.9 Hz, 1H), 5.72-5.60 (br m, 1H), 5.02-4.95 (m, 4H), 4.94 (d, J=12.2 Hz, 1H), 4.76 (q, J=8.8 Hz, 1H), 4.66 (d, J=12.3 Hz, 1H), 4.24 (t, J=9.2 Hz, 1H), 4.17 (dd, J=12.9, 2.1 Hz, 1H), 3.98 (dd, J=12.9, 1.6 Hz, 1H), 3.64 (dt, J=9.5, 1.9 Hz, 1H), 2.90 (s, 3H), 1.16-0.87 (m, 28H). ¹³C NMR (126 MHz, methanol-d₄): 159.3, 155.2, 154.8, 153.0, 138.0, 137.1 (d, J=6.7 Hz), 136.9 (d, J=6.5 Hz), 136.5, 129.8, 129.7, 129.6, 129.5, 129.4, 129.3, 128.8, 82.2, 82.1, 80.1, 76.3, 71.2 (d, J=5.8 Hz), 71.0 (d, J=5.7 Hz), 70.9, 69.9 (d, J=5.0 Hz), 61.8, 28.2, 18.0, 17.9, 17.8, 17.7, 17.5, 14.4, 14.3, 14.2, 14.1. HRMS (ESI) m/z: [M+H]⁺ calcd for C₄₆H₆₃N₅O₁₂PSi₂⁺ 964.3744; found 964.3733.

### Example 23. Step 4. Compound BC-43

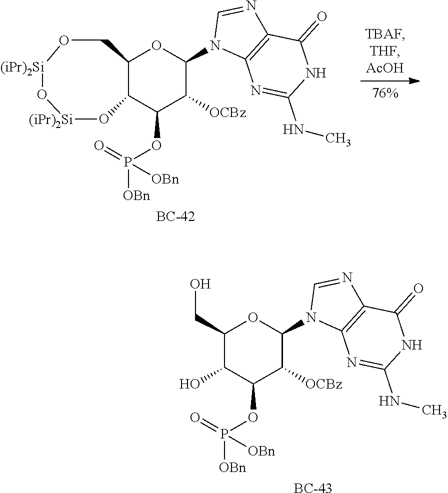

To a solution of BC-42 (88 mg, 0.091 mmol, 1.00 equiv.) in THF (3.5 mL) containing AcOH (15 uL, 0.26 mmol) at 0° C. was added TBAF (1M in THF, 270 μL, 0.270 mmol, 2.97 equiv.). After stirring for 5 hr cold (cooling with an ice-water bath), additional TBAF (50 μL, 0.050 mmol, 0.55 equiv) and AcOH (5 μL) was added, and the resulting solution stirred for an additional 2 hr up to room temp. until majority of starting material was consumed. An additional portion of AcOH was added (100 μL) and the solution was then concentrated to remove THF. Flash column chromatography on silica using a gradient of 5-40% MeOH in DCM was performed, affording BC-43 (48 mg, 76%). Samples contained residual TBAF fragments (10% w/w) which were unable to completely separate with subsequent purification. ¹H NMR (600 MHz, methanol-d₄): δ 7.97 (s, 1H), 7.42-7.26 (m, 10H), 7.25-7.17 (m, 3H), 7.00-6.94 (m, 2H), 5.78 (d, J=9.2 Hz, 1H), 5.55-5.44 (br m, 1H), 5.11 (d, J=7.6 Hz, 1H), 5.10 (d, J=7.9 Hz, 1H), 5.00 (d, J=7.6 Hz, 2H), 4.86 (d, J=12.3 Hz, 1H), 4.75 (q, J=9.0 Hz, 1H), 4.58 (d, J=12.3 Hz, 1H), 3.95-3.88 (m, 2H), 3.80 (dd, J=12.4, 5.1 Hz, 1H), 3.69 (ddd, J=9.8, 5.1, 2.1 Hz, 1H), 2.89 (s, 3H). ¹³C NMR (126 MHz, methanol-d₄): δ 159.3, 155.2, 154.9 153.0, 137.8, 137.2 (d, J=7.3 Hz), 137.1 (d, J=7.3 Hz), 136.3, 129.6, 129.5, 129.1, 128.9, 128.8, 116.9, 82.7 (d, J=6.8 Hz), 80.7, 76.8, 71.1 (d, J=6.0 Hz), 70.9, 69.7 (d, J=3.7 Hz), 61.9, 28.2.

### Example 23. Step 5. Compound BC-44

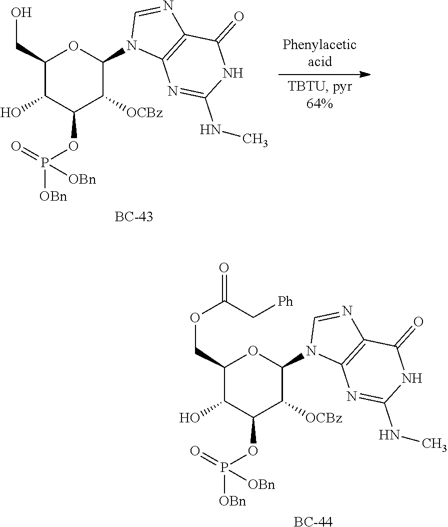

Phenylacetic acid (15 mg, 0.110 mmol, 4.78 equiv.) and TBTU (32 mg, 0.100 mmol, 4.35 equiv.) were added to a solution containing BC-43 (16.5 mg, 0.023 mmol, 1.00 equiv.) and 1 mL dry pyridine. The resulting mixture was stirred for 6 hr at room temp., DCM (2 mL) and MeOH (0.5 mL) were added, the solution was transferred to a round-bottom flask, and then concentrated to dryness in vacuo ensuring all pyridine was removed. Flash column chromatography on silica using a gradient of 2.5-30% MeOH in DCM was performed, affording BC-44 (12 mg, 64%) as a colorless solid. Samples contained some residual phenylacetic acid. ¹H NMR (600 MHz, methanol-d₄): δ 7.70 (s, 1H), 7.39-6.91 (m, 25H), 5.73 (d, J=9.2 Hz, 1H), 5.44-5.29 (br m, 1H), 5.10 (dd, J=7.9, 3.6 Hz, 2H), 4.99 (d, J=7.6 Hz, 2H), 4.85 (m, 1H), 4.73 (q, J=8.9 Hz, 1H), 4.58 (d, J=12.3 Hz, 1H), 4.53 (dd, J=12.3, 2.1 Hz, 1H), 4.33 (dd, J=12.2, 5.3 Hz, 1H), 3.91-3.86 (m, 1H), 3.84 (t, J=9.4 Hz, 1H), 3.68 (d, J=14.9 Hz, 1H), 3.64 (d, J=14.9 Hz, 1H), 2.89 (s, 3H).

### Example 23. Step 6. mgglu #31 (BC-45)

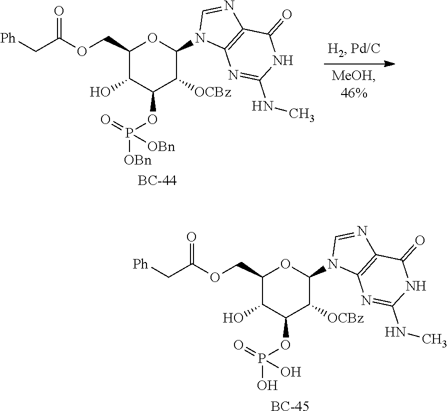

A suspension containing BC-44 (12 mg, 0.014 mmol, 1.00 equiv.), Pd/C (18 mg, 10% w/w), formic acid (200 μL) and MeOH (4 mL) was sparged with Ar for 5 min. then switched to H₂, through which the suspension was rapidly stirred under for 2 hr. After sparging with Ar, the reaction mixture was filtered through celite and washed with MeOH/H₂O and the collected filtrate was concentrated almost to dryness in vacuo and the resulting solution was loaded on celite. Flash column chromatography on C18 using a gradient of 1-100% ACN in H₂O (w/ 0.1% formic acid) afforded mgglu #31 (BC-45, 3.5 mg, 46%). mgglu #31 was found as an isomeric peak on C18 in C. briggsae endo-metabolome samples form/z=526.1333 by HPLC-HRMS (see Figures S3c and S11 for co-elution and MS² data, respectively). Chromatographic Method B was used.

### Example 24. Syntheses of Additional Compounds

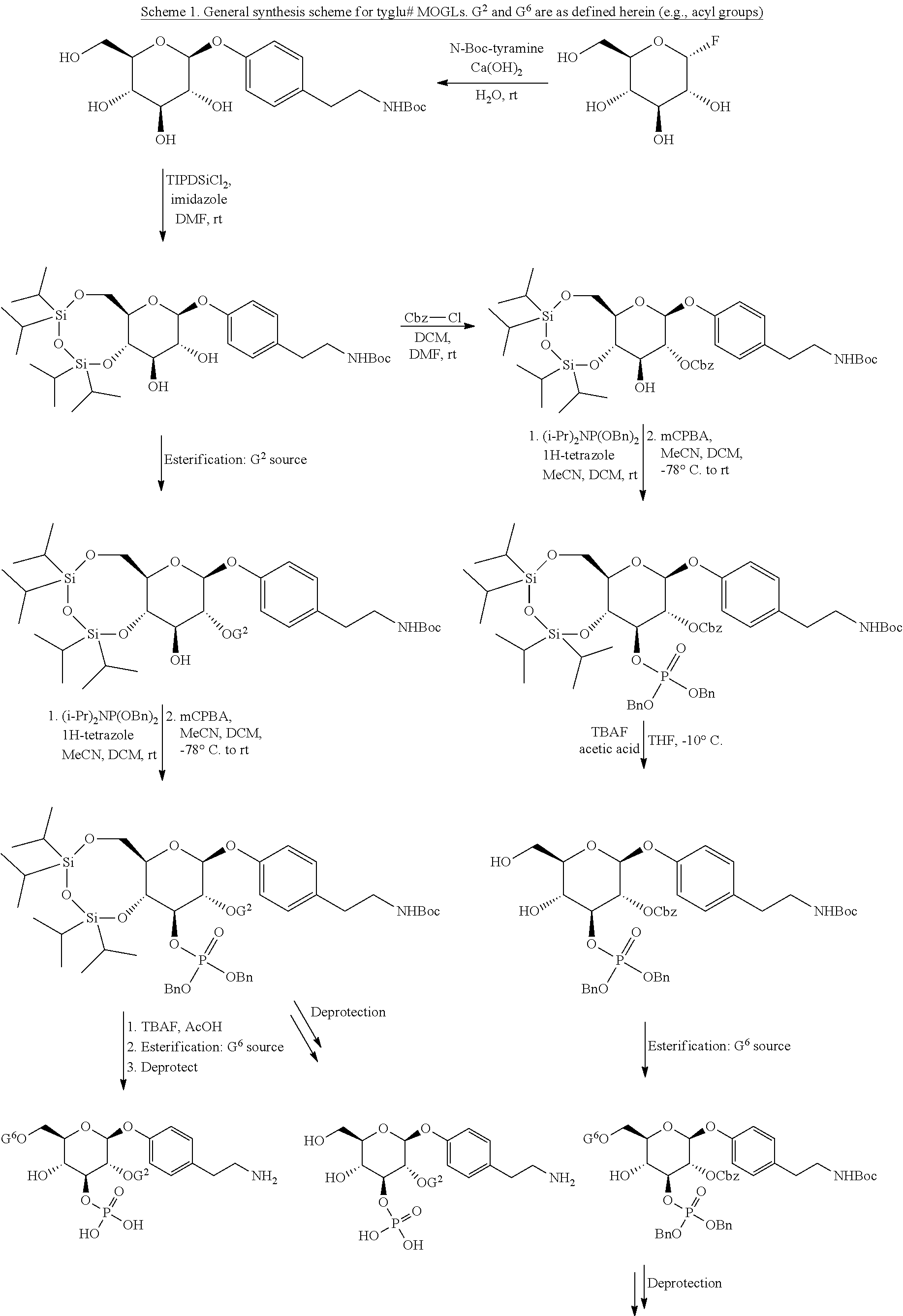

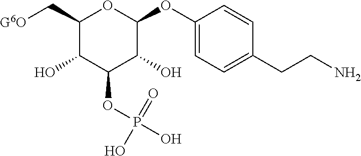

Tyglu synthesis is achieved by coupling N-Boc-tyramine with α-D-fluoroglucose, for selective preparation of the O-linked tyramine-glucoside, as reported for the sngl syntheses, and followed by 4,6-di-O-protection using 1,3-dichloro-1,1,3,3-tetraisopropyldisiloxane. Wadzinski, et. al., Nature Chemistry 10, 644-652 (2018); Yu, J. et al. “Parallel pathways for serotonin biosynthesis and metabolism in C. elegans.” Nat Chem Biol (Accepted). 2-O-acylated tyglu derivatives is prepared via esterification (e.g. using EDC/DMAP) with different carboxylic acids, which results in preferential acylation of the 2-position, followed by 3-O-phosphorylation (e.g. as described for the syntheses of sngl #4) and subsequently deprotection (e.g. using tetrabutylammonium fluoride) to furnish the target tyglu MOGLs. 6-O-acylated tyglu is synthesized by first protecting the 2-OH in the 4,6-diprotected intermediate above with benzyl chloroformate, then 3-O-phosphate is installed using similar procedure as above. Next 6-O-esterification is achieved using esterification on the 4,6-deprotected precursor, followed by subsequent deprotections steps to furnish the target tyglu compounds.

Oglu synthesis is achieved by coupling the phenolic OH of N—O-di-Boc protected octopamine (tert-butyl (S)-(2-((tert-butoxycarbonyl)oxy)-2-(4-hydroxyphenyl)ethyl)carbamate) with α-D-fluoroglucose as above. 2-O-acylated and 6-O-acylated oglu can be produced using procedures analogous to those outlined above for the synthesis of tyglu MOGLs.

The synthesis of angl #7 is achieved by coupling the unprotected precursor angl #1 and 2-methylbutanoic acid. The synthesis of angl #8 is achieved using a procedure analogous to that outlined above for 6-O-acylated tyglu MOGLs.

To selectively synthesize angl #6, N-Boc-anthranilic acid is coupled with glucose, then the protected product is 4,6-di-O-protected using 1,3-dichloro-1,1,3,3-tetraisopropyldisiloxane. 2-OH is protected with benzyl chloroformate, followed by installation of methoxybenzyl-protected 3-O-phosphate. A deprotection/esterification sequence analogous to the above examples is performed to achieve the final product angl #6.

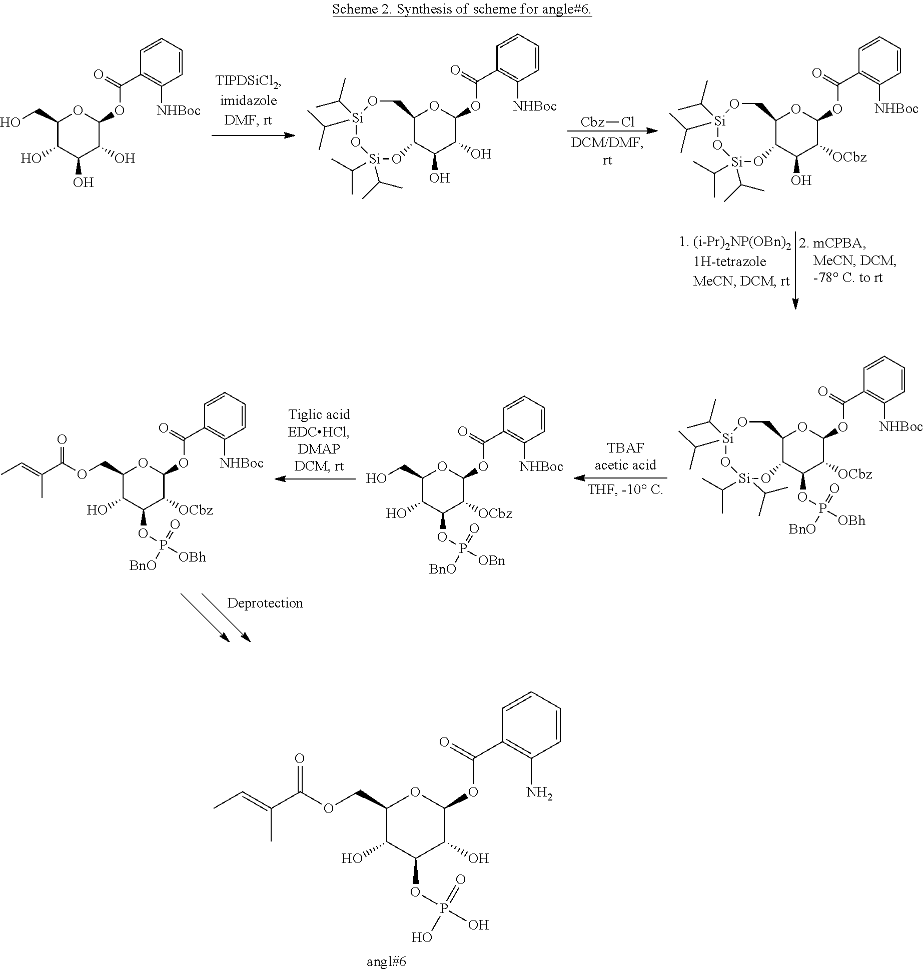

The following compounds have also been synthesized in a manner similar to the ones shown in the Examples above, confirmed by NMR and mass spectrometry. The structures of each are shown in Table S5.

Exemplary synthetic compounds confirmed by HRMS.

| Compound | HRMS (ESI) m/z calcd for |
|---|---|
| angl#5 | C₁₈H₂₃NO₈ [M + H]⁺ 382.1496, found 382.1479. |
| oglu#1 | C₄₂H₂₂NO₇ [M + H]⁺ 316.13908, found 316.13846. |
| oglu#2 | C₁₂H₂₃NO₁₀P [M + H]⁺ 396.10541, found 396.10501. |
| sngl#1 | C₁₈H₂₄N₂O₇ [M + Na]⁺ 403.1476, found 403.1485. |
| sngl#3 | C₂₅H₂₉N₃O₈ [M + H]⁺ 500.2027, found 500.2005. |

### REFERENCES FOR PRECEEDING SECTIONS A (“MATERIALS AND METHODS”) AND B (“SYNTHETIC PROCEDURES”)

(1) Eisenmann, D. M. WormBook. WormBook 2005.
(2) Le, H. H.; Wrobel, C. J. J.; Cohen, S. M.; Yu, J.; Park, H.; Helf, M. J.; Curtis, B. J.; Kruempel, J. C.; Rodrigues, P. R.; Hu, P. J.; Sternberg, P. W.; Schroeder, F. C. Modular Metabolite Assembly in Caenorhabditis Elegans Depends on Carboxylesterases and Formation of Lysosome-Related Organelles. Elife 2020, 9, 1-42. doi.org/10.7554/eLife.61886.
(3) Dric Notredame, C. Â.; Higgins, D. G.; Heringa, J. T-Coffee: A Novel Method for Fast and Accurate Multiple Sequence Alignment. doi.org/10.1006/jmbi.2000.4042.
(4) Altschul, S. F.; Wootton, J. C.; Gertz, E. M.; Agarwala, R.; Morgulis, A.; Schaffer, A. A.; Yu, Y. K. Protein Database Searches Using Compositionally Adjusted Substitution Matrices. FEBS Journal. 2005. doi.org/10.1111/j.1742-4658.2005.04945.x.
(5) Edgar, R. C. MUSCLE: Multiple Sequence Alignment with High Accuracy and High Throughput. Nucleic Acids Res. 2004. doi.org/10.1093/nar/gkh340.
(6) Saitou, N.; Nei, M. The Neighbor-Joining Method: A New Method for Reconstructing Phylogenetic Trees. Mol. Biol. Evol. 1987, 4 (4), 406-425. doi.org/10.1093/oxfordjournals.molbev.a040454.
(7) Felsenstein, J. CONFIDENCE LIMITS ON PHYLOGENIES: AN APPROACH USING THE BOOTSTRAP. Evolution (N. Y). 1985, 39 (4), 783-791. doi.org/10.1111/j.1558-5646.1985.tb00420.x.
(8) Jones, D. T.; Taylor, W. R.; Thornton, J. M. The Rapid Generation of Mutation Data Matrices from Protein Sequences. Bioinformatics 1992. doi.org/10.1093/bioinformatics/8.3.275.
(9) Kumar, S.; Stecher, G.; Li, M.; Knyaz, C.; Tamura, K. MEGA X: Molecular Evolutionary Genetics Analysis across Computing Platforms. Mol. Biol. Evol. 2018, 35 (6), 1547-1549. doi.org/10.1093/molbev/msy096.
(10) Kumar, S.; Stecher, G.; Tamura, K. MEGA7: Molecular Evolutionary Genetics Analysis Version 7.0 for Bigger Datasets. Mol. Biol. Evol. 2016. doi.org/10.1093/molbev/msw054.
(11) Cohen, S.; Sternberg, P. Genome Editing of Caenorhabditis Briggsae Using CRISPR/Cas9 Co-Conversion Marker Dpy-10. microPublication Biol. 2019, 000171. doi.org/10.17912/micropub.biology.000171.
(12) Wang, H.; Park, H.; Liu, J.; Sternberg, P. W. An Efficient Genome Editing Strategy To Generate Putative Null Mutants in Caenorhabditis Elegans Using CRISPR/Cas9. G3&amp; #58; Genes|Genomes|Genetics 2018, 8 (11), 3607-3616. doi.org/10.1534/g3.118.200662.
(13) O'Donnell, M. P.; Fox, B. W.; Chao, P. H.; Schroeder, F. C.; Sengupta, P. A Neurotransmitter Produced by Gut Bacteria Modulates Host Sensory Behaviour. Nature 2020, 583 (7816), 415-420. doi.org/10.1038/s41586-020-2395-5.
(14) Smith, C. A.; Want, E. J.; O'maille, G.; Abagyan, R.; Siuzdak, G. XCMS: Processing Mass Spectrometry Data for Metabolite Profiling Using Nonlinear Peak Alignment, Matching, and Identification. 2006, 78 (3), 779-787. doi.org/10.1021/ac051437y.
(15) Tautenhahn, R.; Bottcher, C.; Neumann, S. Highly Sensitive Feature Detection for High Resolution LC/MS. BMC Bioinformatics 2008, 9, 1-16. doi.org/10.1186/1471-2105-9-504.
(16) Ludewig, A. H.; Artyukhin, A. B.; Aprison, E. Z.; Rodrigues, P. R.; Pulido, D. C.; Burkhardt, R.
N.; Panda, O.; Zhang, Y. K.; Gudibanda, P.; Ruvinsky, I.; Schroeder, F. C. An Excreted Small Molecule Promotes C. Elegans Reproductive Development and Aging. Nat. Chem. Biol. 2019, 15 (8), 838-845. doi.org/10.1038/s41589-019-0321-7.
(17) Messaoudi, S.; Sancelme, M.; Polard-Housset, V.; Aboab, B.; Moreau, P.; Prudhomme, M.
   Synthesis and Biological Evaluation of Oxindoles and Benzimidazolinones Derivatives. Eur. J. Med. Chem. 2004, 39 (5), 453-458. doi.org/10.1016/j.ejmech.2004.01.001.
(18) Reuss, S. H. Von; Bose, N.; Srinivasan, J.; Yim, J. J.; Judkins, J. C.; Steinberg, P. W.; Schroeder, F. C. Comparative Metabolomics Reveals Biogenesis of Ascarosides, a Modular Library of Small-Molecule Signals in C. Elegans. 2012.

### Example 25

Interaction of MOGLs with the Proteasome

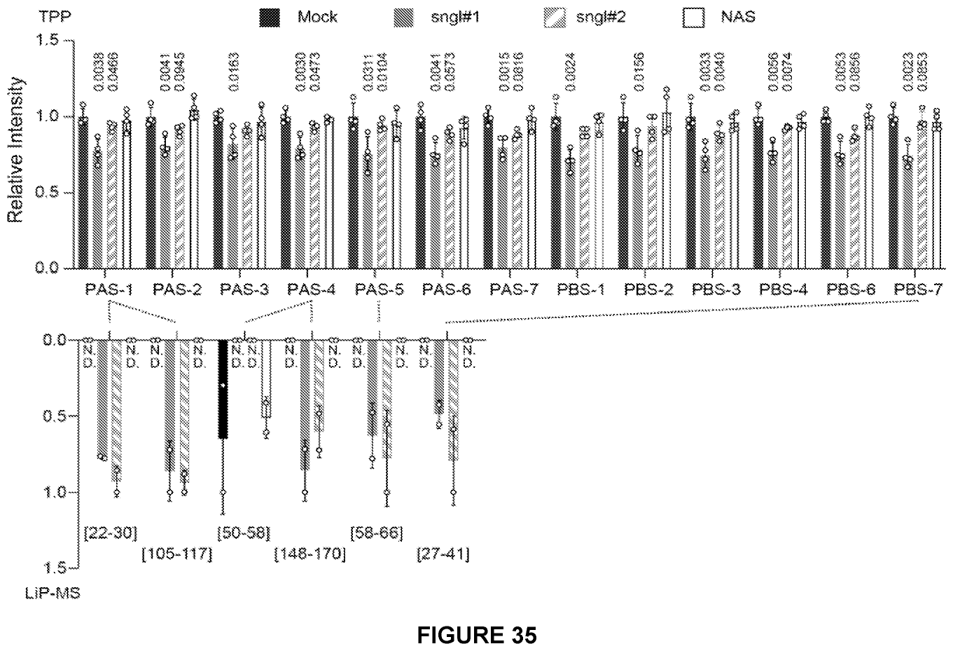

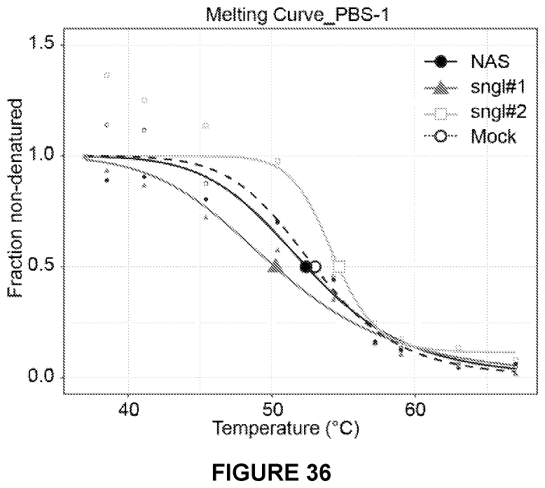

To investigate the cellular interactors of MOGLs, two independent approaches were applied: thermal proteome profiling (TPP) and limited proteolysis-coupled mass spectrometry (LiP-MS), to uncover binding events upon compound treatment of C. elegans lysates using MOGLs of the sngl-class as an example. Overlap of positive hits in the two assays revealed that thermal stability (as measured by TPP) and tendency toward proteolysis (as measured by LiP-MS) of proteasome alpha and beta subunits were changed upon incubation with the MOGLs sngl #1 and sngl #2 (FIG. 35). When heating the proteome-compound mixture at an optimal melting temperature for C. elegans proteins, sngl #1 and sngl #2 (phosphorylated sngl #1), but not N-acetylserotonin (NAS) (used as a control compound), significantly change the thermal stability of majority detected proteasome alpha and beta subunits (FIG. 36). Moreover, there were multiple peptides from proteasome alpha subunits and one peptide from the beta unit that were differential, with absence-present contrast, in the LiP-MS experiment (FIG. 35). Results from these two assays which reflect distinct biophysical properties of protein-small molecule binding, strongly support interaction between sngl #1 and sngl #2 with the proteasome.

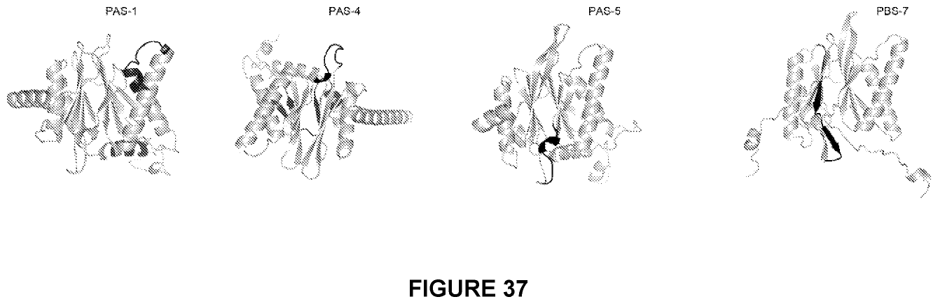

To obtain mechanistic insight into the binding of proteasome with sngl #1/sngl #2, LiP-MS data were analyzed to reveal peptides around sub-structures of proteins where the binding occurs. We used AlphaFold to predict structures and found that the differential peptides generally resided in or close to the two center layers of beta sheets in a single proteasome subunit, which is likely to affect proper folding of these proteins (FIG. 37).

### Methods

C. elegans lysate preparation and compound incubation. Synchronized pah-1(syb3596); tph-1(mg280) double knockout C. elegans were grown with ΔtnaA E. coli (JW3686-7) for two generations; growth conditions and harvesting procedures were as described in the ‘Nematode cultures’ section above. Worms were lysed by sonication with lysis buffer (25 mM Tris pH 7.5, 150 mM NaCl, 1.5 mM MgCl₂.1 mM phenylmethylsulfonyl fluoride, 1× protease inhibitor cocktail (Roche), 1 mM sodium fluoride) for 5 min (3 s on/off pulse cycle at Amp 100). Protein concentration was determined by Bradford assay (MilliporeSigma) and diluted to 1 mg/mL by lysis buffer. To each aliquot of lysate was added compound (sngl #1, sngl #2, N-acetylserotonin, and solvent (ethanol) control (Mock)) to a final concentration of 0.1 mM and the mixtures were shaken for 15 min at room temperature.

Thermal proteome profiling (TPP) sample preparation. Methods were modified from published protocol (Franken, H., T. et al. Nature Protocols 10(10): 1567-1593). Lysate-compound mixture were aliquoted into 0.2-mL PCR tubes, heated at specified temperatures for 3 min using a Thermal Cycler (BioRad), centrifuged at 16,000 g at 4° C. for 5 min, and the supernatant were transferred into Eppendorf tubes. Four volumes of prechilled (−20° C.) acetone were added to each sample for precipitation of proteins overnight. Samples were centrifuged at 16,000 g at 4° C. for 20 min, washed once by methanol, and the resulting protein pellets were dissolved in urea buffer (50 mM ammonium bicarbonate, 2 M thiourea, 6 M urea). To digest proteins for mass spectrometry analysis, proteins were first reduced by dithiothreitol (final concentration 5 mM) for 30 min at room temperature, alkylated by iodoacetamide (final concentration 15 mM) for 30 min at room temperature in the dark, digested by LysC/Trypsin Mix (Promega) (enzyme/substrate=1/30, w/w) based on manufacturer's instructions. Samples were acidified with trifluoroacetic acid to pH ˜3, and desalted in a C18 96-well Plate (Sep-Pak, Waters), dried by a Speedvac. Peptides were measured by nanoHPLC-MS/MS by standard methods.

Limited proteolysis-coupled mass spectrometry (LiP-MS) sample preparation. Methods were modified from published protocol (Schopper, S., A. et al. Nature Protocols 12(11): 2391-2410). Lysate-compound mixture were aliquoted into 0.2-mL PCR tubes, proteinase K (enzyme/substrate=1/100, w/w) was added, incubated at 25° C. for 5 min in a Thermal Cycler (BioRad), heated at 98° C. for 5 min for irreversible denaturation of proteinase K, and stored in −20° C. overnight. Procedures for protein digestion and peptide desalting for mass spectrometry analysis were the same as described in ‘Thermal proteome profiling (TPP) sample preparation’ section above.

### Example 26

### MOGLs Increase Lifespan and are Required for Stress Response

As described herein, MOGL biosynthesis is strongly upregulated during starvation. When testing whether MOGL production via CEST-1.2 is required for starvation survival, the results show that lifespan of starved cest-1.2 mutant adults was significantly reduced compared to wildtype. (FIG. 33c). For the purpose of the survival experiment (right panel), both wild-type and cest-1.2 mutant animals were grown under well fed conditions, using E. coli OP50 bacteria, until they reached adulthood (adult day 1). Then the worms were transferred to media without food and their survival was scored every 3-10 hours, until all animals were dead. For the bagging assay (left panel), the same experimental procedure was used, except that bagging (egg hatching inside the parent) was scored.

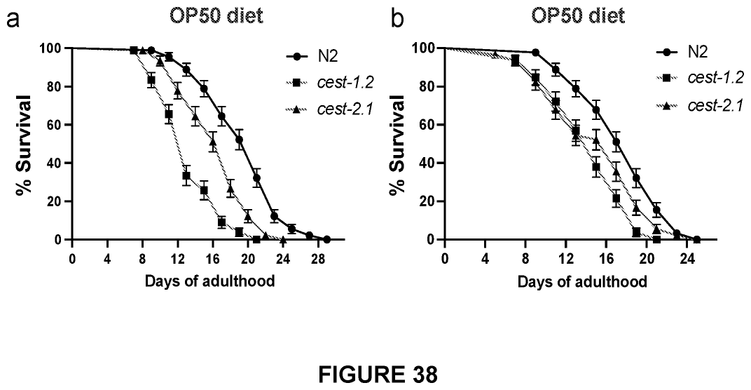

This experiment found that MOGL production via CEST-1.2 is required for normal lifespan, which suggest that MOGL production protects regulates kinase-dependent stress response pathways, such as oxidative stress, thermal stress, e.g. via binding to kinases and other components of these signaling pathways (FIG. 38). Lifespan assays were performed on agar plates using OP50 as food at 20° C., using standard procedures (see www.WormBook.org). Animals were scored as dead or alive every 2 days and scored as dead if they failed to respond to touch.

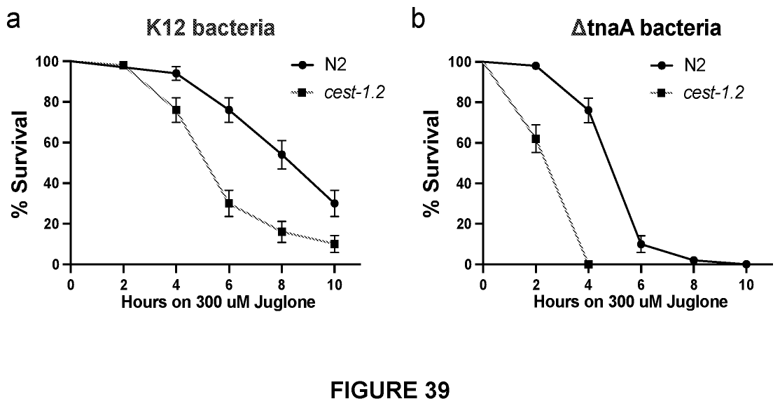

We further found that mutants lacking MOGL production via CEST-1.2 or CEST-2.1 are more sensitive to the oxidant juglone (tested at 300 μM) compared to wildtype, suggesting that the modular glucosides are protective by modulating the oxidative stress response. When one family of MOGLs, the indole-containing glucosides (iglu's), were tested it was found that C. elegans fed a diet of E. coli that is unable to produce indole (ΔtnaA)—and thus is unable to produce iglu-style MOGLs—is indeed more sensitive to 300 μM juglone exposure, suggesting that iglu-type MOGLs modulate the oxidative stress response in C. elegans. For the purpose of this experiment, all animals were grown on normal media seeded with either E. coli K12 or ΔtnaA bacteria until reaching adulthood (adult day 1). The assay was performed on freshly made media supplemented with 300 μM juglone (Sigma #H47003) and seeded with E. coli OP50 bacteria. Animals were scored as dead or alive every 2 hours, for a total of 10 hours. Death was measured as a failure to respond to respond to a gentle touch. See FIG. 39.

### Example 27

### Activity of MOGLs in Broad-Based In-Vitro Assays Using Human Cell Lines

To evaluate the effects of MOGLs in primary human tissues and to demonstrate the relative activities of the MOGLs described herein for the treatment of specific human diseases, pure samples of each compound in Table S5 are synthesized according to the procedures described herein and evaluated using the BioMAP® Phenotypic Profiling Assay system. See for example: Kim, et. al., Cell Chemical Biology, 27:6, 698-707 (2020).

MOGLs are screened in vitro against using a panel of 12 human primary cell-based co-culture systems (venular endothelial cells, lung fibroblasts, and peripheral blood mononuclear cells, PBMCs) that model various tissues and diseases. Protein biomarker readouts in these mixed cell systems are used to quantify the effects of the MOGLs.

Screening is conducted with the BioMAP® Diversity PLUS assay performed by DiscoverX. Human primary cells in BioMAP systems are used at early passage (passage 4 or earlier) to minimize adaptation to cell culture conditions and preserve physiological signaling responses. All cells are from a pool of multiple donors (n=2-6), commercially purchased and handled according to the recommendations of the manufacturers. Human blood derived CD14+ monocytes are differentiated into macrophages in vitro before being added to the/Mphg system. Abbreviations are used as follows: Human umbilical vein endothelial cells (HUVEC), Peripheral blood mononuclear cells (PBMC), Human neonatal dermal fibroblasts (HDFn), B cell receptor (BCR), T cell receptor (TCR) and Toll-like receptor (TLR). Cell ty pes and stimuli used in each system are as follows: 3C system [HUVEC+(IL-1β, TNFα and IFNγ)], 4H system [HUVEC+(IL-4 and histamine)], LPS system [PBMC and HUVEC+LPS (TLR4 ligand)], SAg system [PBMC and HUVEC+TCR ligands (1×)], BT system [CD19+ B cells and PBMC+(α-IgM and TCR ligands (0.001×)], BF4T system [bronchial epithelial cells and HDFn+(TNFα and IL-4)], BE3C system [bronchial epithelial cells +(IL-1β, TNFα and IFNγ)], CASM3C system [coronary artery smooth muscle cells +(IL-1β, TNFα and IFNγ)], HDF3CGF system [HDFn+(IL-1β, TNF∘, IFNγ, EGF, bFGF and PDGF-BB)], KF3CT system [keratinocytes and HDFn+(IL-1β, TNFα and IFNγ)], MyoF system [differentiated lung myofibroblasts+(TNFα and TGFβ)] and /Mphg system [HUVEC and M1 macrophages+Zymosan (TLR2 iigand)]. Systems are derived from either single cell types or co-culture systems. Adherent cell types are cultured in 96- or 384-well plates until confluence, followed by the addition of PBMC (SAg and LPS systems). The BT system consists of CD19+B cells co-cultured with PBMC and stimulated with a BCR activator and low levels of TCR stimulation. MOGLs are prepared in DMSO (final concentration ≤0.1%) and added at a final concentration of 21 μM, 1 h before stimulation and remain in culture for 24 h (48 h: MyoF system; 72 h: BT system (soluble readouts); 168 h: BT system (secreted IgG)). Each plate contains drug controls, negative controls (e.g., non-stimulated conditions) and vehicle controls (e.g., 0.1% DMSO) appropriate for each system. Direct ELISA is used to measure biomarker levels of cell-associated and cell membrane targets. Soluble factors from supernatants are quantified using either HTRF@R detection, bead-based multiplex irnmunoassay or capture ELISA. Overt adverse effects of test agents on cell proliferation and viability (cytotoxicity) are detected by sulforhodamine B (SRB) staining, for adherent cells, and alamarBlue® reduction for cells in suspension. For proliferation assays, individual cell types are cultured at sub-confluence and measured at time points optimized for each system (48 h: 3C and CASM3C systems; 72 h: BT and HDF3CGF systems; 96 h: SAg system). Cytotoxicity for adherent cells is measured by SRB (24 h: 3C, 4H, LPS, SAg, BF4T, BE3C, CASM3C, HDF3CGF, KF3CT, /Mphg systems; 48 h: MyoF sy stem), and by alamarBlue staining for cells in suspension (24 h: SAg system; 42 h: BT system) at the time points indicated.

Results from the MOGL screening assays described above are analyzed as follows: Biomarker measurements in a MOGL-treated sample are divided by the average of control samples (at least 6 vehicle controls from the same plate) to generate a ratio that is then log 10 transformed. Significance prediction envelopes are calculated using historical vehicle control data at a 95% confidence interval. The results are further interpreted through Profile-, Benchmark-, Similarity- and Cluster Ananlyses as described below:

Profile Analysis. Bioactivities are confirmed when 2 or more consecutive MOGL concentrations change in the same direction relative to vehicle controls, are outside of the significance envelope, and have at least one concentration with an effect size >20% (log 10 ratiol >0.1). Biomarker key activities are described as modulated if these activities increase in some systems, but decrease in others. Cytotoxic conditions are noted when total protein levels decrease by more than 50% (log 10 ratio of SRB or alamarBlue levels <−0.3). A MOGL is considered to have broad cytotoxicity when cytotoxicity is detected in 3 or more systems. Concentrations of MOGLs with detectable broad cytotoxicity are excluded from biomarker activity annotation and downstream benchmarking, similarity search and cluster analysis. Antiproliferative effects of tested MOGLs are defined by an SRB or alamarBlue log 10 ratio value <−0.1 from cells plated at a lower density. Cytotoxicity and antiproliferative arrows only require one concentration to meet the indicated threshold for profile annotation.
Benchmark Analysis. Common biomarker readouts are noted when the readout for both profiles are outside of the significance envelope with an effect size >20% in the same direction. Differentiating biomarkers are annotated when one profile has a readout outside of the significance envelope with an effect size >20%, and the readout for the other profile is either inside the envelope or in the opposite direction.
Similarity Analysis. Common biomarker readouts are noted when the readout for both profiles is outside of the significance envelope with an effect size >20% in the same direction.
Concentrations of MOGLs that have 3 or more detectable systems with cytotoxicity are excluded from similarity analysis. Concentrations of MOGLs that have 1-2 systems with detectable cytotoxicity are included in the similarity search analysis, along with an overlay of the database match with the top concentration of the test agent.
Cluster Analysis. Cluster analysis (function similarity map) uses the results of pairwise correlation analysis to project the “proximity” of MOGL activity profiles from multi-dimensional space into two dimensions. Functional clustering of the MOGL profiles are generated during this analysis using Pearson correlation values for pairwise comparisons of the profiles for each agent at each concentration, and then subjects the pairwise correlation data to multidimensional scaling. MOGLs that do not cluster with one another are interpreted as mechanistically distinct.

### Example 28

### In Vitro Screening of MOGLs for Proteasome Modulatory Activity

To assess the activity of MOGLs in inhibiting proteasome activity, an assay is undertaken to measure the accumulation of undegradable undegradable polyubiquitinated proteins in by measuring the size and/or abundance of nuclear aggregations of ubiquitinated proteins termed “aggresomes”, using a cell- and imaging-based screening system adapted from a method reported in Marine Drugs, 2018 October; 16(10): 395. DOI:10.3390/mnd16100395.

Synthetic samples of each of the MOGLs in Table S5 are diluted at 10-fold intervals between 10 nM and 1 mM.

The controls and MOGLs are diluted and dispensed into culture plates at the concentrations noted above along with two known proteasome inhibitors as positive controls (Bortezomib, 13.1 nM and 7.76 nM; and MG132, 0.97 μM and 1.65 μM).

HEK293T cells transiently expressing EGFP-UL76 are seeded at 1×10⁶ cells onto 6-cm culture dishes one day before transfection. Then, 3 μg of plasmid DNA pEGFP-UL76 is transfected into HEK293T cells mediated by Lipofectanine Plus and Lipofectamine (Thermo Fisher Scientific, Waltham, MA, USA). After 3 h of transfection, the transfected cells are trypsinized and dispensed into black glass-bottom 96-well plates at 1×10⁴ cells per well in a volume of 200 μL per well, including the indicated compound at each concentration with three repeats. The culture plates containing the cells and tested compounds are incubated at 5% CO₂ and 37° C. for 48 h. Subsequently, the cells are fixed in 1% paraformaldehyde for 10 min and simultaneously permeabilized with 0.1% IGEPAL® CA-630, then stained with 1.5 μg/mL DAPI on ice for 30 min. After extensive washing with PBS, the cells are submerged in PBS, sealed in the dark, and stored at 4 CC.

Image acquisition is accomplished using an ImageXpress Micro Widefield HTCJS system (Molecular Device, San Jose, CA, USA) under an objective magnification of 20×Ph1. Each well is acquired in 25 consecutive images in 5×5 sites with 38% well area coverage. Two modules of MetaExpress, Cell Scoring and Multi-Wavelength Cell Scoring, are employed to analyze the high-content measurements. Cell Scoring is configured to define nuclei marked by 4′ 6-diamidino-2-phenylindole (DAPI) staining with diameters of 8 to 15 μm, whereas EGFP-UL76 aggresomes have diameters of 1 to 50 μm. The intensity of the above background was determined according to the manufacturer's instructions. Multi-Wavelength Cell Scoring was configured to classify aggresomes by size into pit and vesicle categories. The pit category contained aggresomes with diameters of 1 μm to 5 μm, whereas the vesicle category contained aggresomes with diameters of 5 μm to 50 μm. The data are compiled into cell-by-cell and site-by-site measurements. The relative ratios are calculated by normalization to the value of the control without MOGL treatment.

Ratios are calculated by comparison of the aggresome characteristics of MOGL treated cells to the control value obtained without MOGL treatment. 1 MOGL treatments showing statistically-relevant lose-dependent increases the number and/or size of aggresomes relative to the negative control are confirmed to have proteasome inhibitory activity.

### C. Additional Supporting Tables

TABLE S1
NMR spectroscopic data for iglu#121 (25). ¹H (600 MHz), dqfCOSY
(600 MHz), HSQC (600 MHz), and HMBC (800 MHz) data were
acquired in methanol-d₄ (br., broad).

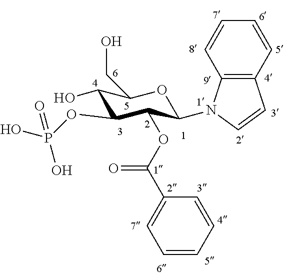

| | | δ ¹H [ppm] | |
|---|---|---|---|
| | δ ¹³C | (Multiplicity, | |
| Position | [ppm] | J_{HH}[Hz]) | HMBC |
| 1 | 84.4 | 5.92 (d, J_{1,2} = 9.2) | C-2, C-3, C-5, C-2′, C-9′ |
| 2 | 73.1 | 5.67 (t, J_{1,2} ≈ J_{2,3}) | C-1, C-3, C-1″ |
| 3 | 79.9 | 4.65 (br. m) | |
| 4 | 71.5 | 3.88 (br. m) | C-6 (weak) |
| 5 | 80.5 | 3.76 (br. m,) | C-6 (weak) |
| 6a | 62.3 | 3.80 (dd, J_{5,6a} = 5.7, | C-4 (weak), C-5 |
| | | J_{6a,6b} 11.6) | |
| 6b | | 3.95 (dd, J_{5,6b} = 1.6) | C-4, C-5(weak) |
| 2′ | 126.3 | 7.38 (d, J_{2′,3′} = 3.3) | C-1, C-3′, C-4′, C-5′, C-6′, |
| | | | C-8′, C-9′ |
| 3′ | 103.8 | 6.38 (d) | C-1(weak), C-2′, C-4′, C-5′, |
| | | | C-9′ |
| 4′ | 130.0 | | |
| 5′ | 121.2 | 7.41 (d, J_{5′,6′} = 7.8) | C-3′, C-4′, C-7′, C-8′(weak), |
| | | | C-9′ |
| 6′ | 120.7 | 6.95 (ddd, J_{6′,7′} = 7.4, | C-4′, C-5′(weak), C-7′, C-8′, |
| | | J_{6′,8′} = 0.7) | C-9′(weak) |
| 7′ | 122.5 | 7.07 (ddd, J_{7′,8′} = 8.3, | C-4′(weak), C-5′, C-6′(weak), |
| | | J_{5′,7′} = 1.3) | C-8′(weak), C-9′ |
| 8′ | 111.0 | 7.56 (d) | C-4′, C-6′ |
| 9′ | 137.5 | | |
| 1″ | 165.1 | | |
| 2″ | 126.2 | | |
| 3″ | 130.4 | 7.74 (dd, J_{3″,4″} = 8.2 | C-1″, C-4″ (weak), C-5″, C-7″ |
| | | J_{3″,5″} = 1.3) | |
| 4″ | 128.8 | 7.27 (dd, J_{4″,5″} = 7.8) | C-1″, C-3″, C-5″, C-6″ |
| 5″ | 133.6 | 7.43 (dd, J_{5′,6′} = 7.8) | C-3″, C-4″(weak), C- |
| | | | 6″(weak), C-7″ |
| 6″ | 128.8 | 7.27 (dd) | C-1″, C-4″, C-5″, C-7″ |
| 7″ | 130.4 | 7.74 (d) | C-1″, C-3″, C-5″, C-6″ (weak) |

TABLE S2
NMR spectroscopic data for iglu#401 (28). ¹H, dqfCOSY, HSQC and
HMBC data (all at 600 MHz) were acquired in methanol-d₄ (br., broad).

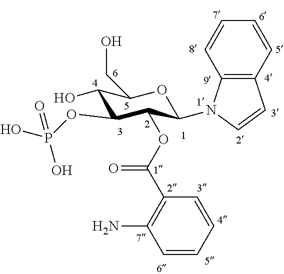

| | δ ¹³C | δ ¹H [ppm] | |
|---|---|---|---|
| Position | [ppm] | (J_{HH}[Hz]) | HMBC |
| 1 | 84.1 | 5.94 (d, J_{1,2} = 9.1) | C-2, C-5 (weak), C-2′, C-9′ |
| 2 | 72.0 | 5.75 (br. m) | C-1, C-3 |
| 3 | 81.4 | 4.72 (br. m) | |
| 4 | 70.8 | 3.89 (br. m) | |
| 5 | 80.1 | 3.78 (br. m) | |
| 6a | 62.1 | 3.81 (br. m, | |
| | | J_{6a,6b} = 11.5) | |
| 6b | | 3.95 (br. m) | |
| 2′ | 126.2 | 7.39 (d, J_{2′,3′} = 3.4) | C-3′, C-4′, C-9′ |
| 3′ | 103.7 | 6.40 (d) | C-2′, C-4′, C-9′ |
| 4′ | 130.0 | | |
| 5′ | 121.2 | 7.44 (d, J_{5′,6′} = 7.9) | C-3′, C-4′, C-7′, C-8′(weak), |
| | | | C-9′ |
| 6′ | 120.8 | 6.98 (dd, J_{6′,7′} = 7.4) | C-4′, C-7′(weak), C-8′, |
| | | | C-9′(weak), |
| 7′ | 122.6 | 7.08 (dd, J_{7′,8′} = 8.2) | C-5′, C-8′(weak), C-9′ |
| 8′ | 111.1 | 7.58 (d) | C-4′, C-6′ |
| 9′ | 137.4 | | |
| 1″ | 166.6 | | |
| 2″ | 112.2 | | |
| 3″ | 132.2 | 7.64 (d, J_{3″,4″} = 7.8) | C-1″, C-5″, C-7″ |
| 4″ | 117.4 | 6.51 (dd, J_{4″,5″} = 7.6) | C-2″, C-3″(weak), C-6″, |
| | | | C-5″(weak) |
| 5″ | 134.6 | 7.14 (dd, J_{5″,6″} = 8.3) | C-2″(weak), C-3″, |
| | | | C-6″(weak), C-7″ |
| 6″ | 118.0 | 6.64 (d) | C-1″, C-2″, C-4″ |
| 7″ | 149.4 | | |

TABLE S3
NMR spectroscopic data for iglu#101 (26). ¹H (800 MHz), dqfCOSY,
HSQC and HMBC data (all 800 MHz) were
acquired in methanol-d₄ (br., broad).

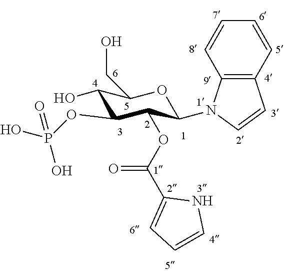

| | δ ¹³C | δ ¹H [ppm] | |
|---|---|---|---|
| Position | [ppm] | (J_{HH}[Hz]) | HMBC |
| 1 | 84.5 | 5.86 (d, J_{1,2} = 9.6) | C-2, C-3 (weak), C-2′, C-9′ |
| 2 | 71.9 | 5.64 (br. m) | |
| 3 | 81.6 | 4.69 (br. m) | |
| 4 | 71.0 | 3.90 (br. m) | |
| 5 | 80.3 | 3.76 (br. m) | |
| 6a | 62.2 | 3.80 (br. m) | |
| 6b | | 3.94 (br. m) | |
| 2′ | 126.2 | 7.37 (br. m) | C-1, C-3′, C-4′, C-5′, C-8′, C-9′ |
| 3′ | 103.9 | 6.39 (d, J_{2′,3′} = 3.1) | C-2′, C-4′, C-5′(weak), C-9′ |
| 4′ | 130.1 | | |
| 5′ | 121.3 | 7.43 (d, J_{5′,6′} = 7.7) | C-1 (weak), C-3′, C-4′, C-6′, |
| | | | C-7′, C-8′, C-9′ |
| 6′ | 120.9 | 6.98 (ddd, J_{6′,7′} = 7.5, | C-3′, C-4′, C-5′, C-7′, C-8′, |
| | | J_{6′,8′} = 0.8) | C-9′ |
| 7′ | 122.7 | 7.10 (ddd, J_{7′,8′} = 8.3) | C-3′ (weak), C-4′, C-5′, C-8′, |
| | | | C-9′ |
| 8′ | 111.1 | 7.56 (d) | C-4′, C-6′, C-9′ |
| 9′ | 137.4 | | |
| 1″ | 160.4 | | |
| 2″ | 122.2 | | |
| 4″ | 124.4 | 6.82 (d, J_{4″,5″} = 1.9) | C-1″, C-2″, C-5″, C-6″ |
| 5″ | 117.2 | 6.67 (d, J_{5″,6″} = 3.6) | C-1″, C-2″, C-4″, C-6″ |
| 6″ | 110.3 | 6.03 (dd, J_{4′,6′} = 2.5) | C-1″, C-2″, C-4″, C-5″ |

Tables S4a and S4b list differential metabolites from C. elegans and C. briggsae that are more than 50-fold reduced or abolished in Cel-cest-1.2 or Cbr-cest-2 mutants compared to C. elegans wildtype (N₂) or C. briggsae wildtype (AF 16), respectively. Columns include: m/z detected in both ESI− and ESI+ mode, retention time, small molecule identifier ((SMID) at www.SMID-DB.org), predicted molecular formula, detected MS/MS fragments in ESI− and ESI+ mode, the putative molecular moieties attached to the hexose core (all entries in the list contain a putative phosphate moiety), and the abundances of each metabolite in Cel-cest1. 2 or Cbr-cest-2 compared to C. elegans wildtype (N₂) or C. briggsae wildtype (AF16), respectively (“Fold over wildtype”).

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

TABLE S4a

| | | | Species the compound was | | Predicted molecular | MS/MS (ESI−, | MS/MS (ESI+, |
|---|---|---|---|---|---|---|---|
| m/z (esi−) | m/z (esi+) | RT (min.) | detected in | (SMID) | formula | actual spectra are available at www.SMID-DB.org) | actual spectra are available at www.SMID-DB.org) |
| 602.11813 | 604.13381 | 9.30 | C. elegans | angl#10 | C26H26N3O12P | Previously reported (Le et. al. elife | Previously reported (Le et. al. elife |
| | | | C. | | | 2020) | 2020) |
| | | | briggsae | | | | |
| 483.08047 | 485.09612 | 7.47 | C. elegans | angl#101 | C19 H21 O11 N2 P | 68.92886 75.99412 78.95861 86.44891 | 53.03903 81.03357 97.02825 |
| | | | | | | 86.45158 96.96933 105.18232 | 99.04384 106.02847 109.02809 |
| | | | | | | 118.74116 122.02512 127.43564 | 120.04414 124.03896 127.03868 |
| | | | | | | 136.04094 138.98073 150.981 204.173 | 166.04942 226.44617 226.45258 |
| | | | | | | 204.99155 222.49359 223.00197 | 228.06461 232.0594 250.07005 |
| | | | | | | 328.02383 346.03445 483.08237 | 330.03555 348.04623 369.10669 |
| | | | | | | | 387.11682 485.0932 |
| 434.08522 | 436.10087 | 8.02 | C. elegans | angl#12 | C16H22NO11P | 55.11328 73.52303 78.95864 96.96941 | |
| | | | C. | | | 97.53056 107.14806 118.73338 | |
| | | | briggsae | | | 125.19183 136.04076 179.85698 | |
| | | | | | | 203.28583 207.89612 211.08347 | |
| | | | | | | 223.00208 230.52628 241.01268 | |
| | | | | | | 297.03906 371.1355 378.06183 | |
| | | | | | | 434.0874 | |
| 482.08522 | 484.10087 | 9.36 | C. elegans | angl#161 | C20 H22 O11 N P | 53.18473 78.95853 86.60175 92.05018 | 83.04945 84.21027 94.44167 |
| | | | C. | | | 96.96928 121.0298 136.04065 | 105.18903 120.96615 124.49483 |
| | | | briggsae | | | 148.9648 162.98062 200.99626 | 124.49762 135.11081 149.02113 |
| | | | | | | 203.34306 204.99091 223.00185 | 175.07291 191.20917 226.1945 |
| | | | | | | 227.4801 241.01273 327.0282 | 226.2018 231.06287 249.07291 |
| | | | | | | 345.03894 345.06207 360.04993 | 304.04587 304.0545 329.0394 |
| | | | | | | 482.08679 | 347.04959 484.09805 |
| 553.12234 | 555.13799 | 9.64 | C. | angl#19 | C23 H27 O12 N2 P | 50.89142 57.46263 78.95864 84.20367 | 68.93572 84.21196 88.36558 |
| | | | briggsae | | | 86.61111 96.9694 122.02511 | 105.1914 105.19326 106.02848 |
| | | | | | | 148.96504 162.98088 203.15678 | 107.12341 118.75068 118.75323 |
| | | | | | | 204.99139 223.00197 223.04724 | 123.60458 124.03886 127.03869 |
| | | | | | | 223.06075 293.04391 328.02396 | 135.11436 155.10562 179.88504 |
| | | | | | | 346.03476 416.07672 483.08246 | 250.07005 305.63766 418.0882 |
| | | | | | | 553.12427 | 433.67294 557.00049 |
| 553.12234 | 555.13799 | 10.38 | C. | angl#21 | C23 H27 O12 N2 P | 50.8924 73.02897 78.94557 78.95865 | 75.99663 79.86706 84.20668 |
| | | | briggsae | | | 93.81509 96.9695 105.17754 | 91.56226 93.82279 105.1853 |
| | | | | | | 118.73695 136.04092 144.23221 | 118.74421 120.04404 138.80722 |
| | | | | | | 148.96495 162.98041 179.85692 | 141.89726 155.09439 177.03923 |
| | | | | | | 180.16898 191.84572 197.99667 | 179.87273 205.77977 276.67441 |
| | | | | | | 223.30037 279.02856 333.59656 | 304.04163 304.05231 315.50659 |
| | | | | | | 416.07648 | 418.08841 432.59991 |
| 566.14274 | 568.15839 | 12.17 | C. elegans | angl#22 | C25 H30 O12 N P | 77.87598 78.95863 86.60992 93.82291 | |
| | | | | | | 96.96934 101.06075 107.17558 | |
| | | | | | | 121.02978 136.04073 138.94345 | |
| | | | | | | 148.96507 158.94281 162.98087 | |
| | | | | | | 203.1548 249.68927 307.05988 | |
| | | | | | | 325.06631 444.10794 453.6312 | |
| | | | | | | 566.14539 | |
| 559.11177 | 561.12742 | 10.24 | C. | angl#23 | C25 H25 O11 N2 P | | 78.18232 84.20251 94.54453 |
| | | | briggsae | | | | 105.18025 106.02847 107.12523 |
| | | | | | | | 120.04407 124.03893 156.58569 |
| | | | | | | | 166.04947 179.86533 183.99858 |
| | | | | | | | 231.80411 303.97772 330.03604 |
| | | | | | | | 369.10583 463.14816 467.08347 |
| | | | | | | | 497.25323 561.12482 |
| 559.11177 | 561.12742 | 12.73 | C. elegans | angl#24 | C25 H25 O11 N2 P | | 55.05462 83.04917 92.04932 |
| | | | C. | | | | 118.0647 120.04401 138.05453 |
| | | | briggsae | | | | 202.08562 204.06465 209.0802 |
| | | | | | | | 226.08543 228.06464 246.0748 |
| | | | | | | | 304.04465 307.0567 326.13733 |
| | | | | | | | 328.11649 426.09305 444.10327 |
| | | | | | | | 463.18417 561.16028 |
| 579.13799 | 581.15364 | 11.53 | C. elegans | angl#26 | C25 H29 O12 N2 P | 50.20396 78.95855 92.05023 94.39323 | 74.31913 75.99399 83.04922 |
| | | | C. | | | 96.96928 99.04488 105.17025 | 105.18291 120.04408 123.54839 |
| | | | briggsae | | | 107.86208 120.96989 136.04068 | 123.551 132.88951 135.10095 |
| | | | | | | 148.95807 148.96478 162.98055 | 149.66103 202.0856 204.06508 |
| | | | | | | 197.99687 204.99136 223.00246 | 223.11235 223.11896 304.04758 |
| | | | | | | 305.04401 323.05441 442.0925 | 328.11639 408.6756 444.10355 |
| | | | | | | 579.14001 | 512.65448 524.62775 |
| 587.10669 | 589.12234 | 10.18 | C. elegans | angl#27 | C26H25N2O12P | 74.91051 78.95849 84.19585 86.45112 | 68.93347 75.99895 84.20908 |
| | | | C. | | | 94.41507 96.96925 118.72621 | 93.82517 93.82659 105.03329 |
| | | | briggsae | | | 121.02961 122.02502 136.04074 | 105.18945 106.02853 107.12599 |
| | | | | | | 148.9648 155.07069 162.98027 | 113.89217 118.7487 124.03897 |
| | | | | | | 179.56291 197.99718 204.99101 | 135.10902 155.09921 330.0358 |
| | | | | | | 328.02356 450.06198 465.07108 | 354.09546 452.07251 576.82117 |
| | | | | | | 587.10864 | 614.31366 615.34583 |
| 587.10669 | 589.12234 | 10.83 | C. elegans | angl#28 | C26H25N2O12P | 78.95863 92.05022 96.96935 | 55.0546 83.04915 84.20434 |
| | | | C. | | | 105.18637 118.74556 121.02991 | 93.82005 103.54463 105.03326 |
| | | | briggsae | | | 122.02509 136.04095 148.965 | 106.02843 118.74059 120.08041 |
| | | | | | | 156.59256 162.9803 205.65794 | 120.66382 179.87024 211.09792 |
| | | | | | | 227.45419 327.02905 345.04102 | 226.08543 230.8382 249.51474 |
| | | | | | | 400.20294 464.07581 476.78226 | 319.6174 354.09518 589.19202 |
| | | | | | | 587.10956 587.18268 | 589.30701 609.51318 |
| 587.10669 | 589.12234 | 10.60 | C. elegans | angl#29 | C26 H25 N2 O12 P | 74.91051 78.95849 84.19585 86.45112 | |
| | | | C. | | | 94.41507 96.96925 118.72621 | |
| | | | briggsae | | | 121.02961 122.02502 136.04074 | |
| | | | | | | 148.9648 155.07069 162.98027 | |
| | | | | | | 179.56291 197.99718 204.99101 | |
| | | | | | | 328.02356 450.06198 465.07108 | |
| | | | | | | 587.10864 | |
| 588.10194 | 590.11759 | 8.80 | C. elegans | angl#30 | C25 H24 O12 N3 P | 78.9585 86.60199 94.41492 96.9692 | 106.02853 118.74112 120.86057 |
| | | | C. | | | 105.17761 110.55257 122.02499 | 123.69082 124.03899 135.10225 |
| | | | briggsae | | | 124.46205 124.46684 136.04059 | 166.07195 179.86922 194.00952 |
| | | | | | | 139.52058 145.16942 148.96486 | 213.12291 227.02855 227.03375 |
| | | | | | | 328.02356 330.59363 358.0704 | 240.97318 304.04327 304.05185 |
| | | | | | | 363.10809 465.07095 588.10388 | 328.15201 443.22321 451.67789 |
| | | | | | | 588.18726 | 453.06787 465.93893 |
| 601.12234 | 603.13799 | 11.80 | C. elegans | angl#32 | C27 H27 O12 N2 P | 78.95865 86.61032 92.05022 96.96937 | 75.99773 84.20812 84.20948 |
| | | | | | | 121.02991 136.04082 148.96501 | 93.70818 93.82639 105.03327 |
| | | | | | | 162.98064 186.98125 197.99693 | 105.18967 120.04404 137.70972 |
| | | | | | | 203.14919 203.15858 204.99129 | 179.60086 204.06477 211.10971 |
| | | | | | | 224.42845 327.02875 342.04007 | 223.2607 227.29561 228.06465 |
| | | | | | | 464.07657 479.08713 601.12506 | 304.00049 304.0498 350.10074 |
| | | | | | | 601.24634 | 438.94995 466.08746 |
| 607.16929 | 609.18494 | 7.86 | C. elegans | angl#34 | C27 H33 O12 N2 P | 78.95601 78.95853 84.19226 93.80859 | 55.05484 60.24442 60.24535 |
| | | | C. | | | 94.34036 96.9693 105.16833 | 81.03384 83.04946 92.04969 |
| | | | briggsae | | | 107.80086 107.80489 118.72353 | 93.8326 94.48609 107.96759 |
| | | | | | | 124.35527 203.33247 226.94563 | 109.02837 120.04443 124.55713 |
| | | | | | | 304.01132 337.01605 470.1228 | 145.28021 164.07051 166.08632 |
| | | | | | | 488.13098 571.09552 607.07037 | 202.0863 228.06573 346.12787 |
| | | | | | | 607.1709 | 511.20715 609.18274 |
| 616.13324 | 618.14889 | 11.44 | C. elegans | angl#36 | C27H28N3O12P | 65.10978 78.95855 92.0503 94.39781 | 75.99976 84.21004 93.8264 |
| | | | C. | | | 96.96929 118.73816 124.43974 | 107.14521 120.04405 135.11145 |
| | | | briggsae | | | 134.94603 136.0407 145.14381 | 155.1024 179.8813 180.0648 |
| | | | | | | 148.96483 155.08981 162.98085 | 204.06483 223.86281 223.86952 |
| | | | | | | 197.99695 284.97031 342.03912 | 228.06473 239.08034 246.07481 |
| | | | | | | 360.04819 371.58786 479.0881 | 290.91501 304.04767 383.12161 |
| | | | | | | 616.13556 | 480.8562 481.09836 |
| 629.15364 | 631.16929 | 8.17 | C. elegans | angl#38 | C29 H31 O12 N2 P | | 53.31216 68.93379 75.99899 |
| | | | C. | | | | 105.03329 105.18906 105.19343 |
| | | | briggsae | | | | 120.04408 141.08434 166.08598 |
| | | | | | | | 178.07822 244.14021 294.31165 |
| | | | | | | | 353.46558 427.36664 427.38336 |
| | | | | | | | 497.85693 533.18964 597.25226 |
| | | | | | | | 631.16699 631.19318 |
| 679.26319 | 681.27884 | 9.07 | C. elegans | angl#40 | C32 H45 O12 N2 P | 75.98196 78.95863 86.61301 96.9694 | 75.99269 92.04936 118.73902 |
| | | | C. | | | 107.15337 107.15746 117.1961 | 120.04408 121.06437 123.11648 |
| | | | briggsae | | | 118.72307 123.61918 134.50253 | 127.03867 138.05464 146.02319 |
| | | | | | | 136.04059 168.55255 199.13477 | 147.11658 155.08672 184.04561 |
| | | | | | | 203.1563 225.11713 234.36116 | 204.06497 228.06462 251.21707 |
| | | | | | | 360.08755 542.21619 640.95776 | 257.12741 326.78384 583.29919 |
| | | | | | | 679.26782 | 678.90601 681.27338 |
| 497.09612 | 499.11177 | 8.87 | C. elegans | angl#401 | C20H23N2O11P | 52.65234 78.95853 86.60222 86.60497 | 93.6721 93.68391 105.18133 |
| | | | C. | | | 92.05006 96.96926 118.73633 | 107.04802 107.05031 118.73999 |
| | | | briggsae | | | 120.96974 136.0407 148.96478 | 120.04405 133.89235 135.09967 |
| | | | | | | 162.98024 203.34169 204.99088 | 138.05457 155.08922 179.86676 |
| | | | | | | 223.00188 225.22527 230.29785 | 203.7803 231.78969 264.0856 |
| | | | | | | 241.01236 247.77124 360.05014 | 303.97552 350.98471 362.06183 |
| | | | | | | 497.09784 | 380.1702 441.27118 |
| 679.26319 | 681.27884 | 9.20 | C. elegans | angl#41 | C32 H45 O12 N2 P | | 81.03355 81.06994 85.06487 |
| | | | C. | | | | 92.04941 109.02805 120.04405 |
| | | | briggsae | | | | 121.06459 123.11639 138.0547 |
| | | | | | | | 138.09085 147.11635 165.12685 |
| | | | | | | | 204.06488 228.0647 257.12762 |
| | | | | | | | 291.15781 302.17389 446.21475 |
| | | | | | | | 583.29852 681.27399 |
| 564.12709 | 566.14274 | 11.60 | C. elegans | angl#42 | C25 H28 O12 N P | 78.95853 86.60085 92.05001 96.96928 | |
| | | | | | | 99.04485 121.02979 136.04065 | |
| | | | | | | 148.96481 162.9807 197.9967 | |
| | | | | | | 203.34251 204.99136 223.00198 | |
| | | | | | | 224.14325 305.04391 323.05478 | |
| | | | | | | 409.0705 427.0817 442.09177 | |
| | | | | | | 564.12891 | |
| 583.13290 | 585.14855 | 9.62 | C. | angl#44 | C24 H29 O13 N2 P | 50.09505 73.02892 78.9586 84.20045 | 61.64068 75.99677 84.2078 |
| | | | briggsae | | | 92.05008 93.67819 96.96935 | 105.18643 107.13677 117.81792 |
| | | | | | | 103.03996 105.17918 120.96992 | 118.74478 120.04409 123.60245 |
| | | | | | | 123.49297 136.0408 148.96504 | 133.73776 135.10605 155.09827 |
| | | | | | | 162.98036 203.1588 204.99136 | 179.87518 198.11525 204.06517 |
| | | | | | | 309.03876 327.05032 446.08755 | 218.3665 231.80191 278.3902 |
| | | | | | | 583.13556 | 350.71692 427.5726 |
| 448.10087 | 450.11652 | 8.64 | C. elegans | angl#46 | C17 H24 O11 N P | 54.48128 78.57259 78.95866 93.8249 | |
| | | | C. | | | 96.96943 105.18742 118.74699 | |
| | | | briggsae | | | 121.14392 136.04079 148.96481 | |
| | | | | | | 155.09731 166.67833 167.01215 | |
| | | | | | | 203.02022 223.00209 241.01257 | |
| | | | | | | 293.04416 311.05478 366.98615 | |
| | | | | | | 448.10287 | |
| 617.11725 | 619.13290 | 10.37 | C. elegans | angl#47 | C27 H27 O13 N2 P | 75.99303 78.95863 86.45496 93.03432 | |
| | | | C. | | | 96.96935 105.18048 107.1311 | |
| | | | briggsae | | | 136.04082 137.02498 148.96512 | |
| | | | | | | 162.98059 197.99707 204.99144 | |
| | | | | | | 223.00198 236.41745 317.39941 | |
| | | | | | | 343.02365 360.05014 480.07275 | |
| | | | | | | 617.12146 | |
| 617.11725 | 619.13290 | 9.60 | C. elegans | angl#48 | C27 H27 O13 N2 P | 78.95868 86.4497 86.45301 92.05064 | 60.34273 66.88353 93.77401 |
| | | | C. | | | 93.82107 96.96941 105.18487 | 106.02857 120.04411 123.61794 |
| | | | briggsae | | | 124.42866 135.10248 136.04085 | 180.06493 195.46721 199.28839 |
| | | | | | | 137.02582 148.96523 203.02267 | 204.06454 228.06467 238.14255 |
| | | | | | | 223.0033 342.04175 357.0416 | 239.08066 278.37872 344.05139 |
| | | | | | | 480.07162 494.08832 617.12115 | 383.12231 472.70862 481.09915 |
| | | | | | | 617.2558 | 575.35004 619.12958 |
| 631.13290 | 633.14855 | 10.44 | C. elegans | angl#50 | C28 H29 O13 N2 P | 78.95866 86.61131 92.0503 96.96939 | 52.77689 68.93279 81.01479 |
| | | | C. | | | 107.05044 121.03011 136.04083 | 84.20921 97.92274 107.04887 |
| | | | briggsae | | | 148.96498 151.04053 162.9809 | 107.16924 118.74797 120.04409 |
| | | | | | | 197.99698 203.15681 204.99141 | 155.10025 204.06496 223.39807 |
| | | | | | | 223.00209 342.04007 357.03943 | 223.40353 228.06467 254.08003 |
| | | | | | | 360.0502 494.08832 497.09827 | 304.05069 380.11176 496.09915 |
| | | | | | | 631.13721 | 640.96454 640.99194 |
| 590.11759 | 592.13324 | 11.11 | C. elegans | angl#51 | C25 H26 O12 N3 P | 57.46047 66.03432 78.95865 84.20077 | 84.20779 93.71883 93.82466 |
| | | | C. | | | 95.13375 96.96943 110.0211 | 94.02869 104.10679 105.18801 |
| | | | briggsae | | | 110.02511 118.73528 135.09471 | 107.08305 107.09337 118.74601 |
| | | | | | | 136.04097 148.9651 162.98077 | 120.04405 135.10768 155.09903 |
| | | | | | | 179.86285 197.9968 204.99153 | 184.07306 184.59518 223.86838 |
| | | | | | | 223.00253 316.02414 360.05188 | 223.87619 223.88097 304.05258 |
| | | | | | | 453.07236 | 339.0961 455.08463 |
| 590.11759 | 592.13324 | 10.84 | C. elegans | angl#52 | C25 H26 O12 N3 P | 66.03434 78.95863 92.05013 93.82376 | 84.20779 93.71883 93.82466 |
| | | | C. | | | 96.96935 105.18472 110.02507 | 94.02869 104.10679 105.18801 |
| | | | briggsae | | | 118.74602 136.04092 148.96507 | 107.08305 107.09337 118.74601 |
| | | | | | | 162.98087 223.00247 223.84451 | 120.04405 135.10768 155.09903 |
| | | | | | | 223.85722 304.00629 316.02402 | 184.07306 184.59518 223.86838 |
| | | | | | | 334.03583 360.05078 453.07208 | 223.87619 223.88097 304.05258 |
| | | | | | | 590.12048 | 455.08463 579.71161 |
| 544.15839 | 546.17404 | 11.59 | C. | angl#54 | C23 H32 O12 N P | 53.40808 78.9585 86.59651 87.28533 | 93.83659 94.02892 105.20023 |
| | | | briggsae | | | 93.81419 96.96939 105.17729 | 118.76237 124.11217 135.12463 |
| | | | | | | 118.73257 118.73666 124.50652 | 142.12285 170.15398 184.07372 |
| | | | | | | 135.09267 145.2162 211.08199 | 184.16946 202.10736 226.31151 |
| | | | | | | 227.86993 358.07101 416.40057 | 302.19592 357.23795 407.28989 |
| | | | | | | 426.04758 476.11264 492.06024 | 425.30023 456.31906 457.32663 |
| | | | | | | 544.10205 | 485.32172 546.3385 |
| 615.13799 | 617.15364 | 11.72 | C. | angl#56 | C28H29N2O12P | 78.95865 86.61275 91.05498 92.05029 | 84.2121 93.74004 107.11538 |
| | | | briggsae | | | 96.96934 107.17877 123.64606 | 107.11755 120.04405 123.5688 |
| | | | | | | 135.04578 136.04076 148.96498 | 123.57163 135.11412 155.10469 |
| | | | | | | 162.9808 197.99727 204.99142 | 204.06483 230.4552 232.27657 |
| | | | | | | 223.00186 224.43748 341.04459 | 244.97278 270.96182 295.77274 |
| | | | | | | 360.0506 478.09286 615.14178 | 308.13034 344.3306 375.01569 |
| | | | | | | 640.96216 | 480.1026 551.31396 |
| 460.10087 | 462.11652 | 9.16 | C. elegans | angl#601 | C18 H24 O11 N P | 54.02916 78.95851 86.60173 94.40849 | 53.20533 75.98508 84.19356 |
| | | | C. | | | 96.96922 99.04478 125.87339 | 105.17049 119.64037 120.04158 |
| | | | briggsae | | | 136.0406 148.96474 157.86316 | 120.04405 135.08421 146.74179 |
| | | | | | | 192.9908 223.00166 227.33609 | 155.07115 157.76543 222.15416 |
| | | | | | | 241.01227 305.04401 323.05417 | 223.34396 223.35085 264.08533 |
| | | | | | | 378.0603 415.03839 460.03827 | 304.05032 312.84796 362.06207 |
| | | | | | | 460.10229 | 414.12378 462.20139 |
| 586.11144 | 588.12709 | 11.87 | C. elegans | angl#70 | C27 H26 N O12 P | 50.04332 78.95851 96.96928 | |
| | | | C. | | | 118.73649 121.02977 136.04083 | |
| | | | briggsae | | | 148.96478 161.7142 162.98065 | |
| | | | | | | 176.79967 203.34651 222.8712 | |
| | | | | | | 304.01285 327.02826 345.03897 | |
| | | | | | | 396.08655 449.06497 464.07584 | |
| | | | | | | 518.12073 586.11328 | |
| 560.09579 | 562.11144 | 11.53 | C. elegans | bzglu#10 | C25 H24 O12 N P | 50.72117 50.72234 78.95853 94.42484 | |
| | | | | | | 96.96926 105.1731 110.02522 | |
| | | | | | | 121.02975 135.08955 148.96463 | |
| | | | | | | 162.98117 203.3425 222.72235 | |
| | | | | | | 223.00128 304.00842 304.02661 | |
| | | | | | | 327.02777 345.03882 438.06226 | |
| | | | | | | 560.09747 | |
| 561.09104 | 563.10669 | 9.58 | C. elegans | bzglu#12 | C24 H23 O12 N2 P | | 84.21037 94.46944 94.47182 |
| | | | C. | | | | 94.47527 105.03353 106.02867 |
| | | | briggsae | | | | 107.951 107.95328 109.82473 |
| | | | | | | | 118.74548 124.53848 145.2578 |
| | | | | | | | 145.26122 178.37459 205.86708 |
| | | | | | | | 211.11125 224.16484 249.44727 |
| | | | | | | | 406.08179 452.07333 |
| 561.09104 | 563.10669 | 10.23 | C. elegans | bzglu#13 | C24 H23 O12 N2 P | | 57.46389 62.80637 75.9961 |
| | | | | | | | 93.82291 105.18502 118.74357 |
| | | | | | | | 121.03937 130.77776 136.49512 |
| | | | | | | | 141.1284 155.09474 159.83893 |
| | | | | | | | 170.34326 181.06047 230.32031 |
| | | | | | | | 232.86993 251.22913 319.59445 |
| | | | | | | | 341.39539 447.11453 |
| 445.08997 | 447.10562 | 9.62 | C. elegans | bzglu#201 | C18 H23 O11 P | 54.65283 66.52367 75.98717 78.95853 | |
| | | | C. | | | 86.59713 95.2371 96.96926 105.42665 | |
| | | | briggsae | | | 121.02982 148.96483 162.98077 | |
| | | | | | | 163.86372 200.99651 203.32895 | |
| | | | | | | 223.00185 241.01253 323.05441 | |
| | | | | | | 327.02814 345.03928 445.09149 | |
| 552.12709 | 554.14274 | 8.98 | C. elegans | bzglu#4 | C24 H28 O12 N P | | 55.05485 75.99854 78.44279 |
| | | | | | | | 83.04945 84.2083 84.20955 |
| | | | | | | | 85.85622 93.82543 105.18757 |
| | | | | | | | 105.18931 106.02885 108.03045 |
| | | | | | | | 118.74809 135.10797 198.2657 |
| | | | | | | | 223.37898 223.38594 257.73956 |
| | | | | | | | 332.11206 430.08994 |
| 573.09104 | 575.10669 | 10.87 | C. elegans | bzglu#6 | C25H23N2O12P | 50.37834 60.43568 78.95862 93.69283 | 54.47928 105.03327 105.18517 |
| | | | C. | | | 96.96936 105.18089 107.06194 | 105.18753 106.0285 107.11108 |
| | | | briggsae | | | 118.73739 121.03002 122.02508 | 109.02816 118.74522 124.03653 |
| | | | | | | 148.96497 162.98039 179.86661 | 124.03896 135.1068 135.10941 |
| | | | | | | 203.15988 204.99162 230.41316 | 155.09784 179.8781 223.36221 |
| | | | | | | 255.4989 328.02386 450.06207 | 304.04068 304.05096 330.03564 |
| | | | | | | 572.09888 | 354.09558 452.07248 |
| 551.13184 | 553.14749 | 10.15 | C. elegans | bzglu#8 | C25 H29 O12 P | 50.96988 78.95851 79.23584 79.40337 | |
| | | | | | | 86.6011 94.44073 96.96926 99.04477 | |
| | | | | | | 107.91904 121.03013 123.04546 | |
| | | | | | | 155.0705 156.39014 160.32196 | |
| | | | | | | 203.33809 209.86107 228.45087 | |
| | | | | | | 388.10339 429.09677 551.13373 | |
| 549.09104 | 551.10669 | 10.63 | C. elegans | bzglu#9 | C23 H23 O12 N2 P | 78.95853 93.81328 96.96928 | |
| | | | C. | | | 110.02502 118.73462 135.0943 | |
| | | | briggsae | | | 135.89751 145.25218 148.9648 | |
| | | | | | | 158.31697 222.98035 223.00204 | |
| | | | | | | 227.42886 304.00421 304.02301 | |
| | | | | | | 316.02322 334.03397 427.05548 | |
| | | | | | | 511.1409 549.0929 | |
| 734.22138 | 736.23703 | 10.88 | C. elegans | higlas#7 | C34 H42 O15 N P | 78.95851 86.60098 93.80782 94.50877 | |
| | | | | | | 96.96925 105.16888 107.99517 | |
| | | | | | | 118.72562 155.06996 174.75633 | |
| | | | | | | 203.33847 224.12323 273.13516 | |
| | | | | | | 304.0116 356.05484 612.18713 | |
| | | | | | | 638.9635 717.51056 717.58478 | |
| | | | | | | 734.22369 | |
| 583.11177 | 585.12742 | 10.28 | C. elegans | higlu#3 | C27H25N2O11P | 78.95853 79.6107 84.2002 86.60039 | 81.03364 93.8229 105.03326 |
| | | | C. | | | 93.81461 96.96931 104.1823 | 106.02847 109.02814 118.74532 |
| | | | briggsae | | | 105.17554 107.97623 107.98085 | 121.02806 124.03896 166.04933 |
| | | | | | | 122.02507 124.56419 124.56999 | 179.87532 223.46507 226.08505 |
| | | | | | | 203.34386 239.14767 316.57379 | 304.05127 330.0358 349.11676 |
| | | | | | | 445.08093 461.07574 463.09241 | 452.07233 465.1037 468.06763 |
| | | | | | | 583.11334 | 487.14801 585.12402 |
| 583.11177 | 585.12742 | 10.85 | C. elegans | higlu#4 | C27H25N2O11P | 50.11848 73.53172 78.95853 86.60017 | 78.03458 78.95863 80.33524 |
| | | | | | | 96.96925 107.95139 122.02504 | 86.61156 92.05051 96.96936 |
| | | | | | | 123.0284 136.04059 137.04428 | 105.1959 122.02515 123.02267 |
| | | | | | | 197.99637 203.33989 226.16963 | 123.02846 136.04083 137.04427 |
| | | | | | | 322.04965 323.05286 445.08224 | 197.99699 225.87064 322.05023 |
| | | | | | | 446.08511 463.09192 464.09607 | 323.05371 445.08182 446.08536 |
| | | | | | | 583.13281 | 464.0947 583.13367 |
| 720.24212 | 722.25777 | 11.80 | C. elegans | iglas#101 | C34 H44 O14 N P | 75.98959 78.95853 94.41531 96.96946 | |
| | | | | | | 105.17619 107.88396 124.45963 | |
| | | | | | | 161.83398 179.95119 205.74577 | |
| | | | | | | 218.70569 228.4581 275.15231 | |
| | | | | | | 550.1864 598.20685 638.95215 | |
| | | | | | | 663.41992 717.47913 717.55206 | |
| | | | | | | 720.24463 | |
| 746.25777 | 748.27342 | 12.48 | C. elegans | iglas#301 | C36 H46 O14 N P | 78.95854 86.60203 93.81118 96.96928 | |
| | | | | | | 105.17189 107.92922 124.51595 | |
| | | | | | | 203.34187 281.24982 282.25171 | |
| | | | | | | 301.16608 322.0502 340.05927 | |
| | | | | | | 422.58545 624.22217 640.51172 | |
| | | | | | | 717.5174 717.57471 746.26038 | |
| | | | | | | 746.56201 | |
| 733.23737 | 735.25302 | 11.36 | C. elegans | iglas#701 | C34 H43 O14 N2 P | 73.02897 78.95856 86.60087 96.96931 | |
| | | | | | | 105.18009 124.4492 201.53618 | |
| | | | | | | 203.34331 225.32541 267.233 | |
| | | | | | | 273.13559 304.02118 322.04996 | |
| | | | | | | 340.0603 596.19269 638.92316 | |
| | | | | | | 638.97296 717.47223 717.54889 | |
| | | | | | | 733.23999 | |
| 718.22647 | 720.24212 | 11.63 | C. elegans | iglas#702 | C34 H42 O14 N P | 73.02882 78.95849 86.6012 93.8112 | |
| | | | | | | 94.43188 96.96925 107.90531 | |
| | | | | | | 179.47174 203.3434 222.73381 | |
| | | | | | | 269.2489 304.00757 304.02744 | |
| | | | | | | 322.04865 340.05978 596.19189 | |
| | | | | | | 638.97485 717.54083 718.22839 | |
| | | | | | | 718.54132 | |
| 451.09064 | 453.10629 | 9.48 | C. elegans | iglu#101 | C19H21N2O9P | 54.39986 60.38681 73.60184 78.95854 | 69.03402 81.03386 85.02872 |
| | | | C. | | | 93.81931 96.96928 100.14008 | 94.02898 112.03937 118.06521 |
| | | | briggsae | | | 105.18227 107.8572 107.86152 | 127.03912 178.04987 184.07593 |
| | | | | | | 125.01723 146.08636 224.89441 | 211.0864 225.01582 226.08644 |
| | | | | | | 230.50409 233.40883 235.22231 | 226.09875 226.10428 238.07085 |
| | | | | | | 304.01859 340.0593 358.07053 | 244.0968 304.05429 336.04767 |
| | | | | | | 451.09213 | 355.12799 453.10556 |
| 462.09539 | 464.11104 | 10.28 | C. elegans | iglu#121 | C21H22NO9P | 53.94945 73.49477 78.95854 86.60292 | 68.93686 91.72733 93.83044 |
| | | | C. | | | 93.62287 96.96928 105.16904 | 105.03361 105.19332 118.06512 |
| | | | briggsae | | | 118.72594 121.02971 124.51448 | 126.73933 135.11552 155.10915 |
| | | | | | | 124.52031 152.99637 180.99124 | 179.88879 224.49088 224.4984 |
| | | | | | | 203.34544 223.00215 229.50745 | 224.50452 225.01585 244.09642 |
| | | | | | | 307.05945 325.07077 340.05984 | 249.07541 251.25075 278.0314 |
| | | | | | | 462.09601 | 304.05209 373.2316 |
| 442.12669 | 444.14234 | 10.54 | C. elegans | iglu#141 | C19 H26 O9 N P | 54.78176 54.78316 78.95853 86.59731 | 57.07049 69.034 81.03383 |
| | | | C. | | | 94.38846 96.96978 105.16628 | 85.06513 97.02855 99.04417 |
| | | | briggsae | | | 106.71937 107.85747 181.83965 | 109.02845 118.06513 127.03904 |
| | | | | | | 203.34148 222.68468 305.04593 | 154.06519 172.0757 198.09142 |
| | | | | | | 315.4147 340.06134 358.07037 | 202.086 211.09634 226.08607 |
| | | | | | | 405.92813 441.90524 442.095 | 229.10707 244.09654 309.07281 |
| | | | | | | 442.12753 | 328.15378 346.16455 |
| 476.11104 | 478.12669 | 10.78 | C. elegans | iglu#16 | C22 H24 O9 N P | 53.40666 59.33614 78.95856 88.80988 | |
| | | | C. | | | 94.46926 96.96914 105.1736 | |
| | | | briggsae | | | 107.94904 107.95299 124.53297 | |
| | | | | | | 145.25558 223.08147 256.78922 | |
| | | | | | | 279.23352 304.02118 340.05969 | |
| | | | | | | 358.0705 476.11249 476.24207 | |
| | | | | | | 476.2785 | |
| 478.09031 | 480.10596 | 9.31 | C. elegans | iglu#181 | C21 H22 O10 N P | 53.40666 59.33614 78.95856 88.80988 | 84.21514 85.0288 94.41797 |
| | | | | | | 94.46926 96.96914 105.1736 | 94.41964 107.8905 107.89254 |
| | | | | | | 107.94904 107.95299 124.53297 | 109.02841 118.06505 121.02849 |
| | | | | | | 145.25558 223.08147 256.78922 | 124.46664 139.03909 155.11066 |
| | | | | | | 279.23352 304.02118 340.05969 | 184.07565 225.01573 226.08601 |
| | | | | | | 358.0705 476.11249 476.24207 | 244.09662 251.34599 265.07025 |
| | | | | | | 476.2785 | 363.04684 382.12836 |
| 519.11686 | 521.13251 | 11.18 | C. elegans | iglu#19 | C23 H25 O10 N2 P | 75.98148 78.95849 94.39281 96.96947 | |
| | | | | | | 105.16602 107.85878 107.86346 | |
| | | | | | | 110.02538 124.43221 124.43712 | |
| | | | | | | 202.78258 223.37059 223.38385 | |
| | | | | | | 340.06253 358.07278 380.05438 | |
| | | | | | | 451.09213 473.07428 505.46701 | |
| | | | | | | 519.07904 | |
| 519.11686 | 521.13251 | 10.31 | C. elegans | iglu#21 | C23 H25 O10 N2 P | 75.98148 78.95849 94.39281 96.96947 | |
| | | | C. | | | 105.16602 107.85878 107.86346 | |
| | | | briggsae | | | 110.02538 124.43221 124.43712 | |
| | | | | | | 202.78258 223.37059 223.38385 | |
| | | | | | | 340.06253 358.07278 380.05438 | |
| | | | | | | 451.09213 473.07428 505.46701 | |
| | | | | | | 519.07904 | |
| 533.13251 | 535.14816 | 12.29 | C. elegans | iglu#22 | C24 H27 O10 N2 P | 51.49329 78.95853 84.1997 86.60059 | 55.05483 83.04945 94.02893 |
| | | | | | | 94.39445 94.39823 96.96925 99.04475 | 94.4282 107.90025 107.90236 |
| | | | | | | 111.64845 118.73367 124.43331 | 109.02847 118.06522 124.48047 |
| | | | | | | 153.77753 155.08096 203.34151 | 143.07039 155.1093 209.0807 |
| | | | | | | 222.99898 304.01572 422.10281 | 226.08606 302.10165 307.05756 |
| | | | | | | 440.07761 440.11249 533.13416 | 320.11212 326.13821 400.07849 |
| | | | | | | | 418.08899 535.14667 |
| 519.11686 | 521.13251 | 10.52 | C. elegans | iglu#23 | C23 H25 O10 N2 P | | 57.03414 76.17534 94.53166 |
| | | | | | | | 106.02883 108.0203 109.02853 |
| | | | | | | | 118.06509 124.0394 124.61653 |
| | | | | | | | 126.35994 145.35056 145.3539 |
| | | | | | | | 179.89293 190.04988 205.99762 |
| | | | | | | | 232.06035 306.09708 403.75125 |
| | | | | | | | 404.07413 521.13104 |
| 533.13251 | 535.14816 | 11.85 | C. elegans | iglu#24 | C24 H27 O10 N2 P | 73.02895 75.98453 78.95853 84.19275 | |
| | | | C. | | | 86.60114 94.40417 94.40823 96.96926 | |
| | | | briggsae | | | 102.05298 105.16676 124.45467 | |
| | | | | | | 124.45963 136.04089 155.06868 | |
| | | | | | | 184.20874 304.01474 347.76083 | |
| | | | | | | 372.5386 396.08624 533.1344 | |
| 519.11686 | 521.13251 | 10.89 | C. elegans | iglu#25 | C23 H25 O10 N2 P | | 57.03414 76.17534 94.53166 |
| | | | | | | | 106.02883 108.0203 109.02853 |
| | | | | | | | 118.06509 124.0394 124.61653 |
| | | | | | | | 126.35994 145.35056 145.3539 |
| | | | | | | | 179.89293 190.04988 205.99762 |
| | | | | | | | 232.06035 306.09708 403.75125 |
| | | | | | | | 404.07413 521.13104 |
| 544.11211 | 546.12776 | 11.53 | C. elegans | iglu#27 | C24 H24 O10 N3 P | 75.98491 78.95853 84.19361 86.60133 | 51.12113 118.75488 124.11216 |
| | | | | | | 96.96926 105.16934 107.86297 | 135.1167 142.12277 155.10852 |
| | | | | | | 107.86709 118.72672 124.43195 | 170.15398 184.07327 184.16959 |
| | | | | | | 124.43766 236.79739 251.1877 | 202.10751 224.55295 224.55942 |
| | | | | | | 268.35016 356.19739 358.07059 | 302.19577 304.05359 357.2381 |
| | | | | | | 426.0448 451.09592 476.11258 | 407.28967 425.30035 457.32703 |
| | | | | | | 544.11157 | 485.32181 546.33844 |
| 544.13726 | 546.15291 | 13.04 | C. elegans | iglu#28 | C26 H28 O10 N P | 51.16898 78.95853 86.5993 86.60188 | 55.05483 68.93397 83.04945 |
| | | | C. | | | 96.96928 99.04489 107.91918 | 105.03358 109.02845 118.06513 |
| | | | briggsae | | | 116.05099 121.02992 203.34659 | 118.74865 118.75073 135.11131 |
| | | | | | | 222.72763 279.23364 304.00839 | 143.07043 155.10162 211.11279 |
| | | | | | | 304.02869 322.04984 340.05927 | 224.67909 224.6906 304.04861 |
| | | | | | | 422.10214 476.28 544.13934 | 307.05728 313.10669 326.13718 |
| | | | | | | 544.26782 | 331.11765 411.08359 |
| 545.13251 | 547.14816 | 11.02 | C. elegans | iglu#30 | C25 H27 O10 N2 P | 51.14044 54.72091 78.95853 86.60078 | 55.05485 81.03387 83.04948 |
| | | | | | | 93.81094 96.9659 96.96925 105.17319 | 106.02884 109.02846 118.06515 |
| | | | | | | 122.02499 124.54322 203.33745 | 124.03938 166.04994 208.07545 |
| | | | | | | 203.34726 223.42888 251.58308 | 225.15787 226.08601 232.06026 |
| | | | | | | 278.05588 304.01822 445.08157 | 304.05173 314.10129 330.03714 |
| | | | | | | 463.09229 477.10822 545.13416 | 332.11279 349.11783 430.08932 |
| | | | | | | | 449.1702 547.14764 |
| 545.13251 | 547.14816 | 11.84 | C. elegans | iglu#31 | C25 H27 O10 N2 P | 78.95852 79.93417 84.19633 89.70659 | 55.05487 81.0339 83.0495 |
| | | | C. | | | 94.38783 96.96926 96.97226 | 94.02895 106.02888 109.02844 |
| | | | briggsae | | | 110.02534 118.73016 137.84738 | 118.06516 124.03945 184.07581 |
| | | | | | | 179.84981 340.06024 358.07242 | 190.04993 208.07562 226.08633 |
| | | | | | | 359.07477 422.10214 434.08847 | 232.0601 314.10208 332.11237 |
| | | | | | | 451.09525 452.09598 477.11484 | 412.07913 430.08948 449.17072 |
| | | | | | | 545.11725 | 529.13708 547.14764 |
| 546.15291 | 548.16856 | 13.40 | C. elegans | iglu#32 | C26 H30 O10 N P | 51.11089 78.95853 86.59999 96.96928 | |
| | | | | | | 101.06051 105.18182 118.73705 | |
| | | | | | | 155.78033 203.34547 223.71927 | |
| | | | | | | 270.60455 281.24921 304.01849 | |
| | | | | | | 322.04922 410.2323 424.11777 | |
| | | | | | | 478.22031 478.29388 546.15491 | |
| | | | | | | 546.28125 | |
| 546.15291 | 548.16856 | 13.58 | C. elegans | iglu#34 | C26 H30 O10 N P | 51.11089 78.95853 86.59999 96.96928 | |
| | | | | | | 101.06051 105.18182 118.73705 | |
| | | | | | | 155.78033 203.34547 223.71927 | |
| | | | | | | 270.60455 281.24921 304.01849 | |
| | | | | | | 322.04922 410.2323 424.11777 | |
| | | | | | | 478.22031 478.29388 546.15491 | |
| | | | | | | 546.28125 | |
| 547.14816 | 549.16381 | 11.81 | C. elegans | iglu#36 | C25H29N2O10P | 51.0825 62.63796 63.58872 78.95606 | |
| | | | | | | 78.95853 83.49227 94.40952 96.96934 | |
| | | | | | | 101.06049 105.17789 118.73723 | |
| | | | | | | 124.45099 124.45647 131.06131 | |
| | | | | | | 222.14005 322.04861 424.11761 | |
| | | | | | | 441.14362 459.97211 547.14996 | |
| 555.11686 | 557.13251 | 12.24 | C. elegans | iglu#37 | C26 H25 O10 N2 P | | 81.03381 85.0287 94.02898 |
| | | | | | | | 105.03362 118.06512 138.17839 |
| | | | | | | | 154.04997 154.06523 208.07561 |
| | | | | | | | 225.00269 226.08638 231.06516 |
| | | | | | | | 318.03677 319.10684 324.08612 |
| | | | | | | | 337.11777 342.09674 422.06317 |
| | | | | | | | 440.07352 459.15439 |
| 555.11686 | 557.13251 | 12.53 | C. elegans | iglu#38 | C26 H25 O10 N2 P | | 84.22446 93.84353 94.02895 |
| | | | | | | | 105.03358 105.20794 107.98866 |
| | | | | | | | 118.06519 165.05447 179.91383 |
| | | | | | | | 198.09135 211.14693 224.30119 |
| | | | | | | | 226.08611 231.06477 304.05069 |
| | | | | | | | 324.08676 329.04181 342.09659 |
| | | | | | | | 422.06305 440.07376 |
| 556.11211 | 558.12776 | 10.78 | C. elegans | iglu#39 | C25 H24 O10 N3 P | 78.95852 86.60139 93.81087 94.49577 | 81.03386 94.02899 96.04466 |
| | | | | | | 96.96924 107.97984 110.02499 | 106.02892 118.06517 124.03946 |
| | | | | | | 118.73122 155.07803 175.81754 | 166.05 202.05008 208.07574 |
| | | | | | | 179.85066 226.54314 226.55431 | 214.04991 226.08607 232.06039 |
| | | | | | | 272.08884 304.01511 319.46579 | 250.07066 325.08139 330.03711 |
| | | | | | | 340.05984 433.08139 463.09189 | 343.09213 441.06894 460.14957 |
| | | | | | | 556.11395 | 540.11646 558.12616 |
| 556.11211 | 558.12776 | 10.50 | C. elegans | iglu#40 | C25 H24 O10 N3 P | 50.81455 78.95855 86.43904 86.44549 | 81.03387 94.02897 106.02885 |
| | | | C. | | | 86.45528 93.80989 96.96931 | 109.02852 118.06516 124.03941 |
| | | | briggsae | | | 110.02509 118.72881 122.02502 | 144.0444 166.04996 198.09135 |
| | | | | | | 135.08734 141.45276 177.08992 | 226.08617 232.06038 250.07085 |
| | | | | | | 203.30995 230.68266 340.06021 | 330.03687 343.09204 348.04736 |
| | | | | | | 445.08054 463.09241 478.85696 | 349.11798 441.06897 460.15005 |
| | | | | | | 556.11383 | 465.1059 558.12653 |
| 477.10629 | 479.12194 | 9.94 | C. elegans | iglu#401 | C21H23N2O9P | 53.36987 68.31073 78.95855 86.60619 | 81.03385 92.04977 118.06521 |
| | | | C. | | | 96.9693 107.8917 124.4672 136.04063 | 119.76888 120.04446 127.03904 |
| | | | briggsae | | | 152.99629 197.99672 203.33923 | 138.05507 172.07571 184.07587 |
| | | | | | | 223.00165 224.39587 225.27356 | 204.06552 216.06548 226.08606 |
| | | | | | | 225.285 280.23795 304.01242 | 226.65025 244.09674 246.07591 |
| | | | | | | 340.05991 358.07034 477.10791 | 264.0864 362.06311 363.13345 |
| | | | | | | | 381.14423 479.12115 |
| 582.12776 | 584.14341 | 10.95 | C. elegans | iglu#41 | C27 H26 O10 N3 P | 78.95853 80.33051 85.06508 85.06772 | |
| | | | C. | | | 86.59924 86.60183 92.05019 96.96928 | |
| | | | briggsae | | | 107.96045 118.73325 122.02504 | |
| | | | | | | 136.0407 197.86435 197.99672 | |
| | | | | | | 203.34145 204.99173 322.04959 | |
| | | | | | | 445.08185 463.09213 582.12976 | |
| 582.1283 | 584.14398 | 10.49 | C. elegans | iglu#41 | C27H26N3O10P | Previously reported (Le et. al. elife | Previously reported (Le et. al. elife |
| | | | C. | | | 2020) | 2020) |
| | | | briggsae | | | | |
| 559.14871 | 561.16439 | 10.48 | C. elegans | iglu#42 | C26H29N2O10P | Previously reported (Le et. al. elife | Previously reported (Le et. al. elife |
| | | | C. | | | 2020) | 2020) |
| | | | briggsae | | | | |
| 561.16381 | 563.17946 | 13.07 | C. elegans | iglu#44 | C26 H31 O10 N2 P | 50.6907 78.95856 86.59949 94.38712 | |
| | | | | | | 96.96929 105.17963 107.85587 | |
| | | | | | | 118.73624 124.42696 155.0856 | |
| | | | | | | 160.28455 192.99165 223.00304 | |
| | | | | | | 224.79655 241.01247 304.01398 | |
| | | | | | | 433.4176 471.53922 561.17365 | |
| | | | | | | 561.24664 | |
| 562.15906 | 564.17471 | 9.79 | C. elegans | iglu#45 | C25H30N3O10P | 57.46374 75.99708 78.95853 78.96117 | |
| | | | | | | 93.82233 96.91728 96.96937 | |
| | | | | | | 110.02516 126.5277 127.39748 | |
| | | | | | | 137.97256 147.55956 174.48755 | |
| | | | | | | 205.79216 347.05502 386.3638 | |
| | | | | | | 440.0773 469.09329 562.11359 | |
| | | | | | | 580.92572 | |
| 562.15906 | 564.17471 | 9.60 | C. elegans | iglu#46 | C25H30N3O10P | 57.46374 75.99708 78.95853 78.96117 | |
| | | | | | | 93.82233 96.91728 96.96937 | |
| | | | | | | 110.02516 126.5277 127.39748 | |
| | | | | | | 137.97256 147.55956 174.48755 | |
| | | | | | | 205.79216 347.05502 386.3638 | |
| | | | | | | 440.0773 469.09329 562.11359 | |
| | | | | | | 580.92572 | |
| 563.14307 | 565.15872 | 10.89 | C. elegans | iglu#47 | C25 H29 O11 N2 P | 50.64239 78.95851 87.41492 | |
| | | | | | | 105.17938 118.73993 124.44572 | |
| | | | | | | 124.45287 178.88483 179.88461 | |
| | | | | | | 179.89281 180.89252 180.90048 | |
| | | | | | | 181.90097 196.89532 197.89554 | |
| | | | | | | 223.41754 284.92685 304.0166 | |
| | | | | | | 527.00415 563.0246 | |
| 563.14307 | 565.15872 | 10.50 | C. | iglu#48 | C25 H29 O11 N2 P | 50.63854 78.95857 94.06632 96.96938 | |
| | | | briggsae | | | 103.03996 107.88106 118.34013 | |
| | | | | | | 118.73916 124.46281 126.88675 | |
| | | | | | | 135.12669 179.86334 212.81227 | |
| | | | | | | 216.51364 227.90973 295.97134 | |
| | | | | | | 305.49673 322.04919 426.0968 | |
| | | | | | | 563.14502 | |
| 563.14307 | 565.15872 | 11.42 | C. | iglu#49 | C25 H29 O11 N2 P | | 76.00529 84.21716 86.08596 |
| | | | briggsae | | | | 86.0918 86.09318 105.19836 |
| | | | | | | | 108.05826 118.76031 124.66534 |
| | | | | | | | 155.11571 178.09387 178.10408 |
| | | | | | | | 178.10817 178.11259 178.12419 |
| | | | | | | | 178.13043 202.29507 211.13017 |
| | | | | | | | 222.49077 222.49763 |
| 566.12161 | 568.13726 | 13.26 | C. elegans | iglu#50 | C28 H26 O10 N P | 78.95851 86.60258 96.96927 | 53.03922 105.03362 118.06527 |
| | | | C. | | | 107.96659 107.9707 118.74023 | 138.17181 138.17438 165.05478 |
| | | | briggsae | | | 121.02975 124.54919 124.55506 | 184.076 208.07579 223.96141 |
| | | | | | | 201.18199 203.34497 219.41884 | 223.96822 304.05322 329.04163 |
| | | | | | | 222.9879 301.21869 304.00885 | 330.11221 335.09094 348.12271 |
| | | | | | | 322.05008 403.43726 444.05908 | 353.10144 433.06799 451.07889 |
| | | | | | | 444.08627 566.12323 | 470.15964 568.13525 |
| 567.11686 | 569.13251 | 11.37 | C. elegans | iglu#52 | C27 H25 O10 N2 P | 50.53263 72.80424 78.95852 86.60025 | 53.03921 81.03387 85.02868 |
| | | | C. | | | 96.96927 105.17567 107.96175 | 105.03358 106.02882 109.02844 |
| | | | briggsae | | | 117.79089 121.02968 122.02504 | 118.06512 124.03938 154.06505 |
| | | | | | | 124.54747 124.55247 203.34683 | 166.04993 184.07581 208.07578 |
| | | | | | | 227.28993 227.30132 322.04886 | 226.08601 226.17216 330.03683 |
| | | | | | | 372.47934 444.0863 445.0816 | 349.11777 354.09665 452.07358 |
| | | | | | | 567.11841 | 471.15463 569.1311 |
| 567.11686 | 569.13251 | 12.10 | C. elegans | iglu#53 | C27 H25 O10 N2 P | 50.53307 54.69776 75.98828 78.95855 | 66.60043 76.00045 93.8276 |
| | | | | | | 86.60141 94.39828 96.9693 121.02982 | 94.38294 105.0336 106.02885 |
| | | | | | | 122.02546 124.43588 158.93271 | 109.02849 118.06519 124.03938 |
| | | | | | | 203.3401 203.34988 223.89584 | 135.11067 155.1032 190.05011 |
| | | | | | | 304.01178 322.05203 337.61502 | 232.06062 254.39615 336.08643 |
| | | | | | | 444.0863 451.6152 567.11847 | 348.12274 354.09677 434.06378 |
| | | | | | | | 452.07397 569.13147 |
| 567.11686 | 569.13251 | 11.73 | C. elegans | iglu#54 | C27 H25 O10 N2 P | 50.53256 73.0272 78.95852 86.5989 | 53.0392 83.04951 96.04466 |
| | | | | | | 86.60172 94.42673 96.96925 | 105.03365 105.1973 109.0285 |
| | | | | | | 107.89674 118.73138 121.02979 | 124.03944 166.05 184.07568 |
| | | | | | | 122.025 124.47859 135.08977 | 208.07561 225.51868 225.52609 |
| | | | | | | 203.34637 222.71198 304.00995 | 231.06506 336.08612 349.11838 |
| | | | | | | 304.02792 322.04913 445.08151 | 354.0968 452.07339 471.15387 |
| | | | | | | 567.11865 | 551.12152 569.1311 |
| 568.11211 | 570.12776 | 9.93 | C. elegans | iglu#56 | C26H24N3O10P | 50.50622 53.99556 54.65442 59.6573 | 78.03423 81.03384 106.02885 |
| | | | C. | | | 78.95855 86.60081 96.96928 | 109.02855 118.06519 124.03942 |
| | | | briggsae | | | 113.17274 122.02502 124.51893 | 136.69994 166.04997 184.07568 |
| | | | | | | 155.07457 203.33702 203.34619 | 190.04991 225.31029 226.08609 |
| | | | | | | 205.83356 228.39136 261.80856 | 232.06018 330.03693 337.08154 |
| | | | | | | 322.04916 445.08157 562.30011 | 349.11768 355.09164 453.06897 |
| | | | | | | 568.11389 | 472.15042 570.12646 |
| 570.12776 | 572.14341 | 12.13 | C. elegans | iglu#57 | C26 H26 O10 N3 P | 50.45347 78.95852 84.19692 86.60079 | 92.04975 94.02896 118.06521 |
| | | | C. | | | 93.81197 96.96928 107.59462 118.731 | 119.76913 120.04447 127.03911 |
| | | | briggsae | | | 135.08943 136.0407 155.07732 | 146.0239 180.06554 208.07593 |
| | | | | | | 159.37451 176.80008 179.85248 | 213.06586 226.08615 237.10211 |
| | | | | | | 203.34129 228.55795 251.19695 | 304.05392 339.0968 344.05188 |
| | | | | | | 340.05939 477.10785 570.12958 | 357.10742 455.08456 474.16568 |
| | | | | | | | 554.13129 572.14246 |
| 570.12776 | 572.14341 | 11.94 | C. elegans | iglu#58 | C26 H26 O10 N3 P | 66.03432 78.95855 86.44823 89.02406 | 92.04971 94.02893 118.06514 |
| | | | | | | 96.96931 101.02432 110.02501 | 120.04442 138.05501 154.04985 |
| | | | | | | 113.02461 119.0352 136.0407 | 204.06552 213.06586 220.06035 |
| | | | | | | 152.99615 197.99641 333.06076 | 225.60944 228.06528 304.05081 |
| | | | | | | 340.05981 358.07059 433.0817 | 318.03696 337.11792 339.09689 |
| | | | | | | 451.09204 477.10803 570.12976 | 344.05234 437.07437 455.0845 |
| | | | | | | 570.26953 | 474.16492 572.14246 |
| 571.11177 | 573.12742 | 11.10 | C. elegans | iglu#60 | C26 H25 O11 N2 P | 78.95856 94.46343 96.96933 | 85.06515 94.4136 105.03362 |
| | | | | | | 105.17922 107.94012 135.09593 | 105.19584 107.88454 107.88652 |
| | | | | | | 137.0247 155.08704 175.02075 | 110.54861 118.7561 124.46119 |
| | | | | | | 179.85925 210.36697 211.08948 | 135.11726 145.17136 171.05635 |
| | | | | | | 232.19424 340.05991 358.07065 | 183.45328 203.97406 283.10828 |
| | | | | | | 373.00793 451.09244 460.08267 | 301.1189 313.94199 379.54688 |
| | | | | | | 478.09177 571.11359 | 443.151 573.21356 |
| 463.09064 | 465.10629 | 8.30 | C. elegans | iglu#601 | C20H21N2O9P | 69.10169 78.95854 91.08374 91.08641 | 62.80962 78.0342 93.82679 |
| | | | C. | | | 93.81248 94.36591 96.96928 | 105.18945 106.02884 118.06516 |
| | | | briggsae | | | 105.17458 107.83212 118.73349 | 124.03935 135.10852 139.19931 |
| | | | | | | 122.02522 124.40034 152.99619 | 179.8812 184.07584 190.04988 |
| | | | | | | 180.99106 223.00215 227.83403 | 229.10727 244.09618 250.07076 |
| | | | | | | 266.02371 340.04138 340.05975 | 304.00165 304.05179 335.20093 |
| | | | | | | 463.09232 | 348.04742 465.10541 |
| 571.12300 | 573.13866 | 9.30 | C. elegans | iglu#62 | C25 H25 O10 N4 P | | 93.83233 94.02891 105.1953 |
| | | | | | | | 107.99771 109.76546 118.75726 |
| | | | | | | | 121.03963 122.56885 135.12115 |
| | | | | | | | 139.05034 163.62898 181.06062 |
| | | | | | | | 202.10742 223.09036 226.086 |
| | | | | | | | 304.05734 358.10266 456.08005 |
| | | | | | | | 475.16098 573.13654 |
| 580.13726 | 582.15291 | 13.45 | C. elegans | iglu#64 | C29 H28 O10 N P | 50.19467 74.61971 78.95853 86.59789 | |
| | | | | | | 86.60047 91.05507 93.81833 96.96585 | |
| | | | | | | 96.96928 118.7366 121.02959 | |
| | | | | | | 135.04587 203.34412 222.37341 | |
| | | | | | | 304.02606 322.05005 340.05991 | |
| | | | | | | 458.10199 462.09723 580.13934 | |
| 581.13251 | 583.14816 | 12.62 | C. elegans | iglu#65 | C28 H27 O10 N2 P | | 60.33326 92.04964 105.03355 |
| | | | C. | | | | 118.06509 120.04441 138.05499 |
| | | | briggsae | | | | 204.06541 208.07565 224.07063 |
| | | | | | | | 226.08624 228.06538 231.06506 |
| | | | | | | | 329.04205 330.1116 348.12286 |
| | | | | | | | 350.10168 448.07819 466.08939 |
| | | | | | | | 485.1701 583.14618 |
| 581.13251 | 583.14816 | 12.95 | C. elegans | iglu#66 | C28 H27 O10 N2 P | | 51.06701 61.41923 94.46964 |
| | | | C. | | | | 94.47563 94.47714 105.03346 |
| | | | briggsae | | | | 107.95544 107.95744 115.1044 |
| | | | | | | | 120.04446 124.54277 135.12593 |
| | | | | | | | 145.26636 166.05315 218.35295 |
| | | | | | | | 379.91803 440.84213 553.34918 |
| | | | | | | | 579.90594 602.37225 |
| 581.13251 | 583.14816 | 11.89 | C. elegans | iglu#68 | C28 H27 O10 N2 P | 50.14521 78.95863 92.05009 96.96935 | 52.69332 84.2081 93.8242 |
| | | | | | | 101.06067 118.73784 123.64352 | 105.07057 106.02841 114.8766 |
| | | | | | | 136.04083 148.96503 162.98083 | 118.74509 118.74798 120.04401 |
| | | | | | | 176.61932 203.16011 204.99126 | 127.08264 155.09833 159.16942 |
| | | | | | | 223.00058 225.76352 307.06 444.1084 | 160.60419 168.3136 184.28561 |
| | | | | | | 444.83072 452.81253 463.09344 | 223.17596 276.67178 304.05051 |
| | | | | | | | 311.69055 473.38074 |
| 582.12776 | 584.14341 | 9.81 | C. elegans | iglu#69 | C27 H26 O10 N3 P | | 50.12669 81.07024 84.08113 |
| | | | C. | | | | 86.06035 102.0914 105.19357 |
| | | | briggsae | | | | 115.03899 123.11692 159.06525 |
| | | | | | | | 168.13829 176.09177 202.10759 |
| | | | | | | | 216.12306 224.08234 224.08844 |
| | | | | | | | 242.1385 286.16479 304.05011 |
| | | | | | | | 369.23788 583.3584 |
| 582.12776 | 584.14341 | 9.93 | C. elegans | iglu#70 | C27H26N3O10P | 75.99512 78.95862 93.04609 93.82099 | |
| | | | C. | | | 94.8726 96.96951 103.13805 | |
| | | | briggsae | | | 105.18352 135.10265 137.0361 | |
| | | | | | | 155.09192 179.8692 224.6683 | |
| | | | | | | 224.68077 269.19095 340.06223 | |
| | | | | | | 460.09467 477.15143 533.1889 582.13 | |
| 595.14816 | 597.16381 | 13.16 | C. elegans | iglu#72 | C29 H29 O10 N2 P | 78.95603 78.9585 94.38822 94.71317 | 84.21089 94.47361 94.47594 |
| | | | C. | | | 96.96928 98.0412 105.17565 | 94.47756 107.9552 107.95763 |
| | | | briggsae | | | 107.85555 118.73389 124.42519 | 114.48551 118.74837 120.04446 |
| | | | | | | 136.04042 143.10783 155.08139 | 124.54308 124.54536 135.11223 |
| | | | | | | 177.65237 216.39723 222.43752 | 145.26634 155.10359 179.88391 |
| | | | | | | 340.05917 458.10233 477.11102 | 184.07358 204.06604 219.11618 |
| | | | | | | 595.14996 | 251.49884 425.87534 |
| 596.14341 | 598.15906 | 12.54 | C. elegans | iglu#74 | C28H28N3O10P | 78.95855 86.44032 86.44376 86.44765 | 94.51543 120.04448 179.89601 |
| | | | C. | | | 86.4519 92.05022 94.42886 96.96928 | 180.06555 208.07576 224.67764 |
| | | | briggsae | | | 96.97285 105.17121 107.89947 | 228.06544 237.10202 239.08134 |
| | | | | | | 118.72842 130.05424 136.0407 | 252.27156 275.08902 304.04739 |
| | | | | | | 195.16452 197.99658 322.04968 | 311.98508 344.05234 365.11307 |
| | | | | | | 459.09741 477.10645 596.14557 | 383.12341 425.31448 481.10013 |
| | | | | | | | 500.18063 598.15741 |
| 597.12742 | 599.14307 | 11.23 | C. elegans | iglu#75 | C28 H27 O11 N2 P | 78.95853 86.43831 86.44102 86.44529 | |
| | | | C. | | | 93.03419 94.39649 96.96928 | |
| | | | briggsae | | | 105.17153 124.4597 136.04076 | |
| | | | | | | 137.02469 186.43835 197.99597 | |
| | | | | | | 322.04874 340.05988 358.07144 | |
| | | | | | | 460.08145 477.10822 478.09238 | |
| | | | | | | 597.12952 | |
| 597.12742 | 599.14307 | 11.48 | C. elegans | iglu#76 | C28 H27 O11 N2 P | 78.95853 86.43831 86.44102 86.44529 | |
| | | | C. | | | 93.03419 94.39649 96.96928 | |
| | | | briggsae | | | 105.17153 124.4597 136.04076 | |
| | | | | | | 137.02469 186.43835 197.99597 | |
| | | | | | | 322.04874 340.05988 358.07144 | |
| | | | | | | 460.08145 477.10822 478.09238 | |
| | | | | | | 597.12952 | |
| 440.11104 | 442.12669 | 10.04 | C. elegans | iglu#801 | C19H24NO9P | 54.87051 66.23303 72.06934 78.95851 | 55.05485 83.04948 83.05086 |
| | | | C. | | | 86.60141 91.36636 94.42043 96.96927 | 85.02879 93.8312 105.19488 |
| | | | briggsae | | | 105.17832 107.89531 145.1844 | 118.06516 118.75545 127.03917 |
| | | | | | | 152.99634 203.33563 203.34517 | 135.11748 179.89085 184.07582 |
| | | | | | | 229.69711 237.62753 340.05972 | 224.5174 224.52698 225.01578 |
| | | | | | | 358.04483 358.07056 440.11249 | 226.08636 227.09138 244.09665 |
| | | | | | | | 304.05374 325.06735 |
| 597.13866 | 599.15431 | 9.78 | C. elegans | iglu#82 | C27 H27 O10 N4 P | | 92.04953 93.04471 120.04412 |
| | | | C. | | | | 121.03935 139.0499 181.06036 |
| | | | briggsae | | | | 223.14948 226.08565 237.10139 |
| | | | | | | | 240.076 246.07535 247.07045 |
| | | | | | | | 304.04962 345.04688 364.12839 |
| | | | | | | | 366.10666 384.1174 482.09427 |
| | | | | | | | 501.17523 599.15186 |
| 611.14307 | 613.15872 | 11.33 | C. elegans | iglu#86 | C29 H29 O11 N2 P | 64.12451 78.95865 86.611 96.96935 | 92.04948 93.82965 105.19279 |
| | | | C. | | | 100.42749 107.05019 123.62213 | 107.04888 120.04412 134.05965 |
| | | | briggsae | | | 124.12957 136.04097 151.04037 | 138.05464 154.06462 184.07539 |
| | | | | | | 197.99696 203.15912 223.71065 | 204.06526 226.08528 228.06477 |
| | | | | | | 322.05002 340.06042 377.87109 | 231.81743 254.08011 380.11145 |
| | | | | | | 474.09732 477.10876 492.10815 | 398.12146 478.08743 496.09885 |
| | | | | | | 611.14685 | 515.17963 613.15558 |
| 559.14816 | 561.16381 | 12.37 | C. elegans | iglu#88 | C26 H29 O10 N2 P | 50.74778 78.95854 86.60074 96.96588 | 55.05482 83.04945 92.04971 |
| | | | C. | | | 96.96929 99.04472 105.17433 | 118.06514 120.04443 138.05505 |
| | | | briggsae | | | 118.73063 136.04089 145.22742 | 202.0862 204.06544 209.0808 |
| | | | | | | 155.07918 157.74239 176.79813 | 224.93373 226.08618 228.06544 |
| | | | | | | 203.33965 322.04688 422.10147 | 237.10211 307.05725 326.13843 |
| | | | | | | 459.09723 477.10712 559.14984 | 328.11746 426.0947 444.10501 |
| | | | | | | 559.26013 | 463.18619 561.16248 |
| 581.13251 | 583.14816 | 11.70 | C. elegans | iglu#90 | C28 H27 O10 N2 P | 78.95863 80.9628 92.05024 96.96935 | 84.20647 86.37217 98.78072 |
| | | | C. | | | 98.97355 99.04488 100.04823 | 107.06625 118.74639 120.04408 |
| | | | briggsae | | | 120.96988 135.10355 136.04073 | 123.52393 135.10693 152.68658 |
| | | | | | | 137.0443 148.9649 149.96854 | 155.0974 204.06482 204.10146 |
| | | | | | | 150.96947 162.98128 204.9913 | 224.16904 224.1756 224.18076 |
| | | | | | | 305.04443 306.04758 307.04993 | 228.06505 304.04984 330.13248 |
| | | | | | | 444.09879 | 416.39114 446.11972 |
| 533.13251 | 535.14816 | 10.50 | C. elegans | iglu#92 | C24 H27 O10 N2 P | | 68.9276 105.18088 106.0285 |
| | | | C. | | | | 118.06483 118.73908 124.03893 |
| | | | briggsae | | | | 134.24907 135.09848 166.0493 |
| | | | | | | | 224.14023 224.14662 226.08511 |
| | | | | | | | 232.05936 250.06963 304.04288 |
| | | | | | | | 330.03583 409.5441 418.08829 |
| | | | | | | | 535.14594 535.22986 |
| 510.10260 | 512.11825 | 5.35 | C. elegans | mgglu#201 | C19 H22 O10 N5 P | 52.21332 52.533 78.95849 94.35941 | 94.47031 94.47292 94.4745 |
| | | | | | | 94.36308 96.96922 107.82236 | 105.20134 107.95188 107.95393 |
| | | | | | | 109.37122 118.7386 148.96425 | 110.62792 111.67295 118.76257 |
| | | | | | | 164.05835 217.90761 223.00165 | 124.53796 124.54041 135.125 |
| | | | | | | 224.87729 224.88983 295.5676 | 145.26309 155.11781 166.07242 |
| | | | | | | 304.01169 388.06735 388.0947 | 179.90004 236.35849 369.43243 |
| | | | | | | 510.1041 | 432.79636 512.17358 |
| 598.13390 | 600.14955 | 7.63 | C. elegans | mgglu#4 | C26 H26 O10 N5 P | | 76.00774 84.21896 93.83645 |
| | | | | | | | 94.5368 94.53891 102.0913 |
| | | | | | | | 105.03351 105.20074 108.02647 |
| | | | | | | | 108.02873 108.03069 118.76165 |
| | | | | | | | 121.02834 135.12436 145.36241 |
| | | | | | | | 150.07741 235.68721 445.41129 |
| | | | | | | | 600.14819 627.59656 |
| 616.14447 | 618.16012 | 8.12 | C. elegans | mgglu#6 | C26H28N5O11P | | 68.93729 76.00256 84.21359 |
| | | | C. | | | | 93.83085 94.53188 94.53376 |
| | | | briggsae | | | | 94.53529 105.03362 105.19475 |
| | | | | | | | 108.02131 108.02332 118.75581 |
| | | | | | | | 124.61809 124.62055 137.81123 |
| | | | | | | | 145.35622 155.10805 166.07239 |
| | | | | | | | 179.88976 583.98553 |
| 566.14274 | 568.15839 | 9.85 | C. elegans | nglu#10 | C25 H30 O12 N P | 50.53573 78.95863 96.96933 | |
| | | | C. | | | 122.02514 123.02855 136.04111 | |
| | | | briggsae | | | 148.96494 149.96852 162.98096 | |
| | | | | | | 223.00198 224.00566 328.0239 | |
| | | | | | | 329.02759 346.03485 347.03732 | |
| | | | | | | 429.07965 466.07541 484.08585 | |
| | | | | | | 498.10138 566.12836 | |
| 550.11144 | 552.12709 | 10.37 | C. elegans | nglu#3 | C24H26NO12P | 50.97542 78.9586 86.61049 86.61312 | 75.99463 83.04922 84.20476 |
| | | | C. | | | 96.96613 96.96935 121.02977 | 93.75755 93.82088 105.03326 |
| | | | briggsae | | | 122.02511 136.10292 148.96513 | 105.18349 106.02848 107.14002 |
| | | | | | | 162.98067 188.1601 203.16214 | 118.74063 118.7428 124.03899 |
| | | | | | | 223.0025 223.35406 328.02423 | 129.09413 211.09779 251.22444 |
| | | | | | | 346.03537 428.07663 482.08896 | 330.03616 354.095 430.08789 |
| | | | | | | 550.1131 | 450.89221 452.07233 |
| 550.11144 | 552.12709 | 10.60 | C. elegans | nglu#4 | C24H26NO12P | 50.97542 78.9586 86.61049 86.61312 | 75.99463 83.04922 84.20476 |
| | | | C. | | | 96.96613 96.96935 121.02977 | 93.75755 93.82088 105.03326 |
| | | | briggsae | | | 122.02511 136.10292 148.96513 | 105.18349 106.02848 107.14002 |
| | | | | | | 162.98067 188.1601 203.16214 | 118.74063 118.7428 124.03899 |
| | | | | | | 223.0025 223.35406 328.02423 | 129.09413 211.09779 251.22444 |
| | | | | | | 346.03537 428.07663 482.08896 | 330.03616 354.095 430.08789 |
| | | | | | | 550.1131 | 450.89221 452.07233 |
| 551.10669 | 553.12234 | 9.39 | C. elegans | nglu#5 | C23 H25 O12 N2 P | 55.65717 55.65928 55.6613 66.05759 | |
| | | | C. | | | 76.68676 84.1953 92.0252 93.80959 | |
| | | | briggsae | | | 94.40538 124.46951 135.03159 | |
| | | | | | | 145.15668 168.74719 170.83258 | |
| | | | | | | 171.69708 191.05792 267.07428 | |
| | | | | | | 301.16583 327.33124 551.23724 | |
| 551.10669 | 553.12234 | 9.09 | C. elegans | nglu#6 | C23 H25 O12 N2 P | | 78.03427 93.82999 94.51088 |
| | | | C. | | | | 94.51252 105.1928 106.02884 |
| | | | briggsae | | | | 107.99715 118.75442 124.03938 |
| | | | | | | | 135.11517 145.321 155.10585 |
| | | | | | | | 179.88866 211.11786 232.05994 |
| | | | | | | | 304.0123 330.03714 355.0921 |
| | | | | | | | 453.06909 553.12244 |
| 565.12234 | 567.13799 | 9.80 | C. elegans | nglu#7 | C24H27N2O12P | 78.03448 78.95865 86.61113 96.96938 | |
| | | | C. | | | 99.04488 122.02518 136.04086 | |
| | | | briggsae | | | 148.96506 162.98065 203.15594 | |
| | | | | | | 204.99132 223.00203 328.02396 | |
| | | | | | | 346.03452 410.06723 428.07791 | |
| | | | | | | 465.07178 483.08252 497.09793 | |
| | | | | | | 565.12531 | |
| 565.12234 | 567.13799 | 9.89 | C. elegans | nglu#8 | C24H27N2O12P | 50.56276 78.95865 86.6122 93.79964 | |
| | | | | | | 96.9694 122.0252 135.10483 | |
| | | | | | | 136.04079 148.96495 162.98065 | |
| | | | | | | 176.61952 203.16058 204.99161 | |
| | | | | | | 223.00191 223.97018 328.02408 | |
| | | | | | | 346.03482 428.07697 497.05615 | |
| | | | | | | 565.12537 | |
| 565.12234 | 567.13799 | 10.24 | C. elegans | nglu#9 | C24H27N2O12P | 50.56326 78.95863 89.91605 96.96941 | |
| | | | C. | | | 99.04501 105.18342 122.02541 | |
| | | | briggsae | | | 123.59947 123.6055 136.04094 | |
| | | | | | | 144.16789 148.96498 179.86871 | |
| | | | | | | 204.3224 229.31738 305.04449 | |
| | | | | | | 433.6268 442.09259 465.40302 | |
| | | | | | | 565.12524 | |
| 595.16929 | 597.18494 | 7.48 | C. elegans | oglu#10 | C26 H33 O12 N2 P | 75.98877 78.9585 86.4398 86.44263 | |
| | | | C. | | | 86.44572 86.4501 96.96925 99.04483 | |
| | | | briggsae | | | 107.89776 118.73374 136.04076 | |
| | | | | | | 148.96519 152.07201 155.08113 | |
| | | | | | | 195.3494 197.9971 305.04462 | |
| | | | | | | 458.12308 476.1344 595.17108 | |
| 606.14889 | 608.16454 | 6.89 | C. elegans | oglu#1401 | C26 H30 O12 N3 P | | 78.95863 79.96284 80.96288 |
| | | | C. | | | | 86.61456 92.05025 96.96936 |
| | | | briggsae | | | | 98.97356 122.02514 123.02853 |
| | | | | | | | 124.02956 136.04089 137.04451 |
| | | | | | | | 148.96512 193.67819 193.68692 |
| | | | | | | | 203.15147 465.10831 466.11197 |
| | | | | | | | 467.11343 604.16223 |
| 618.14889 | 620.16454 | 7.76 | C. elegans | oglu#15 | C27 H30 O12 N3 P | 78.95861 86.61366 96.96935 | 81.03355 92.04937 105.18671 |
| | | | C. | | | 107.09937 122.02507 136.04073 | 106.0285 109.02814 118.74568 |
| | | | briggsae | | | 148.96466 151.85791 151.86357 | 120.04408 124.03898 137.05927 |
| | | | | | | 152.07219 197.99693 203.14453 | 155.09991 166.04944 225.06477 |
| | | | | | | 203.15437 227.53514 328.02426 | 228.0647 230.4268 250.06999 |
| | | | | | | 358.07053 481.10306 499.11383 | 330.03571 369.10638 467.08328 |
| | | | | | | 520.05133 618.15253 | 522.18536 620.16064 |
| 618.14889 | 620.16454 | 6.48 | C. elegans | oglu#16 | C27 H30 O12 N3 P | 78.03455 78.95862 83.23592 86.60986 | 81.03353 92.04942 106.02847 |
| | | | | | | 86.61299 92.05029 96.96933 107.0701 | 109.02811 120.04404 124.03899 |
| | | | | | | 122.02512 136.04082 148.96526 | 134.05972 138.05452 166.04936 |
| | | | | | | 152.07289 197.99696 203.15376 | 210.05417 225.06512 228.06477 |
| | | | | | | 204.99153 224.29291 328.02383 | 238.08557 330.0358 369.10672 |
| | | | | | | 481.10318 499.11426 618.15253 | 467.08322 504.17471 522.185 |
| | | | | | | | 602.15106 620.16077 |
| 633.14855 | 635.16420 | 7.00 | C. elegans | oglu#17 | C28 H31 O13 N2 P | 78.95853 86.60219 94.41171 96.96928 | 62.98522 97.0331 109.89562 |
| | | | | | | 107.88508 149.17809 158.34164 | 118.74021 118.74279 122.51504 |
| | | | | | | 168.35939 177.26093 178.16046 | 126.52717 135.10225 155.09164 |
| | | | | | | 242.67334 311.11865 323.10303 | 156.39464 166.08394 287.88834 |
| | | | | | | 355.07425 357.08954 432.10312 | 304.04697 304.05743 310.75339 |
| | | | | | | 503.1423 633.20514 638.94067 | 314.88446 371.62469 421.25693 |
| | | | | | | 654.26074 | 492.90884 635.13025 |
| 653.21115 | 655.22680 | 8.12 | C. elegans | oglu#22 | C29 H39 O13 N2 P | 61.18382 64.59567 78.95853 94.37922 | 81.07 93.82537 99.08028 99.08198 |
| | | | C. | | | 96.96917 105.18045 107.84237 | 107.11494 120.04404 127.03868 |
| | | | briggsae | | | 107.84714 110.51813 124.41151 | 136.07539 201.11176 204.065 |
| | | | | | | 137.67148 157.08772 179.86691 | 228.06473 228.94031 246.07521 |
| | | | | | | 182.35612 238.97339 516.16705 | 260.12674 304.04816 386.15817 |
| | | | | | | 534.16901 535.32544 638.93719 | 404.16913 454.40298 502.14575 |
| | | | | | | 653.21387 | 655.2229 |
| 603.13799 | 605.15364 | 6.86 | C. | oglu#24 | C27 H29 O12 N2 P | | 68.92249 93.81215 105.03325 |
| | | | briggsae | | | | 105.17229 106.02848 109.59373 |
| | | | | | | | 124.03896 135.08922 166.33424 |
| | | | | | | | 211.07649 226.80821 304.04407 |
| | | | | | | | 304.05222 330.03552 354.0954 |
| | | | | | | | 452.07257 516.68304 587.13989 |
| | | | | | | | 603.08386 605.15192 |
| 580.13324 | 582.14889 | 6.68 | C. elegans | oglu#26 | C24 H28 O12 N3 P | | 68.93159 75.99727 93.82426 |
| | | | | | | | 94.02731 94.02891 94.53119 |
| | | | | | | | 94.53275 105.18555 105.1875 |
| | | | | | | | 108.01796 108.02004 124.61395 |
| | | | | | | | 124.61648 145.3511 155.09741 |
| | | | | | | | 179.87709 251.64618 331.09158 |
| | | | | | | | 429.06866 582.14899 |
| 512.11960 | 514.13525 | 5.88 | C. | oglu#4 | C21 H26 N2 O11 P | | 64.00549 68.93542 76.00138 |
| | | | briggsae | | | | 84.21204 93.82886 94.4688 |
| | | | | | | | 94.47048 105.19212 107.94907 |
| | | | | | | | 107.95111 118.75205 120.04439 |
| | | | | | | | 124.53519 135.11348 145.25681 |
| | | | | | | | 155.10475 179.88495 195.68289 |
| | | | | | | | 362.06335 385.87286 |
| 512.11960 | 514.13525 | 6.11 | C. | oglu#401 | C21 H26 N2 O11 P | | 64.00549 68.93542 76.00138 |
| | | | briggsae | | | | 84.21204 93.82886 94.4688 |
| | | | | | | | 94.47048 105.19212 107.94907 |
| | | | | | | | 107.95111 118.75205 120.04439 |
| | | | | | | | 124.53519 135.11348 145.25681 |
| | | | | | | | 155.10475 179.88495 195.68289 |
| | | | | | | | 362.06335 385.87286 |
| 569.11725 | 571.13290 | 7.21 | C. elegans | oglu#601 | C23 H27 O13 N2 P | | 55.05483 57.47219 63.64296 |
| | | | | | | | 83.04945 94.02894 105.19753 |
| | | | | | | | 109.02843 118.75926 136.07581 |
| | | | | | | | 155.11441 168.16273 220.06064 |
| | | | | | | | 223.40176 223.4086 227.09103 |
| | | | | | | | 304.05652 320.11234 336.26828 |
| | | | | | | | 418.08923 571.16809 |
| 591.13799 | 593.15364 | 7.92 | C. elegans | oglu#7 | C26 H29 O12 N2 P | 75.98412 78.95849 86.59887 88.80653 | 94.02895 105.03361 105.19658 |
| | | | | | | 94.36419 96.96941 107.82886 | 109.02847 127.03912 136.07576 |
| | | | | | | 107.83305 110.02536 118.72643 | 158.14162 178.04982 220.06035 |
| | | | | | | 135.08273 145.09302 225.10747 | 222.38564 222.39233 231.06526 |
| | | | | | | 254.60886 302.97214 360.08807 | 249.07584 329.04156 342.09656 |
| | | | | | | 361.09085 454.11169 591.15948 | 440.07373 495.177 575.1424 |
| | | | | | | 591.20917 | 593.15247 593.17804 |
| 591.13799 | 593.15364 | 7.51 | C. elegans | oglu#8 | C26 H29 O12 N2 P | 66.90678 67.27675 72.82223 78.95847 | |
| | | | | | | 87.00389 96.96919 107.88424 | |
| | | | | | | 118.73598 121.02967 135.18114 | |
| | | | | | | 145.16844 155.08122 209.80818 | |
| | | | | | | 251.2094 319.06378 319.42209 | |
| | | | | | | 460.32184 498.11823 502.74203 | |
| | | | | | | 591.13971 | |
| 595.16929 | 597.18494 | 7.80 | C. elegans | oglu#9 | C26 H33 O12 N2 P | 53.9586 78.95852 86.44103 86.44378 | |
| | | | | | | 86.44806 86.45277 94.38497 96.96928 | |
| | | | | | | 99.04478 107.85302 118.73399 | |
| | | | | | | 136.04082 152.0719 197.99664 | |
| | | | | | | 204.9915 305.04425 358.07086 | |
| | | | | | | 458.12354 476.13516 595.17139 | |
| 456.06957 | 458.08522 | 8.77 | C. elegans | pyglu#201 | C18 H20 O11 N P | 54.19283 67.14862 78.95849 86.60259 | |
| | | | C. | | | 96.96925 105.1713 108.28411 | |
| | | | briggsae | | | 110.02509 118.72769 121.02974 | |
| | | | | | | 148.96481 156.38882 200.99669 | |
| | | | | | | 223.00179 229.55624 241.01233 | |
| | | | | | | 315.25778 334.0343 345.03873 | |
| | | | | | | 456.07101 | |
| 538.11144 | 540.12709 | 11.22 | C. elegans | pyglu#4 | C23 H26 O12 N P | 78.95852 93.8125 96.96925 96.97246 | |
| | | | C. | | | 99.0445 102.92152 105.1747 | |
| | | | briggsae | | | 107.94077 107.9452 124.52478 | |
| | | | | | | 124.52991 148.96484 155.07573 | |
| | | | | | | 228.34386 305.04428 323.05463 | |
| | | | | | | 416.07645 515.54144 538.11346 | |
| | | | | | | 538.16016 | |
| 755.27923 | 757.29488 | 7.95 | C. elegans | tyglas#1 | C34 H49 O15 N2 P | | 60.24321 60.24407 81.03378 |
| | | | | | | | 83.08586 94.54935 95.04935 |
| | | | | | | | 108.03953 108.04158 111.08059 |
| | | | | | | | 120.04446 121.0648 127.03902 |
| | | | | | | | 138.05511 138.0914 225.01572 |
| | | | | | | | 228.06543 257.12805 627.22992 |
| | | | | | | | 721.27112 757.2912 |
| 713.23228 | 715.24793 | 7.23 | C. elegans | tyglas#11 | C31 H43 O15 N2 P | 53.59522 68.92701 73.0289 78.95861 | 93.7797 95.04913 105.18098 |
| | | | C. | | | 96.96938 105.17933 123.58164 | 118.74019 119.75354 120.04411 |
| | | | briggsae | | | 177.66785 179.862 203.14912 | 144.19832 155.09038 179.86526 |
| | | | | | | 227.49893 392.33493 475.85114 | 211.09317 223.20984 304.05075 |
| | | | | | | 576.18774 635.1568 640.97382 | 543.85431 585.18225 640.99481 |
| | | | | | | 659.52533 713.23694 719.05774 | 641.02032 715.24432 718.23761 |
| | | | | | | 719.13 | 718.38623 718.41931 |
| 699.21663 | 701.23228 | 7.03 | C. | ty glas#5 | C30 H41 O15 N2 P | 65.81332 73.02895 78.95863 86.61209 | 61.89407 89.25365 93.76373 |
| | | | briggsae | | | 93.75508 96.96943 105.17838 | 95.04896 107.14086 118.74176 |
| | | | | | | 122.78951 123.59331 123.5981 | 120.04406 138.05463 179.86647 |
| | | | | | | 203.15404 224.60686 251.21062 | 228.06499 228.59018 228.597 |
| | | | | | | 414.09793 562.1731 640.98218 | 257.12762 294.82532 308.49683 |
| | | | | | | 655.69714 699.22131 719.06665 | 486.31381 571.16589 573.71655 |
| | | | | | | 719.13306 | 701.22559 718.54132 |
| 753.26358 | 755.27923 | 7.89 | C. elegans | tyglas#7 | C34 H47 O15 N2 P | 60.24379 60.24461 81.07024 95.04939 | |
| | | | | | | 107.82131 109.06489 120.04448 | |
| | | | | | | 121.06483 127.07542 138.05508 | |
| | | | | | | 138.09148 179.89413 204.06531 | |
| | | | | | | 228.06537 228.10193 246.11206 | |
| | | | | | | 257.12817 390.15378 625.21436 | |
| | | | | | | 755.2771 | |
| 608.21082 | 610.22647 | 5.95 | C. | tyglas#9 | C25 H40 O14 N P | | 75.99136 93.817 95.04901 |
| | | | briggsae | | | | 97.02812 98.98394 107.11195 |
| | | | | | | | 118.73573 121.06428 138.09094 |
| | | | | | | | 155.08435 179.23259 179.86174 |
| | | | | | | | 227.00032 227.00664 304.05103 |
| | | | | | | | 306.81616 480.16034 502.05844 |
| | | | | | | | 542.46747 610.22247 |
| 615.17437 | 617.19002 | 8.63 | C. elegans | tyglu#12 | C29 H33 O11 N2 P | 75.98882 78.95864 86.61316 91.05504 | 60.33598 81.03383 91.05449 |
| | | | C. | | | 96.96937 135.04588 136.041 | 105.19788 109.02847 119.76976 |
| | | | briggsae | | | 148.96495 191.72887 197.99738 | 120.04444 121.06487 138.05482 |
| | | | | | | 203.15289 223.00259 229.16876 | 204.06541 225.01595 228.06561 |
| | | | | | | 341.04318 360.08655 378.09814 | 229.81612 238.08591 257.12805 |
| | | | | | | 478.12891 496.13943 497.13458 | 480.10492 519.21301 617.15936 |
| | | | | | | 615.17822 | 617.18787 640.1665 |
| 462.15291 | 464.16856 | 6.58 | C. | tyglu#131 | C19 H30 NO 10 P | 51.79475 53.92133 65.89232 75.99543 | 57.07029 69.03375 81.03364 |
| | | | briggsae | | | 78.95863 93.68916 93.82409 94.28223 | 85.06483 85.06618 93.77364 |
| | | | | | | 96.96954 105.18489 121.27247 | 97.02823 98.98402 105.17441 |
| | | | | | | 179.86879 179.87704 225.79791 | 118.73328 121.06445 127.03858 |
| | | | | | | 250.76633 257.86258 360.08722 | 138.09096 155.08115 225.01508 |
| | | | | | | 378.09723 388.23373 462.15512 | 229.10617 243.02557 366.18954 |
| | | | | | | | 464.15121 464.16644 |
| 581.19002 | 583.20567 | 8.37 | C. elegans | tyglu#14 | C26 H35 O11 N2 P | 50.1934 73.39599 78.95853 86.6016 | |
| | | | C. | | | 86.60457 96.96928 99.04477 | |
| | | | briggsae | | | 100.04816 135.10048 136.04063 | |
| | | | | | | 137.04398 148.96498 164.05795 | |
| | | | | | | 203.3414 223.43813 361.08994 | |
| | | | | | | 442.12766 443.13147 461.14218 | |
| | | | | | | 580.17938 | |
| 601.15927 | 603.17495 | 7.97 | C. elegans | tyglu#16 | C28H31N2O11P | Previously reported (Le et. al. elife | Previously reported (Le et. al. elife |
| | | | C. | | | 2020) | 2020) |
| | | | briggsae | | | | |
| 471.11686 | 473.13251 | 5.92 | C. elegans | tyglu#181 | C19 H25 O10 N2 P | | 81.03386 94.02896 94.51291 |
| | | | C. | | | | 94.51451 98.98432 112.0393 |
| | | | briggsae | | | | 121.06483 126.06625 127.03906 |
| | | | | | | | 134.08121 138.0914 178.03598 |
| | | | | | | | 225.01553 231.11276 238.07094 |
| | | | | | | | 311.10947 336.04733 375.15439 |
| | | | | | | | 473.10684 473.13141 |
| 553.15872 | 555.17437 | 7.98 | C. elegans | tyglu#19 | C24 H31 O11 N2 P | 78.95852 81.14077 83.48387 96.9695 | 55.05487 81.03388 83.0495 |
| | | | | | | 107.90952 124.48722 135.08231 | 94.02898 97.02859 109.02842 |
| | | | | | | 182.23465 185.71225 227.69679 | 121.0649 122.06005 127.03907 |
| | | | | | | 227.70854 281.81662 337.78198 | 138.09149 209.08075 220.06036 |
| | | | | | | 365.42944 379.56628 387.06747 | 227.09131 231.11285 304.0556 |
| | | | | | | 407.10092 460.13895 466.36899 | 307.05722 320.1124 418.0896 |
| | | | | | | 553.16083 | 457.1973 555.17303 |
| 497.13251 | 499.14816 | 6.27 | C. elegans | tyglu#2 | C21H27N2O10P | 52.6246 52.62581 75.60188 78.95864 | 75.99859 88.57979 105.18658 |
| | | | | | | 86.61191 89.4277 91.38159 96.96936 | 107.05492 120.04404 121.06434 |
| | | | | | | 105.17608 136.04103 148.96487 | 138.05447 138.091 214.63907 |
| | | | | | | 162.98059 197.99684 203.1485 | 221.90469 227.01039 227.01979 |
| | | | | | | 218.26247 223.00189 229.69888 | 257.12741 264.08533 278.40604 |
| | | | | | | 360.08649 378.09756 497.13431 | 304.04785 362.06183 401.16895 |
| | | | | | | | 498.31732 499.14563 |
| 553.15872 | 555.17437 | 7.46 | C. elegans | tyglu#20 | C24 H31 O11 N2 P | 73.02894 78.9586 86.61082 96.96611 | 81.03369 109.02816 120.04415 |
| | | | C. | | | 96.96934 107.07524 118.74115 | 121.06458 138.05466 138.09109 |
| | | | briggsae | | | 128.01768 136.04065 136.07692 | 147.05833 176.07005 204.06502 |
| | | | | | | 146.08275 203.15405 217.11967 | 226.13992 228.06503 257.12753 |
| | | | | | | 275.10785 277.12363 342.07599 | 295.12491 302.10129 304.04938 |
| | | | | | | 416.11246 434.12427 553.16034 | 320.11185 418.08835 425.18768 |
| | | | | | | 553.23077 | 457.19553 555.17188 |
| 555.17437 | 557.19002 | 8.13 | C. elegans | tyglu#22 | C24 H33 O11 N2 P | | 57.07045 75.99844 81.03386 |
| | | | C. | | | | 84.2086 85.06508 94.02891 |
| | | | briggsae | | | | 94.53815 94.53977 105.18771 |
| | | | | | | | 108.0273 108.02937 121.06485 |
| | | | | | | | 138.09164 202.10715 225.0152 |
| | | | | | | | 322.12796 420.10483 459.21265 |
| | | | | | | | 557.18829 557.34167 |
| 564.13832 | 566.15397 | 7.42 | C. elegans | tyglu#24 | C24 H28 O11 N3 P | | 81.03385 94.02895 94.60809 |
| | | | | | | | 109.02845 112.0393 118.7502 |
| | | | | | | | 121.0648 122.06017 127.03908 |
| | | | | | | | 135.11229 138.09143 158.13832 |
| | | | | | | | 179.88203 220.06018 223.06908 |
| | | | | | | | 238.071 318.0369 331.09183 |
| | | | | | | | 429.06873 468.17575 |
| 564.16347 | 566.17912 | 8.38 | C. | tyglu#26 | C26 H32 O11 N P | | 55.05481 66.60278 81.03384 |
| | | | briggsae | | | | 83.04942 84.20998 94.50158 |
| | | | | | | | 105.03352 105.18992 107.98544 |
| | | | | | | | 107.98739 109.02841 118.75079 |
| | | | | | | | 121.06474 124.57764 135.1111 |
| | | | | | | | 138.09138 145.30716 331.117 |
| | | | | | | | 468.20068 566.17743 |
| 575.14307 | 577.15872 | 8.17 | C. elegans | tyglu#28 | C26 H29 O11 N2 P | 50.32233 73.16942 78.95853 84.1999 | 53.03919 81.03381 94.02897 |
| | | | C. | | | 85.5389 86.60282 96.96928 110.02496 | 105.03362 105.19012 109.02843 |
| | | | briggsae | | | 118.73595 121.02972 155.08179 | 121.06483 122.06001 127.03912 |
| | | | | | | 199.25941 199.26868 203.3382 | 138.0914 155.10275 178.05002 |
| | | | | | | 203.34911 345.03961 360.08627 | 220.06027 225.42741 231.06488 |
| | | | | | | 464.11279 482.12317 575.14496 | 231.11259 342.09659 440.07401 |
| | | | | | | | 479.18115 577.15747 |
| 576.13832 | 578.15397 | 6.88 | C. elegans | tyglu#30 | C25 H28 O11 N3 P | | 81.03348 84.30078 94.02866 |
| | | | C. | | | | 105.17885 106.02839 109.02811 |
| | | | briggsae | | | | 124.03883 135.0986 137.74377 |
| | | | | | | | 155.08699 179.86372 224.75401 |
| | | | | | | | 250.06946 304.04413 304.05258 |
| | | | | | | | 330.03583 343.09061 480.17462 |
| | | | | | | | 517.28137 578.151 |
| 586.14782 | 588.16347 | 8.61 | C. elegans | tyglu#32 | C28 H30 O11 N P | | 53.03908 54.96523 66.70992 |
| | | | C. | | | | 66.71709 66.71796 81.03362 |
| | | | briggsae | | | | 105.0333 109.02811 118.74069 |
| | | | | | | | 121.06452 138.01875 138.091 |
| | | | | | | | 155.09171 165.05415 204.1011 |
| | | | | | | | 251.22211 329.04111 353.10019 |
| | | | | | | | 490.18478 588.16095 |
| 590.15397 | 592.16962 | 7.70 | C. elegans | tyglu#34 | C26 H30 O11 N3 P | 75.99413 78.95851 86.44268 94.40981 | |
| | | | C. | | | 96.96594 96.96926 102.19826 | |
| | | | briggsae | | | 105.18217 107.88169 110.02503 | |
| | | | | | | 153.28856 155.08896 186.84142 | |
| | | | | | | 205.85085 244.58777 317.42334 | |
| | | | | | | 360.08618 453.10837 497.134 | |
| | | | | | | 590.15588 | |
| 617.15364 | 619.16929 | 8.17 | C. elegans | tyglu#35 | C28 H31 O12 N2 P | 61.17215 61.55349 75.98811 78.95852 | 93.72557 105.03333 105.19283 |
| | | | C. | | | 86.4497 94.38222 96.96928 97.63271 | 107.10244 120.04408 144.11087 |
| | | | briggsae | | | 100.1926 105.17321 107.84954 | 154.08571 155.10246 156.59241 |
| | | | | | | 121.02962 143.64838 152.07216 | 179.88347 220.44116 224.06985 |
| | | | | | | 197.99702 279.33984 358.07059 | 230.42445 273.12274 274.22974 |
| | | | | | | 480.10822 498.11969 617.15552 | 521.19025 534.9801 619.13995 |
| | | | | | | | 619.16602 641.64764 |
| 617.15364 | 619.16929 | 7.64 | C. elegans | tyglu#36 | C28 H31 O12 N2 P | 78.9585 86.60172 86.60445 94.46476 | 60.33445 60.3374 81.03386 |
| | | | | | | 96.96925 105.17616 124.52258 | 92.04973 105.03362 109.02854 |
| | | | | | | 124.52807 136.04048 137.0246 | 119.76378 119.76597 120.04448 |
| | | | | | | 203.34566 223.00179 360.08652 | 134.06015 136.07578 138.05513 |
| | | | | | | 401.56122 480.10727 497.13373 | 165.05476 204.06561 224.07057 |
| | | | | | | 498.11914 617.1554 638.90851 | 228.06551 350.1019 466.08942 |
| | | | | | | 638.96381 | 521.19147 619.1676 |
| 631.16929 | 633.18494 | 7.81 | C. elegans | tyglu#37 | C29 H33 O12 N2 P | 78.95863 86.44883 86.45142 86.45596 | 60.34156 60.34229 81.03358 |
| | | | C. | | | 96.96938 105.18013 107.05057 | 92.04944 97.02827 107.04884 |
| | | | briggsae | | | 123.53516 136.04099 151.04063 | 109.02806 120.04406 121.06441 |
| | | | | | | 189.02975 197.9973 203.02156 | 122.05967 127.0386 138.05467 |
| | | | | | | 223.00204 360.08682 494.12369 | 204.06487 228.06485 254.08017 |
| | | | | | | 496.92044 497.1344 512.13574 | 257.12741 398.12161 496.09909 |
| | | | | | | 631.17316 | 535.20593 633.18109 |
| 617.16487 | 619.18052 | 6.28 | C. elegans | tyglu#38 | C27 H31 O11 N4 P | | 93.71703 97.1646 105.96069 |
| | | | C. | | | | 107.08604 115.19736 118.74855 |
| | | | briggsae | | | | 120.04401 121.03928 177.62076 |
| | | | | | | | 181.06004 205.80489 225.58636 |
| | | | | | | | 230.03525 240.0759 242.93527 |
| | | | | | | | 346.34448 423.45682 442.78842 |
| | | | | | | | 482.09479 619.17676 |
| 616.17017 | 618.18585 | 7.65 | C. elegans | tyglu#4 | C28H32N3O11P | Previously reported (Le et. al. elife | Previously reported (Le et. al. elife |
| | | | C. | | | 2020) | 2020) |
| | | | briggsae | | | | |
| 644.20092 | 646.21657 | 7.00 | C. elegans | tyglu#42 | C30 H36 O11 N3 P | 78.95862 86.61346 92.05039 96.96934 | 81.03355 109.02808 120.04406 |
| | | | C. | | | 136.04094 147.04562 148.96515 | 120.08043 121.06438 138.05429 |
| | | | briggsae | | | 164.07227 186.94196 197.99725 | 166.08563 174.05446 208.09619 |
| | | | | | | 203.15608 225.0878 225.09981 | 225.68857 228.06477 229.08514 |
| | | | | | | 342.07553 360.08643 370.07114 | 246.07489 246.11154 347.13748 |
| | | | | | | 505.29373 507.15512 525.16632 | 393.14267 411.15366 509.13025 |
| | | | | | | 644.20459 | 548.23804 646.21283 |
| 653.21115 | 655.22680 | 8.20 | C. elegans | tyglu#45 | C29 H39 O13 N2 P | 61.18382 64.59567 78.95853 94.37922 | 81.06992 99.08027 105.18147 |
| | | | C. | | | 96.96917 105.18045 107.84237 | 118.74126 120.04407 127.03879 |
| | | | briggsae | | | 107.84714 110.51813 124.41151 | 159.10115 201.11153 204.0649 |
| | | | | | | 137.67148 157.08772 179.86691 | 210.05428 223.23506 228.06485 |
| | | | | | | 182.35612 238.97339 516.16705 | 260.12717 304.04736 404.16827 |
| | | | | | | 534.16901 535.32544 638.93719 | 484.13531 502.14621 557.24731 |
| | | | | | | 653.21387 | 640.99615 655.22278 |
| 645.22132 | 647.23697 | 10.32 | C. elegans | tyglu#50 | C31 H39 O11 N2 P | | 62.06038 79.05441 92.04946 |
| | | | C. | | | | 93.06983 105.17999 106.0285 |
| | | | briggsae | | | | 107.08528 120.04161 120.04408 |
| | | | | | | | 121.06445 124.03898 149.09567 |
| | | | | | | | 225.84108 250.07001 268.13214 |
| | | | | | | | 304.04843 348.04596 640.96515 |
| | | | | | | | 647.23401 647.29163 |
| 483.11686 | 485.13251 | 5.38 | C. | tyglu#501 | C20 H25 O10 N2 P | 53.12169 78.95864 80.69837 86.61335 | 75.99845 93.80305 93.80476 |
| | | | briggsae | | | 91.91232 94.08073 96.96934 | 93.82536 97.0284 105.18829 |
| | | | | | | 115.05273 135.10179 135.55511 | 106.02856 107.18826 109.02825 |
| | | | | | | 223.00189 228.7077 251.21904 | 118.74774 121.06449 124.03904 |
| | | | | | | 292.12033 297.29691 360.08701 | 127.03864 135.10851 138.09093 |
| | | | | | | 410.54266 424.12723 437.41629 | 144.23169 179.8786 250.07005 |
| | | | | | | 483.1189 | 348.04633 485.13043 |
| 567.17437 | 569.19002 | 7.90 | C. elegans | tyglu#52 | C25 H33 O11 N2 P | 78.95863 85.9894 86.61401 86.61683 | 60.34161 71.04934 81.03354 |
| | | | C. | | | 87.04481 96.96934 136.04054 | 92.04939 97.02821 109.02812 |
| | | | briggsae | | | 136.07735 148.96494 197.99696 | 120.04404 121.06442 138.05452 |
| | | | | | | 200.9211 203.1541 204.99136 | 138.09103 190.08574 204.06497 |
| | | | | | | 225.01137 225.02298 293.04437 | 225.01505 228.06471 257.12744 |
| | | | | | | 342.07599 430.12863 448.13889 | 316.11658 334.12692 432.10382 |
| | | | | | | 567.17712 | 471.21072 569.18701 |
| 560.22607 | 562.24172 | 7.75 | C. | tyglu#53 | C25 H40 O11 N P | 50.69523 78.95863 86.61288 93.81935 | 69.03374 81.06994 83.08551 |
| | | | briggsae | | | 96.96925 105.18023 179.86255 | 85.0648 97.02825 98.98399 |
| | | | | | | 203.15367 209.76151 223.00206 | 105.0696 121.06442 123.11643 |
| | | | | | | 223.7849 251.21571 258.62549 | 127.03858 138.0909 147.11633 |
| | | | | | | 340.66168 356.53552 360.08688 | 165.12689 183.13745 223.80045 |
| | | | | | | 378.09717 534.94446 552.56836 | 223.80785 304.05106 327.17883 |
| | | | | | | 560.22888 | 562.23853 562.28607 |
| 544.23116 | 546.24681 | 11.07 | C. | tyglu#54 | C25H40NO10P | 51.14393 51.14526 71.68391 78.95865 | 69.0337 69.0701 83.08562 |
| | | | briggsae | | | 86.61308 86.61581 96.96938 | 93.06974 97.02821 98.98404 |
| | | | | | | 144.72957 155.078 179.85292 | 107.08522 111.08008 121.0644 |
| | | | | | | 192.0585 203.16914 223.00226 | 127.03853 135.0965 138.09093 |
| | | | | | | 223.1116 223.125 241.01273 | 149.13205 167.14258 224.04318 |
| | | | | | | 290.68979 360.08685 378.09726 | 224.05028 225.01485 243.02554 |
| | | | | | | 383.42645 | 311.18408 448.26761 |
| 649.25262 | 651.26827 | 10.26 | C. | tyglu#56 | C31 H43 O11 N2 P | 59.24969 68.93102 78.95866 86.60941 | 69.07012 81.03357 83.08556 |
| | | | briggsae | | | 86.61276 96.9694 122.02523 | 93.06975 106.02847 109.02813 |
| | | | | | | 176.61867 203.16077 223.00317 | 121.06439 122.05968 124.03896 |
| | | | | | | 223.70306 226.54718 227.20074 | 127.03851 149.13194 166.04942 |
| | | | | | | 360.0871 465.10904 483.08249 | 187.07469 250.06998 330.03595 |
| | | | | | | 483.11917 569.45087 640.90277 | 348.04596 416.20441 514.18146 |
| | | | | | | 649.25629 | 553.28888 651.26447 |
| 602.15452 | 604.1702 | 6.55 | C. elegans | tyglu#6 | C27H30N3O11P | Previously reported (Le et. al. elife | Previously reported (Le et. al. elife |
| | | | C. | | | 2020) | 2020) |
| | | | briggsae | | | | |
| 460.13726 | 462.15291 | 6.34 | C. | tyglu#701 | C19 H28 O10 N P | 53.76678 54.00171 54.50134 54.93059 | 55.05465 69.03373 81.03355 |
| | | | briggsae | | | 78.13238 78.95863 93.67051 93.82211 | 83.04919 101.05945 105.1856 |
| | | | | | | 96.9695 100.04834 142.01453 | 107.07957 118.743 121.06442 |
| | | | | | | 142.87042 149.87952 179.12796 | 127.03864 135.10376 138.09099 |
| | | | | | | 203.14485 255.60117 256.64169 | 220.13248 225.01515 227.09047 |
| | | | | | | 360.08661 378.09726 460.13916 | 228.16006 325.06699 364.17401 |
| | | | | | | | 462.15057 462.23019 |
| 579.17437 | 581.19002 | 8.22 | C. elegans | tyglu#8 | C26 H33 N2 O11 P | 73.35156 78.9585 86.60217 92.05004 | 55.05484 81.03383 83.04948 |
| | | | C. | | | 94.36936 96.96925 99.0448 136.04063 | 92.04974 109.02853 120.04447 |
| | | | briggsae | | | 136.07697 148.96477 197.99699 | 121.06473 122.06006 138.05495 |
| | | | | | | 203.34712 222.3862 222.39954 | 138.09142 202.08633 204.06554 |
| | | | | | | 305.0437 342.07608 360.08615 | 228.06543 257.12805 304.05316 |
| | | | | | | 442.12802 460.13846 579.17603 | 328.11734 346.12814 444.10507 |
| | | | | | | | 483.21225 581.18878 |
| 462.15291 | 464.16856 | 7.56 | C. elegans | tyglu#9 | C19 H30 N O10 P | 51.79475 53.92133 65.89232 75.99543 | 57.07029 69.03375 81.03364 |
| | | | | | | 78.95863 93.68916 93.82409 94.28223 | 85.06483 85.06618 93.77364 |
| | | | | | | 96.96954 105.18489 121.27247 | 97.02823 98.98402 105.17441 |
| | | | | | | 179.86879 179.87704 225.79791 | 118.73328 121.06445 127.03858 |
| | | | | | | 250.76633 257.86258 360.08722 | 138.09096 155.08115 225.01508 |
| | | | | | | 378.09723 388.23373 462.15512 | 229.10617 243.02557 366.18954 |
| | | | | | | | 464.15121 464.16644 |
| 615.13799 | 617.15364 | 11.72 | C. elegans | angl#56 | C28H29N2O12P | 78.95865 86.61275 91.05498 92.05029 | 84.2121 93.74004 107.11538 |
| | | | C. | | | 96.96934 107.17877 123.64606 | 107.11755 120.04405 123.5688 |
| | | | briggsae | | | 135.04578 136.04076 148.96498 | 123.57163 135.11412 155.10469 |
| | | | | | | 162.9808 197.99727 204.99142 | 204.06483 230.4552 232.27657 |
| | | | | | | 223.00186 224.43748 341.04459 | 244.97278 270.96182 295.77274 |
| | | | | | | 360.0506 478.09286 615.14178 | 308.13034 344.3306 375.01569 |
| | | | | | | 640.96216 | 480.1026 551.31396 |
| 547.14816 | 549.16381 | 12.07 | C. elegans | iglu#84 | C25 H29 O10 N2 P | 51.0825 62.63796 63.58872 78.95606 | |
| | | | C. | | | 78.95853 83.49227 94.40952 96.96934 | |
| | | | briggsae | | | 101.06049 105.17789 118.73723 | |
| | | | | | | 124.45099 124.45647 131.06131 | |
| | | | | | | 222.14005 322.04861 424.11761 | |
| | | | | | | 441.14362 459.97211 547.14996 | |

TABLE 4Sb

| | | | | | | Cbr-cest-2 | Cel-cest-1.2 |
|---|---|---|---|---|---|---|---|
| | | | | | | fold over WT | fold over WT |
| | Species the | | | | | (AF16), | (N2), |
| RT | compound was | | Predicted molecular | Putative moieties attached to | Stable isotope | average of 4 | average of 4 |
| (min.) | detected in | SMID | formula | glucose | labeling | repeats | repeats |
| 9.30 | C. elegans C. | angl#10 | C26H26N3O12P | anthranilic acid, nicotinic | | 0.00015689 | 0.00078125 |
| | briggsae | | | acid | | | |
| 7.47 | C. elegans | angl#101 | C19 H21 O11 N2 P | anthranilic acid, nicotinic | | | 0.01274871 |
| | | | | acid | | | |
| 8.02 | C. elegans C. | angl#12 | C16H22NO11P | anthranilic acid, propionic | | 0.001398189 | 0.0042492 |
| | briggsae | | | acid | | | |
| 9.36 | C. elegans C. | angl#161 | C20 H22 O11 N P | anthranilic acid, benzoic acid | | 0.004933872 | 0.00178022 |
| | briggsae | | | | | | |
| 9.64 | C. briggsae | angl#19 | C23 H27 O12 N2 P | anthranilic acid, nicotinic acid, isovaleric acid | | 0.000447547 | |
| 10.38 | C. briggsae | angl#21 | C23 H27 O12 N2 P | anthranilic acid, nicotinic acid, isovaleric acid | | 0.007016209 | |
| 12.17 | C. elegans | angl#22 | C25 H30 O12 N P | anthranilic acid, phenylacetic acid, butyric | | | 0.0046571 |
| | | | | acid | | | |
| 10.24 | C. briggsae | angl#23 | C25 H25 O11 N2 P | antrhanilic acid, nicotinic acid, pyrrolic acid | | 0.001390197 | |
| 12.73 | C. elegans C. | angl#24 | C25 H25 O11 N2 P | antrhanilic acid, nicotinic acid, pyrrolic acid | | 0.00008011 | 0.00003421 |
| | briggsae | | | | | | |
| 11.53 | C. elegans C. | angl#26 | C25 H29 O12 N2 P | anthranilic acid (x2), tiglic | | 1.71E−05 | 0.01182123 |
| | briggsae | | | acid | | | |
| 10.18 | C. elegans C. | angl#27 | C26H25N2O12P | anthranilic acid, benzoic acid, nicotonic acid | | 3.68E−05 | 0.00023137 |
| | briggsae | | | | | | |
| 10.83 | C. elegans C. | angl#28 | C26H25N2O12P | anthranilic acid, benzoic acid, nicotonic acid | | 0.000588909 | 0.00094193 |
| | briggsae | | | | | | |
| 10.60 | C. elegans C. | angl#29 | C26 H25 N2 O12 P | anthranilic acid, nicotinic acid, benzoic acid | | 0.000813258 | 0.00151755 |
| | briggsae | | | | | | |
| 8.80 | C. elegans C. | angl#30 | C25 H24 O12 N3 P | anthranilic acid, nicotinic acid (x2) | | 0.002058623 | 0.00161592 |
| | briaasae | | | | | | |
| 11.80 | C. elegans | angl#32 | C27 H27 O12 N2 P | anthranilic acid (x2), benzoic acid | | | 0.0004297 |
| 7.86 | C. elegans C. | angl#34 | C27 H33 O12 N2 P | anthranilic acid, tiglic acid, phenyalanine | | 0.014949978 | 0.0007289 |
| | briggsae | | | | | | |
| 11.44 | C. elegans C. | angl#36 | C27H28N3O12P | anthranilic acid (x3) | | 1.99E−05 | 0.0003258 |
| | briggsae | | | | | | |
| 8.17 | C. elegans C. | angl#38 | C29 H31 O12 N2 P | anthranilic acid, phenylalanine, benzoic acid | | 0.015032683 | 0.00091898 |
| | briggsae | | | | | | |
| 9.07 | C. elegans C. | angl#40 | C32 H45 O12 N2 P | anthranilic acid (x2), 12:0 | | 0.000713158 | 0.00474321 |
| | briggsae | | | | | | |
| 8.87 | C. elegans C. | angl#401 | C20H23N2O11P | anthranilic acid (x2) | | 0.000526238 | 0.01011689 |
| | briggsae | | | | | | |
| 9.20 | C. elegans C. | angl#41 | C32 H45 O12 N2 P | anthranilic acid (x2), 12:0 | | 0.000409141 | 0.00288662 |
| | briggsae | | | | | | |
| 11.60 | C. elegans | angl#42 | C25 H28 O12 N P | anthranilic acid, tiglic acid, benzoic acid | | | 0.00007037 |
| 9.62 | C. briggsae | angl#44 | C24 H29 O13 N2 P | anthranilic acid (x2), hydroxybutyric acid | | 0.000608287 | |
| 8.64 | C. elegans C. | angl#46 | C17 H24 O11 N P | anthranilic acid, isovaleric | | 0.00024863 | 0.01675484 |
| | briggsae | | | acid | | | |
| 10.37 | C. elegans C. | angl#47 | C27 H27 O13 N2 P | anthranilic acid (x2), hydroxybenzoic acid | | 0.000548872 | 0.00395672 |
| | briggsae | | | | | | |
| 9.60 | C. elegans C. | angl#48 | C27 H27 O13 N2 P | anthranilic acid (x2), hydroxybenozic acid | | 0.000470152 | 0.01184122 |
| | briggsae | | | | | | |
| 10.44 | C. elegans C. | angl#50 | C28 H29 O13 N2 P | anthranilic acid (x2), hydroxy phenylacetic | | 0.000189734 | 0.00747852 |
| | briggsae | | | acid | | | |
| 11.11 | C. elegans C. | angl#51 | C25 H26 O12 N3 P | anthranilic acid (x2), pyrrolic acid | | 0.0039433 | 0.00581656 |
| | briggsae | | | | | | |
| 10.84 | C. elegans C. | angl#52 | C25 H26 O12 N3 P | anthranilic acid (x2), pyrrolic acid | | 0.00037602 | 0.00617927 |
| | briggsae | | | | | | |
| 11.59 | C. briggsae | angl#54 | C23 H32 O12 N P | anthranilic acid, tiglic acid, isovaleric acid | | 0.000674363 | |
| 11.72 | C. briggsae | angl#56 | C28H29N2O12P | anthranilic acid (x2), phenylacetic acid | | 0.000458711 | |
| 9.16 | C. elegans C. | angl#601 | C18 H24 O11 N P | anthranilic acid, tiglic acid | | 0.005194057 | 0.00985428 |
| | briggsae | | | | | | |
| 11.87 | C. elegans C. | angl#70 | C27 H26 N O12 P | anthranilic acid, benzoic acid (x2) | | 8.68E−05 | 0.00055226 |
| | briggsae | | | | | | |
| 11.53 | C. elegans | bzglu#10 | C25 H24 O12 N P | benzoic acid (x2), pyrrolic | | | 0.00047799 |
| | | | | acid | | | |
| 9.58 | C. elegans C. | bzglu#12 | C24 H23 O12 N2 P | benzoic acid, nicotinic acid, pyrrolic acid | | 0.003120369 | 0.00701645 |
| | briggsae | | | | | | |
| 10.23 | C. elegans | bzglu#13 | C24 H23 O12 N2 P | benzoic acid, nicotinic acid, pyrrolic acid | | | 0.00374687 |
| 9.62 | C. elegans C. | bzglu#201 | C18 H23 O11 P | benozic acid, tiglic acid | | 0.002390755 | 0.00180659 |
| | briggsae | | | | | | |
| 8.98 | C. elegans | bzglu#4 | C24 H28 O12 N P | benzoic acid, nicotinic acid, isovaleric acid | | | 0.00338709 |
| 10.87 | C. elegans C. | bzglu#6 | C25H23N2O12P | benzoic acid (x2), nicotinic | | 0.00313275 | 0.01304709 |
| | briggsae | | | acid | | | |
| 10.15 | C. elegans | bzglu#8 | C25 H29 O12 P | benzoic acid (x2), isovaleric | | | 0.00062483 |
| | | | | acid | | | |
| 10.63 | C. elegans C. | bzglu#9 | C23 H23 O12 N2 P | benzoic acid, pyrrolic acid | | 0.0036796 | 0.00062982 |
| | briggsae | | | (x2) | | | |
| 10.88 | C. elegans | higlas#7 | C34 H42 O15 N P | hydroxyindole, ascr#7, benzoic acid | | | 0.00029757 |
| 10.28 | C. elegans C. | higlu#3 | C27H25N2O11P | hydoxyindole, nicotinc acid, benzoic acid | | 0.000477308 | 0.00118222 |
| | briggsae | | | | | | |
| 10.85 | C. elegans | higlu#4 | C27H25N2O11P | hydoxyindole, nicotinc acid, benzoic acid | | | 0.00400449 |
| 11.80 | C. elegans | iglas#101 | C34 H44 O14 N P | indole, ascr#1, benzoic acid | | | 0.00078811 |
| 12.48 | C. elegans | iglas#301 | C36 H46 O14 N P | indole, ascr#3, benzoic acid | | | 0.00055057 |
| 11.36 | C. elegans | iglas#701 | C34 H43 O14 N2 P | indole, ascr#7, anthranilic | | | 0.00034775 |
| | | | | acid | | | |
| 11.63 | C. elegans | iglas#702 | C34 H42 O14 N P | indole, ascr#7, benzoic acid | | | 0.00036956 |
| 9.48 | C. elegans C. | iglu#101 | C19H21N2O9P | indole, pyrrolic acid | | 0.01530168 | 0.00027683 |
| | briggsae | | | | | | |
| 10.28 | C. elegans C. | iglu#121 | C21H22NO9P | indole, benzoic acid | | 5.40E−05 | 0.00062771 |
| | briggsae | | | | | | |
| 10.54 | C. elegans C. | iglu#141 | C19 H26 O9 N P | indole, isovaleric acid | 13C5 from | 0.000179285 | 0.0009611 |
| | briggsae | | | | 13C6-L- | | |
| | | | | | leucine | | |
| 10.78 | C. elegans C. | iglu#16 | C22 H24 O9 N P | indole, phenyacetic acid | | 0.001912174 | 0.00053305 |
| | briggsae | | | | | | |
| 9.31 | C. elegans | iglu#181 | C21 H22 O10 N P | indole, hydroxybenzoic acid | | | 0.00491616 |
| 11.18 | C. elegans | iglu#19 | C23 H25 O10 N2 P | indole, propionic acid, nicotinic acid | | | 0.00261954 |
| 10.31 | C. elegans C. | iglu#21 | C23 H25 O10 N2 P | indole, propionic acid, nicotinic acid | | 0.00222046 | 0.01359998 |
| | briggsae | | | | | | |
| 12.29 | C. elegans | iglu#22 | C24 H27 O10 N2 P | indole, pyrrolic acid, tiglic | | | 0.00016186 |
| | | | | acid | | | |
| 10.52 | C. elegans | iglu#23 | C23 H25 O10 N2 P | indole, propionic acid, nicotinic acid | | | 0.00182517 |
| 11.85 | C. elegans C. | iglu#24 | C24 H27 O10 N2 P | indole, anthranilic acid, propionic acid | | 0.008042815 | 0.0007803 |
| | briggsae | | | | | | |
| 10.89 | C. elegans | iglu#25 | C23 H25 O10 N2 P | indole, propionic acid, nicotinic acid | | | 0.00446209 |
| 11.53 | C. elegans | iglu#27 | C24 H24 O10 N3 P | indole, pyrrolic acid (x2) | | | 0.00014603 |
| 13.04 | C. elegans C. | iglu#28 | C26 H28 O10 N P | indole, benzoic acid, tiglic | | 0.019574324 | 0.00009358 |
| | briggsae | | | acid | | | |
| 11.02 | C. elegans | iglu#30 | C25 H27 O10 N2 P | indole, nicotinic acid, tiglic | | | 0.00028374 |
| | | | | acid | | | |
| 11.84 | C. elegans C. | iglu#31 | C25 H27 O10 N2 P | indole, nicotinic acid, tiglic | | 0.00010709 | 0.00140268 |
| | briggsae | | | acid | | | |
| 13.40 | C. elegans | iglu#32 | C26 H30 O10 N P | indole, benzoic acid, isovaleric acid | | | 0.00143677 |
| 13.58 | C. elegans | iglu#34 | C26 H30 O10 N P | indole, benzoic acid, isovaleric acid | | | 0.00386348 |
| 11.81 | C. elegans | iglu#36 | C25H29N2O10P | indole, nicotinic acid, isovaleric acid | | | 0.00336836 |
| 12.24 | C. elegans | iglu#37 | C26 H25 O10 N2 P | indole, pyrrolic acid, benzoic acid | | | 0.00011688 |
| 12.53 | C. elegans | iglu#38 | C26 H25 O10 N2 P | indole, pyrrolic acid, benzoic acid | | | 0.0000986 |
| 10.78 | C. elegans | iglu#39 | C25 H24 O10 N3 P | indole, nicotinic acid, pyrrolic acid | | | 0.00031443 |
| 10.50 | C. elegans C. | iglu#40 | C25 H24 O10 N3 P | indole, nicotinic acid, pyrrolic acid | | 0.00219105 | 0.00016899 |
| | briggsae | | | | | | |
| 9.94 | C. elegans C. | iglu#401 | C21H23N2O9P | indole, anthranilic acid | | 3.71E−05 | 0.0009932 |
| | briggsae | | | | | | |
| 10.95 | C. elegans C. | iglu#41 | C27 H26 O10 N3 P | indole, nicotinic acid, anthranilic acid | | 0.000369129 | 0.00006705 |
| | briggsae | | | | | | |
| 10.49 | C. elegans C. | iglu#41 | C27H26N3O10P | indole, anthranilic acid, nicotinic acid | | 7.32876E−05 | 0.00071115 |
| | briggsae | | | | | | |
| 10.48 | C. elegans C. | iglu#42 | C26H29N2O10P | indole, antranilic acid, tiglic | | 0.008524144 | 0.00162427 |
| | briggsae | | | acid | | | |
| 13.07 | C. elegans | iglu#44 | C26 H31 O10 N2 P | indole, anthranilic acid, isovaleric acid | | | 0.00065121 |
| 9.79 | C. elegans | iglu#45 | C25H30N3O10P | indole, isovaleric acid, uraconic acid | | | 0.00108903 |
| 9.60 | C. elegans | iglu#46 | C25H30N3O10P | indole, isovaleric acid, uraconic acid | | | 0.00088881 |
| 10.89 | C. elegans | iglu#47 | C25 H29 O11 N2 P | indole, anthranilic acid, hydroxybutirc acid | | | 0.00074685 |
| 10.50 | C. briggsae | iglu#48 | C25 H29 O11 N2 P | indole, anthranilic acid, hydroxybutirc acid | | 0.002594803 | |
| 11.42 | C. briggsae | iglu#49 | C25 H29 O11 N2 P | indole, anthranilic acid, hydroxybutirc acid | | 0.006782981 | |
| 13.26 | C. elegans C. | iglu#50 | C28 H26 O10 N P | indole, benzoic acid (x2) | | 9.54E−05 | 0.00005754 |
| | briggsae | | | | | | |
| 11.37 | C. elegans C. | iglu#52 | C27 H25 O10 N2 P | indole, benzoic acid, nicotinic acid | | 0.00003754 | 0.00007449 |
| | briggsae | | | | | | |
| 12.10 | C. elegans | iglu#53 | C27 H25 O10 N2 P | indole, benzoic acid, nicotinic acid | | | 0.00259227 |
| 11.73 | C. elegans | iglu#54 | C27 H25 O10 N2 P | indole, benzoic acid, nicotinic acid | | | 0.00032058 |
| 9.93 | C. elegans C. | iglu#56 | C26H24N3O10P | indole, nicotinic acid (x2) | | 0.00167939 | 0.00912717 |
| | briggsae | | | | | | |
| 12.13 | C. elegans C. | iglu#57 | C26 H26 O10 N3 P | indole, anthranilic acid, pyrrolic acid | | 0.006392733 | 0.00024194 |
| | briggsae | | | | | | |
| 11.94 | C. elegans | iglu#58 | C26 H26 O10 N3 P | indole, anthranilic acid, pyrrolic acid | | | 0.00018016 |
| 11.10 | C. elegans | iglu#60 | C26 H25 O11 N2 P | indole, hydroxybenzoic acid, pyrrolic acid | | | 0.00039722 |
| 8.30 | C. elegans C. | iglu#601 | C20H21N2O9P | indole, nicotinic acid | | 0.009609961 | 0.00184486 |
| | briggsae | | | | | | |
| 9.30 | C. elegans | iglu#62 | C25 H25 O10 N4 P | indole, uraconic acid, pyrrolic acid | | | 0.00181808 |
| 13.45 | C. elegans | iglu#64 | C29 H28 O10 NP | indole, benzoic acid, phenylacetic acid | | | 0.00042477 |
| 12.62 | C. elegans C. | iglu#65 | C28 H27 O10 N2 P | indole, benzoic acid, anthranilic acid | | 5.36E−05 | 0.00834225 |
| | briggsae | | | | | | |
| 12.95 | C. elegans C. | iglu#66 | C28 H27 O10 N2 P | indole, benzoic acid, anthranilic acid | | 0.012388214 | 0.00012061 |
| | briggsae | | | | | | |
| 11.89 | C. elegans | iglu#68 | C28 H27 O10 N2 P | indole, nictonic acid, phenylacetic acid | | | 0.00109184 |
| 9.81 | C. elegans C. | iglu#69 | C27 H26 O10 N3 P | indole, benzoic acid, uraconic acid | | 0.000832077 | 0.01463814 |
| | briggsae | | | | | | |
| 9.93 | C. elegans C. | iglu#70 | C27H26N3O10P | indole, benzoic acid, uraconic acid | | 0.01157415 | 0.00390362 |
| | briggsae | | | | | | |
| 13.16 | C. elegans C. | iglu#72 | C29 H29 O10 N2 P | indole, anthranilic acid, phenylacetic acid | | 0.00181527 | 0.00094034 |
| | briggsae | | | | | | |
| 12.54 | C. elegans C. | iglu#74 | C28H28N3O10P | indole, anthranilic acid (x2) | | 4.29E−05 | 0.00003983 |
| | briggsae | | | | | | |
| 11.23 | C. elegans C. | iglu#75 | C28 H27 O11 N2 P | indole, anthranilic acid, hydroxybenozic acid | | 0.00199282 | 0.01522004 |
| | briggsae | | | | | | |
| 11.48 | C. elegans C. | iglu#76 | C28 H27 O11 N2 P | indole, anthranilic acid, hydroxybenozic acid | | 0.000713997 | 0.00023001 |
| | briggsae | | | | | | |
| 10.04 | C. elegans C. | iglu#801 | C19H24NO9P | indole, tiglic acid | | 8.69E−05 | 0.00071761 |
| | briggsae | | | | | | |
| 9.78 | C. elegans C. | iglu#82 | C27 H27 O10 N4 P | indole, anthranilic acid, uraconic acid | | 0.001492565 | 0.00110764 |
| | briggsae | | | | | | |
| 11.33 | C. elegans C. | iglu#86 | C29 H29 O11 N2 P | indole, anthranilic acid, hydroxylphenylacetic | | 0.00034561 | 0.01799293 |
| | briggsae | | | acid | | | |
| 12.37 | C. elegans C. | iglu#88 | C26 H29 O10 N2 P | indole, anthranilic acid, tiglic acid | | 8.01E−05 | 0.00003421 |
| | briggsae | | | | | | |
| 11.70 | C. elegans C. | iglu#90 | C28 H27 O10 N2 P | indole, anthranilic acid, benzoic acid | | 0.002597042 | 0.00193921 |
| | briggsae | | | | | | |
| 10.50 | C. elegans C. | iglu#92 | C24 H27 O10 N2 P | indole, nicotinic acid, butyric acid | | 0.001118316 | 0.00800221 |
| | briggsae | | | | | | |
| 5.35 | C. elegans | mgglu#201 | C19 H22 O10 N5 P | methyl guanine, benzoic acid | | | 0.00382139 |
| 7.63 | C. elegans | mgglu#4 | C26 H26 O10 N5 P | methyl adenine, benzoic acid (x2) | | | 0.00443418 |
| 8.12 | C. elegans C. | mgglu#6 | C26H28N5O11P | methyl guanine, benzoic acid (x2) | | 0.0002185 | 0.00039704 |
| | briggsae | | | | | | |
| 9.85 | C. elegans C. | nglu#10 | C25 H30 O12 N P | nicotinic acid, isovaleric acid, phenylacetic | | 0.005457544 | 0.00208349 |
| | briggsae | | | acid | | | |
| 10.37 | C. elegans C. | nglu#3 | C24H26NO12P | nicotinic acid, benzoic acid, tiglic acid | | 0.000553682 | 0.0038265 |
| | briggsae | | | | | | |
| 10.60 | C. elegans C. | nglu#4 | C24H26NO12P | nicotinic acid, benzoic acid, tiglic acid | | 0.00724701 | 0.00161073 |
| | briggsae | | | | | | |
| 9.39 | C. elegans C. | nglu#5 | C23 H25 O12 N2 P | nicotinc acid (x2), tiglic acid | | 0.00454593 | 0.00398701 |
| | briggsae | | | | | | |
| 9.09 | C. elegans C. | nglu#6 | C23 H25 O12 N2 P | nicotinc acid (x2), tiglic acid | | 0.01335601 | 0.01379546 |
| | briggsae | | | | | | |
| 9.80 | C. elegans C. | nglu#7 | C24H27N2O12P | nicotinic acid (x2), 6:1 | | 0.000219567 | 0.00896224 |
| | briggsae | | | | | | |
| 9.89 | C. elegans | nglu#8 | C24H27N2O12P | nicotinic acid (x2), 6:1 | | | 0.0042435 |
| 10.24 | C. elegans C. | nglu#9 | C24H27N2O12P | nicotinic acid (x2), 6:1 | | 0.001271617 | 0.00145121 |
| | briggsae | | | | | | |
| 7.48 | C. elegans C. | oglu#10 | C26 H33 O12 N2 P | octopamine, anthranilic acid, | d1 from d2-L- | 0.004160164 | 0.00254476 |
| | briggsae | | | tiglic acid | tyrosine | | |
| 6.89 | C. elegans C. | oglu#1401 | C26 H30 O12 N3 P | octopamine, anthranilic acid, pyrrolic acid | | 0.01397622 | 0.01958842 |
| | briggsae | | | | | | |
| 7.76 | C. elegans C. | oglu#15 | C27 H30 O12 N3 P | octopamine, anthranilic acid, nicotinic acid | | 0.003824015 | 0.00072639 |
| | briggsae | | | | | | |
| 6.48 | C. elegans | oglu#16 | C27 H30 O12 N3 P | octopamine, anthranilic acid nicotinic acid | | | 0.00074573 |
| 7.00 | C. elegans | oglu#17 | C28 H31 O13 N2 P | octopamine, anthranilic acid, hydroxybenzoic | | | 0.00150513 |
| | | | | acid | | | |
| 8.12 | C. elegans C. | oglu#22 | C29 H39 O13 N2 P | octopamine, anthranilic acid, m6:1 | | 0.000583041 | 0.00063116 |
| | briggsae | | | | | | |
| 6.86 | C. briggsae | oglu#24 | C27 H29 O12 N2 P | octopamine, nicotinic acid, benzoic acid | | 0.00336156 | |
| 6.68 | C. elegans | oglu#26 | C24 H28 O12 N3 P | octopamine, pyrrolic acid | | | 0.01263528 |
| | | | | (x2) | | | |
| 5.88 | C. briggsae | oglu#4 | C21 H26 N2 O11 P | octopamine, anthranilic acid | | 0.00769982 | |
| 6.11 | C. briggsae | oglu#401 | C21 H26 N2 O11 P | octopamine, anthranilic acid | | 0.00769982 | |
| 7.21 | C. elegans | oglu#601 | C23 H27 O13 N2 P | octopamine, tiglic acid, pyrrolic acid | | | 0.00227689 |
| 7.92 | C. elegans | oglu#7 | C26 H29 O12 N2 P | octopamine, benzoic acid, pyrrolic acid | | | 0.00147204 |
| 7.51 | C. elegans | oglu#8 | C26 H29 O12 N2 P | octopamine, benzoic acid, pyrrolic acid | | | 0.0174488 |
| 7.80 | C. elegans | oglu#9 | C26 H33 O12 N2 P | octopamine, anthranilic acid, | d1 from d2-L- | | 0.00029126 |
| | | | | tiglic acid | tyrosine | | |
| 8.77 | C. elegans C. | pyglu#201 | C18 H20 O11 N P | pyrrolic acid benzoic acid | | 0.004461916 | 0.00184397 |
| | briggsae | | | | | | |
| 11.22 | C. elegans C. | pyglu#4 | C23 H26 O12 N P | pyrrolic acid benzoic acid, tiglic acid | | 0.003756665 | 0.0071366 |
| | briggsae | | | | | | |
| 7.95 | C. elegans | tyglas#1 | C34 H49 O15 N2 P | tyramine, ascr#1, anthranilic acid | | | 0.00084111 |
| 7.23 | C. elegans C. | tyglas#11 | C31 H43 O15 N2 P | tyramine, ascr#11, anthranilic acid | | 0.001146292 | 0.00578661 |
| | briggsae | | | | | | |
| 7.03 | C. briggsae | tyglas#5 | C30 H41 O15 N2 P | tyramine, ascr#5, anthranilic acid | | 0.001127165 | |
| 7.89 | C. elegans | tyglas#7 | C34 H47 O15 N2 P | tyramine, ascr#7, anthranilic acid | | | 0.00070889 |
| 5.95 | C. briggsae | tyglas#9 | C25 H40 O14 N P | tyramine, ascr#9 | | 0.019071257 | |
| 8.63 | C. elegans C. | tyglu#12 | C29 H33 O11 N2 P | tyramine, anthranilic acid, | d2 from d2-L- | 0.000236654 | 0.00881819 |
| | briggsae | | | phenylacetic acid | tyrosine | | |
| 6.58 | C. briggsae | tyglu#131 | C19 H30 NO 10 P | tyramine, isovaleric acid | 13C5 from | 0.001613548 | |
| | | | | | 13C6-L- | | |
| | | | | | leucine | | |
| 8.37 | C. elegans C. | tyglu#14 | C26 H35 O11 N2 P | tyramine, isovaleric acid, | 13C5 from | 0.005026996 | 0.0004346 |
| | briggsae | | | anthranilic acid | 13C6-L- | | |
| | | | | | leucine | | |
| 7.97 | C. elegans C. | tyglu#16 | C28H31N2O11P | tyramine, anthranilic acid, | d2 from d2-L- | 0.00004669 | 4.85085E−05 |
| | briggsae | | | brenzoic acid | Tyrosine | | |
| 5.92 | C. elegans C. | tyglu#181 | C19 H25 O10 N2 P | tyramine, pyrrolic acid | d2 from d2-L- | 0.00488738 | 0.00175459 |
| | briggsae | | | | tyrosine | | |
| 7.98 | C. elegans | tyglu#19 | C24 H31 O11 N2 P | tyramine, pyrrolic acid, tiglic | d2 from d2-L- | | 0.00035181 |
| | | | | acid | tyrosine | | |
| 6.27 | C. elegans | tyglu#2 | C21H27N2O10P | tyramine, anthranilic acid | d2 from d2-L- | | 0.00035508 |
| | | | | | tyrosine | | |
| 7.46 | C. elegans C. | tyglu#20 | C24 H31 O11 N2 P | tyramine, pyrrolic acid, tiglic | d2 from d2-L- | 0.000670797 | 0.00035181 |
| | briggsae | | | acid | tyrosine | | |
| 8.13 | C. elegans C. | tyglu#22 | C24 H33 O11 N2 P | tyramine, pyrrolic acid, | d2 from d2-L- | 0.00658276 | 0.00321667 |
| | briggsae | | | isovaleric acid | tyrosine | | |
| 7.42 | C. elegans | tyglu#24 | C24 H28 O11 N3 P | tyramine, pyrrolic acid (x2) | d2 from d2-L- | | 0.01672344 |
| | | | | | tyrosine | | |
| 8.38 | C. briggsae | tyglu#26 | C26 H32 O11 N P | tyramine, benzoic acid, tiglic acid | | 0.015208658 | |
| 8.17 | C. elegans C. | tyglu#28 | C26 H29 O11 N2 P | tyramine, pyrrolic acid, benzoic acid | | 0.009782743 | 0.00020549 |
| | briggsae | | | | | | |
| 6.88 | C. elegans C. | tyglu#30 | C25 H28 O11 N3 P | tyramine, pyrrolic acid, nicotinc acid | | 0.00298485 | 0.0031946 |
| | briggsae | | | | | | |
| 8.61 | C. elegans C. | tyglu#32 | C28 H30 O11 N P | tyramine, benzoic acid (x2) | | 0.001228069 | 0.00754644 |
| | briggsae | | | | | | |
| 7.70 | C. elegans C. | tyglu#34 | C26 H30 O11 N3 P | tyramine, anthranilic acid, | d2 from d2-L- | 0.005293702 | 0.00032706 |
| | briggsae | | | pyrrolic acid | tyrosine | | |
| 8.17 | C. elegans C. | tyglu#35 | C28 H31 O12 N2 P | tyramine, anthranilic acid, hydroxybenzoic | | 0.002264335 | 0.00174368 |
| | briggsae | | | acid | | | |
| 7.64 | C. elegans | tyglu#36 | C28 H31 O12 N2 P | tyramine, anthranilic acid, | d2 from d2-L- | | 0.00165659 |
| | | | | hydroxybenzoic acid | tyrosine | | |
| 7.81 | C. elegans C. | tyglu#37 | C29 H33 O12 N2 P | tyramine, anthranilic acid, | d2 from d2-L- | 0.000234764 | 0.01231304 |
| | briggsae | | | hydroxy-phenylacetic acid | tyrosine | | |
| 6.28 | C. elegans C. | tyglu#38 | C27 H31 O11 N4 P | tyramine, uraconic acid, anthranilic acid | | 0.001217353 | 0.00195793 |
| | briggsae | | | | | | |
| 7.65 | C. elegans C. | tyglu#4 | C28H32N3O11P | tyramine, anthranilic acid | | 0.00001992 | 0.00040621 |
| | briggsae | | | (x2) | | | |
| 7.00 | C. elegans C. | tyglu#42 | C30 H36 O11 N3 P | tyramine, anthranilic acid, phenylalanine | | 0.000188334 | 0.00434918 |
| | briggsae | | | | | | |
| 8.20 | C. elegans C. | tyglu#45 | C29 H39 O13 N2 P | tyramine, anthranilic acid, | | 0.000681255 | 0.01913772 |
| | briggsae | | | d8:2 | | | |
| 10.32 | C. elegans C. | tyglu#50 | C31 H39 O11 N2 P | tyramine, anthranilic acid, | | 0.00293829 | 0.00889652 |
| | briggsae | | | 10:3 | | | |
| 5.38 | C. briggsae | tyglu#501 | C20 H25 O10 N2 P | tyramine, nicotinic acid | d2 from d2-L- | 0.001880109 | |
| | | | | | tyrosine | | |
| 7.90 | C. elegans C. | tyglu#52 | C25 H33 O11 N2 P | tyramine, anthranilic acid | 13C5 from | 0.000158388 | 0.00950817 |
| | briggsae | | | isovaleric acid | 13C6-L- | | |
| | | | | | leucine | | |
| 7.75 | C. briggsae | tyglu#53 | C25 H40 O11 N P | tyramine, m11:1 | | 0.002603976 | |
| 11.07 | C. briggsae | tyglu#54 | C25H40NO10P | tyramine, 11:1 | | 0.00211232 | |
| 10.26 | C. briggsae | tyglu#56 | C31 H43 O11 N2 P | tyramine, nicotinic acid, 11:1 | | 0.000526302 | |
| 6.55 | C. elegans C. | tyglu#6 | C27H30N3O11P | tyramine, anthranilic acid, | d2 from d2-L- | 0.00005252 | 0.00020897 |
| | briggsae | | | nicotinic acid | Tyrosine | | |
| 6.34 | C. briggsae | tyglu#701 | C19 H28 O10 N P | tyramine, tiglic acid | d2 from d2-L- | 0.001210488 | |
| | | | | | tyrosine | | |
| 8.22 | C. elegans C. | tyglu#8 | C26 H33 N2 O11 P | tyramine, anthranilic acid, | d2 from d2-L- | 0.000184056 | 0.00011681 |
| | briggsae | | | tiglic acid | tyrosine | | |
| 7.56 | C. elegans | tyglu#9 | C19 H30 N O10 P | tyramine, isovaleric acid | | | 0.0030417 |
| 11.72 | C. elegans C. | angl#56 | C28H29N2O12P | antranilic acid (x2), phenylacetic acid | | 0.000458711 | 0.010779114 |
| | briggsae | | | | | | |
| 12.07 | C. elegans C. | iglu#84 | C25 H29 O10 N2 P | indole, anthranilic acid, butyric acid | | 0.000177329 | 0.001499089 |
| | briggsae | | | | | | |

TABLE S5
For compounds bearing a pyranosidyl core with undefined stereochemistry, it will be appreciated that the present disclosure encompasses all
stereoisomers including that having the stereochemical assignment of glucose.

| Structure | SMID |
|---|---|
| 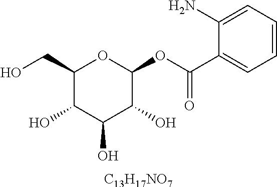 [SMILES: Nc1ccccc1C(=O)O[C@@H]1O[C@H](CO)[C@@H](O)[C@H](O)[C@H]1O ; "C13H17NO7"] | angl#1 |
| 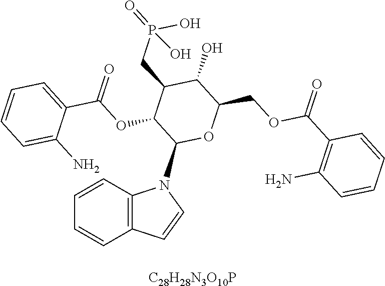 | iglu#74 |
| 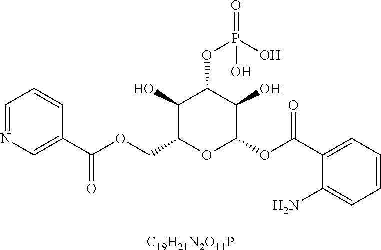 [SMILES: Nc1ccccc1C(=O)O[C@@H]1O[C@H](COC(=O)c2cccnc2)[C@@H](O)[C@H](OP(=O)(O)O)[C@H]1O ; "C19H21N2O11P"] | angl#10 |
| 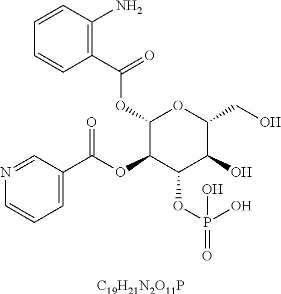 [SMILES: Nc1ccccc1C(=O)O[C@@H]1O[C@H](CO)[C@@H](O)[C@H](OP(=O)(O)O)[C@H]1OC(=O)c1cccnc1 ; "C19H21N2O11P"] | angl#101 |
| 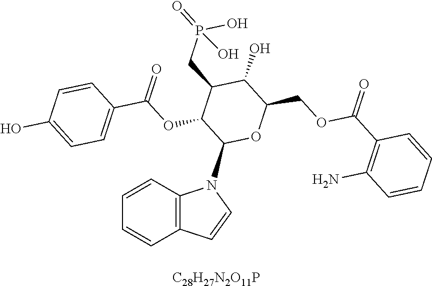 [SMILES: Nc1ccccc1C(=O)OC[C@H]1O[C@@H](n2ccc3ccccc32)[C@H](OC(=O)c2ccc(O)cc2)[C@@H](CP(=O)(O)O)[C@@H]1O ; "C28H27N2O11P"] | iglu#75 |
| 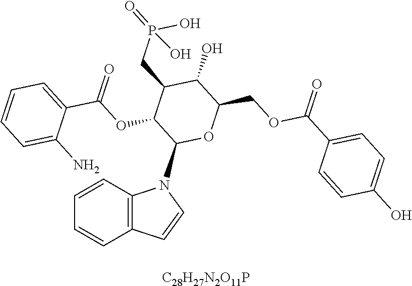 [SMILES: Nc1ccccc1C(=O)O[C@@H]1[C@@H](CP(=O)(O)O)[C@H](O)[C@@H](COC(=O)c2ccc(O)cc2)O[C@H]1n1ccc2ccccc21 ; "C28H27N2O11P"] | iglu#76 |
| 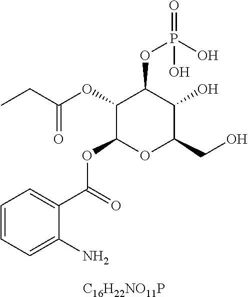 | angl#12 |
| 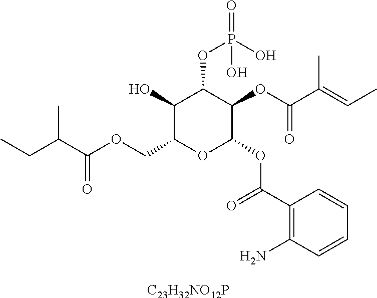 [SMILES: C/C=C(\C)C(=O)O[C@H]1[C@H](OC(=O)c2ccccc2N)O[C@H](COC(=O)C(C)CC)[C@@H](O)[C@@H]1OP(=O)(O)O ; "C23H32NO12P"] | angl#18 |
| 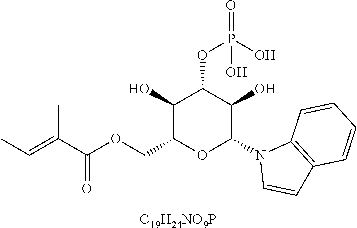 [SMILES: C/C=C(\C)C(=O)OC[C@H]1O[C@@H](n2ccc3ccccc32)[C@H](O)[C@@H](OP(=O)(O)O)[C@@H]1O ; "C19H24NO9P"] | iglu#8 |
| 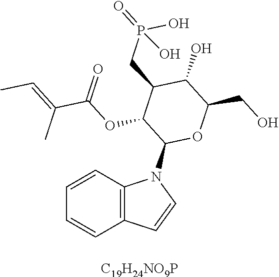 | iglu#801 |
| 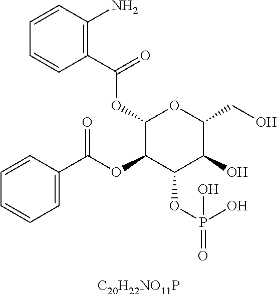 [SMILES: Nc1ccccc1C(=O)O[C@@H]1O[C@H](CO)[C@@H](O)[C@H](OP(=O)(O)O)[C@H]1OC(=O)c1ccccc1 ; "C20H22NO11P"] | angl#161 |
| 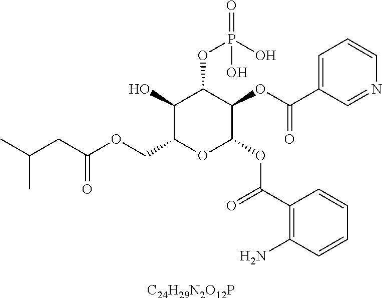 [SMILES: CC(C)CC(=O)OC[C@H]1O[C@@H](OC(=O)c2ccccc2N)[C@H](OC(=O)c2cccnc2)[C@@H](OP(=O)(O)O)[C@@H]1O ; "C24H29N2O12P"] | angl#19 |
| 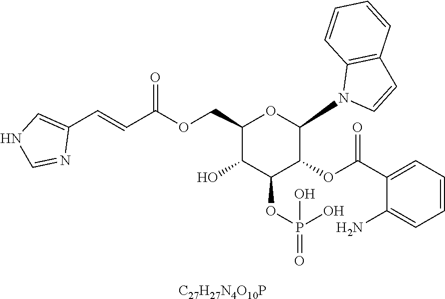 [SMILES: Nc1ccccc1C(=O)O[C@@H]1[C@@H](OP(=O)(O)O)[C@H](O)[C@@H](COC(=O)/C=C/c2c[nH]cn2)O[C@H]1n1ccc2ccccc21 ; "C27H27N4O10P"] | iglu#82 |
| 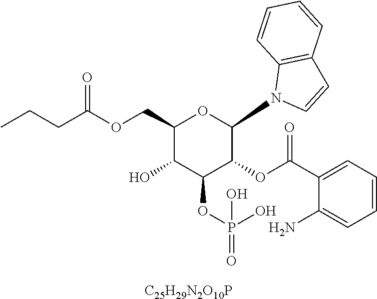 [SMILES: CCCC(=O)OC[C@H]1O[C@@H](n2ccc3ccccc32)[C@H](OC(=O)c2ccccc2N)[C@@H](OP(=O)(O)O)[C@@H]1O ; "C25H29N2O10P"] | iglu#84 |
| 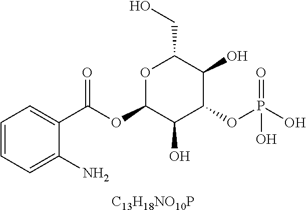 | angl#2 |
| 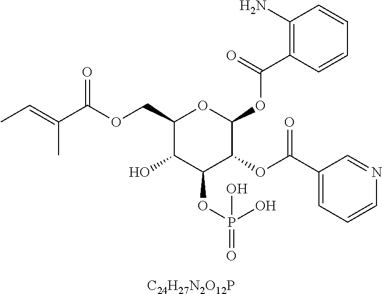 [SMILES: C/C=C(\C)C(=O)OC[C@H]1O[C@@H](OC(=O)c2ccccc2N)[C@H](OC(=O)c2cccnc2)[C@@H](OP(=O)(O)O)[C@@H]1O ; "C24H27N2O12P"] | angl#20 |
| 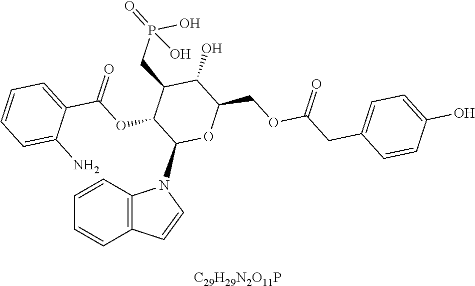 [SMILES: Nc1ccccc1C(=O)O[C@@H]1[C@@H](CP(=O)(O)O)[C@H](O)[C@@H](COC(=O)Cc2ccc(O)cc2)O[C@H]1n1ccc2ccccc21 ; "C29H29N2O11P"] | iglu#86 |
| 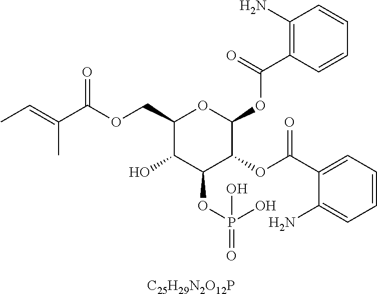 [SMILES: C/C=C(\C)C(=O)OC[C@H]1O[C@@H](OC(=O)c2ccccc2N)[C@H](OC(=O)c2ccccc2N)[C@@H](OP(=O)(O)O)[C@@H]1O ; "C25H29N2O12P"] | iglu#88 |
| 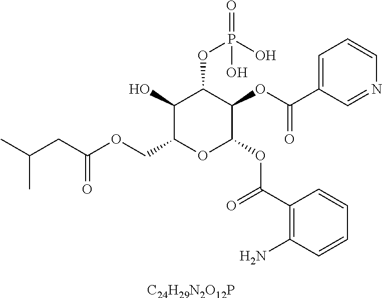 [SMILES: CC(C)CC(=O)OC[C@H]1O[C@@H](OC(=O)c2ccccc2N)[C@H](OC(=O)c2cccnc2)[C@@H](OP(=O)(O)O)[C@@H]1O ; "C24H29N2O12P"] | angl#21 |
| 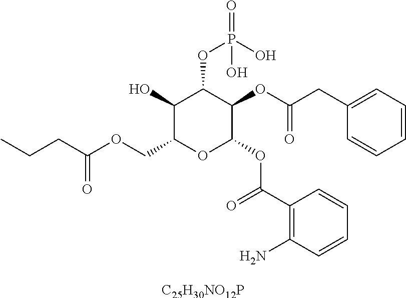 [SMILES: CCCC(=O)OC[C@H]1O[C@@H](OC(=O)c2ccccc2N)[C@H](OC(=O)Cc2ccccc2)[C@@H](OP(=O)(O)O)[C@@H]1O ; "C25H30NO12P"] | angl#22 |
| 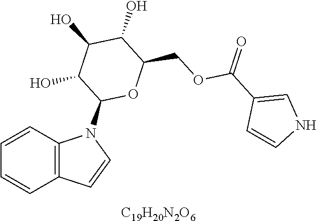 | iglu#9 |
| 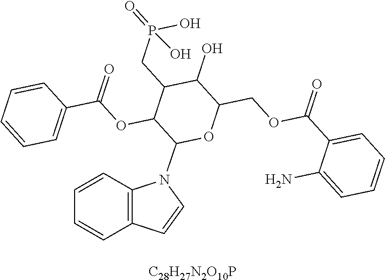 [SMILES: Nc1ccccc1C(=O)OCC1OC(n2ccc3ccccc32)C(OC(=O)c2ccccc2)C(CP(=O)(O)O)C1O ; "C28H27N2O10P"] | iglu#90 |
| 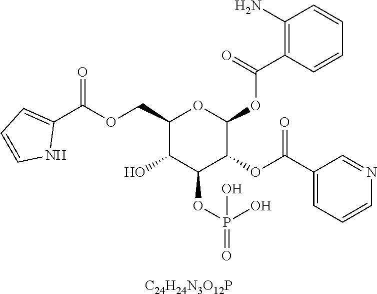 [SMILES: Nc1ccccc1C(=O)O[C@@H]1O[C@H](COC(=O)c2ccc[nH]2)[C@@H](O)[C@H](OP(=O)(O)O)[C@H]1OC(=O)c1cccnc1 ; "C24H24N3O12P"] | angl#23 |
| 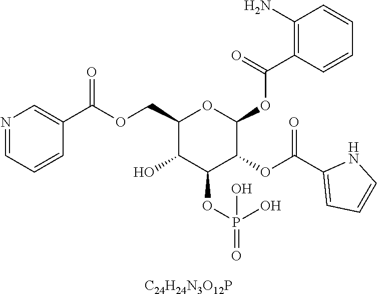 [SMILES: Nc1ccccc1C(=O)O[C@@H]1O[C@H](COC(=O)c2cccnc2)[C@@H](O)[C@H](OP(=O)(O)O)[C@H]1OC(=O)c1ccc[nH]1 ; "C24H24N3O12P"] | angl#24 |
| 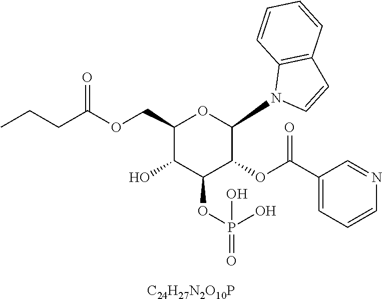 [SMILES: CCCC(=O)OC[C@H]1O[C@@H](n2ccc3ccccc32)[C@H](OC(=O)c2cccnc2)[C@@H](OP(=O)(O)O)[C@@H]1O ; "C24H27N2O10P"] | iglu#92 |
| 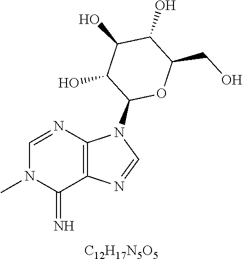 [SMILES: Cn1cnc2c(ncn2[C@@H]2O[C@H](CO)[C@@H](O)[C@H](O)[C@H]2O)c1=N ; "C12H17N5O5"] | maglu#1 |
| 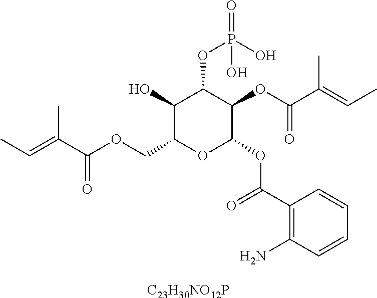 [SMILES: C/C=C(\C)C(=O)OC[C@H]1O[C@@H](OC(=O)c2ccccc2N)[C@H](OC(=O)/C(C)=C/C)[C@@H](OP(=O)(O)O)[C@@H]1O ; "C23H30NO12P"] | angl#26 |
| 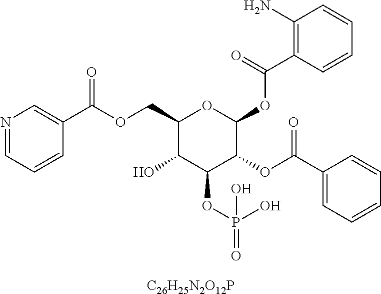 [SMILES: Nc1ccccc1C(=O)O[C@@H]1O[C@H](COC(=O)c2cccnc2)[C@@H](O)[C@H](OP(=O)(O)O)[C@H]1OC(=O)c1ccccc1 ; "C26H25N2O12P"] | angl#27 |
| 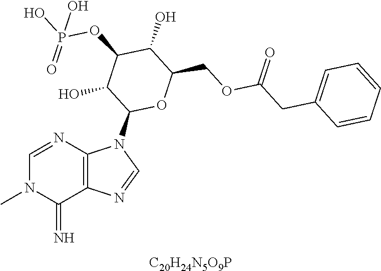 [SMILES: Cn1cnc2c(ncn2[C@@H]2O[C@H](COC(=O)Cc3ccccc3)[C@@H](O)[C@H](OP(=O)(O)O)[C@H]2O)c1=N ; "C20H24N5O9P"] | maglu#11 |
| 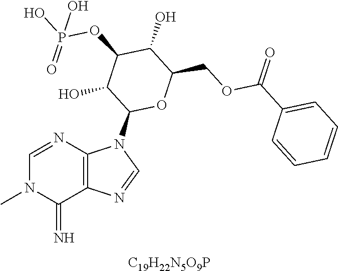 [SMILES: Cn1cnc2c(ncn2[C@@H]2O[C@H](COC(=O)c3ccccc3)[C@@H](O)[C@H](OP(=O)(O)O)[C@H]2O)c1=N ; "C19H22N5O9P"] | maglu#12 |
| 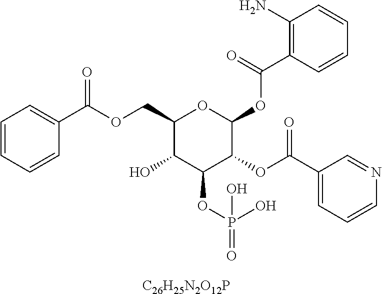 [SMILES: Nc1ccccc1C(=O)O[C@@H]1O[C@H](COC(=O)c2ccccc2)[C@@H](O)[C@H](OP(=O)(O)O)[C@H]1OC(=O)c1cccnc1 ; "C26H25N2O12P"] | angl#28 |
| 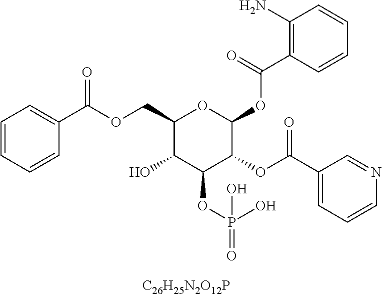 [SMILES: Nc1ccccc1C(=O)O[C@@H]1O[C@H](COC(=O)c2ccccc2)[C@@H](O)[C@H](OP(=O)(O)O)[C@H]1OC(=O)c1cccnc1 ; "C26H25N2O12P"] | angl#29 |
| 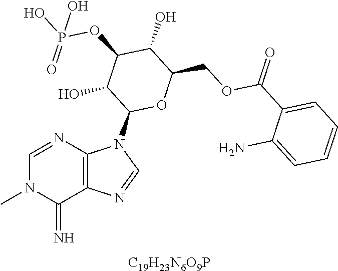 [SMILES: Cn1cnc2c(ncn2[C@@H]2O[C@H](COC(=O)c3ccccc3N)[C@@H](O)[C@H](OP(=O)(O)O)[C@H]2O)c1=N ; "C19H23N6O9P"] | maglu#13 |
| 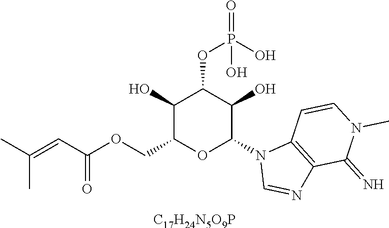 [SMILES: CC(C)=CC(=O)OC[C@H]1O[C@@H](n2cnc3c(=N)n(C)ccc32)[C@H](O)[C@@H](OP(=O)(O)O)[C@@H]1O ; "C17H24N5O9P"] | maglu#14 |
| 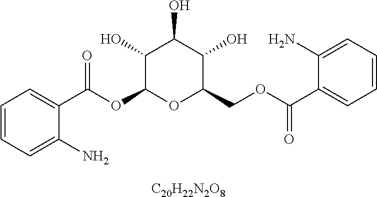 [SMILES: Nc1ccccc1C(=O)OC[C@H]1O[C@@H](OC(=O)c2ccccc2N)[C@H](O)[C@@H](O)[C@@H]1O ; "C20H22N2O8"] | angl#3 |
| 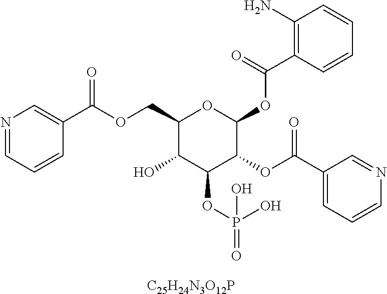 [SMILES: Nc1ccccc1C(=O)O[C@@H]1O[C@H](COC(=O)c2cccnc2)[C@@H](O)[C@H](OP(=O)(O)O)[C@H]1OC(=O)c1cccnc1 ; "C25H24N3O12P"] | angl#30 |
| 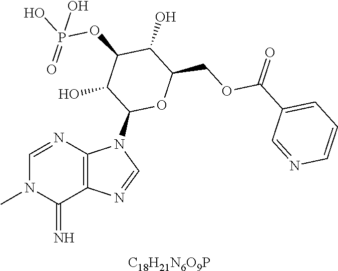 [SMILES: Cn1cnc2c(ncn2[C@@H]2O[C@H](COC(=O)c3cccnc3)[C@@H](O)[C@H](OP(=O)(O)O)[C@H]2O)c1=N ; "C18H21N6O9P"] | maglu#15 |
| 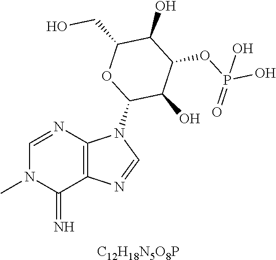 [SMILES: Cn1cnc2c(ncn2[C@@H]2O[C@H](CO)[C@@H](O)[C@H](OP(=O)(O)O)[C@H]2O)c1=N ; "C12H18N5O8P"] | maglu#2 |
| 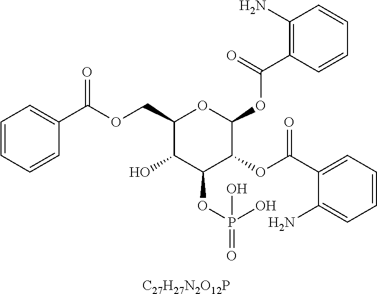 [SMILES: Nc1ccccc1C(=O)O[C@@H]1O[C@H](COC(=O)c2ccccc2)[C@@H](O)[C@H](OP(=O)(O)O)[C@H]1OC(=O)c1ccccc1N ; "C27H27N2O12P"] | angl#32 |
| 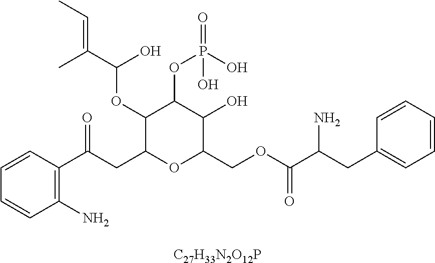 [SMILES: C/C=C(\C)C(O)OC1C(CC(=O)c2ccccc2N)OC(COC(=O)C(N)Cc2ccccc2)C(O)C1OP(=O)(O)O ; "C27H33N2O12P"] | angl#34 |
| 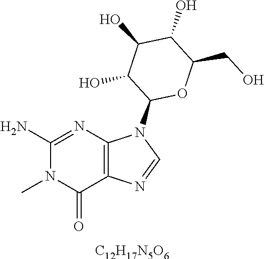 [SMILES: Cn1c(N)nc2c(ncn2[C@@H]2O[C@H](CO)[C@@H](O)[C@H](O)[C@H]2O)c1=O ; "C12H17N5O6"] | mgglu#1 |
| 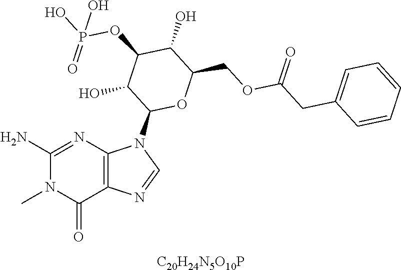 [SMILES: Cn1c(N)nc2c(ncn2[C@@H]2O[C@H](COC(=O)Cc3ccccc3)[C@@H](O)[C@H](OP(=O)(O)O)[C@H]2O)c1=O ; "C20H24N5O10P"] | mgglu#11 |
| 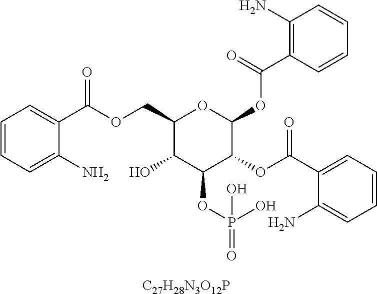 [SMILES: Nc1ccccc1C(=O)OC[C@H]1O[C@@H](OC(=O)c2ccccc2N)[C@H](OC(=O)c2ccccc2N)[C@@H](OP(=O)(O)O)[C@@H]1O ; "C27H28N3O12P"] | angl#36 |
| 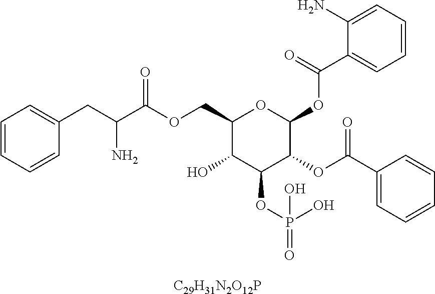 [SMILES: Nc1ccccc1C(=O)O[C@@H]1O[C@H](COC(=O)C(N)Cc2ccccc2)[C@@H](O)[C@H](OP(=O)(O)O)[C@H]1OC(=O)c1ccccc1 ; "C29H31N2O12P"] | angl#38 |
| 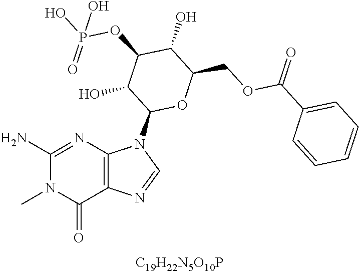 [SMILES: Cn1c(N)nc2c(ncn2[C@@H]2O[C@H](COC(=O)c3ccccc3)[C@@H](O)[C@H](OP(=O)(O)O)[C@H]2O)c1=O ; "C19H22N5O10P"] | mgglu#12 |
| 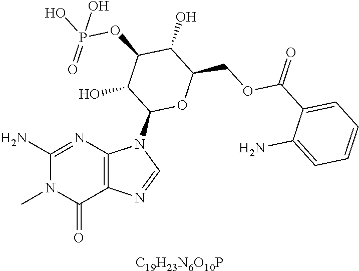 [SMILES: Cn1c(N)nc2c(ncn2[C@@H]2O[C@H](COC(=O)c3ccccc3N)[C@@H](O)[C@H](OP(=O)(O)O)[C@H]2O)c1=O ; "C19H23N6O10P"] | mgglu#13 |
| 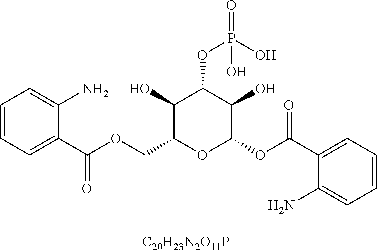 [SMILES: Nc1ccccc1C(=O)OC[C@H]1O[C@@H](OC(=O)c2ccccc2N)[C@H](O)[C@@H](OP(=O)(O)O)[C@@H]1O ; "C20H23N2O11P"] | angl#4 |
| 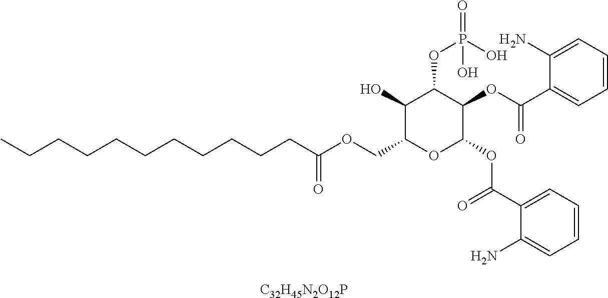 [SMILES: CCCCCCCCCCCC(=O)OC[C@H]1O[C@@H](OC(=O)c2ccccc2N)[C@H](OC(=O)c2ccccc2N)[C@@H](OP(=O)(O)O)[C@@H]1O ; "C32H45N2O12P"] | angl#40 |
| 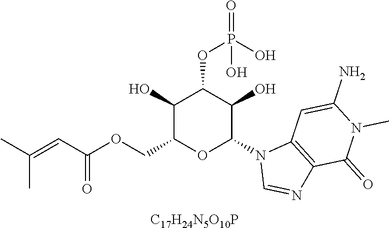 [SMILES: CC(C)=CC(=O)OC[C@H]1O[C@@H](n2cnc3c(=O)n(C)c(N)cc32)[C@H](O)[C@@H](OP(=O)(O)O)[C@@H]1O ; "C17H24N5O10P"] | mgglu#14 |
| 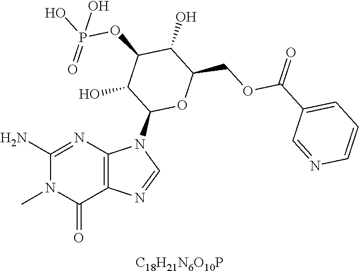 [SMILES: Cn1c(N)nc2c(ncn2[C@@H]2O[C@H](COC(=O)c3cccnc3)[C@@H](O)[C@H](OP(=O)(O)O)[C@H]2O)c1=O ; "C18H21N6O10P"] | mgglu#15 |
| 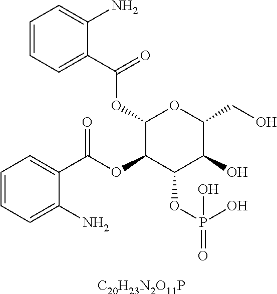 [SMILES: Nc1ccccc1C(=O)O[C@@H]1O[C@H](CO)[C@@H](O)[C@H](OP(=O)(O)O)[C@H]1OC(=O)c1ccccc1N ; "C20H23N2O11P"] | angl#401 |
| 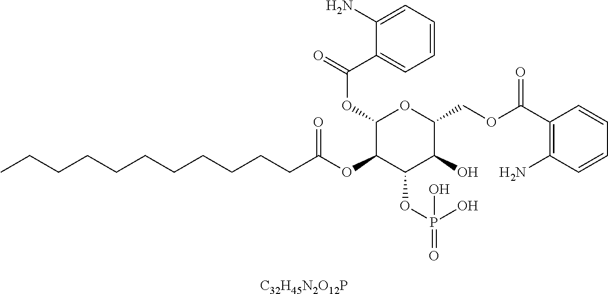 [SMILES: CCCCCCCCCCCC(=O)O[C@H]1[C@H](OC(=O)c2ccccc2N)O[C@H](COC(=O)c2ccccc2N)[C@@H](O)[C@@H]1OP(=O)(O)O ; "C32H45N2O12P"] | angl#41 |
| 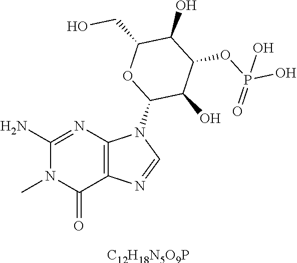 [SMILES: Cn1c(N)nc2c(ncn2[C@@H]2O[C@H](CO)[C@@H](O)[C@H](OP(=O)(O)O)[C@H]2O)c1=O ; "C12H18N5O9P"] | mgglu#2 |
| 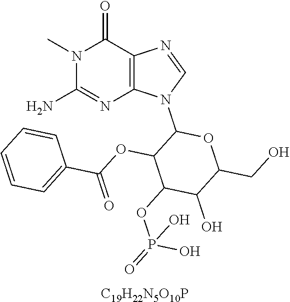 | mgglu#201 |
| 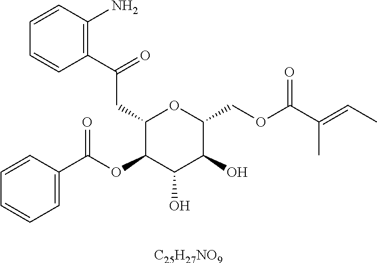 [SMILES: C/C=C(\C)C(=O)OC[C@H]1O[C@@H](CC(=O)c2ccccc2N)[C@H](OC(=O)c2ccccc2)[C@@H](O)[C@@H]1O ; "C25H27NO9"] | angl#42 |
| 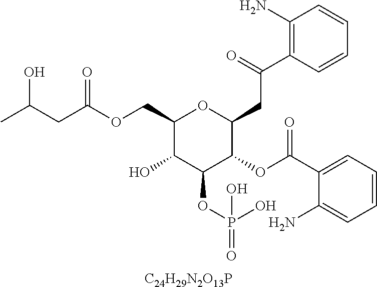 [SMILES: CC(O)CC(=O)OC[C@H]1O[C@@H](CC(=O)c2ccccc2N)[C@H](OC(=O)c2ccccc2N)[C@@H](OP(=O)(O)O)[C@@H]1O ; "C24H29N2O13P"] | angl#44 |
| 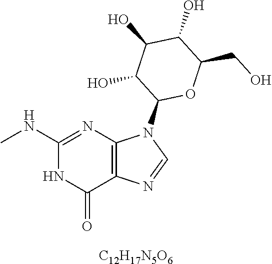 [SMILES: CNc1nc2c(ncn2[C@@H]2O[C@H](CO)[C@@H](O)[C@H](O)[C@H]2O)c(=O)[nH]1 ; "C12H17N5O6"] | mgglu#3 |
| 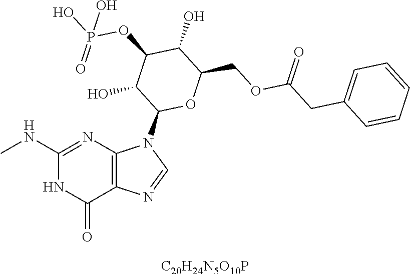 [SMILES: CNc1nc2c(ncn2[C@@H]2O[C@H](COC(=O)Cc3ccccc3)[C@@H](O)[C@H](OP(=O)(O)O)[C@H]2O)c(=O)[nH]1 ; "C20H24N5O10P"] | mgglu#31 |
| 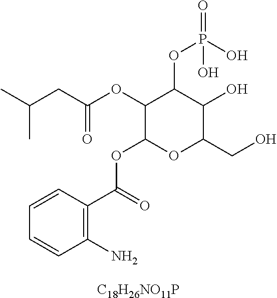 | angl#46 |
| 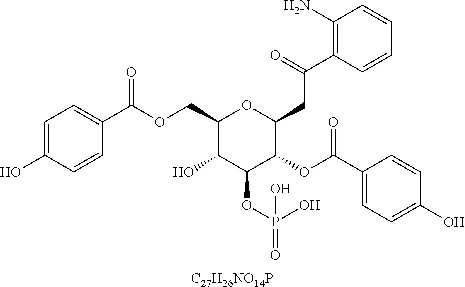 [SMILES: Nc1ccccc1C(=O)C[C@@H]1O[C@H](COC(=O)c2ccc(O)cc2)[C@@H](O)[C@H](OP(=O)(O)O)[C@H]1OC(=O)c1ccc(O)cc1 ; "C27H26NO14P"] | angl#47 |
| 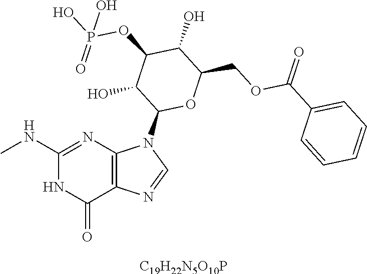 [SMILES: CNc1nc2c(ncn2[C@@H]2O[C@H](COC(=O)c3ccccc3)[C@@H](O)[C@H](OP(=O)(O)O)[C@H]2O)c(=O)[nH]1 ; "C19H22N5O10P"] | mgglu#32 |
| 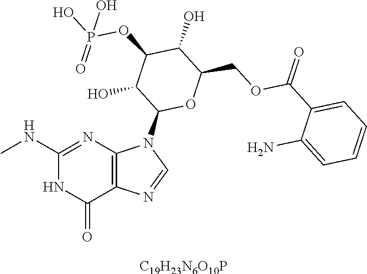 [SMILES: CNc1nc2c(ncn2[C@@H]2O[C@H](COC(=O)c3ccccc3N)[C@@H](O)[C@H](OP(=O)(O)O)[C@H]2O)c(=O)[nH]1 ; "C19H23N6O10P"] | mgglu#33 |
| 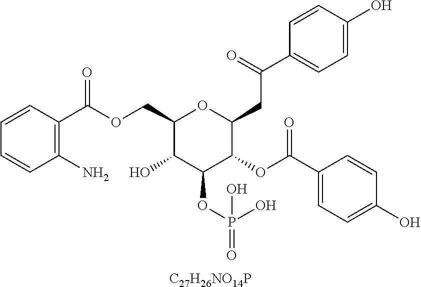 [SMILES: Nc1ccccc1C(=O)OC[C@H]1O[C@@H](CC(=O)c2ccc(O)cc2)[C@H](OC(=O)c2ccc(O)cc2)[C@@H](OP(=O)(O)O)[C@@H]1O ; "C27H26NO14P"] | angl#48 |
| 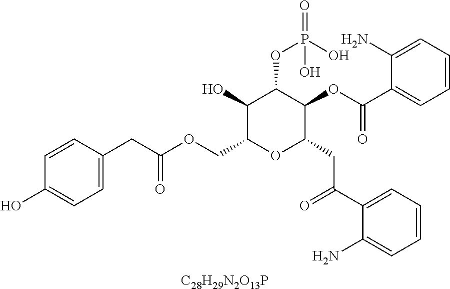 [SMILES: Nc1ccccc1C(=O)C[C@@H]1O[C@H](COC(=O)Cc2ccc(O)cc2)[C@@H](O)[C@H](OP(=O)(O)O)[C@H]1OC(=O)c1ccccc1N ; "C28H29N2O13P"] | angl#50 |
| 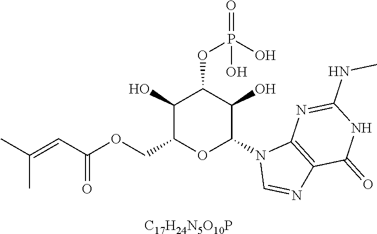 [SMILES: CNc1nc2c(ncn2[C@@H]2O[C@H](COC(=O)C=C(C)C)[C@@H](O)[C@H](OP(=O)(O)O)[C@H]2O)c(=O)[nH]1 ; "C17H24N5O10P"] | mgglu#34 |
| 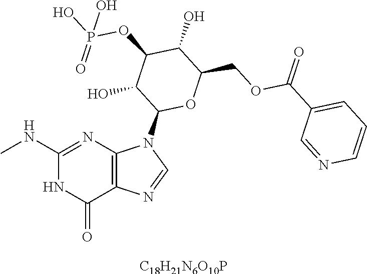 [SMILES: CNc1nc2c(ncn2[C@@H]2O[C@H](COC(=O)c3cccnc3)[C@@H](O)[C@H](OP(=O)(O)O)[C@H]2O)c(=O)[nH]1 ; "C18H21N6O10P"] | mgglu#35 |
| 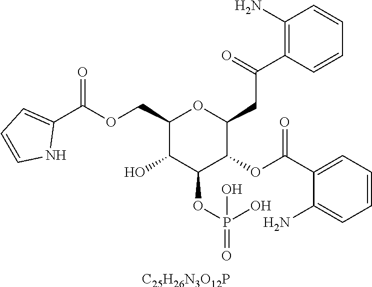 [SMILES: Nc1ccccc1C(=O)C[C@@H]1O[C@H](COC(=O)c2ccc[nH]2)[C@@H](O)[C@H](OP(=O)(O)O)[C@H]1OC(=O)c1ccccc1N ; "C25H26N3O12P"] | angl#51 |
| 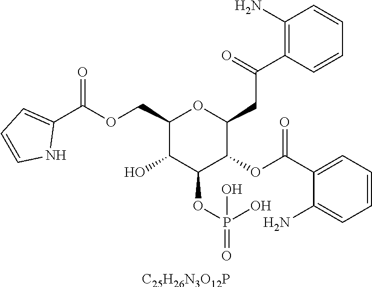 [SMILES: Nc1ccccc1C(=O)C[C@@H]1O[C@H](COC(=O)c2ccc[nH]2)[C@@H](O)[C@H](OP(=O)(O)O)[C@H]1OC(=O)c1ccccc1N ; "C25H26N3O12P"] | angl#52 |
| 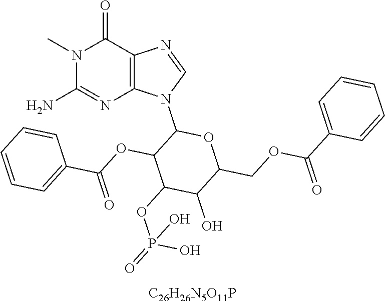 | mgglu#4 |
| 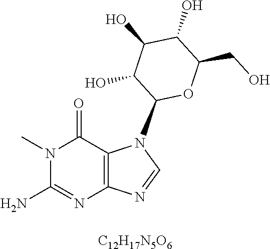 [SMILES: Cn1c(N)nc2ncn([C@@H]3O[C@H](CO)[C@@H](O)[C@H](O)[C@H]3O)c2c1=O ; "C12H17N5O6"] | mgglu#5 |
| 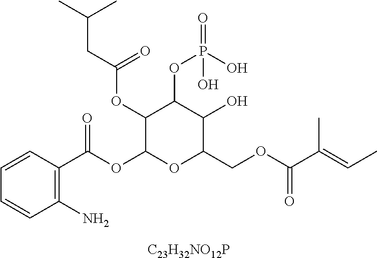 [SMILES: C/C=C(\C)C(=O)OCC1OC(OC(=O)c2ccccc2N)C(OC(=O)CC(C)C)C(OP(=O)(O)O)C1O ; "C23H32NO12P"] | angl#54 |
| 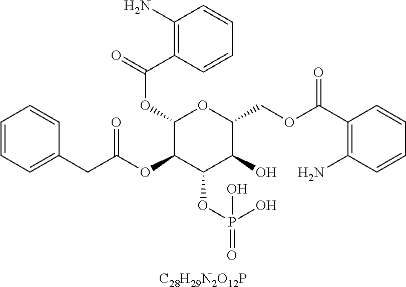 [SMILES: Nc1ccccc1C(=O)OC[C@H]1O[C@@H](OC(=O)c2ccccc2N)[C@H](OC(=O)Cc2ccccc2)[C@@H](OP(=O)(O)O)[C@@H]1O ; "C28H29N2O12P"] | angl#56 |
| 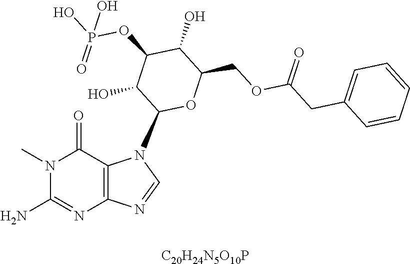 [SMILES: Cn1c(N)nc2ncn([C@@H]3O[C@H](COC(=O)Cc4ccccc4)[C@@H](O)[C@H](OP(=O)(O)O)[C@H]3O)c2c1=O ; "C20H24N5O10P"] | mgglu#51 |
| 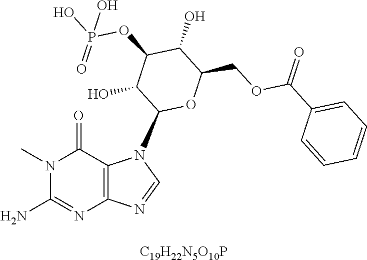 [SMILES: Cn1c(N)nc2ncn([C@@H]3O[C@H](COC(=O)c4ccccc4)[C@@H](O)[C@H](OP(=O)(O)O)[C@H]3O)c2c1=O ; "C19H22N5O10P"] | mgglu#52 |
| 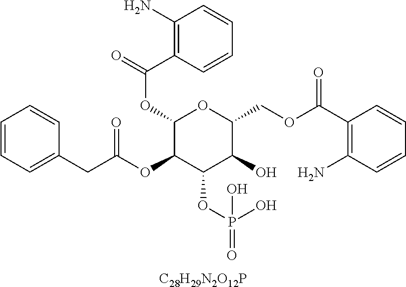 [SMILES: Nc1ccccc1C(=O)OC[C@H]1O[C@@H](OC(=O)c2ccccc2N)[C@H](OC(=O)Cc2ccccc2)[C@@H](OP(=O)(O)O)[C@@H]1O ; "C28H29N2O12P"] | angl#56 |
| 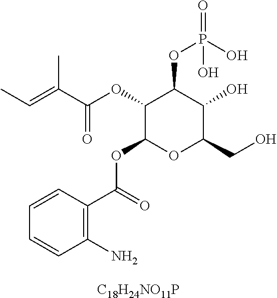 | angl#601 |
| 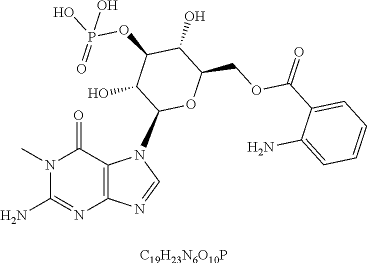 [SMILES: Cn1c(N)nc2ncn([C@@H]3O[C@H](COC(=O)c4ccccc4N)[C@@H](O)[C@H](OP(=O)(O)O)[C@H]3O)c2c1=O ; "C19H23N6O10P"] | mgglu#53 |
| 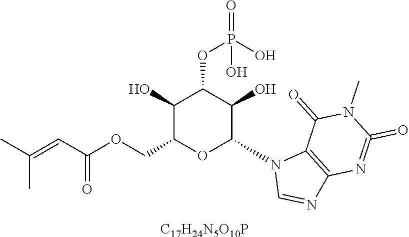 | mgglu#54 |
| 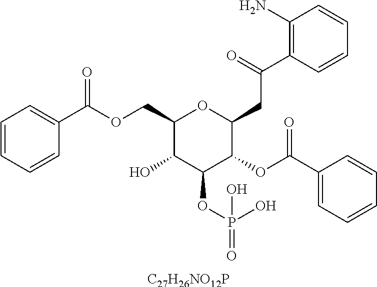 [SMILES: Nc1ccccc1C(=O)C[C@@H]1O[C@H](COC(=O)c2ccccc2)[C@@H](O)[C@H](OP(=O)(O)O)[C@H]1OC(=O)c1ccccc1 ; "C27H26NO12P"] | angl#70 |
| 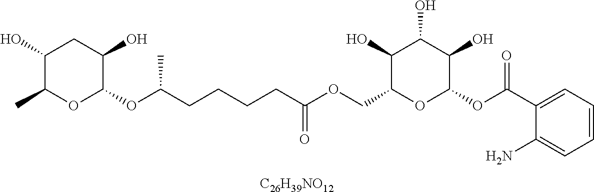 [SMILES: C[C@H](CCCCC(=O)OC[C@H]1O[C@@H](OC(=O)c2ccccc2N)[C@H](O)[C@@H](O)[C@@H]1O)O[C@@H]1O[C@@H](C)[C@H](O)C[C@H]1O ; "C26H39NO12"] | anglas#1 |
| 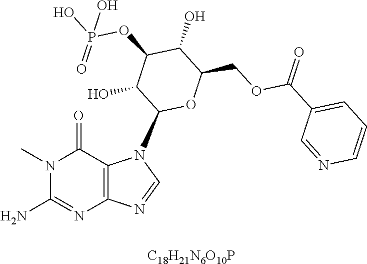 [SMILES: Cn1c(N)nc2ncn([C@@H]3O[C@H](COC(=O)c4cccnc4)[C@@H](O)[C@H](OP(=O)(O)O)[C@H]3O)c2c1=O ; "C18H21N6O10P"] | mgglu#55 |
| 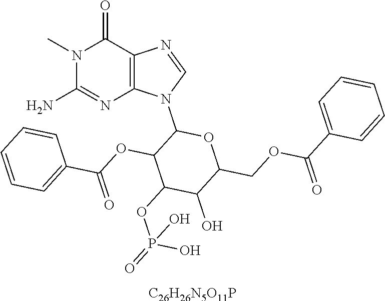 | mgglu#6 |
| 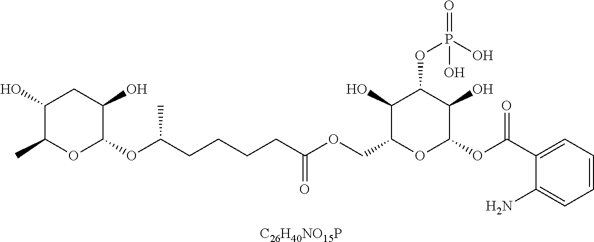 [SMILES: C[C@H](CCCCC(=O)OC[C@H]1O[C@@H](OC(=O)c2ccccc2N)[C@H](O)[C@@H](OP(=O)(O)O)[C@@H]1O)O[C@@H]1O[C@@H](C)[C@H](O)C[C@H]1O ; "C26H40NO15P"] | anglas#2 |
| 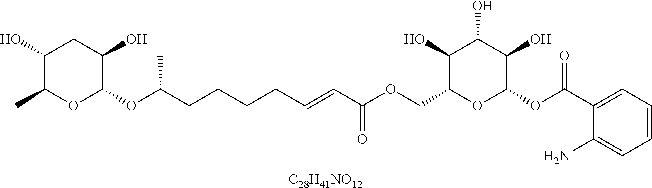 [SMILES: C[C@H](CCCC/C=C/C(=O)OC[C@H]1O[C@@H](OC(=O)c2ccccc2N)[C@H](O)[C@@H](O)[C@@H]1O)O[C@@H]1O[C@@H](C)[C@H](O)C[C@H]1O ; "C28H41NO12"] | anglas#3 |
| 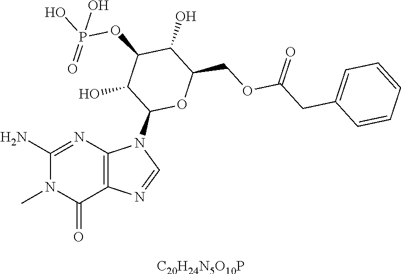 [SMILES: Cn1c(N)nc2c(ncn2[C@@H]2O[C@H](COC(=O)Cc3ccccc3)[C@@H](O)[C@H](OP(=O)(O)O)[C@H]2O)c1=O ; "C20H24N5O10P"] | mgglu#7 |
| 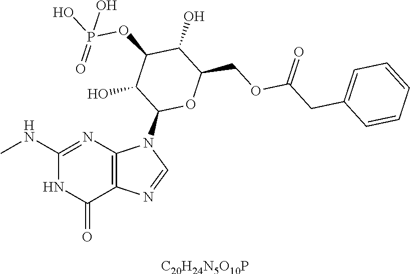 [SMILES: CNc1nc2c(ncn2[C@@H]2O[C@H](COC(=O)Cc3ccccc3)[C@@H](O)[C@H](OP(=O)(O)O)[C@H]2O)c(=O)[nH]1 ; "C20H24N5O10P"] | mgglu#8 |
| 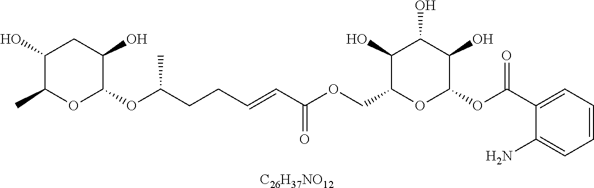 [SMILES: C[C@H](CC/C=C/C(=O)OC[C@H]1O[C@@H](OC(=O)c2ccccc2N)[C@H](O)[C@@H](O)[C@@H]1O)O[C@@H]1O[C@@H](C)[C@H](O)C[C@H]1O ; "C26H37NO12"] | anglas#7 |
| 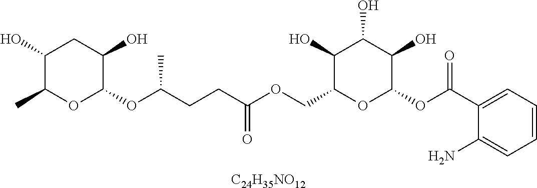 [SMILES: C[C@H](CCC(=O)OC[C@H]1O[C@@H](OC(=O)c2ccccc2N)[C@H](O)[C@@H](O)[C@@H]1O)O[C@@H]1O[C@@H](C)[C@H](O)C[C@H]1O ; "C24H35NO12"] | anglas#9 |
| 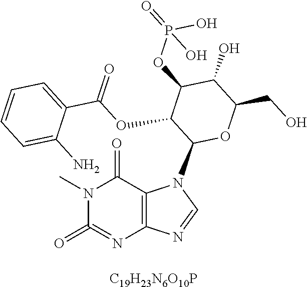 | mglu#503 |
| 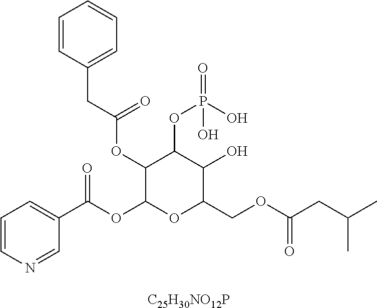 [SMILES: CC(C)CC(=O)OCC1OC(OC(=O)c2cccnc2)C(OC(=O)Cc2ccccc2)C(OP(=O)(O)O)C1O ; "C25H30NO12P"] | nglu#10 |
| 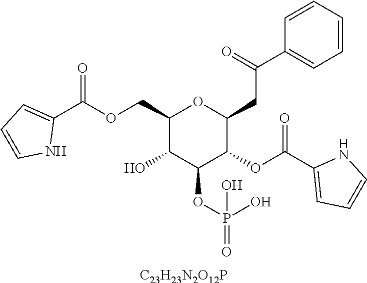 [SMILES: O=C(C[C@@H]1O[C@H](COC(=O)c2ccc[nH]2)[C@@H](O)[C@H](OP(=O)(O)O)[C@H]1OC(=O)c1ccc[nH]1)c1ccccc1 ; "C23H23N2O12P"] | bzglu#10 |
| 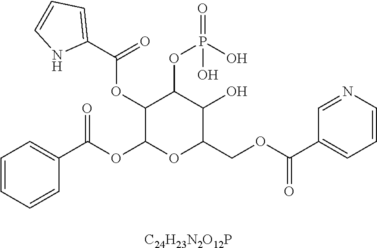 [SMILES: O=C(OCC1OC(OC(=O)c2ccccc2)C(OC(=O)c2ccc[nH]2)C(OP(=O)(O)O)C1O)c1cccnc1 ; "C24H23N2O12P"] | bzglu#12 |
| 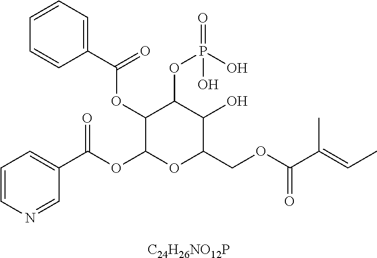 [SMILES: C/C=C(\C)C(=O)OCC1OC(OC(=O)c2cccnc2)C(OC(=O)c2ccccc2)C(OP(=O)(O)O)C1O ; "C24H26NO12P"] | nglu#3 |
| 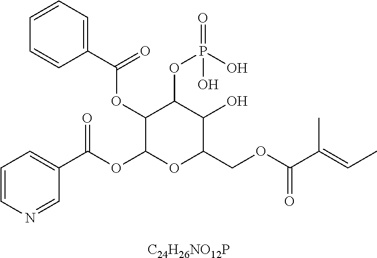 [SMILES: C/C=C(\C)C(=O)OCC1OC(OC(=O)c2cccnc2)C(OC(=O)c2ccccc2)C(OP(=O)(O)O)C1O ; "C24H26NO12P"] | nglu#4 |
| 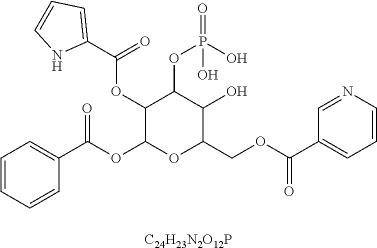 [SMILES: O=C(OCC1OC(OC(=O)c2ccccc2)C(OC(=O)c2ccc[nH]2)C(OP(=O)(O)O)C1O)c1cccnc1 ; "C24H23N2O12P"] | bzglu#13 |
| 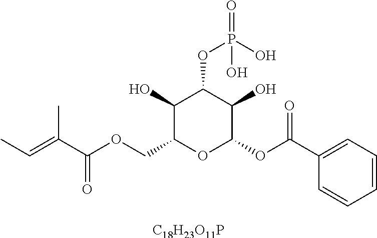 [SMILES: C/C=C(\C)C(=O)OC[C@H]1O[C@@H](OC(=O)c2ccccc2)[C@H](O)[C@@H](OP(=O)(O)O)[C@@H]1O ; "C18H23O11P"] | bzglu#201 |
| 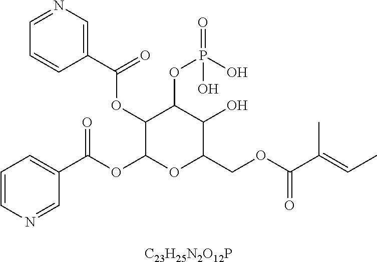 [SMILES: C/C=C(\C)C(=O)OCC1OC(OC(=O)c2cccnc2)C(OC(=O)c2cccnc2)C(OP(=O)(O)O)C1O ; "C23H25N2O12P"] | nglu#5 |
| 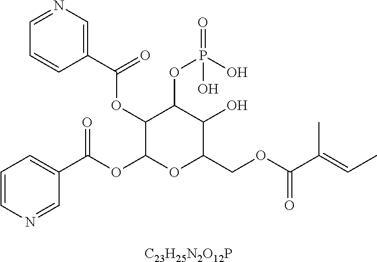 [SMILES: C/C=C(\C)C(=O)OCC1OC(OC(=O)c2cccnc2)C(OC(=O)c2cccnc2)C(OP(=O)(O)O)C1O ; "C23H25N2O12P"] | nglu#6 |
| 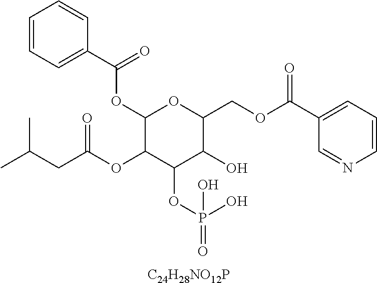 [SMILES: CC(C)CC(=O)OC1C(OC(=O)c2ccccc2)OC(COC(=O)c2cccnc2)C(O)C1OP(=O)(O)O ; "C24H28NO12P"] | bzglu#4 |
| 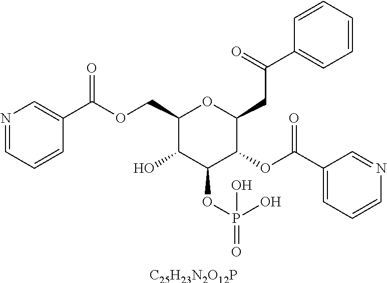 [SMILES: O=C(C[C@@H]1O[C@H](COC(=O)c2cccnc2)[C@@H](O)[C@H](OP(=O)(O)O)[C@H]1OC(=O)c1cccnc1)c1ccccc1 ; "C25H23N2O12P"] | bzglu#6 |
| 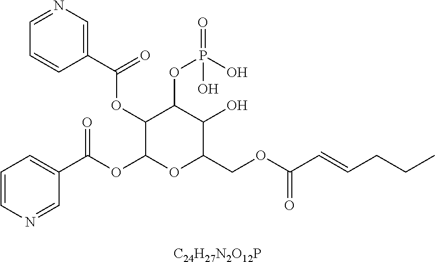 [SMILES: CCC/C=C/C(=O)OCC1OC(OC(=O)c2cccnc2)C(OC(=O)c2cccnc2)C(OP(=O)(O)O)C1O ; "C24H27N2O12P"] | nglu#7 |
| 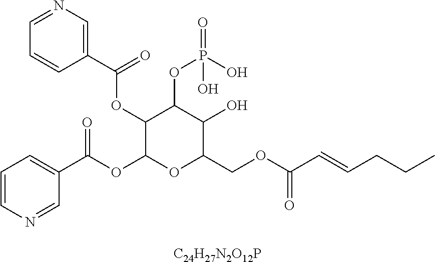 [SMILES: CCC/C=C/C(=O)OCC1OC(OC(=O)c2cccnc2)C(OC(=O)c2cccnc2)C(OP(=O)(O)O)C1O ; "C24H27N2O12P"] | nglu#8 |
| 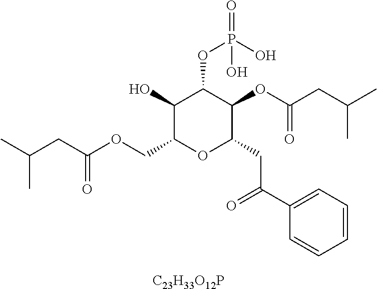 [SMILES: CC(C)CC(=O)OC[C@H]1O[C@@H](CC(=O)c2ccccc2)[C@H](OC(=O)CC(C)C)[C@@H](OP(=O)(O)O)[C@@H]1O ; "C23H33O12P"] | bzglu#8 |
| 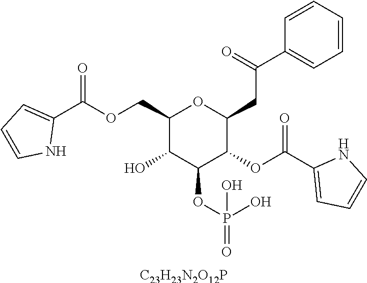 [SMILES: O=C(C[C@@H]1O[C@H](COC(=O)c2ccc[nH]2)[C@@H](O)[C@H](OP(=O)(O)O)[C@H]1OC(=O)c1ccc[nH]1)c1ccccc1 ; "C23H23N2O12P"] | bzglu#9 |
| 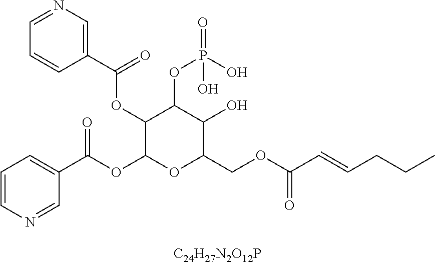 [SMILES: CCC/C=C/C(=O)OCC1OC(OC(=O)c2cccnc2)C(OC(=O)c2cccnc2)C(OP(=O)(O)O)C1O ; "C24H27N2O12P"] | nglu#9 |
| 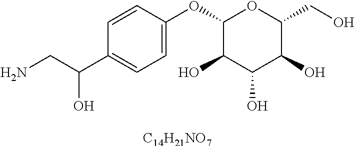 [SMILES: NCC(O)c1ccc(O[C@@H]2O[C@H](CO)[C@@H](O)[C@H](O)[C@H]2O)cc1 ; "C14H21NO7"] | oglu#1 |
| 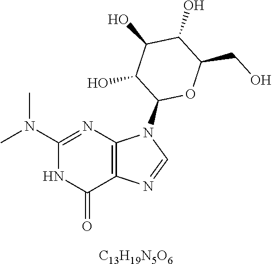 [SMILES: CN(C)c1nc2c(ncn2[C@@H]2O[C@H](CO)[C@@H](O)[C@H](O)[C@H]2O)c(=O)[nH]1 ; "C13H19N5O6"] | dmgglu#1 |
| 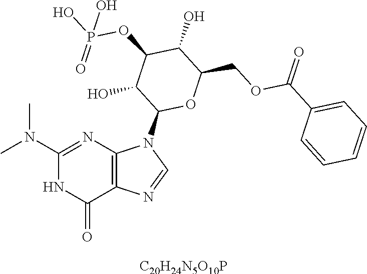 [SMILES: CN(C)c1nc2c(ncn2[C@@H]2O[C@H](COC(=O)c3ccccc3)[C@@H](O)[C@H](OP(=O)(O)O)[C@H]2O)c(=O)[nH]1 ; "C20H24N5O10P"] | dmgglu#12 |
| 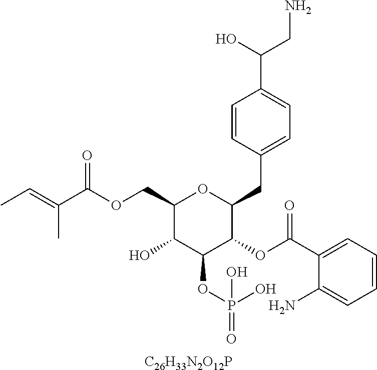 [SMILES: C/C=C(\C)C(=O)OC[C@H]1O[C@@H](Cc2ccc(C(O)CN)cc2)[C@H](OC(=O)c2ccccc2N)[C@@H](OP(=O)(O)O)[C@@H]1O ; "C26H33N2O12P"] | oglu#10 |
| 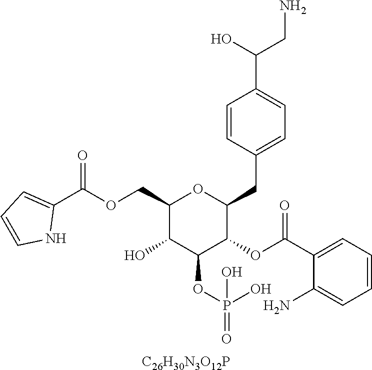 [SMILES: NCC(O)c1ccc(C[C@@H]2O[C@H](COC(=O)c3ccc[nH]3)[C@@H](O)[C@H](OP(=O)(O)O)[C@H]2OC(=O)c2ccccc2N)cc1 ; "C26H30N3O12P"] | oglu#1401 |
| 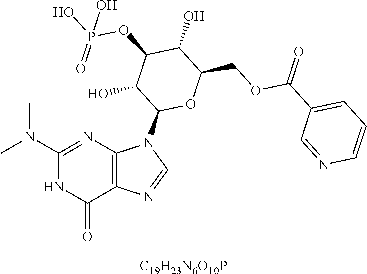 [SMILES: CN(C)c1nc2c(ncn2[C@@H]2O[C@H](COC(=O)c3cccnc3)[C@@H](O)[C@H](OP(=O)(O)O)[C@H]2O)c(=O)[nH]1 ; "C19H23N6O10P"] | dmgglu#15 |
| 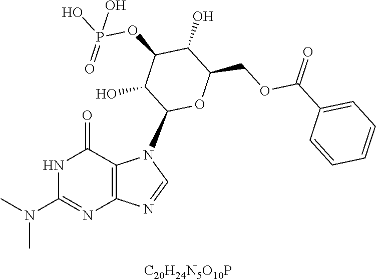 [SMILES: CN(C)c1nc2ncn([C@@H]3O[C@H](COC(=O)c4ccccc4)[C@@H](O)[C@H](OP(=O)(O)O)[C@H]3O)c2c(=O)[nH]1 ; "C20H24N5O10P"] | dmgglu#32 |
| 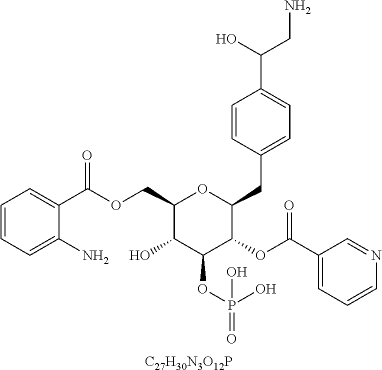 [SMILES: NCC(O)c1ccc(C[C@@H]2O[C@H](COC(=O)c3ccccc3N)[C@@H](O)[C@H](OP(=O)(O)O)[C@H]2OC(=O)c2cccnc2)cc1 ; "C27H30N3O12P"] | oglu#15 |
| 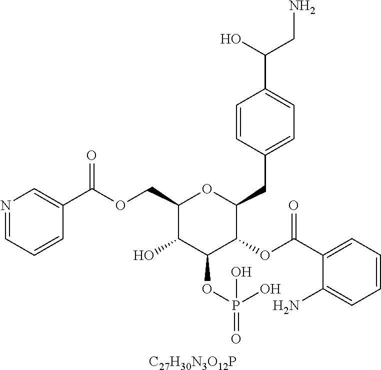 [SMILES: NCC(O)c1ccc(C[C@@H]2O[C@H](COC(=O)c3cccnc3)[C@@H](O)[C@H](OP(=O)(O)O)[C@H]2OC(=O)c2ccccc2N)cc1 ; "C27H30N3O12P"] | oglu#16 |
| 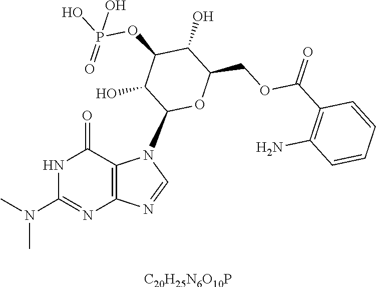 [SMILES: CN(C)c1nc2ncn([C@@H]3O[C@H](COC(=O)c4ccccc4N)[C@@H](O)[C@H](OP(=O)(O)O)[C@H]3O)c2c(=O)[nH]1 ; "C20H25N6O10P"] | dmgglu#33 |
| 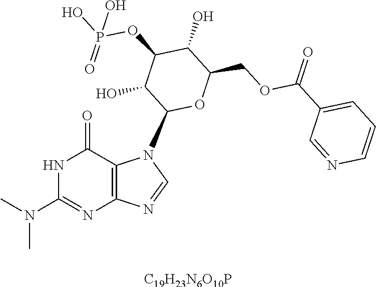 [SMILES: CN(C)c1nc2ncn([C@@H]3O[C@H](COC(=O)c4cccnc4)[C@@H](O)[C@H](OP(=O)(O)O)[C@H]3O)c2c(=O)[nH]1 ; "C19H23N6O10P"] | dmgglu#35 |
| 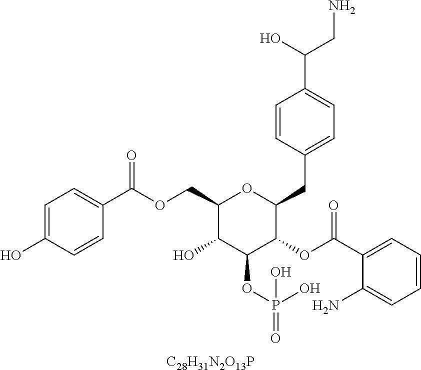 [SMILES: NCC(O)c1ccc(C[C@@H]2O[C@H](COC(=O)c3ccc(O)cc3)[C@@H](O)[C@H](OP(=O)(O)O)[C@H]2OC(=O)c2ccccc2N)cc1 ; "C28H31N2O13P"] | oglu#17 |
| 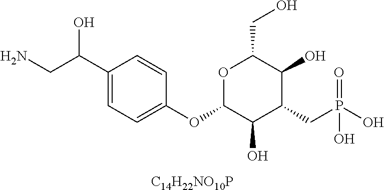 [SMILES: NCC(O)c1ccc(O[C@@H]2O[C@H](CO)[C@@H](O)[C@H](CP(=O)(O)O)[C@H]2O)cc1 ; "C14H22NO10P"] | oglu#2 |
| 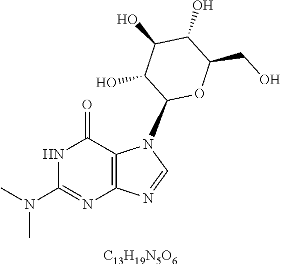 [SMILES: CN(C)c1nc2ncn([C@@H]3O[C@H](CO)[C@@H](O)[C@H](O)[C@H]3O)c2c(=O)[nH]1 ; "C13H19N5O6"] | dmglu#3 |
| 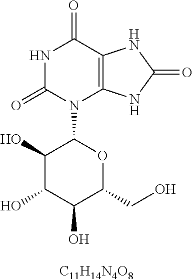 | gluric#1 |
| 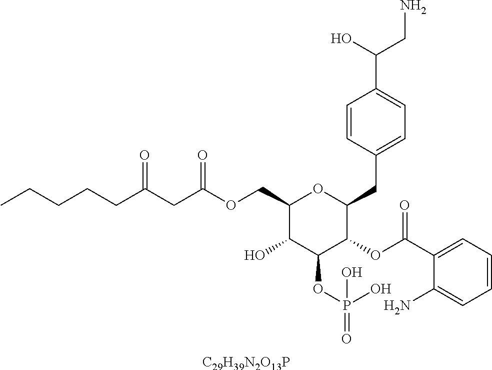 [SMILES: CCCCCC(=O)CC(=O)OC[C@H]1O[C@@H](Cc2ccc(C(O)CN)cc2)[C@H](OC(=O)c2ccccc2N)[C@@H](OP(=O)(O)O)[C@@H]1O ; "C29H39N2O13P"] | oglu#22 |
| 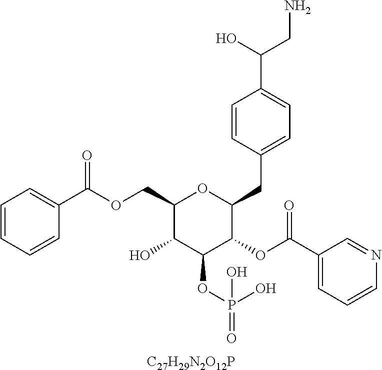 [SMILES: NCC(O)c1ccc(C[C@@H]2O[C@H](COC(=O)c3ccccc3)[C@@H](O)[C@H](OP(=O)(O)O)[C@H]2OC(=O)c2cccnc2)cc1 ; "C27H29N2O12P"] | oglu#24 |
| 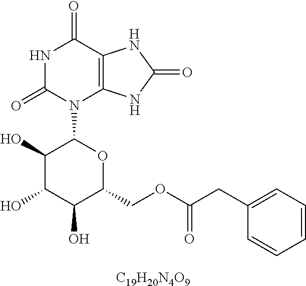 [SMILES: O=C(Cc1ccccc1)OC[C@H]1O[C@@H](n2c(=O)[nH]c(=O)c3[nH]c(=O)[nH]c32)[C@H](O)[C@@H](O)[C@@H]1O ; "C19H20N4O9"] | gluric#3 |
| 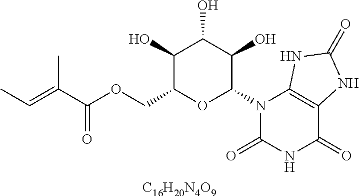 [SMILES: C/C=C(\C)C(=O)OC[C@H]1O[C@@H](n2c(=O)[nH]c(=O)c3[nH]c(=O)[nH]c32)[C@H](O)[C@@H](O)[C@@H]1O ; "C16H20N4O9"] | gluric#5 |
| 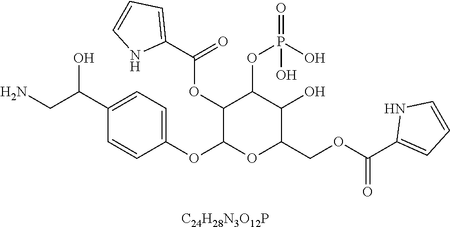 [SMILES: NCC(O)c1ccc(OC2OC(COC(=O)c3ccc[nH]3)C(O)C(OP(=O)(O)O)C2OC(=O)c2ccc[nH]2)cc1 ; "C24H28N3O12P"] | oglu#26 |
| 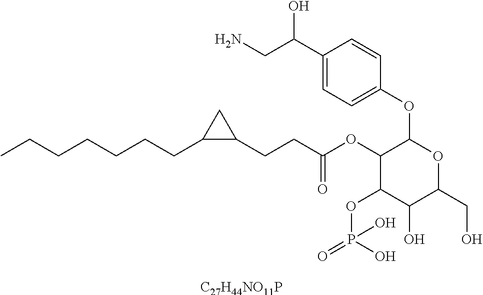 [SMILES: CCCCCCCC1CC1CCC(=O)OC1C(Oc2ccc(C(O)CN)cc2)OC(CO)C(O)C1OP(=O)(O)O ; "C27H44NO11P"] | oglu#28 |
| 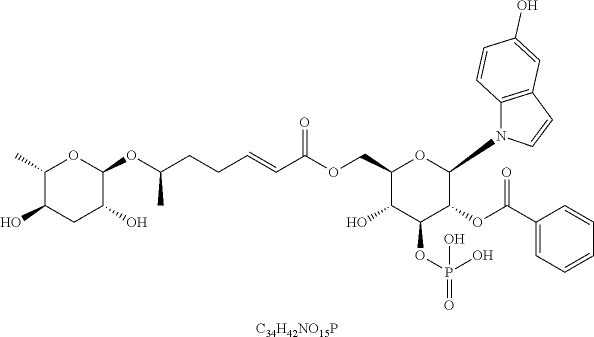 [SMILES: C[C@H](CC/C=C/C(=O)OC[C@H]1O[C@@H](n2ccc3cc(O)ccc32)[C@H](OC(=O)c2ccccc2)[C@@H](OP(=O)(O)O)[C@@H]1O)O[C@@H]1O[C@@H](C)[C@H](O)C[C@H]1O ; "C34H42NO15P"] | higlas#7 |
| 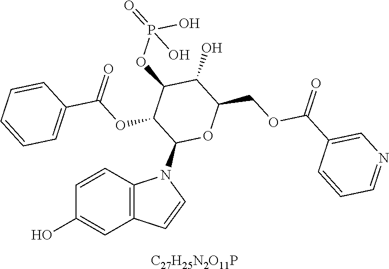 [SMILES: O=C(OC[C@H]1O[C@@H](n2ccc3cc(O)ccc32)[C@H](OC(=O)c2ccccc2)[C@@H](OP(=O)(O)O)[C@@H]1O)c1cccnc1 ; "C27H25N2O11P"] | higlu#3 |
| 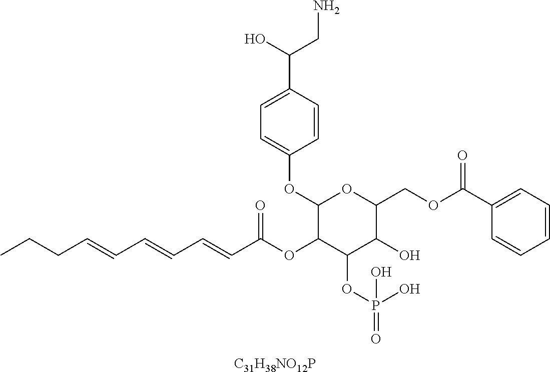 [SMILES: CCC/C=C/C=C/C=C/C(=O)OC1C(Oc2ccc(C(O)CN)cc2)OC(COC(=O)c2ccccc2)C(O)C1OP(=O)(O)O ; "C31H38NO12P"] | oglu#30 |
| 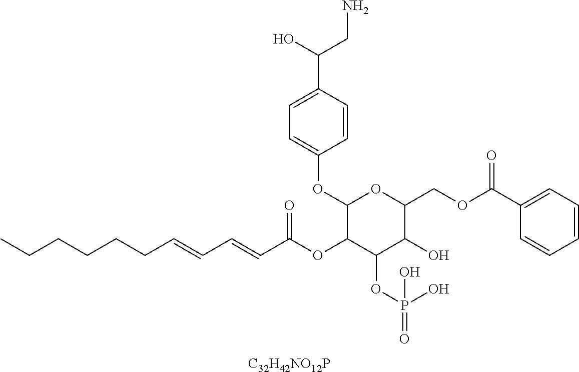 [SMILES: CCCCCC/C=C/C=C/C(=O)OC1C(Oc2ccc(C(O)CN)cc2)OC(COC(=O)c2ccccc2)C(O)C1OP(=O)(O)O ; "C32H42NO12P"] | oglu#32 |
| 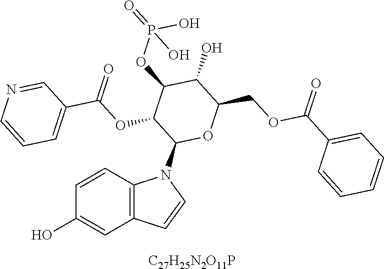 [SMILES: O=C(OC[C@H]1O[C@@H](n2ccc3cc(O)ccc32)[C@H](OC(=O)c2cccnc2)[C@@H](OP(=O)(O)O)[C@@H]1O)c1ccccc1 ; "C27H25N2O11P"] | higlu#4 |
| 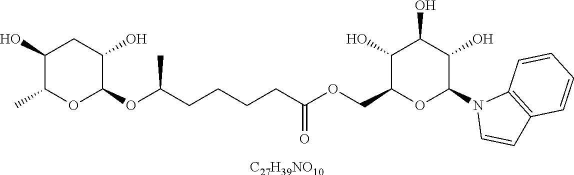 [SMILES: C[C@@H](CCCCC(=O)OC[C@@H]1O[C@H](n2ccc3ccccc32)[C@@H](O)[C@H](O)[C@H]1O)O[C@H]1O[C@H](C)[C@@H](O)C[C@@H]1O ; "C27H39NO10"] | iglas#1 |
| 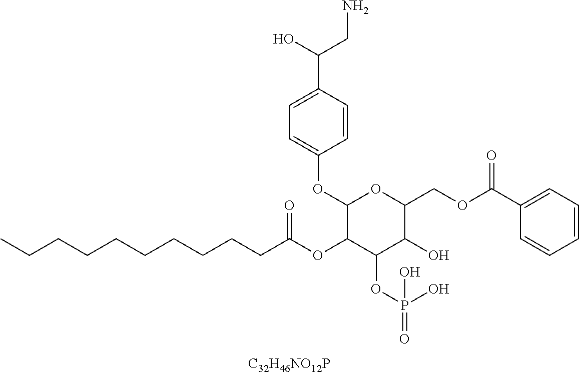 [SMILES: CCCCCCCCCCC(=O)OC1C(Oc2ccc(C(O)CN)cc2)OC(COC(=O)c2ccccc2)C(O)C1OP(=O)(O)O ; "C32H46NO12P"] | oglu#34 |
| 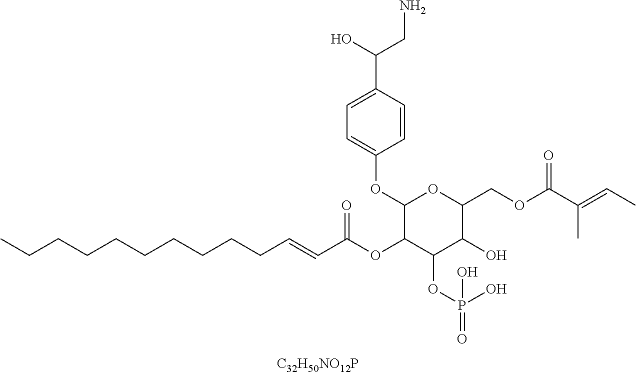 [SMILES: C/C=C(\C)C(=O)OCC1OC(Oc2ccc(C(O)CN)cc2)C(OC(=O)/C=C/CCCCCCCCCC)C(OP(=O)(O)O)C1O ; "C32H50NO12P"] | oglu#36 |
| 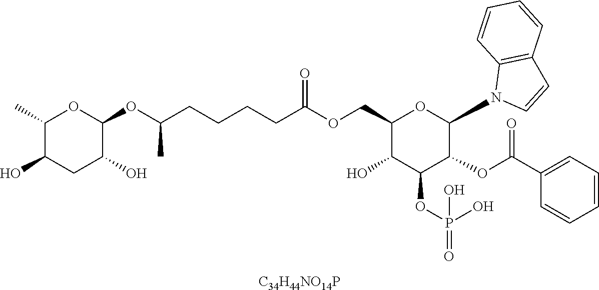 [SMILES: C[C@H](CCCCC(=O)OC[C@H]1O[C@@H](n2ccc3ccccc32)[C@H](OC(=O)c2ccccc2)[C@@H](OP(=O)(O)O)[C@@H]1O)O[C@@H]1O[C@@H](C)[C@H](O)C[C@H]1O ; "C34H44NO14P"] | iglas#101 |
| 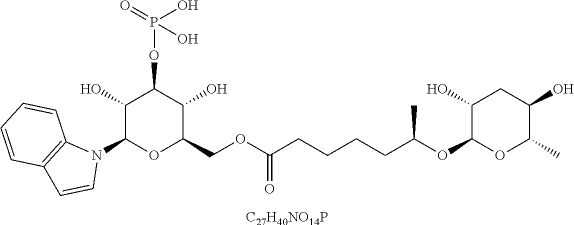 | iglas#2 |
| 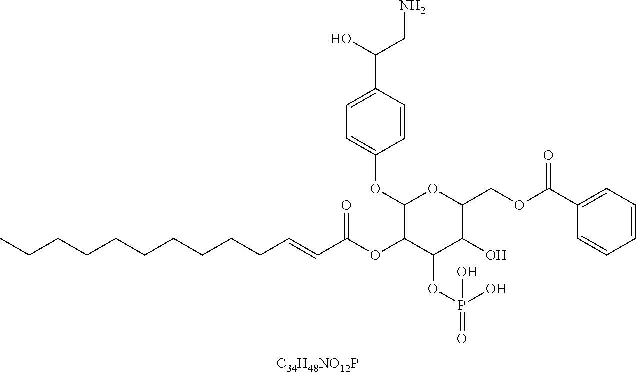 [SMILES: CCCCCCCCCC/C=C/C(=O)OC1C(Oc2ccc(C(O)CN)cc2)OC(COC(=O)c2ccccc2)C(O)C1OP(=O)(O)O ; "C34H48NO12P"] | oglu#38 |
| 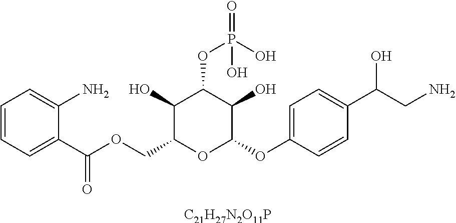 [SMILES: NCC(O)c1ccc(O[C@@H]2O[C@H](COC(=O)c3ccccc3N)[C@@H](O)[C@H](OP(=O)(O)O)[C@H]2O)cc1 ; "C21H27N2O11P"] | oglu#4 |
| 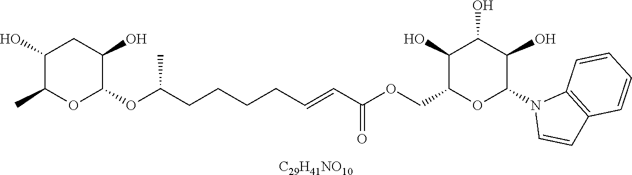 [SMILES: C[C@H](CCCC/C=C/C(=O)OC[C@H]1O[C@@H](n2ccc3ccccc32)[C@H](O)[C@@H](O)[C@@H]1O)O[C@@H]1O[C@@H](C)[C@H](O)C[C@H]1O ; "C29H41NO10"] | iglas#3 |
| 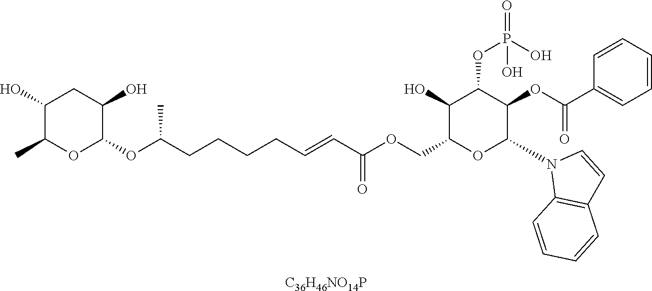 [SMILES: C[C@H](CCCC/C=C/C(=O)OC[C@H]1O[C@@H](n2ccc3ccccc32)[C@H](OC(=O)c2ccccc2)[C@@H](OP(=O)(O)O)[C@@H]1O)O[C@@H]1O[C@@H](C)[C@H](O)C[C@H]1O ; "C36H46NO14P"] | iglas#301 |
| 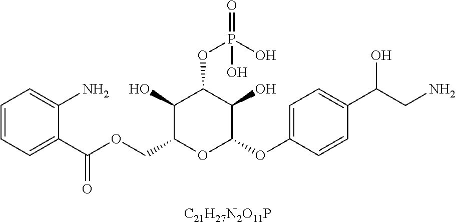 [SMILES: NCC(O)c1ccc(O[C@@H]2O[C@H](COC(=O)c3ccccc3N)[C@@H](O)[C@H](OP(=O)(O)O)[C@H]2O)cc1 ; "C21H27N2O11P"] | oglu#4 |
| 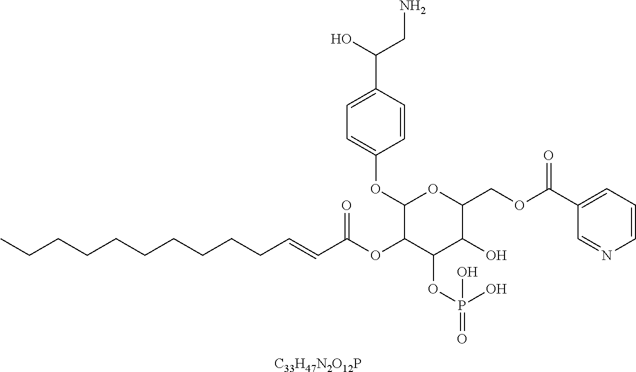 [SMILES: CCCCCCCCCC/C=C/C(=O)OC1C(Oc2ccc(C(O)CN)cc2)OC(COC(=O)c2cccnc2)C(O)C1OP(=O)(O)O ; "C33H47N2O12P"] | oglu#40 |
| 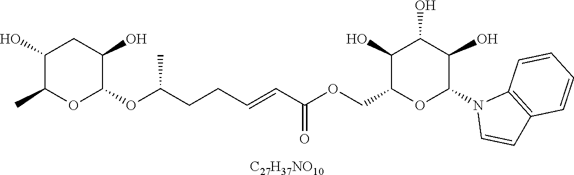 [SMILES: C[C@H](CC/C=C/C(=O)OC[C@H]1O[C@@H](n2ccc3ccccc32)[C@H](O)[C@@H](O)[C@@H]1O)O[C@@H]1O[C@@H](C)[C@H](O)C[C@H]1O ; "C27H37NO10"] | iglas#7 |
| 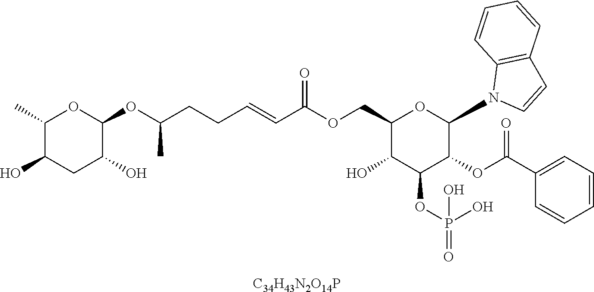 [SMILES: C[C@H](CC/C=C/C(=O)OC[C@H]1O[C@@H](n2ccc3ccccc32)[C@H](OC(=O)c2ccccc2)[C@@H](OP(=O)(O)O)[C@@H]1O)O[C@@H]1O[C@@H](C)[C@H](O)C[C@H]1O ; "C34H43N2O14P"] | iglas#701 |
| 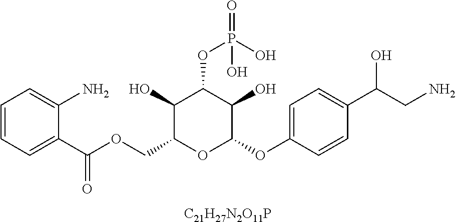 [SMILES: NCC(O)c1ccc(O[C@@H]2O[C@H](COC(=O)c3ccccc3N)[C@@H](O)[C@H](OP(=O)(O)O)[C@H]2O)cc1 ; "C21H27N2O11P"] | oglu#401 |
| 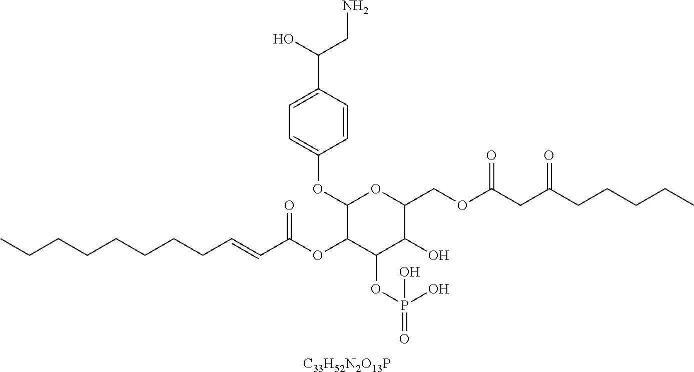 [SMILES: CCCCCCCC/C=C/C(=O)OC1C(Oc2ccc(C(O)CN)cc2)OC(COC(=O)CC(=O)CCCCC)C(O)C1OP(=O)(O)O ; "C33H52N2O13P"] | oglu#42 |
| 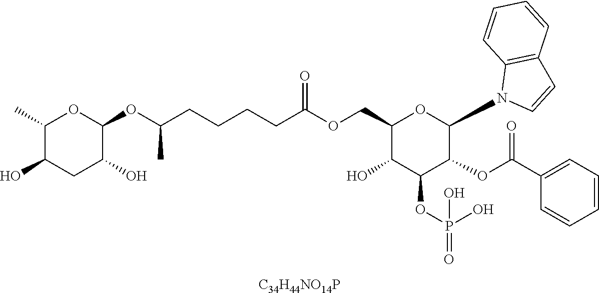 [SMILES: C[C@H](CCCCC(=O)OC[C@H]1O[C@@H](n2ccc3ccccc32)[C@H](OC(=O)c2ccccc2)[C@@H](OP(=O)(O)O)[C@@H]1O)O[C@@H]1O[C@@H](C)[C@H](O)C[C@H]1O ; "C34H44NO14P"] | iglas#702 |
| 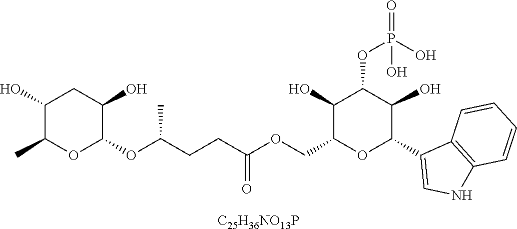 | iglas#91 |
| 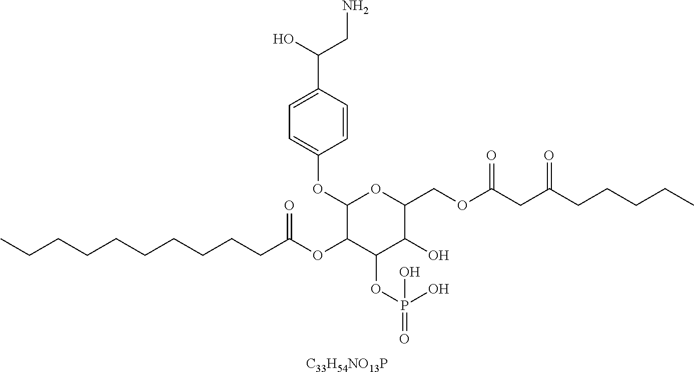 [SMILES: CCCCCCCCCCC(=O)OC1C(Oc2ccc(C(O)CN)cc2)OC(COC(=O)CC(=O)CCCCC)C(O)C1OP(=O)(O)O ; "C33H54NO13P"] | oglu#44 |
| 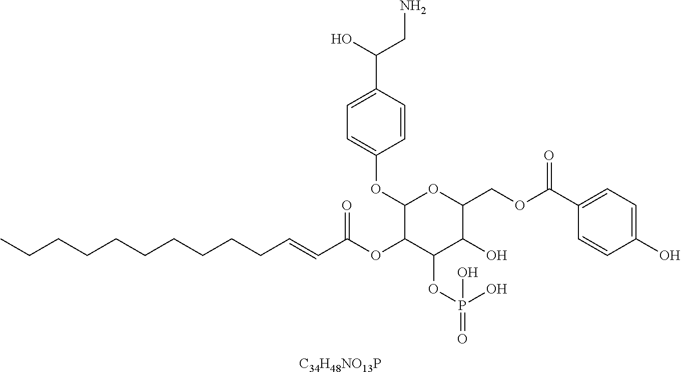 [SMILES: CCCCCCCCCC/C=C/C(=O)OC1C(Oc2ccc(C(O)CN)cc2)OC(COC(=O)c2ccc(O)cc2)C(O)C1OP(=O)(O)O ; "C34H48NO13P"] | oglu#46 |
| 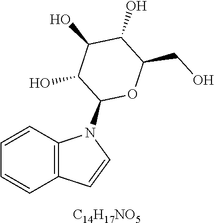 [SMILES: OC[C@H]1O[C@@H](n2ccc3ccccc32)[C@H](O)[C@@H](O)[C@@H]1O ; "C14H17NO5"] | iglu#1 |
| 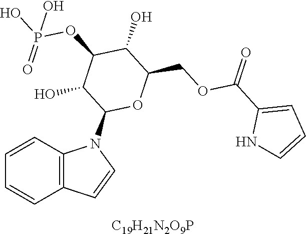 [SMILES: O=C(OC[C@H]1O[C@@H](n2ccc3ccccc32)[C@H](O)[C@@H](OP(=O)(O)O)[C@@H]1O)c1ccc[nH]1 ; "C19H21N2O9P"] | iglu#10 |
| 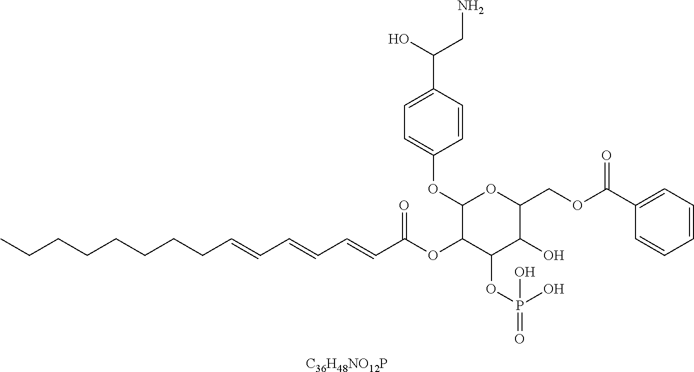 [SMILES: CCCCCCCC/C=C/C=C/C=C/C(=O)OC1C(Oc2ccc(C(O)CN)cc2)OC(COC(=O)c2ccccc2)C(O)C1OP(=O)(O)O ; "C36H48NO12P"] | oglu#48 |
| 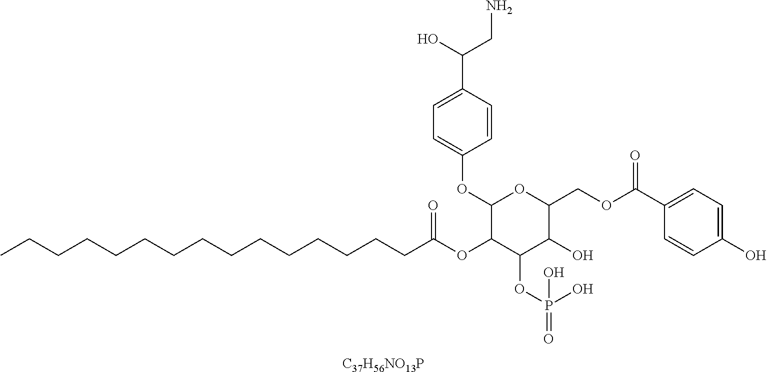 [SMILES: CCCCCCCCCCCCCCCC(=O)OC1C(Oc2ccc(C(O)CN)cc2)OC(COC(=O)c2ccc(O)cc2)C(O)C1OP(=O)(O)O ; "C37H56NO13P"] | oglu#50 |
| 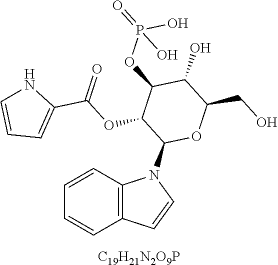 [SMILES: O=C(O[C@@H]1[C@@H](OP(=O)(O)O)[C@H](O)[C@@H](CO)O[C@H]1n1ccc2ccccc21)c1ccc[nH]1 ; "C19H21N2O9P"] | iglu#101 |
| 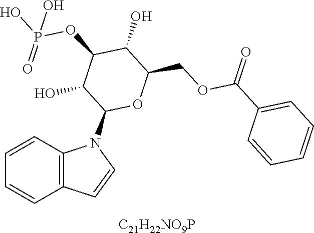 | iglu#12 |
| 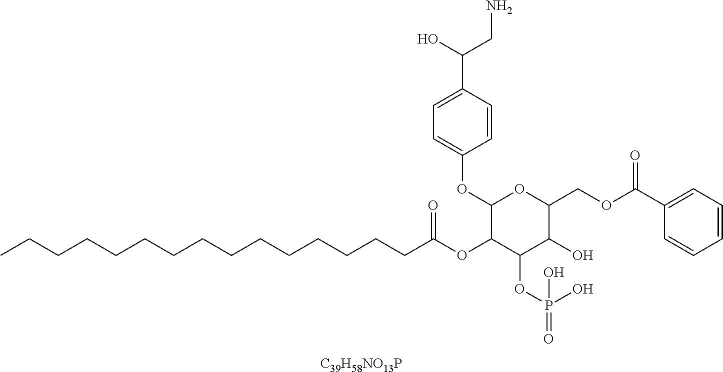 [SMILES: CCCCCCCCCCCCCCCC(=O)OC1C(Oc2ccc(C(O)CN)cc2)OC(COC(=O)c2ccccc2)C(O)C1OP(=O)(O)O ; "C39H58NO13P"] | oglu#52 |
| 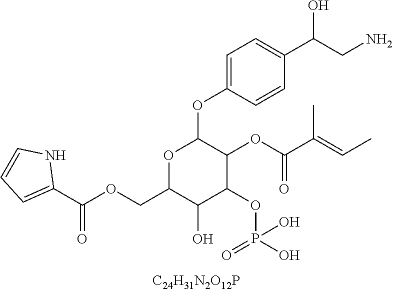 [SMILES: C/C=C(\C)C(=O)OC1C(Oc2ccc(C(O)CN)cc2)OC(COC(=O)c2ccc[nH]2)C(O)C1OP(=O)(O)O ; "C24H31N2O12P"] | oglu#601 |
| 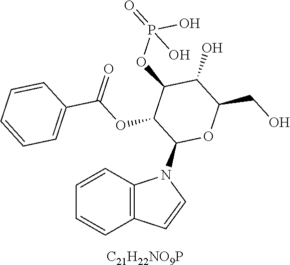 | iglu#121 |
| 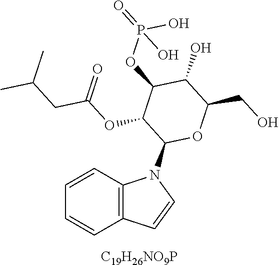 | iglu#141 |
| 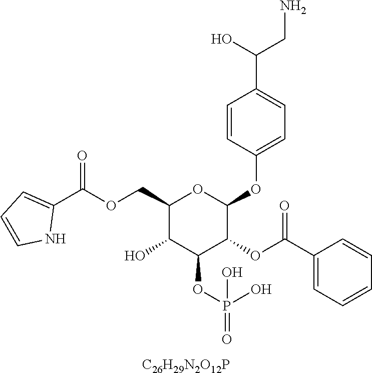 [SMILES: NCC(O)c1ccc(O[C@@H]2O[C@H](COC(=O)c3ccc[nH]3)[C@@H](O)[C@H](OP(=O)(O)O)[C@H]2OC(=O)c2ccccc2)cc1 ; "C26H29N2O12P"] | oglu#7 |
| 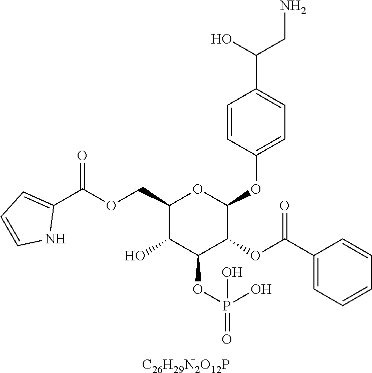 [SMILES: NCC(O)c1ccc(O[C@@H]2O[C@H](COC(=O)c3ccc[nH]3)[C@@H](O)[C@H](OP(=O)(O)O)[C@H]2OC(=O)c2ccccc2)cc1 ; "C26H29N2O12P"] | oglu#8 |
| 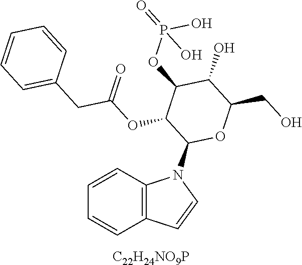 | iglu#16 |
| 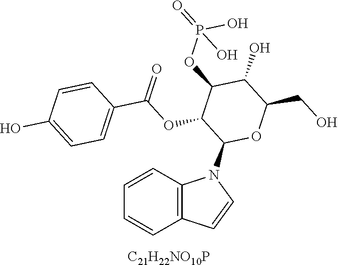 | iglu#181 |
| 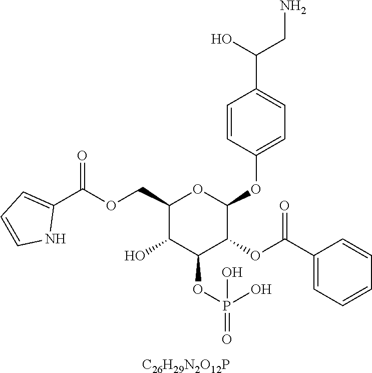 [SMILES: NCC(O)c1ccc(O[C@@H]2O[C@H](COC(=O)c3ccc[nH]3)[C@@H](O)[C@H](OP(=O)(O)O)[C@H]2OC(=O)c2ccccc2)cc1 ; "C26H29N2O12P"] | oglu#9 |
| 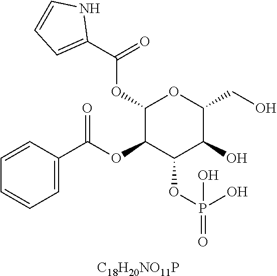 [SMILES: O=C(O[C@H]1[C@H](OC(=O)c2ccc[nH]2)O[C@H](CO)[C@@H](O)[C@@H]1OP(=O)(O)O)c1ccccc1 ; "C18H20NO11P"] | pyglu#201 |
| 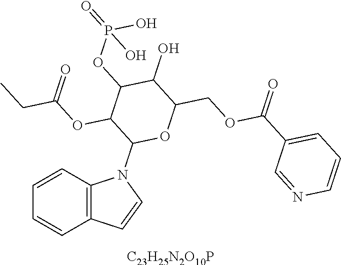 [SMILES: CCC(=O)OC1C(OP(=O)(O)O)C(O)C(COC(=O)c2cccnc2)OC1n1ccc2ccccc21 ; "C23H25N2O10P"] | iglu#19 |
| 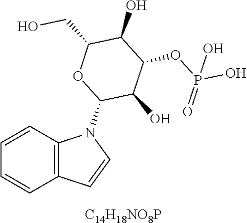 | iglu#2 |
| 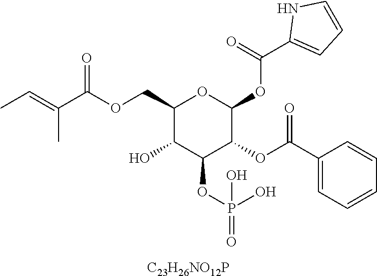 [SMILES: C/C=C(\C)C(=O)OC[C@H]1O[C@@H](OC(=O)c2ccc[nH]2)[C@H](OC(=O)c2ccccc2)[C@@H](OP(=O)(O)O)[C@@H]1O ; "C23H26NO12P"] | pyglu#4 |
| 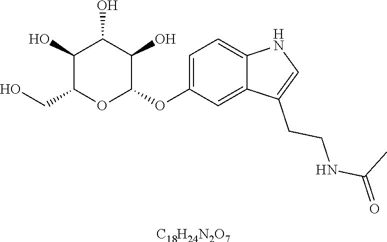 [SMILES: CC(=O)NCCc1c[nH]c2ccc(O[C@@H]3O[C@H](CO)[C@@H](O)[C@H](O)[C@H]3O)cc12 ; "C18H24N2O7"] | sngl#1 |
| 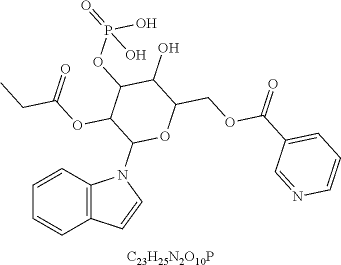 [SMILES: CCC(=O)OC1C(OP(=O)(O)O)C(O)C(COC(=O)c2cccnc2)OC1n1ccc2ccccc21 ; "C23H25N2O10P"] | iglu#21 |
| 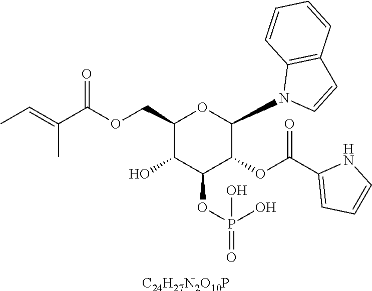 [SMILES: C/C=C(\C)C(=O)OC[C@H]1O[C@@H](n2ccc3ccccc32)[C@H](OC(=O)c2ccc[nH]2)[C@@H](OP(=O)(O)O)[C@@H]1O ; "C24H27N2O10P"] | iglu#22 |
| 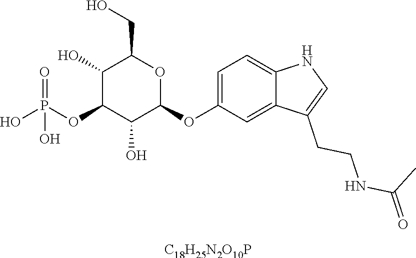 [SMILES: CC(=O)NCCc1c[nH]c2ccc(O[C@@H]3O[C@H](CO)[C@@H](O)[C@H](OP(=O)(O)O)[C@H]3O)cc12 ; "C18H25N2O10P"] | sngl#2 |
| 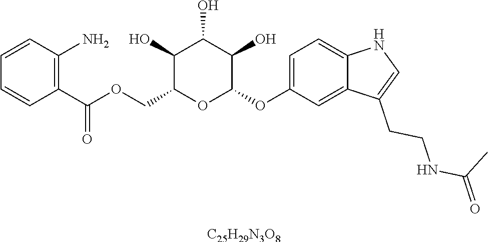 [SMILES: CC(=O)NCCc1c[nH]c2ccc(O[C@@H]3O[C@H](COC(=O)c4ccccc4N)[C@@H](O)[C@H](O)[C@H]3O)cc12 ; "C25H29N3O8"] | sngl#3 |
| 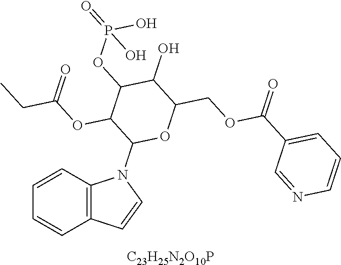 [SMILES: CCC(=O)OC1C(OP(=O)(O)O)C(O)C(COC(=O)c2cccnc2)OC1n1ccc2ccccc21 ; "C23H25N2O10P"] | iglu#23 |
| 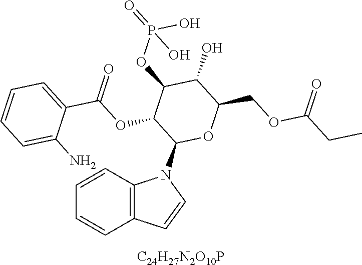 [SMILES: CCC(=O)OC[C@H]1O[C@@H](n2ccc3ccccc32)[C@H](OC(=O)c2ccccc2N)[C@@H](OP(=O)(O)O)[C@@H]1O ; "C24H27N2O10P"] | iglu#24 |
| 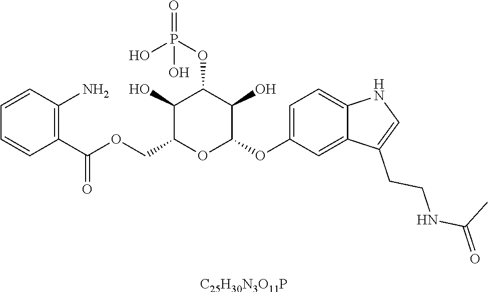 [SMILES: CC(=O)NCCc1c[nH]c2ccc(O[C@@H]3O[C@H](COC(=O)c4ccccc4N)[C@@H](O)[C@H](OP(=O)(O)O)[C@H]3O)cc12 ; "C25H30N3O11P"] | sngl#4 |
| 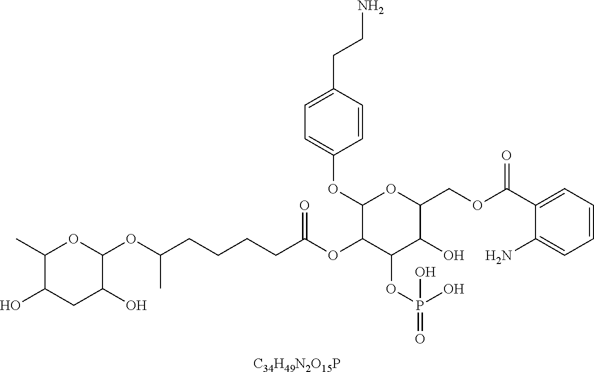 [SMILES: CC(CCCCC(=O)OC1C(Oc2ccc(CCN)cc2)OC(COC(=O)c2ccccc2N)C(O)C1OP(=O)(O)O)OC1OC(C)C(O)CC1O ; "C34H49N2O15P"] | tyglas#1 |
| 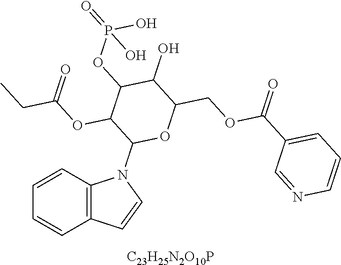 [SMILES: CCC(=O)OC1C(OP(=O)(O)O)C(O)C(COC(=O)c2cccnc2)OC1n1ccc2ccccc21 ; "C23H25N2O10P"] | iglu#25 |
| 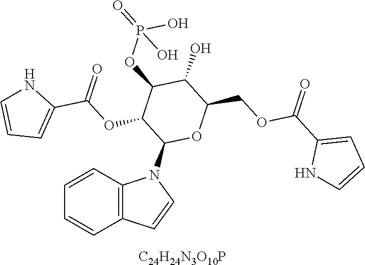 | iglu#27 |
| 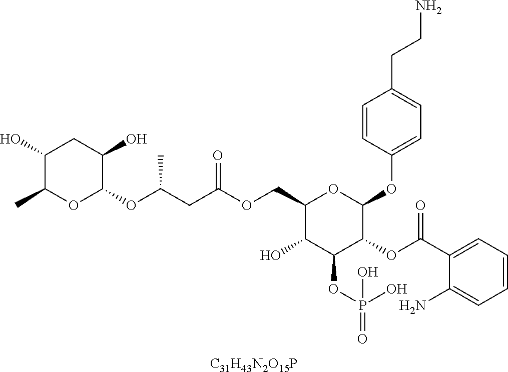 [SMILES: C[C@H](CC(=O)OC[C@H]1O[C@@H](Oc2ccc(CCN)cc2)[C@H](OC(=O)c2ccccc2N)[C@@H](OP(=O)(O)O)[C@@H]1O)O[C@@H]1O[C@@H](C)[C@H](O)C[C@H]1O ; "C31H43N2O15P"] | tyglas#11 |
| 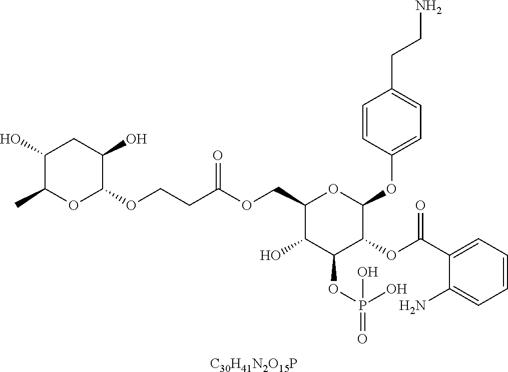 [SMILES: C[C@@H]1O[C@@H](OCCC(=O)OC[C@H]2O[C@@H](Oc3ccc(CCN)cc3)[C@H](OC(=O)c3ccccc3N)[C@@H](OP(=O)(O)O)[C@@H]2O)[C@H](O)C[C@H]1O ; "C30H41N2O15P"] | tyglas#5 |
| 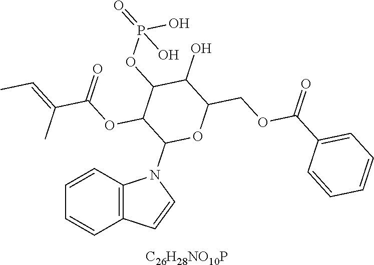 | iglu#28 |
| 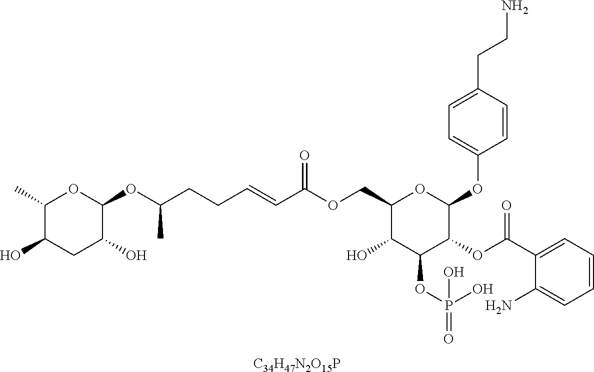 [SMILES: C[C@H](CC/C=C/C(=O)OC[C@H]1O[C@@H](Oc2ccc(CCN)cc2)[C@H](OC(=O)c2ccccc2N)[C@@H](OP(=O)(O)O)[C@@H]1O)O[C@@H]1O[C@@H](C)[C@H](O)C[C@H]1O ; "C34H47N2O15P"] | tyglas#7 |
| 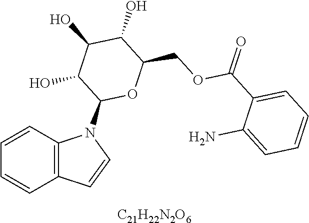 | iglu#3 |
| 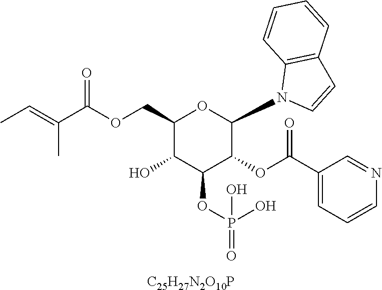 [SMILES: C/C=C(\C)C(=O)OC[C@H]1O[C@@H](n2ccc3ccccc32)[C@H](OC(=O)c2cccnc2)[C@@H](OP(=O)(O)O)[C@@H]1O ; "C25H27N2O10P"] | iglu#30 |
| 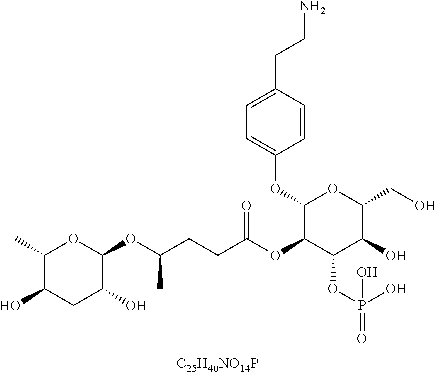 [SMILES: C[C@H](CCC(=O)O[C@H]1[C@H](Oc2ccc(CCN)cc2)O[C@H](CO)[C@@H](O)[C@@H]1OP(=O)(O)O)O[C@@H]1O[C@@H](C)[C@H](O)C[C@H]1O ; "C25H40NO14P"] | tyglas#9 |
| 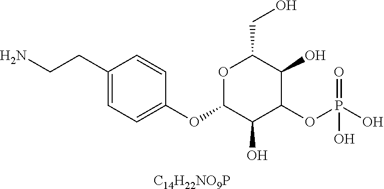 [SMILES: NCCc1ccc(O[C@@H]2O[C@H](CO)[C@@H](O)C(OP(=O)(O)O)[C@H]2O)cc1 ; "C14H22NO9P"] | tyglu#1 |
| 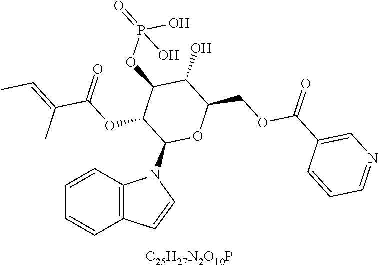 [SMILES: C/C=C(\C)C(=O)O[C@@H]1[C@@H](OP(=O)(O)O)[C@H](O)[C@@H](COC(=O)c2cccnc2)O[C@H]1n1ccc2ccccc21 ; "C25H27N2O10P"] | iglu#31 |
| 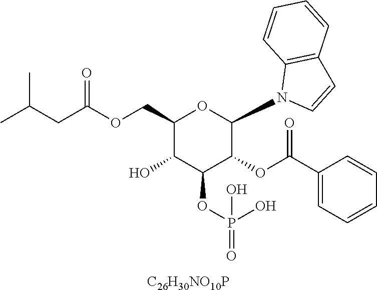 [SMILES: CC(C)CC(=O)OC[C@H]1O[C@@H](n2ccc3ccccc32)[C@H](OC(=O)c2ccccc2)[C@@H](OP(=O)(O)O)[C@@H]1O ; "C26H30NO10P"] | iglu#32 |
| 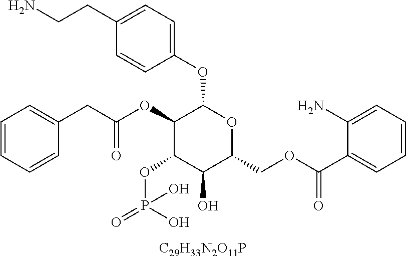 [SMILES: NCCc1ccc(O[C@@H]2O[C@H](COC(=O)c3ccccc3N)[C@@H](O)[C@H](OP(=O)(O)O)[C@H]2OC(=O)Cc2ccccc2)cc1 ; "C29H33N2O11P"] | tyglu#12 |
| 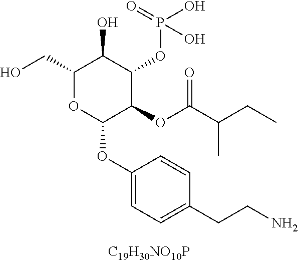 [SMILES: CCC(C)C(=O)O[C@H]1[C@H](Oc2ccc(CCN)cc2)O[C@H](CO)[C@@H](O)[C@@H]1OP(=O)(O)O ; "C19H30NO10P"] | tyglu#131 |
| 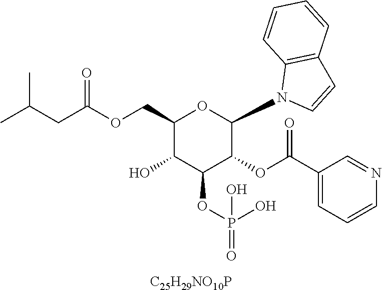 [SMILES: CC(C)CC(=O)OC[C@H]1O[C@@H](n2ccc3ccccc32)[C@H](OC(=O)c2cccnc2)[C@@H](OP(=O)(O)O)[C@@H]1O ; "C25H29NO10P"] | iglu#34 |
| 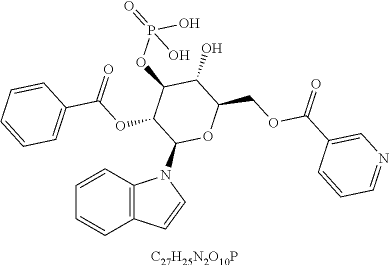 [SMILES: O=C(OC[C@H]1O[C@@H](n2ccc3ccccc32)[C@H](OC(=O)c2ccccc2)[C@@H](OP(=O)(O)O)[C@@H]1O)c1cccnc1 ; "C27H25N2O10P"] | iglu#36 |
| 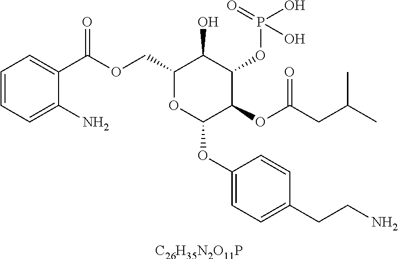 [SMILES: CC(C)CC(=O)O[C@H]1[C@H](Oc2ccc(CCN)cc2)O[C@H](COC(=O)c2ccccc2N)[C@@H](O)[C@@H]1OP(=O)(O)O ; "C26H35N2O11P"] | tyglu#14 |
| 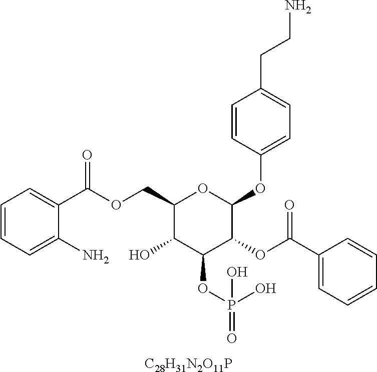 [SMILES: NCCc1ccc(O[C@@H]2O[C@H](COC(=O)c3ccccc3N)[C@@H](O)[C@H](OP(=O)(O)O)[C@H]2OC(=O)c2ccccc2)cc1 ; "C28H31N2O11P"] | tyglu#16 |
| 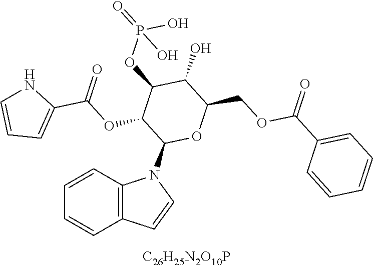 [SMILES: O=C(OC[C@H]1O[C@@H](n2ccc3ccccc32)[C@H](OC(=O)c2ccc[nH]2)[C@@H](OP(=O)(O)O)[C@@H]1O)c1ccccc1 ; "C26H25N2O10P"] | iglu#37 |
| 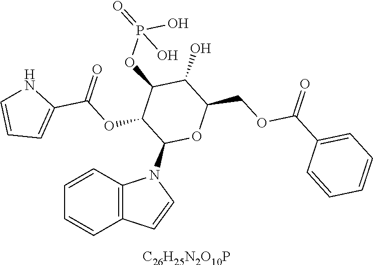 [SMILES: O=C(OC[C@H]1O[C@@H](n2ccc3ccccc32)[C@H](OC(=O)c2ccc[nH]2)[C@@H](OP(=O)(O)O)[C@@H]1O)c1ccccc1 ; "C26H25N2O10P"] | iglu#38 |
| 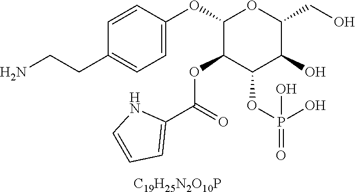 [SMILES: NCCc1ccc(O[C@@H]2O[C@H](CO)[C@@H](O)[C@H](OP(=O)(O)O)[C@H]2OC(=O)c2ccc[nH]2)cc1 ; "C19H25N2O10P"] | tyglu#181 |
| 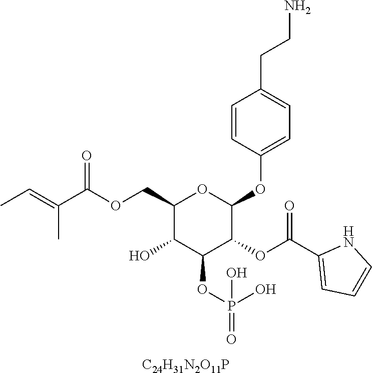 [SMILES: C/C=C(\C)C(=O)OC[C@H]1O[C@@H](Oc2ccc(CCN)cc2)[C@H](OC(=O)c2ccc[nH]2)[C@@H](OP(=O)(O)O)[C@@H]1O ; "C24H31N2O11P"] | tyglu#19 |
| 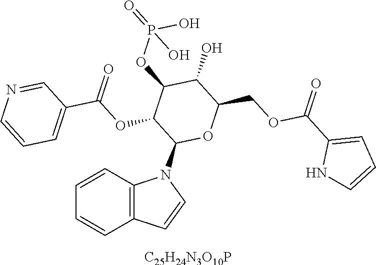 | iglu#39 |
| 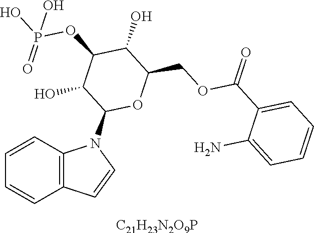 [SMILES: Nc1ccccc1C(=O)OC[C@H]1O[C@@H](n2ccc3ccccc32)[C@H](O)[C@@H](OP(=O)(O)O)[C@@H]1O ; "C21H23N2O9P"] | iglu#4 |
| 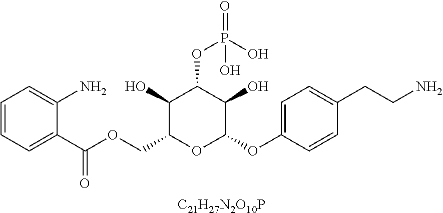 [SMILES: NCCc1ccc(O[C@@H]2O[C@H](COC(=O)c3ccccc3N)[C@@H](O)[C@H](OP(=O)(O)O)[C@H]2O)cc1 ; "C21H27N2O10P"] | tyglu#2 |
| 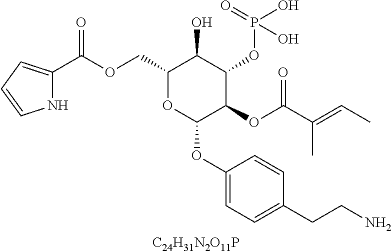 [SMILES: C/C=C(\C)C(=O)O[C@H]1[C@H](Oc2ccc(CCN)cc2)O[C@H](COC(=O)c2ccc[nH]2)[C@@H](O)[C@@H]1OP(=O)(O)O ; "C24H31N2O11P"] | tyglu#20 |
| 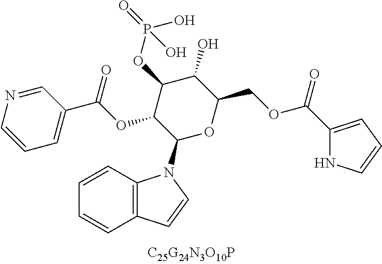 [SMILES: O=C(O[C@@H]1[C@@H](OP(=O)(O)O)[C@H](O)[C@@H](COC(=O)c2ccc[nH]2)O[C@H]1n1ccc2ccccc21)c1cccnc1] | iglu#40 |
| 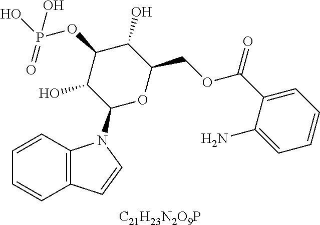 [SMILES: Nc1ccccc1C(=O)OC[C@H]1O[C@@H](n2ccc3ccccc32)[C@H](O)[C@@H](OP(=O)(O)O)[C@@H]1O ; "C21H23N2O9P"] | iglu#401 |
| 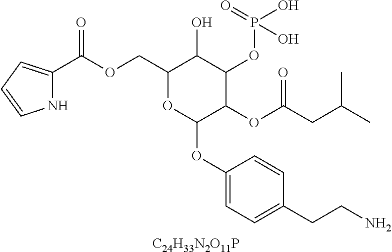 [SMILES: CC(C)CC(=O)OC1C(Oc2ccc(CCN)cc2)OC(COC(=O)c2ccc[nH]2)C(O)C1OP(=O)(O)O ; "C24H33N2O11P"] | tyglu#22 |
| 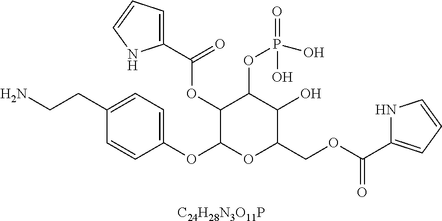 [SMILES: NCCc1ccc(OC2OC(COC(=O)c3ccc[nH]3)C(O)C(OP(=O)(O)O)C2OC(=O)c2ccc[nH]2)cc1 ; "C24H28N3O11P"] | tyglu#24 |
| 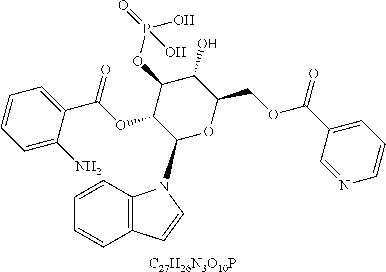 | iglu#41 |
| 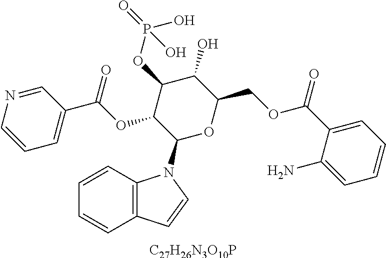 | iglu#41 |
| 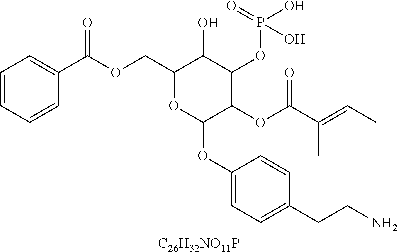 [SMILES: C/C=C(\C)C(=O)OC1C(Oc2ccc(CCN)cc2)OC(COC(=O)c2ccccc2)C(O)C1OP(=O)(O)O ; "C26H32NO11P"] | tyglu#26 |
| 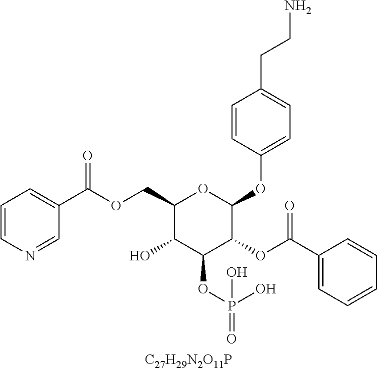 [SMILES: NCCc1ccc(O[C@@H]2O[C@H](COC(=O)c3cccnc3)[C@@H](O)[C@H](OP(=O)(O)O)[C@H]2OC(=O)c2ccccc2)cc1 ; "C27H29N2O11P"] | tyglu#28 |
| 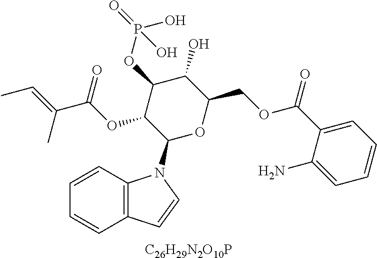 [SMILES: C/C=C(\C)C(=O)O[C@@H]1[C@@H](OP(=O)(O)O)[C@H](O)[C@@H](COC(=O)c2ccccc2N)O[C@H]1n1ccc2ccccc21 ; "C26H29N2O10P"] | iglu#42 |
| 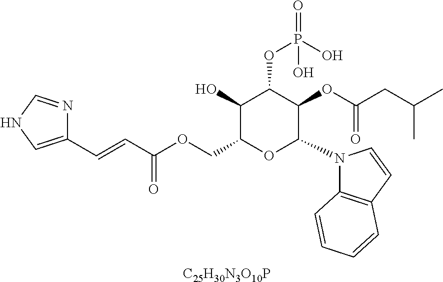 [SMILES: CC(C)CC(=O)O[C@@H]1[C@@H](OP(=O)(O)O)[C@H](O)[C@@H](COC(=O)/C=C/c2c[nH]cn2)O[C@H]1n1ccc2ccccc21 ; "C25H30N3O10P"] | iglu#44 |
| 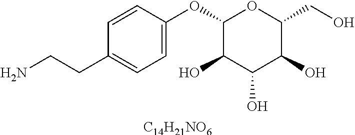 [SMILES: NCCc1ccc(O[C@@H]2O[C@H](CO)[C@@H](O)[C@H](O)[C@H]2O)cc1 ; "C14H21NO6"] | tyglu#3 |
| 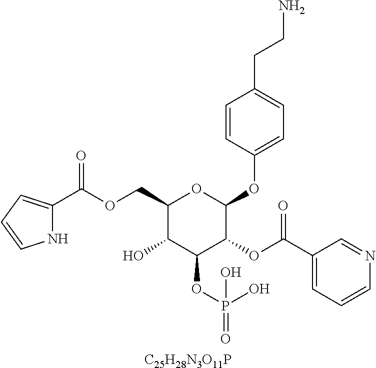 [SMILES: NCCc1ccc(O[C@@H]2O[C@H](COC(=O)c3ccc[nH]3)[C@@H](O)[C@H](OP(=O)(O)O)[C@H]2OC(=O)c2cccnc2)cc1 ; "C25H28N3O11P"] | tyglu#30 |
| 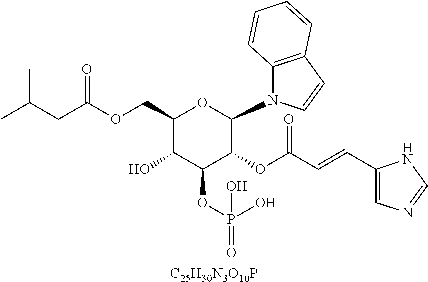 [SMILES: CC(C)CC(=O)OC[C@H]1O[C@@H](n2ccc3ccccc32)[C@H](OC(=O)/C=C/c2cnc[nH]2)[C@@H](OP(=O)(O)O)[C@@H]1O ; "C25H30N3O10P"] | iglu#45 |
| 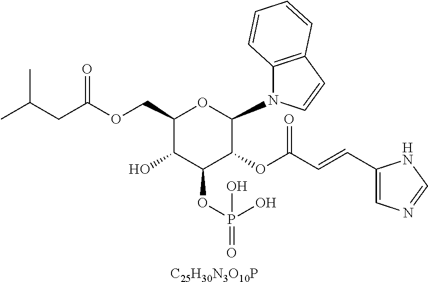 [SMILES: CC(C)CC(=O)OC[C@H]1O[C@@H](n2ccc3ccccc32)[C@H](OC(=O)/C=C/c2cnc[nH]2)[C@@H](OP(=O)(O)O)[C@@H]1O ; "C25H30N3O10P"] | iglu#46 |
| 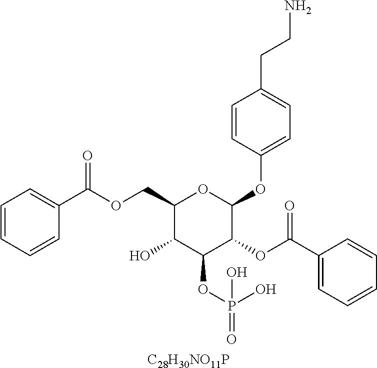 [SMILES: NCCc1ccc(O[C@@H]2O[C@H](COC(=O)c3ccccc3)[C@@H](O)[C@H](OP(=O)(O)O)[C@H]2OC(=O)c2ccccc2)cc1 ; "C28H30NO11P"] | tyglu#32 |
| 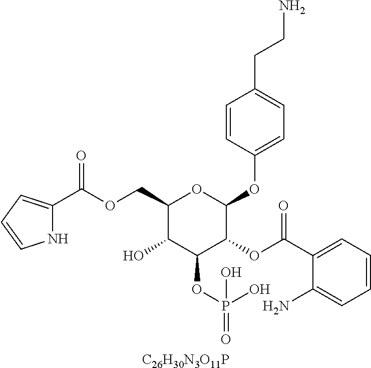 [SMILES: NCCc1ccc(O[C@@H]2O[C@H](COC(=O)c3ccc[nH]3)[C@@H](O)[C@H](OP(=O)(O)O)[C@H]2OC(=O)c2ccccc2N)cc1 ; "C26H30N3O11P"] | tyglu#34 |
| 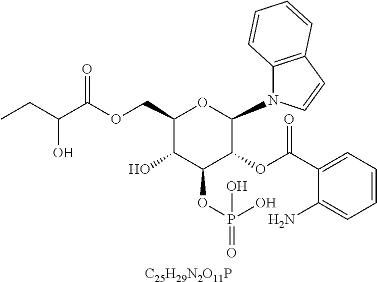 [SMILES: CCC(O)C(=O)OC[C@H]1O[C@@H](n2ccc3ccccc32)[C@H](OC(=O)c2ccccc2N)[C@@H](OP(=O)(O)O)[C@@H]1O ; "C25H29N2O11P"] | iglu#47 |
| 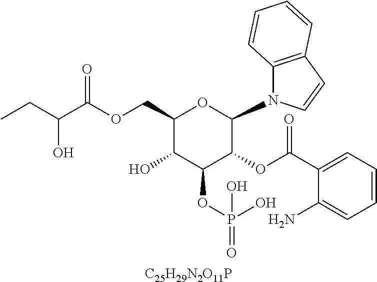 [SMILES: CCC(O)C(=O)OC[C@H]1O[C@@H](n2ccc3ccccc32)[C@H](OC(=O)c2ccccc2N)[C@@H](OP(=O)(O)O)[C@@H]1O ; "C25H29N2O11P"] | iglu#48 |
| 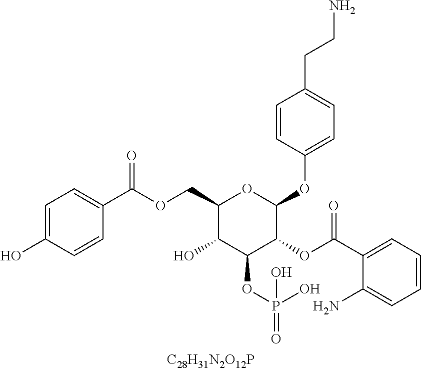 [SMILES: NCCc1ccc(O[C@@H]2O[C@H](COC(=O)c3ccc(O)cc3)[C@@H](O)[C@H](OP(=O)(O)O)[C@H]2OC(=O)c2ccccc2N)cc1 ; "C28H31N2O12P"] | tyglu#35 |
| 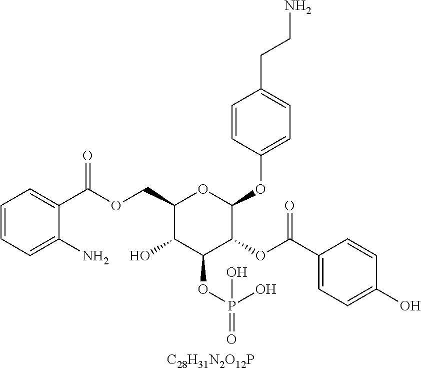 [SMILES: NCCc1ccc(O[C@@H]2O[C@H](COC(=O)c3ccccc3N)[C@@H](O)[C@H](OP(=O)(O)O)[C@H]2OC(=O)c2ccc(O)cc2)cc1 ; "C28H31N2O12P"] | tyglu#36 |
| 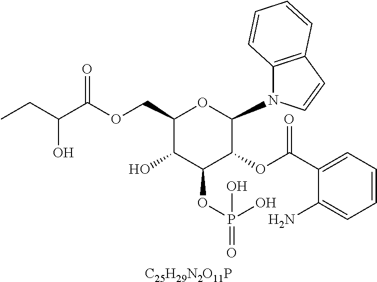 [SMILES: CCC(O)C(=O)OC[C@H]1O[C@@H](n2ccc3ccccc32)[C@H](OC(=O)c2ccccc2N)[C@@H](OP(=O)(O)O)[C@@H]1O ; "C25H29N2O11P"] | iglu#49 |
| 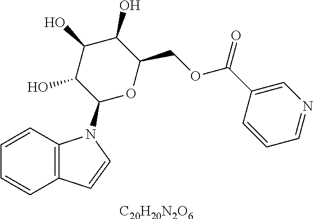 | iglu#5 |
| 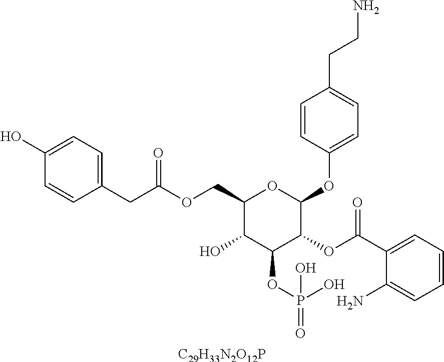 [SMILES: NCCc1ccc(O[C@@H]2O[C@H](COC(=O)Cc3ccc(O)cc3)[C@@H](O)[C@H](OP(=O)(O)O)[C@H]2OC(=O)c2ccccc2N)cc1 ; "C29H33N2O12P"] | tyglu#37 |
| 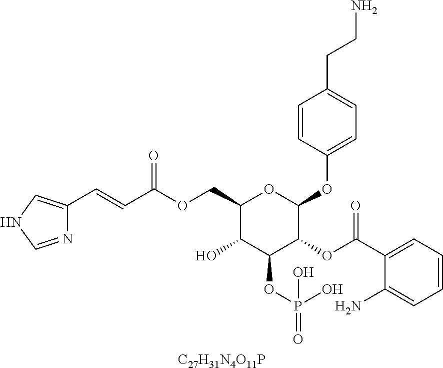 [SMILES: NCCc1ccc(O[C@@H]2O[C@H](COC(=O)/C=C/c3c[nH]cn3)[C@@H](O)[C@H](OP(=O)(O)O)[C@H]2OC(=O)c2ccccc2N)cc1 ; "C27H31N4O11P"] | tyglu#38 |
| 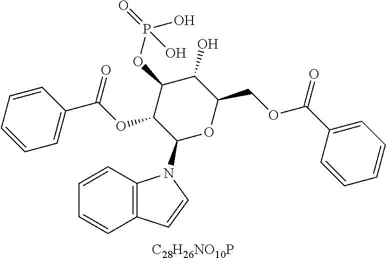 | iglu#50 |
| 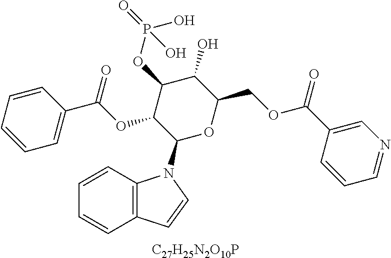 [SMILES: O=C(OC[C@H]1O[C@@H](n2ccc3ccccc32)[C@H](OC(=O)c2ccccc2)[C@@H](OP(=O)(O)O)[C@@H]1O)c1cccnc1 ; "C27H25N2O10P"] | iglu#52 |
| 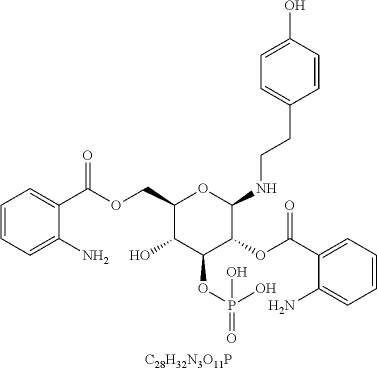 [SMILES: Nc1ccccc1C(=O)OC[C@H]1O[C@@H](NCCc2ccc(O)cc2)[C@H](OC(=O)c2ccccc2N)[C@@H](OP(=O)(O)O)[C@@H]1O ; "C28H32N3O11P"] | tyglu#4 |
| 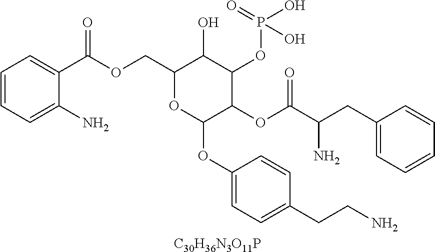 [SMILES: NCCc1ccc(OC2OC(COC(=O)c3ccccc3N)C(O)C(OP(=O)(O)O)C2OC(=O)C(N)Cc2ccccc2)cc1 ; "C30H36N3O11P"] | tyglu#42 |
| 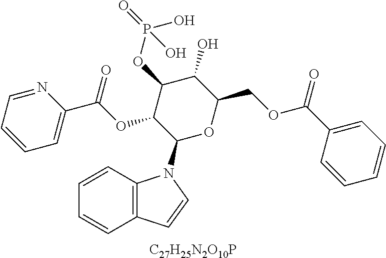 [SMILES: O=C(OC[C@H]1O[C@@H](n2ccc3ccccc32)[C@H](OC(=O)c2ccccn2)[C@@H](OP(=O)(O)O)[C@@H]1O)c1ccccc1 ; "C27H25N2O10P"] | iglu#53 |
| 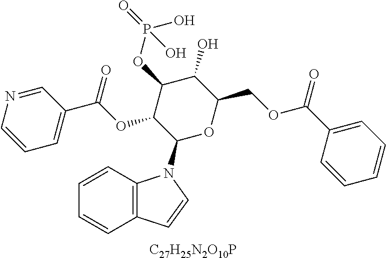 [SMILES: O=C(OC[C@H]1O[C@@H](n2ccc3ccccc32)[C@H](OC(=O)c2cccnc2)[C@@H](OP(=O)(O)O)[C@@H]1O)c1ccccc1 ; "C27H25N2O10P"] | iglu#54 |
| 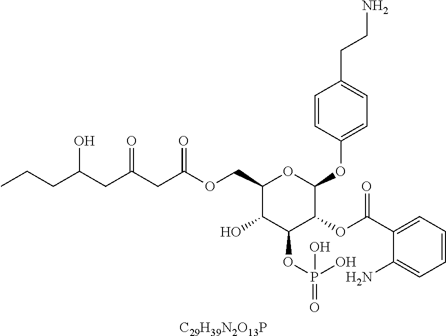 [SMILES: CCCC(O)CC(=O)CC(=O)OC[C@H]1O[C@@H](Oc2ccc(CCN)cc2)[C@H](OC(=O)c2ccccc2N)[C@@H](OP(=O)(O)O)[C@@H]1O ; "C29H39N2O13P"] | tyglu#45 |
| 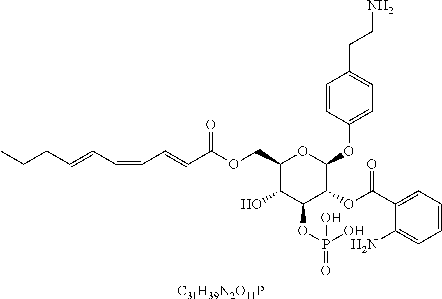 [SMILES: CCC/C=C/C=C\C=C\C(=O)OC[C@H]1O[C@@H](Oc2ccc(CCN)cc2)[C@H](OC(=O)c2ccccc2N)[C@@H](OP(=O)(O)O)[C@@H]1O ; "C31H39N2O11P"] | tyglu#50 |
| 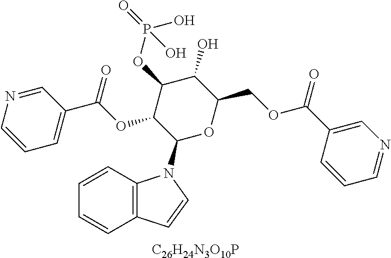 | iglu#56 |
| 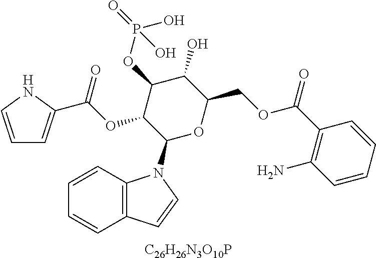 | iglu#57 |
| 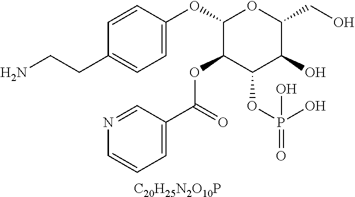 [SMILES: NCCc1ccc(O[C@@H]2O[C@H](CO)[C@@H](O)[C@H](OP(=O)(O)O)[C@H]2OC(=O)c2cccnc2)cc1 ; "C20H25N2O10P"] | tyglu#501 |
| 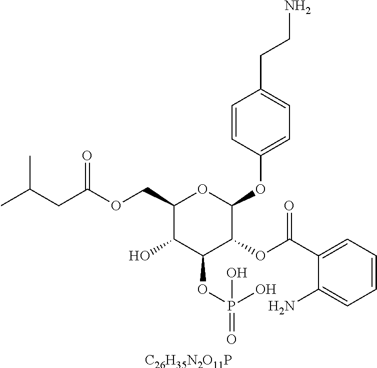 [SMILES: CC(C)CC(=O)OC[C@H]1O[C@@H](Oc2ccc(CCN)cc2)[C@H](OC(=O)c2ccccc2N)[C@@H](OP(=O)(O)O)[C@@H]1O ; "C26H35N2O11P"] | tyglu#52 |
| 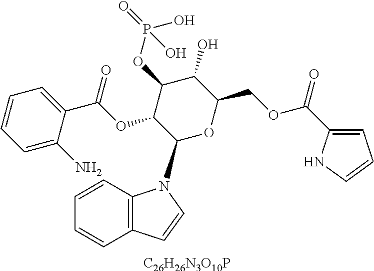 | iglu#58 |
| 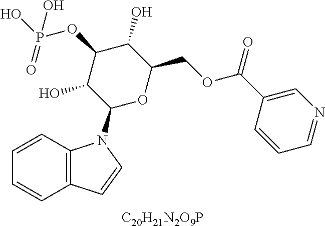 [SMILES: O=C(OC[C@H]1O[C@@H](n2ccc3ccccc32)[C@H](O)[C@@H](OP(=O)(O)O)[C@@H]1O)c1cccnc1 ; "C20H21N2O9P"] | iglu#6 |
| 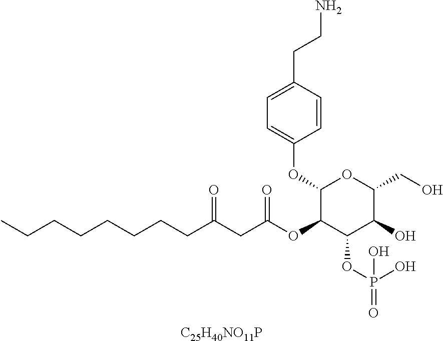 [SMILES: CCCCCCCCC(=O)CC(=O)O[C@H]1[C@H](Oc2ccc(CCN)cc2)O[C@H](CO)[C@@H](O)[C@@H]1OP(=O)(O)O ; "C25H40NO11P"] | tyglu#53 |
| 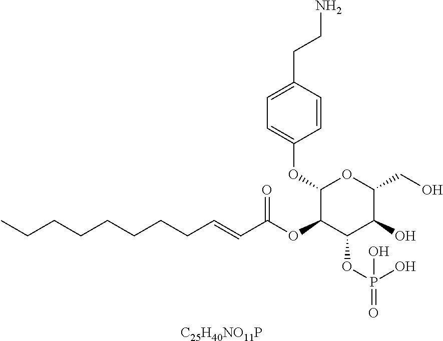 [SMILES: CCCCCCCC/C=C/C(=O)O[C@H]1[C@H](Oc2ccc(CCN)cc2)O[C@H](CO)[C@@H](O)[C@@H]1OP(=O)(O)O ; "C25H40NO11P"] | tyglu#54 |
| 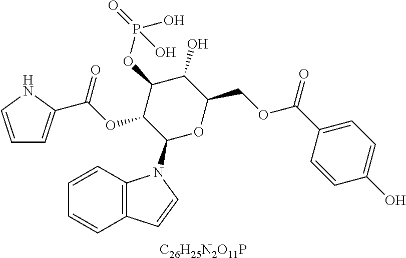 [SMILES: O=C(OC[C@H]1O[C@@H](n2ccc3ccccc32)[C@H](OC(=O)c2ccc[nH]2)[C@@H](OP(=O)(O)O)[C@@H]1O)c1ccc(O)cc1 ; "C26H25N2O11P"] | iglu#60 |
| 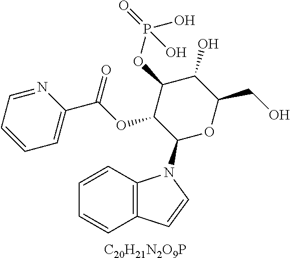 [SMILES: O=C(O[C@@H]1[C@@H](OP(=O)(O)O)[C@H](O)[C@@H](CO)O[C@H]1n1ccc2ccccc21)c1ccccn1 ; "C20H21N2O9P"] | iglu#601 |
| 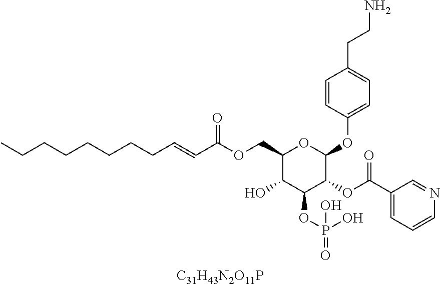 [SMILES: CCCCCCCC/C=C/C(=O)OC[C@H]1O[C@@H](Oc2ccc(CCN)cc2)[C@H](OC(=O)c2cccnc2)[C@@H](OP(=O)(O)O)[C@@H]1O ; "C31H43N2O11P"] | tyglu#56 |
| 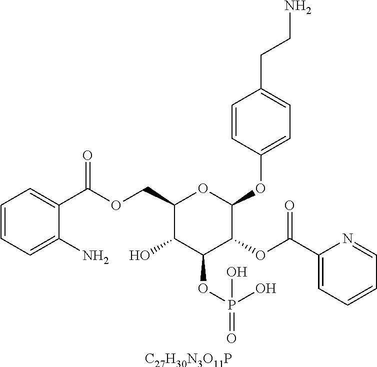 [SMILES: NCCc1ccc(O[C@@H]2O[C@H](COC(=O)c3ccccc3N)[C@@H](O)[C@H](OP(=O)(O)O)[C@H]2OC(=O)c2ccccn2)cc1 ; "C27H30N3O11P"] | tyglu#6 |
| 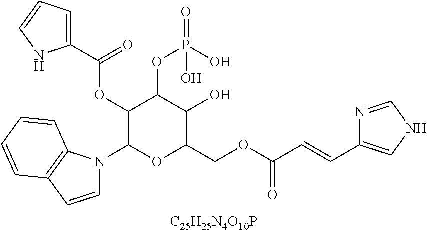 [SMILES: O=C(/C=C/c1c[nH]cn1)OCC1OC(n2ccc3ccccc32)C(OC(=O)c2ccc[nH]2)C(OP(=O)(O)O)C1O ; "C25H25N4O10P"] | iglu#62 |
| 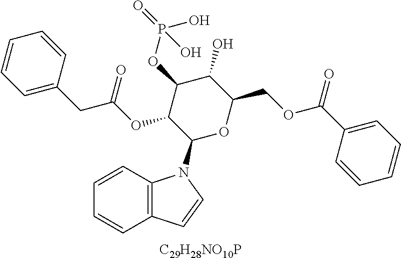 | iglu#64 |
| 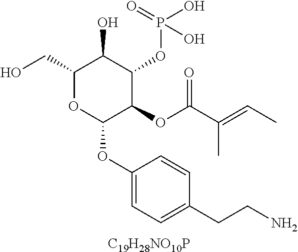 | tyglu#701 |
| 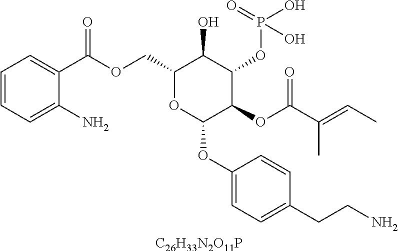 [SMILES: C/C=C(\C)C(=O)O[C@H]1[C@H](Oc2ccc(CCN)cc2)O[C@H](COC(=O)c2ccccc2N)[C@@H](O)[C@@H]1OP(=O)(O)O ; "C26H33N2O11P"] | tyglu#8 |
| 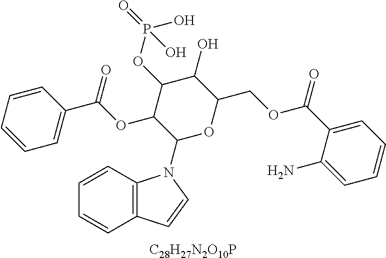 [SMILES: Nc1ccccc1C(=O)OCC1OC(n2ccc3ccccc32)C(OC(=O)c2ccccc2)C(OP(=O)(O)O)C1O ; "C28H27N2O10P"] | iglu#65 |
| 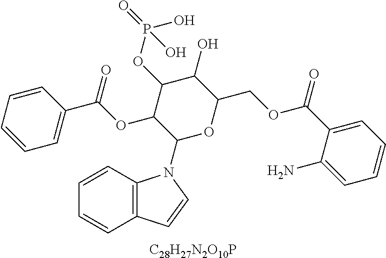 [SMILES: Nc1ccccc1C(=O)OCC1OC(n2ccc3ccccc32)C(OC(=O)c2ccccc2)C(OP(=O)(O)O)C1O ; "C28H27N2O10P"] | iglu#66 |
| 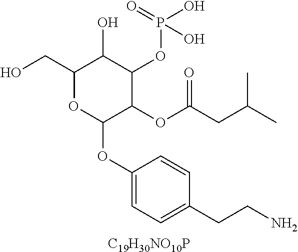 | tyglu#9 |
| 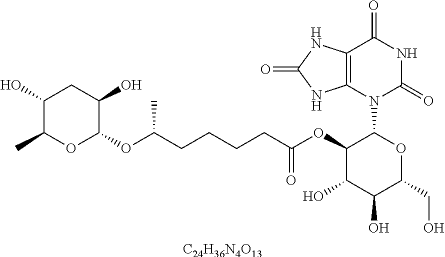 [SMILES: C[C@H](CCCCC(=O)O[C@@H]1[C@@H](O)[C@H](O)[C@@H](CO)O[C@H]1n1c(=O)[nH]c(=O)c2[nH]c(=O)[nH]c21)O[C@@H]1O[C@@H](C)[C@H](O)C[C@H]1O ; "C24H36N4O13"] | uglas#1 |
| 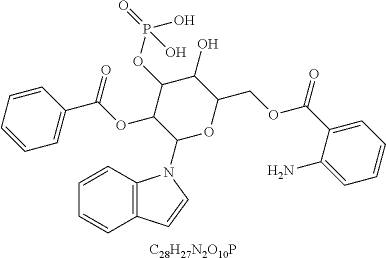 [SMILES: Nc1ccccc1C(=O)OCC1OC(n2ccc3ccccc32)C(OC(=O)c2ccccc2)C(OP(=O)(O)O)C1O ; "C28H27N2O10P"] | iglu#68 |
| 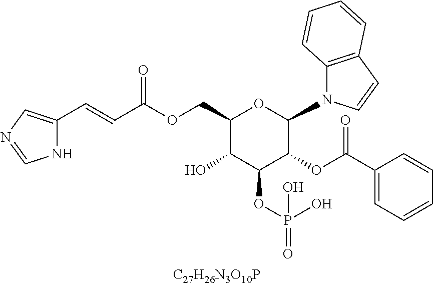 [SMILES: O=C(/C=C/c1cnc[nH]1)OC[C@H]1O[C@@H](n2ccc3ccccc32)[C@H](OC(=O)c2ccccc2)[C@@H](OP(=O)(O)O)[C@@H]1O ; "C27H26N3O10P"] | iglu#69 |
| 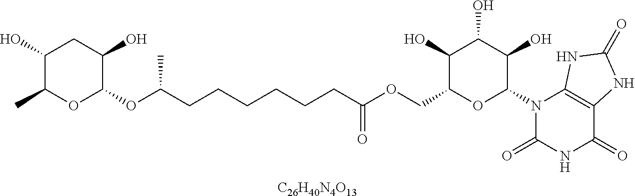 [SMILES: C[C@H](CCCCCCC(=O)OC[C@H]1O[C@@H](n2c(=O)[nH]c(=O)c3[nH]c(=O)[nH]c32)[C@H](O)[C@@H](O)[C@@H]1O)O[C@@H]1O[C@@H](C)[C@H](O)C[C@H]1O ; "C26H40N4O13"] | uglas#104 |
| 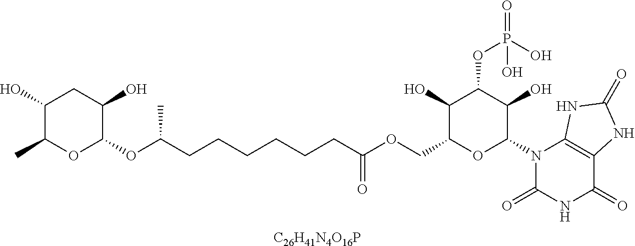 [SMILES: C[C@H](CCCCCCC(=O)OC[C@H]1O[C@@H](n2c(=O)[nH]c(=O)c3[nH]c(=O)[nH]c32)[C@H](O)[C@@H](OP(=O)(O)O)[C@@H]1O)O[C@@H]1O[C@@H](C)[C@H](O)C[C@H]1O ; "C26H41N4O16P"] | uglas#105 |
| 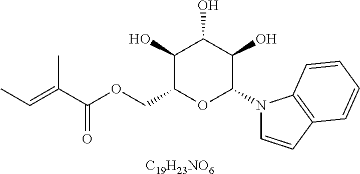 [SMILES: C/C=C(\C)C(=O)OC[C@H]1O[C@@H](n2ccc3ccccc32)[C@H](O)[C@@H](O)[C@@H]1O ; "C19H23NO6"] | iglu#7 |
| 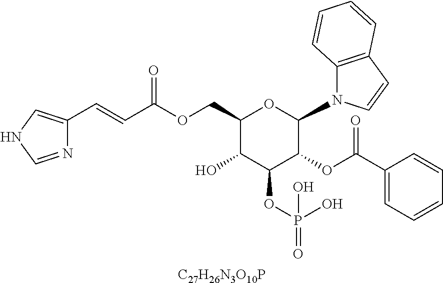 [SMILES: O=C(/C=C/c1c[nH]cn1)OC[C@H]1O[C@@H](n2ccc3ccccc32)[C@H](OC(=O)c2ccccc2)[C@@H](OP(=O)(O)O)[C@@H]1O ; "C27H26N3O10P"] | iglu#70 |
| 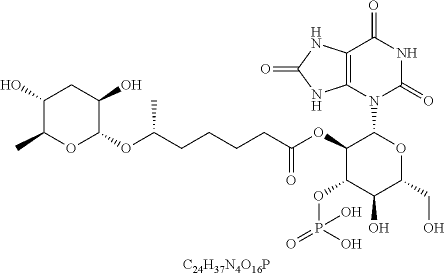 [SMILES: C[C@H](CCCCC(=O)O[C@@H]1[C@@H](OP(=O)(O)O)[C@H](O)[C@@H](CO)O[C@H]1n1c(=O)[nH]c(=O)c2[nH]c(=O)[nH]c21)O[C@@H]1O[C@@H](C)[C@H](O)C[C@H]1O ; "C24H37N4O16P"] | uglas#11 |
| 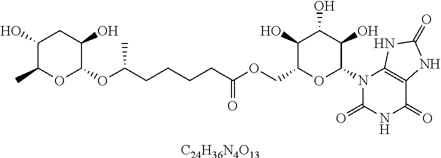 [SMILES: C[C@H](CCCCC(=O)OC[C@H]1O[C@@H](n2c(=O)[nH]c(=O)c3[nH]c(=O)[nH]c32)[C@H](O)[C@@H](O)[C@@H]1O)O[C@@H]1O[C@@H](C)[C@H](O)C[C@H]1O ; "C24H36N4O13"] | uglas#14 |
| 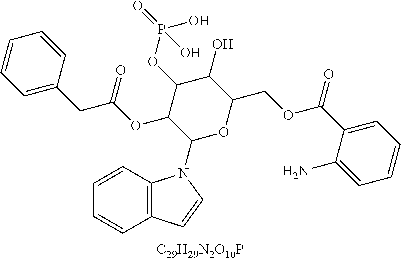 [SMILES: Nc1ccccc1C(=O)OCC1OC(n2ccc3ccccc32)C(OC(=O)Cc2ccccc2)C(OP(=O)(O)O)C1O ; "C29H29N2O10P"] | iglu#72 |
| 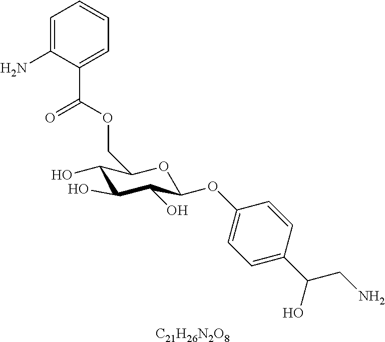 | oglu#3 |
| 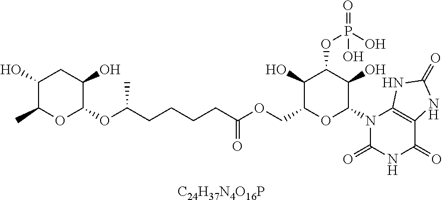 [SMILES: C[C@H](CCCCC(=O)OC[C@H]1O[C@@H](n2c(=O)[nH]c(=O)c3[nH]c(=O)[nH]c32)[C@H](O)[C@@H](OP(=O)(O)O)[C@@H]1O)O[C@@H]1O[C@@H](C)[C@H](O)C[C@H]1O ; "C24H37N4O16P"] | uglas#15 |
| 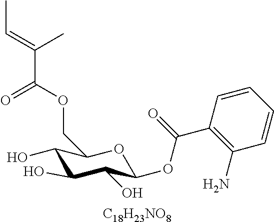 | angl#5 |
| 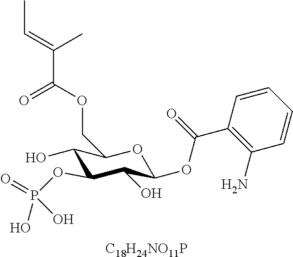 | angl#6 |
| 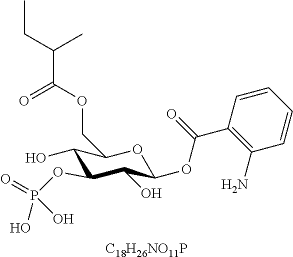 | angl#8 |
| 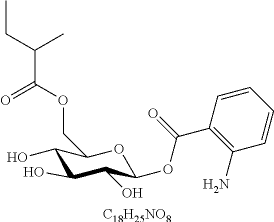 | angl#7 |
| 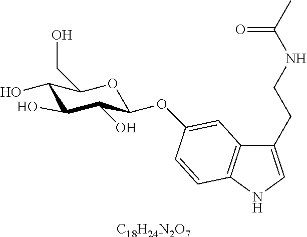 | sngl#1 |
| 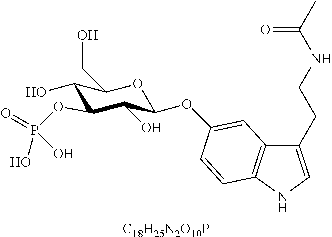 | sngl#2 |
| 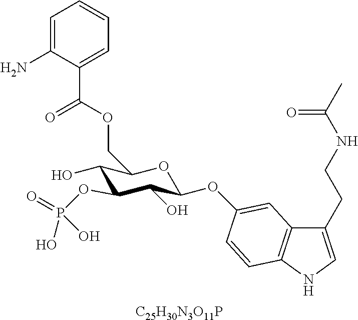 | sngl#4 |
| 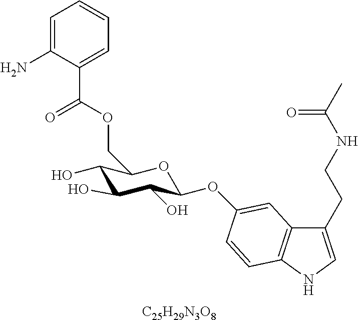 | sngl#3 |
| 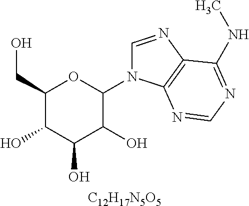 [SMILES: CNc1ncnc2c1ncn2C1O[C@H](CO)[C@@H](O)[C@H](O)C1O ; "C12H17N5O5"] | maglu#3 |
| 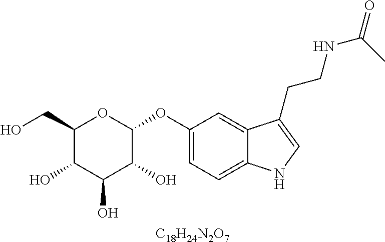 [SMILES: CC(=O)NCCc1c[nH]c2ccc(O[C@H]3O[C@H](CO)[C@@H](O)[C@H](O)[C@H]3O)cc12 ; "C18H24N2O7"] | α-sngl#1 Compound 36 |

1: A method treating a disease or disorder, comprising administering to a patient in need thereof a therapeutically effective amount of a composition comprising one or more MOGLs of Formula I: 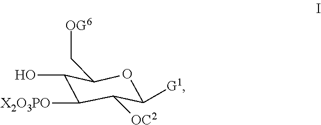
or a pharmaceutically acceptable salt thereof
wherein:
G¹ is an optionally substituted moiety selected from the group consisting of: N-linked heterocycle, —OR¹⁰ and —OC(O)R¹¹;
G² is an optionally substituted aliphatic acyl, aromatic acyl, heteroaromatic acyl, or heteroaliphatic acyl group;
X is, independently at each occurrence, selected from the group consisting of —H, an optionally substituted phosphate or polyphosphate moiety, M⁺, and Z⁺;
M⁺ is any metal cation;
Z⁺ is an organic or inorganic ‘onium’ group comprising at least one nitrogen-, phosphorous-, or sulfur-based cation;
G⁶ is an optionally substituted aliphatic acyl, aromatic acyl, heteroaromatic acyl, or heteroaliphatic acyl group; and
R¹⁰ and R¹¹ are each independently selected from the group consisting of: optionally substituted C₁₋₃₂ aliphatic, optionally substituted C₁₋₃₂ heteroaliphatic, optionally substituted aryl, and optionally substituted heteroaryl.

2: The method of claim 1, where the disease or disorder is a neurological disease, and wherein the composition comprises one or more MOGLs selected from the group consisting of: 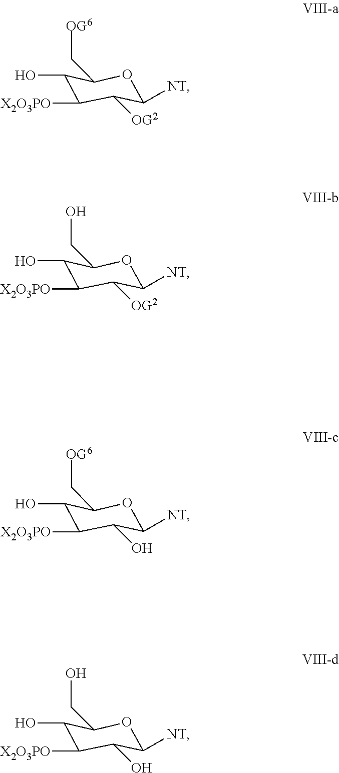
where the moiety —NT comprises a neurotransmitter, or a derivative or precursor of a neurotransmitter linked to the glucose through any suitable atom.

3: The method of claim 2, wherein the —NT comprises a monoamine neurotransmitter or a derivative or precursor thereof.

4: The method of claim 2, wherein the —NT is selected from the group consisting of: catecholamine neurotransmitters or derivatives or precursors thereof, dopamine, norepinepherine, epinepherine, histamine, serotonin, tryptamine, phenethylamine, N-methylphenethylamine, phenethanolamine, m-tyramine, p-tyramine, 3-methoxytyramine, N-methyltyramine, 3-indothyronamine, m-octopamine, p-octopamine, and synepherine.

5: The method of claim 1, where the disease or disorder is cancer, a kinase dependent disorder or disease such as hypertension, Parkinson's disease, and autoimmune disease, or a disorder that results in or arises from changes to nucleotide synthesis including, but not limited to cancer and viral diseases,
wherein the composition comprises one or more MOGLs selected from the group consisting of: 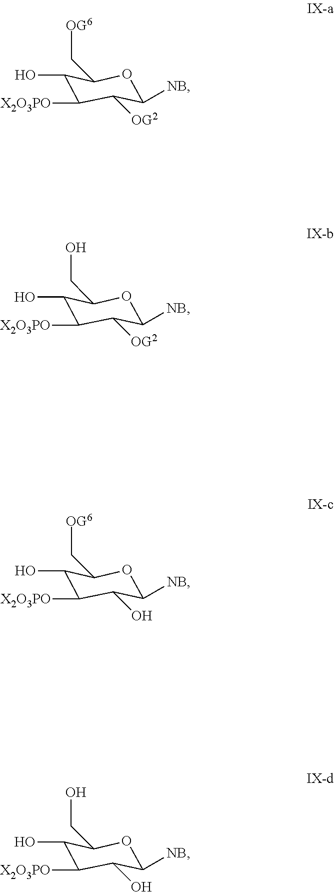
where —NB comprises an aromatic moiety, a nucleobase, or a derivative or precursor of a nucleobase linked to the glucose through any suitable atom.

6: The method of claim 5, wherein —NB comprises a nucleobase linked to the glucose through a nitrogen or oxygen atom comprising part of the nucleobase structure.

7: The method of claim 5, wherein —NB is selected from the group consisting of:

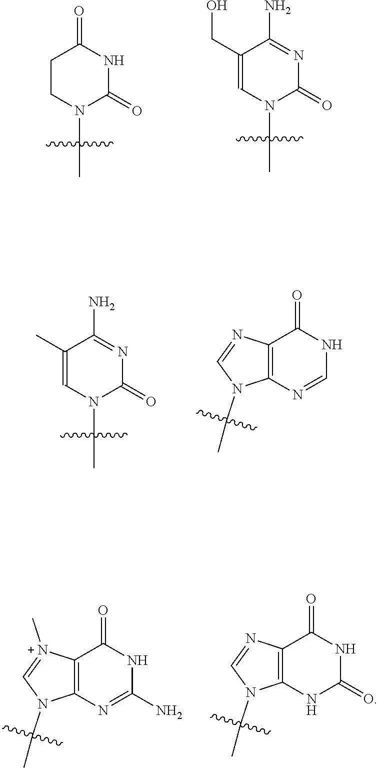

8: The method of claim 5, wherein —NB is selected from the group consisting of:

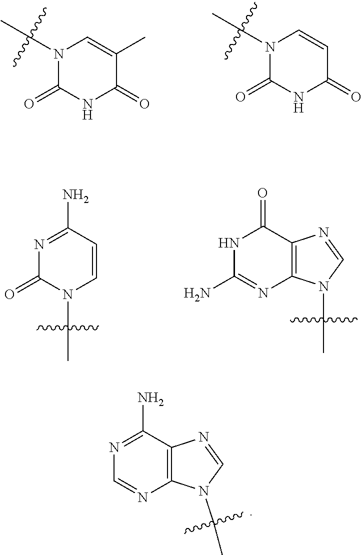

9: The method of claim 5, wherein —NB is selected from the group consisting of:

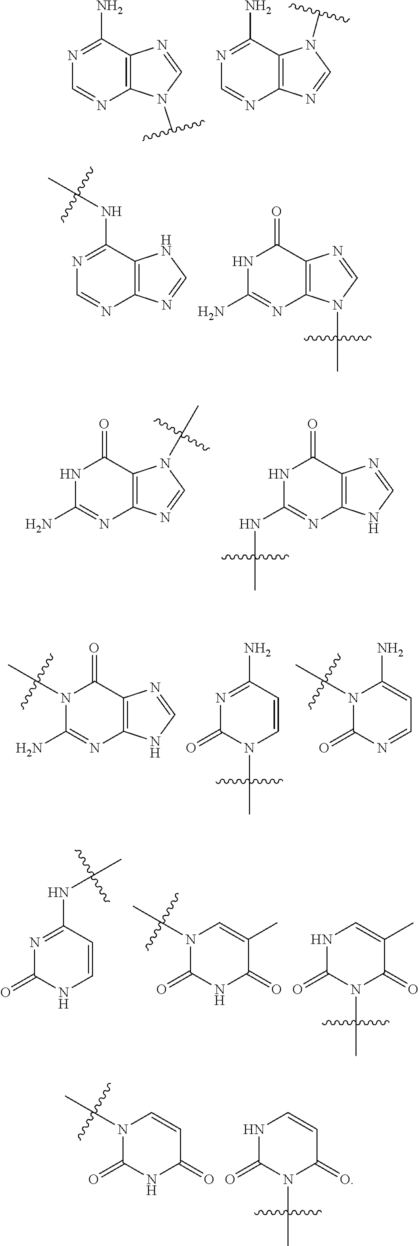

10: The method of claim 5, wherein —NB is selected from the group consisting of:

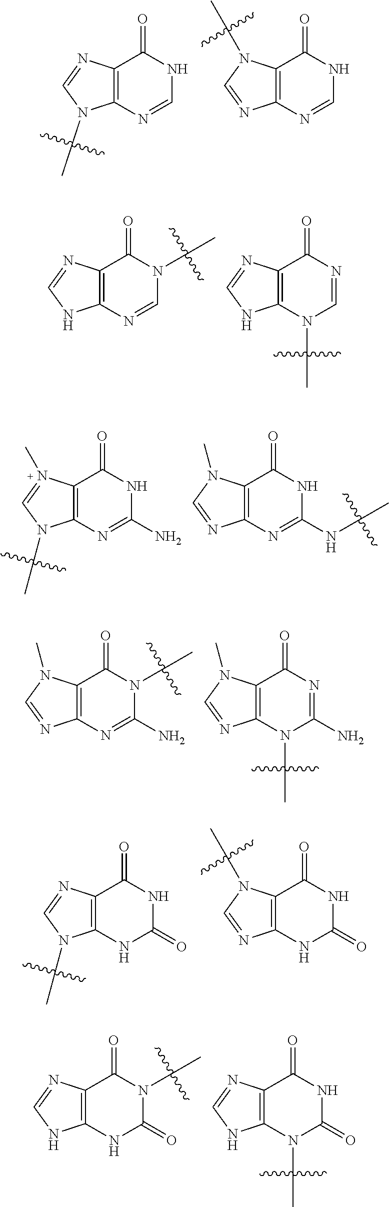

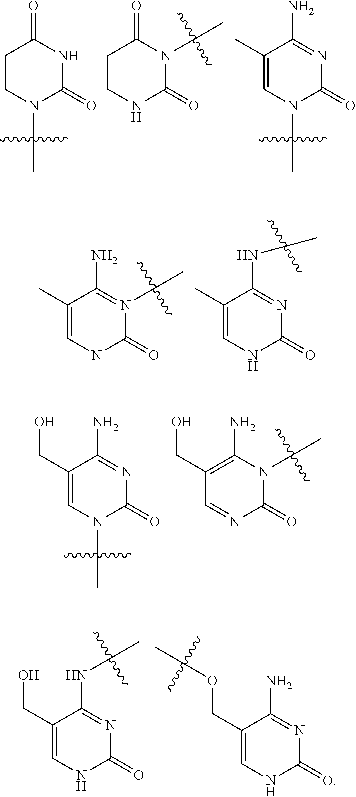

11: The method of claim 1, where the disease or disorder is responsive to regulation of TOR function, and wherein the composition comprises one or more MOGLs selected from the group consisting of: 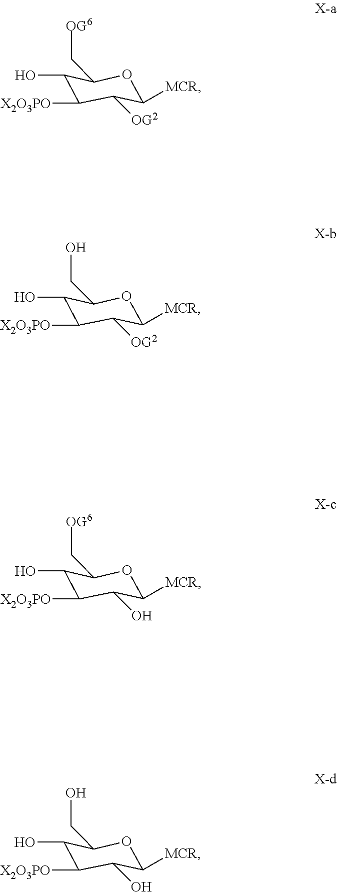
where -MCR—comprises a C₃₋₁₂ alpha beta unsaturated acyl group.

12: The method of claim 11, wherein -MCR comprises a C₃₋₈ alpha beta unsaturated acyl group, or wherein the moiety -MCR comprises a C₄₋₈ alpha beta unsaturated acyl group, or wherein the moiety -MCR comprises an acyl group corresponding to an ester of acrylic acid, methylacrylic acid, crotonic acid, methyl crotonic acid, valeric acid, 3-methylcrotonic acid, or tiglic acid.

13: The method of claim 11, wherein -MCR is selected from the group consisting of: crotonate, tiglate, valerate, acrylate, methacrylate, cinnamate, 2-imidazoleacrylate and urocanate.

14: A method for treating a disease or disorder responsive to regulation of proteasome function, comprising administering to a patient in need thereof a therapeutically effective amount of a composition comprising one or more MOGLs of Formula A-1: 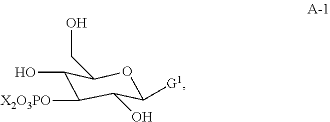
or a pharmaceutically acceptable salt thereof,
G¹ is an optionally substituted moiety selected from the group consisting of: N-linked heteroaryl, —OR¹⁰ and —OC(O)R¹¹;
X is, independently at each occurrence, selected from the group consisting of —H, an optionally substituted phosphate or polyphosphate moiety, M⁺, and Z⁺;
R¹⁰ and R¹¹ are each independently selected from the group consisting of: optionally substituted C₁₋₃₂ aliphatic, optionally substituted C₁₋₃₂ heteroaliphatic, optionally substituted aryl, and optionally substituted heteroaryl;
M⁺ is any metal cation; and
Z⁺ is an organic or inorganic ‘onium’ group comprising at least one nitrogen-, phosphorous-, or sulfur-based cation.

15: A method comprising administering to a mammal a composition comprising a therapeutically effective amount of one or more MOGLs of Formula I: 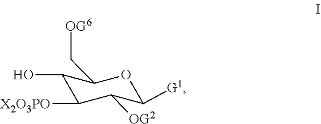
or a pharmaceutically acceptable salt thereof
wherein:
G¹ is an optionally substituted moiety selected from the group consisting of: N-linked heterocycle (e.g., N-linked heteroaryl), —OR¹⁰ and —OC(O)R¹¹;
G² is an optionally substituted aliphatic, aromatic (e.g., aryl), heteroaromatic, or heteroaliphatic acyl group;
X is, independently at each occurrence, selected from the group consisting of —H, an optionally substituted phosphate or polyphosphate moiety, M⁺, and Z⁺;
G⁶ is an optionally substituted aliphatic, aromatic (e.g., aryl), heteroaromatic, or heteroaliphatic acyl group; and
wherein, R¹⁰ and R¹¹ are each independently selected from the group consisting of: optionally substituted C₁₋₃₂ aliphatic, optionally substituted C₁₋₃₂ heteroaliphatic, optionally substituted aryl, and optionally substituted heteroaryl.

16: The method of claim 15, where the disease or disorder is cancer or another other kinase dependent disorder or disease such as hypertension, Parkinson's disease, and autoimmune disease, or a disorder that results in or arises from changes to nucleotide synthesis including cancer and viral diseases,
wherein the one or more MOGLs is selected from the group consisting of: 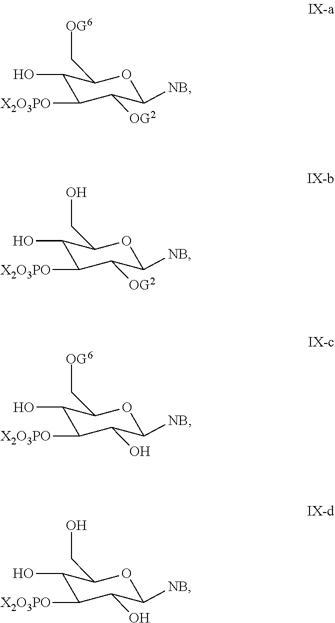
where —NB comprises an aromatic moiety, a nucleobase, or a derivative or precursor of a nucleobase linked to the glucose through any suitable atom.

17: The method of claim 15, where the disease or disorder is a neurological disease, and wherein the one or more MOGLs selected from the group consisting of: 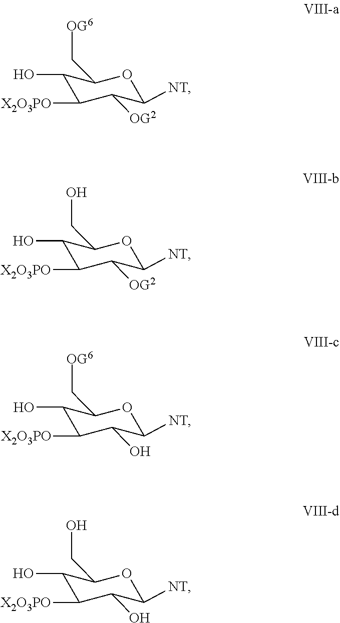
where the moiety —NT comprises a neurotransmitter, or a derivative or precursor of a neurotransmitter linked to the glucose through any suitable atom.

18: The method of claim 15, where the disease or disorder is one responsive to regulation of TOR function, and wherein the composition comprises one or more MOGLs selected from the group consisting of: 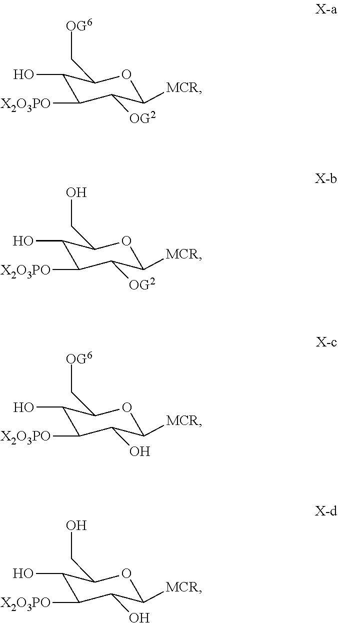
where -MCR comprises a C₃₋₁₂ alpha beta unsaturated acyl group.

19: A method comprising administering to a mammal a composition comprising a therapeutically effective amount of one or more MOGLs of Formula A-1 or A-2: 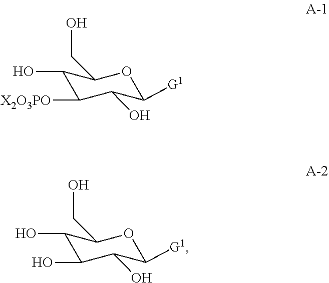
or a pharmaceutically acceptable salt thereof,
G¹ is an optionally substituted moiety selected from the group consisting of: N-linked heteroaryl, —OR¹ and —OC(O)R¹¹;
X is, independently at each occurrence, selected from the group consisting of —H, an optionally substituted phosphate or polyphosphate moiety, M⁺, and Z⁺;
R¹⁰ and R¹¹ are each independently selected from the group consisting of: optionally substituted C₁₋₃₂ aliphatic, optionally substituted C₁₋₃₂ heteroaliphatic, optionally substituted aryl, and optionally substituted heteroaryl;
M⁺ is any metal cation; and
Z⁺ is an organic or inorganic ‘onium’ group comprising at least one nitrogen-, phosphorous-, or sulfur-based cation.

20: The method of claim 1, wherein the composition is a pharmaceutical composition comprising a pharmaceutically acceptable excipient or carrier.

21: A compound of Formula I, Formula II, Formula III, Formula IV, Formula V, Formula VI, Formula VII, Formula XI-a, Formula XI-b, Formula XI-c, Formula XI-d, Formula XI-e, Formula XI-f, Formula XI-g, Formula A-1, Formula A-2, or Table S5.

22: The compound of claim 21, wherein said compound is a compound of Formula II: 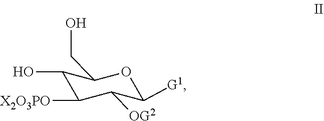
or a pharmaceutically acceptable salt thereof,
wherein:
G¹ is an optionally substituted moiety selected from the group consisting of: N-linked heterocycle, —OR¹⁰ and —OC(O)R¹¹;
G² is an optionally substituted aliphatic, aromatic, heteroaromatic, or heteroaliphatic acyl group;
R¹⁰ and R¹¹ are each independently selected from the group consisting of: optionally substituted C₁₋₃₂ aliphatic, optionally substituted C₁₋₃₂ heteroaliphatic, optionally substituted aryl, and optionally substituted heteroaryl;
X is, independently at each occurrence, selected from the group consisting of —H, an optionally substituted phosphate or polyphosphate moiety, M⁺, and Z⁺;
M⁺ is any metal cation; and
Z⁺ is an organic or inorganic ‘onium’ group comprising at least one nitrogen-, phosphorous-, or sulfur-based cation.

23: The compound of claim 21,
wherein said compound is a compound of Formula III: 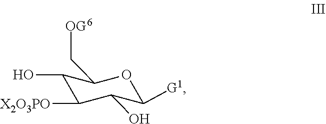
or a pharmaceutically acceptable salt thereof,
wherein:
G¹ is an optionally substituted moiety selected from the group consisting of: N-linked heterocycle, —OR¹⁰ and —OC(O)R¹¹;
X is, independently at each occurrence, selected from the group consisting of —H, an optionally substituted phosphate or polyphosphate moiety, M⁺, and Z⁺;
G⁶ is an optionally substituted aliphatic, aromatic, heteroaromatic, or heteroaliphatic acyl group;
where, R¹⁰ and R¹¹ are each independently selected from the group consisting of: optionally substituted C₁₋₃₂ aliphatic, optionally substituted C₁₋₃₂ heteroaliphatic, optionally substituted aryl, and optionally substituted heteroaryl;
M⁺ is any metal cation; and
Z⁺ is an organic or inorganic ‘onium’ group comprising at least one nitrogen-, phosphorous-, or sulfur-based cation.

24: The compound of claim 21, wherein said compound is a compound of Formula IV: 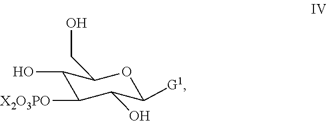
or a pharmaceutically acceptable salt thereof,
wherein:
G¹ is an optionally substituted moiety selected from the group consisting of: N-linked heterocycle, —OR¹⁰ and —OC(O)R¹¹;
X is, independently at each occurrence, selected from the group consisting of —H, an optionally substituted phosphate or polyphosphate moiety, M⁺, and Z⁺;
R¹⁰ and R¹¹ are each independently selected from the group consisting of: optionally substituted C₁₋₃₂ aliphatic, optionally substituted C₁₋₃₂ heteroaliphatic, optionally substituted aryl, and optionally substituted heteroaryl;
M⁺ is any metal cation; and
Z⁺ is an organic or inorganic ‘onium’ group comprising at least one nitrogen-, phosphorous-, or sulfur-based cation.

25: The compound of claim 21, wherein said compound is a compound of Formula V: 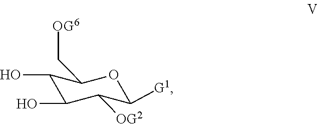
or a pharmaceutically acceptable salt thereof,
G¹ is an optionally substituted moiety selected from the group consisting of: N-linked heterocycle, —OR¹⁰ and —OC(O)R¹¹;
G² is an optionally substituted aliphatic, aromatic, heteroaromatic, or heteroaliphatic acyl group;
G⁶ is an optionally substituted aliphatic, aromatic, heteroaromatic, or heteroaliphatic acyl group; and
R¹⁰ and R¹¹ are each independently selected from the group consisting of: optionally substituted C₁₋₃₂ aliphatic, optionally substituted C₁₋₃₂ heteroaliphatic, optionally substituted aryl, and optionally substituted heteroaryl.

26: The compound of claim 21, wherein said compound is a compound of Formula VI: 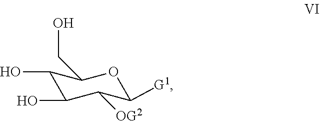
or a pharmaceutically acceptable salt thereof,
G¹ is an optionally substituted moiety selected from the group consisting of: N-linked heterocycle, —OR¹⁰ and —OC(O)R¹¹;
G² is an optionally substituted aliphatic, aromatic, heteroaromatic, or heteroaliphatic acyl group; and
R¹⁰ and R¹¹ are each independently selected from the group consisting of: optionally substituted C₁₋₃₂ aliphatic, optionally substituted C₁₋₃₂ heteroaliphatic, optionally substituted aryl, and optionally substituted heteroaryl.

27: The compound of claim 21, wherein said compound is a compound of Formula VII: 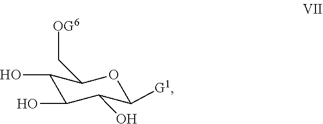
or a pharmaceutically acceptable salt thereof,
G¹ is an optionally substituted moiety selected from the group consisting of: N-linked heterocycle, —OR¹⁰ and —OC(O)R¹¹;
G⁶ is an optionally substituted aliphatic, aromatic, heteroaromatic, or heteroaliphatic acyl group; and
R¹⁰ and R¹¹ are each independently selected from the group consisting of: optionally substituted C₁₋₃₂ aliphatic, optionally substituted C₁₋₃₂ heteroaliphatic, optionally substituted aryl, and optionally substituted heteroaryl.

28: The compound of claim 21, wherein said compound is a compound of Formulae XI-a, XI-b, XI-c, XI-d, XI-e, XI-f, or XI-g: 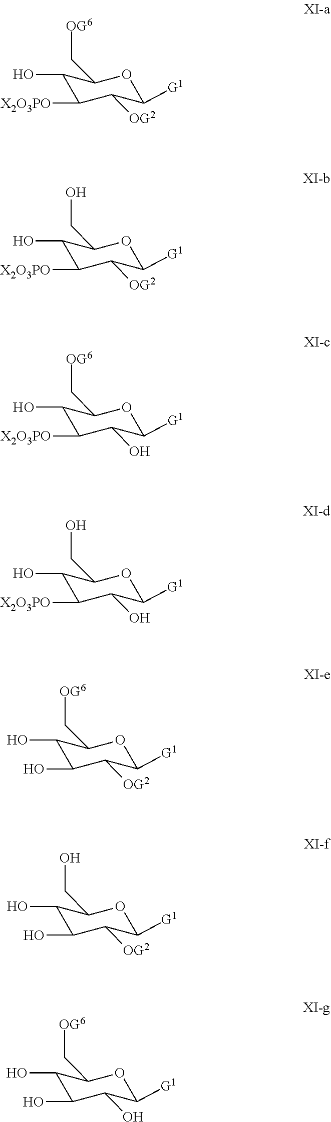
or a pharmaceutically acceptable salt thereof, wherein:
G¹ is —NRⁿ¹Rⁿ², wherein Rⁿ¹ and Rⁿ² are each independently selected from the group consisting of: hydrogen, optionally substituted C₁₋₂₀ aliphatic, optionally substituted C₁₋₂₀ acyl, optionally substituted aryl, and optionally substituted heterocyclic;
G² is an optionally substituted aliphatic, aromatic, heteroaromatic, or heteroaliphatic acyl group;
X is, independently at each occurrence, selected from the group consisting of —H, an optionally substituted phosphate or polyphosphate moiety, M⁺, and Z⁺;
G⁶ is an optionally substituted aliphatic, aromatic, heteroaromatic, or heteroaliphatic acyl group,
R¹⁰ and R¹¹ are each independently selected from the group consisting of: optionally substituted C₁₋₃₂ aliphatic, optionally substituted C₁₋₃₂ heteroaliphatic, optionally substituted aryl, and optionally substituted heteroaryl;
M⁺ is any metal cation; and
Z⁺ is an organic or inorganic ‘onium’ group comprising at least one nitrogen-, phosphorous-, or sulfur-based cation.

29: The compound of claim 21, wherein said compound is a compound of Formula A-1 or A-2: 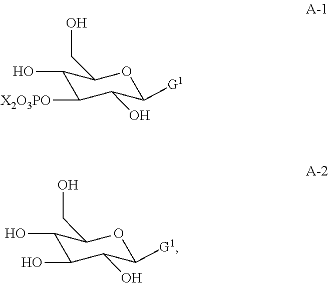
or a pharmaceutically acceptable salt thereof,
G¹ is an optionally substituted moiety selected from the group consisting of: N-linked heteroaryl, —OR¹⁰ and —OC(O)R¹¹;
X is, independently at each occurrence, selected from the group consisting of —H, an optionally substituted phosphate or polyphosphate moiety, M⁺, and Z⁺;
R¹⁰ and R¹¹ are each independently selected from the group consisting of: optionally substituted C₁₋₃₂ aliphatic, optionally substituted C₁₋₃₂ heteroaliphatic, optionally substituted aryl, and optionally substituted heteroaryl;
M⁺ is any metal cation; and
Z⁺ is an organic or inorganic ‘onium’ group comprising at least one nitrogen-, phosphorous-, or sulfur-based cation.

30: The compound of claim 21, wherein said compound is a compound of Table S5, or a pharmaceutically acceptable salt thereof.

31: A pharmaceutical composition comprising a compound of claim 21 and a pharmaceutically acceptable carrier or excipient.

32: The compound of claim 21, wherein said compound is a compound of Formula I: 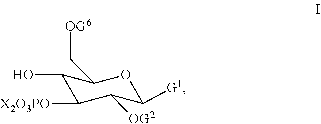
or a pharmaceutically acceptable salt thereof,
wherein:
G¹ is an optionally substituted moiety selected from the group consisting of: N-linked heterocycle, —OR¹⁰ and —OC(O)R¹¹;
G² is an optionally substituted aliphatic, aromatic, heteroaromatic, or heteroaliphatic acyl group;
X is, independently at each occurrence, selected from the group consisting of —H, an optionally substituted phosphate or polyphosphate moiety, M⁺, and Z⁺;
G⁶ is an optionally substituted aliphatic, aromatic, heteroaromatic, or heteroaliphatic acyl group,
R¹⁰ and R¹¹ are each independently selected from the group consisting of: optionally substituted C₁₋₃₂ aliphatic, optionally substituted C₁₋₃₂ heteroaliphatic, optionally substituted aryl, and optionally substituted heteroaryl;
M⁺ is any metal cation; and
Z⁺ is an organic or inorganic ‘onium’ group comprising at least one nitrogen-, phosphorous-, or sulfur-based cation.


SEQUENCE LISTING (4 sequences)

SEQ ID NO: 1 - protein, 673 aa, Caenorhabditis elegans
MLKPATVLLLIQMYVCKRVQLSSGTIEGKILNISYSPLGNQSATVFLGIPFVEPPIGDLRYRKPRPPKSWEGVLVTNEYKSACMSNATKTYKNKFGGPISEDCLYLNVLTNEYCLENKNCSVMMIVHGGGYLTESASTFNPEILINNFVGQGRNIVVVTFNYRLGLFSFGQFNGDRGDKNFGLYDMIESVNWVRREIENFGGNKNRITLAGHSAGASMIVAFTSSPLTKGLVHQQIIMSAPMTNMSKKSNFKGMTVMAQMVGCLSEEIGFNKLSEEQVENTYSCLRKKSAQQILDAQLWLLQNSTYFLGAPPIDEHFLTDYPENLYASKSIYPINTLIGTTTLEVEESSYIIDPAFADKKVELLENLCDHIGYVLYEEPETFSKKCQKQYMNGNSSMNLSNEMEFYTPAIDFADSHTSGNTKVFLYSYDYRGAGPAYDRYLEVRSPHHSEDLIYVFGTHRGIFAPKDYIIEKIYSGMFADFVNFENPLPSGDQKWNQYTKENREHFLINFDKNFITPGMRDNYYTEAYEFWSTVGKKSFKEEWSPSLDTFTCALVISPLVSHMKQTTTAFDKTFEQTELLYKEEVNFLKREKLERTQELKMETKRRDKALRIQNRKNGLANKEITEGDEEDESSKLDILLIISAGTLFGGILYVTLPNVILQKRARDGYELLS

SEQ ID NO: 2 - protein, 676 aa, Caenorhabditis elegans
MIFKFVVLLWFSAQFAISTKVQLSTGIIEGKQLNSTYSPFGNQSAIAFLGIPYVEPPIGDLRFRKPRPQKTWSGVLETKKFKPACMSNSTKTYKNGGIGGPISEDCLYLNVLTNQYCLENKNCSVVIIVHGGGYLFESAATFNHEILVNNFVGQDRNIVVVTINYRLGIFGFGHLNGFEGDKNVGIFDTLEAVKWVRREIKQFGGNKDRITLAGHSAGAGVVAAFTISPLTKGLIHQQIVMSGPMTNMSKKSNFKGMTAMAERVGCHSKKYGFNKLPEHEVEQVYSCLRKKPAQELLDAQLWLLQNSTFYFGAPPVDGEFITDYPDNLYSEKSIFSINTLLGTTTRELQDTGYISNSNYSKFETKETFLKNLCDQIGIEIYKESEDFSSKCQKWYINGSDSKSLSDDMEFYTQAINLADAHARKGKKVYMYSYAYSGAGPAFKKYMKVKSPHHSEDLIYTFGTHRGVFAPKDYVIEYIYSGMFADFINFEDPSPSTDQPWLQYTKENKEYFLVDFDQNLTMPGMKKNYYAKAYEFWSTAGQKSFKEEWSPSLDTFIITNLVAPIITHMDQITFDVDKTVEQMEILKIERENFLKSEKLKRKSELEMSRWIKKGRKRVINKRMGFMPVEIIENEESESDGGFSIGLLLFGGTLLGGIVYVSSYQFFRRSRNGYQLIN

SEQ ID NO: 3 - protein, 665 aa, Caenorhabditis briggsae
MFFQTFLVVVIANTALSQRILLSTGEIEGKTLNATYSPLGNQSAIVFLGIPYVAPPLGNLRFHKPKPPIPWKGILETKAYRPACMSNATKTYKNSVGGPISEDCLYANVFTNEYCLEKKNCSVMIAIHGGGFLFESASAFNPEILINNFVGQNRNIVVVSLNYRLGAFGFGHLAGDVGERNLGLFDILAGVKWVRQEIHSFGGNKHRITLMGHSAGAGLVALFSISPLTKGLIHQQIVMSGPLTNLSKKSNYKAMTKVTQIVGCLPEDVGFEPLSKVQLKKAYSCLRSKSAQEILDAEMFVMRDTTYYFGAPHVDGEFIIDYPDHIFQDKSVFPINTMLGTTTAELRDSIYINDPKNADKKEELLQNMCEHIGFEIYKEPERFVDQCVKNYGNATQAQYLSDDMEFYSRAISTANLHLSENTKVYLYSYAYSGAGRAFNKYLNVPSPHHSEDLIYVFGTHRGVFAPKDYIIEQIYTGMFANFINFGNPTLSEDQPWLQYTKEKREHFVINFDANFTMPGMRELYYTNAMNFWHNAGEKTFREHFSKSLDTFHIVNLNTPIVSHIDGISTDVDKTFEQTEILFSEREQFLAELKLKRNIGKVAGNGWGREAAARIVQREAELEENGGGINILLIIFAGTLVFGILYVTISHFCLHHRSREGYQLLK

SEQ ID NO: 4 - DNA, 20 bp, synthetic construct
  source 1..20: mol_type = other DNA
cattactcatacaagctgga